SEQUENCE LISTING (301 sequences)

SEQ ID NO: 1 - protein, 37 aa, synthetic construct
  REGION 1..37: note = ECD1 of US28, HCMV strain DB
MTPTTTTAELTTEFDYDEAATPCVFTDVLNQSKPVTL

SEQ ID NO: 2 - protein, 11 aa, synthetic construct
  REGION 1..11: note = ECD2 of US28, HCMV strain DB
QYLLDHNSLAS

SEQ ID NO: 3 - protein, 17 aa, synthetic construct
  REGION 1..17: note = ECD3 of US28, HCMV strain DB
TKKNNQCMTDYDYLEVS

SEQ ID NO: 4 - protein, 24 aa, synthetic construct
  REGION 1..24: note = ECD4 of US28, HCMV strain DB
VDTLKLLKWISSSCEFERSLKRAL

SEQ ID NO: 5 - protein, 354 aa, synthetic construct
  REGION 1..354: note = US28, HCMV strain DB
MTPTTTTAELTTEFDYDEAATPCVFTDVLNQSKPVTLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKNNQCMTDYDYLEVSYPIILNVELMLGAFVIPLSVISYCYYRISRIVAVSQSRHKGRIVRVLIAVVLVFIIFWLPYHLTLFVDTLKLLKWISSSCEFERSLKRALILTESLAFCHCCLNPLLYVFVGTKFRQELHCLLAEFRQRLFSRDVSWYHSMSFSRRSSPSRRETSSDTLSDEVCRVSQIIP

SEQ ID NO: 6 - protein, 17 aa, synthetic construct
  REGION 1..17: note = ECD3 4N variant group of HCMV strains
TKKNNQCMTDYDYLEVS

SEQ ID NO: 7 - protein, 17 aa, synthetic construct
  REGION 1..17: note = ECD3 4D variant group of HCMV strains
TKKDNQCMTDYDYLEVS

SEQ ID NO: 8 - protein, 8 aa, synthetic construct
  REGION 1..8: note = VH-CDR1 sequence of 1D3
GFTFTDYY

SEQ ID NO: 9 - protein, 10 aa, synthetic construct
  REGION 1..10: note = VH-CDR2 sequence of 1D3
IRSKANGYTT

SEQ ID NO: 10 - protein, 12 aa, synthetic construct
  REGION 1..12: note = VH-CDR3 sequence of 1D3
ARDERRTAWLAY

SEQ ID NO: 11 - protein, 140 aa, synthetic construct
  REGION 1..140: note = Immature form of the recombinantly expressed VH sequence of 1D3, wherein positions 1-19 correspond to a leader sequence
MKLWLNWIFLVTLLNGIQCEVKLVESGGGLVQPGGSLRLACATSGFTFTDYYMSWVRQPPGKALEWLGFIRSKANGYTTEYSASVKGRFTISRDNSQSILYLQMNTLRSEDSATYYCARDERRTAWLAYWGQGTLVTVSA

SEQ ID NO: 12 - protein, 121 aa, synthetic construct
  REGION 1..121: note = Mature form of VH sequence of 1D3, without the leader sequence
EVKLVESGGGLVQPGGSLRLACATSGFTFTDYYMSWVRQPPGKALEWLGFIRSKANGYTTEYSASVKGRFTISRDNSQSILYLQMNTLRSEDSATYYCARDERRTAWLAYWGQGTLVTVSA

SEQ ID NO: 13 - protein, 19 aa, synthetic construct
  REGION 1..19: note = Leader sequence of recombinantly expressed VH sequence of 1D3
MKLWLNWIFLVTLLNGIQC

SEQ ID NO: 14 - protein, 11 aa, synthetic construct
  REGION 1..11: note = VL-CDR1 sequence of 1D3
QSIVHSNGNTY

SEQ ID NO: 15 - skipped

SEQ ID NO: 16 - protein, 10 aa, synthetic construct
  REGION 1..10: note = VL-CDR3 sequence of 1D3
FQGSHVPTWT

SEQ ID NO: 17 - protein, 132 aa, synthetic construct
  REGION 1..132: note = Immature form of the recombinantly expressed VL sequence of 1D3, wherein positions 1-19 correspond to a leader sequence
MKLPVRLLVLMVWIPASSSDVLMTQTPLSLPVSLGDQASISCRSSQSIVHSNGNTYLDWYLQKPGQSPKLLIYKVSNRFSGVPDRFSGSGSGTDFTLKISRVEAEDLGVYYCFQGSHVPTWTFGGGTKLEIK

SEQ ID NO: 18 - protein, 113 aa, synthetic construct
  REGION 1..113: note = Mature form of VL sequence of 1D3, without the leader sequence
DVLMTQTPLSLPVSLGDQASISCRSSQSIVHSNGNTYLDWYLQKPGQSPKLLIYKVSNRFSGVPDRFSGSGSGTDFTLKISRVEAEDLGVYYCFQGSHVPTWTFGGGTKLEIK

SEQ ID NO: 19 - protein, 19 aa, synthetic construct
  REGION 1..19: note = Leader sequence of recombinantly expressed VL sequence of 1D3
MKLPVRLLVLMVWIPASSS

SEQ ID NO: 20 - protein, 464 aa, synthetic construct
  REGION 1..464: note = Complete sequence of the heavy chain of 1D3
MKLWLNWIFLVTLLNGIQCEVKLVESGGGLVQPGGSLRLACATSGFTFTDYYMSWVRQPPGKALEWLGFIRSKANGYTTEYSASVKGRFTISRDNSQSILYLQMNTLRSEDSATYYCARDERRTAWLAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 21 - protein, 239 aa, synthetic construct
  REGION 1..239: note = Complete sequence of the light chain of 1D3
MKLPVRLLVLMVWIPASSSDVLMTQTPLSLPVSLGDQASISCRSSQSIVHSNGNTYLDWYLQKPGQSPKLLIYKVSNRFSGVPDRFSGSGSGTDFTLKISRVEAEDLGVYYCFQGSHVPTWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 22 - DNA, 24 bp, synthetic construct
  misc_feature 1..24: note = VH-CDR1 sequence of 1D3
  source 1..24: mol_type = other DNA
gggttcaccttcactgattactat

SEQ ID NO: 23 - DNA, 30 bp, synthetic construct
  misc_feature 1..30: note = VH-CDR2 sequence of 1D3
  source 1..30: mol_type = other DNA
attagaagcaaagctaatggttacacaaca

SEQ ID NO: 24 - DNA, 36 bp, synthetic construct
  misc_feature 1..36: note = VH-CDR3 sequence of 1D3
  source 1..36: mol_type = other DNA
gcaagagatgagcgccgtactgcctggcttgcttac

SEQ ID NO: 25 - DNA, 422 bp, synthetic construct
  misc_feature 1..422: note = Immature form of the recombinantly expressed VH sequence of 1D3
  source 1..422: mol_type = other DNA
atgaagttgtggctgaactggattttccttgtaacacttttaaatggtatccagtgtgaggtgaagctggtggagtctggaggaggcttggtacagccggggggttctctgagactcgcctgtgcaacttctgggttcaccttcactgattactatatgagctgggtccgccagcctccaggaaaggcacttgagtggctgggttttattagaagcaaagctaatggttacacaacagagtacagtgcatctgttaagggtcggttcaccatctccagagataattcccaaagcatcctctatcttcaaatgaacaccctgagatctgaggacagtgccacttattactgtgcaagagatgagcgccgtactgcctggcttgcttactggggccaagggactctggtcactgtctctgcagc

SEQ ID NO: 26 - DNA, 365 bp, synthetic construct
  misc_feature 1..365: note = Mature form of VH sequence of 1D3
  source 1..365: mol_type = other DNA
gaggtgaagctggtggagtctggaggaggcttggtacagccggggggttctctgagactcgcctgtgcaacttctgggttcaccttcactgattactatatgagctgggtccgccagcctccaggaaaggcacttgagtggctgggttttattagaagcaaagctaatggttacacaacagagtacagtgcatctgttaagggtcggttcaccatctccagagataattcccaaagcatcctctatcttcaaatgaacaccctgagatctgaggacagtgccacttattactgtgcaagagatgagcgccgtactgcctggcttgcttactggggccaagggactctggtcactgtctctgcagc

SEQ ID NO: 27 - DNA, 57 bp, synthetic construct
  misc_feature 1..57: note = Leader sequence of recombinantly expressed VH sequence of 1D3
  source 1..57: mol_type = other DNA
atgaagttgtggctgaactggattttccttgtaacacttttaaatggtatccagtgt

SEQ ID NO: 28 - DNA, 33 bp, synthetic construct
  misc_feature 1..33: note = VL-CDR1 sequence of 1D3
  source 1..33: mol_type = other DNA
cagagcattgtacatagtaatggaaacacctat

SEQ ID NO: 29 - skipped

SEQ ID NO: 30 - DNA, 30 bp, synthetic construct
  misc_feature 1..30: note = VL-CDR3 sequence of 1D3
  source 1..30: mol_type = other DNA
tttcaaggttcacatgttcccacgtggacg

SEQ ID NO: 31 - DNA, 396 bp, synthetic construct
  misc_feature 1..396: note = Immature form of the recombinantly expressed VL sequence of 1D3
  source 1..396: mol_type = other DNA
atgaagttgcctgttaggctgttggtgctgatggtctggattcctgcttccagcagtgatgttttgatgacccaaactccactctccctgcctgtcagtcttggagatcaagcctccatctcttgcagatctagtcagagcattgtacatagtaatggaaacacctatttagactggtaccttcagaaaccaggccagtctccaaagctcctgatctacaaagtttccaaccgattttctggggtcccagacaggttcagtggcagtggatcagggacagatttcacactcaaaatcagcagagtggaggctgaggatctgggagtttattactgctttcaaggttcacatgttcccacgtggacgttcggtggaggcaccaagctggaaatcaaa

SEQ ID NO: 32 - DNA, 339 bp, synthetic construct
  misc_feature 1..339: note = Mature form of VL sequence of 1D3
  source 1..339: mol_type = other DNA
gatgttttgatgacccaaactccactctccctgcctgtcagtcttggagatcaagcctccatctcttgcagatctagtcagagcattgtacatagtaatggaaacacctatttagactggtaccttcagaaaccaggccagtctccaaagctcctgatctacaaagtttccaaccgattttctggggtcccagacaggttcagtggcagtggatcagggacagatttcacactcaaaatcagcagagtggaggctgaggatctgggagtttattactgctttcaaggttcacatgttcccacgtggacgttcggtggaggcaccaagctggaaatcaaa

SEQ ID NO: 33 - DNA, 57 bp, synthetic construct
  misc_feature 1..57: note = Leader sequence of recombinantly expressed VL sequence of 1D3
  source 1..57: mol_type = other DNA
atgaagttgcctgttaggctgttggtgctgatggtctggattcctgcttccagcagt

SEQ ID NO: 34 - DNA, 1395 bp, synthetic construct
  misc_feature 1..1395: note = Complete sequence of the heavy chain of 1D3
  source 1..1395: mol_type = other DNA
atgaagttgtggctgaactggattttccttgtaacacttttaaatggtatccagtgtgaggtgaagctggtggagtctggaggaggcttggtacagccggggggttctctgagactcgcctgtgcaacttctgggttcaccttcactgattactatatgagctgggtccgccagcctccaggaaaggcacttgagtggctgggttttattagaagcaaagctaatggttacacaacagagtacagtgcatctgttaagggtcggttcaccatctccagagataattcccaaagcatcctctatcttcaaatgaacaccctgagatctgaggacagtgccacttattactgtgcaagagatgagcgccgtactgcctggcttgcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 35 - DNA, 720 bp, synthetic construct
  misc_feature 1..720: note = Complete sequence of the light chain of 1D3
  source 1..720: mol_type = other DNA
atgaagttgcctgttaggctgttggtgctgatggtctggattcctgcttccagcagtgatgttttgatgacccaaactccactctccctgcctgtcagtcttggagatcaagcctccatctcttgcagatctagtcagagcattgtacatagtaatggaaacacctatttagactggtaccttcagaaaccaggccagtctccaaagctcctgatctacaaagtttccaaccgattttctggggtcccagacaggttcagtggcagtggatcagggacagatttcacactcaaaatcagcagagtggaggctgaggatctgggagtttattactgctttcaaggttcacatgttcccacgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 36 - protein, 354 aa, synthetic construct; identical to SEQ ID NO: 5
  REGION 1..354: note = US28 Toledo strain

SEQ ID NO: 37 - protein, 354 aa, synthetic construct
  REGION 1..354: note = US28 Towne strain
MTPTTTTAELTTEFDYDEDATPCVFTDVLNQSKPVTLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNVELMLGAFVIPLSVISYCYYRISRIVAVSQSRHKGRIVRVLIAVVLVFIIFWLPYHLTLFVDTLKLLKWISSSCEFERSLKRALILTESLAFCHCCLNPLLYVFVGTKFRQELHCLLAEFRQRLFSRDVSWYHSMSFSRRSSPSRRETSSDTLSDEVCRVSQIIP

SEQ ID NO: 38 - protein, 354 aa, synthetic construct
  REGION 1..354: note = US28 VR 1814 strain
MTPTTTTAELTTEFDYDEDATPCVFTDVLNQSKPVTLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNVELMLGAFVIPLSVISYCYYRISRIVAVSQSRHKGRIVRVLIAVVLVFIIFWLPYHLTLFVDTLKLLKWISSSCEFERSLKRALILTESLAFCHCCLNPLLYVFVGTKFRQELHCLLAEFRQRLFSRDVSWYHSMSFSRRGSPSRRETSSDTLSDEVCRVSQIIP

SEQ ID NO: 39 - protein, 354 aa, synthetic construct
  REGION 1..354: note = US28 TB40/E strain
MTPTTTTTELTTEFEYDLGATPCTFTDVLNQSKPVTLFLYGVVFLFGSVGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNVELMLGAFVIPLSVISYCYYRISRIVAVSQSRHKGRIVRVLIAVVLVFIIFWLPYHLTLFVDTLKLLKWISSSCEFERSLKRALILTESLAFCHCCLNPLLYVFVGTKFRQELHCLLAEFRQRLFSRDVSWYHSMSFSRRSSPSRRETSSDTLSDEVCRVSQIIP

SEQ ID NO: 40 - protein, 354 aa, synthetic construct; identical to SEQ ID NO: 38
  REGION 1..354: note = US28 Merlin strain

SEQ ID NO: 41 - protein, 354 aa, synthetic construct
  REGION 1..354: note = US28 JP strain
MTPTTTTAELTTEFDYDEAATPCVFTDVLNQSKPVTLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNVELMLGAFVIPLSVISYCYYRISRIVAVSQSRHKGRIVRVLIAVVLVFIIFWLPYHLTLFVDTLKLLKWISSSCEFERSLKRALILTESLAFCHCCLNPLLYVFVGTKFRQELHCLLAEFRQRLFSRDVSWYHSMSFSRRSSPSRRETSSDTLSDEVCRVSQIIP

SEQ ID NO: 42 - protein, 354 aa, synthetic construct; identical to SEQ ID NO: 37
  REGION 1..354: note = US28 Ad169 strain

SEQ ID NO: 43 - protein, 354 aa, synthetic construct
  REGION 1..354: note = US28 AF1 strain
MTPPTTTAELTTEFDYDEAATPCVFTDVLNQSKPVTLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNVELMLGAFVIPLSVISYCYYRISRIVAVSQSRHKGRIVRVLIAVVLVFIIFWLPYHLTLFVDTLKLLKWISSSCEFERSLKRALILTESLAFCHCCLNPLLYVFVGTKFRQELHCLLAEFRQRLFSRDVSWYHSMSFSRRSSPSRRETSSDTLSDEVCRVSQIIP

SEQ ID NO: 44 - protein, 354 aa, synthetic construct
  REGION 1..354: note = US28 VHL/E strain
MTPTTTTAELTTEFDYDDEATPCVLTDVLNQSKPVTLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNVELMLGAFVIPLSVISYCYYRISRIVAVSQSRHKGRIVRVLIAVVLVFIIFWLPYHLTLFVDTLKLLKWISSSCEFEKSLKRALILTESLAFCHCCLNPLLYVFVGTKFRQELHCLLAEFRQRLFSRDVSWYHSMSFSRRSSPSRRETSSDTLSDEACRVSQIIP

SEQ ID NO: 45 - protein, 354 aa, synthetic construct
  REGION 1..354: note = US28 DAVIS strain
MTPTTTTAELTTEFDYDEDATPCVFTDVLNQSKPVTLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNIELMLGAFVIPLSVISYCYYRISRIVAVSQSRHKGRIVRVLIAVVLVFIIFWLPYHLTLFVDTLKLLKWISSSCEFEKSLKRALILTESLAFCHCCLNPLLYVFVGTKFRQELHCLLAEFRQRLFSRDVSWYHSMSFSRRSSPSRRETSSDTLSDEACRVSQIIP

SEQ ID NO: 46 - protein, 354 aa, synthetic construct
  REGION 1..354: note = US28 TR strain
MTPTTTTAELTTEFDYDEAATPCVFTDVLNQSKPVTLFLYGVVFIFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKNNQCMTDYDYLEVSYPIILNVELMLGAFVIPLSVISYCYYRISRIVAVSQSRHKGRIVRVLIAVVLVFIIFWLPYHLTLFVDTLKLLKWISSSCEFEKSLKRALILTESLAFCHCCLNPLLYVFVGTKFRQELHCLLAEFRQRLFSRDVSWYHSMSFSRRSSPSRRETSSDTLSDEVCRVSQIIP

SEQ ID NO: 47 - protein, 367 aa, synthetic construct
  REGION 1..367: note = US28 fusion protein, comprising US28 sequence from HCMV DB strain
MTPTTTTAELTTEFDYDEAATPCVFTDVLNQSKPVTLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKNNQCMTDYDYLEVSYPIILNVELMLGAFVIPLSVISYCYYRISRIVAVSQSRHKGRIVRVLIAVVLVFIIFWLPYHLTLFVDTLKLLKWISSSCEFERSLKRALILTESLAFCHCCLNPLLYVFVGTKFRQELHCLLAEFRQRLFSRDVSWYHSMSFSRRSSPSRRETSSDTLSDEVCRVSQIIPAAALEGSHHHHHH

SEQ ID NO: 48 - protein, 119 aa, synthetic construct [sequence omitted]

SEQ ID NO: 49 - protein, 5 aa, synthetic construct
  REGION 1..5: note = OKT3 VH-CDR1 sequence
RYTMH

SEQ ID NO: 50 - protein, 16 aa, synthetic construct
  REGION 1..16: note = OKT3 VH-CDR2 sequence
YINPSRGYTNYNQKFK

SEQ ID NO: 51 - protein, 8 aa, synthetic construct
  REGION 1..8: note = OKT3 VH-CDR3 sequence
YCARYYDD

SEQ ID NO: 52 - protein, 106 aa, synthetic construct [sequence omitted]

SEQ ID NO: 53 - protein, 11 aa, synthetic construct
  REGION 1..11: note = OKT3 VL-CDR1 sequence
SASSSVSYMNW

SEQ ID NO: 54 - protein, 7 aa, synthetic construct
  REGION 1..7: note = OKT3 VL-CDR2 sequence
TSKLASG

SEQ ID NO: 55 - protein, 9 aa, synthetic construct
  REGION 1..9: note = OKT3 VL-CDR3 sequence
QWSSNPFTF

SEQ ID NO: 56 - protein, 15 aa, synthetic construct
  REGION 1..15: note = Linker sequence
GGGGSGGGGSGGGGS

SEQ ID NO: 57 - protein, 5 aa, synthetic construct
  REGION 1..5: note = Partial linker sequence
GGGGS

SEQ ID NO: 58 - protein, 701 aa, synthetic construct
  REGION 1..701: note = BiTE molecule comprising a heavy chain sequence that includes the VH region of 1D3, a linker sequence, and the sequence of an OKT3 scFv
EVKLVESGGGLVQPGGSLRLACATSGFTFTDYYMSWVRQPPGKALEWLGFIRSKANGYTTEYSASVKGRFTISRDNSQSILYLQMNTLRSEDSATYYCARDERRTAWLAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 59 - protein, 220 aa, synthetic construct
  REGION 1..220: note = BiTE molecule comprising a light chain sequence that includes the VL region of 1D3
DVLMTQTPLSLPVSLGDQASISCRSSQSIVHSNGNTYLDWYLQKPGQSPKLLIYKVSNRFSGVPDRFSGSGSGTDFTLKISRVEAEDLGVYYCFQGSHVPTWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 114 aa, synthetic construct
  REGION 1..114: note = Core sequence of VUN100
EVQLVESGGGLVQPGGSLRLACAVSGPGLIFKFTGVAWYRRQVPGAKRGLVALITGDGATRYGDSVKGRFTVSRDIAAKRVYLEMNDLRSEDTAVYYCKTGEYWGQGTQVTVSS

SEQ ID NO: 61 - protein, 148 aa, synthetic construct
  REGION 1..148: note = VUN100 sequence with additional N-terminal signal peptide, and C-terminal Myc-6His tag
MHSSALLCCLVLLTGVRAEVQLVESGGGLVQPGGSLRLACAVSGPGLIFKFTGVAWYRRQVPGAKRGLVALITGDGATRYGDSVKGRFTVSRDIAAKRVYLEMNDLRSEDTAVYYCKTGEYWGQGTQVTVSSEQKLISEEDLHHHHHH

SEQ ID NO: 62 - protein, 258 aa, synthetic construct
  REGION 1..258: note = VUN100b
EVQLVESGGGLVQPGGSLRLACAVSGPGLIFKFTGVAWYRRQVPGAKRGLVALITGDGATRYGDSVKGRFTVSRDIAAKRVYLEMNDLRSEDTAVYYCKTGEYWGQGTQVTVSSGGGGSGGGGSGGGGSGGGGSGGGGSGGGGSEVQLVESGGGLVQPGGSLRLACAVSGPGLIFKFTGVAWYRRQVPGAKRGLVALITGDGATRYGDSVKGRFTVSRDIAAKRVYLEMNDLRSEDTAVYYCKTGEYWGQGTQVTVSS

SEQ ID NO: 63 - protein, 292 aa, synthetic construct
  REGION 1..292: note = VUN100b with additional N-terminal signal peptide, and C-terminal Myc-6His tag
MHSSALLCCLVLLTGVRAEVQLVESGGGLVQPGGSLRLACAVSGPGLIFKFTGVAWYRRQVPGAKRGLVALITGDGATRYGDSVKGRFTVSRDIAAKRVYLEMNDLRSEDTAVYYCKTGEYWGQGTQVTVSSGGGGSGGGGSGGGGSGGGGSGGGGSGGGGSEVQLVESGGGLVQPGGSLRLACAVSGPGLIFKFTGVAWYRRQVPGAKRGLVALITGDGATRYGDSVKGRFTVSRDIAAKRVYLEMNDLRSEDTAVYYCKTGEYWGQGTQVTVSSEQKLISEEDLHHHHHH

SEQ ID NO: 64 - skipped

SEQ ID NO: 65 - DNA, 423 bp, synthetic construct
  misc_feature 1..423: note = Immature form of the recombinantly expressed VH sequence of 1G9
  source 1..423: mol_type = other DNA
atgaagttgtggctgaactggattttccttgtaacacttttaaatggtatccagtgtgaagtgaagctggtggagtctgggggaggcttagtgaagcctggagggtccctgaaactctcctgtgcagcctctggattcactttcagtagctatgccatgtcttgggttcgccagactccagagaagaggctggagtgggtcgcatccattagtagtggtggtagcacctactatccagacagtgtgaagggccgattcaccatctccagagataatgccaggaacatcctgtacctgcaaatgagcagtctgaggtctgaggacacggccatgtattactgtgcaagaggcgggtctactatgattacgacggggctggggtttgcttactggggccaagggactctggtcactgtctctgca

SEQ ID NO: 66 - DNA, 366 bp, synthetic construct
  misc_feature 1..366: note = Mature form of VH sequence of 1G9
  source 1..366: mol_type = other DNA
gaagtgaagctggtggagtctgggggaggcttagtgaagcctggagggtccctgaaactctcctgtgcagcctctggattcactttcagtagctatgccatgtcttgggttcgccagactccagagaagaggctggagtgggtcgcatccattagtagtggtggtagcacctactatccagacagtgtgaagggccgattcaccatctccagagataatgccaggaacatcctgtacctgcaaatgagcagtctgaggtctgaggacacggccatgtattactgtgcaagaggcgggtctactatgattacgacggggctggggtttgcttactggggccaagggactctggtcactgtctctgca

SEQ ID NO: 67 - protein, 141 aa, synthetic construct
  REGION 1..141: note = Immature form of the recombinantly expressed VH sequence of 1G9, wherein positions 1-19 correspond to a leader sequence
MKLWLNWIFLVTLLNGIQCEVKLVESGGGLVKPGGSLKLSCAASGFTFSSYAMSWVRQTPEKRLEWVASISSGGSTYYPDSVKGRFTISRDNARNILYLQMSSLRSEDTAMYYCARGGSTMITTGLGFAYWGQGTLVTVSA

SEQ ID NO: 68 - protein, 122 aa, synthetic construct
  REGION 1..122: note = Mature form of VH sequence of 1G9, without the leader sequence
EVKLVESGGGLVKPGGSLKLSCAASGFTFSSYAMSWVRQTPEKRLEWVASISSGGSTYYPDSVKGRFTISRDNARNILYLQMSSLRSEDTAMYYCARGGSTMITTGLGFAYWGQGTLVTVSA

SEQ ID NO: 69 - DNA, 378 bp, synthetic construct
  misc_feature 1..378: note = Immature form of the recombinantly expressed VL sequence of 1G9
  source 1..378: mol_type = other DNA
atgaagttgcctgttaggctgttggtgctgatggtctggattcctgcttccagcagtcaaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccacccctcacgttcggtgctgggaccaagctggagctgaaa

SEQ ID NO: 70 - DNA, 321 bp, synthetic construct
  misc_feature 1..321: note = Mature form of VL sequence of 1G9
  source 1..321: mol_type = other DNA
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccacccctcacgttcggtgctgggaccaagctggagctgaaa

SEQ ID NO: 71 - protein, 126 aa, synthetic construct
  REGION 1..126: note = Immature form of the recombinantly expressed VL sequence of 1G9, wherein positions 1-19 correspond to a leader sequence
MKLPVRLLVLMVWIPASSSQIVLTQSPAIMSASPGEKVTMTCSASSSVSYMHWYQQKSGTSPKRWIYDTSKLASGVPARFSGSGSGTSYSLTISSMEAEDAATYYCQQWSSNPPLTFGAGTKLELK

SEQ ID NO: 72 - protein, 107 aa, synthetic construct
  REGION 1..107: note = Mature form of VL sequence of 1G9, without the leader sequence
QIVLTQSPAIMSASPGEKVTMTCSASSSVSYMHWYQQKSGTSPKRWIYDTSKLASGVPARFSGSGSGTSYSLTISSMEAEDAATYYCQQWSSNPPLTFGAGTKLELK

SEQ ID NO: 73 - DNA, 15 bp, synthetic construct
  misc_feature 1..15: note = VH-CDR1 sequence of 1G9, 1E8
  source 1..15: mol_type = other DNA
agctatgccatgtct

SEQ ID NO: 74 - DNA, 48 bp, synthetic construct
  misc_feature 1..48: note = VH-CDR2 sequence of 1G9
  source 1..48: mol_type = other DNA
tccattagtagtggtggtagcacctactatccagacagtgtgaagggc

SEQ ID NO: 75 - DNA, 42 bp, synthetic construct
  misc_feature 1..42: note = VH-CDR3 sequence of 1G9
  source 1..42: mol_type = other DNA
ggcgggtctactatgattacgacggggctggggtttgcttac

SEQ ID NO: 76 - protein, 5 aa, synthetic construct
  REGION 1..5: note = VH-CDR1 sequence of 1G9, 1E8
SYAMS

SEQ ID NO: 77 - protein, 16 aa, synthetic construct
  REGION 1..16: note = VH-CDR2 sequence of 1G9
SISSGGSTYYPDSVKG

SEQ ID NO: 78 - protein, 14 aa, synthetic construct
  REGION 1..14: note = VH-CDR3 sequence of 1G9
GGSTMITTGLGFAY

SEQ ID NO: 79 - DNA, 30 bp, synthetic construct
  misc_feature 1..30: note = VL-CDR1 sequence of 1G9, 1E8
  source 1..30: mol_type = other DNA
agtgccagctcaagtgtaagttacatgcac

SEQ ID NO: 80 - DNA, 21 bp, synthetic construct
  misc_feature 1..21: note = VL-CDR2 sequence of 1G9, 1C10, 1A10
  source 1..21: mol_type = other DNA
gacacatccaaactggcttct

SEQ ID NO: 81 - DNA, 30 bp, synthetic construct
  misc_feature 1..30: note = VL-CDR3 sequence of 1G9
  source 1..30: mol_type = other DNA
cagcagtggagtagtaacccacccctcacg

SEQ ID NO: 82 - protein, 10 aa, synthetic construct
  REGION 1..10: note = VL-CDR1 sequence of 1G9, 1E8
SASSSVSYMH

SEQ ID NO: 83 - protein, 7 aa, synthetic construct
  REGION 1..7: note = VL-CDR2 sequence of 1G9, 1C10, 1A10
DTSKLAS

SEQ ID NO: 84 - protein, 10 aa, synthetic construct
  REGION 1..10: note = VL-CDR3 sequence of 1G9
QQWSSNPPLT

SEQ ID NO: 85 - DNA, 420 bp, synthetic construct
  misc_feature 1..420: note = Immature form of the recombinantly expressed VH sequence of 1E8
  source 1..420: mol_type = other DNA
atgaagttgtggctgaactggattttccttgtaacacttttaaatggtatccagtgtgaagtgaagctggtggagtctgggggagacttagtgaagcctggagggtccctgaaactctcctgtgcagcctctggattcactttcagtagctatgccatgtcttgggttcgccagactccagagaagaggctggagtgggtcgcatccattagtagtggtggtagaacctactatccagacagtgtgaagggccgattcaccatctccagagataatgccaggaacatcctgtacctgcaaatgagcagtctgaggtctgaagacacggccatctattactgtgcacgaggcgggactcgtcattcctacggcaacgggtttgactactggggccaaggcaccactctcacagtctcctca

SEQ ID NO: 86 - DNA, 363 bp, synthetic construct
  misc_feature 1..363: note = Mature form of VH sequence of 1E8
  source 1..363: mol_type = other DNA
gaagtgaagctggtggagtctgggggagacttagtgaagcctggagggtccctgaaactctcctgtgcagcctctggattcactttcagtagctatgccatgtcttgggttcgccagactccagagaagaggctggagtgggtcgcatccattagtagtggtggtagaacctactatccagacagtgtgaagggccgattcaccatctccagagataatgccaggaacatcctgtacctgcaaatgagcagtctgaggtctgaagacacggccatctattactgtgcacgaggcgggactcgtcattcctacggcaacgggtttgactactggggccaaggcaccactctcacagtctcctca

SEQ ID NO: 87 - protein, 140 aa, synthetic construct
  REGION 1..140: note = Immature form of the recombinantly expressed VH sequence of 1E8, wherein positions 1-19 correspond to a leader sequence
MKLWLNWIFLVTLLNGIQCEVKLVESGGDLVKPGGSLKLSCAASGFTFSSYAMSWVRQTPEKRLEWVASISSGGRTYYPDSVKGRFTISRDNARNILYLQMSSLRSEDTAIYYCARGGTRHSYGNGFDYWGQGTTLTVSS

SEQ ID NO: 88 - protein, 121 aa, synthetic construct
  REGION 1..121: note = Mature form of VH sequence of 1E8, without the leader sequence
EVKLVESGGDLVKPGGSLKLSCAASGFTFSSYAMSWVRQTPEKRLEWVASISSGGRTYYPDSVKGRFTISRDNARNILYLQMSSLRSEDTAIYYCARGGTRHSYGNGFDYWGQGTTLTVSS

SEQ ID NO: 89 - DNA, 378 bp, synthetic construct
  misc_feature 1..378: note = Immature form of the recombinantly expressed VL sequence of 1E8
  source 1..378: mol_type = other DNA
atgaagttgcctgttaggctgttggtgctgatggtctggattcctgcttccagcagtcaaattgttctctcccagtctccaacaatcatgtctgcatctccaggggagaaggtcaccatgacctgcagtgccagctcaagtgtaagttacatgcactggtaccagcagaagtcaggcacctcccccaaaagatggatttatgactcatccaaactggcttctggagtccctgctcgcttcagtggcagtgggtctgggacctcttactctctcacaatcagcagcatggaggctgaagatgctgccacttattactgccagcagtggactagtaacccacccatcacgttcggtgctgggaccaagctggagctgaaa

SEQ ID NO: 90 - DNA, 321 bp, synthetic construct
  misc_feature 1..321: note = Mature form of VL sequence of 1E8
  source 1..321: mol_type = other DNA
caaattgttctctcccagtctccaacaatcatgtctgcatctccaggggagaaggtcaccatgacctgcagtgccagctcaagtgtaagttacatgcactggtaccagcagaagtcaggcacctcccccaaaagatggatttatgactcatccaaactggcttctggagtccctgctcgcttcagtggcagtgggtctgggacctcttactctctcacaatcagcagcatggaggctgaagatgctgccacttattactgccagcagtggactagtaacccacccatcacgttcggtgctgggaccaagctggagctgaaa

SEQ ID NO: 91 - protein, 126 aa, synthetic construct
  REGION 1..126: note = Immature form of the recombinantly expressed VL sequence of 1E8, wherein positions 1-19 correspond to a leader sequence
MKLPVRLLVLMVWIPASSSQIVLSQSPTIMSASPGEKVTMTCSASSSVSYMHWYQQKSGTSPKRWIYDSSKLASGVPARFSGSGSGTSYSLTISSMEAEDAATYYCQQWTSNPPITFGAGTKLELK

SEQ ID NO: 92 - protein, 107 aa, synthetic construct
  REGION 1..107: note = Mature form of VL sequence of 1E8, without the leader sequence
QIVLSQSPTIMSASPGEKVTMTCSASSSVSYMHWYQQKSGTSPKRWIYDSSKLASGVPARFSGSGSGTSYSLTISSMEAEDAATYYCQQWTSNPPITFGAGTKLELK

SEQ ID NO: 93 - DNA, 48 bp, synthetic construct
  misc_feature 1..48: note = VH-CDR2 sequence of 1E8
  source 1..48: mol_type = other DNA
tccattagtagtggtggtagaacctactatccagacagtgtgaagggc

SEQ ID NO: 94 - DNA, 39 bp, synthetic construct
  misc_feature 1..39: note = VH-CDR3 sequence of 1E8
  source 1..39: mol_type = other DNA
ggcgggactcgtcattcctacggcaacgggtttgactac

SEQ ID NO: 95 - protein, 16 aa, synthetic construct
  REGION 1..16: note = VH-CDR2 sequence of 1E8
SISSGGRTYYPDSVKG

SEQ ID NO: 96 - protein, 13 aa, synthetic construct
  REGION 1..13: note = VH-CDR3 sequence of 1E8
GGTRHSYGNGFDY

SEQ ID NO: 97 - DNA, 21 bp, synthetic construct
  misc_feature 1..21: note = VL-CDR2 sequence of 1E8
  source 1..21: mol_type = other DNA
gactcatccaaactggcttct

SEQ ID NO: 98 - DNA, 30 bp, synthetic construct
  misc_feature 1..30: note = VL-CDR3 sequence of 1E8
  source 1..30: mol_type = other DNA
cagcagtggactagtaacccacccatcacg

SEQ ID NO: 99 - protein, 7 aa, synthetic construct
  REGION 1..7: note = VL-CDR2 sequence of 1E8
DSSKLAS

SEQ ID NO: 100 - protein, 10 aa, synthetic construct
  REGION 1..10: note = VL-CDR3 sequence of 1E8
QQWTSNPPIT

SEQ ID NO: 101 - DNA, 420 bp, synthetic construct
  misc_feature 1..420: note = Immature form of the recombinantly expressed VH sequence of 1C10
  source 1..420: mol_type = other DNA
atgaagttgtggctgaactggattttccttgtaacacttttaaatggtatccagtgtgaagtgaaactggttgagtctgggggagtcttagtgaagcctggagggtccctgaaattttcctgtgcagcctctggattcactttcagtagccatgccttgtcttgggttcgccagactccagagaagagactggagtgggtcgcatccattagtagtcgtggtcgaacctactatccagacagtgtaaagggccgattcaccgtctccagagataatgccaggaacatcctgtatctgcaagtgagcagtctgaggtctgaggacacggccatgtattattgtacaagaggcgggactcattattcctacggcaacggttttgacttctggggccaaggcaccactctcacagtctcctca

SEQ ID NO: 102 - DNA, 363 bp, synthetic construct
  misc_feature 1..363: note = Mature form of VH sequence of 1C10
  source 1..363: mol_type = other DNA
gaagtgaaactggttgagtctgggggagtcttagtgaagcctggagggtccctgaaattttcctgtgcagcctctggattcactttcagtagccatgccttgtcttgggttcgccagactccagagaagagactggagtgggtcgcatccattagtagtcgtggtcgaacctactatccagacagtgtaaagggccgattcaccgtctccagagataatgccaggaacatcctgtatctgcaagtgagcagtctgaggtctgaggacacggccatgtattattgtacaagaggcgggactcattattcctacggcaacggttttgacttctggggccaaggcaccactctcacagtctcctca

SEQ ID NO: 103 - protein, 140 aa, synthetic construct
  REGION 1..140: note = Immature form of the recombinantly expressed VH sequence of 1C10, wherein positions 1-19 correspond to a leader sequence
MKLWLNWIFLVTLLNGIQCEVKLVESGGVLVKPGGSLKFSCAASGFTFSSHALSWVRQTPEKRLEWVASISSRGRTYYPDSVKGRFTVSRDNARNILYLQVSSLRSEDTAMYYCTRGGTHYSYGNGFDFWGQGTTLTVSS

SEQ ID NO: 104 - protein, 121 aa, synthetic construct
  REGION 1..121: note = Mature form of VH sequence of 1C10, without the leader sequence
EVKLVESGGVLVKPGGSLKFSCAASGFTFSSHALSWVRQTPEKRLEWVASISSRGRTYYPDSVKGRFTVSRDNARNILYLQVSSLRSEDTAMYYCTRGGTHYSYGNGFDFWGQGTTLTVSS

SEQ ID NO: 105 - DNA, 378 bp, synthetic construct
  misc_feature 1..378: note = Immature form of the recombinantly expressed VL sequence of 1C10
  source 1..378: mol_type = other DNA
atgaagttgcctgttaggctgttggtgctgatggtctggattcctgcttccagcagtcaaattgttctcacccagtctccagcaatcatgtctgcatctccaggggagaaggtcaccatgacctgcagtgtcagctcaagtgttagttacatgcactggtaccagcagaagtcaggcacctcccccaaaagatggatttatgacacatccaaactggcttctggagtccctgctcgcttcagtggcagtgggtctgggacctcttactctctcacaatcagcaccatggaggctgaagatgctgccacttattactgccagcagtggagtaataacccacccatcacgttcggtgctgggaccaagctggaactgaaa

SEQ ID NO: 106 - DNA, 321 bp, synthetic construct
  misc_feature 1..321: note = Mature form of VL sequence of 1C10
  source 1..321: mol_type = other DNA
caaattgttctcacccagtctccagcaatcatgtctgcatctccaggggagaaggtcaccatgacctgcagtgtcagctcaagtgttagttacatgcactggtaccagcagaagtcaggcacctcccccaaaagatggatttatgacacatccaaactggcttctggagtccctgctcgcttcagtggcagtgggtctgggacctcttactctctcacaatcagcaccatggaggctgaagatgctgccacttattactgccagcagtggagtaataacccacccatcacgttcggtgctgggaccaagctggaactgaaa

SEQ ID NO: 107 - protein, 126 aa, synthetic construct
  REGION 1..126: note = Immature form of the recombinantly expressed VL sequence of 1C10, wherein positions 1-19 correspond to a leader sequence
MKLPVRLLVLMVWIPASSSQIVLTQSPAIMSASPGEKVTMTCSVSSSVSYMHWYQQKSGTSPKRWIYDTSKLASGVPARFSGSGSGTSYSLTISTMEAEDAATYYCQQWSNNPPITFGAGTKLELK

SEQ ID NO: 108 - protein, 107 aa, synthetic construct
  REGION 1..107: note = Mature form of VL sequence of 1C10, without the leader sequence
QIVLTQSPAIMSASPGEKVTMTCSVSSSVSYMHWYQQKSGTSPKRWIYDTSKLASGVPARFSGSGSGTSYSLTISTMEAEDAATYYCQQWSNNPPITFGAGTKLELK

SEQ ID NO: 109 - DNA, 15 bp, synthetic construct
  misc_feature 1..15: note = VH-CDR1 sequence of 1C10, 1A10
  source 1..15: mol_type = other DNA
agccatgccttgtct

SEQ ID NO: 110 - DNA, 48 bp, synthetic construct
  misc_feature 1..48: note = VH-CDR2 sequence of 1C10, 1A10
  source 1..48: mol_type = other DNA
tccattagtagtcgtggtcgaacctactatccagacagtgtaaagggc

SEQ ID NO: 111 - DNA, 39 bp, synthetic construct
  misc_feature 1..39: note = VH-CDR3 sequence of 1C10, 1A10
  source 1..39: mol_type = other DNA
ggcgggactcattattcctacggcaacggttttgacttc

SEQ ID NO: 112 - protein, 5 aa, synthetic construct
  REGION 1..5: note = VH-CDR1 sequence of 1C10, 1A10
SHALS

SEQ ID NO: 113 - protein, 16 aa, synthetic construct
  REGION 1..16: note = VH-CDR2 sequence of 1C10, 1A10
SISSRGRTYYPDSVKG

SEQ ID NO: 114 - protein, 13 aa, synthetic construct
  REGION 1..13: note = VH-CDR3 sequence of 1C10, 1A10
GGTHYSYGNGFDF

SEQ ID NO: 115 - DNA, 30 bp, synthetic construct
  misc_feature 1..30: note = VL-CDR1 sequence of 1C10, 1A10
  source 1..30: mol_type = other DNA
agtgtcagctcaagtgttagttacatgcac

SEQ ID NO: 116 - DNA, 30 bp, synthetic construct
  misc_feature 1..30: note = VL-CDR3 sequence of 1C10, 1A10
  source 1..30: mol_type = other DNA
cagcagtggagtaataacccacccatcacg

SEQ ID NO: 117 - protein, 10 aa, synthetic construct
  REGION 1..10: note = VL-CDR1 sequence of 1C10, 1A10
SVSSSVSYMH

SEQ ID NO: 118 - protein, 10 aa, synthetic construct
  REGION 1..10: note = VL-CDR3 sequence of 1C10, 1A10
QQWSNNPPIT

SEQ ID NO: 119 - DNA, 420 bp, synthetic construct
  misc_feature 1..420: note = Immature form of the recombinantly expressed VH sequence of 1A10
  source 1..420: mol_type = other DNA
atgaagttgtggctgaactggattttccttgtaacacttttaaatggtatccagtgtgaagtgaaactggttgagtctgggggagtcttagtgaagcctggagggtccctgaaattttcctgtgcagcctctggattcactctcagtagccatgccttgtcttgggttcgccagactccagagaagagactggagtgggtcgcatccattagtagtcgtggtcgaacctactatccagacagtgtaaagggccgattcaccgtctccagagataatgccaggaacatcctgtatctgcaagtgagcagtctgaggtctgaggacacggccatgtattattgtacaagaggcgggactcattattcctacggcaacggttttgacttctggggccaaggcaccactctcacagtctcctca

SEQ ID NO: 120 - DNA, 363 bp, synthetic construct
  misc_feature 1..363: note = Mature form of VH sequence of 1A10
  source 1..363: mol_type = other DNA
gaagtgaaactggttgagtctgggggagtcttagtgaagcctggagggtccctgaaattttcctgtgcagcctctggattcactctcagtagccatgccttgtcttgggttcgccagactccagagaagagactggagtgggtcgcatccattagtagtcgtggtcgaacctactatccagacagtgtaaagggccgattcaccgtctccagagataatgccaggaacatcctgtatctgcaagtgagcagtctgaggtctgaggacacggccatgtattattgtacaagaggcgggactcattattcctacggcaacggttttgacttctggggccaaggcaccactctcacagtctcctca

SEQ ID NO: 121 - protein, 140 aa, synthetic construct
  REGION 1..140: note = Immature form of the recombinantly expressed VH sequence of 1A10, wherein positions 1-19 correspond to a leader sequence
MKLWLNWIFLVTLLNGIQCEVKLVESGGVLVKPGGSLKFSCAASGFTLSSHALSWVRQTPEKRLEWVASISSRGRTYYPDSVKGRFTVSRDNARNILYLQVSSLRSEDTAMYYCTRGGTHYSYGNGFDFWGQGTTLTVSS

SEQ ID NO: 122 - protein, 121 aa, synthetic construct
  REGION 1..121: note = Mature form of VH sequence of 1A10, without the leader sequence
EVKLVESGGVLVKPGGSLKFSCAASGFTLSSHALSWVRQTPEKRLEWVASISSRGRTYYPDSVKGRFTVSRDNARNILYLQVSSLRSEDTAMYYCTRGGTHYSYGNGFDFWGQGTTLTVSS

SEQ ID NO: 123 - DNA, 378 bp, synthetic construct; identical to SEQ ID NO: 105
  misc_feature 1..378: note = Immature form of the recombinantly expressed VL sequence of 1A10
  source 1..378: mol_type = other DNA

SEQ ID NO: 124 - DNA, 321 bp, synthetic construct; identical to SEQ ID NO: 106
  misc_feature 1..321: note = Mature form of VL sequence of 1A10
  source 1..321: mol_type = other DNA

SEQ ID NO: 125 - protein, 126 aa, synthetic construct; identical to SEQ ID NO: 107
  REGION 1..126: note = Immature form of the recombinantly expressed VL sequence of 1A10, wherein positions 1-19 correspond to a leader sequence

SEQ ID NO: 126 - protein, 107 aa, synthetic construct; identical to SEQ ID NO: 108
  REGION 1..107: note = Mature form of VL sequence of 1A10, without the leader sequence

SEQ ID NO: 127 - protein, 354 aa, synthetic construct
  REGION 1..354: note = Full sequence of US28 as encoded by HCMV strain BL (Accession number MW980585.1)
MTPTTTTAELTTEFDYDEDAAPCVLTDVLNQSKPVTLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNVELMLGAFVIPLSVISYCYYRISRIVAVSQSRHKGRIVRVLIAVVLVFIIFWLPYHLTLFLDTLKLLKWISSSCEFEKSLKRALILTESLAFCHCCLNPLLYVFVGTKFRQELHCLLAEFRQRLFSRDVSWYHSMSFSRRSSPSRRETSSDTLSDEACRVSQIIP

SEQ ID NO: 128-146 - skipped (19 entries)

SEQ ID NO: 147 - DNA, 1396 bp, synthetic construct
  misc_feature 1..1396: note = Complete sequence of the heavy chain of 1G9
  source 1..1396: mol_type = other DNA
atgaagttgtggctgaactggattttccttgtaacacttttaaatggtatccagtgtgaagtgaagctggtggagtctgggggaggcttagtgaagcctggagggtccctgaaactctcctgtgcagcctctggattcactttcagtagctatgccatgtcttgggttcgccagactccagagaagaggctggagtgggtcgcatccattagtagtggtggtagcacctactatccagacagtgtgaagggccgattcaccatctccagagataatgccaggaacatcctgtacctgcaaatgagcagtctgaggtctgaggacacggccatgtattactgtgcaagaggcgggtctactatgattacgacggggctggggtttgcttactggggccaagggactctggtcactgtctctgcacaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 148 - DNA, 702 bp, synthetic construct
  misc_feature 1..702: note = Complete sequence of the light chain of 1G9
  source 1..702: mol_type = other DNA
atgaagttgcctgttaggctgttggtgctgatggtctggattcctgcttccagcagtcaaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccacccctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 149 - protein, 465 aa, synthetic construct
  REGION 1..465: note = Complete sequence of the heavy chain of 1G9
MKLWLNWIFLVTLLNGIQCEVKLVESGGGLVKPGGSLKLSCAASGFTFSSYAMSWVRQTPEKRLEWVASISSGGSTYYPDSVKGRFTISRDNARNILYLQMSSLRSEDTAMYYCARGGSTMITTGLGFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 150 - protein, 233 aa, synthetic construct
  REGION 1..233: note = Complete sequence of the light chain of 1G9
MKLPVRLLVLMVWIPASSSQIVLTQSPAIMSASPGEKVTMTCSASSSVSYMHWYQQKSGTSPKRWIYDTSKLASGVPARFSGSGSGTSYSLTISSMEAEDAATYYCQQWSSNPPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 151 - DNA, 1393 bp, synthetic construct
  misc_feature 1..1393: note = Complete sequence of the heavy chain of 1E8
  source 1..1393: mol_type = other DNA
atgaagttgtggctgaactggattttccttgtaacacttttaaatggtatccagtgtgaagtgaagctggtggagtctgggggagacttagtgaagcctggagggtccctgaaactctcctgtgcagcctctggattcactttcagtagctatgccatgtcttgggttcgccagactccagagaagaggctggagtgggtcgcatccattagtagtggtggtagaacctactatccagacagtgtgaagggccgattcaccatctccagagataatgccaggaacatcctgtacctgcaaatgagcagtctgaggtctgaagacacggccatctattactgtgcacgaggcgggactcgtcattcctacggcaacgggtttgactactggggccaaggcaccactctcacagtctcctcacaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 152 - DNA, 702 bp, synthetic construct
  misc_feature 1..702: note = Complete sequence of the light chain of 1E8
  source 1..702: mol_type = other DNA
atgaagttgcctgttaggctgttggtgctgatggtctggattcctgcttccagcagtcaaattgttctctcccagtctccaacaatcatgtctgcatctccaggggagaaggtcaccatgacctgcagtgccagctcaagtgtaagttacatgcactggtaccagcagaagtcaggcacctcccccaaaagatggatttatgactcatccaaactggcttctggagtccctgctcgcttcagtggcagtgggtctgggacctcttactctctcacaatcagcagcatggaggctgaagatgctgccacttattactgccagcagtggactagtaacccacccatcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 153 - protein, 464 aa, synthetic construct
  REGION 1..464: note = Complete sequence of the heavy chain of 1E8
MKLWLNWIFLVTLLNGIQCEVKLVESGGDLVKPGGSLKLSCAASGFTFSSYAMSWVRQTPEKRLEWVASISSGGRTYYPDSVKGRFTISRDNARNILYLQMSSLRSEDTAIYYCARGGTRHSYGNGFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 154 - protein, 233 aa, synthetic construct
  REGION 1..233: note = Complete sequence of the light chain of 1E8
MKLPVRLLVLMVWIPASSSQIVLSQSPTIMSASPGEKVTMTCSASSSVSYMHWYQQKSGTSPKRWIYDSSKLASGVPARFSGSGSGTSYSLTISSMEAEDAATYYCQQWTSNPPITFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 155 - DNA, 1393 bp, synthetic construct
  misc_feature 1..1393: note = Complete sequence of the heavy chain of 1C10
  source 1..1393: mol_type = other DNA
atgaagttgtggctgaactggattttccttgtaacacttttaaatggtatccagtgtgaagtgaaactggttgagtctgggggagtcttagtgaagcctggagggtccctgaaattttcctgtgcagcctctggattcactttcagtagccatgccttgtcttgggttcgccagactccagagaagagactggagtgggtcgcatccattagtagtcgtggtcgaacctactatccagacagtgtaaagggccgattcaccgtctccagagataatgccaggaacatcctgtatctgcaagtgagcagtctgaggtctgaggacacggccatgtattattgtacaagaggcgggactcattattcctacggcaacggttttgacttctggggccaaggcaccactctcacagtctcctcacaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 156 - DNA, 702 bp, synthetic construct
  misc_feature 1..702: note = Complete sequence of the light chain of 1C10
  source 1..702: mol_type = other DNA
atgaagttgcctgttaggctgttggtgctgatggtctggattcctgcttccagcagtcaaattgttctcacccagtctccagcaatcatgtctgcatctccaggggagaaggtcaccatgacctgcagtgtcagctcaagtgttagttacatgcactggtaccagcagaagtcaggcacctcccccaaaagatggatttatgacacatccaaactggcttctggagtccctgctcgcttcagtggcagtgggtctgggacctcttactctctcacaatcagcaccatggaggctgaagatgctgccacttattactgccagcagtggagtaataacccacccatcacgttcggtgctgggaccaagctggaactgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 157 - protein, 464 aa, synthetic construct
  REGION 1..464: note = Complete sequence of the heavy chain of 1C10
MKLWLNWIFLVTLLNGIQCEVKLVESGGVLVKPGGSLKFSCAASGFTFSSHALSWVRQTPEKRLEWVASISSRGRTYYPDSVKGRFTVSRDNARNILYLQVSSLRSEDTAMYYCTRGGTHYSYGNGFDFWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 158 - protein, 233 aa, synthetic construct
  REGION 1..233: note = Complete sequence of the light chain of 1C10
MKLPVRLLVLMVWIPASSSQIVLTQSPAIMSASPGEKVTMTCSVSSSVSYMHWYQQKSGTSPKRWIYDTSKLASGVPARFSGSGSGTSYSLTISTMEAEDAATYYCQQWSNNPPITFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 159 - DNA, 1393 bp, synthetic construct
  misc_feature 1..1393: note = Complete sequence of the heavy chain of 1A10
  source 1..1393: mol_type = other DNA
atgaagttgtggctgaactggattttccttgtaacacttttaaatggtatccagtgtgaagtgaaactggttgagtctgggggagtcttagtgaagcctggagggtccctgaaattttcctgtgcagcctctggattcactctcagtagccatgccttgtcttgggttcgccagactccagagaagagactggagtgggtcgcatccattagtagtcgtggtcgaacctactatccagacagtgtaaagggccgattcaccgtctccagagataatgccaggaacatcctgtatctgcaagtgagcagtctgaggtctgaggacacggccatgtattattgtacaagaggcgggactcattattcctacggcaacggttttgacttctggggccaaggcaccactctcacagtctcctcacaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 160 - DNA, 702 bp, synthetic construct; identical to SEQ ID NO: 156
  misc_feature 1..702: note = Complete sequence of the light chain of 1A10
  source 1..702: mol_type = other DNA

SEQ ID NO: 161 - protein, 464 aa, synthetic construct
  REGION 1..464: note = Complete sequence of the heavy chain of 1A10
MKLWLNWIFLVTLLNGIQCEVKLVESGGVLVKPGGSLKFSCAASGFTLSSHALSWVRQTPEKRLEWVASISSRGRTYYPDSVKGRFTVSRDNARNILYLQVSSLRSEDTAMYYCTRGGTHYSYGNGFDFWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 162 - protein, 233 aa, synthetic construct; identical to SEQ ID NO: 158
  REGION 1..233: note = Complete sequence of the light chain of 1A10

SEQ ID NO: 163-167 - skipped (5 entries)

SEQ ID NO: 168 - protein, 16 aa, synthetic construct
  REGION 1..16: note = X5 = G or R; X7 = S or R
  VARIANT 5: note = G or R
  VARIANT 7: note = S or R
SISSXGXTYYPDSVKG

SEQ ID NO: 169 - protein, 14 aa, synthetic construct
  REGION 1..14: note = X3 = S or T; X4 = T, R or H; X5 = M, H or Y; X6 = I or S; X7 = T or Y; X8 = T or G; X9 = G or N; X10 = L or blank; X13 = A or D; X14 = Y or F
  VARIANT 3: note = S or T
  VARIANT 4: note = T, R or H
  VARIANT 5: note = M, H or Y
  VARIANT 6: note = I or S
  VARIANT 7: note = T or Y
  VARIANT 8: note = T or G
  VARIANT 9: note = G or N
  VARIANT 10: note = L or blank (also denoted as *)
  VARIANT 13: note = A or D
  VARIANT 14: note = Y or F
GGXXXXXXXXGFXX

SEQ ID NO: 170 - protein, 10 aa, synthetic construct
  REGION 1..10: note = X2 = A or V
  VARIANT 2: note = A or V
SXSSSVSYMH

SEQ ID NO: 171 - protein, 7 aa, synthetic construct
  REGION 1..7: note = X2 = T or S
  VARIANT 2: note = T or S
DXSKLAS

SEQ ID NO: 172 - protein, 11 aa, synthetic construct
  REGION 1..11: note = X4 = S, T or blank; X7 = blank or N; X10 = I or L
  VARIANT 4: note = S, T or blank (also denoted as *)
  VARIANT 7: note = N or blank (also denoted as *)
  VARIANT 10: note = I or L
QQWXSNXPPXT

SEQ ID NO: 173 - protein, 353 aa, synthetic construct
  REGION 1..353: note = Artifically mutated strain of US28
MTQTTTTELTTEFDYDLGAALCTLTDVLNQSKPITLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNVELMLGAFVIPLSVISYCYYRISRIVAVSQSRHKGRIVRVLIAVVLVFIIFWLPYHLTLFLDTLKLLKWISSSCESEKSLKRALILTESLAFCHCCLNPLLYVFVGTKFRQELHCLLAEFRQRLFSRDVSWYHSMSFSRRSSPSRRETSSDTLSDEVCRVSQIIP

SEQ ID NO: 174 - protein, 16 aa, synthetic construct
  REGION 1..16: note = Wherein X = G or R
  VARIANT 5: note = G or R
SISSXGRTYYPDSVKG

SEQ ID NO: 175 - protein, 13 aa, synthetic construct
  REGION 1..13: note = X3 = S or T; X4 = T, R or H; X5 = M, H or Y; X6 = I or S; X7 = T or Y; X8 = T or G; X9 = G or N; X12 = A or D; X13 = Y or F
  VARIANT 3: note = S or T
  VARIANT 4: note = T, R or H
  VARIANT 5: note = M, H or Y
  VARIANT 6: note = I or S
  VARIANT 7: note = T or Y
  VARIANT 8: note = T or G
  VARIANT 9: note = G or N
  VARIANT 12: note = A or D
  VARIANT 13: note = Y or F
GGXXXXXXXGFXX

SEQ ID NO: 176 - protein, 11 aa, synthetic construct
  REGION 1..11: note = X4 = T or blank; X7 = blank or N; X10 = I or L
  VARIANT 4: note = T or blank
  VARIANT 7: note = N or blank
  VARIANT 10: note = I or L
QQWXSNXPPXT

SEQ ID NO: 177 - protein, 12 aa, synthetic construct
  REGION 1..12: note = ECD1 of the US28 protein used as Peptide-N
TDVLNQSKPVTL

SEQ ID NO: 178 - DNA, 1431 bp, synthetic construct
  misc_feature 1..1431: note = Complete sequence of the heavy chain of 4H3C3
  source 1..1431: mol_type = other DNA
gcggccgcaaactacaagacagacttgcaaaagaaggcatgcacagctcagcactgctctgttgcctggtcctcctgactggggtgagggcccaggtgcagctgcagcagtctggagctgaactggctaaacccggcgcttctgtgaaaatgtcttgtaaagcctcaggctataccttcacatcctactggatgcactgggtgaaacagagacccggccagggactggaatggattggatacatcaacccctcaaccggctacgccgagtataaccagaaattcaaggacaaggctaccctgaccgccgacaaaagcagctcaaccgcttacatgcagctgagcagcctgaccagcgaggattctgctgtgtactactgcgccagactgagattcggcagcagcggattcgcctactggggacagggaacactggtgacagtgtcttccgctaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccgcaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatgattctaga

SEQ ID NO: 179 - DNA, 762 bp, synthetic construct
  misc_feature 1..762: note = Complete sequence of the light chain of 4H3C3
  source 1..762: mol_type = other DNA
gcggccgcaaactacaagacagacttgcaaaagaaggcatgcacagctcagcactgctctgttgcctggtcctcctgactggggtgagggcccagatcgtgctgacccagtcaccaagcagcctggctgtgtctgtgggagaaaaggtgaccatgagctgcaaaagctcacagagcctgctgtacagctccaaccagaagaactacctggcctggtatcagcagaaacccggccagtcacccaaactgctgatctattgggccagcacctgggagagcggagtgcctgatagattcaccgggtccggaagcggaacagacttcacactgaccattagcagcgtgaaagctgaggacctggccgtgtactactgccagcagtactacagctttcccctgacctttggagccggcacaaagctggagatcaagagagcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgattctaga

SEQ ID NO: 180 - protein, 443 aa, synthetic construct
  REGION 1..443: note = Complete sequence of the heavy chain of 4H3C3
QVQLQQSGAELAKPGASVKMSCKASGYTFTSYWMHWVKQRPGQGLEWIGYINPSTGYAEYNQKFKDKATLTADKSSSTAYMQLSSLTSEDSAVYYCARLRFGSSGFAYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 181 - protein, 220 aa, synthetic construct
  REGION 1..220: note = Complete sequence of the light chain of 4H3C3
QIVLTQSPSSLAVSVGEKVTMSCKSSQSLLYSSNQKNYLAWYQQKPGQSPKLLIYWASTWESGVPDRFTGSGSGTDFTLTISSVKAEDLAVYYCQQYYSFPLTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 182 - DNA, 449 bp, synthetic construct
  misc_feature 1..449: note = Immature form of the recombinantly expressed VH sequence of 4H3C3
  source 1..449: mol_type = other DNA
gcggccgcaaactacaagacagacttgcaaaagaaggcatgcacagctcagcactgctctgttgcctggtcctcctgactggggtgagggcccaggtgcagctgcagcagtctggagctgaactggctaaacccggcgcttctgtgaaaatgtcttgtaaagcctcaggctataccttcacatcctactggatgcactgggtgaaacagagacccggccagggactggaatggattggatacatcaacccctcaaccggctacgccgagtataaccagaaattcaaggacaaggctaccctgaccgccgacaaaagcagctcaaccgcttacatgcagctgagcagcctgaccagcgaggattctgctgtgtactactgcgccagactgagattcggcagcagcggattcgcctactggggacagggaacactggtgacagtgtcttcc

SEQ ID NO: 183 - DNA, 357 bp, synthetic construct
  misc_feature 1..357: note = Mature form of VH sequence of 4H3C3
  source 1..357: mol_type = other DNA
caggtgcagctgcagcagtctggagctgaactggctaaacccggcgcttctgtgaaaatgtcttgtaaagcctcaggctataccttcacatcctactggatgcactgggtgaaacagagacccggccagggactggaatggattggatacatcaacccctcaaccggctacgccgagtataaccagaaattcaaggacaaggctaccctgaccgccgacaaaagcagctcaaccgcttacatgcagctgagcagcctgaccagcgaggattctgctgtgtactactgcgccagactgagattcggcagcagcggattcgcctactggggacagggaacactggtgacagtgtcttcc

SEQ ID NO: 184 - DNA, 431 bp, synthetic construct
  misc_feature 1..431: note = Immature form of the recombinantly expressed VL sequence of 4H3C3
  source 1..431: mol_type = other DNA
gcggccgcaaactacaagacagacttgcaaaagaaggcatgcacagctcagcactgctctgttgcctggtcctcctgactggggtgagggcccagatcgtgctgacccagtcaccaagcagcctggctgtgtctgtgggagaaaaggtgaccatgagctgcaaaagctcacagagcctgctgtacagctccaaccagaagaactacctggcctggtatcagcagaaacccggccagtcacccaaactgctgatctattgggccagcacctgggagagcggagtgcctgatagattcaccgggtccggaagcggaacagacttcacactgaccattagcagcgtgaaagctgaggacctggccgtgtactactgccagcagtactacagctttcccctgacctttggagccggcacaaagctggagatcaag

SEQ ID NO: 185 - DNA, 339 bp, synthetic construct
  misc_feature 1..339: note = Mature form of VL sequence of 4H3C3
  source 1..339: mol_type = other DNA
cagatcgtgctgacccagtcaccaagcagcctggctgtgtctgtgggagaaaaggtgaccatgagctgcaaaagctcacagagcctgctgtacagctccaaccagaagaactacctggcctggtatcagcagaaacccggccagtcacccaaactgctgatctattgggccagcacctgggagagcggagtgcctgatagattcaccgggtccggaagcggaacagacttcacactgaccattagcagcgtgaaagctgaggacctggccgtgtactactgccagcagtactacagctttcccctgacctttggagccggcacaaagctggagatcaag

SEQ ID NO: 186 - protein, 149 aa, synthetic construct
  REGION 1..149: note = Immature form of the recombinantly expressed VH sequence of 4H3C3, wherein positions 1-19 correspond to a leader sequence
GRKLQDRLAKEGMHSSALLCCLVLLTGVRAQVQLQQSGAELAKPGASVKMSCKASGYTFTSYWMHWVKQRPGQGLEWIGYINPSTGYAEYNQKFKDKATLTADKSSSTAYMQLSSLTSEDSAVYYCARLRFGSSGFAYWGQGTLVTVSS

SEQ ID NO: 187 - protein, 119 aa, synthetic construct
  REGION 1..119: note = Mature form of VH sequence of 4H3C3, without the leader sequence
QVQLQQSGAELAKPGASVKMSCKASGYTFTSYWMHWVKQRPGQGLEWIGYINPSTGYAEYNQKFKDKATLTADKSSSTAYMQLSSLTSEDSAVYYCARLRFGSSGFAYWGQGTLVTVSS

SEQ ID NO: 188 - protein, 143 aa, synthetic construct
  REGION 1..143: note = Immature form of the recombinantly expressed VL sequence of 4H3C3, wherein positions 1-19 correspond to a leader sequence
GRKLQDRLAKEGMHSSALLCCLVLLTGVRAQIVLTQSPSSLAVSVGEKVTMSCKSSQSLLYSSNQKNYLAWYQQKPGQSPKLLIYWASTWESGVPDRFTGSGSGTDFTLTISSVKAEDLAVYYCQQYYSFPLTFGAGTKLEIK

SEQ ID NO: 189 - protein, 113 aa, synthetic construct
  REGION 1..113: note = Mature form of VL sequence of 4H3C3, without the leader sequence
QIVLTQSPSSLAVSVGEKVTMSCKSSQSLLYSSNQKNYLAWYQQKPGQSPKLLIYWASTWESGVPDRFTGSGSGTDFTLTISSVKAEDLAVYYCQQYYSFPLTFGAGTKLEIK

SEQ ID NO: 190 - DNA, 15 bp, synthetic construct
  misc_feature 1..15: note = VH-CDR1 sequence of 4H3C3
  source 1..15: mol_type = other DNA
tcctactggatgcac

SEQ ID NO: 191 - DNA, 51 bp, synthetic construct
  misc_feature 1..51: note = VH-CDR2 sequence of 4H3C3
  source 1..51: mol_type = other DNA
tacatcaacccctcaaccggctacgccgagtataaccagaaattcaaggac

SEQ ID NO: 192 - DNA, 30 bp, synthetic construct
  misc_feature 1..30: note = VH-CDR3 sequence of 4H3C3
  source 1..30: mol_type = other DNA
ctgagattcggcagcagcggattcgcctac

SEQ ID NO: 193 - DNA, 51 bp, synthetic construct
  misc_feature 1..51: note = VL-CDR1 sequence of 4H3C3
  source 1..51: mol_type = other DNA
aaaagctcacagagcctgctgtacagctccaaccagaagaactacctggcc

SEQ ID NO: 194 - DNA, 21 bp, synthetic construct
  misc_feature 1..21: note = VL-CDR2 sequence of 4H3C3
  source 1..21: mol_type = other DNA
tgggccagcacctgggagagc

SEQ ID NO: 195 - DNA, 27 bp, synthetic construct
  misc_feature 1..27: note = VL-CDR3 sequence of 4H3C3
  source 1..27: mol_type = other DNA
cagcagtactacagctttcccctgacc

SEQ ID NO: 196 - protein, 5 aa, synthetic construct
  REGION 1..5: note = VH-CDR1 sequence of 4H3C3
SYWMH

SEQ ID NO: 197 - protein, 17 aa, synthetic construct
  REGION 1..17: note = VH-CDR2 sequence of 4H3C3
YINPSTGYAEYNQKFKD

SEQ ID NO: 198 - protein, 10 aa, synthetic construct
  REGION 1..10: note = VH-CDR3 sequence of 4H3C3
LRFGSSGFAY

SEQ ID NO: 199 - protein, 17 aa, synthetic construct
  REGION 1..17: note = VL-CDR1 sequence of 4H3C3, 7B1F3 and 2F5B11
KSSQSLLYSSNQKNYLA

SEQ ID NO: 200 - protein, 7 aa, synthetic construct
  REGION 1..7: note = VL-CDR2 sequence of 4H3C3
WASTWES

SEQ ID NO: 201 - protein, 9 aa, synthetic construct
  REGION 1..9: note = VL-CDR3 sequence of 4H3C3
QQYYSFPLT

SEQ ID NO: 202 - DNA, 1428 bp, synthetic construct
  misc_feature 1..1428: note = Complete sequence of the heavy chain of 7B1F3
  source 1..1428: mol_type = other DNA
gcggccgcaaactacaagacagacttgcaaaagaaggcatgcacagctcagcactgctctgttgcctggtcctcctgactggggtgagggccgaagtgcagctgcagcagagtggagctgagctggctaagcctggagctagtgtgaagatgagctgcaaggccagcggatacacatttaccaactactggatgcactgggtgaagcagagacccggacagggactggaatggattggatacgtgaaccccagaacaggatacaccgagtacaaccagaagttcaaagacaaagccaccctgaccgccgacaaaagcagcagaaccgcttacatgcagctgtccagcctgaccagcgaggattctgctgtgtacagttgcgccagaatcctgaacggcaccggatttgcctactggggacagggaaccctggtgacagtgtctgctgctaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccgcaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatgattctaga

SEQ ID NO: 203 - DNA, 762 bp, synthetic construct
  misc_feature 1..762: note = Complete sequence of the light chain of 7B1F3
  source 1..762: mol_type = other DNA
gcggccgcaaactacaagacagacttgcaaaagaaggcatgcacagctcagcactgctctgttgcctggtcctcctgactggggtgagggccgatattgtgctgacccagagccccagcagcctgacagtgtctgtgggagaaaagtttaccatgagctgtaagagcagccagagcctgctgtacagcagcaaccagaagaactacctggcctggtatcagcagaagcccggacagtcacccaagctgctgatttactgggccagcacccgggaaagcggagtgcctgatagattcaccggcagcggaagcggaaccgatttcacactggctatcagcagcgtgaagcccgaggatctggctatctactactgccagcagtactacagcttcccactgaccttcggcgccggaacaaagctggaaatcaagagagcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgattctaga

SEQ ID NO: 204 - protein, 472 aa, synthetic construct
  REGION 1..472: note = Complete sequence of the heavy chain of 7B1F3
GRKLQDRLAKEGMHSSALLCCLVLLTGVRAEVQLQQSGAELAKPGASVKMSCKASGYTFTNYWMHWVKQRPGQGLEWIGYVNPRTGYTEYNQKFKDKATLTADKSSRTAYMQLSSLTSEDSAVYSCARILNGTGFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 205 - protein, 250 aa, synthetic construct
  REGION 1..250: note = Complete sequence of the light chain of 7B1F3
GRKLQDRLAKEGMHSSALLCCLVLLTGVRADIVLTQSPSSLTVSVGEKFTMSCKSSQSLLYSSNQKNYLAWYQQKPGQSPKLLIYWASTRESGVPDRFTGSGSGTDFTLAISSVKPEDLAIYYCQQYYSFPLTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 206 - DNA, 446 bp, synthetic construct
  misc_feature 1..446: note = Immature form of the recombinantly expressed VH sequence of 7B1F3
  source 1..446: mol_type = other DNA
gcggccgcaaactacaagacagacttgcaaaagaaggcatgcacagctcagcactgctctgttgcctggtcctcctgactggggtgagggccgaagtgcagctgcagcagagtggagctgagctggctaagcctggagctagtgtgaagatgagctgcaaggccagcggatacacatttaccaactactggatgcactgggtgaagcagagacccggacagggactggaatggattggatacgtgaaccccagaacaggatacaccgagtacaaccagaagttcaaagacaaagccaccctgaccgccgacaaaagcagcagaaccgcttacatgcagctgtccagcctgaccagcgaggattctgctgtgtacagttgcgccagaatcctgaacggcaccggatttgcctactggggacagggaaccctggtgacagtgtctgct

SEQ ID NO: 207 - DNA, 354 bp, synthetic construct
  misc_feature 1..354: note = Mature form of VH sequence of 7B1F3
  source 1..354: mol_type = other DNA
gaagtgcagctgcagcagagtggagctgagctggctaagcctggagctagtgtgaagatgagctgcaaggccagcggatacacatttaccaactactggatgcactgggtgaagcagagacccggacagggactggaatggattggatacgtgaaccccagaacaggatacaccgagtacaaccagaagttcaaagacaaagccaccctgaccgccgacaaaagcagcagaaccgcttacatgcagctgtccagcctgaccagcgaggattctgctgtgtacagttgcgccagaatcctgaacggcaccggatttgcctactggggacagggaaccctggtgacagtgtctgct

SEQ ID NO: 208 - DNA, 431 bp, synthetic construct
  misc_feature 1..431: note = Immature form of the recombinantly expressed VL sequence of 7B1F3
  source 1..431: mol_type = other DNA
gcggccgcaaactacaagacagacttgcaaaagaaggcatgcacagctcagcactgctctgttgcctggtcctcctgactggggtgagggccgatattgtgctgacccagagccccagcagcctgacagtgtctgtgggagaaaagtttaccatgagctgtaagagcagccagagcctgctgtacagcagcaaccagaagaactacctggcctggtatcagcagaagcccggacagtcacccaagctgctgatttactgggccagcacccgggaaagcggagtgcctgatagattcaccggcagcggaagcggaaccgatttcacactggctatcagcagcgtgaagcccgaggatctggctatctactactgccagcagtactacagcttcccactgaccttcggcgccggaacaaagctggaaatcaag

SEQ ID NO: 209 - DNA, 339 bp, synthetic construct
  misc_feature 1..339: note = Mature form of VL sequence of 7B1F3
  source 1..339: mol_type = other DNA
gatattgtgctgacccagagccccagcagcctgacagtgtctgtgggagaaaagtttaccatgagctgtaagagcagccagagcctgctgtacagcagcaaccagaagaactacctggcctggtatcagcagaagcccggacagtcacccaagctgctgatttactgggccagcacccgggaaagcggagtgcctgatagattcaccggcagcggaagcggaaccgatttcacactggctatcagcagcgtgaagcccgaggatctggctatctactactgccagcagtactacagcttcccactgaccttcggcgccggaacaaagctggaaatcaag

SEQ ID NO: 210 - protein, 148 aa, synthetic construct
  REGION 1..148: note = Immature form of the recombinantly expressed VH sequence of 7B1F3, wherein positions 1-19 correspond to a leader sequence
GRKLQDRLAKEGMHSSALLCCLVLLTGVRAEVQLQQSGAELAKPGASVKMSCKASGYTFTNYWMHWVKQRPGQGLEWIGYVNPRTGYTEYNQKFKDKATLTADKSSRTAYMQLSSLTSEDSAVYSCARILNGTGFAYWGQGTLVTVSA

SEQ ID NO: 211 - protein, 118 aa, synthetic construct
  REGION 1..118: note = Mature form of VH sequence of 7B1F3, without the leader sequence
EVQLQQSGAELAKPGASVKMSCKASGYTFTNYWMHWVKQRPGQGLEWIGYVNPRTGYTEYNQKFKDKATLTADKSSRTAYMQLSSLTSEDSAVYSCARILNGTGFAYWGQGTLVTVSA

SEQ ID NO: 212 - protein, 143 aa, synthetic construct
  REGION 1..143: note = Immature form of the recombinantly expressed VL sequence of 7B1F3, wherein positions 1-19 correspond to a leader sequence
GRKLQDRLAKEGMHSSALLCCLVLLTGVRADIVLTQSPSSLTVSVGEKFTMSCKSSQSLLYSSNQKNYLAWYQQKPGQSPKLLIYWASTRESGVPDRFTGSGSGTDFTLAISSVKPEDLAIYYCQQYYSFPLTFGAGTKLEIK

SEQ ID NO: 213 - protein, 113 aa, synthetic construct
  REGION 1..113: note = Mature form of VL sequence of 7B1F3, without the leader sequence
DIVLTQSPSSLTVSVGEKFTMSCKSSQSLLYSSNQKNYLAWYQQKPGQSPKLLIYWASTRESGVPDRFTGSGSGTDFTLAISSVKPEDLAIYYCQQYYSFPLTFGAGTKLEIK

SEQ ID NO: 214 - DNA, 15 bp, synthetic construct
  misc_feature 1..15: note = VH-CDR1 sequence of 7B1F3
  source 1..15: mol_type = other DNA
aactactggatgcac

SEQ ID NO: 215 - DNA, 51 bp, synthetic construct
  misc_feature 1..51: note = VH-CDR2 sequence of 7B1F3
  source 1..51: mol_type = other DNA
tacgtgaaccccagaacaggatacaccgagtacaaccagaagttcaaagac

SEQ ID NO: 216 - DNA, 27 bp, synthetic construct
  misc_feature 1..27: note = VH-CDR3 sequence of 7B1F3
  source 1..27: mol_type = other DNA
atcctgaacggcaccggatttgcctac

SEQ ID NO: 217 - DNA, 21 bp, synthetic construct
  misc_feature 1..21: note = VL-CDR2 sequence of 7B1F3
  source 1..21: mol_type = other DNA
tgggccagcacccgggaaagc

SEQ ID NO: 218 - DNA, 27 bp, synthetic construct
  misc_feature 1..27: note = VL-CDR3 sequence of 7B1F3
  source 1..27: mol_type = other DNA
cagcagtactacagcttcccactgacc

SEQ ID NO: 219 - protein, 5 aa, synthetic construct
  REGION 1..5: note = VH-CDR1 sequence of 7B1F3
NYWMH

SEQ ID NO: 220 - protein, 17 aa, synthetic construct
  REGION 1..17: note = VH-CDR2 sequence of 7B1F3
YVNPRTGYTEYNQKFKD

SEQ ID NO: 221 - protein, 9 aa, synthetic construct
  REGION 1..9: note = VH-CDR3 sequence of 7B1F3
ILNGTGFAY

SEQ ID NO: 222 - protein, 7 aa, synthetic construct
  REGION 1..7: note = VL-CDR2 sequence of 7B1F3
WASTRES

SEQ ID NO: 223 - protein, 9 aa, synthetic construct
  REGION 1..9: note = VL-CDR3 sequence of 7B1F3
QQYYSFPLT

SEQ ID NO: 224 - DNA, 1431 bp, synthetic construct
  misc_feature 1..1431: note = Complete sequence of the heavy chain of 2F5B11
  source 1..1431: mol_type = other DNA
gcggccgcaaactacaagacagacttgcaaaagaaggcatgcacagctcagcactgctctgttgcctggtcctcctgactggggtgagggccgaagtgcagctgcaggaaagcggagctgagctggctaagcctggagcttctgtgaagatgagctgtaaggccagcggatacacatttagtagctattggattcattggatcaagcagagacctggccagggactggaatggattggatacatcaaccccaacaccgcctataccgagttcaaccagaaattcatggacaaggccaccctgaccgccgataaaagcagctcaaccgcctacatgcagctgaccagcctgacaagcgaagacagcgctgtgtactattgcgccaggctgagaagcgacagaaccggattcgcctactggggacagggaacactggtgacagtgagtgccgctaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccgcaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatgattctaga

SEQ ID NO: 225 - DNA, 762 bp, synthetic construct
  misc_feature 1..762: note = Complete sequence of the light chain of 2F5B11
  source 1..762: mol_type = other DNA
gcggccgcaaactacaagacagacttgcaaaagaaggcatgcacagctcagcactgctctgttgcctggtcctcctgactggggtgagggcccagatcgtgctgacccagtcaccaagcagcctggctgtgtctgtgggagaaaaggtgaccatgagctgcaaaagctcacagagcctgctgtacagctccaaccagaagaactacctggcctggtatcagcagaaacccggccagtcacccaaagtgctgatctactgggccagcaccagagaatacggcgtgcctgatagattcaccggcagcggaagcggaaccgatttcacactgactattagcagcgtgaaggctgaagacctggccgtgtactactgccagcagtactacagcttccccctgacctttggagccggaacaaagctggaaatcaagagagcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgattctaga

SEQ ID NO: 226 - protein, 473 aa, synthetic construct
  REGION 1..473: note = Complete sequence of the heavy chain of 2F5B11
GRKLQDRLAKEGMHSSALLCCLVLLTGVRAEVQLQESGAELAKPGASVKMSCKASGYTFSSYWIHWIKQRPGQGLEWIGYINPNTAYTEFNQKFMDKATLTADKSSSTAYMQLTSLTSEDSAVYYCARLRSDRTGFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 227 - protein, 250 aa, synthetic construct
  REGION 1..250: note = Complete sequence of the light chain of 2F5B11
GRKLQDRLAKEGMHSSALLCCLVLLTGVRAQIVLTQSPSSLAVSVGEKVTMSCKSSQSLLYSSNQKNYLAWYQQKPGQSPKVLIYWASTREYGVPDRFTGSGSGTDFTLTISSVKAEDLAVYYCQQYYSFPLTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 228 - DNA, 449 bp, synthetic construct
  misc_feature 1..449: note = Immature form of the recombinantly expressed VH sequence of 2F5B11
  source 1..449: mol_type = other DNA
gcggccgcaaactacaagacagacttgcaaaagaaggcatgcacagctcagcactgctctgttgcctggtcctcctgactggggtgagggccgaagtgcagctgcaggaaagcggagctgagctggctaagcctggagcttctgtgaagatgagctgtaaggccagcggatacacatttagtagctattggattcattggatcaagcagagacctggccagggactggaatggattggatacatcaaccccaacaccgcctataccgagttcaaccagaaattcatggacaaggccaccctgaccgccgataaaagcagctcaaccgcctacatgcagctgaccagcctgacaagcgaagacagcgctgtgtactattgcgccaggctgagaagcgacagaaccggattcgcctactggggacagggaacactggtgacagtgagtgcc

SEQ ID NO: 229 - DNA, 357 bp, synthetic construct
  misc_feature 1..357: note = Mature form of VH sequence of 2F5B11
  source 1..357: mol_type = other DNA
gaagtgcagctgcaggaaagcggagctgagctggctaagcctggagcttctgtgaagatgagctgtaaggccagcggatacacatttagtagctattggattcattggatcaagcagagacctggccagggactggaatggattggatacatcaaccccaacaccgcctataccgagttcaaccagaaattcatggacaaggccaccctgaccgccgataaaagcagctcaaccgcctacatgcagctgaccagcctgacaagcgaagacagcgctgtgtactattgcgccaggctgagaagcgacagaaccggattcgcctactggggacagggaacactggtgacagtgagtgcc

SEQ ID NO: 230 - DNA, 431 bp, synthetic construct
  misc_feature 1..431: note = Immature form of the recombinantly expressed VL sequence of 2F5B11
  source 1..431: mol_type = other DNA
gcggccgcaaactacaagacagacttgcaaaagaaggcatgcacagctcagcactgctctgttgcctggtcctcctgactggggtgagggcccagatcgtgctgacccagtcaccaagcagcctggctgtgtctgtgggagaaaaggtgaccatgagctgcaaaagctcacagagcctgctgtacagctccaaccagaagaactacctggcctggtatcagcagaaacccggccagtcacccaaagtgctgatctactgggccagcaccagagaatacggcgtgcctgatagattcaccggcagcggaagcggaaccgatttcacactgactattagcagcgtgaaggctgaagacctggccgtgtactactgccagcagtactacagcttccccctgacctttggagccggaacaaagctggaaatcaag

SEQ ID NO: 231 - DNA, 339 bp, synthetic construct
  misc_feature 1..339: note = Mature form of VL sequence of 2F5B11
  source 1..339: mol_type = other DNA
cagatcgtgctgacccagtcaccaagcagcctggctgtgtctgtgggagaaaaggtgaccatgagctgcaaaagctcacagagcctgctgtacagctccaaccagaagaactacctggcctggtatcagcagaaacccggccagtcacccaaagtgctgatctactgggccagcaccagagaatacggcgtgcctgatagattcaccggcagcggaagcggaaccgatttcacactgactattagcagcgtgaaggctgaagacctggccgtgtactactgccagcagtactacagcttccccctgacctttggagccggaacaaagctggaaatcaag

SEQ ID NO: 232 - protein, 149 aa, synthetic construct
  REGION 1..149: note = Immature form of the recombinantly expressed VH sequence of 2F5B11, wherein positions 1-19 correspond to a leader sequence
GRKLQDRLAKEGMHSSALLCCLVLLTGVRAEVQLQESGAELAKPGASVKMSCKASGYTFSSYWIHWIKQRPGQGLEWIGYINPNTAYTEFNQKFMDKATLTADKSSSTAYMQLTSLTSEDSAVYYCARLRSDRTGFAYWGQGTLVTVSA

SEQ ID NO: 233 - protein, 119 aa, synthetic construct
  REGION 1..119: note = Mature form of VH sequence of 2F5B11, without the leader sequence
EVQLQESGAELAKPGASVKMSCKASGYTFSSYWIHWIKQRPGQGLEWIGYINPNTAYTEFNQKFMDKATLTADKSSSTAYMQLTSLTSEDSAVYYCARLRSDRTGFAYWGQGTLVTVSA

SEQ ID NO: 234 - protein, 143 aa, synthetic construct
  REGION 1..143: note = Immature form of the recombinantly expressed VL sequence of 2F5B11, wherein positions 1-19 correspond to a leader sequence
GRKLQDRLAKEGMHSSALLCCLVLLTGVRAQIVLTQSPSSLAVSVGEKVTMSCKSSQSLLYSSNQKNYLAWYQQKPGQSPKVLIYWASTREYGVPDRFTGSGSGTDFTLTISSVKAEDLAVYYCQQYYSFPLTFGAGTKLEIK

SEQ ID NO: 235 - protein, 113 aa, synthetic construct
  REGION 1..113: note = Mature form of VL sequence of 4H3C3, without the leader sequence
QIVLTQSPSSLAVSVGEKVTMSCKSSQSLLYSSNQKNYLAWYQQKPGQSPKVLIYWASTREYGVPDRFTGSGSGTDFTLTISSVKAEDLAVYYCQQYYSFPLTFGAGTKLEIK

SEQ ID NO: 236 - DNA, 15 bp, synthetic construct
  misc_feature 1..15: note = VH-CDR1 sequence of 2F5B11
  source 1..15: mol_type = other DNA
agctattggattcat

SEQ ID NO: 237 - DNA, 51 bp, synthetic construct
  misc_feature 1..51: note = VH-CDR2 sequence of 2F5B11
  source 1..51: mol_type = other DNA
tacatcaaccccaacaccgcctataccgagttcaaccagaaattcatggac

SEQ ID NO: 238 - DNA, 30 bp, synthetic construct
  misc_feature 1..30: note = VH-CDR3 sequence of 2F5B11
  source 1..30: mol_type = other DNA
ctgagaagcgacagaaccggattcgcctac

SEQ ID NO: 239 - DNA, 51 bp, synthetic construct; identical to SEQ ID NO: 193
  misc_feature 1..51: note = VL-CDR1 sequence of 2F5B11
  source 1..51: mol_type = other DNA

SEQ ID NO: 240 - DNA, 21 bp, synthetic construct
  misc_feature 1..21: note = VL-CDR2 sequence of 2F5B11
  source 1..21: mol_type = other DNA
tgggccagcaccagagaatac

SEQ ID NO: 241 - DNA, 27 bp, synthetic construct
  misc_feature 1..27: note = VL-CDR3 sequence of 2F5B11
  source 1..27: mol_type = other DNA
cagcagtactacagcttccccctgacc

SEQ ID NO: 242 - protein, 5 aa, synthetic construct
  REGION 1..5: note = VH-CDR1 sequence of 2F5B11
SYWIH

SEQ ID NO: 243 - protein, 17 aa, synthetic construct
  REGION 1..17: note = VH-CDR2 sequence of 2F5B11
YINPNTAYTEFNQKFMD

SEQ ID NO: 244 - protein, 10 aa, synthetic construct
  REGION 1..10: note = VH-CDR3 sequence of 2F5B11
LRSDRTGFAY

SEQ ID NO: 245 - protein, 7 aa, synthetic construct
  REGION 1..7: note = VL-CDR2 sequence of 2F5B11
WASTREY

SEQ ID NO: 246 - protein, 9 aa, synthetic construct
  REGION 1..9: note = VL-CDR3 sequence of 2F5B11
QQYYSFPLT

SEQ ID NO: 247 - skipped

SEQ ID NO: 248 - protein, 17 aa, synthetic construct
  REGION 1..17: note = X2 = I or V; X5 = S, R or N; X7 = G or A; X9 = A or T; X11 = Y or F; X16 = K or M
  VARIANT 2: note = I or V
  VARIANT 5: note = S, R or N
  VARIANT 7: note = G or A
  VARIANT 9: note = A or T
  VARIANT 11: note = Y or F
  VARIANT 16: note = K or M
YXNPXTXYXEXNQKFXD

SEQ ID NO: 249 - protein, 10 aa, synthetic construct
  REGION 1..10: note = X1 = L or I; X2 = R or L; X3 = F, N or S; X4 = G or D; X5 = S, R or blank; X6 = S or T
  VARIANT 1: note = L or I
  VARIANT 2: note = R or L
  VARIANT 3: note = F, N or S
  VARIANT 4: note = G or D
  VARIANT 5: note = S, R or blank
  VARIANT 6: note = S or T
XXXXXXGFAY

SEQ ID NO: 250 - protein, 7 aa, synthetic construct
  REGION 1..7: note = X5 = W or R; X7 = S or Y
  VARIANT 5: note = W or R
  VARIANT 7: note = S or Y
WASTXEX

SEQ ID NO: 251 - DNA, 51 bp, synthetic construct
  misc_feature 1..51: note = VL-CDR1 sequence of 7B1F3
  source 1..51: mol_type = other DNA
aagagcagccagagcctgctgtacagcagcaaccagaagaactacctggcc

SEQ ID NO: 252 - DNA, 92 bp, synthetic construct
  misc_feature 1..92: note = Nucleic acid sequence encoding the leader sequence of recombinantly expressed VH sequence of ECD1-binding molecules
  source 1..92: mol_type = other DNA
gcggccgcaaactacaagacagacttgcaaaagaaggcatgcacagctcagcactgctctgttgcctggtcctcctgactggggtgagggcc

SEQ ID NO: 253 - protein, 30 aa, synthetic construct
  REGION 1..30: note = Leader sequence of recombinantly expressed VH sequence of ECD1-binding molecules
GRKLQDRLAKEGMHSSALLCCLVLLTGVRA

SEQ ID NO: 254 - protein, 367 aa, synthetic construct
  REGION 1..367: note = US28 fusion protein, comprising US28 sequence from HCMV TB40/E strain
MTPTTTTTELTTEFEYDLGATPCTFTDVLNQSKPVTLFLYGVVFLFGSVGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNVELMLGAFVIPLSVISYCYYRISRIVAVSQSRHKGRIVRVLIAVVLVFIIFWLPYHLTLFVDTLKLLKWISSSCEFERSLKRALILTESLAFCHCCLNPLLYVFVGTKFRQELHCLLAEFRQRLFSRDVSWYHSMSFSRRSSPSRRETSSDTLSDEVCRVSQIIPAAALEGSHHHHHH

SEQ ID NO: 255 - protein, 366 aa, synthetic construct
  REGION 1..366: note = US28 fusion protein, comprising US28 sequence from HCMV mutated strain
MTQTTTTELTTEFDYDLGAALCTLTDVLNQSKPITLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNVELMLGAFVIPLSVISYCYYRISRIVAVSQSRHKGRIVRVLIAVVLVFIIFWLPYHLTLFLDTLKLLKWISSSCESEKSLKRALILTESLAFCHCCLNPLLYVFVGTKFRQELHCLLAEFRQRLFSRDVSWYHSMSFSRRSSPSRRETSSDTLSDEVCRVSQIIPAAALEGSHHHHHH

SEQ ID NO: 256 - protein, 119 aa, synthetic construct
QVQLVQSGAEVKKPGASVKVSCKASGYTFTSYWMHWVRQAPGQGLEWIGYINPSTGYAEYNQKFQGRATLTADKSSSTAYMELSSLRSEDTAVYYCARLRFGSSGFAYWGQGTLVTVSS

SEQ ID NO: 257 - protein, 119 aa, synthetic construct
QVQLVQSGAEVKKPGASVKVSCKASGYTFTSYWMHWVRQAPGQGLEWMGYINPSTGYAEYNQKFQGRVTMTADKSTSTAYMELSSLRSEDTAVYYCARLRFGSSGFAYWGQGTLVTVSS

SEQ ID NO: 258 - protein, 119 aa, synthetic construct
QVQLVQSGAEVKKPGASVKVSCKASGYTFTSYWMHWVRQAPGQGLEWIGYINPSTGYAEYNQKLQGRATLTADKSSSTAYMELRSLRSDDTAVYYCARLRFGSSGFAYWGQGTLVTVSS

SEQ ID NO: 259 - protein, 119 aa, synthetic construct
QVQLVQSGAEVKKPGASVKVSCKASGYTFTSYWMHWVRQAPGQGLEWMGYINPSTGYAEYNQKLQGRVTMTADKSTSTAYMELRSLRSDDTAVYYCARLRFGSSGFAYWGQGTLVTVSS

SEQ ID NO: 260 - protein, 113 aa, synthetic construct
QIVLTQSPDSLAVSLGERATINCKSSQSLLYSSNQKNYLAWYQQKPGQPPKLLIYWASTWESGVPDRFSGSGSGTDFTLTISSLQAEDVAVYYCQQYYSFPLTFGGGTKLEIK

SEQ ID NO: 261 - protein, 113 aa, synthetic construct
DIVLTQSPDSLAVSLGERATINCKSSQSLLYSSNQKNYLAWYQQKPGQPPKLLIYWASTRESGVPDRFSGSGSGTDFTLTISSLQAEDVAVYYCQQYYSFPLTFGGGTKLEIK

SEQ ID NO: 262 - protein, 113 aa, synthetic construct
QIVLTQSPLSLPVTPGEPASISCRSSQSLLYSSNQKNYLAWYLQKPGQSPQLLIYWASTWESGVPDRFSGSGSGTDFTLKISRVEAEDVGVYYCQQYYSFPLTFGGGTKLEIK

SEQ ID NO: 263 - protein, 113 aa, synthetic construct
DIVLTQSPLSLPVTPGEPASISCRSSQSLLYSSNQKNYLAWYLQKPGQSPQLLIYWASTRESGVPDRFSGSGSGTDFTLKISRVEAEDVGVYYCQQYYSFPLTFGGGTKLEIK

SEQ ID NO: 264 - protein, 118 aa, synthetic construct
QVQLVQSGAEVKKPGASVKVSCKASGYTFTNYWMHWVRQAPGQGLEWIGYVNPRTGYTEYNQKFQGRATLTADKSSRTAYMELSSLRSEDTAVYSCARILNGTGFAYWGQGTLVTVSS

SEQ ID NO: 265 - protein, 118 aa, synthetic construct
QVQLVQSGAEVKKPGASVKVSCKASGYTFTNYWMHWVRQAPGQGLEWMGYVNPRTGYTEYNQKFQGRVTMTADKSTSTAYMELSSLRSEDTAVYYCARILNGTGFAYWGQGTLVTVSS

SEQ ID NO: 266 - protein, 118 aa, synthetic construct
QVQLVQSGAEVKKPGASVKVSCKASGYTFTNYWMHWVRQAPGQGLEWIGYVNPRTGYTEYNQQLQGRATLTADKSSRTAYMELRSLRSDDTAVYSCARILNGTGFAYWGQGTLVTVSS

SEQ ID NO: 267 - protein, 118 aa, synthetic construct
QVQLVQSGAEVKKPGASVKVSCKASGYTFTNYWMHWVRQAPGQGLEWMGYVNPRTGYTEYNQQLQGRVTMTADKSTSTAYMELRSLRSDDTAVYYCARILNGTGFAYWGQGTLVTVSS

SEQ ID NO: 268 - protein, 113 aa, synthetic construct
DIVLTQSPDSLAVSLGERATINCKSSQSLLYSSNQKNYLAWYQQKPGQPPKLLIYWASTRESGVPDRFSGSGSGTDFTLTISSLQAEDVAIYYCQQYYSFPLTFGGGTKLEIK

SEQ ID NO: 269 - protein, 113 aa, synthetic construct
DIVLTQSPLSLPVTPGEPASISCRSSQSLLYSSNQKNYLAWYLQKPGQSPQLLIYWASTRESGVPDRFSGSGSGTDFTLKISRVEAEDVGIYYCQQYYSFPLTFGGGTKLEIK

SEQ ID NO: 270 - protein, 118 aa, synthetic construct
QVQLVQSGAEVKKPGASVKVSCKASGYTFTNYWMHWVRQAPGQGLEWIGYVNPRTGYTEYNQKFQGRATLTADKSSRTAYMELSSLRSEDTAVYSCARILQGTGFAYWGQGTLVTVSS

SEQ ID NO: 271 - protein, 118 aa, synthetic construct
QVQLVQSGAEVKKPGASVKVSCKASGYTFTNYWMHWVRQAPGQGLEWMGYVNPRTGYTEYNQKFQGRVTMTADKSTSTAYMELSSLRSEDTAVYYCARILQGTGFAYWGQGTLVTVSS

SEQ ID NO: 272 - protein, 367 aa, synthetic construct
MTPTTTTAELTTEFDYDDEATPCVLTDVLNQSKPVTLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNVELMLGAFVIPLSVISYCYYRISRIVAVSQSRHKGRIVRVLIAVVLVFIIFWLPYHLTLFVDTLKLLKWISSSCEFEKSLKRALILTESLAFCHCCLNPLLYVFVGTKFRQELHCLLAEFRQRLFSRDVSWYHSMSFSRRSSPSRRETSSDTLSDEACRVSQIIPAAALEGSHHHHHH

SEQ ID NO: 273 - protein, 367 aa, synthetic construct
MTPTTTTAELTTEFDYDEDATPCVFTDVLNQSKPVTLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNVELMLGAFVIPLSVISYCYYRISRIVAVSQSRHKGRIVRVLIAVVLVFIIFWLPYHLTLFVDTLKLLKWISSSCEFERSLKRALILTESLAFCHCCLNPLLYVFVGTKFRQELHCLLAEFRQRLFSRDVSWYHSMSFSRRGSPSRRETSSDTLSDEVCRVSQIIPAAALEGSHHHHHH

SEQ ID NO: 274-292 - skipped (19 entries)

SEQ ID NO: 293 - protein, 17 aa, synthetic construct
YINPSTGYAEYNQKFQG

SEQ ID NO: 294 - protein, 17 aa, synthetic construct
YINPSTGYAEYNQKLQG

SEQ ID NO: 295 - protein, 17 aa, synthetic construct
RSSQSLLYSSNQKNYLA

SEQ ID NO: 296 - protein, 17 aa, synthetic construct
YVNPRTGYTEYNQKFQG

SEQ ID NO: 297 - protein, 17 aa, synthetic construct
YVNPRTGYTEYNQQLQG

SEQ ID NO: 298 - protein, 9 aa, synthetic construct
ILQGTGFAY

SEQ ID NO: 299 - protein, 17 aa, synthetic construct
  VARIANT 2: note = I or V
  VARIANT 5: note = S, R or N
  VARIANT 7: note = G or A
  VARIANT 9: note = A or T
  VARIANT 11: note = Y or F
  VARIANT 14: note = K or Q
  VARIANT 15: note = F or L
  VARIANT 16: note = K, M or Q
  VARIANT 17: note = D or G
YXNPXTXYXEXNQXXXX

SEQ ID NO: 300 - protein, 10 aa, synthetic construct
  VARIANT 1: note = L or I
  VARIANT 2: note = R or L
  VARIANT 3: note = F, N, S or Q
  VARIANT 4: note = G or D
  VARIANT 5: note = S, R or blank
  VARIANT 6: note = S or T
XXXXXXGFAY

SEQ ID NO: 301 - protein, 17 aa, synthetic construct
  VARIANT 1: note = K or R
XSSQSLLYSSNQKNYLA

(19) United States
(12) Patent Application Publication
(10) Pub. No.: US 2025/0296984 A1
(43) Pub. Date: Sep. 25, 2025

(54) THERAPEUTIC AND DIAGNOSTIC AGENTS AND USES THEREOF

(71) Applicant: THELPER AS, Oslo (NO)

(72) Inventor: Katja Vetvik, Oslo (NO)

(21) Appl. No.: 18/832,769

(22) PCT Filed: Jan. 30, 2023
(86) PCT No.: PCT/EP2023/052199

§ 371 Date: Jul. 24, 2024

(30) Foreign Application Priority Data
Jan. 28, 2022 (GB) 2201137.3

(51) Int. Cl. C07K 16/08; A61K 39/00; A61K 39/245; G01N 33/569

(52) U.S. Cl. CPC C07K 16/089; A61K 39/245; G01N 33/56994; A61K 2039/505; C07K 2317/24; C07K 2317/31; C07K 2317/41; C07K 2317/565; C07K 2317/92; G01N 2333/03; G01N 2500/04

(57) ABSTRACT

The present invention provides binding molecules having one or more of (preferably all of) highly specific binding to the US28 protein of human cytomegalovirus (HCMV), very low levels of non-specific binding to healthy (non-infected) cells, and/or a strain-agnostic binding ability, as well as nucleic acid molecules encoding the said binding molecules. The binding molecules are designed to bind to a newly-identified epitopic region within extracellular domain 1 (ECD1) of a US28 protein of human cytomegalovirus (HCMV), the first of the four extracellular domains presented by US28, corresponding to positions 1 to 37 of the US28 protein sequence as defined by SEQ ID NO:5. The binding molecules of the present invention have been demonstrated to have excellent binding properties, including particular binding specificity for aggressive and/or metastasizing HCMV-infected cancers, including breast cancers. In certain preferred embodiments, the binding molecule is selected from an antibody (including, for example, a BiTE antibody) and a chimeric antigen receptor (CAR), or functional variants, fragments, fusion proteins, and/or conjugates thereof. Also provided are cells expressing said binding molecules, such as CAR-expressing cells, including CAR-T cells, CAR-NK cells, and CAR-M cells.

### CROSS REFERENCE TO RELATED APPLICATIONS

This application is a U.S. National Stage Application filed under 35 U.S.C. § 371, based on International Patent Application No. PCT/EP2023/052199, filed on Jan. 30, 2023, which claims priority to UK Patent Application No. GB2201137.3, filed on Jan. 28, 2022. The entire contents of each of the above applications are incorporated herein by reference.

### STATEMENT REGARDING SEQUENCE LISTING

The instant application contains a XML Sequence Listing which has been submitted electronically in .XML format and is hereby incorporated by reference in its entirety. Said XML Sequence Listing, created on Jul. 24, 2024, is named 127275-11201_Sequence_Listing.xml and is 394,926 bytes in size. The sequence listing contained in this .XML file is part of the specification and is hereby incorporated by reference herein in its entirety.

### FIELD OF INVENTION

The present invention relates to the field of virology. More specifically, the invention relates to therapeutic and diagnostic agents targeting a specific region of the US28 protein, as encoded by human cytomegalovirus (HCMV), and therapies related thereto including but not limited to HCMV-infected cancers and other conditions associated with latent or lytic HCMV infections.

### REFERENCES

The listing or discussion of an apparently prior-published document in this specification should not necessarily be taken as an acknowledgement that the document is part of the state of the art or is common general knowledge. The references disclosed, to the extent that they provide exemplary procedural or other details supplementary to those set forth herein, are specifically incorporated herein by reference.

### BACKGROUND

Human cytomegalovirus (HCMV), also known as human herpes Virus 5 (HHV-5), is a ubiquitous, opportunistic DNA virus carried by 56-94% of the population worldwide (Geisler et al, Cancers, 2019, 11: 1842; Zuhair et al, Rev Med Viro, 2019. 29(3): p. e2034).

In most immunocompetent individuals, HCMV infections are asymptomatic, remain undiagnosed and are considered harmless, since viral replication is well-controlled by the host immune system (Boeckh and Geballe, J Clin Invest, 2011. 121(5): p. 1.673-80).

Similar to all herpesviruses, after primary infection, HCMV establishes a life-long persistence as a latent infection. The latent infection is characterized by low-level or non-existent virus replication with the viral genome residing predominantly in the CD34⁺ hematopoietic progenitor cell population residing in the bone marrow (Collins-McMillen et al., Viruses, 2018. 10(8)).

It is assumed that latent HCMV may intermittently reactivate in a stochastic manner unless continuously controlled by the host immune system. For this reason, the virus may cause complications in certain circumstances, for example in immunocomnpromised patients, in whom not only primary HCMV infection, but also re-infection or reactivation can cause a life-threatening disease that affects many organs causing considerable morbidity and mortality (Boeckh and Geballe, supra; Griffiths et al, J Pathol, 2015. 235(2): p. 288-97).

HCMV is one of the most common congenital viral infections and most important cause of birth defects (Davis et al, Birth Defects Res, 2017. 109(5): p. 336-346). It is becoming increasingly clear that HCMV infection over the life-course may also play a role in the pathogenesis of atherosclerosis, autoimmune diseases, and several malignancies, particularly glioblastoma multiforme (Soderberg-Naucler, J Intern Med, 2006, 259(3): p. 219-46; Cobbs, Curr Opin Virol, 2019. 39: p. 49-59). HCMV serostatus may additionally impact the clinical course of burns, trauma, and sepsis (Soderberg-Naucler, 2006, supra; Limaye, et al, JAMA, 2008. 300(4): p. 413-22; Osawa & Singh, Crit Care, 2009. 13(3): p. R68).

Latent HCMV infection can be reactivated during an inflammatory process when the progenitor cells differentiate into monocyte/infiltrating macrophages or dendritic cells (DCs), and these cells can disseminate the virus to peripheral organs (Soderberg-Naucler et al, Cell, 1997, 91: 119-126). Reactivated HCMV, carried by these inflammatory cells, can reach all body tissues, and infect and replicate in a broad number of cell types (Ljungrnan et al., Infect. Dis. Clin. North. Am., 2010, 24: 319-337). The infection is further transmitted by all body fluids, including saliva and breast milk (Hamprecht et al., Lancet, 2001, 357: 513-518). Ninety percent of breast milk samples from HCMV seropositive women contain the virus, and that results in about 30% HCMV prevalence in children at one year of age. Nursing and parental contact, therefore, constitutes an important route to acquiring the HCMV infection in early infancy or childhood (Hamprecht et al, 2001, supra).

### The Cytomegalovirus Genome and Genetic Diversity:

As discussed in Berg et al, 2019, PLoS ONE 14(9): e0222053, the CMV genome consists of monopartite, linear, double-stranded DNA and is roughly 235 kb in size. It contains more than 750 translated ORFs (Stern-Ginossar et al., Science, 2012, 338(6110):1088-93) which can be divided into two regions—the unique long (UL) and unique short (US) regions—flanked by terminal and internal inverted repeats.

Cytomegalovirus has adapted a wide range of strategies to avoid immune detection and facilitate dissemination of infection. These strategies are based on manipulation and modulation of the host's immune response during infection, e.g. by expression of virally encoded homologs of receptors and ligands important for the normal function of the human immune system. By encoding a 2 to 3-fold greater number of gene products than other human herpesviruses, many of which have been shown to interact with and manipulate the human immune system (Mocarski, Trends Microbiol, 2002; 10(7):332-9), CMV has an unparalleled number of tools available for modifying the host's immune response.

The genetic variation between circulating CMV strains is large and a recent study reported that 75% of the strains contain disruptive mutations and polymorphisms in several genes (Sijmons et al., J. Virol., 2015, 89(15): 7673-7695). In order to exclude disruptive mutations due to serial passage, the authors of the study only used strains passaged 1-2 times and verified most of the observed mutations directly from clinical samples. For the genes UL40 and UL111A, mutations causing functional knockouts were found in 9.9% and 5.5% of the investigated strains, respectively (Sijmons et al., 2015, supra). UL111A is a functional interleukin-10 homolog that can inhibit a normal immune response (Mocarski, 2002, supra; Engel and Angulo, Adv Exp Med Biol, 2012, 738:256-76). The signal peptide of UL40 facilitates surface expression of HLA-E on infected cells, which is a ligand for a natural killer cell inhibitory receptor (Wilkinson et al, J. Clin Virol., 2008; 41(3):206-12).

Other CMV genes that are also highly variable are the chemnokine homolog UL146 where 14 distinct genotypes have been identified (Dolan et al., 7 Gen Virol, 2004, 85(Pt 5):1301-12), and the chemokine scavenging receptor (Kiedal et al., FEBS Lett., 1998; 441(2):209-14) and G protein-coupled receptor US28 where numerous N-terminal polymorphisms have been reported (Goffard et al., Virus Genes, 2006; 33(2):175-81; Arav-Boger et al, J Infect Dis., 2002; 186(8):1057-64).

Berg et al, 2019 (supra) reports that this degree of genetic diversity is not observed for other human herpesviruses (Sijmons et al, 2015, supra) and poses the question of why CMV exerts such variability among important immunomodulatory genes and how it affects the virus-host interaction.

It is considered that a large part of the HCMV pathogenesis is associated with viral latency, which is closely linked to virus ability to escape from the humoral and cellular host immune responses through a number of mechanisms (Manandhar et al., Int J Mol Sci, 2019. 20(15)). One of the most important of such mechanisms include this high, ever-changing genetic diversity of the HCMV. The HCMV genome varies between different individuals and even within the same host (Gorzer et al., J Virol, 2010, 84(14): 7195-7203; Renzette et al., PLoS Pathog, 2011, 7(5): e1001344; Renzette et al., Curr Opin Virol, 2014. 8: 109-15; Renzette et al., Proc Natl Acad Sci USA, 2015, 112(30): E4120-8; Renzette et al., J Virol, 2017, 91(5)). New host infections give rise to a unique viral strain for each infected individual and generate selection events where a new genotype becomes dominant due to the selective pressure of the immune response (Renzette et al., 2011, supra). It is possible that both viral and host factors can contribute to fostering viral genetic drift during the HCMV infection (Vabret et al., Trends Imrnunoi, 2017, 38(1): 53-65; Christensen & Paludan, Cell Mol Immunol, 2017, 14(1): 4-13). In addition, each patient is likely to be infected with multiple CMV strains as previously extensively reported in the literature (Renzette et al., 2015, supra). The presence of multiple strains in the same individual enable recombination from the different HCMV strains. Recombination is considered to stand out as a major driver of HCMV genetic diversity (Suarez et al., J Infect Dis, 2019, 220(5): 781-791; Sijrnons et al., 2015, supra; Lassalle et al., Virus Evol, 2016, 2(1): vew017; Cudini et al., Proc Natl Acad Sci USA, 2019, 116(12): 5693-5698). Reassorting the highly diverse regions would create new combinations to ensure efficient immune evasion, which also can impact the pathogenicity of the virus. Consistently, mixed HCMV infection has been associated with poor clinical outcome in immunocompromised individuals in several studies (Coaquette et al., Clin Infect Dis, 2004, 39(2): 155-161; Lisboa et al., Transpl Infect Dis, 2012, 14(2): 132-140; Houldcroft et al., Front Microbiol, 2016, 7: 1317).

HCMV diversity is moreover driven by genetic polymorphisms, which are not evenly distributed across the genome (Sijmons et al., 2015, supra). Selection is stronger in protein regions exposed on the virion surface and for viral proteins expressed at the host cell membrane in the extracellular domains (Mozzi et al., PLoS Pathog, 2020, 16(5): e1008476). The selective pressure exerted by the host immune system has likely played a major role in the shaping of genetic diversity among circulating HCMV strains. Thus, several sites targeted by positive selection are located within epitopes recognized by human antibodies or in protein regions that directly interact with host molecules involved in immune response (Sijmons et al., 2015, supra; Mozzi et al., 2020, supra). These features are consistent with an ongoing hide and seek interplay between HCMV and the human immune system.

The strain variability and constantly mutating virus make both viral diagnostics and the vaccine and drug development demanding against the HCMV and set limits for the current antiviral drug treatment. Vaccine development against HCMV has over many years been of high priority for the medical community, but no effective vaccines have so far been approved against HCMV.

### Oncogenic Properties of HCMV

HCMV encoded proteins display diverse oncogenic functions (Geisler et al, 2019, supra). Upon entry into the host cell, tegument proteins of the HCMV virion, such as pUL48, are released, disabling cellular intrinsic and innate immune responses, and promoting enhanced metabolic activity of the host cells (Kumari et al., Cell Death Dis., 2017, 8: e3078). These HCMV-encoded proteins may enable the cells to surpass the G1-phase to facilitate rapid cell division (Kumari et al., 2017, supra). Through upregulation of anti-apoptotic genes and downregulation of pro-apoptotic genes, cells enter a state of enhanced survival.

After the entry of viral DNA into the cell nucleus, cellular RNA polymerases I and II (Pol I and II) are employed to transcribe the viral genes by binding to the major immediate early promoter (MIEP) (Kostopoulou et al., Oncotarget, 2017, 8: 96536-96552). The first genes that are expressed are the immediate early (IE) genes. The IE proteins derived from such genes act as transcription factors controlling both early and late viral gene expression, and direct host gene expression. Such proteins are necessary to establish lytic infection and are crucial for viral reactivation from latency (Kumari et al., 2017, supra; Tamrakar et al., J. ViroL., 2005, 79: 15477-15493). Lytic HCMV infection leads to a dysregulated cell cycle, and the IE gene products interfere with key cellular factors, including retinoblastoma protein family (Rb), cyclins, p53, Wnt, phosphatidylinositol 3-kinase/Akt, human telomerase reverse transcriptase (hTERT), and NF-κB to increase the immortal properties of infected cells (Moussawi et al., Sci. Rep., 2018, 8: 12574). These pathways are commonly activated in cancer cells. Activation of mitogenic signals, delivered by proto-oncogenes such as Fos and Myc, can be induced by IE proteins in HCMV infected cells (Hagemeier et al., J. Virol., 1992, 66: 4452-4456). Moreover, the MYB gene is induced in HCMV infected cells resembling the enhanced MYB gene expression in HPV-related carcinoma (Moussawi et al., 2018, supra). In addition to the mitogenic signals, HCMV infection causes chromosomal aberrations through deterioration of DNA repair pathways, resulting in genetic instability in the infected cells (Straat et al., J. Natl. Cancer Inst., 2009, 101: 488-497; Siew et al., J. Biomed. Sci., 2009, 16: 107). This drives the development of genetic mutations.

Various HCMV-encoded, G-protein-coupled-receptor (GPCR)-like proteins, including US27, US28, UL33, and UL78, have been reported to display important oncogenic functions (Heukers et al., Oncogene, 2018, 37: 4110-4121). G-proteins activate both metabolic and oncogenic key signaling pathways, such as cAMP and the PI3K signaling pathways, of which the latter is critical for the emergence of anchorage-independent growth and oncogenic transformation of epithelial cells (Moussawi et al., 2018, supra; Boroughs et al., Nat. Cell Biol., 2015, 17: 351-359). The HCMV-2.7 early gene transcript is a long non-coding (Inc) RNA that interacts directly with complex I of the respiratory chain in mitochondria, preventing mitochondria-induced cell death by inhibiting Fas-ligand interactions and granzyme B by binding to caspase 8, improving the oxidative capacity and maintaining energy production in the infected cells (Reeves et al., Science, 2007, 316: 1345-1348).

HCMV has also developed several ways to manipulate the innate and adaptive immune responses to decrease its immune surveillance and improve its chances of surviving in its immunocompetent host, which may well account for the important immune evasive mechanisms in the HCMV-infected cancer cells. HCMV encodes multiple proteins that modulate NK cell recognition of the infected cells (Fielding et al., PLoS Pathog., 2014, 10: e1004058), and increase CD8⁺ T cell tolerability for the viral proteins. HCMV encoded proteins can stimulate the development of an immature phenotype of DC, which reduces the activation of CD4⁺ T cell responses (Wagner et al., J. Leukoc Biol., 2008, 83: 56-63), and additionally, decreases the elimination of infected cells by CD8⁺ cytotoxic T cells.

### Hcmv Therapeutic Targets:

Currently, the only antiviral therapy for HCMV available relies on nucleoside analogs, such as ganciclovir (GCV) and valganciclovir (VAL-GCV) (Rawlinson et al., Lancet Infect Dis, 2017, 17(6): e177-e188; James & Kimberlin, Curr Opin Pediatr, 2016, 28(1): 81-85), which have several disadvantages such as poor bioavailability, toxic side-effects and the risk of developing drug resistance. Current results indicate that DNA polymerase (UL54) and viral phosphotransferase (UL97), two highly polymorphic HCMV genes, play important role in drug resistance against GCV (Komatsu et al., Antiviral Res, 2014, 101: 12-25).

In addition, importantly, the existing antivirals can only be used to treat lytic HCMV infections, but cannot clear the latent virus. Eradication or reducing the latent reservoir would be a favorable way to reduce the burden of HCMV related diseases in several patient groups.

The previously developed anti-HCMV drugs, such as ganciclovir (GCV), foscarnet (FOS), and cidofovir (CDV), all target the UL54 viral DNA polymerase. Yet, antiviral toxicity and HCMV antiviral drug resistance constitute a growing therapeutic challenge in the transplant setting and so new anti-HCMV drugs with novel viral targets are highly needed (Burrel et al, 2014, Lack of influence of human cytomegalovirus (HCMV) susceptibility to current antiviral drugs on HCMV-encoded US28 chemokine 5 receptor polymorphism, Poster presentation at ECCMID 2014, Barcelona).

Burrel et al, 2014 (supra) taught that, because of its potential roles in viral dissemination and persistence as well as in smooth muscle cell migration and tumorigenesis, HCMV-encoded US28 constitutes a potential target for novel antiviral therapies.

HCMV US28 is a seven transmembrane protein belonging to a class of G-protein coupled receptors (GCPRs). GCPRs constitute the largest family of proteins targeted by approved drugs (Sriram & Insel, Mol Pharmacol, 2018. 93(4): 251-258), and share common architecture, each consisting of a single polypeptide with an extracellular N-terminus, an intracellular C-terminus and seven hydrophobic transmembrane domains (TM1-TM7) linked by three extracellular loops (ECL1-3) (Alexander et al., Br J Pharmacol, 2019, 176 Suppl 1: S21-S141).

The full sequence of US28 as encoded by HCMV strain DB (Accession number KT959235) is provided in the present application as SEQ ID NO: 5, wherein:
the N-terminal extracellular domain (also referred to herein as ECD1 as it is the first extracellular domain) is provided herein as SEQ ID NO: 1 and corresponds to positions 1-37 of SEQ ID NO:5,
the first extracellular loop (ECL1; although also referred to herein as ECD2 as it is the second extracellular domain) is provided herein as SEQ ID NO: 2 and corresponds to positions 91-101 of SEQ ID NO:5,
the second extracellular loop (ECL2; although also referred to herein as ECD3 as it is the third extracellular domain) is provided herein as SEQ ID NO: 3 and corresponds to positions 167-183 of SEQ ID NO:5; and
the third extracellular loop (ECL3; although also referred to herein as ECD4 as it is the fourth extracellular domain) is provided herein as SEQ ID NO: 4 and corresponds to positions 250-273 of SEQ ID NO:5.

Burrel et al (supra) assessed the levels of polymorphism in the US28 protein amongst HCMV clinical strains, and concluded that the level of polymorphisms for US28 amongst clinical strains is higher than the level of polymorphisms previously reported for other HCMV-encoded proteins, such as UL97 phosphotransferase and UL44 processivity factor, although this polymorphism does not significantly vary according to HCMV susceptibility or resistance to currently approved antiviral drugs (i.e., GCV, FOS, and CDV), supporting therefore the idea that HCMV encoded US28 chemokine receptor may constitute a promising viral target for anti-HCMV drugs, especially in case of HCMV resistance.

A US28-focussed approach was taken by the authors of WO 2019/1.51865, as also reported in the equivalent journal article De Groof et al, 2019, Mol. Pharmaceutics, 16: 3145-3156. The authors reported that they had generated single heavy chain variable domain antibodies (VHH), exemplified by a particular VHH referred to as VUN100 (SEQ ID NO: 60 of the present application), that was said to specifically detect US28 in glioblastoma (GBM) tissues and inhibit ligand-dependent and constitutive US28 activity, and which the authors reported to consequently impair US28-dependent GBM growth in vitro and in vivo in an orthotopic xenograft model.

VUN100 was shown to bind to a discontinuous epitope, which comprise multiple binding positions within the N-terminal extracellular region of US28 (the complete N-terminal extracellular region, also referred to herein as ECD1, corresponds to positions 1-37), and further influenced by the presence of the third extracellular loop (ECL3, positions 250-273, also referred to herein as ECD4) of US28, as discussed in Example 3 of WO 2019/151865 (page 36, lines 11-32) and the legend to FIG. 2 of De Groof et at, 2019 (supra). De Groof (2019, supra) identified that removal of the N-terminal amino acid positions 1-22 results in no binding, and further identified that VUN100 does not bind to amino acid positions 11-15, but that amino acid position 16 is required. Furthermore, the results in relation to the binding of VUN100 to the different HCMV strains in FIG. 8D of WO 2019/151865 show a difference in binding between the VHL/E, Merlin and TB40/E strains of HCMV, with binding being particularly reduced in strain TB40/E (B1 type) at around only half the level of binding observed against the Merlin strain. US28 as encoded by the TB40/E strain differs from US28 as encoded by both of the Merlin and VHL/E strains at positions 8, 18 and 19 within the N-terminus (see Table 3 of the present application), suggesting that one or more of these positions (and potentially other amino acids in the 1-10 region and 16-22 region) also form part of the epitope bound by VUN100. Therefore, VUN100 is most likely binding between amino acid positions 1-10 and 16-22 of the N-terminus. Furthermore, replacement of the ECD4 region with the corresponding region of CCR5 prevented binding of VUN100, meaning that its binding requires a discontinuous epitope between positions 1-22 (including at least 16, and one or more of 8, 18 and/or 19) of the N-terminus and amino acids within ECD4.

As noted above, the level of polymorphisms for US28 amongst clinical strains is higher than the level of polymorphisms previously reported for other HCMV-encoded proteins (Eurrel et al, supra) and numerous N-terminal polymorphisms of US28 have been reported (Goffard et al., 2006, supra; Arav-Boger et al., 2002, supra). As determined by the applicant, and shown in Table 3 of the present application, the regions of the N-terminal of US28 bound by VUN100 are particularly susceptible to inter-strain polymorphisms.

The present inventor therefore considered the possibility that the presence of high levels of polymorphisms in particular parts of the N-terminal region of US28 and the third extracellular loop (“ECD4”), as bound by VUN100, may render the VUN100 VHH molecule incapable of maintaining binding characteristics against US28 consistently to the forms of US28 encoded by different HCMV strains. When considered in that context, as noted above, the binding of VUN100 to the different HCMV strains (FIG. 8D of WO 2019/151865) show a difference in binding between the VHL/E, Merlin and TB40/E strains of HCMV, with binding being particularly reduced in strain TB40/E (B1 type) at around only half the level of binding observed against the Merlin strain. Moreover, further characterisation of VUN100 is reported in a pre-printed article available online by De Groof et al, 2020 (doi:https://doi.org/10.1101/2020.05.12.071860), wherein FIG. 2 of the supplementary data gives the results of the % induced IE expression in the nucleus of CD14+ monocytes bound by VUN100. All cells tested were from HCMV seropositive individuals, and confirmed to be latently infected with HCMV, although the strain(s) of HCMV infecting each donor were undetermined. The level of IE expression induced by VUN100 binding to these HCMV-positive CD14+ cells from each of the four different patients varied substantially, with the reported figures being 57%, 33%, 22% and 4% (a range of difference of greater than 14-fold), respectively for cells from donors 1-4. This high level of response variability following the binding of VUN100 to the confirmed HCMV-positive cells seems to be most likely due to the infection of each of the donors with different HCMV strains, and thus a strong indication that the binding ability of VUN100 will vary considerably between different strains of HCMV. The same figure also shows high levels of response variability (in excess of 7-fold levels of difference) following the binding of a bivalent form of VUN100 (termed VUN100b by De Groof et al, 2020 (supra) as represented by SEQ ID NO: 63 of the present application) to the same group of HCMV-positive cells from the donors 1-4, again providing results indicative of strain-specific binding sensitivities. De Groof et al, 2021, Pharmacol Rev 73:828-846, also describes how serial passage of HCMV results in the development of resistant mutants with a truncated US28, having a premature stop codon in the extracellular loop 3, that results in reduced surface expression of US28. Such resistance may have adverse implications for VUN100, which relies on a partial epitope in the third extracellular loop.

Moreover, it is noted that an assay to compare the binding of the VUN100 Ab to US28-expressing HEK293T membranes versus to mock transfected HEK293T membranes showed that 20% of the mock transfected cells were bound by VUN100, and it only achieved a relative specificity score of 4 for the US28-expressing HEK293T membranes (De Groof et al., 2019 (supra) in their supporting information, FIG. S1.A thereof, and Table 2 of the present application). The apparent ability to distinguish between US28-expressing and US28-negative cells, with a specificity score of only around 4, particularly in conjunction with such high off-target binding to the mock cells, is potentially sub-optimal, and raises concerns about the ability of VUN100 to provide specifically targeted effects to HCMV-infected cells, whilst avoiding unacceptable levels of off-target side effects in healthy cells. It also raises concerns about the ability of VUN100 to be useful in the context of reliably identifying US28-expressing cells (in particular, HCMV-infected cells) in assays, including diagnostic assays, such as for use in immunohistochemistry (IHC). Furthermore, in a further publication, it has been acknowledged that the potency of US28-targeting nanobodies needs to be validated in a viral and potentially in vivo setting (De Groof et al., 2021, supra).

The provision of binding molecules having a substantially greater ability than VUN100 to bind specifically to US28-expressing cells (in particular, HCMV-infected cells) and/or to minimize off-target binding to cells that do not express US28 (in particular, cells that are not HCMV infected), both in vivo and/or when used in assays, including IHC, would be highly desirable.

In order to make use of US28 as a therapeutic target, it is important to overcome one or more of the most important obstacles, described above, including the relatively high levels of non-specific binding activity that has been reported for the art-known VUN100 molecule, the relatively high off-target binding activity observed for VUN100, and/or to provide US28-binding molecules that overcome obstacles of strain diversity, viral mutations, mutagenic drift and the ability of the virus to hide from the immune system during viral latency.

It is therefore an object of the present invention to provide binding molecules which can bind highly specifically to biological materials that express US28 and/or which are positive for HCMV infection, compared to healthy human cells which should be much lower and/or to minimise the absolute levels of off-target binding to healthy human cells. For example, the provision of binding molecules that provide, or direct, a cytotoxic effect to the cells to which the binding molecules become bound are of great interest for combatting HCMV infections and conditions associated therewith, and it is an object of the invention to provide binding molecules that can target these effects in a way that minimises or avoids unacceptable (e.g. therapeutically-unacceptable) levels of off-target cytotoxic effects in healthy human cells. In particular, it is one of the objects of the present invention to provide binding molecules having a higher level of specificity for US28 and/or HCMV-infected cells than the VUN100 Ab of WO 2019/151865 and De Groof et al, 2019 (supra) and/or than the VUN100b bivalent molecule of De Groof et al, 2020 (supra), when assessed for specificity in binding to biological materials (e.g. cells) that express US28 and/or which are positive for HCMV infection compared to biological materials (e.g. equivalent cells) that do not express US28 and which are not positive for HCMV infection.

It is another object of the present invention to provide binding molecules against US28 that are specific for the binding of biological materials that express US28 and/or which are positive for HCMV infected US28-expressing cells, compared to corresponding biological materials that do not express US28 (such as healthy human cells), and/or which display absolute levels of off-target binding to healthy human cells that are markedly lower than the VUN100 Ab molecules as noted above.

It is a further object of the present invention to provide binding molecules which are strain agnostic, and ideally therefore capable of targeting all, or substantially all, HCMV infections irrespective of the strain, or combination of strains, of HCMV present and/or capable of providing an ongoing effect during the course of treatment of HCMV infections, despite the possibility of the rise of one or more HCMV mutations in the infecting strain(s) within the individual(s) being treated. In particular, binding molecules against US28 that have binding characteristics that show a greater degree of strain agnostic binding than the VUN100 Ab are of particular interest.

### SUMMARY OF THE INVENTION

The present invention provides binding molecules having one or more (preferably all) of highly specific binding to the US28 protein of human cytomegalovirus (HCMV), very low levels of non-specific binding to healthy (non-infected) cells, and/or a strain-agnostic binding ability, as well as nucleic acid molecules encoding the said binding molecules.

The binding molecules of the present invention are designed to bind, and show binding specific for, a first epitope within a polypeptide consisting of the amino acid sequence of TDVLNQSKPVTL (SEQ ID NO: 177) within the N-terminus (also referred to herein as extracellular domain 1 (ECD1)) of a US28 protein of human cytomegalovirus (HCMV). The applicant has surprisingly identified that, unlike many of the areas of the N-terminal region (ECD1) of the US28 protein of HCMV, the amino acid sequence of TDVLNQSKPVTL (SEQ ID NO: 177) is highly conserved between all known HCMV strains, and furthermore is suitably immunogenic and suitable to use as an antigen for generating anti-US28 antibodies that have one or more (such as all) of: improved strain agnostic binding properties, improved (i.e. relatively higher) binding specificity, and/or improved (i.e. relatively lower) off-target binding activity, for example as compared to the monovalent and bivalent VUN100 antibodies of the prior art as discussed above.

The applicant has also surprisingly identified that the ECD3 of the US28 protein of HCMV is highly conserved between all known HCMV strains (possessing only a single position polymorphism, as discussed below), and furthermore is suitably immunogenic and suitable to use as an antigen for generating anti-US28 antibodies that have one or more (such as all) of: improved strain agnostic binding properties, improved (i.e. relatively higher) binding specificity, and/or improved (i.e. relatively lower) off-target binding activity, for example as compared to the monovalent and bivalent VUN100 antibodies of the prior art as discussed above. Accordingly, optionally, the binding molecules of the present invention are also designed to bind, and show binding specific for, a second epitope within extracellular domain 3 (ECD3) of a US28 protein of HCMV. Said binding molecule, having binding specificity against both the first and second epitopes may be in a multispecific format.

In a further option, a binding molecule of the present invention having binding specificity to a first epitope within a polypeptide consisting of the amino acid sequence of SEQ ID NO: 177 within the ECD1 of a US28 protein of HCMV, is a first binding molecule that is formulated with a second binding molecule that has binding specificity to a second epitope within ECD3 of a US28 protein of HCMV, wherein said second epitope is preferably as described further herein.

The binding molecules of the present invention have been demonstrated to have excellent binding properties, including those described above, and as further described herein. For example, the binding molecules of the present invention have also surprisingly been demonstrated to provide particularly advantageous binding specificity for aggressive and/or metastasizing HCMV-infected cancers, including breast cancers.

In certain preferred embodiments, the binding molecule is selected from an antibody (including, for example, a BiTE antibody) and a chimeric antigen receptor (CAR), or functional variants, fragments, fusion proteins, and/or conjugates thereof, as well as nucleic acid molecules encoding the same. Said binding molecule may, for example, include or be bound to a cytotoxic component or other effector component, having the ability to exert an influence on (such as to inhibit or kill) any cells bound by the binding molecule. Said binding molecule may, for example, include or be bound to a component that can recruit other agents (e.g. other proteins, drugs, cells or any other substance of choice) in such a way that the recruited agent has a specifically-targeted ability to exert an influence on (such as to inhibit or kill) cells bound by the binding molecule; a non-limiting example therefore is a BiTE molecule, which possesses the ability to recruit a T-cell to act upon cells bound by said BiTE. Also provided are cells expressing said binding molecules, including examples in which the binding molecule is a CAR, and said cells may be CAR-expressing cells, including CAR-T cells, CAR-NK cells, and CAR-M cells.

These, and further disclosures of the present invention are described in more detail by the following description and the appended claims and figures.

Accordingly, a first aspect of the present invention provides binding molecule, comprising one or more polypeptide chains, said binding molecule having binding specificity to at least a first epitope within a polypeptide consisting of the amino acid sequence of TDVLNQSKPVTL (SEQ ID NO: 177) within ECD1 of a US28 protein of human cytomegalovirus (HCMV).

Optionally, the binding molecule of the first aspect of the invention also has binding specificity to a second epitope within extracellular domain 3 (ECD3) of a US28 protein of HCMV (and/or wherein said binding molecule is a first binding molecule that is formulated with a second binding molecule that has binding specificity to a second epitope within ECD3 of a US28 protein of HCMV). For example, the binding molecule of the first aspect of the invention may be a multispecific (for example, bispecific or trispecific) binding molecule that comprises one or more regions with binding specificity to the first epitope within a polypeptide consisting of the amino acid sequence of SEQ ID NO: 177 within ECD1 and further comprises one or more regions with binding specificity to the second epitope within ECD3, respectively of the US28 protein encoded by HCMV.

In accordance with the present invention, ECD1, SEQ ID NO: 177, and ECD3 of the US28 protein each comprises, consists essentially of, or consist of, an amino acid sequence presented in the US28 protein encoded by a strain of HCMV at positions corresponding to positions 1 to 37, 26 to 37, and 167 to 183, respectively, of the US28 protein encoded by the DB strain of human cytomegalovirus (HCMV) as set forth in SEQ ID NO: 5.

For example, and without limitation, a binding molecule of the first aspect of the present invention may be selected from an antibody or a chimeric antigen receptor (CAR).

The binding molecule of the first aspect of the present invention may, for example, have binding specificity to a first epitope present entirely within the amino acid sequence of SEQ ID NO: 177, and/or optionally the second epitope is present entirely within ECD3, of the US28 protein of HCMV.

The binding molecule of the first aspect of the present invention may, for example, have binding specificity to a first linear epitope within the amino acid sequence of SEQ ID NO: 177, and/or optionally to a second linear epitope within ECD3, of the US28 protein.

The binding molecule of the first aspect of the present invention may, for example, have a strain agnostic binding specificity to an epitope within the amino acid sequence of SEQ ID NO: 177 in ECD1 and/or ECD3 of a US28 protein of HCMV. For example, the binding molecule may have:
a binding specificity to a first epitope within the amino acid sequence of SEQ ID NO: 177 of ECD1 of a US28 protein of HCMV, wherein the binding specificity is agnostic to two or more (such as all) of HCMV strains, for example agnostic to two or more variant strains with different sequences within the N-terminus, such as multiple HCMV strains that show inter-strain sequence variation in one or more positions in the region of positions 1 to 25 of ECD1 (such as any one or more of positions 8, 15, 18, 19, 24, 25, including but not limited to the sequence variations listed in Table 3 of the present application) for example wherein the multiple strains are two, three, four, five, six, seven, eight, nine or ten different strains selected from the group consisting of DB, Toledo, Towne, VR1814, TB40/E, Merlin, AD169, VHL/E, Davis and BL; and/or
the binding molecule may have a binding specificity to a second epitope within ECD3 of a US28 protein of HCMV, wherein the binding specificity is agnostic to two or more (such as all) of HCMV strains, for example agnostic to 4D-variant strains and 4N-variant strains (each as described further herein); and
in either case, optionally two or more (such as all) HCMV strains selected from the group consisting of DB, Towne, AF1, VHL/E, AD169, BL, DAVIS, JP, Merlin, PH, TB40/E, Toledo, TR and VR1814 (FIX).

Accordingly, the binding molecule of the first aspect of the present invention may, for example, have binding specificity to a first epitope within the amino acid sequence of SEQ ID NO: 177 of ECD1 of the US28 protein of HCMV, irrespective of whether the ECD1 of the US28 protein comprises mutations of known variants, provided that the first epitope comprises the sequence of TDVLNQSKPVTL (SEQ ID NO: 177), as found in the ECD1 of US28 as encoded by the following HCMV strains: DB, Towne, AF1, VHL/E, AD169, BL, DAVIS, JP, Merlin, PH, TB40/E, Toledo, TR and VR1814 (FIX). The sequence of TDVLNQSKPVTL (SEQ ID NO: 177) is fully conserved among all the listed clinical strains and, to the applicant's knowledge, is conserved amongst all known HCMV strains.

Optionally, the binding molecule of the first aspect of the present invention may, for example, have binding specificity to a second epitope within ECD3 of the US28 protein of HCMV, irrespective of whether the ECD3 of the US28 protein comprises the sequence of a 4D-variant or a 4N-variant:
wherein the 4D-variant comprises the sequence of TKKDNQCMTDYDYLEVS (SEQ ID NO: 7) as found in ECD3 of US28 as encoded by a first group of HCMV strains, such as Towne, VR1814, TB40/E, Merlin, JP, Ad169, AF1, VHL/E, BL and DAVIS; and
wherein the 4N-variant comprises the sequence of TKKNNQCMTDYDYLEVS (SEQ ID NO: 6) as found in ECD3 of US28 as encoded by a second group of HCMV strains, such as Toledo, TR and DB strains.

By making use of the protocols described herein, the applicant has consistently and repeatedly generated numerous antibodies with binding specificity to a first epitope within the amino acid sequence of SEQ ID NO: 177 of ECD1 of the US28 protein of HCMV, and numerous antibodies with binding specificity to a second epitope within ECD3 of the US28 protein of HCMV, with one or more of the above-noted beneficial binding properties, including antibodies referred to herein by the following designations (the sequences of which are also provided below):

Exemplary antibodies against SEQ ID NO: 177: US28-4-4H3C3, US28-4-7B1F3, US28-4-2F5B11, US28-4-4A11A11, US28-4-2G2B3, US28-4-5D6H11, US28-4-9G3B9, US28-4-5E8F7, US28-4-6G3D6, and US28-4-5E1E4; and

Exemplary antibodies against ECD3: US28-13-5G6-1D3, US28-13-1C10-1C10, US28-13-1H3-1A10, US28-14-2C2-1G4, US28-13-1C10-1G9, and US28-14-4E4-1E8.

In one embodiment, the binding molecule of the first aspect of the present invention comprises one, two, three, four, five or six complementarity determining regions (CDRs) having binding specificity to the first epitope within a polypeptide consisting of the amino acid sequence of SEQ ID NO: 177, corresponding to any one, two, three, four, five or all six of the CDR sequences of antibody US28-4-4H3C3 (generally abbreviated herein to “4H13C3”) as defined by SEQ ID NOs: 196, 197, 198, 199, 200 and 201, respectively, and/or a functional variant of any one or more of said CDR sequences of antibody 4H3C3, and optionally wherein the binding molecule is selected from an antibody and a CAR.

Optionally, the binding molecule according to this embodiment may comprise:
(a) one, two, or all three, of the CDR 1, 2, and 3, sequences of the variable heavy chain (V_{H}) of antibody 4H3C3, as defined by SEQ ID NOs: 196, 197, and 198, respectively; and/or
(b) one, two, or all three, of the CDR 1, 2, and 3, sequences of the variable light chain (V_{L}) of antibody 4H3C3, as defined by SEQ ID NOs: 199, 200, and 201, respectively.

Additionally, or alternatively, in a further option the binding molecule according to this embodiment may comprise:
(a) at least one variable heavy chain (V_{H}) polypeptide having binding specificity to the first epitope within a polypeptide consisting of the amino acid sequence of SEQ ID NO: 177, wherein said V_{H} polypeptide comprises CDR 1, 2, and 3 sequences having the sequences of SEQ ID Nos: 196, 197, and 198, respectively, and optionally wherein the at least one variable heavy chain (V_{H}) polypeptide comprises, consists essentially of, or consists of, the sequence of SEQ ID NO: 1.87; and/or
(b) at least one variable light chain (V_{L}) polypeptide having binding specificity to the first epitope within a polypeptide consisting of the amino acid sequence of SEQ ID NO: 177, wherein said V_{L} polypeptide comprises CDR 1, 2 and 3 sequences having the sequences of SEQ ID NOs: 199, 200, and 201, respectively, and optionally wherein the variable light chain (V_{L}) polypeptide comprises, consists essentially of, or consists of, the sequence of SEQ ID NO: 189.

The following sequences of the 4H3C3 antibody are identified herein, with reference to the sequence identification numbers (SEQ ID NOs) described below:

| 4H3C3 | | DNA Sequence | Protein Sequence |
|---|---|---|---|
| Complete heavy chain | | SEQ ID NO: 178 | SEQ ID NO: 180 |
| (including leader sequence) | | | |
| Complete light chain | | SEQ ID NO: 179 | SEQ ID NO: 181 |
| (including leader sequence) | | | |
| V_{H} chain | Immature | SEQ ID NO: 182 | SEQ ID NO: 186 |
| | Mature | SEQ ID NO: 183 | SEQ ID NO: 187 |
| V_{L} chain | Immature | SEQ ID NO: 184 | SEQ ID NO: 188 |
| | Mature | SEQ ID NO: 185 | SEQ ID NO: 189 |

| | | |
|---|---|---|
| V_{H}-CDR1 | SEQ ID NO: 190 | SEQ ID NO: 196 |
| V_{H}-CDR2 | SEQ ID NO: 191 | SEQ ID NO: 197 |
| V_{H}-CDR3 | SEQ ID NO: 192 | SEQ ID NO: 198 |
| V_{L}-CDR1 | SEQ ID NO: 193 | SEQ ID NO: 199 |
| V_{L}-CDR2 | SEQ ID NO: 194 | SEQ ID NO: 200 |
| V_{L}-CDR3 | SEQ ID NO: 195 | SEQ ID NO: 201 |

In some embodiments, the 4H3C3 binding molecule is humanised (referred to herein as “a 4H3C3 humanised variant”) and, optionally, the binding molecule may comprise:
(a) V_{H}-CDR1 as described by SEQ ID NO: 196;
(b) V_{H}-CDR2 as described by any one of SEQ ID NOs: 197, 293 and 294;
(c) V_{H}-CDR3 as described by SEQ ID NO: 198;
(d) V_{L}-CDR1 as described by either SEQ ID NO: 199 or 295;
(e) V_{L}-CDR2 as described by either SEQ ID NO: 200 or 222; and/or
(f) V_{L}-CDR3 as described by SEQ ID NO: 201.

In some embodiments, the 4H3C3 binding molecule is humanised and, optionally, comprises:
(a) a variable heavy chain selected from the group consisting of SEQ ID NOs: 256-259; and/or
(b) a variable light chain selected from the group consisting of SEQ ID NOs: 260-263.

In another embodiment, the binding molecule of the first aspect of the present invention comprises one, two, three, four, five or six CDRs having binding specificity to the first epitope within a polypeptide consisting of the amino acid sequence of SEQ ID NO: 177, corresponding to any one, two, three, four, five or all six of the CDR sequences of antibody US28-4-7B1F3 (generally abbreviated herein to “7B1F3”) as defined by SEQ ID NOs: 219, 220, 221, 199, 222 and 223, respectively, and/or a functional variant of any one or more of said CDR sequences of antibody 7B1F3, and optionally wherein the binding molecule is selected from an antibody and a CAR.

Optionally, the binding molecule according to this embodiment may comprise:
(a) one, two, or all three, of the CDR 1, 2, and 3, sequences of the V_{H} of antibody 7B1F3, as defined by SEQ ID NOs: 219, 220, and 221, respectively; and/or
(b) one, two, or all three, of the CDR 1, 2, and 3, sequences of the V_{L} of antibody 7B1F3, as defined by SEQ ID NOs: 199, 222, and 223, respectively.

Additionally, or alternatively, in a further option the binding molecule according to this embodiment may comprise:
(a) at least one V_{H} polypeptide having binding specificity to the first epitope within a polypeptide consisting of the amino acid sequence of SEQ ID NO: 177, wherein said V_{H} polypeptide comprises CDR 1, 2, and 3 sequences having the sequences of SEQ ID NOs: 219, 220, and 221, respectively, and optionally wherein the at least one V_{H} polypeptide comprises, consists essentially of, or consists of, the sequence of SEQ ID NO: 211; and/or
(b) at least one V_{L} polypeptide having binding specificity to the first epitope within a polypeptide consisting of the amino acid sequence of SEQ ID NO: 177, wherein said V_{L} polypeptide comprises CDR 1, 2 and 3 sequences having the sequences of SEQ ID NOs: 199, 222, and 223, respectively, and optionally wherein the V_{L} polypeptide comprises, consists essentially of, or consists of, the sequence of SEQ ID NO: 213.

The following sequences of the 7B1F3 antibody are identified herein, with reference to the sequence identification numbers (SEQ ID NOs) described below: 7B1F3 DNA Sequence Protein Sequence

| 7B1F3 | | DNA Sequence | Protein Sequence |
|---|---|---|---|
| Complete heavy chain | | SEQ ID NO: 202 | SEQ ID NO: 204 |
| (including leader sequence) | | | |
| Complete light chain | | SEQ ID NO: 203 | SEQ ID NO: 205 |
| (including leader sequence) | | | |
| V_{H} chain | Immature | SEQ ID NO: 206 | SEQ ID NO: 210 |
| | Mature | SEQ ID NO: 207 | SEQ ID NO: 211 |
| V_{L} chain | Immature | SEQ ID NO: 208 | SEQ ID NO: 212 |
| | Mature | SEQ ID NO: 209 | SEQ ID NO: 213 |

| | | |
|---|---|---|
| V_{H}-CDR1 | SEQ ID NO: 214 | SEQ ID NO: 219 |
| V_{H}-CDR2 | SEQ ID NO: 215 | SEQ ID NO: 220 |
| V_{H}-CDR3 | SEQ ID NO: 216 | SEQ ID NO: 221 |
| V_{L}-CDR1 | SEQ ID NO: 251 | SEQ ID NO: 199 |
| V_{L}-CDR2 | SEQ ID NO: 217 | SEQ ID NO: 222 |
| V_{L}-CDR3 | SEQ ID NO: 218 | SEQ ID NO: 223 |

In some embodiments, the 7B1F3 binding molecule is humanised (referred to herein as “a 7B1F3 humanised variant”) and, optionally, the binding molecule may comprise:
(a) V_{H}-CDR1 as described by SEQ ID NO: 219;
(b) V_{H}-CDR2 as described by any one of SEQ ID NOs: 220, 296 and 297;
(c) V_{H}-CDR3 as described by either SEQ ID NO: 221 or 298;
(d) V_{L}-CDR1 as described by either SEQ ID NO: 199 or 295;
(e) V_{L}-CDR2 as described by either SEQ ID NO: 222; and/or
(f) V_{L}-CDR3 as described by SEQ ID NO: 223.

In some embodiments, the 7B1F3 binding molecule is humanised and, optionally, comprises:
(a) a variable heavy chain selected from the group consisting of SEQ ID NOs: 264-267, 270 or 271; and/or
(b) a variable light chain selected from the group consisting of SEQ ID NOs: 268, 261, 269 and 263.

In some preferable embodiments, the variable heavy chain of the 7B1F3 binding molecule has been mutated to remove a glycosylation site in the V_{H}-CDR3 domain. Preferably, the V_{H}-CDR3 is selected from SEQ ID NOs: 270 or 271.

In another embodiment, the binding molecule of the first aspect of the present invention comprises one, two, three, four, five or six CDRs having binding specificity to the first epitope within a polypeptide consisting of the amino acid sequence of SEQ ID NO: 177, corresponding to any one, two, three, four, five or all six of the CDR sequences of antibody US28-4-2F5B11 (generally abbreviated herein to “2F5B11”) as defined by SEQ ID NOs: 242, 243, 244, 199, 245 and 246, respectively, and/or a functional variant of any one or more of said CDR sequences of antibody 2F5B11, and optionally wherein the binding molecule is selected from an antibody and a CAR.

Optionally, the binding molecule according to this embodiment may comprise:
(a) one, two, or all three, of the CDR 1, 2, and 3, sequences of the V_{H} of antibody 2F5B11, as defined by SEQ ID NOs: 242, 243, and 244, respectively; and/or
(b) one, two, or all three, of the CDR 1, 2, and 3, sequences of the V_{L} of antibody 2F5B11, as defined by SEQ ID NOs: 199, 245, and 246, respectively.

Additionally, or alternatively, in a further option the binding molecule according to this embodiment may comprise:
(a) at least one V_{H} polypeptide having binding specificity to the first epitope within a polypeptide consisting of the amino acid sequence of SEQ ID NO: 177, wherein said V_{H} polypeptide comprises CDR 1, 2, and 3 sequences having the sequences of SEQ ID NOs: 242, 243, and 244, respectively, and optionally wherein the at least one V_{H} polypeptide comprises, consists essentially of, or consists of, the sequence of SEQ ID NO: 233; and/or
(b) at least one V_{L} polypeptide having binding specificity to the first epitope within a polypeptide consisting of the amino acid sequence of SEQ ID NO: 177, wherein said V_{L} polypeptide comprises CDR 1, 2 and 3 sequences having the sequences of SEQ ID NOs: 199, 245, and 246, respectively, and optionally wherein the V_{L} polypeptide comprises, consists essentially of, or consists of, the sequence of SEQ ID NO: 235.

The following sequences of the 2F5311 antibody are identified herein, with reference to the sequence identification numbers (SEQ ID NOs) described below:

| 2F5B11 | | DNA Sequence | Protein Sequence |
|---|---|---|---|
| Complete heavy chain | | SEQ ID NO: 224 | SEQ ID NO: 226 |
| (including leader sequence) | | | |
| Complete light chain | | SEQ ID NO: 225 | SEQ ID NO: 227 |
| (including leader sequence) | | | |
| V_{H} chain | Immature | SEQ ID NO: 228 | SEQ ID NO: 232 |
| | Mature | SEQ ID NO: 229 | SEQ ID NO: 233 |
| V_{L} chain | Immature | SEQ ID NO: 230 | SEQ ID NO: 234 |
| | Mature | SEQ ID NO: 231 | SEQ ID NO: 235 |

| | | |
|---|---|---|
| V_{H}-CDR1 | SEQ ID NO: 236 | SEQ ID NO: 242 |
| V_{H}-CDR2 | SEQ ID NO: 237 | SEQ ID NO: 243 |
| V_{H}-CDR3 | SEQ ID NO: 238 | SEQ ID NO: 244 |
| V_{L}-CDR1 | SEQ ID NO: 239 | SEQ ID NO: 199 |
| V_{L}-CDR2 | SEQ ID NO: 240 | SEQ ID NO: 245 |
| V_{L}-CDR3 | SEQ ID NO: 241 | SEQ ID NO: 246 |

In another embodiment, the binding molecule of the first aspect of the present invention comprises one, two, three, four, five or six CDRs having binding specificity to the first epitope within a polypeptide consisting of the amino acid sequence of SEQ ID NO: 177, corresponding to any one, two, three, four, five or all six of the following consensus CDR sequences (wherein replacement amino acids for a particular position are indicated in parenthesis, and * indicates an absence of an amino acid at that position):

(a) V_{H}-CDR1 corresponding to
(SEQ ID NO: 247)
(S/N)YW(M/I)H;
(b) V_{H}-CDR2 corresponding to
(SEQ ID NO: 248)
Y(I/V)NP(S/R/N)T(G/A)Y(A/T)E(Y/F)NQKF(K/M)D
or
(SEQ ID NO: 299)
Y(I/V)NP(S/R/N)T(G/A)Y(A/T)E(Y/F)NQ(K/Q)
(F/L)(K/M/Q)(D/G);
(c) V_{H}-CDR3 corresponding to
(SEQ ID NO: 249)
(L/I)(R/L)(F/N/S)(G/D)(S/*/R)(S/T)GFAY
or
(SEQ ID NO: 300)
(L/I)(R/L)(F/N/S/Q)(G/D)(S/*/R)(S/T)GFAY;
(d) V_{L}-CDR1 corresponding to
(SEQ ID NO: 199)
KSSQSLLYSSNQKNYLA
or
(SEQ ID NO: 301)
(K/R)SSQSLLYSSNQKNYLA;
(e) V_{L}-CDR2 corresponding to
(SEQ ID NO: 250)
WAST(W/R)E(S/Y);
and/or
(f) V_{L}-CDR3 corresponding to
(SEQ ID NO: 201)
QQYYSFPLT.

Functional variants of any one or more of the V_{H}-CDR1, V_{H}-CDR2, V_{H}-CDR3, V_{L}-CDR1, V_{L}-CDR2 and/or V_{L}-CDR3 sequences having binding specificity to the first epitope within a polypeptide consisting of the amino acid sequence of SEQ ID NO: 177, as defined above for any of antibodies 4H3C3, 7B1F3, and/or 2F5B11 may optionally comprise a sequence that possess the corresponding consensus sequence as set forth above for the V_{H}-CDR1, V_{H}-CDR2, V_{H}-CDR3, V_{L}-CDR1, V_{L}-CDR2 and/or V_{L}-CDR3 sequences, by SEQ ID NOs: 247, 248 or 299, 249 or 300, 199 or 301, 250, and 201, respectively.

Optionally, the binding molecule of the first aspect of the present invention further comprises one, two, three, four, five or six complementarity determining regions (CDRs) having binding specificity to the second epitope within ECD3 of a US28 protein of HCMV, corresponding to any one, two, three, four, five or all six of the CDR sequences of antibody 13-5G6-1D3 (generally abbreviated herein to “1D3”) as defined by SEQ ID NOs: 8, 9, 10, 14, 15 and 16, respectively, and/or a functional variant of any one or more of said CDR sequences of antibody 1D3, and optionally wherein the binding molecule is selected from an antibody and a CAR.

For example, the binding molecule according to this option of the first aspect of the present invention may further comprise (a) one, two, or all three, of the CDR 1, 2, and 3, sequences of the variable heavy chain (V_{H}) of antibody 1D3, as defined by SEQ ID NOs: 8, 9, and 10, respectively; and/or (b) one, two, or all three, of the CDR 1, 2, and 3, sequences of the variable light chain (V_{L}) of antibody 1D3, as defined by SEQ ID NOs: 14, 15, and 16, respectively.

Additionally, or alternatively, in this option the binding molecule may further comprise: (a) at least one variable heavy chain (V_{H}) polypeptide that comprises CDR 1, 2, and 3 sequences having the sequences of SEQ ID Nos: 8, 9, and 10, respectively, and optionally wherein the at least one variable heavy chain (V_{H}) polypeptide comprises, consists essentially of, or consists of, the sequence of SEQ ID NO: 12; and/or (b) at least one variable light chain (V_{L}) polypeptide that comprises CDR 1, 2 and 3 sequences having the sequences of SEQ ID NOs: 14, 15, and 16, respectively, and optionally wherein the variable light chain (V_{L}) polypeptide comprises, consists essentially of, or consists of, the sequence of SEQ ID NO:18.

The following sequences of the US28-13-5G6-1D3 antibody are identified herein, with reference to the sequence identification numbers (SEQ ID NOs) described below:

| 1D3 | | DNA Sequence | Protein Sequence |
|---|---|---|---|
| Complete heavy chain | | SEQ ID NO: 34 | SEQ ID NO: 20 |
| (including leader sequence) | | | |
| Complete light chain | | SEQ ID NO: 35 | SEQ ID NO: 21 |
| (including leader sequence) | | | |
| V_{H} chain | Immature | SEQ ID NO: 25 | SEQ ID NO: 11 |
| | Mature | SEQ ID NO: 26 | SEQ ID NO: 12 |
| V_{L} chain | Immature | SEQ ID NO: 31 | SEQ ID NO: 17 |
| | Mature | SEQ ID NO: 32 | SEQ ID NO: 18 |

| | | |
|---|---|---|
| V_{H}-CDR1 | SEQ ID NO: 22 | SEQ ID NO: 8 |
| V_{H}-CDR2 | SEQ ID NO: 23 | SEQ ID NO: 9 |
| V_{H}-CDR3 | SEQ ID NO: 24 | SEQ ID NO: 10 |
| V_{L}-CDR1 | SEQ ID NO: 28 | SEQ ID NO: 14 |
| V_{L}-CDR2 | SEQ ID NO: 29 | SEQ ID NO: 15 |
| V_{L}-CDR3 | SEQ ID NO: 30 | SEQ ID NO: 16 |

In another option, the binding molecule of the first aspect of the present invention further comprises one, two, three, four, five or six CDRs having binding specificity to the second epitope within ECD3 of a US28 protein of HCMV, corresponding to any one, two, three, four, five or all six of the CDR sequences of antibody 13-1C10-1C10 (generally abbreviated herein to “1C1G”) as defined by SEQ ID NOs: 112, 113, 114, 117, 83 and 118, respectively, and/or a functional variant of any one or more of said CDR sequences of antibody 1C10, and optionally wherein the binding molecule is selected from an antibody and a CAR.

For example, the binding molecule according to this option of the first aspect of the present invention may further comprise (a) one, two, or all three, of the CDR 1, 2, and 3, sequences of the V_{H} of antibody 13-1C10-1C10, as defined by SEQ ID NOs: 112, 113, and 114, respectively; and/or (b) one, two, or all three, of the CDR 1, 2, and 3, sequences of the V_{L} of antibody 13-1C10-1C10, as defined by SEQ ID NOs: 117, 83, and 118, respectively.

Additionally, or alternatively, in this option the binding molecule according to this embodiment may further comprise: (a) at least one V_{H} polypeptide that comprises CDR 1, 2, and 3 sequences having the sequences of SEQ ID NOs: 112, 113, and 114, respectively, and optionally wherein the at least one V_{H} polypeptide comprises, consists essentially of, or consists of, the sequence of SEQ ID NO: 104; and/or (b) at least one V_{L} polypeptide that comprises CDR 1, 2 and 3 sequences having the sequences of SEQ ID NOs: 117, 83, and 118, respectively, and optionally wherein the V_{L} polypeptide comprises, consists essentially of, or consists of, the sequence of SEQ ID NO: 108.

The following sequences of the US28-13-1C10-1C10 antibody are identified herein, with reference to the sequence identification numbers (SEQ ID NOs) described below:

| 1C10 | | DNA Sequence | Protein Sequence |
|---|---|---|---|
| Complete heavy chain | | SEQ ID NO: 155 | SEQ ID NO: 157 |
| (including leader sequence) | | | |
| Complete light chain | | SEQ ID NO: 156 | SEQ ID NO: 158 |
| (including leader sequence) | | | |
| V_{H} chain | Immature | SEQ ID NO: 101 | SEQ ID NO: 103 |
| | Mature | SEQ ID NO: 102 | SEQ ID NO: 104 |
| V_{L} chain | Immature | SEQ ID NO: 105 | SEQ ID NO: 107 |
| | Mature | SEQ ID NO: 106 | SEQ ID NO: 108 |

| | | |
|---|---|---|
| V_{H}-CDR1 | SEQ ID NO: 109 | SEQ ID NO: 112 |
| V_{H}-CDR2 | SEQ ID NO: 110 | SEQ ID NO: 113 |
| V_{H}-CDR3 | SEQ ID NO: 111 | SEQ ID NO: 114 |
| V_{L}-CDR1 | SEQ ID NO: 115 | SEQ ID NO: 117 |
| V_{L}-CDR2 | SEQ ID NO: 80 | SEQ ID NO: 83 |
| V_{L}-CDR3 | SEQ ID NO: 116 | SEQ ID NO: 118 |

In another option, the binding molecule of the first aspect of the present invention further comprises one, two, three, four, five or six CDRs having binding specificity to the second epitope within ECD3 of a US28 protein of HCMV, corresponding to any one, two, three, four, five or all six of the CDR sequences of antibody 13-1H3-1A10 (generally abbreviated herein to “1A10”) as defined by SEQ ID NOs: 112, 113, 114, 117, 83 and 118, respectively, and/or a functional variant of any one or more of said CDR sequences of antibody 1A10, and optionally wherein the binding molecule is selected from an antibody and a CAR.

For example, the binding molecule according to this option of the first aspect of the present invention may further comprise (a) one, two, or all three, of the CDR 1, 2, and 3, sequences of the V_{H} of antibody 13-1H3-1A10, as defined by SEQ ID NOs: 112, 113, and 114, respectively; and/or (b) one, two, or all three, of the CDR 1, 2, and 3, sequences of the V_{L} of antibody 13-1H3-1A10, as defined by SEQ ID NOs: 117, 83, and 118, respectively.

Additionally, or alternatively, in this option the binding molecule according to this embodiment may further comprise: (a) at least one V_{H} polypeptide that comprises CDR 1, 2, and 3 sequences having the sequences of SEQ ID NOs: 112, 113, and 114, respectively, and optionally wherein the at least one V_{H} polypeptide comprises, consists essentially of, or consists of, the sequence of SEQ ID NO: 122; and/or (b) at least one V_{L} polypeptide that comprises CDR 1, 2 and 3 sequences having the sequences of SEQ ID NOs: 117, 83, and 118, respectively, and optionally wherein the V_{L} polypeptide comprises, consists essentially of, or consists of, the sequence of SEQ ID NO: 126.

The following sequences of the US28-13-1H3-1A10 antibody are identified herein, with reference to the sequence identification numbers (SEQ ID NOs) described below:

| 1A10 | | DNA Sequence | Protein Sequence |
|---|---|---|---|
| Complete heavy chain | | SEQ ID NO: 159 | SEQ ID NO: 161 |
| (including leader sequence) | | | |
| Complete light chain | | SEQ ID NO: 160 | SEQ ID NO: 162 |
| (including leader sequence) | | | |
| V_{H} chain | Immature | SEQ ID NO: 119 | SEQ ID NO: 121 |
| | Mature | SEQ ID NO: 120 | SEQ ID NO: 122 |
| V_{L} chain | Immature | SEQ ID NO: 123 | SEQ ID NO: 125 |
| | Mature | SEQ ID NO: 124 | SEQ ID NO: 126 |

| | | |
|---|---|---|
| V_{H}-CDR1 | SEQ ID NO: 109 | SEQ ID NO: 112 |
| V_{H}-CDR2 | SEQ ID NO: 110 | SEQ ID NO: 113 |
| V_{H}-CDR3 | SEQ ID NO: 111 | SEQ ID NO: 114 |
| V_{L}-CDR1 | SEQ ID NO: 115 | SEQ ID NO: 117 |
| V_{L}-CDR2 | SEQ ID NO: 80 | SEQ ID NO: 83 |
| V_{L}-CDR3 | SEQ ID NO: 116 | SEQ ID NO: 118 |

In another option, the binding molecule of the first aspect of the present invention further comprises one, two, three, four, five or six CDRs having binding specificity to the second epitope within ECD3 of a US28 protein of HCMV, corresponding to any one, two, three, four, five or all six of the CDR sequences of antibody 13-1C10-1G9 (generally abbreviated herein to “1G9”) as defined by SEQ ID NOs: 76, 77, 78, 82, 83 and 84, respectively, and/or a functional variant of any one or more of said CDR sequences of antibody 1G9, and optionally wherein the binding molecule is selected from an antibody and a CAR.

For example, the binding molecule according to this option of the first aspect of the present invention may further comprise (a) one, two, or all three, of the CDR 1, 2, and 3, sequences of the V_{H} of antibody 13-1C10-1G9, as defined by SEQ ID NOs: 76, 77, and 78, respectively; and/or (b) one, two, or all three, of the CDR 1, 2, and 3, sequences of the V_{L} of antibody 13-1C10-1G9, as defined by SEQ ID NOs: 82, 83, and 84, respectively.

Additionally, or alternatively, in this option the binding molecule according to this embodiment may further comprise: (a) at least one V_{H} polypeptide that comprises CDR 1, 2, and 3 sequences having the sequences of SEQ ID NOs: 76, 77, and 78, respectively, and optionally wherein the at least one V_{H} polypeptide comprises, consists essentially of, or consists of, the sequence of SEQ ID NO: 68; and/or (b) at least one V_{L} polypeptide that comprises CDR 1, 2 and 3 sequences having the sequences of SEQ ID NOs: 82, 83, and 84, respectively, and optionally wherein the V_{L} polypeptide comprises, consists essentially of, or consists of, the sequence of SEQ ID NO: 72.

The following sequences of the US28-13-1C10-1G9 antibody are identified herein, with reference to the sequence identification numbers (SEQ ID NOs) described below:

| 1G9 | | DNA Sequence | Protein Sequence |
|---|---|---|---|
| Complete heavy chain | | SEQ ID NO: 147 | SEQ ID NO: 149 |
| (including leader sequence) | | | |
| Complete light chain | | SEQ ID NO: 148 | SEQ ID NO: 150 |
| (including leader sequence) | | | |
| V_{H} chain | Immature | SEQ ID NO: 65 | SEQ ID NO: 67 |
| | Mature | SEQ ID NO: 66 | SEQ ID NO: 68 |
| V_{L} chain | Immature | SEQ ID NO: 69 | SEQ ID NO: 71 |
| | Mature | SEQ ID NO: 70 | SEQ ID NO: 72 |

| | | |
|---|---|---|
| V_{H}-CDR1 | SEQ ID NO: 73 | SEQ ID NO: 76 |
| V_{H}-CDR2 | SEQ ID NO: 74 | SEQ ID NO: 77 |
| V_{H}-CDR3 | SEQ ID NO: 75 | SEQ ID NO: 78 |
| V_{L}-CDR1 | SEQ ID NO: 79 | SEQ ID NO: 82 |
| V_{L}-CDR2 | SEQ ID NO: 80 | SEQ ID NO: 83 |
| V_{L}-CDR3 | SEQ ID NO: 81 | SEQ ID NO: 84 |

In another option, the binding molecule of the first aspect of the present invention further comprises one, two, three, four, five or six CDRs having binding specificity to the second epitope within ECD3 of a US28 protein of HCMV, corresponding to any one, two, three, four, five or all six of the CDR sequences of antibody 14-4E4-1E8 (generally abbreviated herein to “IE8”) as defined by SEQ ID NOs: 76, 95, 96, 82, 99 and 100, respectively, and/or a functional variant of any one or more of said CDR sequences of antibody 1E8, and optionally wherein the binding molecule is selected from an antibody and a CAR.

For example, the binding molecule according to this option of the first aspect of the present invention may further comprise (a) one, two, or all three, of the CDR 1, 2, and 3, sequences of the V_{H} of antibody 14-4E4-1E8, as defined by SEQ ID NOs: 76, 95, and 96, respectively; and/or (b) one, two, or all three, of the CDR 1, 2, and 3, sequences of the V_{L} of antibody 14-4E4-1E8, as defined by SEQ ID NOs: 82, 99, and 100, respectively.

Additionally, or alternatively, in this option the binding molecule according to this embodiment may further comprise: (a) at least one V_{H} polypeptide that comprises CDR 1, 2, and 3 sequences having the sequences of SEQ ID NOs: 76, 95, and 96, respectively, and optionally wherein the at least one V_{H} polypeptide comprises, consists essentially of, or consists of, the sequence of SEQ ID NO: 88; and/or (b) at least one V_{L} polypeptide that comprises CDR 1, 2 and 3 sequences having the sequences of SEQ ID NOs: 82, 99, and 100, respectively, and optionally wherein the V_{L} polypeptide comprises, consists essentially of, or consists of, the sequence of SEQ ID NO: 92.

The following sequences of the US28-14-4E4-1E8 antibody are identified herein, with reference to the sequence identification numbers (SEQ ID NOs) described below:

| 1E8 | | DNA Sequence | Protein Sequence |
|---|---|---|---|
| Complete heavy chain | | SEQ ID NO: 151 | SEQ ID NO: 153 |
| (including leader sequence) | | | |
| Complete light chain | | SEQ ID NO: 152 | SEQ ID NO: 154 |
| (including leader sequence) | | | |
| V_{H} chain | Immature | SEQ ID NO: 85 | SEQ ID NO: 87 |
| | Mature | SEQ ID NO: 86 | SEQ ID NO: 88 |
| V_{L} chain | Immature | SEQ ID NO: 89 | SEQ ID NO: 91 |
| | Mature | SEQ ID NO: 90 | SEQ ID NO: 92 |

| | | |
|---|---|---|
| V_{H}-CDR1 | SEQ ID NO: 73 | SEQ ID NO: 76 |
| V_{H}-CDR2 | SEQ ID NO: 93 | SEQ ID NO: 95 |
| V_{H}-CDR3 | SEQ ID NO: 94 | SEQ ID NO: 96 |
| V_{L}-CDR1 | SEQ ID NO: 79 | SEQ ID NO: 82 |
| V_{L}-CDR2 | SEQ ID NO: 97 | SEQ ID NO: 99 |
| V_{L}-CDR3 | SEQ ID NO: 98 | SEQ ID NO: 100 |

In another option, the binding molecule of the first aspect of the present invention further comprises one, two, three, four, five or six CDRs having binding specificity to the second epitope within ECD3 of a US28 protein of HCMV, corresponding to any one, two, three, four, five or all six of the following consensus CDR sequences (wherein replacement amino acids for a particular position are indicated in parenthesis, and * indicates an absence of an amino acid at that position):

(a) V_{H}-CDR1 corresponding to
(SEQ ID NO: 167)
S(Y/H)A(M/L)S;
(b) V_{H}-CDR2 corresponding to
(SEQ ID NO: 168)
SISS(G/R)G(S/R)TYYPDSVKG;
(c) V_{H}-CDR3 corresponding to
(SEQ ID NO: 169)
GG(S/T)(T/R/H)(M/H/Y)(I/S)(T/Y)
(T/G)(G/N)(L/*)GF(A/D)(Y/F);
(d) V_{L}-CDR1 corresponding to
(SEQ ID NO: 170)
S(A/V)SSSVSYMH;
(e) V_{L}-CDR2 corresponding to
(SEQ ID NO: 171)
D(T/S)SKLAS;
and/or
(f) V_{L}-CDR3 corresponding to
(SEQ ID NO: 172)
QQW(S/T/*)SN(*/N)PP(I/L)T.

In another option, the binding molecule of the first aspect of the present invention further comprises one, two, three, four, five or six CDRs having binding specificity to the second epitope within ECD3 of a US28 protein of HCMV, corresponding to any one, two, three, four, five or all six of the following consensus CDR sequences (wherein replacement amino acids for a particular position are indicated in parenthesis, and * indicates an absence of an amino acid at that position):

(a) V_{H}-CDR1 corresponding to
(SEQ ID NO: 167)
S(Y/H)A(M/L)S;
(b) V_{H}-CDR2 corresponding to
(SEQ ID NO: 174)
SISS(G/R)GRTYYPDSVKG;
(c) V_{H}-CDR3 corresponding to
(SEQ ID NO: 175)
GG(S/T)(T/R/H)(M/H/Y)(I/S)(T/Y)
(T/G)(G/N)GF(A/D)(Y/F);
(d) V_{L}-CDR1 corresponding to
(SEQ ID NO: 170)
S(A/V)SSSVSYMH;
(e) V_{L}-CDR2 corresponding to
(SEQ ID NO: 171)
D(T/S)SKLAS;
and/or
(f) V_{L}-CDR3 corresponding to
(SEQ ID NO: 176)
QQW(T/*)SN(*/N)PPIT.

Functional variants of any one or more of the V_{H}-CDR1, V_{H}-CDR2, V_{H}-CDR3, V_{L}-CDR1, V_{L}-CDR2 and/or V_{L}-CDR3 sequences having binding specificity to the second epitope within ECD3 of the US28 protein of HCMV, as defined above for any of antibodies US28-13-5G6-1D3, 13-1C10-1C10, 13-1H3-1A10, 13-1C10-1G9, 14-4E4-1E8 may optionally comprise a sequence that possess the corresponding consensus sequences as set forth above in either of the two preceding paragraphs for the V_{H}-CDR1, V_{H}-CDR2, V_{H}-CDR3, V_{L}-CDR1, V_{L}-CDR2 and/or V_{L}-CDR3 sequences, respectively.

Sequences corresponding to the above-noted SEQ ID NOs are given in the section of this application entitled “Sequences”, which follows the Examples, in this application.

In certain embodiments, a binding molecule according to the first aspect of the present invention may be selected from the group consisting of:
(a) bivalent antibodies, such as IgG-scFv antibodies (for example, wherein a first binding domain is an intact IgG and a second binding domain is an scFv attached to the first binding domain at the N-terminus of a light chain and/or at the C-terminus of a light chain and/or at the N-terminus of a heavy chain and/or at the C-terminus of a heavy chain of the IgG, or vice versa) including but not limited to bivalent antibodies with binding specificity for the first epitope within a polypeptide consisting of the amino acid sequence of TDVLNQSKPVTL (SEQ ID NO: 177) and with binding specificity for the second epitope within extracellular domain 3 (ECD3) of a US28 protein of HCMV, for example wherein the first and/or second epitope are as further defined by the present application;
(b) monovalent antibodies, such as a DuoBody® or ‘knob-in-hole’ bispecific antibody (for example, an scFv-KIH, scFv-KIH^{r}, a BiTE-KIH or a BiTE-KIH^{r};
(c) scFv₂-Fc antibodies;
(d) bispecific antibodies, such as bispecific T-cell engager (BiTE) antibodies;
(e) dual variable domain (DVD)-Ig antibodies;
(f) dual-affinity re-targeting (DART)-based antibodies (for example, DART₂-Fc or DART);
(g) trispecific antibodies, such as DNL-Fab₃ antibodies or trispecific antibodies with binding specificity for the first epitope within a polypeptide consisting of the amino acid sequence of TDVLNQSKPVTL (SEQ ID NO: 177) and with binding specificity for the second epitope within extracellular domain 3 (ECD3) of a US28 protein of HCMV, for example wherein the first and/or second epitope are as defined by the present application, and binding specificity for a third epitope such as an epitope presented by an immune cell, optionally wherein the trispecific antibody is a trispecific immune cell engager antibody, for example, a trispecific T-cell engager (TiTE), and optionally wherein the TiTE antibody comprises a CD3-binding domain;
(h) scFv-HSA-scFv antibodies;
(i) single domain antibodies;
(j) heavy-chain-only IgGs (hcIgGs), such as camelid IgG (e.g. VHH antibodies) and shark immunoglobulin new antigen receptor (IgNAR), and single chain antibodies thereof; and
(k) a chimeric antigen receptor (CAR) comprising an extracellular domain that composes, consists essentially of, or consists of a binding molecule according to the first aspect of the present invention, for example an extracellular domain that comprises any one of options (a) to (h) of this list, or combinations thereof.

Additionally, or alternatively, a binding molecule according to the first aspect of the present invention may be selected from the group consisting of:
(i) a bispecific immune cell engager antibody, for example, a bispecific T-cell engager (BITE), and optionally wherein the BiTE antibody comprises a CD3-binding domain; or
(ii) monoclonal antibody, optionally a recombinant monoclonal antibody, for example, a monoclonal antibody produced recombinantly by CHO cells.

The first aspect of the present invention also provides a functional fragment of a binding molecule as defined above, wherein the functional fragment:
(a) comprises or consists of an antigen-binding fragment of a binding molecule as defined by any of the preceding claims, or a variant, fusion or derivative thereof selected from the group consisting of: an Fv fragment (such as a single chain Fv fragment (scFv), or a disulphide-bonded Fv fragment), a Fab-like fragment (such as a Fab fragment, a Fab′ fragment or a F(ab)₂ fragment), and single domain antibodies (dAbs, including single and dual formats, such as dAb-linker-dAb and nanobodies);
(b) provides one or more of the binding characteristics of a binding molecule of the first aspect of the present invention, as defined herein;
(c) comprises the CDR sequences of a binding molecule of the first aspect of the present invention, as defined herein; and/or
(d) comprises the V_{H} and/or V_{L} sequences of a binding molecule of the first aspect of the present invention, as defined herein.

Fusions of the binding molecules of the first aspect of the present invention are also provided herein. For example, a binding molecule as defined above, or a functional fragment of said binding molecule as defined above, is provided wherein the binding molecule or the functional fragment thereof comprises a fusion polypeptide sequence, said fusion polypeptide sequence comprising a first amino acid sequence fused to a second amino acid sequence, wherein the first amino acid sequence comprises or consists of at least one of the polypeptide chains of the binding molecule or of the functional fragment thereof, and the second amino acid sequence is a fusion partner.

In certain embodiments, the binding molecule of the first aspect of the present invention is, or comprised within, a chimeric antigen receptor (CAR). Accordingly, the first aspect of the present invention also provides a CAR comprising:
(i) an extracellular domain, wherein the extracellular domain comprises or consists of a binding molecule of the first aspect of the present invention as defined herein, or a functional fragment of said binding molecule as defined herein;
(ii) a transmembrane domain; and
(iii) an intracellular domain;
wherein the extracellular domain of the CAR has binding specificity to a first epitope within a polypeptide consisting of the amino acid sequence of TDVLNQSKPVTL (SEQ ID NO: 177) within ECD1, and optionally also has binding specificity to a second epitope within ECD3, of a US28 protein of human cytomegalovirus (HCMV), and
wherein ECD1, SEQ ID NO: 177, and ECD3 of the US28 protein each comprise an amino acid sequence presented in the US28 protein at positions corresponding to positions 1 to 37, 26 to 37, and 167 to 183, respectively, of the US28 protein encoded by human cytomegalovirus (HCMV) as set forth in SEQ ID NO: 5.

Optionally, in said CAR:
(a) the extracellular domain of the CAR has binding specificity to a first epitope present entirely within a polypeptide consisting of the amino acid sequence of TDVLNQSKPVTL (SEQ ID NO: 177) within ECD1, and/or has binding specificity to a second epitope present entirely within ECD3, of the US28 protein of HCMV;
(b) the extracellular domain of the CAR has binding specificity to a first linear epitope within a polypeptide consisting of the amino acid sequence of TDVLNQSKPVTL (SEQ ID NO: 177) within ECD1, and/or has binding specificity to a second linear epitope within ECD3, of the US28 protein;
(c) the extracellular domain of the CAR has binding specificity to a first epitope within a polypeptide consisting of the amino acid sequence of TDVLNQSKPVTL (SEQ ID NO: 177) within ECD1 of a US28 protein of HCMV that is HCMV strain agnostic, for example, binding specificity to an epitope within ECD1 of a US28 protein of HCMV that is agnostic to two or more (such as all) of HCMV strains selected from the group consisting of DB, Towne, AD169, DAVIS, BL, JP, Merlin, PH, TB40/E, Toledo, TR, VHL/E and VR1814 (FIX), and/or has binding specificity to a second epitope within ECD3 of a US28 protein of HCMV that is HCMV strain agnostic, for example, binding specificity to an epitope within ECD3 of a US28 protein of HCMV that is agnostic to two or more (such as all) of HCMV strains selected from the group consisting of DB, Towne, AD169, DAVIS, BL, JP, Merlin, PH, TB40/E, Toledo, TR, VHL/E and VR181.4 (FIX);
(d) the extracellular domain of the CAR has specificity to a first epitope within a polypeptide consisting of the amino acid sequence of TDVLNQSKPVTL (SEQ ID NO: 177) within ECD1 of the US28 protein of multiple strains of HCMV, irrespective of whether the multiple HCMV strains show inter-strain sequence variation in one or more positions in the region of positions 1 to 25 of ECD1 (such as any one or more of positions 8, 15, 18, 19, 24, 25, including but not limited to the sequence variations listed in Table 3 of the present application), for example wherein the multiple strains are two, three, four, five, six, seven, eight, nine or ten different strains selected from the group consisting of DB, Toledo, Towne, VR1814, TB40/E, Merlin, AD169, VHL/E, Davis and BL; and/or
(e) the extracellular domain of the CAR has specificity to a second epitope within ECD3 of the US28 protein of HCMV, irrespective of whether the ECD3 of the US28 protein comprises the sequence of:
TKKDNQCMTDYDYLEVS (SEQ ID NO: 7) as found in ECD3 of US28 as encoded by a majority of HCMV strains, or
TKKNNQCMTDYDYLEVS (SEQ ID NO: 6) as found in ECD3 of US28 as encoded by a minority of HCMV strains.

In certain embodiments, in a CAR according to the first aspect of the present invention:
(a) the extracellular domain of the CAR has binding specificity to the first epitope within a polypeptide consisting of the amino acid sequence of SEQ ID NO: 177, and comprises one, two, three, four, five or six complementarity determining regions (CDRs) corresponding to any one, two, three, four, five or all six of the CDR sequences of antibody 4H3C3 as defined by SEQ ID NOs: 196, 197, 198, 199, 200 and 201, respectively, and/or a functional variant (such as a 4H3C3 humanised variant) of any one or more of said CDR sequences of antibody 4H3C3;
(b) the extracellular domain of the CAR has binding specificity to the first epitope within a polypeptide consisting of the amino acid sequence of SEQ ID NO: 177, and comprises:
(i) one, two, or all three, of the CDR 1, 2, and 3, sequences of the variable heavy chain (V_{H}) of antibody 4H3C3, as defined by SEQ ID NOs: 196, 197, and 198, respectively, or as defined by a 4H3C3 humanised variant thereof; and/or
(ii) one, two, or all three, of the CDR 1, 2, and 3, sequences of the variable light chain (V_{L}) of antibody 4H3C3, as defined by SEQ ID NOs: 199, 200, and 201, respectively, or as defined by a 4H3C3 humanised variant thereof; and/or
(c) the extracellular domain of the CAR has binding specificity to the first epitope within a polypeptide consisting of the amino acid sequence of SEQ ID NO: 177, and comprises:
(i) at least one variable heavy chain (V_{H}) polypeptide sequence that comprises CDR 1, 2, and 3 sequences having the sequences of SEQ ID Nos: 196, 197, and 198, respectively (or a 4H3C3 humanised variant thereof), and optionally wherein the at least one variable heavy chain (V_{H}) polypeptide sequence comprises, consists essentially of, or consists of, the sequence of SEQ ID NO: 187 (or a 4H3C3 humanised variant thereof); and/or
(ii) at least one variable light chain (V_{L}) polypeptide sequence that comprises CDR 1, 2 and 3 sequences having the sequences of SEQ ID NOs: 199, 200, and 201, respectively (or a 4H3C3 humanised variant thereof), and optionally wherein the variable light chain (V_{L}) polypeptide comprises, consists essentially of, or consists of, the sequence of SEQ ID NO: 189 (or a 4H3C3 humanised variant thereof).

In another embodiment, in a CAR according to the first aspect of the present invention:
(a) the extracellular domain of the CAR has binding specificity to the first epitope within a polypeptide consisting of the amino acid sequence of SEQ ID NO: 177, and comprises one, two, three, four, five or six complementarity determining regions (CDRs) corresponding to any one, two, three, four, five or all six of the CDR sequences of antibody 7B1F3 as defined by SEQ ID NOs: 219, 220, 221, 199, 222 and 223, respectively, and/or a functional variant (such as a 7B1F3 humanised variant) of any one or more of said CDR sequences of antibody 7B1F3;
(b) the extracellular domain of the CAR has binding specificity to the first epitope within a polypeptide consisting of the amino acid sequence of SEQ ID NO: 177, and comprises:
(i) one, two, or all three, of the CDR 1, 2, and 3, sequences of the variable heavy chain (V_{H}) of antibody 7B1F3, as defined by SEQ ID NOs: 219, 220, and 221, respectively, or as defined by a 7B1F3 humanised variant thereof; and/or
(ii) one, two, or all three, of the CDR 1, 2, and 3, sequences of the variable light chain (V_{L}) of antibody 7B1F3, as defined by SEQ ID NOs: 199, 222, and 223, respectively, or as defined by a 7B1F3 humanised variant thereof; and/or
(c) the extracellular domain of the CAR has binding specificity to the first epitope within a polypeptide consisting of the amino acid sequence of SEQ ID NO: 177, and comprises:
(i) at least one variable heavy chain (V_{H}) polypeptide sequence that comprises CDR 1, 2, and 3 sequences having the sequences of SEQ ID Nos: 219, 220, and 221, respectively (or a 7B1F3 humanised variant thereof), and optionally wherein the at least one variable heavy chain (V_{H}) polypeptide sequence comprises, consists essentially of, or consists of, the sequence of SEQ ID NO: 211 (or a 7B1F3 humanised variant thereof); and/or
(ii) at least one variable light chain (V_{L}) polypeptide sequence that comprises CDR 1, 2 and 3 sequences having the sequences of SEQ ID NOs: 199, 222, and 223, respectively (or a 7B1F3 humanised variant thereof), and optionally wherein the variable light chain (V_{L}) polypeptide comprises, consists essentially of, or consists of, the sequence of SEQ ID NO: 213 (or a 7B1F3 humanised variant thereof).

In another embodiment, in a CAR according to the first aspect of the present invention:
(a) the extracellular domain of the CAR has binding specificity to the first epitope within a polypeptide consisting of the amino acid sequence of SEQ ID NO: 177, and comprises one, two, three, four, five or six complementarity determining regions (CDRs) corresponding to any one, two, three, four, five or all six of the CDR sequences of antibody 2F5B11 as defined by SEQ ID NOs: 242, 243, 244, 199, 245 and 246, respectively, and/or a functional variant of any one or more of said CDR sequences of antibody 2F5B11;
(b) the extracellular domain of the CAR has binding specificity to the first epitope within a polypeptide consisting of the amino acid sequence of SEQ ID NO: 177, and comprises:
(i) one, two, or all three, of the CDR 1, 2, and 3, sequences of the variable heavy chain (V_{H}) of antibody 2F5B11, as defined by SEQ ID NOs: 242, 243, and 244, respectively; and/or
(ii) one, two, or all three, of the CDR 1, 2, and 3, sequences of the variable light chain (V_{L}) of antibody 2F5B11, as defined by SEQ ID NOs: 199, 245, and 246, respectively; and/or
(c) the extracellular domain of the CAR has binding specificity to the first epitope within a polypeptide consisting of the amino acid sequence of SEQ ID NO: 177, and comprises:
(i) at least one variable heavy chain (V_{H}) polypeptide sequence that comprises CDR 1, 2, and 3 sequences having the sequences of SEQ ID Nos: 242, 243, and 244, respectively, and optionally wherein the at least one variable heavy chain (V_{H}) polypeptide sequence comprises, consists essentially of, or consists of, the sequence of SEQ ID NO: 233; and/or
(ii) at least one variable light chain (V_{L}) polypeptide sequence that comprises CDR 1, 2 and 3 sequences having the sequences of SEQ ID NOs: 199, 245, and 246, respectively, and optionally wherein the variable light chain (V_{L}) polypeptide comprises, consists essentially of, or consists of, the sequence of SEQ ID NO: 235.

In certain options, in a CAR according to the first aspect of the present invention:
(a) the extracellular domain of the CAR further has binding specificity to the second epitope within ECD3 of a US28 protein of HCMV, and comprises one, two, three, four, five or six complementarity determining regions (CDRs) corresponding to any one, two, three, four, five or all six of the CDR sequences of antibody 1D3 as defined by SEQ ID NOs: 8, 9, 10, 14, 15 and 16, respectively, and/or a functional variant of any one or more of said CDR sequences of antibody 1D3;
(b) the extracellular domain of the CAR further has binding specificity to the second epitope within ECD3 of a US28 protein of HCMV, and comprises:
(i) one, two, or all three, of the CDR 1, 2, and 3, sequences of the variable heavy chain (V_{H}) of antibody 1D3, as defined by SEQ ID NOs: 8, 9, and 10, respectively; and/or
(ii) one, two, or all three, of the CDR 1, 2, and 3, sequences of the variable light chain (V_{L}) of antibody 1D3, as defined by SEQ ID NOs: 14, 15, and 16, respectively; and/or
(c) the extracellular domain of the CAR further has binding specificity to the second epitope within ECD3 of a US28 protein of HCMV, and comprises: (i) at least one variable heavy chain (V_{H}) polypeptide sequence that comprises CDR 1, 2, and 3 sequences having the sequences of SEQ ID Nos: 8, 9, and 10, respectively, and optionally wherein the at least one variable heavy chain (V_{H}) polypeptide sequence comprises, consists essentially of, or consists of, the sequence of SEQ ID NO: 12; and/or (ii) at least one variable light chain (V_{L}) polypeptide sequence that comprises CDR 1, 2 and 3 sequences having the sequences of SEQ ID NOs: 14, 15, and 16, respectively, and optionally wherein the variable light chain (V_{L}) polypeptide comprises, consists essentially of, or consists of, the sequence of SEQ ID NO: 18.

In certain other options, in a CAR according to the first aspect of the present invention:
(a) the extracellular domain of the CAR further has binding specificity to the second epitope within ECD3 of a US28 protein of HCMV, and comprises one, two, three, four, five or six complementarity determining regions (CDRs) corresponding to any one, two, three, four, five or all six of the CDR sequences of antibody 1C10 as defined by SEQ ID NOs: 112, 113, 114, 117, 83 and 118, respectively, and/or a functional variant of any one or more of said CDR sequences of antibody 1C10;
(b) the extracellular domain of the CAR further has binding specificity to the second epitope within ECD3 of a US28 protein of HCMV, and comprises:
(i) one, two, or all three, of the CDR 1, 2, and 3, sequences of the variable heavy chain (V_{H}) of antibody 1C10, as defined by SEQ ID NOs: 112, 113, and 114, respectively; and/or
(ii) one, two, or all three, of the CDR 1, 2, and 3, sequences of the variable light chain (V_{L}) of antibody 1C10, as defined by SEQ ID NOs: 117, 83, and 118, respectively; and/or
(c) the extracellular domain of the CAR further has binding specificity to the second epitope within ECD3 of a US28 protein of HCMV, and comprises:
(i) at least one variable heavy chain (V_{H}) polypeptide sequence that comprises CDR 1, 2, and 3 sequences having the sequences of SEQ ID Nos: 112, 113, and 114, respectively, and optionally wherein the at least one variable heavy chain (V_{H}) polypeptide sequence comprises, consists essentially of, or consists of, the sequence of SEQ ID NO: 104; and/or
(ii) at least one variable light chain (V_{L}) polypeptide sequence that comprises CDR 1, 2 and 3 sequences having the sequences of SEQ ID NOs: 117, 83, and 118, respectively, and optionally wherein the variable light chain (V_{L}) polypeptide comprises, consists essentially of, or consists of, the sequence of SEQ ID NO: 108.

In certain other options, in a CAR according to the first aspect of the present invention:
(a) the extracellular domain of the CAR further has binding specificity to the second epitope within ECD3 of a US28 protein of HCMV, and comprises one, two, three, four, five or six complementarity determining regions (CDRs) corresponding to any one, two, three, four, five or all six of the CDR sequences of antibody 1A10 as defined by SEQ ID NOs: 112, 113, 114, 117, 83 and 118, respectively, and/or a functional variant of any one or more of said CDR sequences of antibody 1A10;
(b) the extracellular domain of the CAR further has binding specificity to the second epitope within ECD3 of a US28 protein of HCMV, and comprises:
(i) one, two, or all three, of the CDR 1, 2, and 3, sequences of the variable heavy chain (V_{H}) of antibody 1A10, as defined by SEQ ID NOs: 112, 113, and 114, respectively; and/or
(ii) one, two, or all three, of the CDR 1, 2, and 3, sequences of the variable light chain (V_{L}) of antibody 1A10, as defined by SEQ ID NOs: 117, 83, and 118, respectively; and/or
(c) the extracellular domain of the CAR further has binding specificity to the second epitope within ECD3 of a US28 protein of HCMV, and comprises:
(i) at least one variable heavy chain (V_{H}) polypeptide sequence that comprises CDR 1, 2, and 3 sequences having the sequences of SEQ ID Nos: 112, 113, and 114, respectively, and optionally wherein the at least one variable heavy chain (V_{H}) polypeptide sequence comprises, consists essentially of, or consists of, the sequence of SEQ ID NO: 122; and/or
(ii) at least one variable light chain (V_{L}) polypeptide sequence that comprises CDR 1, 2 and 3 sequences having the sequences of SEQ ID NOs: 117, 83, and 118, respectively, and optionally wherein the variable light chain (V_{L}) polypeptide comprises, consists essentially of, or consists of, the sequence of SEQ ID NO: 126.

In certain other options, in a CAR according to the first aspect of the present invention:
(a) the extracellular domain of the CAR further has binding specificity to the second epitope within ECD3 of a US28 protein of HCMV, and comprises one, two, three, four, five or six complementarity determining regions (CDRs) corresponding to any one, two, three, four, five or all six of the CDR sequences of antibody 1G9 as defined by SEQ ID NOs: 76, 77, 78, 82, 83 and 84, respectively, and/or a functional variant of any one or more of said CDR sequences of antibody 1G9;
(b) the extracellular domain of the CAR further has binding specificity to the second epitope within ECD3 of a US28 protein of HCMV, and comprises:
(i) one, two, or all three, of the CDR 1, 2, and 3, sequences of the variable heavy chain (V_{H}) of antibody 1G9, as defined by SEQ ID NOs: 76, 77, and 78, respectively; and/or
(ii) one, two, or all three, of the CDR 1, 2, and 3, sequences of the variable light chain (V_{L}) of antibody 1G9, as defined by SEQ ID NOs: 82, 83, and 84, respectively; and/or
(c) the extracellular domain of the CAR further has binding specificity to the second epitope within ECD3 of a US28 protein of HCMV, and comprises:
(i) at least one variable heavy chain (V_{H}) polypeptide sequence that comprises CDR 1, 2, and 3 sequences having the sequences of SEQ ID Nos: 76, 77, and 78, respectively, and optionally wherein the at least one variable heavy chain (VW) polypeptide sequence comprises, consists essentially of, or consists of, the sequence of SEQ ID NO: 68; and/or
(ii) at least one variable light chain (V_{L}) polypeptide sequence that comprises CDR 1, 2 and 3 sequences having the sequences of SEQ ID NOs: 82, 83, and 84, respectively, and optionally wherein the variable light chain (V_{L}) polypeptide comprises, consists essentially of, or consists of, the sequence of SEQ ID NO: 72.

In certain other options, in a CAR according to the first aspect of the present invention:
(a) the extracellular domain of the CAR further has binding specificity to the second epitope within ECD3 of a US28 protein of HCMV, and comprises one, two, three, four, five or six complementarity determining regions (CDRs) corresponding to any one, two, three, four, five or all six of the CDR sequences of antibody 1E8 as defined by SEQ ID NOs: 76, 95, 96, 82, 99 and 100, respectively, and/or a functional variant of any one or more of said CDR sequences of antibody 1E8;
(b) the extracellular domain of the CAR further has binding specificity to the second epitope within ECD3 of a US28 protein of HCMV, and comprises:
(i) one, two, or all three, of the CDR 1, 2, and 3, sequences of the variable heavy chain (V_{H}) of antibody 1E8, as defined by SEQ ID NOs: 76, 95, and 96, respectively; and/or
(ii) one, two, or all three, of the CDR 1, 2, and 3, sequences of the variable light chain (V_{L}) of antibody 1E8, as defined by SEQ ID NOs: 82, 99, and 100, respectively; and/or
(c) the extracellular domain of the CAR further has binding specificity to the second epitope within ECD3 of a US28 protein of HCMV, and comprises:
(i) at least one variable heavy chain (V_{H}) polypeptide sequence that comprises CDR 1, 2, and 3 sequences having the sequences of SEQ ID Nos: 76, 95, and 96, respectively, and optionally wherein the at least one variable heavy chain (V_{H}) polypeptide sequence comprises, consists essentially of, or consists of, the sequence of SEQ ID NO: 88; and/or
(ii) at least one variable light chain (V_{L}) polypeptide sequence that comprises CDR 1, 2 and 3 sequences having the sequences of SEQ ID NOs: 82, 99, and 100, respectively, and optionally wherein the variable light chain (V_{L}) polypeptide comprises, consists essentially of, or consists of, the sequence of SEQ ID NO: 92.

Optionally, in said CAR, the extracellular domain is an antibody, for example a single-chain variable fragment (scFv).

The transmembrane domain of a CAR according to the first aspect of the present invention may, for example, comprise the transmembrane domain of a protein, for example the transmembrane domain of a transmembrane receptor protein, and optionally wherein the transmembrane domain comprises the transmembrane domain of a protein selected from the group consisting of the alpha, beta or zeta chain of the T-cell receptor, CD28, CD3 epsilon, CD8, CD45 and CD4.

The extracellular domain of a CAR according to the first aspect of the present invention may, for example, be connected to the transmembrane domain by a hinge region.

The intracellular domain of a CAR according to the first aspect of the present invention may, for example, comprise an intracellular signalling domain, for example wherein: (a) the intracellular signalling domain comprises one or more immunoreceptor tyrosine-based activation motifs (ITAMs); and/or (b) the intracellular signalling domain comprises a signalling domain of CD3 zeta, Fc receptor gamma, Fc receptor beta, CD3 gamma, CD3 delta, CD3 epsilon, CD5, CD22, CD79a, CD79b, and CD66d.

The intracellular domain of a CAR according to the first aspect of the present invention may, for example, comprise one or more costimulatory domains, for example: (a) wherein the one or more costimulatory domains includes one or more functional signalling domains obtained from a protein selected from the group consisting of CD28, 41BB, OX40, ICOS, CD27, and DAP10; (b) wherein the intracellular domain incorporates a costimulatory domain proximal to the intracellular signalling domain, (c) wherein the intracellular domain comprises two or more costimulatory domains, for example two in-line costimulatory domains, and/or (d) wherein the intracellular domain incorporates separate cytokine signals.

A CAR according to the first aspect of the present invention may, for example, additionally comprise a leader sequence.

A second aspect of the present invention provides a nucleic acid molecule, or combination of multiple distinct nucleic acid molecules, wherein the nucleic acid molecule comprises, or the combination of multiple distinct nucleic acid molecules collectively comprise, one or more nucleic acid sequences that, individually or in combination, encode the binding molecule of the first aspect of the present invention, for example an antibody or CAR according to the first aspect of the present invention.

A third aspect of the present invention provides a vector comprising (or combination of multiple distinct vectors which collectively comprise) a nucleic acid molecule according to the second aspect of the present invention, or combination of multiple distinct nucleic acid molecules according to the second aspect of the present invention. The, or each, vector of the third aspect of the present invention may, for example, be selected from the group consisting of a retroviral vector, a plasmid, a lentivirus vector, and an adenoviral vector.

Optionally a nucleic acid molecule, or combination of multiple distinct nucleic acid molecules according to the second aspect of the present invention, and/or a vector according to the third aspect of the present invention, comprise one or more additional sequences, wherein the or each additional sequence encodes one or more selectable markers.

A fourth aspect of the present invention provides a cell, or a population of cells (optionally a homogeneous or heterogeneous population of cells) comprising the nucleic acid molecule, or combination of multiple distinct nucleic acid molecules, according to the second aspect of the present invention, and/or a vector according to the third aspect of the present invention, optionally wherein the cell expresses one or more binding molecules according to the first aspect of the present invention (such as one or more antibodies, and/or one or more CARs), said one or more binding molecules and/or CARs being encoded by the nucleic acid molecule, or combination of multiple distinct nucleic acid molecules, according to the second aspect of the present invention, or a vector according to the third aspect of the present invention. Said cells may, optionally, be selected from isolated cells, ex vivo cells, and in vitro cells.

A cell according to the fourth aspect of the present invention may, for example, comprise: (a) a nucleic acid molecule, or combination of multiple distinct nucleic acid molecules, according to the second aspect of the present invention, wherein the encoded binding molecule is an antibody, a functional fragment of said antibody, or an antibody of functional fragment thereof that comprises a fusion polypeptide sequence, according to the first aspect of the present invention; and/or (b) a vector according to the third aspect of the present invention, wherein said vector comprises a nucleic acid molecule, or combination of multiple distinct nucleic acid molecules as defined by option (a) of this paragraph.

A cell according to the fourth aspect of the present invention may, for example, comprise: (a) a nucleic acid molecule, or combination of multiple distinct nucleic acid molecules, according to the second aspect of the present invention, wherein the encoded binding molecule is a CAR according to the first aspect of the present invention; and/or (b) a vector according to the third aspect of the present invention, wherein said vector comprises a nucleic acid molecule, or combination of multiple distinct nucleic acid molecules as defined by part (a) of this paragraph. Without limitation, said cell may, for example, be selected from the group consisting of: a T cell, natural killer (NK) cell, and a macrophage. Accordingly, the cell may optionally be a CAR-T cell, a CAR-NK cell or a CAR-macrophage, and optionally, when the cell is a CAR-T cell, then for example the T-cell may be selected from the group consisting of CD8⁺ T cells, CD4+ T cells, effector T cells, helper T cells, memory T cells, cytotoxic T lymphocytes (CTLs), EBV-specific T cell receptor (TCR) or γδ-T cell subtypes.

The fourth aspect of the present invention also provides a cell comprising a binding molecule according the first aspect of the present invention and/or a nucleic acid encoding said binding molecule, optionally wherein said nucleic acid is a nucleic acid or vector as defined by the second or third aspects of the present invention, respectively. For example, the binding molecule may be an antibody according the first aspect of the present invention, a functional fragment of said antibody according the first aspect of the present invention, or an antibody of functional fragment thereof that comprises a fusion polypeptide sequence according the first aspect of the present invention, and optionally wherein the antibody is monoclonal antibody, and further for example wherein the cell is a mammalian cell, such as a CHO cell, that recombinantly expresses the monoclonal antibody.

The fourth aspect of the present invention also provides a cell comprising a CAR according to the first aspect of the present invention and/or a nucleic acid encoding said CAR, optionally wherein said nucleic acid is a nucleic acid or vector as defined by the second or third aspects of the present invention, respectively. Without limitation, said cell may, for example, be selected from the group consisting of: a T cell, natural killer (NK) cell, and a macrophage. Accordingly, the cell may optionally be a CAR-T cell, a CAR-NK cell or a CAR-macrophage, and optionally, when the cell is a CAR-T cell, then for example the T-cell may be selected from the group consisting of CD8⁺ T cells, CD4+ T cells, effector T cells, helper T cells, memory T cells, cytotoxic T lymphocytes (CTLs), EBV-specific T cell receptor (TCR) or γδ-T cell subtypes.

A fifth aspect of the present invention provides a method of producing a cell, more particularly a recombinant cell, or a population of such cells (optionally a homogeneous or heterogeneous population of cells), the method comprising introducing a nucleic acid molecule, or combination of multiple distinct nucleic acid molecules, according to the second aspect of the present invention, and/or a vector according to third aspect of the present invention, into a cell.

Said method optionally further comprises a step of selecting cells according to the fifth aspect of the present invention; for example selecting said cells from a heterogeneous cell population, thereby to create an enriched and/or homogeneous cell population. Said selection step may include selecting for the presence of one or more selectable markers present in the nucleic acid molecule, or combination of multiple distinct nucleic acid molecules, according to the second aspect of the present invention, and/or a vector according to third aspect of the present invention.

A sixth aspect of the present invention provides a method of producing a binding molecule according to the first aspect of the present invention, for example an antibody or a CAR according to the first aspect of the present invention, the method comprising: expressing a nucleic acid molecule, or combination of multiple distinct nucleic acid molecules, according to the second aspect of the present invention, and/or a vector according to the third aspect of the present invention, in a cell, more particularly a recombinant cell, or a population of such cells (optionally a homogeneous or heterogeneous population of cells). Optionally, the method of the sixth aspect of the present invention may also comprise the step of isolating the thus-produced binding molecule from the cell; for example, wherein the binding molecule is an antibody according to the first aspect of the present invention, a functional fragment of said antibody, or an antibody of functional fragment thereof that comprises a fusion polypeptide sequence according to the first aspect of the present invention. Said cells may, optionally, be selected from isolated cells, ex vivo cells, and in vitro cells. A seventh aspect of the present invention provides an isolated binding molecule that is obtained, or obtainable, by the method of the sixth aspect of the present invention, optionally, wherein the isolated binding molecule is further formulated for administration to a subject.

An eighth aspect of the present invention provides a conjugate, the conjugate comprising a moiety conjugated to a binding molecule as defined by the first aspect of the present invention, or to a functional fragment of said binding molecule. Without limitation, said moiety may for example be a therapeutic, prophylactic, diagnostic, prognostic, or theragnostic moiety. In some embodiments, the moiety is a drug (for example, wherein the conjugate is an antibody-drug conjugate (“ADC”)) and/or a radioactive moiety (for example, wherein the conjugate is suitable for use in radioimmunotherapy (“RIT”)).

A ninth aspect of the present invention provides a method of producing a conjugate according to the eighth aspect of the present invention, the method comprising the steps of:
(a) providing a binding molecule as defined by the first aspect of the present invention, or a functional fragment of said binding molecule; and
(b) conjugating a moiety to the binding molecule, or to the functional fragment of said binding molecule.

The method of the ninth aspect of the present invention may additionally comprise the step of isolating the thus-produced conjugate. The isolated conjugate may therefore be presented in an isolated form, for example in the form of a composition wherein at least 50%, 60%, 70%, 80%, 90%, 95%, 96%, 97%, 98%, 99%, 99.5%, 99.9% or substantially 100% (by molar ratio) of the binding molecule, or the functional fragment of said binding molecule, is present in the form of the conjugate. Additionally or alternatively, the isolated form of the conjugate may be a composition wherein at least 50%, 60%, 70%, 80%, 90%, 95%, 96%, 97%, 98%, 99%, 99.5%, 99.9% or substantially 100% (by molar ratio) of the moiety, is present in the form of the conjugate. The ninth aspect of the present invention further provides the isolated conjugate, or a composition comprising said isolated conjugate.

In certain preferred embodiments of the conjugate of the eighth aspect of the present invention, or of the method of the ninth aspect of the present invention, the binding molecule is an antibody according to the first aspect of the present invention, a functional fragment of said antibody, or an antibody of functional fragment thereof that comprises a fusion polypeptide sequence according to the first aspect of the present invention.

A tenth aspect of the present invention provides an isolated conjugate that is obtained, or obtainable, by the method of the ninth aspect of the present invention, optionally, wherein the isolated conjugate is further formulated for administration to a subject.

An eleventh aspect of the present invention provides a method of combating HCMV or a disease or condition associated with HCMV, the method comprising administering to a subject, or to ex vivo or in vitro cellular material, any one or more agents selected from the group consisting of:
i. a binding molecule according to the first aspect of the present invention,
ii. a functional fragment of said binding molecule according to the first aspect of the present invention,
iii. an isolated binding molecule according to the seventh aspect of the present invention,
iv. a nucleic acid molecule, or combination of multiple distinct nucleic acid molecules, according to the second aspect of the present invention,
v. a vector according to the third aspect of the present invention,
vi. a cell according to the fourth aspect of the present invention,
vii. a conjugate according to the eighth aspect of the present invention, and
viii. an isolated conjugate according to the tenth aspect of the present invention.

To put it another way, the eleventh aspect of the present invention provides one or more of said agents for use in combating a disease or condition associated with HCMV in a subject, or in ex vivo or in vitro cellular material.

Further, the eleventh aspect of the present invention provides for the use one or more of said agents in the manufacture of a medicament for combating a disease or condition associated with HCMV in a subject, or in ex vivo or in vitro cellular material.

The disease or condition associated with HCMV, to be combatted in accordance with the eleventh aspect of the present invention may be an HCMV infection or be associated with an HCMV infection. The HCMV infection may, in one embodiment, be a single strain infection. Optionally, in an alternative embodiment, the HCMV infection comprises a multi-strain HCMV infection, wherein the multi-strain HCMV infection comprises infection with more than one different strain of HCMV, for example two or more HCMV strains that encode different US28 protein sequences. Said two or more strains may encode US28 proteins that differ in one or more of the extracellular regions, such as in the N-terminal (ECD1) region, the first extracellular loop (ECD2) region, the second extracellular loop (ECD3) region, and/or the third extracellular loop (ECD4) region.

In one embodiment of interest, the two or more HCMV strains in a multi-strain HCMV infection each encode a US28 protein that differs from the other at least in one or more positions of the N-terminal (ECD1) region; for example they may differ at 1, 2, 3, 4, 5, 6, 8, 9, 10 or more amino acid positions in the N-terminal (ECD1) region. For example, the two or more HCMV strains in a multi-strain HCMV infection may show inter-strain sequence variation in one or more positions in the region of positions 1 to 25 of ECD1 (such as any one or more of positions 8, 15, 18, 19, 24, 25, including but not limited to the sequence variations listed in Table 3 of the present application), for example wherein the multiple strains comprise at least two, three, four, five, six, seven, eight, nine or ten different strains selected from the group consisting of DB, Toledo, Towne, VR1814, TB40/E, Merlin, AD169, VHL/E, Davis and BL.

Additionally, or alternatively, in another embodiment of interest, the two or more HCMV strains in a multi-strain HCMV infection each encode a US28 protein that differs from the other at one or more positions of the second extracellular loop (ECD3) region, for example one or more of the HCMV strains in a multi-strain HCMV infection may encode a US28 protein that encodes the 4N-variant of ECD3, and one or more of the other HCMV strains in a multi-strain HCMV infection may encode a US28 protein that encodes the 4D-variant of ECD3.

The disease or condition associated with HCMV, to be combatted in accordance with the eleventh aspect of the present invention may be a latent HCMV infection (for example, a single or multi-strain latent HCMV infection) or be associated with a latent HCMV infection (optionally a multi-strain latent HCMV infection).

The disease or condition associated with HCMV, to be combatted in accordance with the eleventh aspect of the present invention may be a lytic HCMV infection (optionally a multi-strain lytic HCMV infection) or be associated with a lytic HCMV infection (optionally a multi-strain lytic HCMV infection).

The disease or condition associated with HCMV, to be combatted in accordance with the eleventh aspect of the present invention may be a congenital HCMV infection (for example, a single or multi-strain infection), such as a latent congenital single or multi-strain HCMV infection or a lytic congenital single or multi-strain HCMV infection;

The disease or condition associated with HCMV, to be combatted in accordance with the eleventh aspect of the present invention may be cancer, for example HCMV-infected cancer (optionally a single-strain, or multi-strain, HCMV infected cancer), such as latent HCMV-infected cancer (optionally a single-strain, or multi-strain, latent HCMV infected cancer).

The disease or condition associated with HCMV, to be combatted in accordance with the eleventh aspect of the present invention may be an epithelial cancer; optionally wherein the epithelial cancer is breast cancer; for example, wherein the breast cancer is triple negative breast cancer (TNBC), or a HER2-positive breast cancer. A HER2-positive breast cancer may, for example, be HER2+ HR− (wherein HR− means hormone receptor-negative, and refers to oestrogen receptor negative (ER−) and progesterone receptor negative (PR−) status); or HER2+ ER+ PR−; or HER2+ ER— PR+; or a triple positive form of breast cancer “TPBC” that is HER2+ ER+ PR+. It is noted that HER2+ HR− is a particularly aggressive form of breast cancer and is of high interest for diagnosis treatment in accordance with the present invention. Said forms of epithelial cancer may optionally be a single-strain, or multi-strain, form of HCMV infected epithelial cancer, for example a latent HCMV-infected form of epithelial cancer (optionally a single-strain, or multi-strain, latent HCMV infected cancer).

The disease or condition associated with HCMV, to be combatted in accordance with the eleventh aspect of the present invention, may be a metastasising and/or aggressive form of cancer. Said forms of metastasising and/or aggressive cancer may optionally be a single-strain, or multi-strain, form of HCMV infected metastasising and/or aggressive cancer, for example a latent HCMV-infected form of metastasising and/or aggressive cancer (optionally a single-strain, or multi-strain, latent HCMV infected cancer).

In some embodiments, the disease or condition associated with HCMV, to be combatted in accordance with the eleventh aspect of the present invention may be glioblastoma. In other embodiment described herein, the disease or condition is not glioblastoma and/or the subject to be treated does not have and/or has not been diagnosed as having glioblastoma.

In some embodiments, the subject (or the ex vivo or in vitro cellular material) to be treated in accordance with the eleventh aspect of the present invention may have, and/or have been diagnosed has having or possessing, HCMV-infected cancer cells, such as latent HCMV-infected cancer cells.

In some embodiments, the subject to be treated in accordance with the eleventh aspect of the present invention may be, or intended to be, the recipient of a cellular material, such as the donation of a cellular product. Said cellular product may, for example, comprise, consist essentially of, or consist of, living ex vivo cellular material selected from the group that includes: one or more types of ex vivo cells; one or more types of ex vivo cell cultures; one or more types of ex vivo tissues; one or more types of ex vivo tissue cultures; one or more types of ex vivo organs; and/or one or more types of ex vivo organ cultures. Optionally, the cellular product may be derived, directly or indirectly, from a living donor.

In some embodiments, the subject to be treated in accordance with the eleventh aspect of the present invention may be, or intended to be, the donor of a cellular material, such as the donor of a cellular product. Said cellular product may be comprise, consist essentially of, or consist of any one or more of cells, tissue or an organ from said donor.

In some embodiments, the ex vivo or in vitro cellular material to be treated in accordance with the eleventh aspect of the present invention may be an ex vivo cellular product. Said ex vivo cellular product may, for example, comprise, consist essentially of, or consist of, living ex vivo cellular material selected from the group that includes: one or more types of ex vivo cells; one or more types of ex vivo cell cultures; one or more types of ex vivo tissues; one or more types of ex vivo tissue cultures; one or more types of ex vivo organs; and/or one or more types of ex vivo organ cultures. Optionally, the cellular product may be derived, directly or indirectly, from a living donor.

In one embodiment, the one or more agents to be used in accordance with the eleventh aspect of the present invention may, for example, be (or include one or more agents) selected from the group consisting of:
i. a therapeutic antibody as defined by the first aspect of the present invention,
ii. a functional fragment of said therapeutic antibody as defined by the first aspect of the present invention,
iii. a therapeutic antibody that comprises a fusion polypeptide sequence as defined by the first aspect of the present invention; and
iv. a functional fragment of said therapeutic antibody that comprises a fusion polypeptide sequence as defined by the first aspect of the present invention.

For example, the one or more agents used in accordance with the eleventh aspect of the present invention may be (or include one or more agents selected from) a bispecific antibody as defined by the first aspect of the present invention. Without limitation, this may be a bispecific immune cell engager antibody. An exemplary embodiment thereof is a bispecific T-cell engager (BiTE) antibody, optionally wherein, in addition to the region that comprises the binding molecule of the first aspect of the present invention which has binding specificity for the ECD1 region of the US28 protein, the BiTE antibody further comprises a T-cell engaging domain, such as a CD3-binding domain.

In a further embodiment, the one or more agents used in accordance with the eleventh aspect of the present invention may be (or include one or more agents selected from) a trispecific antibody as defined by the first aspect of the present invention. An exemplary embodiment thereof is a trispecific antibody wherein, in addition to the region that comprises the binding molecule of the first aspect of the present invention which has binding specificity for the first epitope within a polypeptide consisting of the amino acid sequence of SEQ ID NO: 177 within ECD1 region of the US28 protein, the antibody further comprises a T-cell engaging domain, such as a CD3-binding domain, and an ECD3 engaging domain, such as the ECD3 binding molecules described herein.

In a further embodiment, the one or more agents to be used in accordance with the eleventh aspect of the present invention may, for example, be (or include) a conjugate according to the eighth aspect of the present invention and/or the tenth aspect of the present invention, such as conjugate that is an antibody-drug conjugate (“ADC”), or a conjugate that comprises radioactive moiety, such as conjugate that is suitable for use in radioimmunotherapy (“RIT”).

In some embodiments, the ADC comprises an anti-mitotic payload (e.g., tubulin inhibitors such as maytansines, etc). In some embodiments, the ADC comprises a payload that is a DNA damaging agent (e.g., topoisomerase inhibitors such as SN38, etc). In some embodiments, the ADC comprises a payload that is a transcription inhibitor (e.g., RNA Pol II inhibitors such as amanitin, etc). In some embodiments, the ADC comprises a payload that is selected from the group consisting of maytansinoids (such as maytansine, DM1, DM3 and DM4); auristatin payloads (such as Dolastatin 10, MMAE, MMAF and PF-06380101); tubulysins (such as tubulysin A, tubulysin B, tubulysin C, tubulysin G and tubulysin I); eribulin; taxol derivatives (such as doxetaxel and paclitaxel); DNA damaging agents (such as Calicheamicin; Anthramycin-based dimers, including pyrrolobenzodiazepine (PBD) dimers, indolinobenzodiazepine dimers (IGN), and pyrridinobenzodiazepines (PDD); Duocarrnycin-based payloads; Anthracyclines Doxorubicin, Ladirubicin, and PNU-159682; and Carnptothecin-based molecules, including Camptothecin, Exatecan and SN-38 (7-ethyl-10-hydroxycamptothecin)).

In another embodiment, the one or more agents to be used in accordance with the eleventh aspect of the present invention may, for example, be (or include) a cell (or population of cells, for example a homogeneous population of cells) wherein the or each cell comprises a CAR according to the fourth aspect of the present invention. Said cell or population of cells is typically isolated and/or formulated for administration to a subject. The or each cell may, for example, comprise a CAR according to the first aspect of the present invention and/or a nucleic acid encoding said CAR, optionally wherein said nucleic acid is a nucleic acid or vector as defined by the second or third aspects of the present invention, respectively. The or each cell may, for example, be a cell according to the fourth aspect of the present invention. Without limitation, said cell or cells may, for example, be selected from the group consisting of: a T cell, natural killer (NK) cell, and a macrophage. Accordingly, the cell may optionally be a CAR-T cell, a CAR-NK cell or a CAR-macrophage, and optionally, when the cell is a CAR-T cell, then for example the T-cell may be selected from the group consisting of CD8+ T cells, CD4+ T cells, effector T cells, helper T cells, memory T cells, cytotoxic T lymphocytes (CTLs), EBV-specific T cell receptor (TCR) or γδ-T cell subtypes.

Optionally, in accordance with the eleventh aspect of the present invention, the subject may be administered a further substance, such as a further therapeutic, prophylactic, diagnostic, prognostic, or theragnostic substance (such as an ECD3-binding molecule described herein), and optionally wherein the further substance may be administered separately, sequentially or simultaneously with the, or each of the one or more agents.

Accordingly, the eleventh aspect of the present invention also provides a method of treating a subject in need thereof, by administering to the subject a therapeutic, prophylactic, diagnostic, prognostic, or theragnostic substance, wherein the subject is also treated separately, sequentially (for example, before, or after), or simultaneously, with the, or each of the one or more agents.

In the embodiment in which the treatment is simultaneous, then the one or more agents to be used in accordance with the eleventh aspect of the present invention may be formulated and/or administered in combination with the therapeutic, prophylactic, diagnostic, prognostic, or theragnostic substance; or may be formulated separately but administered simultaneously as two separate formulations.

For example, in one embodiment, the disease or condition to be treated may be a form of cancer (such as one or more forms of cancer as disclosed above), and the additional therapeutic, prophylactic, diagnostic, prognostic, or theragnostic substance may be targeted to the cancer.

A twelfth aspect of the present invention provides an agent for use in medicine, wherein the agent is selected from the group consisting:
i. a binding molecule according to the first aspect of the present invention,
ii. a functional fragment of said binding molecule as defined by the first aspect of the present invention,
iii. an isolated binding molecule according to the seventh aspect of the present invention,
iv. a nucleic acid molecule, or combination of multiple distinct nucleic acid molecules, according to the second aspect of the present invention,
v. a vector according to the third aspect of the present invention,
vi. a cell according to the fourth aspect of the present invention,
vii. a conjugate according to the eighth aspect of the present invention, and
viii. an isolated conjugate according to the tenth aspect of the present invention.

Said agent may, for example, be an agent as define above, in respect of the eleventh aspect of the present invention.

A thirteenth aspect of the present invention provides a peptide or polypeptide comprising, consisting essentially of, or consisting of, the sequence TDVLNQSKPVTL (SEQ ID NO: 177), or comprising the sequence of an immunogenic fragment of SEQ ID NO: 177. Said peptide or polypeptide is not the US28 protein. Preferably the only US28-derived sequence in said peptide or polypeptide is the sequence of SEQ ID NO: 177 or the sequence of the immunogenic fragment of SEQ ID NO: 177 and/or said peptide or polypeptide does not contain any sequence of at 3, 4, 5, 6, 7, 8, 9, 10 or more than 10 contiguous amino acids that is identical to a sequence contiguous amino acid sequence of equivalent length found in the US28 protein at a location other than the region comprising the sequence of SEQ ID NO: 177.

A fourteenth aspect of the present invention provides a peptide or polypeptide comprising, consisting essentially of, or consisting of, the sequence TKKNNQCMTDYDYLEVS (SEQ ID NO: 6) and/or TKKDNQCMTDYDYLEVS (SEQ ID NO: 7), or comprising the sequence of an immunogenic fragment of SEQ ID NO: 6 and/or 7. Said peptide or polypeptide is not the US28 protein. Optionally said peptide or polypeptide also comprises the sequence of SEQ ID NO: 177 or the sequence of the immunogenic fragment of SEQ ID NO: 177. Preferably the only US28-derived sequence in said peptide or polypeptide is, or are, the sequence(s) of SEQ ID NO: 6 and/or 7 or the sequence of the immunogenic fragment of SEQ ID NO: 6 and/or 7, and optionally also the sequence of SEQ ID NO: 177 or the sequence of the immunogenic fragment of SEQ ID NO: 177.

A peptide or polypeptide comprising, consisting essentially of, or consisting of, an immunogenic fragment of the reference sequence SEQ ID NO: 177, in accordance with the thirteenth or fourteenth aspect of the present invention comprises less than the full sequence of the reference sequence, and preferably comprises at least 3, 4, 5, 6, 7, 8, 9, 10 or 11 consecutive amino acids of the reference sequence of SEQ ID NO: 177.

An immunogenic fragment of the reference sequence SEQ ID NO: 6 and/or 7, in accordance with the fourteenth aspect of the present invention, respectively, comprising less than the full sequence of the reference sequence, and preferably comprising at least 3, 4, 5, 6, 7, 8, 9, 10, 11, 12, 13, 14, 15, or 16 consecutive amino acids of the reference sequence.

In one embodiment, an immunogenic fragment of the peptide or polypeptide of the fourteenth aspect of the present invention, comprises, consists essentially of, or consists of, a sequence that is common to, and present within, both of SEQ ID NO: 6 and SEQ ID NO: 7.

Also provided by the thirteenth and fourteenth aspects of the present invention are peptides or polypeptides comprising, consisting, or consisting essentially of:
(i) an immunogenic fragment or variant of reference sequence TDVLNQSKPVTL (SEQ ID NO: 177) that comprises the sequence of an epitope within SEQ ID NO: 177 that is bound by any of antibodies 4H3C3, 7B1F3 and/or 2F5B11, as described herein (by which is included also an scFv comprising a V_{H} polypeptide sequence having the V_{H} sequence of 4H3C3, 7B1F3 and/or 2F5B11, as defined by SEQ ID Nos: 187, 211 and 233, respectively, and a V_{L} polypeptide sequence having the V_{L} sequence of 4H3C3, 7B1F3 and/or 2F5B11 as defined by SEQ ID Nos: 189, 213 and 235, respectively); preferably said peptides or polypeptides are bound specifically by 4H3C3, 7B1F3 and/or 2F5B11.
(ii) an immunogenic fragment or variant of a reference sequence selected from TKKNNQCMTDYDYLEVS (SEQ ID NO: 6) and/or TKKDNQCMTDYDYLEVS (SEQ ID NO: 7), respectively, wherein the immunogenic fragment or variant comprises the sequence of the epitope within ECD3 of US28 that is bound by any of antibodies 1D3, 1C10, 1A10, 1G9 and/or 1E8, as described herein (by which is included also an scFv comprising a V_{H} polypeptide sequence having the V_{H} sequence of 1D3, 1C10, 1A10, 1G9 and/or 1E8, as defined by SEQ ID Nos: 12, 104, 122, 68 and 88, respectively, and a V_{L} polypeptide sequence having the V_{L} sequence of 1D3, 1C10, 1A10, 1G9 and/or 1E8 as defined by SEQ ID Nos: 18, 108, 126, 72 and 92, respectively). Preferably said peptides or polypeptides are bound specifically by 1D3, 1C10, 1A10, 1G9 and/or IES.

A fifteenth aspect of the present invention provides a combination of at least two (for example, three) distinct peptides and/or polypeptides, comprising a first peptide or polypeptide and a second peptide or polypeptide (and optionally at least a third peptide or polypeptide), wherein:
(a) the first peptide or polypeptide comprises a comprises, consists essentially of, or consists of, the sequence TDVLNQSKPVTL (SEQ ID NO: 177), or an immunogenic fragment thereof, such as an immunogenic fragment as defined by the thirteenth aspect of the present invention; and
(b) the second peptide or polypeptide comprises a comprises, consists essentially of, or consists of, the sequence TKKNNQCMTDYDYLEVS (SEQ ID NO: 6), or an immunogenic fragment thereof, such as an immunogenic fragment as defined by the fourteenth aspect of the present invention, with the proviso that said immunogenic fragment comprises at least the 4N amino acid of SEQ ID NO: 6; or
(c) the second peptide or polypeptide comprises a comprises, consists essentially of, or consists of, the sequence TKKDNQCMTDYDYLEVS (SEQ ID NO: 7), or an immunogenic fragment thereof, such as an immunogenic fragment as defined by the fourteenth aspect of the present invention, with the proviso that said immunogenic fragment comprises at least the 4D amino acid of SEQ ID NO: 7; and optionally wherein the combination comprises the third polypeptide and, if the second peptide or polypeptide is as defined by option (b) then the third peptide or polypeptide corresponds to option (c), or wherein if the second peptide or polypeptide is as defined by option (c) then the third peptide or polypeptide corresponds to option (b).

A sixteenth aspect of the present invention provides a fusion protein comprising, consisting essentially of, or consisting of, a first amino acid sequence fused, either directly or via one or more linker amino acid sequences, to a second amino acid sequence, wherein the first amino acid sequence is the sequence of a peptide or polypeptide as defined by the thirteenth or fourteenth aspect of the present invention; and the second amino acid sequence is a fusion partner.

Optionally, the fusion partner is a carrier protein, such as a carrier protein that is selected to provide a fusion protein that is suitable for immunisation and generation of antibodies against the first amino acid sequence. For example, the carrier protein may be selected from the group consisting of keyhole limpet hemocyanin (KLH), HSA (human serum albumin), BSA (bovine serum albumin), OVA (ovalbumin), tetanus toxoid (TT), diphtheria toxoid (DT), a genetically modified cross-reacting material (CRM) of diphtheria toxin, meningococcal outer membrane protein complex (OMPC) and H. influenzae protein D (HiD).

A seventeenth aspect of the present invention provides a combination of at least two (for example, three) distinct fusion proteins, comprising a first fusion protein, and a second fusion protein (and optionally at least a third distinct fusion protein), wherein:
(a) the first fusion protein according to the sixteenth aspect of the present invention comprises, as the first amino acid sequence of the first fusion protein, a sequence that comprises a comprises, consists essentially of, or consists of, the sequence TDVLNQSKPVTL (SEQ ID NO: 177), or an immunogenic fragment or variant thereof; and
(b) the second fusion protein according to the sixteenth aspect of the present invention comprises, as the first amino acid sequence of the first fusion protein, a sequence that comprises a comprises, consists essentially of, or consists of, the sequence TKKNNQCMTDYDYLEVS (SEQ ID NO: 6), or an immunogenic fragment or variant thereof, with the proviso that said immunogenic fragment or variant includes the 4N amino acid of SEQ ID NO: 6; or
(c) the second fusion protein according to the sixteenth aspect of the present invention comprises, as the first amino acid sequence of the second fusion protein, a sequence that comprises, consists essentially of, or consists of, the sequence TKKDNQCMTDYDYLEVS (SEQ ID NO: 7), or an immunogenic fragment or variant thereof, with the proviso that said immunogenic fragment or variant includes the 4D amino acid of SEQ ID NO: 7; and
optionally wherein the combination comprises the third distinct fusion protein and, if the second fusion protein is as defined by option (b) then the third fusion protein corresponds to option (c), or wherein if the second fusion protein is as defined by option (c) then the third fusion protein corresponds to option (b).

An eighteenth aspect of the present invention provides a conjugate, comprising a moiety conjugated to a peptide or polypeptide as defined by either or both of the thirteenth and fourteenth aspects of the present invention, or to a fusion protein as defined by the sixteenth aspect of the present invention.

The moiety of said conjugate may be conjugated directly to the peptide or polypeptide as defined by either or both of the thirteenth and fourteenth aspects of the present invention, or to the fusion protein as defined by the sixteenth aspect of the present invention. Alternatively, the moiety of said conjugate may be conjugated indirectly, such as via a linker, to the peptide or polypeptide as defined by either or both of the thirteenth and fourteenth aspects of the present invention, or to the fusion protein as defined by the sixteenth aspect of the present invention.

Optionally, the moiety may be a carrier, for example a carrier protein, such as a carrier selected from KLH (keyhole limpet hemocyanin), HSA (human serum albumin), BSA (bovine serum albumin), OVA (ovalbumin), tetanus toxoid (TT), diphtheria toxoid (DT), a genetically modified cross-reacting material (CRM) of diphtheria toxin, meningococcal outer membrane protein complex (OMPC) and H. influenzae protein D (HiD).

A nineteenth aspect of the present invention provides a combination of at least two (for example, three) distinct conjugates, wherein the combination comprises:
(a) a first conjugate according to the eighteenth aspect of the present invention, wherein the first conjugate comprises, consists essentially of, or consists of, a moiety conjugated to a peptide or polypeptide, wherein the peptide or polypeptide comprises, consists essentially of, or consists of, the sequence TDVLNQSKPVTL (SEQ ID NO: 177), or an immunogenic fragment or variant thereof; and
(b) a second conjugate according to the eighteenth aspect of the present invention, wherein the second conjugate comprises, consists essentially of, or consists of, a moiety conjugated to a peptide or polypeptide, wherein the peptide or polypeptide comprises, consists essentially of, or consists of, the sequence TKKNNQCMTDYDYLEVS (SEQ ID NO: 6), or an immunogenic fragment or variant thereof, with the proviso that said immunogenic fragment or variant includes the 4N amino acid of SEQ ID NO: 6; or
(c) a second conjugate according to the eighteenth aspect of the present invention, wherein the second conjugate comprises, consists essentially of, or consists of, a moiety conjugated to a peptide or polypeptide, wherein the peptide or polypeptide comprises, consists essentially of, or consists of, the sequence TKKDNQCMTDYDYLEVS (SEQ ID NO: 7), or an immunogenic fragment or variant thereof, with the proviso that said immunogenic fragment or variant includes the 4D amino acid of SEQ ID NO: 7; and
optionally wherein the combination comprises a third distinct conjugate and, if the second conjugate is as defined by option (b) then the third conjugate corresponds to option (c), or wherein if the second conjugate is as defined by option (c) then the third conjugate corresponds to option (b).

A twentieth aspect of the present invention provides a method of producing a conjugate according to the nineteenth aspect of the present invention, the method comprising the steps of:
(a) providing a peptide or polypeptide as defined by either or both of the thirteenth and/or fourteenth aspects of the present invention, a combination thereof as defined by the fifteenth aspect of the present invention, or a fusion protein as defined by the sixteenth aspect of the present invention; and
(b) conjugating a moiety thereto.

A twenty-first aspect of the present invention provides a method of producing a combination of at least two (for example, three) distinct conjugates as defined by the nineteenth aspect of the present invention.

In one embodiment, the method comprising the steps of: (a) providing or producing the first conjugate, as defined by the eighteenth aspect of the present invention, by a method according to the twentieth aspect of the present invention; (b) providing or producing the second conjugate, as defined by the eighteenth aspect of the present invention, by a method according to the twentieth aspect of the present invention (wherein the first and second conjugates are distinct); (c) optionally providing or producing the third conjugate, as defined by the eighteenth aspect of the present invention, by a method according to the twentieth aspect of the present invention (wherein the first, second and third conjugates are distinct); and (d) combining the first and second, and optionally third, conjugates, thereby to form a combination according to the nineteenth aspect of the present invention.

In another embodiment, the method comprising the steps of: (a) providing a combination of at least two (for example, three) distinct peptides and/or polypeptides according to fifteenth aspect of the present invention, or a combination of at least two (for example, three) distinct fusion proteins according to the seventeenth aspect of the present invention; and (b) conjugating a moiety to the combination of at least two (for example, three) distinct peptides and/or polypeptides according to fifteenth aspect of the present invention, or to the combination of at least two (for example, three) distinct fusion proteins according to the seventeenth aspect of the present invention, thereby to form a combination according to the nineteenth aspect of the present invention.

The methods of the twentieth or twenty-first aspect of the present invention optionally comprise the step of isolating the thus-produced conjugate or combination of conjugates.

A twenty-second aspect of the present invention provides an isolated conjugate, or combination of conjugates, that is obtained, or obtainable, by the method of any of twentieth or twenty-first aspect of the present invention, optionally, wherein the isolated conjugate is further formulated for administration to a subject.

A twenty-third aspect of the present invention provides nucleic acid molecule, or combination of multiple distinct nucleic acid molecules, wherein the nucleic acid molecule comprises, or the combination of multiple distinct nucleic acid molecules collectively comprises, one or more nucleic acid sequences that, individually or in combination, encode one or more peptides and/or polypeptides according to either or both of the thirteenth and/or fourteenth aspects of the present invention, a combination of at least two (for example, three) distinct peptides and/or polypeptides according to the fifteenth aspect of the present invention, a fusion protein according to the sixteenth aspect of the present invention, and/or a combination of at least two (for example, three) distinct fusion proteins according to the seventeenth aspect of the present invention.

The, or each, nucleic acid molecule according to the twenty-third aspect of the present invention may, for example, be each independently selected from a DNA or RNA molecule. The, or each, nucleic acid molecule according to the twenty-third aspect of the present invention may, for example, be each independently selected from a single-stranded or a double-stranded nucleic acid molecule.

A twenty-fourth aspect of the present invention provides a vector comprising a nucleic acid molecule, or combination of multiple distinct nucleic acid molecules, according to the twenty-third aspect of the present invention. Any vector may be used, although without limitation, said vector may optionally be selected from the group consisting of a retroviral vector, a plasmid, a lentivirus vector, and an adenoviral vector.

A twenty-fifth aspect of the present invention provides a cell comprising the nucleic acid molecule, or combination of multiple distinct nucleic acid molecules, according to the twenty-third aspect of the present invention, or the vector according to the twenty-fourth aspect of the present invention. Optionally, the cell expresses one or more peptide or polypeptide selected from either or both of the thirteenth and/or fourteenth aspects of the present invention, a combination of at least two (for example, three) distinct peptides and/or polypeptides according to the fifteenth aspect of the present invention, a fusion protein according to the sixteenth aspect of the present invention, and/or a combination of at least two (for example, three) distinct fusion proteins according to the seventeenth aspect of the present invention.

A twenty-sixth aspect of the present invention provides a cell that is exposed to, and/or comprising, one or more peptide or polypeptide selected from either or both of the thirteenth and/or fourteenth aspects of the present invention, a combination of at least two (for example, three) distinct peptides and/or polypeptides according to the fifteenth aspect of the present invention, a fusion protein according to the sixteenth aspect of the present invention, and/or a combination of at least two (for example, three) distinct fusion proteins according to the seventeenth aspect of the present invention, a conjugate according to eighteenth aspect of the present invention, a combination of at least two (for example, three) distinct conjugates according the nineteenth aspect of the present invention, a nucleic acid molecule, or combination of multiple distinct nucleic acid molecules, according to the twenty-third aspect of the present invention, and/or a vector according to the twenty-fourth aspect of the present invention.

A twenty-seventh aspect of the present invention provides a method of isolating and/or enriching cells comprising a T cell receptor (TCR) with specificity to a first epitope within a polypeptide consisting of the amino acid sequence of TDVLNQSKPVTL (SEQ ID NO: 177) in ECD1 (and optionally a second epitope in ECD3) of US28 (e.g. naturally occurring T cells, or recombinant cells expressing a CAR according to the first aspect of the present invention, for example CAR T-cells, CAR NK-cells and/or CAR-macrophages), wherein the method comprises the step of using one or more agents to isolate and/or enrich cells with binding specificity to the sequence of SEQ ID Nos: 177 or an immunogenic fragment thereof, and optionally also with binding specificity to one or both of the sequences of SEQ ID Nos: 6 and/or 7, or immunogenic fragments of either

wherein the one or more agents is or are selected from the group consisting of a peptide or polypeptide according to either or both of the thirteenth and/or fourteenth aspects of the present invention, a combination of at least two (for example, three) distinct peptides and/or polypeptides according to the fifteenth aspect of the present invention, a fusion protein according to the sixteenth aspect of the present invention, and/or a combination of at least two (for example, three) distinct fusion proteins according to the seventeenth aspect of the present invention, a conjugate according to eighteenth aspect of the present invention, and/or a combination of at least two (for example, three) distinct conjugates according the nineteenth aspect of the present invention.

In one embodiment of the method of the twenty-seventh aspect of the present invention, the sequences can be formulated as an MHC tetramer, for example a Class I MHC tetramer for antigen-specific CD8⁺ T cells detection, a Class II MHC tetramer for antigen-specific CD4⁺ T cells detection, or a fluorophore-labelled tetramer for flow cytometry or fluorescence microscopy. Optionally, the T-cell may be selected from the group consisting of CD8⁺ T cells, CD4+ T cells, effector T cells, helper T cells, memory T cells, cytotoxic T lymphocytes (CTLs), EBV-specific T cell receptor (TCR) or γδ-T cell subtypes.

A twenty-eighth aspect of the present invention provides an MHC tetramer comprising a peptide or polypeptide according a peptide or polypeptide according to either or both of the thirteenth and/or fourteenth aspects of the present invention, a combination of at least two (for example, three) distinct peptides and/or polypeptides according to the fifteenth aspect of the present invention, optionally wherein the or each peptide comprises or corresponds to SEQ ID NO: 177, SEQ ID NO: 6 and/or SEQ ID NO: 7, or an immunogenic fragment of either or both, for example wherein the MHC tetramer is a Class I MHC tetramer for antigen-specific CD8+ T cells detection, a Class II MHC tetramer for antigen-specific CD4+ T cells detection, a fluorophore-labelled tetramer for flow cytometry or fluorescence microscopy. The MHC tetramer may further be used in isolating and/or enriching cells comprising a T cell receptor (TCR) with specificity to a first epitope within a polypeptide consisting of the amino acid sequence of TDVLNQSKPVTL (SEQ ID NO: 177) in ECD1 and/or ECD3 of US28, for example, a T-cell selected from the group consisting of CD8⁺ T cells, CD4⁺ T cells, effector T cells, helper T cells, memory T cells, cytotoxic T lymphocytes (CTLs), EBV-specific T cell receptor (TCR) or γδ-T cell subtypes and/or a recombinant cell expressing a CAR according to the first aspect of the present invention, for example a CAR T-cell, CAR NK-cell and/or CAR-macrophage.

A twenty-ninth aspect of the present invention provides a vaccine composition suitable for use in vaccinating against, reducing the risk of, preventing, or combating a disease or condition associated with human cytomegalovirus (HCMV). The vaccine may be an active or passive vaccine. An active vaccine according to the twenty-ninth aspect of the present invention may trigger an immune response directed to a first epitope present within a polypeptide consisting of the amino acid sequence of TDVLNQSKPVTL (SEQ ID NO: 177) within ECD1 of a US28 protein of HCMV. Optionally, the active vaccine also triggers and/or provides an immune response directed to a second epitope present within ECD3) of a US28 protein of HCMV. A passive vaccine according to the twenty-ninth aspect of the present invention may be a composition that provides an immune response directed to a first epitope present within a polypeptide consisting of the amino acid sequence of TDVLNQSKPVTL (SEQ ID NO: 177) within ECD1 of a US28 protein of HCMV. Optionally, the passive vaccine also provides an immune response directed to a second epitope present within ECD3) of a US28 protein of HCMV. As discussed above in the context of other aspects of the present invention, the ECD1, SEQ ID NO: 177 and ECD3 of the US28 protein comprises, consists essentially of, or consist of, an amino acid sequence presented in the US28 protein encoded by a strain of HCMV at positions corresponding to positions 1 to 37, 26 to 37, and 167 to 183, respectively, of the US28 protein encoded by the DB strain of human cytomegalovirus (HCMV) as set forth in SEQ ID NO: 5.

In some embodiments, the vaccine composition of the twenty-ninth aspect of the present invention triggers and/or provides an immune response:
(a) to one or more epitopes present entirely within a polypeptide consisting of the amino acid sequence of TDVLNQSKPVTL (SEQ ID NO: 177) within ECD1 of the US28 protein of HCMV, and/or to one or more epitopes present entirely within ECD3 of the US28 protein of HCMV;
(b) to one or more linear epitopes within a polypeptide consisting of the amino acid sequence of TDVLNQSKPVTL (SEQ ID NO: 177) within ECD1 of the US28 protein of HCMV, and/or to one or more linear epitopes present within ECD3) of the US28 protein of HCMV;
(c) to one or more epitopes within ECD3 of a US28 protein of HCMV that is an epitope, or are epitopes, present in identical form in both the 4D-variant strains and 4N-variant strains of HCMV, wherein the 4D-variant strain of HCMV encodes a US28 protein comprising an ECD3 having the sequence of TKKDNQCMTDYDYLEVS (SEQ ID NO: 7) and wherein the 4N-variant strain of HCMV encodes a US28 protein comprising an ECD3 having the sequence of TKKNNQCMTDYDYLEVS (SEQ ID NO: 6));
(d) wherein the immune response that is triggered or provided by the vaccine is HCMV strain agnostic to multiple HCMV strains that show inter-strain sequence variation in one or more positions in the region of positions 1 to 25 of ECD1 (such as any one or more of positions 8, 15, 18, 19, 24, 25, including but not limited to the sequence variations listed in Table 3 of the present application), for example wherein the multiple strains are two, three, four, five, six, seven, eight, nine or ten different strains selected from the group consisting of DE, Toledo, Towne, VR1814, TB40/E, Merlin, AD169, VHL/E, Davis and BL; and/or
(e) wherein the immune response that is triggered or provided by the vaccine is HCMV strain agnostic to the 4D-variant strains and 4N-variant strains of HCMV), and triggers and/or provides an immune response that is directed to one or more of the 4D-variant HCMV strains selected from Towne, VR1814, TB40/E, Merlin, JP, Ad169, VHL/E, BL, AF1 and DAVIS and is also directed to one or more of the 4N-variant HCMV strains selected from Toledo, TR and DB.

Said vaccine composition may be a passive vaccine, and/or optionally comprise: (a) one or more binding molecules according to the first aspect of the present invention, (b) one or more functional fragments of said one or more binding molecules as defined by the first aspect of the present invention, (c) one or more isolated binding molecules according to the seventh aspect of the present invention, (d) one or more nucleic acid molecules, or combination of multiple distinct nucleic acid molecules, according to the second aspect of the present invention, (e) one or more vectors according to the third aspect of the present invention, (f) one or more cells according to the fourth aspect of the present invention, (g) one or more conjugates according to the eighth aspect of the present invention, and/or (h) one or more isolated conjugates according to the tenth aspect of the present invention.

Alternatively, said vaccine composition may be an active vaccine, and/or optionally comprise:
(a) one or more peptides or polypeptides according to either or both of the thirteenth and/or fourteenth aspects of the present invention, a combination of at least two (for example, three) distinct peptides and/or polypeptides according to the fifteenth aspect of the present invention, a fusion protein according to sixteenth aspect of the present invention, a combination of at least two (for example, three) distinct fusion proteins according to the seventeenth aspect of the present invention, a conjugate according to the eighteenth aspect of the present invention, and/or a combination of at least two (for example, three) distinct conjugates according to the nineteenth aspect of the present invention;
(b) one or more nucleic acid molecules, or combination of multiple distinct nucleic acid molecules, according to the twenty-third aspect of the present invention, and/or the vector according to the twenty-fourth aspect of the present invention; and/or
(c) a cell, such as an antigen-presenting cell (e.g. a dendritic cell), or a homogeneous or heterogeneous population of said cells, wherein the or each of said cells is loaded with one or more of the following: a peptide or polypeptide according to either or both of the thirteenth and/or fourteenth aspects of the present invention, a combination of at least two (for example, three) distinct peptides and/or polypeptides according to the fifteenth aspect of the present invention, a fusion protein according to sixteenth aspect of the present invention, a combination of at least two (for example, three) distinct fusion proteins according to the seventeenth aspect of the present invention, a conjugate according to the eighteenth aspect of the present invention, a combination of at least two (for example, three) distinct conjugates according to the nineteenth aspect of the present invention, one or more nucleic acid molecules, or combination of multiple distinct nucleic acid molecules, according to the twenty-third aspect of the present invention, and/or the vector according to the twenty-fourth aspect of the present invention.

A thirtieth aspect of the present invention provides a method of vaccinating against, reducing the risk of, preventing, and/or combating a disease or condition associated with HCMV, the method comprising administering to a subject a vaccine according to the twenty-ninth aspect of the present invention.

The thirtieth aspect of the present invention provides a vaccine according to the twenty-ninth aspect of the present invention for use in vaccinating against, reducing the risk of, preventing, and/or combating a disease or condition associated with HCMV in a subject.

The thirtieth aspect of the present invention provides for the use of a vaccine according to the twenty-ninth aspect of the present invention in the manufacture of a medicament for vaccinating against, reducing the risk of, preventing, and/or combating a disease or condition associated with HCMV.

Said disease or condition associated with HCMV may, for example, be a disease or condition associated with HCMV as disclosed above in the context of the eleventh aspect of the present invention. Optionally, the disease or condition is a latent HCMV infection, or is a disease or condition associated with a latent HCMV infection.

In some embodiments, the method of vaccinating against, reducing the risk of, preventing, and/or combating a disease or condition associated with HCMV may comprise administering the vaccine to the subject only once.

In other embodiments, the method of vaccinating against, reducing the risk of, preventing, and/or combating a disease or condition associated with HCMV may comprise administering the vaccine to the subject vaccine twice or multiple times. For example, in the embodiment in which the vaccine is an active vaccine, it may be appropriate to separately administer a primary dose, and a subsequent booster dose, to the subject.

A thirty-first aspect of the present invention provides a method of assessing one or more biological conditions and/or biological characteristics of a subject and/or of ex vivo biological material, wherein the method comprises: (a) contacting the subject and/or the ex vivo biological material with a binding molecule, for example as defined by the first aspect of the present invention, or a conjugate, for example as defined by the eighth or tenth aspect of the present invention; and (b) making an assessment of the subject and/or the ex vivo biological material based on a direct and/or indirect measurement of the binding of the binding molecule or conjugate to the subject and/or the ex vivo biological material.

In some embodiments, the method of assessing (which can include diagnosing) one or more biological conditions and/or biological characteristics of a subject and/or of ex vivo biological material is by In Situ Hybridisation (ISH) for the specific detection of one or more nucleic acid sequences encoded by HCMV, most preferably wherein said ISH does not detect nucleic acid sequences encoded by a healthy (i.e. not infected by HCMV) subject and/or a healthy ex vivo biological material. By way of non-limiting example, the ISH may use nucleic acid sequences as discussed in the Examples of the present application. In some embodiments, the biological condition is cancer and/or the biological characteristics are related to cancer. In some embodiments, the cancer is selected from one or more of the cancers specified herein, optionally wherein the cancer is not glioblastoma. In some embodiments, the cancer is selected from the group consisting of: breast cancer (for example HER2+ breast cancer, or triple negative breast cancer), astrocytoma, glioblastoma, adrenal cortical cancer, kidney cancer, cardiac sarcoma, liver cancer, and vascular smooth muscle cancers; optionally wherein the cancer is not glioblastoma.

In some embodiments, the method of assessing (which can include diagnosing) one or more biological conditions and/or biological characteristics of a subject and/or of ex vivo biological material is by immunohistochemistry (IHC) for the specific detection of one or more protein sequences encoded by HCMV, most preferably wherein said IHC does not detect protein sequences encoded by a healthy (i.e. not infected by HCMV) subject and/or a healthy ex vivo biological material. In some embodiments, the IHC uses a binding molecule that allows for the specific detection of one or more protein sequences only expressed, or only surface expressed, by a latent HCMV infection. In some embodiments, the IHC uses a binding molecule that allows for the specific detection of one or more protein sequences only expressed, or only surface expressed, by a lytic HCMV infection. In some embodiments, the IHC uses a binding molecule that allows for the specific detection of one or more protein sequences expressed, for example surface expressed, by a both lytic and latent HCMV infections. In some embodiments, the IHC may be performed (e.g. using biological tissue in which the cells have not been permeabilised) to allow only for the detection of cell surface-expressed proteins. In other embodiments, the IHC may be performed (e.g. using biological tissue in which the cells have been permeabilised) to allow for the detection of intracellular proteins. By way of non-limiting examples, the IHC may use an antibody-based molecule for the specific detection of one or more protein sequences encoded by HCMV, as discussed in the Examples of the present application and/or using one or more of the binding molecules of the present invention. In some embodiments, the biological condition is cancer and/or the biological characteristics are related to cancer. In some embodiments, the cancer is selected from one or more of the cancers specified herein, optionally wherein the cancer is not glioblastoma. In some embodiments, the cancer is selected from the group consisting of: breast cancer (for example HER2+ breast cancer, or triple negative breast cancer), astrocytoma, glioblastoma, adrenal cortical cancer, kidney cancer, cardiac sarcoma, liver cancer, and vascular smooth muscle cancers; optionally wherein the cancer is not glioblastoma.

A subject and/or living ex vivo biological material in which HCMV infection has been positively identified by the thirty-second aspect of the present invention can be an exemplary subject and/or material that can be treated in accordance with the other aspects of the present invention as described herein.

A thirty-second aspect of the present invention provides a method of combating a HCMV infection (such as a latent HCMV infection and/or a lytic HCMV infection and/or a multi-strain HCMV infection) in living ex vivo biological material, the method comprising contacting the living ex vivo biological material with any one or more agents selected from the group consisting of:
i. a binding molecule according to the first aspect of the present invention,
ii. a functional fragment of said binding molecule as defined by the first aspect of the present invention,
iii. an isolated binding molecule according to the seventh aspect of the present invention,
iv. a nucleic acid molecule, or combination of multiple distinct nucleic acid molecules, according to the second aspect of the present invention,
v. a vector according to the third aspect of the present invention,
vi. a cell according to the fifth aspect of the present invention,
vii. a conjugate according to the eighth aspect of the present invention, and
viii. an isolated conjugate according to the tenth aspect of the present invention.

The thirty-second aspect of the present invention also provides living ex vivo biological material that is obtained, or obtainable, by the method of this aspect.

In one embodiment of the method of the thirty-second aspect of the present invention, and/or the living ex vivo biological material obtained, or obtainable, thereby, the ex vivo living biological material comprises, consists essentially of, or consists of, living ex vivo biological material selected from the group that includes: one or more types of ex vivo cells; one or more types of ex vivo cell cultures; one or more types of ex vivo tissues; one or more types of ex vivo tissue cultures; one or more types of ex vivo organoids; one or more types of ex vivo organoid cultures; one or more types of ex vivo organs; and/or one or more types of ex vivo organ cultures.

A thirty-third aspect of the present invention provides a method of treating a subject in need thereof, comprising administering ex vivo living biological material as defined by the thirty-second aspect of the present invention, to the subject.

For example, the method may be a method of transplantation of the ex vivo living biological material, such as an organ or tissue transplant. Said method can be used to prevent, or reduce the risk of, the transmission of and HCMV infection, or a disease or condition associated with an HCMV infection in the recipient of the transplant.

A thirty-fourth aspect of the present invention provides a method of screening for a binding molecule having binding specificity and/or binding affinity to a first epitope within a polypeptide consisting of the amino acid sequence of TDVLNQSKPVTL (SEQ ID NO: 177) within ECD1 of a US28 protein of human cytomegalovirus (HCMV), the method comprising:
(a) providing one or more peptides or polypeptides comprising, consisting essentially of, or consisting of, the sequence TDVLNQSKPVTL. (SEQ ID NO: 177), or an immunogenic fragment thereof,
preferably wherein the only US28-derived sequence present within said one or more peptides or polypeptides is the sequence of SEQ ID NO: 177 or an immunogenic fragment thereof, such as a fragment that is bound by antibody 4H3C3, 7B1F3 and/or 2F5B11;
(b) providing one or more candidate binding molecules;
(c) determining the binding specificity and/or binding affinity of one or more candidate binding molecules to the one or more peptides, thereby to select one or more binding molecules having binding specificity to the first epitope within a polypeptide consisting of the amino acid sequence of SEQ ID NO: 177; and
optionally, wherein the method further involves screening said binding molecules for binding specificity to an epitope within extracellular domain 3 (ECD3) of a US28 protein of human cytomegalovirus (HCMV), by additional method steps (which may be performed either before, or after steps (a)-(c)), said additional method steps comprising:
(i) providing one or more peptides or polypeptides corresponding an amino acid sequence present in ECD3 of the US28 protein,
preferably wherein the only US28-derived sequence present within said one or more peptides is the sequence of SEQ ID NO: 6 and/or 7, or an immunogenic fragment of either, such as a fragment that is bound by antibody 1D3, 1C10, 1A10, 1G9, and/or 1E8;
(ii) providing one or more candidate binding molecules, optionally wherein steps (a)-(c) are performed before steps (i)-(iii) and wherein said candidate binding molecules for step (ii) are a product of step (c);
(iii) determining the binding specificity and/or binding affinity of one or more candidate binding molecules to the one or more peptides or polypeptides corresponding an amino acid sequence present in ECD3 of the US28 protein, optionally wherein steps (a)-(c) are performed after steps (i)-(iii) and wherein said candidate binding molecules for step (b) are a product of step (iii) of this claim.

A thirty-fifth aspect of the present invention provides a method of producing a composition that comprises multiple copies of a binding molecule, said method comprising causing the reproduction of a selected candidate binding molecule that has been selected in accordance with the method of the thirty-fourth aspect of the present invention.

A thirty-sixth aspect of the present invention provides a method of assessing a selected candidate binding molecule that has been selected in accordance with the method of the thirty-fourth aspect of the present invention and/or produced in accordance with the method of the thirty-fifth aspect of the present invention, said method comprising identifying the structure(s) within the selected candidate binding molecule that provides its binding characteristics (in particular, the binding specificity and/or binding affinity to a first epitope within a polypeptide consisting of the amino acid sequence of TDVLNQSKPVTL (SEQ ID NO: 177) within ECD1, and optionally the binding specificity and/or binding affinity to a second epitope within ECD3 of a US28 protein of human cytomegalovirus (HCMV)), for example, by identifying the, or each, CDR sequence in a selected candidate binding molecule that is an antibody or CAR.

A thirty-seventh aspect of the present invention provides a method of producing a composition that comprises multiple copies of a binding molecule having binding specificity and/or binding affinity to a first epitope within a polypeptide consisting of the amino acid sequence of TDVLNQSKPVTL (SEQ ID NO: 177) within ECD1 (and optionally to a second epitope within ECD3) of a US28 protein of human cytomegalovirus (HCMV), wherein said binding molecule comprises the, or each, of the structure(s) (e.g. CDR sequences) that have been identified within a selected candidate binding molecule as providing its binding characteristics, in accordance with the method of the thirty-sixth aspect of the present invention, said method comprising causing the reproduction of the binding molecule.

The thirty-seventh aspect of the present invention further provides a composition of binding molecule obtained by the same aspect.

### DESCRIPTION OF THE FIGURES

FIG. 1. Western blot analysis was used to verify the US28 protein expression in the transformed CHO-US28-A1 cells by using an anti-HIS Ab. From the left, on the first line: the ladder, on the second line: protein extract from the CHO control cells, on the third line: protein extract from the CHO-US28-A1 cells and on the fourth line: the 6HIS positive control. The C-terminal part of the US28-A1 construct, which was transfected into the CHO-US28-A1 cells, contains a 6HIS tag. Staining with the anti-HIS antibody, that binds on the 6HIS tag marked out a specific band at ˜41 kDa when compared with the ladder, which is equivalent with the earlier reported size of the HCMV US28 protein. This band, which is circled in the picture, was not present in the control CHO-cells, therefore verifying the expression of US28 in the transfected CHO-US28-A1 cells but not in the control CHO cells. The 6HIS positive control is positive indicating that the 6HIS Ab binds to its target as expected.

FIG. 2. Flowcytometry analysis showing surface binding of the US28-13-5G6-1D3 antibody clone on CHO-US28-A1 cells. From the left, blank control, 2nd antibody control and clone US28-13-5G6-1D3 Ab binding on the surface of the CHO-US28-A1 cells. The US28-13-5G6-1D3 Ab bound to 53.4% of the CHO-US28-A1 cells, whereas the blank and secondary antibody controls showed low binding: 0.11% and 1.63% respectively.

FIG. 3. The positive surface binding of the clone US28-13-5G6-1D3 on CHO-US28-A1 cells was further validated by gradient dilution in FACS analysis. From the left, the clone US28-13-5G6-1D3 Ab surface binding to the CHO-US28-A1 cells in dilutions 1:20, 1:50, 1:200 (w/v).

FIG. 4. The positive surface binding of the clone US28-13-5G6-1D3 on CHO-US28-A1 cells was compared with the US28 negative CHO control cells. The clone US28-13-5G6-1D3 Ab bound 15-fold more to the surface of the US28 positive CHO-US28-A1 cells than to the US28 negative CHO cells in 1:50 (w/v).

FIG. 5. The original ELISA analysis showed equal qualitative binding of the US28-13-5G6-1D3 antibody clones on both Bio-Peptide-1 (US28 ECD3 genotype 4N) and Bio-Peptide-2 (US28 ECD3 genotype 4D). Since no other mutations in the ECD3 has been observed, these results indicate that the binding of the US28-13-5G6-1D3 Ab to its target is HCMV strain agnostic.

FIG. 6. Qualitative ELISA analysis showing binding of the recombinant antibody product US28-13-5G6-1D3 rAb to Bio-Peptides 1 and 2. These results show strong and equal binding of the generated antibody to both genetic variants of US28 ECD3 confirming that the recombinant antibody binds well to its target and maintains the HCMV strain agnostic binding properties.

FIG. 7. Surface binding of 13-5G6-1D3 rAb on CHO-US28-A1 cells, and on respective control CHO cells were validated by using FACS analysis. The bars showing results for CHO cells are colored with dark grey whereas the bars showing results for CHO-US28-A1 cells are colored with pale grey color. From the left, the first bar shows measurement of the blank control with no antibody for the CHO-US28-A1 and CHO cells, respectively; the second bar from the left shows surface binding of the anti-mouse IgG-Alexa 488 secondary Ab alone on both cell types; the third bar from the left shows surface binding of 13-5G6-1D3 rAb in 1:10 dilution (w/v) on both cell types; the fourth bar from the left shows 13-5G6-1D3 rAb surface binding in 1:50 dilution (w/v) on CHO-US28-A1 cells only; and the fifth bar from the left shows the surface binding of 13-5G6-1D3 rAb in 1:100 dilution (w/v) on CHO-US28-A1 cells only. The y-axis shows the binding to the percent of cells. Based on these results, the optimal staining dilution for 13-5G6-1D3 rAb is 1:50 w/v (the third bar from the left). The surface binding of the 13-5G6-1D3 rAb to CHO cells was only measured in 1:10 dilution (w/v) and was 0.77% after removing the binding to blank and anti-mouse IgG-Alexa 488 secondary Ab controls. These results are consistent with the previous results obtained by using the antibody from 13-5G6-1D3 clone and shows highly specific surface binding of the 13-5G6-1D3 rAb on CHO-US28-A1 cells, which is >21-fold more than to the normal CHO cells.

FIG. 8. Binding of the US28-13-5G6-1D3 rAb on the surface of the HCMV Ad169 infected MRC-5 cell population but not on the Mock cells were shown by FACS analysis. A. The upper row shows the surface binding of the US28-13-5G6-1D3 rAb on Mock versus HCMV Ad169 infected MRC-5 cells showing highly specific binding on the HCMV infected cells. B. To verify that the 13-5G6-1D3 rAb was binding to the surface of the HCMV infected cell population, another set of HCMV Ad169 infected and Mock cells were permeabilized and stained with the commercial antibody MAB810X against the HCMV Major Immediate Early (IE) antigen, which is an intracellular protein known to be expressed early during the lytic HCMV infection. The MAB810X antibody stained the same HCMV infected MRC-5 cell population as the US28-13-5G6-1D3 rAb, which was not observed for non-infected cells for either antibodies, confirming that the surface binding demonstrated for the US28-13-5G6-1D3 rAb was specific for the HCMV Ad169 infected cells.

FIG. 9. Non-specific binding of the US28-13-5G6-1D3 rAb was tested by using mouse IgG isotype control instead of the primary antibody prior to incubation with Goat anti-mouse IgG-Alexa 488. The results showed a shift in staining of the HCMV infected MRC-5 cells by the US28-13-5G6-1D3 rAb compared with the isotype control, while staining of uninfected cells (Mock) were similar to the IgG isotype control. Taken together, these results show that the US28-13-5G6-1D3 rAb binds specifically (˜16-fold greater binding) to the surface of the lytically HCMV infected cells as compared with the uninfected cells.

FIG. 10. Binding of US28-13-5G6-1D3 rAb to primary PBMCs from three HCMV seropositive individuals was investigated by using the same flowcytometry protocol as in earlier studies. The bars in the figure show the relative surface binding of the US28-13-5G6-1D3 rAb on PBMCs from the respective donors. The results showed surface binding of the US28-13-5G6-1D3 rAb on 18.37%, 3.57% and 5.25% of the total PBMCs from the three individuals, respectively. The relative proportion of PBMCs expressing certain different cell surface markers from the same individuals are listed on under the bars; totals exceed 100% because some of the same cells express more than one of these cell surface markers (however, they are not measured during the same experiment than the US28-13-5G6-1D3 rAb binding). Of the studied PBMCs, only the CD11+, CD14+ and CD16+ positive cells can be carriers of the HCMV, and these surface markers can partly overlap in different mononuclear cells. The population of mononuclear cells latently infected with HCMV often adds up to about 15% of total PBMCs (although there can be some considerable variation between individuals). Consequently, the observed surface binding of the US28-13-5G6-1D3 rAb on 18.37%, 3.57% and 5.25% of the total PBMCs in donors 1, 2 and 3, respectively, demonstrates binding to a substantial proportion of the PBMCs that can be HCMV carriers. These results are not related to the subtypes of the PBMCs, but indicate that the US28-13-5G6-1D3 rAb can bind on the surface of latently HCMV infected cells.

FIG. 11. Binding of the commercial US28 polyclonal Ab to CHO-US28-A1 cells was tested and compared with the binding of US28-13-5G6-1D3 rAb to the same cells. A. The US28-13-5G6-1D3 antibody bound to the surface of more than 50% of the CHO-US28-A1 cells (first bar from the left), while the commercial US28 Ab only bound on 10% of the cells (second bar from the left). B. We then tested the commercial US28 Ab on HCMV Ad169 infected MRC-5 cells, where it showed specific binding to the HCMV infected population. However, the commercial US28 polyclonal Ab also stained the non-infected Mock cells about two-fold compared to the IgG isotype control indicating non-specific binding on the surface of non-infected MRC-5 cells. The binding specificity of the commercial US28 Ab to HCMV Ad169 positive MRC-5 cells was only 1.7 when compared with the US28 negative Mock MRC-5 cells when the signal from IgG isotype was subtracted.

FIG. 12. US28-13-5G5-1D3 binding in 1:400 dilution (w/v) to HCMV-infected human tissues were studied by using immunohistochemistry analysis (IHC). A. Staining of the HCMV infected lung tissue with the US28-13-5G6-1D3 rAb demonstrated specificity for the HCMV infected alveolar and endothelial cells. The arrows (in the non-colour version of this figure) mark the US28-13-5G6-1D3 rAb cytoplasmic staining on typical HCMV infected alveolar cells with the characteristic cytomegalo effect in the HCMV positive control slide. B. 13-5G6-1D3 shows positive US28 staining for macrophages, but negative staining for alveolar cells in normal lung tissue. C. The same macrophages show positive HCMV DNA in the same lung biopsy, whereas there are no HCMV DNA signals in normal alveolar cells, which is coherent with the US28 staining in the same sample. Staining of the HCMV infected lung tissue with the MAB810R antibody demonstrated specificity for the HCMV infected alveolar and endothelial cells. The arrows (in the non-colour version of this figure) mark the MAB810R antibody nuclear staining on typical HCMV infected cells with the characteristic cytomegalo effect in the HCMV positive control slide. E. The IE staining is negative for the alveolar HCMV positive macrophages in normal lung tissue. The normal alveolar cells show negative staining as well. F. IgG was used as negative control antibody and showed negative staining for the same lung tissues. Colour versions of FIG. 12 are also provided alongside the non-colour versions.

FIG. 13. The HCMV US28 expression was studied by immunohistochemistry (IHC) and in situ hybridisation (ISH) in a breast cancer cohort. A. The US28-13-5G6-1D3 rAb showed strong cytoplasmic staining (3+) for the cancer cells (marked with arrows in the non-colour version of this figure) in a triple negative breast cancer (TNBC) sample. Most tumor cells in this sample stained positively for the US28-13-5G6-1D3 rAb (brown colour). B. The same sample showed some dot-like positive cytoplasmic staining in a few cells within the whole sample for the anti-IE MAB810R antibody. C. The ISH analysis shows multiple HCMV DNA signals in many nuclei of the same tumor cells, which is typical for many triple negative and HER 2 positive tumors in our material. The arrows point out some positive HCMV DNA signals in the figure. No HCMV DNA was seen in the cytoplasm of the cells. D. The negative control IgG Ab shows negative staining. A colour version of FIG. 13 is provided on the page after the non-colour version.

FIG. 14. A. Glandular metastasis from HER2 positive breast cancer shows positive intermediate cytoplasmic staining (2+) for the US28-13-5G6-1D3 rAb (brown colour) in all tumor cells. B. Most cancer cells in the same sample were negative for the MAB810R staining. Only one or two cells in the whole sample had dot-like positive, cytoplasmic staining for the MAB810R Ab (not shown in the figure). C. The ISH analysis shows many positive nuclear HCMV DNA signals n the same tumor cells. The arrows point out such positive HCMV DNA signals. The HCMV DNA was not seen in the cytoplasm of the tumor cells. D. The negative control anti-IgG Ab staining for the same sample was negative. A colour version of FIG. 14 is also provided.

FIG. 15. The US28 staining and HCMV ISH were negative in many normal adjacent tumor (NAT) breast tissues as exemplified in the figure with the sample BR1008b_J6. A. The US28-13-5G6-1D3 rAb showed negative IHC staining for the normal breast tissue. B. The MAB810R antibody showed also negative staining for the same sample. C. No nuclear or cytoplasmic HCMV DNA signals by ISH were seen for the same sample. D. The IgG staining was also negative. These results indicate that the antibodies showed consistently negative staining for the normal glandular breast tissue where there were no signs of HCMV infection. Of note, the HCMV negative sample BR1008b_J6 was placed on the same TMA slide as the samples BR1008b_D5 and BR1008b_H6, which showed strong positive staining for the US28-13-5G6-1D3 antibody and positive nuclear signals for the HCMV DNA ISH. Thus, the samples are exposed to exactly the same staining conditions and antibody/DNA probe concentrations. The positive staining seen with the US28-13-5G6-1D3 rAb was evaluated to be specific for the HCMV infected cells in both the breast cancer and normal tissues. A colour version of FIG. 15 is also provided.

FIG. 16. A. The IHC staining of the primary breast cancer samples with US28-13-5G6-1D3 rAb were specific and positive in ˜73% of the 41 studied primary breast tumor samples. 100% of the tumors that were positive for the US28 staining were also positive for the nuclear HCMV DNA signals, and there were multiple signals in the nuclei of 12/41 tumors. All 10 normal adjacent tissue controls (n=10) were negative for the cytoplasmic US28 staining in breast epithelial cells (0) and 7/10 were also negative for the ISH signals. B. The positive staining with the US28-13-5G6-1D3 rAb was seen in ˜94% of the studied 30 breast cancer metastases and was specific for the metastatic cells. All TNBC (n=7) and HER2 positive (n=11) metastases had positive US28-13-5G6-1D3 staining, and the only negative samples (n=2) were hormone receptor positive cancers. The ISH analysis showed positive nuclear HCMV DNA in 100% of the metastases. 43% of the metastatic tissues showed multiple nuclear DNA signals in the tumor cells. These samples were exclusively of TNBC or HER2+ subtype and the two exceptions had low HR+ status. The ISH results are not shown in the figure.

FIG. 17. Human glioblastoma tissues were studied with the US28-13-5G6-1D3 rAb and MAB810R antibodies by using IHC and with ISH analysis for HCMV DNA. A. The Glioblastoma grade IV brain tumor shows moderate staining for US28 in tumor cells (brown colour). B. The MAB810R antibody staining shows no IE expression in the same cancer cells. However, the positive cytoplasmic staining for MAB810R was seen in ˜90% of the glioblastoma samples. The staining was present in several cells in each positive sample which was different from the breast cancer samples. However, the staining of the glia cells was weaker than for the US28-13-5G6-1D3 rAb. C. The brain tissue NAT is negative for 13-5G6-1D3 staining. D. The ISH analysis shows positive nuclear HCMV DNA signals in the same tumor cells. The arrows indicate these signals. E. The staining of the same Gliobastoma grade IV sample is negative for the IgG antibody. F. The ISH analysis is negative for the same brain tissue NAT sample. The positive glia cells are indicated with arrows in the non-colour version of these figures. A colour version of FIG. 17 is provided on the page after the non-colour version.

FIG. 18. US28-13-5G6-1D3 staining of the human brain cancer cohort containing human astrocytoma grade 1-3 and glioblastoma grade 4 and NAT tissues. All the astrocytoma grade 1 (n=4) stained negative (0). Of the grade 2-3 astrocytomas, 6 stained negative (0), 37 had positive cytoplasmic staining (1+), and 2 had moderately positive cytoplasmic staining (2+). All of the glioblastoma grade 4 (n=19) tumors showed positive cytoplasmic staining (1+) for the US28-13-5G6-1D3 rAb, of which 3 were moderately positive (2+). The ISH analysis confirmed presence of nuclear HCMV DNA in tumor cells of all tumor samples. Only 3 of 10 NAT samples were HCMV DNA positive.

FIG. 19. A. Flowcytometry analysis with the 28-13-5G6-1D3-PE antibody were conducted on various human primary cells to exclude general off-target binding to the surface of these cell types. The following cell lines were studied: human primary adrenal cortical cells, human primary kidney epithelial cells, human primary cardiac microvascular endothelial cells, human primary liver epithelial cells and human primary vein smooth muscle cells. US28 targeting antibody 13-5G6-1D3 showed very low surface binding, far below <1% to all these cell types indicating that there is no off-target surface binding of the 13-5G6-1D3 against these cell types. These results support our observations on laboratory cells showing low binding to the surface of the normal, US28 negative cells. B. Original human primary cardiac cells were not available, and human cardiac tissue samples were therefore studied for staining of 13-5G6-1D3 by immunohistochemistry. The example in the figure shows negative 13-5G6-D3 staining of the heart muscle tissue similar to the staining with the negative IgG antibody control. The ISH analysis did not show any indications for presence of latent HCMV infection in the human heart muscle. If there were HCMV infections present in the human heart, they were always of productive infection type. The positive control was located on the same TMA plate with the heart samples and was positive: 13-5G6-1D3 showed positive staining for the malignant pheochromocytoma. The ISH control showed also typical HCMV DNA staining in the nucleus of the tumor cells in the same biopsy indicating presence of latent HCMV infection in this tumor type. The positive 13-5G6-1D3 and HCMV ISH results are marked with arrows in the figure. A colour version of FIG. 19 is also provided.

FIG. 20. Some pancreatic tissues were studied to exclude general off-target binding of the 28-13-5G6-1D3 to this tissue type. The upper row shows normal pancreatic tissue with negative staining with the 13-5G6-1D3, MAB810R and IgG control antibodies. The HCMV ISH is negative indicating absence of viral HCMV DNA in the normal pancreatic tissue sample. The lower row shows pancreatic tissue sample from another patient with positive 13-5G6-1D3 and MAB810R staining and negative IgG control antibody staining (negative control). The HCMV ISH analysis shows also positive results indicating presence of cytoplasmic HCMV DNA aggregates in the same sample. Thus, the same sample is positive for both HCMV proteins US28 and IE and HCMV DNA but negative for the negative control IgG indicating specific antibody binding. The US28 protein staining is cytoplasmic as expected, the IE protein is mostly located in the cell nucleus as expected since it is a nuclear protein. The HCMV ISH shows large cytoplasmic viral DNA aggregates since the virus is packed in the cytoplasm as indicative for productive HCMV infection. These results indicate that the antibody US28-13-5G6-1D3 does not bind off-target to pancreatic tissue and that the HCMV infection present in the morphologically normal pancreatic tissue is of productive nature in contrast to tumors where it seems to be of latent character. A colour version of FIG. 20 is also provided.

FIG. 21 A. Absolute binding levels of a panel of antibodies, comparing the binding properties of prior-art disclosed VUN100 (monovalent, or bivalent) to US28-13-5G6-1D3, with respect to control CHO cells and CHO cells expressing US28 encoded by different HCMV strains (DB and TB40/E), as well to CHO cells expressing a modified US28 containing all known US28 mutations (“mutated strain”). B. Binding specificity of the same panel of antibodies to CHO cells expressing different forms of US28 compared to control CHO cells, with results expressed as a fold-increase in binding to the US28-expressing cells compared to control CHO cells. C. Impact of strain differences on the extent of absolute binding levels of the same panel of antibodies, shown as a percentage change in absolute binding levels between the binding to CHO cells expressing US28 encoded by HCMV strains DB and TB40/E, wherein a smaller percentage change is indicative of a greater degree of strain agnostic binding. D. Percentage retention of binding specificity of the same panel of antibodies, between CHO cells expressing the forms of US28 encoded by HCMV strains DB and TB40/E, wherein a retention of binding specificity close to 100% is indicative of strain agnostic binding specificity, whereas a more substantial difference (as demonstrated by the two VUN100 antibody samples) indicates a change in binding specificity dependent on the strain that encodes US28 (i.e. a lack of strain agnostic binding specificity). E. Percentage binding of antibodies to CHO cells expressing the different forms of US28 (as encoded by DB, TB40/E and the mutated form), when normalised to the level of binding observed for 1D3 for each US28 form, wherein changes in the % binding for a given antibody, across the different CHO forms, is indicative of a reduction in strain agnostic binding activity compared to the 1D3-mFc antibody, and shows that binding more dependent on the strain that encodes US28 (i.e. a lack of strain agnostic binding compared to the 1D3-mFc antibody).

FIG. 22. Percentage off-target binding of various ECD3-binding molecules of the invention (A) or various SEQ ID NO: 177-binding molecules of the invention (B), in comparison with VUN100, as assessed by binding to control CHO cells.

FIG. 23. Fold change in binding specificity for exemplary ECD3-binding molecules binding to US28-expressing CHO versus control CHO cells, relative to the level of specificity of VUN100 in the same assay. A. Improved binding specificity for 1D3 compared with VUN100 (n=3). B. Improved binding specificity for 1C10 compared with VUN100 (n:=3). C. Improved binding specificity for 1A10 compared with VUN100 (n=3). D. Improved binding specificity for 1G4 compared with VUN100 (n=3). E. Improved binding specificity for 1E8 compared with VUN100 (n=1).

FIG. 24. Fold change in binding specificity for exemplary SEQ ID NO:177-binding molecules binding to US28-expressing CHO versus control CHO cells, relative to the level of specificity of VUN100 in the same assay.

FIG. 25. The binding of the N-terminal antibodies to HCMV infected lung tissue and HCMV negative myometrium were studied by IHC. A. Previously exemplified US28-13-5G6-1D3 rAb was used as positive control and showed optimal IHC staining in these studies in 1:3200 (w/v) dilution. B. The US28-4-2F5B11 rAb (1:1600 dilution (w/v)) all showed positive staining on HCMV infected cells and negative staining for HCMV negative myometrium. C. The US28-4-4F3C3 rAb (1:800 dilution (w/v)) showed HCMV specific staining for infected lung tissue and negative staining for the HCMV negative myometrium. D. The US28-4-7B1F3 rAb (1:6400 dilution (w/v)) showed positive and HCMV specific staining in HCMV positive lung tissue but not in the myometrium. E. Mouse lung tissue with staining IgG was used as negative control antibody. The staining of the HCMV infected lung tissue with the exemplified ECD1-binding molecules demonstrated specificity for HCMV infected alveolar cells as there was no staining of the myometrial cells and fibroblasts in the corresponding HCMV negative myometrium controls. As with the US28-13-5G6-1D3 rAb, the ECD1-binding molecules stained typical HCMV infected cells with the characteristic cytomegalo effect, showing strong cytoplasmic or membranous staining in these cells, as the US28 protein is known to be located in cytoplasm and cell membrane, and stained positively for some tissue macrophages, which are known to be carriers of latent or reactivated HCMV.

FIG. 26. Z Score distribution of Sample hum7B1F3 (0.1 μg/m) on a native HuProt array, showing the top 50 ranked hits.

FIG. 27. Z Score distribution of Sample mouse 7B1F3 (0.1 μg/ml) on a native HuProt array, showing the top 50 ranked hits.

FIG. 28. Z Score distribution of Sample hum7B1F3 (0.1 μg/ml) on a native HuProt array, showing the top hit and selected additional hits from the HuProt array.

FIG. 29. Z Score distribution of Sample mouse 7B1F3 (0.1 μg/ml) on a native HuProt array, showing the top hit and selected additional hits from the HuProt array.

FIG. 30. Structural analysis of 7B1F3. The V_{H} is positioned on the left, comprised of the indicated CDR-H1, CDR-H2 and CDR-H3; and the V_{L} is positioned on the right comprised of the indicated CDR-L1, CDR-L2 and CDR-L3. The N-glycosylation sequence liability is indicated by a thatched circle.

FIG. 31. A comparison of the binding between parental murine 7B1F3 (“7B1F3”) and humanised 7B1F3 (“hum7B1F3”).

FIG. 32. A comparison of the binding between parental murine 7B1F3 (“7B1F3”) and humanised, aglycosylated 7B1F3 (“hum7B1F3”) at 0.5 μg/mL dose.

FIG. 33. Variation in relative binding to US28 derived from different HCMV strains (VHL/E, DB, TB40/E and Merlin) for three exemplary ECD1-binding molecules of the present invention (antibodies 2F5B11, 4H3C3 and 7B1F3) and the prior art antibody bivalent VUN100. Data for each antibody clone are assessed against the binding to US28 protein as encoded by the VHL/E strain of HCMV, as discussed further in Example 8.

FIG. 34. Typical assay timeline for Example 9.

FIG. 35. Heat map of Tumor cell death: E-max (IC50 max)/E-Control (IC50 control) in each 3D PDX tumor model.

FIG. 36. Tumour size results (1 μM Staurosporine is the positive control).

FIG. 37. Tumour size IC50 curves.

FIG. 38. Exemplary configurations of bispecific antibodies using the 7B1F3 binding domain.

### DETAILED DESCRIPTION OF THE INVENTION

The present invention relates to agents targeting a specific region, or regions, of the US28 protein, as encoded by human cytomegalovirus (HCMV), and therapeutic, prophylactic and diagnostic approaches related thereto including but not limited to HCMV-infected cancers and other conditions associated with latent or lytic HCMV infections.

HCMV US28 protein represents excellent potential for targeting HCMV infections, including the latent reservoir, because:
(1) Certain parts of the US28 protein are expressed on the cell surface during both lytic and latent HCMV infections (Elder et al., iScience, 2019, 12: 13-26);
(2) US28 is a GCPR, of which trafficking to the plasma membrane allows both its direct targeting with binding molecules and its use as a transporter of payload due to its endocytosis, which is either constitutive or occurs as a result of ligand binding. GCPRs in general constitute the largest family of proteins targeted by approved drugs (Srirarn & Insel, Mol Pharmacol, 2018, 93(4): 251-258); and
(3) In contrast to many approved drugs, which target the GCPRs encoded by human DNA, the HCMV US28 is entirely encoded by viral DNA, employing therefore a highly specific drug target exclusively located in the HCMV infected but not in healthy human cells.

### A. Binding Molecules to US28

When considering the design of binding molecules against US28, the inventor noted that the N-terminal domain is the ligand binding part of the US28 protein, physically extending from the plasma membrane and therefore, most likely exposed to host antibody production, immune response, and genetic selection pressure (Mozzi et al, 2020, supra). Consistently, it is also an area known to contain high inter-strain variability and mutations (Arav-Boger et al., 2002, supra). The less conserved sites, which the inventor has identified in particular within the N-terminal region to be consistently in the region comprising amino acid positions 1 to 25 of the N-terminal region, are also more susceptible to develop new mutations, which may change the response to treatment, that targets these areas over time (Komatsu et al., Antiviral Res, 2014, 101: 12-25). Numerous such N-terminal polymorphism within the region comprising amino acid positions 1 to 25 are known within identified HCMV strains, as summarised in Table 3 of the present application. Thus, new mutations in HCMV genes are likely to arise in such less conserved sites, which are pinpointed by the variations between different viral strains. Most structural antibodies arise most likely against the N-terminal part of the US28 protein (De Groof et al., Mol Pharm, 2019, 16(7): 3145-3156), which may therefore also be a case for the natural antibodies in human body (Elder et al, 2019, supra).

According to our knowledge, three types of HCMV high-risk oncogenic strains have so far been identified. The DB (KT95923) and EL (MW980585) clinical HCMV isolates have been recently identified to promote oncogenic molecular pathways, establish anchorage-independent growth in vitro and produce tumorigenicity in mice models, and are therefore named as high-risk oncogenic strains (Kumar et al., 2018 and Ahmad et al., 2021). In addition, Soroceanu et al. sequenced the C-terminal part of the US28 gene in 10 HCMV positive glioblastoma tumors. Alignment of the results (presented in NIHMS323374-supplement-1.pdf for the original publication) showed that all of these strains would be similar to the HCMV VHL/E clinical isolate (L20501.1). The high-risk oncogenic strains DB, BL and VHL/E all show different mutations in the US28 gene, especially in the N-terminal part (Table 3). The N-terminal part differ from DB strain in positions E18D; A19E; F25L (VHL/E) and A19D, T21A, F25L (BL). Thus, the amino acid positions 18, 19, 21 and 25 are all mutated in these high-risk oncogenic strains; and other strains are also known to have mutations at positions 8 and/or 15 (Table 3).

As a consequence of the high levels of polymorphism reported in the N-terminal region, and prior to the present invention, it appeared that this region of US28 may be a sub-optimal target for designing anti-US28 antibodies, and other anti-US28 binding molecules, in particular since the binding activity against US28 may then depend substantially on the strain of HCMV present being a strain that presents an N-terminal that matches with said binding activity. Furthermore, since it is apparent that genetic drift that can occur during HCMV infections, such drift may be concentrated in highly polymorphic areas, such that an HCMV infection may evolve during the course of infection to avoid binding sensitivity to anti-US28 antibodies, and other anti-US28 binding molecules, targeted against such N-terminal sequences.

Indeed, as already discussed in the Background section of the present application, a previously-described anti-N-terminal VHH antibody termed VUN100 was shown to bind to a discontinuous epitope, which comprise multiple binding positions in the N-terminal extracellular region of US28, and further influenced by the presence of the third extracellular loop (also referred to herein as ECD4) of US28, as discussed in Example 3 of WO 2019/151865 (page 36, lines 11-32) and the legend to FIG. 2 of De Groof et al, 2019 (supra).

It was notable that the results in relation to the binding of VUN100 to the different HCMV strains in FIG. 8D of WO 2019/151865 show a difference in binding between the VHL/E, Merlin and TB40/E strains of HCMV, with binding being particularly reduced in strain TB40/E (81 type) at around only half the level of binding observed against the Merlin strain. Moreover, further characterisation of VUN100 is reported in a pre-printed article available online by De Groof et al, 2020 (doi: https://doi.org/10.1101/2020.05.12.071860), wherein FIG. 2 of the supplementary data gives the results of the % induced IE expression in the nucleus of CD14+ monocytes bound by VUN100. All cells tested were from HCMV seropositive individuals, and confirmed to be latently infected with HCMV, although the strain(s) of HCMV infecting each donor were undetermined. The level of IE expression induced by VUN100 binding to these HCMV-positive CD14+ cells from each of the four different patients varied substantially, with the reported figures being 57%, 33%, 22% and 4% (a range of difference of greater than 14-fold), respectively for cells from donors 1-4. This high level of response variability following the binding of VUN100 to the confirmed HCMV-positive cells seems to be most likely due to the infection of each of the donors with different HCMV strains, and thus a strong indication that the binding ability of VUN100 will vary considerably between different strains of HCMV. The same figure also shows high levels of response variability (in excess of 7-fold levels of difference) following the binding of a bivalent form of VUN100 (termed VUN100b by De Groof et at, 2020 (supra) as represented by SEQ ID NO: 63 of the present application) to the same group of HCMV-positive cells from the donors 1-4, again providing results indicative of strain-specific binding sensitivities. De Groof et al, 2021, Pharmacol Rev 73:828-846, also describes how serial passage of HCMV results in the development of resistant mutants with a truncated US28, having a premature stop codon in the extracellular loop 3, that results in reduced surface expression of US28. Such resistance may have adverse implications for VUN100, which relies on a partial epitope in the third extracellular loop.

Despite its highly variable sequences, the inventor considered whether a specific region of the N-terminal part of the US28 protein (ECD1) existed that could be an appropriate target for a highly specific, and strain agnostic, antibody against US28.

The inventor also determined that ECD2 and ECD3 of US28 are highly conserved between the different HCMV strains; ECD2 does not have any known mutations, whereas the inventor's analysis of the known sequences of the ECD3 from many different HCMV strains revealed the existence of only one known mutation. Of the known high-risk oncogenic strains, the DB strain is ECD3 N170N (also referred to herein as the “4N” variant form, as position 170 of the US28 protein corresponds to position 4 of ECD3), whereas the BL and VHL/E strains represent the N170D (also referred to herein as the ‘4D’) variants (Table 3). The inventor identified that DNA sequence encoding US28 ECD4 DNA contains multiple polymorphic sites, of which only one leads to a more common amino acid change (R267K). Of note, the high-risk oncogenic BL strain contains both ECD4 variants V250L and R267K (Table 3).

Surprisingly, the present studies demonstrated that ECD2 and the conserved parts of ECD4 protein were not appropriate immunogens in mice, whereas the applicant was able to develop a new approach to the identification of highly specific, strain agnostic, binding molecules against a particular region of ECD1 of HCMV US28 corresponding to positions 26 to 37 and comprising the sequence of TDVLNQSKPVTL (SEQ ID NO: 177), which the inventor's analysis determined to be apparently conserved in all known strains of HCMV, and also highly specific, strain agnostic, binding molecules against the ECD3 regions of HCMV US28. As reported in the examples of the present application, a number of monoclonal antibodies were shown to bind well and specifically to a peptide representing this conserved epitope within ECD1, on US28 overexpressing US28-CHO-A1 cells, and HCMV infected human lung tissue. Furthermore, ECD3 monoclonal antibodies were shown to bind well and specifically on both genetic variants of the US28 ECD3 peptides, on US28 overexpressing US28-CHO-A1 cells, HCMV Ad169 infected MRC-5 cells, primary PBMCs from HCMV seropositive individuals, HCMV infected human lung tissue and several types of aggressive human tumors, such as oesophagus, gastric, rectum, liver, lung, pancreas, cervical cancers, malignant pheochromocytoma and locally advanced colon cancer, breast cancer and its metastasis and glioblastoma grade 4.

Accordingly, the applicant has provided highly specific ECD1-binding molecules against the US28 protein encoded by HCMV, which have the potential to provide HCMV strain agnostic binding (and/or can be adapted in accordance with the teachings of the present application to further include an ECD3-binding domain that is highly specific and HCMV strain agnostic), including, but not limited to, in particular, antibodies and chimeric antigen receptors (‘CARs’), and uses thereof, for example in diagnostic, prophylactic and therapeutic uses and methods related to HCMV. Also provided are HCMV vaccines and other agents suitable for use in generating said binding molecules.

A first aspect of the present invention provides a binding molecule, comprising one or more polypeptide chains, said binding molecule having binding specificity to a first epitope within a polypeptide consisting of the amino acid sequence of TDVLNQSKPVTL (SEQ ID NO: 177) within the ECD1 of a US28 protein of human cytomegalovirus (HCMV), and optionally, wherein the binding molecule also has binding specificity to a second epitope within extracellular domain 3 (ECD3) of a US28 protein of HCMV (and/or wherein said binding molecule is a first binding molecule that is formulated with a second binding molecule that has binding specificity to a second epitope within ECD3 of a US28 protein of HCMV), wherein ECD1, SEQ ID NO: 177, and ECD3, of the US28 protein comprises an amino acid sequence presented in the US28 protein at positions corresponding to positions 1 to 37, 26 to 37, and 167 to 183, respectively, of the US28 protein encoded by human cytomegalovirus (HCMV) as set forth in SEQ ID NO: 5. Non-limiting, but particularly preferred, examples of said binding molecule according to the first aspect of the present invention includes antibodies and chimeric antigen receptors (CARs), as discussed further below.

### Epitopes:

As noted above, binding molecules according to the first aspect of the present invention have binding specificity to a first epitope within a polypeptide consisting of the amino acid sequence of TDVLNQSKPVTL. (SEQ ID NO: 177) within the ECD1 of a US28 protein of human cytomegalovirus (HCMV), and optionally, wherein the binding molecule also has binding specificity to a second epitope within extracellular domain 3 (ECD3) of a US28 protein of HCMV (and/or wherein said binding molecule is a first binding molecule that is formulated with a second binding molecule that has binding specificity to a second epitope within ECD3 of a US28 protein of HCMV), wherein ECD1, SEQ ID NO: 177 and ECD3 of the US28 protein comprises an amino acid sequence presented in the US28 protein at positions corresponding to positions 1 to 37, 26 to 37 and 167 to 183, respectively, of the US28 protein encoded by human cytomegalovirus (HCMV) as set forth in SEQ ID NO: 5.

The first epitope to which the binding molecule of the first aspect of the present invention has binding specificity may, for example, preferably be present entirely within a polypeptide consisting of the amino acid sequence of TDVLNQSKPVTL (SEQ ID NO: 177) within ECD1 of the US28 protein of HCMV. The optional second epitope to which the binding molecule of the first aspect of the present invention has binding specificity may, for example, preferably be present entirely within ECD3 of the US28 protein of HCMV.

The, or each, first and second epitope may, for example, be a linear epitope within (preferably entirely within) a polypeptide consisting of the amino acid sequence of TDVLNQSKPVTL (SEQ ID NO: 177) within ECD1 and/or ECD3, respectively, of the US28 protein. Alternatively, the or each, first and second epitope may be a discontinuous and/or conformational epitope within (preferably entirely within) a polypeptide consisting of the amino acid sequence of TDVLNQSKPVTL. (SEQ ID NO: 177) within ECD1 and/or ECD3, respectively, of the US28 protein.

The first epitope may, for example, comprise or consist of 12 or fewer amino acids of the sequence of SEQ ID NO: 177, for example it may comprise or consist of 11, 10, 9, 8, 7, 6, 5, 4, 3 or fewer amino acids of the sequence of SEQ ID NO: 177, which may optionally be consecutive amino acids within the sequence of SEQ ID NO: 177 within ECD1 of US28.

The optional second epitope may, for example, comprise or consist of 17 or fewer amino acids of the ECD3 of the US28 protein of HCMV, for example it may comprise or consist of 16, 15, 14, 13, 12, 11, 10, 9, 8, 7, 6, 5, 4, 3 or fewer amino acids of the ECD3 of the US28 protein of HCMV, which may optionally be consecutive amino acids in ECD3 of US28.

The amino acid sequence presented in the US28 protein at positions corresponding to positions 1 to 37 (ECD1) and/or 167 to 183 (ECD3) of the US28 protein encoded by human cytomegalovirus (HCMV) as set forth in SEQ ID NO: 5 may not necessarily be identical to the sequence found in that region of SEQ ID NO: 5, since there may be inter-strain variation in the sequence of ECD1 and/or ECD3 of US28 amongst different HCMV strains. Accordingly, in this context, the term “corresponding to” refers to the amino acids found in the region that has a corresponding position (that is, in the: (i) 1′ extracellular loop, also referred to interchangeably as the ECD1 region and N-terminus; and/or (ii) 2^{nd} extracellular loop, also referred to as the ECD3 region; respectively) of the US28 protein encoded by any HCMV strain of interest.

Following an analysis of the sequence of the US28 protein from many different clinical and lab strains of HCMV, the applicant has identified the presence of a conserved region within the N-terminus (ECD1) of US28 corresponding to SEQ ID NO: 177 apparently within all known HCMV strains, and only a single polymorphism within ECD3 of HCMV-encoded US28, thus presenting a favourable ECD1 sequence, and two alternative ECD3 sequences. The two alternative ECD3 sequences are referred to herein as the 4D-variant and the 4N-variant.

The favourable ECD1 sequence of US28 as defined by SEQ ID NO: 177 is conserved apparently across all known HCMV strains, including but not limited to the HCMV strains DB, Towne, AD169, BL, DAVIS, JP, Merlin, PH, TB40/E, Toledo, TR, VHL/E and VR1814 (FIX).

The 4D-variant refers to a sequence variation present in ECD3 of US28, as encoded by a first group of HCMV strains, and appears to be the more common form; around 90% of the sequences of US28 encoded by different HCMV strains, as identified by a BLAST search, show the 4D-variant sequence. The 4D-variant is characterised by comprising the sequence of TKKDNQCMTDYDYLEVS (SEQ ID NO: 7; position 4 of which, as underlined, is D) in ECD3 of US28. Exemplary HCMV strains of the first group, having the 4D-variant of US28 include the Towne, VR1814, TB40/E, VHL/E, Merlin, JP, Ad169, AF1, BL and DAVIS strains.

The 4N-variant refers to an alternate sequence variation present in ECD3 of US28, as encoded by a second group of HCMV strains, and appears to be the less common form. The 4D-variant is characterised by comprising the sequence of TKKNNQCMTDYDYLEVS (SEQ ID NO: 6; position 4 of which, as underlined, is N) in ECD3 of US28. Exemplary HCMV strains of the second group, having the 4N-variant of US28 include the Toledo, TR and DB strains. These, and other strains of HCMV showing the 4N-variant sequence in ECD3 of US28 are shown the Table 1.

The first epitope to which the binding molecule of the first aspect of the present invention, such as a HMCV strain agnostic binding molecule of the first aspect of the present invention, has binding specificity is an epitope that is present within the sequence TDVLNQSKPVTL (SEQ ID NO: 177). To put it another way, the epitope within ECD1 of US28 to which the binding molecule of the first aspect of the present invention has binding specificity, preferably excludes the amino acids at positions 1 to 25 of the N-terminal region of US28.

In a further option, wherein the first epitope is a discontinuous and/or conformational epitope within (preferably entirely within) the sequence TDVLNQSKPVTL (SEQ ID NO: 177), then the discontinuous epitope may include any 3, 4, 5, 6, 7, 8, 9, 10 or 11 amino acids of the sequence of SEQ ID NO: 177.

A linear, discontinuous or conformational first epitope within the sequence TDVLNQSKPVTL (SEQ ID NO: 177) may exclude one or more of amino acids of SEQ ID NO: 177, which comprises a 1^{st} through to a 11^{th} position, within the sequence that corresponds to TDVLNQSKPVTL.

Additionally, or alternatively, one or more of the amino acids of TDVLNQSKPVTL may be excluded in the linear, discontinuous or conformational first epitope that is bound by binding molecules of the present invention, such as HMCV strain agnostic binding molecules of the first aspect of the present invention.

For example, a linear, discontinuous or conformational epitope within SEQ ID NO: 177 may additionally or alternatively exclude the 1^{st} position. A linear, discontinuous or conformational epitope within SEQ ID NO: 177 may additionally or alternatively exclude the 2^{nd} position. A linear, discontinuous or conformational epitope within SEQ ID NO: 177 may additionally or alternatively exclude the 3^{rd} position. A linear, discontinuous or conformational epitope within SEQ ID NO: 177 may additionally or alternatively exclude the 4^{th} position. A linear, discontinuous or conformational epitope within SEQ ID NO: 177 may additionally or alternatively exclude the 5^{th} position. A linear, discontinuous or conformational epitope within SEQ ID NO: 177 may additionally or alternatively exclude the 6^{th} position. A linear, discontinuous or conformational epitope within SEQ ID NO: 177 may additionally or alternatively exclude the 7^{th} position. A linear, discontinuous or conformational epitope within SEQ ID NO: 177 may additionally or alternatively exclude the 8^{th} position. A linear, discontinuous or conformational epitope within SEQ ID NO: 177 may additionally or alternatively exclude the 9^{th} position. A linear, discontinuous or conformational epitope within SEQ ID NO: 177 may additionally or alternatively exclude the 10^{th} position. A linear, discontinuous or conformational epitope within SEQ ID NO: 177 may additionally or alternatively exclude the 11^{th} position. A linear, discontinuous or conformational epitope within ECD3 may additionally or alternatively exclude the 12^{th} position.

The first epitope to which the binding molecule of the first aspect of the present invention, such as a HMCV strain agnostic binding molecule of the first aspect of the present invention, has binding specificity is preferably an epitope that is common to, and present within, greater than 90% of the known clinical and/or lab strains of HCMV (including, at least, all of the HCMV strains disclosed in the present application), for example at least 91%, 92%, 93%, 94%, 95%/a, 96%, 97%, 98%, 99% or substantially 100% (for example, between 90% and 100%, between 91% and 100%, between 92% and 100%, between 93% and 100%, between 95% and 100%, between 96% and 100%, between 97% and 100%, between 98% and 100%, between 99 and 100%) of the clinical and/or lab strains of HCMV (including, at least, all of the HCMV strains disclosed in the present application).

In one preferred embodiment, the amino acids in the first epitope to which the binding molecule of the first aspect of the present invention has binding specificity may include, or be identical to, the amino acids in the epitope within ECD1 that is bound by any one or more of the 4H3C3, 7B1F3 and/or 2F5B11 antibodies as described herein (by which is included also an scFv comprising a V_{H} polypeptide sequence having the V_{H} sequence of 4H3C3, 7B1F3 and/or 2F5B11 as defined by SEQ ID NOs: 187, 211, 233, respectively, and a V_{L} polypeptide sequence having the V_{L} sequence of 4H3C3, 7B1F3 and/or 2F5B11, as defined by SEQ ID NOs: 189, 213 and 235, respectively), or vary from said epitope by not more than 5, 4, 3, 2 or 1 amino acids.

The second epitope to which the binding molecule of the first aspect of the present invention, such as a HMCV strain agnostic binding molecule of the first aspect of the present invention, optionally also has a further binding specificity is preferably an epitope that is common to, and present within, both the sequence TKKNNQCMTDYDYLEVS (SEQ ID NO: 6, corresponding to the 4N variant of ECD3 of US28) and the sequence of TKKDNQCMTDYDYLEVS (SEQ ID NO: 7, corresponding to the 4D variant of ECD3). To put it another way, the epitope within ECD3 of US28 to which the binding molecule of the first aspect of the present invention optionally also has binding specificity, preferably excludes the 4^{th} amino acid residue of each of SEQ ID NOs: 6 and 7, which corresponds to N in the 4N-variant and D in the 4D-variant.

In the case of linear ECD3 epitopes, then optionally, epitopes which are common to, and present within, both of the 4N- and 4D-variants, and which exclude the variant 4^{th} amino acid residue, could include all 13 amino acids of the sequence NQCMTDYDYLEVS, or any 12, 11, 10, 9, 8, 7, 6, 5, 4, 3 or fewer amino acids thereof, which may optionally be consecutive amino acids of said sequence.

In a further option, wherein the second epitope is a discontinuous and/or conformational epitope within (preferably entirely within) ECD3 of the US28 protein, then the discontinuous epitope may include any 3, 4, 5, 6, 7, 8, 9, 10, 11, 12, 13, 14, 15 or 16 of the 17 amino acids of the 4N variant ECD3 sequence of TKKNNQCMTDYDYLEVS and/or of the 4D variant ECD3 sequence of TKKDNQCMTDYDYLEVS.

A linear, discontinuous or conformational second epitope within ECD3 may exclude one or more of amino acids of ECD3 of US28, which comprises a 1^{st} to through to a 17^{th} position, within the sequence that corresponds to TKKNNQCMTDYDYLEVS in the 4N variant and TKKDNQCMTDYDYLEVS in the 4D variant.

In one preferred embodiment, a linear, discontinuous or conformational second epitope within ECD3 preferably excludes the ₄th position (N/D) which is known to vary between different HCMV strains.

Additionally, or alternatively, one or more of the amino acids of ECD3 of US28 may be excluded in the linear, discontinuous or conformational second epitope that is optionally bound by binding molecules of the present invention, such as HMCV strain agnostic binding molecules of the first aspect of the present invention.

For example, a linear, discontinuous or conformational epitope within ECD3 may additionally or alternatively exclude the 1^{st} position. A linear, discontinuous or conformational epitope within ECD3 may additionally or alternatively exclude the 2^{nd} position. A linear, discontinuous or conformational epitope within ECD3 may additionally or alternatively exclude the 3^{rd} position. A linear, discontinuous or conformational epitope within ECD3 may additionally or alternatively exclude the 5^{th} position. A linear, discontinuous or conformational epitope within ECD3 may additionally or alternatively exclude the 6^{th} position. A linear, discontinuous or conformational epitope within ECD3 may additionally or alternatively exclude the 7^{th} position. A linear, discontinuous or conformational epitope within ECD3 may additionally or alternatively exclude the 8^{th} position. A linear, discontinuous or conformational epitope within ECD3 may additionally or alternatively exclude the 9^{th} position. A linear, discontinuous or conformational epitope within ECD3 may additionally or alternatively exclude the 10^{th} position. A linear, discontinuous or conformational epitope within ECD3 may additionally or alternatively exclude the 11^{th} position. A linear, discontinuous or conformational epitope within ECD3 may additionally or alternatively exclude the 12^{th} position. A linear, discontinuous or conformational epitope within ECD3 may additionally or alternatively exclude the 13^{th} position. A linear, discontinuous or conformational epitope within ECD3 may additionally or alternatively exclude the 14^{th} position. A linear, discontinuous or conformational epitope within ECD3 may additionally or alternatively exclude the 15^{th} position. A linear, discontinuous or conformational epitope within ECD3 may additionally or alternatively exclude the 16^{th} position. A linear, discontinuous or conformational epitope within ECD3 may additionally or alternatively exclude the 17^{th} position.

The second epitope to which the binding molecule of the first aspect of the present invention, such as a HMCV strain agnostic binding molecule of the first aspect of the present invention, optionally also has binding specificity is preferably an epitope that is common to, and present within, greater than 90% of the known clinical and/or lab strains of HCMV (including, at least, all of the HCMV strains disclosed in the present application), for example at least 91%, 92%, 93%, 94%, 95%, 96%, 97%, 98%, 99% or substantially 100% (for example, between 90% and 100%, between 91% and 100%, between 92% and 100%, between 93% and 100%, between 95% and 100%, between 96% and 100%, between 97% and 100%, between 98% and 100%, between 99 and 100%) of the clinical and/or lab strains of HCMV (including, at least, all of the HCMV strains disclosed in the present application).

In one preferred embodiment, the amino acids in the second epitope to which the binding molecule of the first aspect of the present invention can optionally have binding specificity may include, or be identical to, the amino acids in the epitope within ECD3 that is bound by any one or more of the US28-13-5G6-1D3, 13-1C10-1C10, 13-1H3-1A10, 13-1C10-1G9, 14-4E4-1E8 antibodies (commonly abbreviated herein to “1D3”, “1C10”, “1A10”, “1G9” and “1E8”, respectively) as described herein (by which is included also an scFv comprising a V_{H} polypeptide sequence having the V_{H} sequence of 1D3, 1C10, 1A10, 1G9 and/or 1E8 as defined by SEQ ID Nos: 12, 104, 122, 68 and 88, respectively, and a V_{L} polypeptide sequence having the V_{L} sequence of 1D3, 1C10, 1A10, 1G9 and/or 1E8 as defined by SEQ ID Nos: 18, 108, 126, 72 and 92, respectively), or vary from said epitope by not more than 5, 4, 3, 2 or 1 amino acids.

### Binding Properties:

As reported in the examples of the present application, multiple monoclonal antibodies have been generated against a polypeptide consisting of the amino acid sequence of TDVLNQSKPVTL (SEQ ID NO: 177) within ECD1 of US28, by using the methods described herein, and were shown to bind well and specifically to a peptide representing the conserved epitope within ECD1, on US28 overexpressing US28-CHO-A1 cells, and HCMV infected human lung tissue.

Furthermore, multiple monoclonal antibodies have been generated against ECD3 of US28, by using the methods described herein, and were shown to bind well and specifically on both genetic variants of the US28 ECD3 peptides and on US28 overexpressing US28-CHO-A1 cells, and certain exemplary antibodies therefrom were further tested and shown to provide excellent binding properties to HCMV Ad169 infected MRC-5 cells, primary PBMCs from HCMV seropositive individuals, HCMV infected human lung tissue and several types of aggressive human tumors, such as oesophagus, gastric, rectum, liver, lung, pancreas, cervical cancers, malignant pheochromocytoma and locally advanced colon cancer, breast cancer and its metastasis and glioblastoma grade 4.

These monoclonal antibodies are exemplary embodiments of ECD1- and ECD3-binding molecules according to the present invention, which could be combined in a multispecific format.

Various assays are described herein that can be used to assess the properties of binding molecules according to the first aspect of the present invention. In these assays, a “reference binding molecule” is specified as a comparison, which is in reference to particular reference binding molecules demonstrated herein for ECD1 or ECD3. Where a binding molecule according to the first aspect of the present invention is capable of binding to an epitope within a polypeptide consisting of the amino acid sequence of TDVLNQSKPVTL (SEQ ID NO: 177) in ECD1, the reference binding molecule will be an ECD1-reference binding molecule, such as 4H3C3, 7B1F3, 2F5B1.1, 4A11A11, 2G2B3, 5D6H11, 9G3B9, 5E8F7, 6G3D6 and/or 5E1E4. Where a binding molecule according to the first aspect of the present invention is also capable of binding to an epitope within ECD3, the reference binding molecule will be an ECD3-reference binding molecule for assessing the ECD3-binding, such as 1D3, 1C10, 1A10, 1G4 and/or 1E8. Accordingly, a multispecific binding molecule may have its ECD1-binding domain assessed against an ECD1 reference binding molecule, and its ECD3-binding domain assessed against an ECD3 reference binding molecule.

More specifically, for ECD1, the 4H3C3, 7B1F3 and/or 2F5B11 antibodies as described herein (by which is included also an scFv comprising a V_{H} polypeptide sequence having the V_{H} sequence of 4H3C3, 7B1F3 and/or 2F5B11 as defined by SEQ ID NOs: 187, 211 and 233, respectively, and a V_{L} polypeptide sequence having the V_{L} sequence of 4H3C3, 7B1F3 and/or 2F5B11, as defined by SEQ ID NOs: 189, 213 and 235, respectively) are preferred exemplary binding molecules according to the present invention, although other binding molecules have been produced and the scope of the first aspect of the present invention is not limited only to 4H3C3, 7B1F3 and/or 2F5B11, nor only to binding molecules derived therefrom (such as other binding molecules sharing the CDRs of 4H3C3, 7B1F3 and/or 2F5B11), although these may represent various preferred embodiments. Nevertheless, the 4H3C3, 7B1F3 and/or 2F5B11 antibodies (by which is included also an scFv comprising a V_{H} polypeptide sequence having the V_{H} sequence of 4H3C3, 7B1F3 and/or 2F5B11 as defined by SEQ ID NOs: 187, 211 and 233, respectively, and a V_{L} polypeptide sequence having the V_{L} sequence of 4H3C3, 7B1F3 and/or 2F5B11 as defined by SEQ ID NOs: 189, 213, 235, respectively) can provide a useful benchmark against which to characterise the binding properties of other binding molecules according to the first aspect of the present invention.

Furthermore, for ECD3, the 1D3, 1C10, 1A10, 1G9 and/or IE8 antibodies as described herein (by which is included also an scFv comprising a V_{H} polypeptide sequence having the V_{H} sequence of 1D3, 1C10, 1A10, 1_G9 and/or 1E8 as defined by SEQ ID Nos: 12, 104, 122, 68 and 88, respectively, and a V_{L} polypeptide sequence having the V_{L} sequence of 1D3, 1C10, 1A10, 1G9 and 1E8 as defined by SEQ ID Nos: 18, 108, 126, 72 and 92, respectively) are preferred exemplary binding molecules according to the present invention, although other binding molecules have been produced and the scope of the first aspect of the present invention is not limited only to 1D3, 1C10, 1A10, 1G9 and/or 1E8, nor only to binding molecules derived therefrom (such as other binding molecules sharing the CDRs of 1D3, 1C10, 1A10, 1G9 and/or 1E8), although these may represent various preferred embodiments. Nevertheless, the 1D3, 1C10, 1A10, 1(39 and/or 1E8 antibodies (by which is included also an scFv comprising a V_{H} polypeptide sequence having the V_{H} sequence of 1D3, 1C10, 1A10, 1.G9 and/or IE8 as defined by SEQ ID Nos: 12, 104, 122, 68 and 88, respectively, and a V_{L} polypeptide sequence having the V_{L} sequence of 1D3, 1C10, 1A10, 1G9 and/or 1E8 as defined by SEQ ID Nos: 18, 108, 126, 72 and 92, respectively) can provide a useful benchmark against which to characterise the binding properties of other binding molecules according to the first aspect of the present invention that further include specificity for ECD3.

For example, a binding molecule according to the first aspect of the present invention, having binding specificity to a first epitope within a polypeptide consisting of the amino acid sequence of TDVLNQSKPVTL (SEQ ID NO: 177) within ECD1 (and optionally also having binding specificity to a second epitope within ECD3) of a US28 protein, may demonstrate binding properties that are similar or substantially equivalent to the binding properties of any one, or more, of 4H3C3, 7B1F3, 2F5B11, 4A11A11, 2G2B3, 5D6H11, 9G339, 5E8F7, 6G3D6 and/or 5E1E4, when tested under the same conditions as 4H3C3, 7B1F3, 2F5B11, 4A11A11, 2G263, 5D6H11, 9G3B9, 5E8F7, 6G3D6 and/or 5E1E4, respectively (and optionally 1D3, 1C10, 1A10, 1G4, 1G9 and/or 1E8, when tested under the same conditions as 1D3, 1C10, 1A10, 1G4, 1G9 and/or 1E8, respectively).

In multispecific formats, wherein the binding molecule has specificity both for a polypeptide consisting of the amino acid sequence of TDVLNQSKPVTL (SEQ ID NO: 177) within ECD1, and for ECD3, a binding molecule according to the first aspect of the present invention may demonstrate ECD1 binding properties that are similar or substantially equivalent to the binding properties of an appropriate ECD1-binding molecule as described herein (such as any one, or more, of 4H3C3, 7B1F3, 2F5B11, 4A11A11, 2G2B3, 5D6H11, 9G3B9, 5E8F7, 6G3D6 and/or 5E1E4); and/or ECD3 binding properties that are similar or substantially equivalent to the binding properties of an appropriate ECD3-binding molecule as described herein (such as any one or more of 1D3, 1C10, 1A10, 1G4, 1G9 and/or 1E8).

In those contexts, a binding molecule according to the first aspect of the present invention can be considered to possess “similar or substantially equivalent to the binding properties” to 4H3C3, 7B1F3, 2F5B11, 4A11A11, 2G2B3, 5D6H11, 9G3B9, 5E8F7, 6G3D6 and/or 5E1E4 when tested under the same conditions as 4H3C3, 7B1F3, 2F5B11, 4A11A11, 2G2B3, 5D6H11, 9G3B9, 5E8F7, 6G3D6 and/or 5E1E4, respectively (and/or optionally 1D3, 1C10, 1A10, 1G4, 1G9 and/or 1E8, when tested under the same conditions as 1D3, 1C10, 1A10, 1G4, 1G9 and/or 1E8, respectively), if it displays similar or substantially equivalent binding specificity, similar or substantially equivalent strain agnostic binding properties, and/or similar or substantially equivalent binding affinity, to 4H3C3, 7B1F3, 2F5B11, 4A11A11, 2G2B3, 5D6H11, 9G33B9, 5E8F7, 6G3D6 and/or 5E1E4 (and/or optionally 1D3, 1C10, 1A10, 1G4, IG9 and/or 1E8). Such tests may, for example, correspond to any one or more of the tests reported in the present application for assessing the binding properties of 4H3C3, 7B1F3, 2F5B11, 4A11A11, 2G2B3, 5D6H11, 9G3B9, 5E8F7, 6G3D6 and/or 5E1E4 (and/or optionally 1D3, 1C10, 1A10, 1G4, 1G9 and/or 1E8), for example, any one or more of the tests conducted to determine binding to a polypeptide consisting of the amino acid sequence of TDVLNQSKPVTL (SEQ ID NO: 177), to US28 ECD3 peptides, on US28 overexpressing US28-CHO-A1 cells, HCMV Ad169 infected MRC-5 cells, primary PBMCs from HCMV seropositive individuals, HCMV infected human lung tissue and several types of aggressive human tumors, such as oesophagus, gastric, rectum, liver, lung, pancreas, cervical cancers, malignant pheochromocytoma and locally advanced colon cancer, breast cancer and its metastasis and glioblastoma grade 4.

A binding molecule according to the first aspect of the present invention may be considered to have similar or substantially equivalent binding specificity to one or more ECD1- and/or ECD3-reference binding molecule(s) of the present invention if, under any one or more tests to determine the ability to bind specifically to US28 ECD1 and/or ECD3 peptides (compared to a negative control, such as BSA), US28-expressing cells (compared to equivalent cells not expressing US28), HCMV infected cells (compared to equivalent cells without HCMV infection), primary PBMCs from HCMV seropositive individuals (compared to primary PBMCs from HCMV seronegative individuals), HCMV infected human lung tissue (compared to human lung tissue that is not HCMV infected) and/or types of HCMV-infected human tumors, in particular aggressive tumors, such as oesophagus, gastric, rectum, liver, lung, pancreas, cervical cancers, malignant pheochromocytoma and locally advanced colon cancer, breast cancer and its metastasis and glioblastoma grade 4 (compared to equivalent non-cancerous cells that are not infected with HCMV), the binding molecule displays at least 1, 5, 10, 15, 20, 25, 30, 40, 50, 60, 70, 80, 90, 95, or substantially 100% of the binding specificity of the respective ECD1- and/or ECD3-reference binding molecule(s). Certain binding molecules according the first aspect of the present invention may have a similar or substantially equivalent binding specificity to the binding specificity of the ECD1- and/or ECD3-reference binding molecule(s) that is nevertheless lower than the binding specificity of the respective ECD1- and/or ECD3-reference binding molecule, under any one or more of the foregoing tests; whereas certain other binding molecules according the first aspect of the present invention may have a higher binding specificity than the respective ECD1- and/or ECD3-reference binding molecule under any one or more of the foregoing tests.

A binding molecule according to the first aspect of the present invention may be considered to have similar or substantially equivalent strain agnostic binding properties to one or more ECD1- and/or ECD3-reference binding molecule(s) if, under any one or more tests to determine the ability to bind specifically to the ECD1 conserved epitope of SEQ ID NO: 177 as described herein and/or each of 4D- and 4N-variant US28 ECD3 peptides (all compared to a negative control, such as BSA), to each of 4D- and 4N-variant US28-expressing cells (each compared to equivalent cells not expressing US28; and optionally wherein the 4D- and 4N-variants of US28 are, respectively, the US28 sequences encoded by strains TB40/E and DB of HCMV, and/or further optionally wherein the cells are CHO cells), each of 4D- and 4N-variant US28-encoding HCMV strain infected cells (each compared to equivalent cells without HCMV infection), to primary PBMCs from each of 4D- and 4N-variant US28-encoding HCMV strain seropositive individuals (compared to primary PBMCs from HCMV seronegative individuals), to each of 4D- and 4N-variant US28-encoding HCMV strain infected human lung tissue (compared to human lung tissue that is not HCMV infected) and/or to each of 4D- and 4N-variant US28-encoding HCMV strain types of HCMV-infected human tumors, in particular aggressive tumors, such as oesophagus, gastric, rectum, liver, lung, pancreas, cervical cancers, malignant pheochromocytoma and locally advanced colon cancer, breast cancer and its metastasis and glioblastoma grade 4 (compared to equivalent non-cancerous cells that are not infected with HCMV), the binding molecule displays binding irrespective of the HCMV strain in comparison to the ECD1-reference binding molecule and/or an equality of binding to the 4D- and 4N-variant that is similar or substantially identical (e.g. ±30, 25, 20, 15, 10, 9, 8, 7, 6, 5, 4, 3, 2, 1% or less) to the equality of binding shown by the ECD3-reference binding molecule.

A binding molecule according to the first aspect of the present invention may be considered to have similar or substantially equivalent background binding to one or more ECD1- and/or ECD3-reference binding molecule(s) if, under any one or more tests to determine the background binding to cells that do not express US28 (compared to equivalent cells engineered to express US28), HCMV non-infected cells (compared to equivalent cells with HCMV infection), primary PBMCs from HCMV seronegative individuals (compared to primary PBMCs from HCMV seropositive individuals), HCMV non-infected human lung tissue (compared to human lung tissue that is HCMV infected) and/or types of HCMV non-infected human tumors (compared to equivalent cancerous cells that are infected with HCMV, in particular aggressive tumors, such as oesophagus, gastric, rectum, liver, lung, pancreas, cervical cancers, malignant pheochromnocytoma and locally advanced colon cancer, breast cancer and its metastasis and glioblastoma grade 4), the background binding molecule displays at least 1, 5, 10, 15, 20, 25, 30, 40, 50, 60, 70, 80, 90, 95, or substantially 100% of the background binding of the respective ECD1- and/or ECD3-reference binding molecule. Certain binding molecules according the first aspect of the present invention may have a similar or substantially equivalent background binding to the background binding of one or more ECD1- and/or ECD3-reference binding molecule(s) that is nevertheless higher than the background binding of the respective ECD1- and/or ECD3-reference binding molecule, under any one or more of the foregoing tests; whereas certain other binding molecules according the first aspect of the present invention may have lower binding specificity than the respective ECD1- and/or ECD3-reference binding molecule under any one or more of the foregoing tests. Alternatively, or additionally, certain binding molecules according the first aspect of the present invention may have lower background binding (e.g. less than 95, 90, 80, 70, 60, 50, 40, 30, 25, 20, 15, 10, 5, 1%, or less than 1%) compared to the background binding of VUN100 (monovalent VUN100 and/or bivalent VUN100), under any one or more of the foregoing tests.

A binding molecule according to the first aspect of the present invention may be considered to have (e.g. ±20, 15, 10, 9, 8, 7, 6, 5, 4, 3, 2, 1% or less) substantially equivalent binding affinity to one or more ECD1- and/or ECD3-reference binding molecule(s) if, under any one or more tests to determine the binding affinity to US28 ECD1 and/or ECD3 peptides, US28-expressing cells, HCMV infected cells, primary PBMCs from HCMV seropositive individuals, HCMV infected human lung tissue and/or types of HCMV-infected human tumors, in particular aggressive tumors, such as oesophagus, gastric, rectum, liver, lung, pancreas, cervical cancers, malignant pheochromocytoma and locally advanced colon cancer, breast cancer and its metastasis and glioblastoma grade 4, the binding molecule displays a binding affinity that is within, for example, 6, 5, 4, 3, 2 or 1 orders of magnitude of the binding affinity of the respective ECD1- and/or ECD3-reference binding molecule, when expressed as Kd. Certain binding molecules according the first aspect of the present invention may have a lower binding affinity than one or more ECD1- and/or ECD3-reference binding molecule(s) under any one or more of the foregoing tests; whereas certain other binding molecules according the first aspect of the present invention may have a higher binding affinity than one or more ECD1- and/or ECD3-reference binding molecule(s) under any one or more of the foregoing tests. It is noted that higher binding affinity is not necessarily always desirable, for example in the context of CARs wherein particularly high binding affinity can be less preferable, and CARs with higher binding specificity generally lead to greater therapeutic benefit than CARs with high binding affinity.

A binding molecule according the first aspect of the present invention may be considered to have any one or more of the aforementioned properties. For example, a binding molecule may be considered to have one or more of the following properties (each of which is described in more detail above):
1. similar or substantially equivalent binding specificity to one or more ECD1-reference binding molecule;
2. similar or substantially equivalent binding specificity to one or more ECD3-reference binding molecule;
3. similar or substantially equivalent strain agnostic binding properties to one or more ECD1-reference binding molecule;
4. similar or substantially equivalent strain agnostic binding properties to one or more ECD3-reference binding molecule;
5. similar or substantially equivalent background binding to one or more ECD1-reference binding molecule;
6. similar or substantially equivalent background binding to one or more ECD3-reference binding molecule;
7. lower background binding than the background binding of VUN100 (monovalent VUN100 and/or bivalent VUN100);
8. greater strain agnostic binding than the binding of VUN100 (monovalent VUN100 and/or bivalent VUN100);
9. higher binding specificity than the binding of VUN100 (monovalent VUN100 and/or bivalent VUN100);
10. substantially equivalent binding affinity to one or more ECD1-reference binding molecule; and/or
11. substantially equivalent binding affinity to one or more ECD3-reference binding molecule.

For example, a binding molecule may have (i) similar or substantially equivalent strain agnostic binding properties to one or more ECD1- and/or ECD3-reference binding molecule; and (ii) similar or substantially equivalent background binding to one or more ECD1- and/or ECD3-reference binding molecule. In other embodiments, a binding molecule may have (i) similar or substantially equivalent strain agnostic binding properties to one or more ECD1- and/or ECD3-reference binding molecule; and (ii) lower background binding to the background binding of VUN100 (monovalent VUN100 and/or bivalent VUN100). In other embodiments, a binding molecule may have (i) similar or substantially equivalent strain agnostic binding properties to one or more ECD1- and/or ECD3-reference binding molecule; (ii) similar or substantially equivalent background binding to one or more ECD1- and/or ECD3-reference binding molecule; and (iii) lower background binding to the background binding of VUN100 (monovalent VUN100 and/or bivalent VUN100). In other embodiments, a binding molecule may have (i) similar or substantially equivalent strain agnostic binding properties to one or more ECD1- and/or ECD3-reference binding molecule; (ii) similar or substantially equivalent background binding to one or more ECD1- and/or ECD3-reference binding molecule; (iii) lower background binding to the background binding of VUN100 (monovalent VUN100 and/or bivalent VUN100); and (iv) similar or substantially equivalent binding specificity to one or more ECD1- and/or ECD3-reference binding molecule.

Further optional assays for charactering the binding properties of binding molecule according to the first aspect of the present invention are discussed below.

### (a) Assay 1:

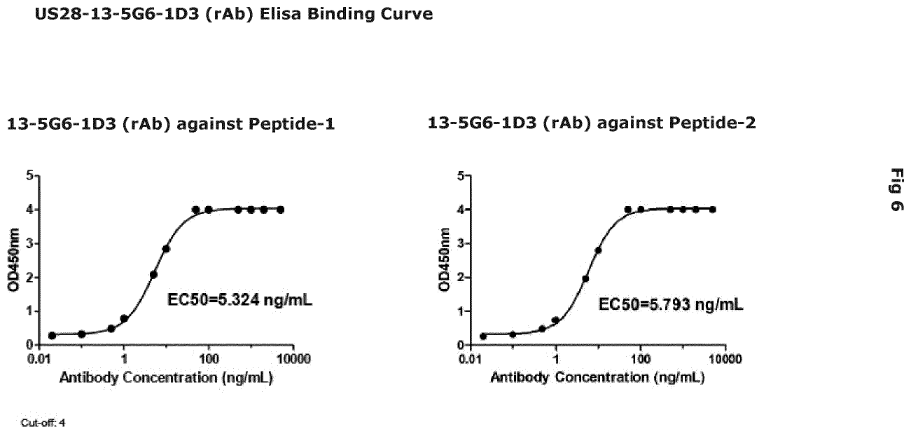

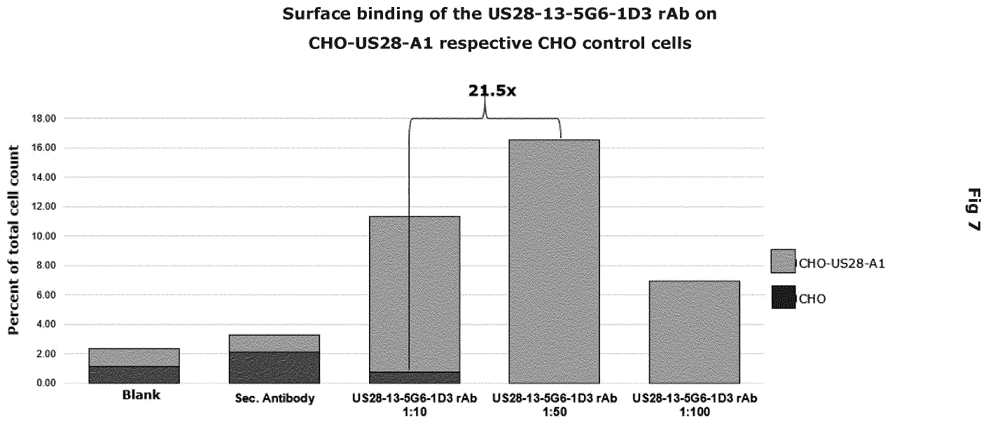

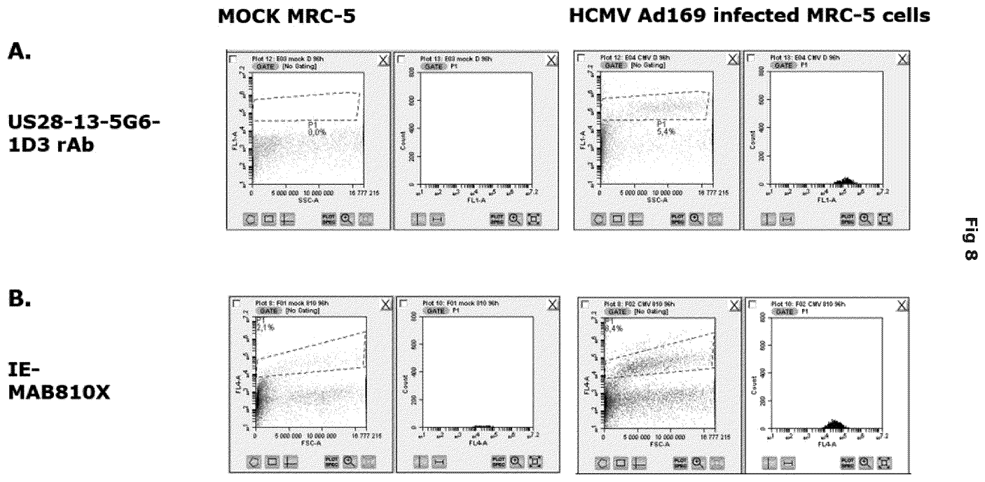

As shown in FIG. 5 of the present application, an exemplary ECD3 binding molecule according to the present invention, monoclonal antibody 1D3 that was raised against peptide sequence derived from ECD3 of US28, possesses the ability to bind specifically to peptide sequences derived from ECD3 of US28, compared to a negative control BSA.

The peptide sequences used in that assay were designated Peptide-1, having the sequence TKKNNQCMTDYDYLEVS (SEQ ID NO: 6, corresponding to the 4N variant of ECD3) and Peptide-2, having the sequence of TKKDNQCMTDYDYLEVS (SEQ ID NO: 7, corresponding to the 4D variant of ECD3).

As indicated in FIG. 5, the 1D3 antibody shows approximately 27 to 28-fold more specific binding to each of Peptide 1 and 2, compared to BSA control; and the binding levels that are observed of 1D3 to Peptides 1 and 2, both as an absolute level and when normalised against the respective BSA controls, are essentially identical (e.g. less than 5% difference, and likely within the region of experimental error). These results clearly show that 1D3 is both specific for ECD3 of US28, and also strain agnostic.

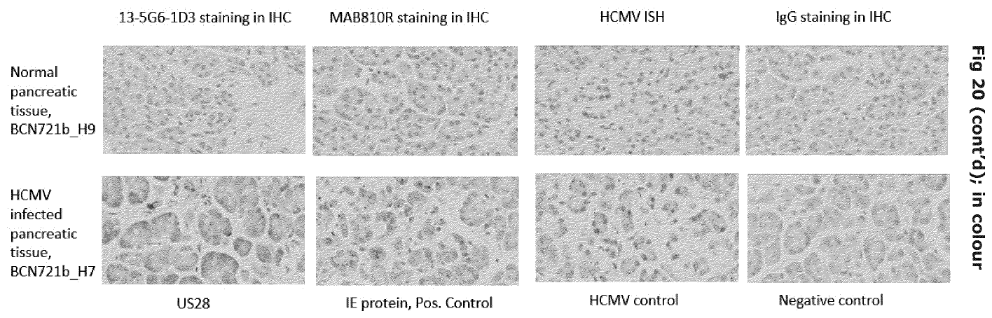

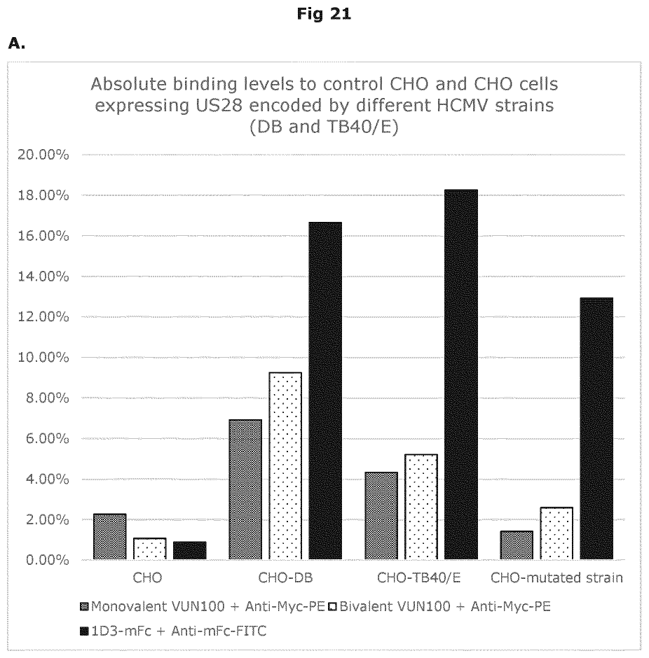

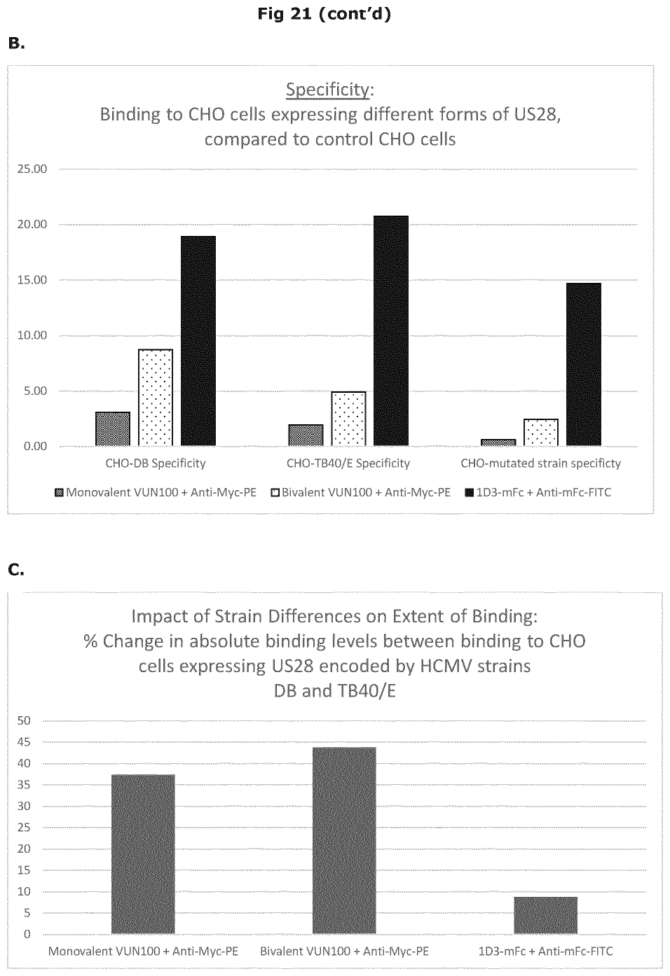

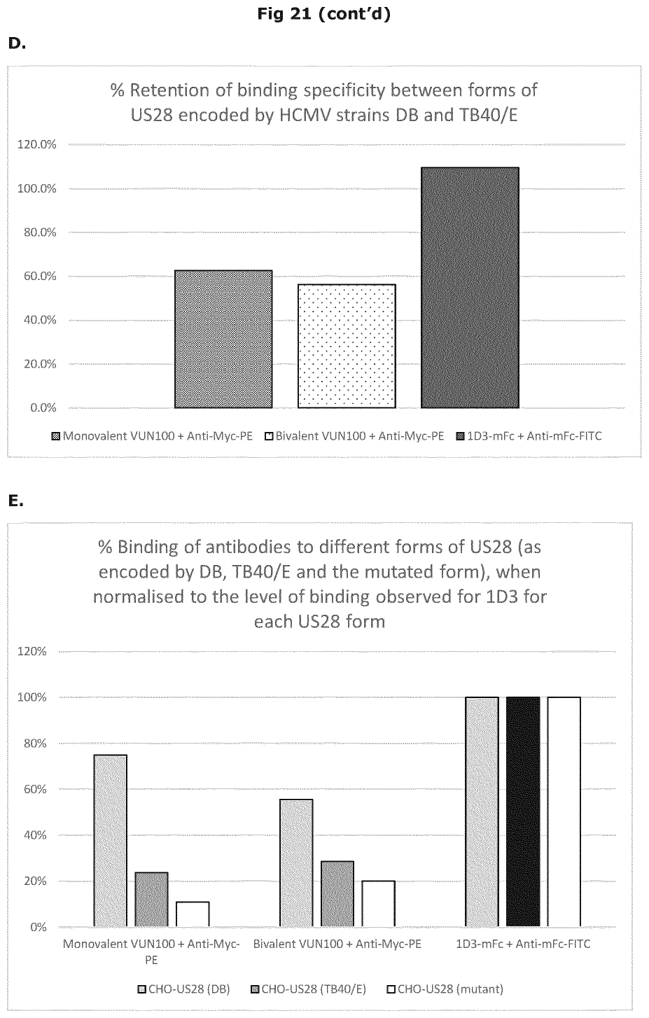

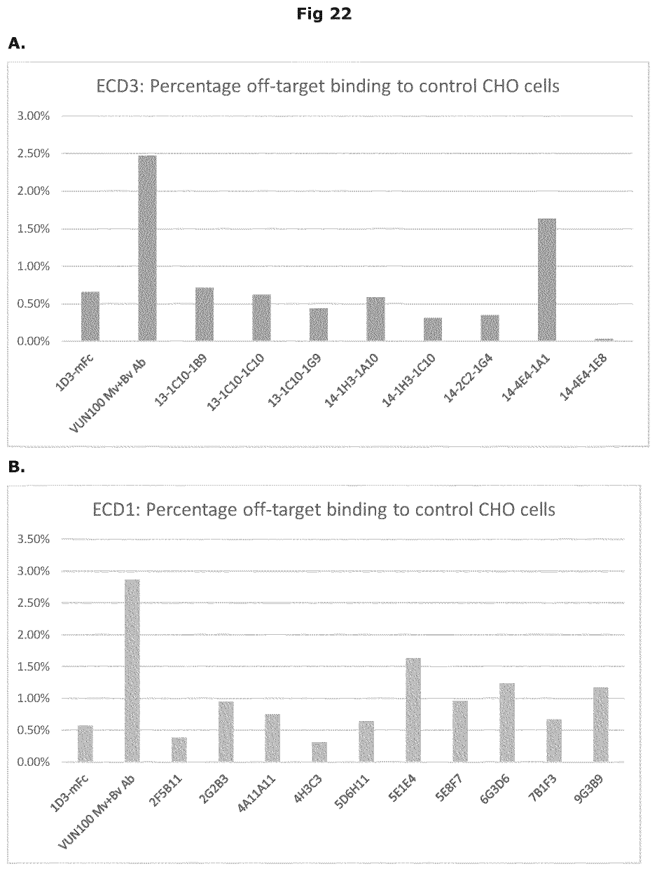

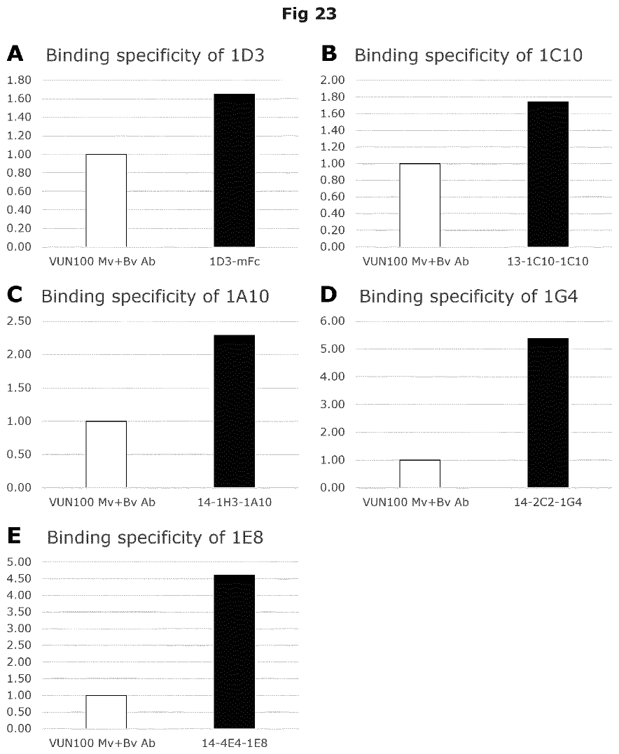

In contrast, the binding properties of VUN1.0 of WO 2019/151865 (having the sequence of SEQ ID NO: 60 of the present application) are very different. It is reported to bind to an epitope in the N-terminal region of US28, a region of US28 that possesses high levels of sequence variation between the different HCMV strains. VUN100 is shown to have a relatively low level of binding specificity; a specificity score of 4 was reported by De Groof et al., 2019 (supra) in their supporting information, FIG. S1.A thereof, and Table 2 of the present application. In addition, VUN100 shows a considerable difference in binding between the VHL/E, Merlin and TB40/E strains of HCMV, with binding being particularly reduced in strain TB40/E (B1 type) at around only half the level of binding observed against the Merlin strain (FIG. 8D of WO 2019/151865). Further characterisation of VUN100 is reported in a pre-printed article available online by De Groof et al, 2020 (doi: https://doi.org/10.1101/2020.05.12.071860), wherein FIG. 2 of the supplementary data gives the results of the % of induced HCMV IE-positive CD14+ monocytes bound by VUN100 from four different HCMV seropositive donors, wherein the strain(s) of HCMV within each donor is undetermined. The level of IE positivity induced by VUN100 binding to these cells varied by up to 14-fold between the different donors. This may be seen as a further indication that the binding ability of VUN100 varies considerably between cells infected with different strains of HCMV. These disadvantageous properties of VUN100, in comparison to the binding molecules of the present invention, are further demonstrated in the Examples of the present application, with particular reference to FIG. 21A-E, FIG. 22 and FIG. 23.

Accordingly, in one embodiment, a binding molecule of the first aspect of the present invention having anti-ECD3 binding specificity in addition to the anti-SEQ ID NO: 177 binding specificity (and/or a second binding molecule having anti-ECD3 binding specificity with which a binding molecule of the first aspect of the present invention may be combined in a formulation) may be characterised as having binding specificity to an epitope within ECD3 of a US28 protein of HCMV if it displays greater binding to Peptide 1 and/or Peptide 2 (preferably both), compared to a negative control (such as BSA), in a binding assay under conditions in which a reference antibody, wherein the reference antibody is 1D3, displays greater binding to Peptide 1 and/or Peptide 2 (preferably both), compared to the same negative control. In another embodiment, the reference binding molecule may be selected from the group consisting of 1C10, 1A10, 1G4, 1G9 and/or 1E8.

In an additional or alternative embodiment, a binding molecule of the first aspect of the present invention having anti-SEQ ID NO: 177 binding specificity (optionally wherein said binding molecule additionally possesses the anti-ECD3 binding specificity, and/or a first binding molecule having anti-SEQ ID NO: 177 binding specificity with which a binding molecule having anti-ECD3 binding specificity may be combined in a formulation) may be characterised as having binding specificity to an epitope within SEQ ID NO: 177 of a US28 protein of HCMV if it displays greater binding to Peptide-N, compared to a negative control (such as BSA), in a binding assay under conditions in which a reference antibody, wherein the reference antibody is 4H3C3, 7B1F3 and/or 2F5B11, displays greater binding to Peptide-N, compared to the same negative control.

In an additional or alternative embodiment, a binding molecule of the first aspect of the present invention having anti-ECD3 binding specificity in addition to the anti-SEQ ID NO: 177 specificity (and/or a second binding molecule having anti-ECD3 binding specificity with which a binding molecule of the first aspect of the present invention may be combined in a formulation) may be characterised as having strain agnostic binding to ECD3 if the respective binding levels that are observed of the binding molecule to each of Peptides 1 and 2 in the aforementioned assay, either as an absolute level and/or when normalised against the binding level observed to the respective negative control (such as BSA) control, are essentially identical, such as within 30%, 25%, 20%, 15%, 10%, 5%, 4%, 3%, 2%, 1% or less of each other.

The binding specificity of a binding molecule of the first aspect of the present invention having anti-ECD3 binding specificity in addition to the anti-SEQ ID NO: 177 specificity (and/or a second binding molecule having anti-ECD3 binding specificity with which a binding molecule of the first aspect of the present invention may be combined in a formulation) to an epitope within ECD3 of a US28 protein may, for example, be assessed in an ELISA, such as a methodology as described in the examples of this application, or by a variant of said methodology, such as by coating peptides derived from ECD3 (such as Bio-Peptide 1 and/or Bio-Peptide-2) on a first set of wells in a microtiter plate and coating a negative control (such as BSA) in a second set of wells in a microtiter plate. The first set of wells may optionally be further subdivided into a first subgroup of the first set of wells that comprises the sequence of a 4N-variant from ECD3 of US28, and a second subgroup of the first set of wells that comprises the sequence of a 4D-variant from ECD3 of US28, to permit the determination of the relative binding specificity to each variant. Following the coating of the first and second sets of wells, a binding molecule of interest may be incubated in each of the first and second sets of wells, optionally with additional testing of a positive control (e.g. any one or more of 1D3, 1C10, 1A10, 1G4, 1G9 and/or 1E8) in respective first and second sets of wells. Following incubation, the wells may be washed and then incubated with an enzyme-conjugated secondary antibody. Following a further wash, a substrate for the conjugated enzyme can be added that undergoes a measurable reaction (e.g. a colour change, the absorbance of) which correlates with the amount of binding for the binding molecule and controls, which gives an indication of binding specificity and/or strain agnostic binding characteristics. Accordingly, in some embodiments, the binding molecule of interest has an absorbance value following ELISA that is indicative of positive binding to one or more (preferably both) of Peptides 1 and/or 2 derived from ECD3 in the first set of wells, compared to the binding to the negative control (such as BSA).

By “positive binding”, we include that the binding molecule of interest can have a higher level of binding to one or more (preferably both) of Peptides 1 and/or 2 compared with the level of binding to a negative control (e.g. BSA), which indicates a higher binding specificity.

It may, for example, display at least about 2-fold, at least about 5-fold, at least about 10-fold, at least 15-fold, at least 20-fold, at least 25-fold, about 26-fold, about 27-fold, or about 28-fold greater binding to Peptide 1 and/or Peptide 2, compared to a negative control such as BSA under conditions in which antibody 1D3 displays about 27 to 28-fold greater binding to Peptide 1 and/or Peptide 2, compared to a negative control such as BSA. The term “about” as used in this context can include values that are ±50%, 40%, 30%, 20%, 15%, 10%, 9%, 8%, 7%, 6%, 5%, 4%, 3%, 2% or 1% of the stated value (for example, ±10% of at least about 10-fold refers to the range of from 9-fold to 11-fold). For example, the conditions for such an assay may be selected to be the same as, or equivalent to, the conditions used in the assay used in generating the results shown in FIG. 5.

Alternatively, or additionally, “positive binding” can include that the binding molecule of interest has a similar (e.g. at least ±50%, 40%, 30%, 20%, 10%, 5% or less), or higher/increased/enhanced/improved, absorbance level compared with a positive control (e.g. 1D3, 1C10, 1A10, 1G4, 1G9 and/or IE8).

### (b) Assay 2:

In a further embodiment, it is preferred that a binding molecule of the present invention binds preferentially to US28-expressing cells (such as CHO cells engineered to express US28) compared to its binding of US28-negative cells (such as wildtype CHO cells), for example, at a 1:50 (w/v) dilution, for example as determined by flow cytometry assay. US28-expressing cells are preferably cells that are characterised by displaying cell surface expression of the US28 protein. Surface expression of US28 is typically characterised by the display of the extracellular domains of US28 (ECDs 1, 2, 3 and 4 corresponding, respectively, to positions 1-37, positions 91-101, positions 167-183 and positions 250-273 of the US28 protein encoded by HCMV strain DE as defined by the sequence of SEQ ID NO: 5, or equivalent sequences of other HCMV strains) on the cell surface.

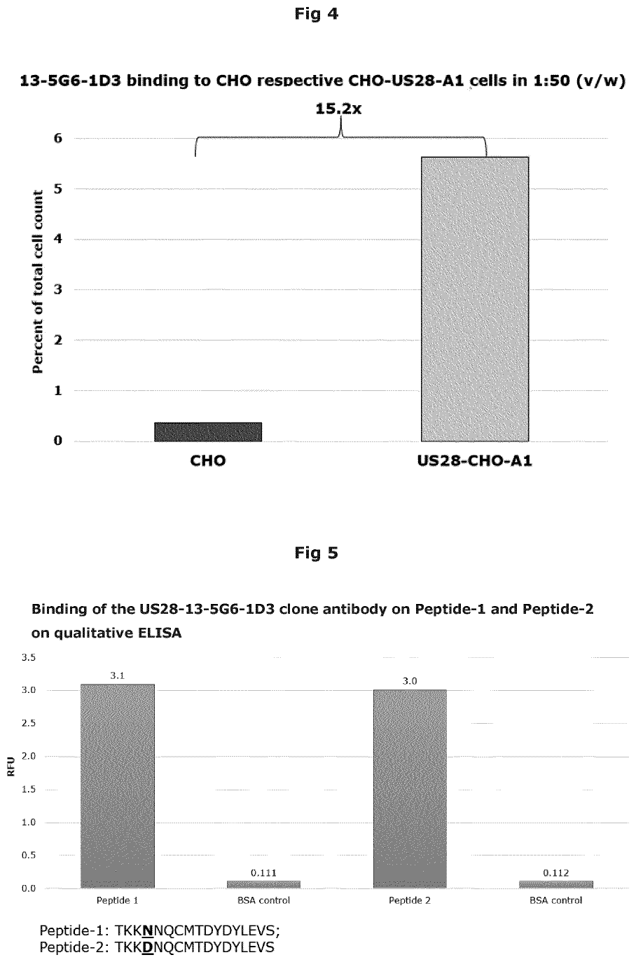

FIGS. 4, 7 and Table 2 of the present application demonstrates that an exemplary anti-ECD3 binding molecule of the present application, monoclonal antibody 1D3 raised against ECD3 of US28, possess the ability to bind specifically to US28-expressing Chinese Hamster Ovary (CHO-US28-A1) cells compared to US28-negative control CHO cells with a specificity score of between around 15 to 21.5 under the binding conditions used. This can be assayed using any suitable technique, for example using FACS analysis, to determine the percentage of total cell count bound. As further reported in Example 1 of the present application, with particular reference to Table 2, although clone US28-13-5G6-1D3 (encoding antibody 1D3) was most fully characterised, other cloned monoclonal antibodies raised against ECD3 of US28 showed excellent binding specificity to CHO-US28-A1 cells, when compared with their binding to US28-negative control CHO cells. As further reported in Example 2, yet further other cloned monoclonal antibodies raised against ECD3 of US28 showed excellent binding specificity to CHO-US28-A1 cells, when compared with their binding to US28-negative control CHO cells, in particular having very low off-target binding (e.g. compared to the much higher levels of off-target binding demonstrated by VUN100) and in numerous instances also showing higher binding specificity to CHO-US28-A1 cells, when compared with their binding to US28-negative control CHO cells.

These data clearly demonstrate that, by following the teachings of the present application, there is provided a consistent route to the generation of antibodies raised against ECD3 of US28 that can provide highly specific binding to US28-expressing cells (such as CHO cells) compared to the binding of US28-negative cells (such as US28-negative CHO cells), preferably with low off-target binding levels, a set of characteristics not shared by VUN100.

In contrast, the binding properties of VUN100 of WO 2019/151865 (having the sequence of SEQ ID NO: 60 of the present application) are very different. It is reported to bind to a discontinuous epitope between the N-terminal and ECD4 regions of US28, within a region of US28 that possesses high levels of sequence variation between the different HCMV strains. VUN100 is shown to have a relatively low level of binding specificity: a specificity score of 4 was reported by De Groof et al., 2019 (supra) in their supporting information, FIG. S1.A thereof (and summarised in Table 2 of the present application) when tested for binding to either US28-expressing HEK293T membranes (HEK+US28) or mock transfected HEK293T membranes. VUN100 is clearly not capable of providing highly specific binding to US28. These disadvantageous properties of VUN100, in comparison to the binding molecules of the present invention, are further demonstrated in the Examples of the present application, with particular reference to FIG. 21A-E, FIG. 22, FIG. 23 and FIG. 24.

Accordingly, in one preferred embodiment, a binding molecule of the first aspect of the present invention will display specific binding, characterised in that its binding specificity for US28-expressing cells (such as CHO-US28-A1 cells, optionally wherein the US28 sequence corresponds to the sequence encoded by strain DB or TB40/E of HCMV) compared to US28-negative cells (such as CHO cells) is greater than the level of specificity achieved by VUN100. For example, the level of specificity a binding molecule of the first aspect of the present invention may be greater than the level of specificity achieved by VUN100 in the same assay by at least about 10%, about 20%, about 30%, about 40%, about 50%, about 60%, about 80%, about 90%, about 100% (i.e. about 2-fold), about 150%, about 200% (i.e. about 3-fold), about 300% (i.e. about 4-fold), about 400% (i.e. about 5-fold) or about 500% (i.e. about 6-fold), at the same molar concentration. In this context, the VUN100 comparator may refer to a polypeptide comprising, consisting essentially of, or consisting of, the sequence of any one or more of the sequences of SEQ ID NOs: 60-64 (for example, monovalent VUN100, bivalent VUN100, or a mix thereof such as a 1:1 mix thereof, each as described further herein). In the context of SEQ ID NOs: 61 and 63, this may include proteins with, or without, the indicated signal peptide sequences.

In another preferred embodiment, a binding molecule of the first aspect of the present invention will display specific binding, characterised in that its binding specificity for US28-expressing cells (such as CHO-US28-A1 cells, optionally wherein the US28 sequence corresponds to the sequence encoded by strain DB or TB40/E of HCMV) compared to US28-negative cells (such as CHO cells) is at least about the same level of specificity as any one or more of 4H3C3, 7B1F3, 2F5B11, 4A11A11, 2G2B3, 5D6H11, 9G3B9, 5E8F7, 6G3D6 and/or 5E1E4 (and/or optionally 1D3, 1C10, 1A10, 1G4 and/or 1E8) in the same assay, at the same molar concentration. The term “about” as used in this context can include values that are ±80%, 70%, 60%, 50%, 40%, 30%, 20%, 15%, 10%, 9%, 8%, 7%, 6%, 5%, 4%, 3%, 2% or 1% of the level of specificity of any one or more of 1D3, 1C10, 1A10, 1G4 and/or 1E8 in the same assay, at the same molar concentration.

In this context, the 4H3C3, 7B1F3 and/or 2F5B11 comparator(s) may refer to an antibody molecule comprising, consisting essentially of, or consisting of:
the heavy chain polypeptide sequence of 4H3C3 as defined by SEQ ID NO: 180 (after removal of the indicated N-terminal leader sequence) and light chain polypeptide sequence of 4H3C3 as defined by SEQ ID NO: 181 (after removal of the indicated N-terminal leader sequence);
the heavy chain polypeptide sequence of 7B1F3 as defined by SEQ ID NO: 204 (after removal of the indicated N-terminal leader sequence) and light chain polypeptide sequence of 7B1F3 as defined by SEQ ID NO: 205 (after removal of the indicated N-terminal leader sequence); or
the heavy chain polypeptide sequence of 2F5B11 as defined by SEQ ID NO: 226 (after removal of the indicated N-terminal leader sequence) and light chain polypeptide sequence of 2F5311 as defined by SEQ ID NO: 227 (after removal of the indicated N-terminal leader sequence).

Optionally, in this context, the 1D3, 1C10, 1A10 and/or 1E8 comparator(s) may refer to an antibody molecule comprising, consisting essentially of, or consisting of:
the heavy chain polypeptide sequence of 1D3 as defined by SEQ ID NO: 20 (after removal of the indicated N-terminal leader sequence) and light chain polypeptide sequence of 1D3 as defined by SEQ ID NO: 21 (after removal of the indicated N-terminal leader sequence);
the heavy chain polypeptide sequence of 1C10 as defined by SEQ ID NO: 157 (after removal of the indicated N-terminal leader sequence) and light chain polypeptide sequence of 1C10 as defined by SEQ ID NO: 158 (after removal of the indicated N-terminal leader sequence);
the heavy chain polypeptide sequence of 1A10 as defined by SEQ ID NO: 161 (after removal of the indicated N-terminal leader sequence) and light chain polypeptide sequence of 1A10 as defined by SEQ ID NO: 162 (after removal of the indicated N-terminal leader sequence); or
the heavy chain polypeptide sequence of 1E8 as defined by SEQ ID NO: 153 (after removal of the indicated N-terminal leader sequence) and light chain polypeptide sequence of 1E8 as defined by SEQ ID NO: 154 (after removal of the indicated N-terminal leader sequence).

Such assays can, for example, be performed to determine the relative binding to US28-expressing cells (such as CHO-US28-A1 cells, optionally wherein the US28 sequence corresponds to the sequence encoded by strain DB or TB40/E of HCMV) and to US28-negative cells (such as CHO cells), for example, at a molar ratio equivalent to a 1:5 (w/v) dilution of 4H3C3, 7B1F3 and/or 2F5B11; and optionally a 1:50 (w/v) dilution of 1D3, 1C10, 1A10 and/or 1E8; for example as determined by flow cytometry assay.

### (c) Assay 3:

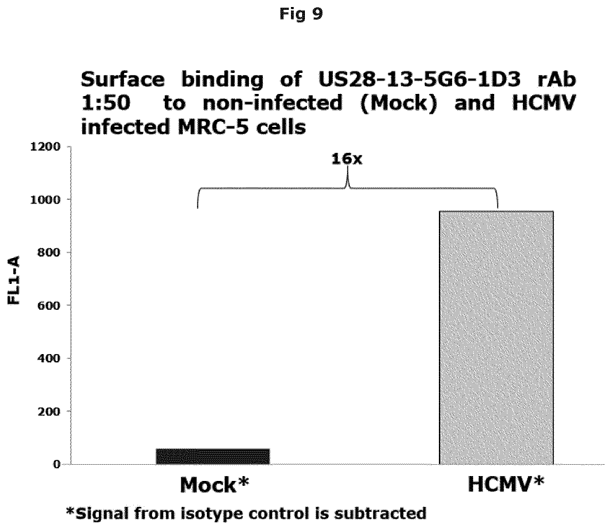

In a further embodiment, it is preferred that a binding molecule of the first aspect of the present invention can bind preferentially to HCMV-infected cells (such as MRC-5 cells; cat #CCL-171, RRID: CVCL_0440, American Type Culture Collection (ATCC), Manassas, VA 20110 USA) compared to its binding of equivalent cells without HCMV infection. As shown in FIG. 8 of the present application, an exemplary ECD3-binding molecule according to the present application, monoclonal antibody 1D3 that was raised against ECD3 of US28, possess the ability to bind to HCMV-infected MRC-5 cells but not to the non-infected (mock) cells, when assayed by using flowcytometry analysis. Under the assay conditions used, 1D3 showed about 5.4% binding to HCMV-infected cells, compared to 0% binding to the non-infected (mock) cells. Further testing, as shown in FIG. 9, showed that 1D3 binds specifically (with approximately 16-fold greater specificity) to the surface of the HCMV infected MRC-5 cells as compared with the uninfected cells.

Accordingly, in a further preferred embodiment, a binding molecule of the first aspect of the present invention will display preferential binding to HCMV-infected cells (such as MRC-5 cells) that is, or is at least, 5-fold, about 6-fold, about 7-fold, about 8-fold, about 9-fold, about 10-fold, about 11-fold, about 12-fold, about 13-fold, about 14-fold, about 15-fold or about 16-fold greater, compared to its binding of equivalent cells without HCMV infection. Such an assay may, for example, be conducted in accordance with the protocol used to generate the results shown in FIG. 8 or 9 of the present application, or with an equivalent protocol. The term “about” as used in this context can include values that are ±50%, 40%, 30%, 20%, 15%, 10%, 9%, 8%, 7%, 6%, 5%, 4%, 3%, 2% or 1% of the stated value.

In a further preferred embodiment, a binding molecule of the first aspect of the present invention will display preferential binding to HCMV-infected cells (such as MRC-5 cells) compared to its binding of equivalent cells without HCMV infection that is greater than the level of preferential binding achieved by VUN100. For example, the level of preferential binding of a binding molecule of first aspect of the present invention may be greater than the level of preferential binding achieved by VUN100 in the same assay by at least about 10%, about 20%, about 30%, about 40%, about 50%, about 60%, about 80%, about 90%, about 100% (i.e. about 2-fold), about 150%, about 200% (i.e. about 3-fold), about 300% (i.e. about 4-fold), about 400% (i.e. about 5-fold) or about 500% (i.e. about 6-fold), at the same molar concentration. In this context, the VUN100 comparator may refer to a polypeptide comprising, consisting essentially of, or consisting of, the sequence of any one or more of the sequences of SEQ ID NOs: 60-64 (for example, monovalent VUN100, bivalent VUN100, or a mix thereof such as a 1:1 mix thereof, each as described further herein). In the context of SEQ ID NOs: 61 and 63, this may include proteins with, or without, the indicated signal peptide sequences.

A binding molecule of the first aspect of the present invention can, for example, display preferential binding to HCMV-infected cells compared to its binding of equivalent cells without HCMV infection that is, or is at least, about 20%, 30%, 40%, 50%, 60%, 70%, 80%, 90%, 95%, 96%, 97%, 98%, 99% or substantially 100% of the preferential binding activity of a reference antibody of the present invention (e.g. any one or more of 4H3C3, 7B1F3, 2F5B11, 4A11A11, 2G2B3, 5D6H11, 9G3B9, 5E8F7, 6G3D6 and/or 5E1E4, and optionally any one or more of 1D3, 1C10, 1A10, 1G4, 1G9 and/or 1E8) under the same conditions. The term “about” as used in this context can include values that are ±50%, 40%, 30%, 20%, 15%, 10%, 9%, 8%, 7%, 6%, 5%, 4%, 3%, 2% or 1% of the stated value (for example, +50% of 10% refers to the range of 5% to 15%). For example, the conditions for such an assay may be selected to be the same as, or equivalent to, the conditions used in the assay used in generating the results shown in FIG. 8 or 9.

### (d) Assay 4:

In a further embodiment, it is preferred that a binding molecule of the first aspect of the present invention can bind preferentially to an epitope within SEQ ID NO: 177, as compared with any one or more, such as all, of the markers denoted by “List A” and/or “List B” (see Example 4). A preferential binding to the epitope within SEQ ID NO: 177 can be assessed using, for example, “Peptide 3” (TDVLNQSKPVTL).

The determination of preferential binding optionally can be assessed using the techniques described in Example 4. For example, preferential binding may be at least 10-fold, 50-fold, 100-fold, 200-fold, 500-fold, 1,000-fold, 2,000-fold, 5,000-fold, 10,000-fold or more higher spot intensity (F635)/AU for Peptide 3 as compared with any one or more target from List A and/or List B, including but not limited to HSPD1 (also known as HSP60) and/or ITGA6 (integrin alpha 6, also known as CD49f).

Alternatively, or additionally, the determination of preferential binding can be assessed using the techniques described in Example 5. For example, preferential binding may be an increased hit rate (based on duplicate spots by analysing fluorescence (for example, using AF647 and ZsGreen 1) on ImageQuant) for Peptide 3 as compared with any one or more, such as all, of the other human plasma membrane proteins defined by List C and/or by the human heterodimers defined by List D.

### (e) Assay 5:

In a further embodiment, it is preferred that a binding molecule of the first aspect of the present invention demonstrate strain agnostic binding to an epitope within SEQ ID NO: 177 of US28. Strain agnostic binding can be assessed using, for example, US28 proteins encoded by two, three or all four of HCMV strains VHL/E, DB, TB40/E and/or Merlin.

The determination of strain agnostic binding can be assessed, for example, using the techniques described in Example 8. For example, strain agnostic binding may be characterised as showing a level of variation of binding between US28 proteins encoded by two, three or all four of HCMV strains VHL/E, DB, TB40/E and/or Merlin that is less than (such as at least 50, 40, 30, 20, 15, 14, 13, 12, 11, 10, 9, 8, 7, 6, 5, 4, 3, or 2 times less than) the level of variation of binding between the same US28 proteins as demonstrated by bivalent VUN100 as used in Example 8. Additionally, or alternatively, strain agnostic binding may be characterised as showing a level of variation of binding between US28 proteins encoded by two, three or all four of HCMV strains VHL/E, DB, TB40/E and/or Merlin that is substantially equivalent to the level of variation demonstrated by any one or more of antibodies 2F5B11, 4H3C3 and 7B1F3 as described in Example 8. In the context used herein, the term substantially equivalent may optionally include a level that is less than 50, 40, 30, 20, 15, 14, 13, 12, 11, 10, 9, 8, 7, 6, 5, 4, 3, or 2 different to the level of variation demonstrated by any one or more of antibodies 2F5B11, 4H3C3 and 7B1F3, when tested under identical conditions.

### Polypeptides

A binding molecule according to the first aspect of the present invention, comprises, consists essentially of, or consists of one or more polypeptide chains.

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. The term “amino acid” as used herein includes the standard twenty genetically-encoded amino acids and their corresponding stereoisomers in the ‘D’ form (as compared with the natural ‘L’ form), omega-amino acids other naturally-occurring amino acids, unconventional amino acids (e.g. α,α-disubstituted amino acids, N-alkyl amino acids, etc.) and chemically derivatised amino acids (see below).

Amino acids herein may be referred to by full name, three letter code or single letter code. When an amino acid is being specifically enumerated, such as “alanine” or “Ala” or “A”, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

In one embodiment, the polypeptides as defined herein comprise or consist of L-amino acids.

It will be appreciated by persons skilled in the art that the polypeptides of the present invention and/or as used as described herein in conjunction with the present invention, may comprise or consist of one or more amino acids which have been modified or derivatised.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine, and ornithine for lysine. Derivatives also include peptides containing one or more additions or deletions as long as the requisite activity is maintained. Other included modifications are amidation, amino terminal acylation (e.g. acetylation or thioglycolic acid amnidation), terminal carboxylamidation (e.g. with ammonia or methylamine), and the like terminal modifications.

It will be further appreciated by persons skilled in the art that peptidomimetic compounds may also be useful. The term ‘peptidomimetic’ refers to a compound that mimics the conformation and desirable features of a particular peptide as a therapeutic agent.

For example, the said polypeptide includes not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997, J. Immunol., 159(7): 3230-3237). This approach involves making pseudo-peptides containing changes involving the backbone, and not the orientation of side chains. Retro-inverse peptides, which contain NH—CO bonds instead of CO—NH peptide bonds, are much more resistant to proteolysis. Alternatively, the said polypeptide may be a peptidomimetic compound wherein one or more of the amino acid residues are linked by a -y(CH₂NH)— bond in place of the conventional amide linkage.

In a further alternative, the peptide bond may be dispensed with altogether provided that an appropriate linker moiety which retains the spacing between the carbon atoms of the amino acid residues is used; it may be advantageous for the linker moiety to have substantially the same charge distribution and substantially the same planarity as a peptide bond.

It will also be appreciated that the said polypeptide may conveniently be blocked at its N- or C-terminus so as to help reduce susceptibility to exo-proteolytic digestion.

A variety of un-coded or modified amino acids such as D-amino acids and N-methyl amino acids have also been used to modify mammalian peptides. In addition, a presumed bioactive conformation may be stabilised by a covalent modification, such as cyclisation or by incorporation of lactam or other types of bridges, for example see Veber et al. (1978, Proc. Natl. Acad. Sci. USA, 75:2636) and Thursell et al. (1983, Biochem. Biophys. Res. Comm. 111:166), which are incorporated herein by reference.

### Binding Molecule Structures:

A “binding molecule” in accordance with the first aspect of the present invention typically comprises, consists essentially of, or consists of, one or more polypeptides.

In some embodiments, a binding molecule (or a portion thereof) may be formed through the combination of multiple polypeptides. For example, a V_{H} polypeptide may be combined with a V_{L} polypeptide, therein forming a Fab fragment. The combination of polypeptides may be a V_{H} polypeptide from one exemplary ECD1-binding molecule combined with a V_{L} polypeptide of the same exemplary ECD1-binding molecule, or with a V_{L} polypeptide of a different exemplary ECD1-binding molecule. In multispecific formats further comprising an ECD3-binding domain, the combination of polypeptides for the ECD3-binding domain may be a V_{H} polypeptide from one exemplary ECD3-binding molecule combined with a V; polypeptide of the same exemplary ECD3-binding molecule, or with a V_{L} polypeptide of a different exemplary ECD3-binding molecule.

In some preferred embodiments, the binding molecule is selected from the group consisting of an antibody and a chimeric antigen receptor (CAR).

As discussed above, and further elucidated in Table 2 of Example 1, and also in Example 2, the present application describes numerous antibodies, raised against a polypeptide consisting of SEQ ID NO: 177 of ECD1, or ECD3, of US28 in accordance with the methods described herein, with highly beneficial binding properties to US28, including the ECD1 antibodies 4H3C3, 7B1F3, 2F5B11, 4A11A11, 2G2B3, 5D6H11, 9G3B9, 5E8F7, 6G3D6 and 5E1E4; and ECD3 antibodies 13-5G6-1D3 (generally abbreviated herein as “1D3”), 13-5C6-1B5, 14-1H3-1A6, 13-1C10-1C10 (generally abbreviated herein as “1C10”), 14-1H3-1A10 (generally abbreviated herein as “1A10”), 14-2C2-1G4 (generally abbreviated herein as “1G4”), 13-1C10-1G9 (generally abbreviated herein as “1G9”) and 14-4E4-1E8 (generally abbreviated herein as “1E8”).

The present application also provides a method of obtaining further antibodies having binding specificity to a first epitope within a polypeptide consisting of the amino acid sequence of TDVLNQSKPVTL (SEQ ID NO: 177) within the ECD1 of a US28 protein of HCMV, in accordance with the thirty-fourth aspect of the present invention, as discussed further in Section N of this application. For example, the method may comprise:
(a) providing one or more peptides corresponding an amino acid sequence present in a polypeptide consisting of the amino acid sequence of TDVLNQSKPVTL (SEQ ID NO: 177) within the ECD1 of the US28 protein, wherein the one or more peptides each comprise, consist essentially of, or consist of, the polypeptide sequence TDVLNQSKPVTL (SEQ ID NO: 177), and/or an immunogenic fragment thereof;
(b) providing one or more candidate antibodies (which have optionally been raised in response to said peptide); and
(c) determining the binding specificity and/or binding affinity of one or more candidate antibodies to said one or more peptide.

The present application also provides a method of obtaining further antibodies having binding specificity to an epitope within ECD3 of a US28 protein of HCMV, in accordance with the thirty-fourth aspect of the present invention, as discussed further in Section N of this application. For example, the method may comprise:
(a) providing one or more peptides corresponding an amino acid sequence present in ECD3 of the US28 protein, such as one or both that peptides comprise, consist essentially of, or consist of, the polypeptide sequence TKKDNQCMTDYDYLEVS (SEQ ID NO: 7) and/or TKKNNQCMTDYDYLEVS (SEQ ID NO: 6), and/or an immunogenic fragment of either or both;
(b) providing one or more candidate antibodies (which have optionally been raised in response to one or both of said peptides); and
(c) determining the binding specificity and/or binding affinity of one or more candidate antibodies to the one or both of said peptides.

In some preferred embodiments, the binding molecule of the first aspect of the present invention comprises one, two, three, four, five or six complementarity determining regions (CDRs) having binding specificity to the first epitope within a polypeptide consisting of the amino acid sequence of SEQ ID NO: 177 corresponding to any one, two, three, four, five or all six of the CDR sequences of antibody 4H3C3 as defined by SEQ ID NOs: 196, 197, 198, 199, 200 and 201, respectively, and/or a functional variant (such as a 4H3C3 humanised variant) of any one or more of said CDR sequences of antibody 4H3C3, and optionally wherein the binding molecule is selected from an antibody and a CAR.

The sequences, and identities of the CDR sequences of antibody 4H3C3 as defined by SEQ ID NOs: 196, 197, 198, 199, 200 and 201 are as follows:

| | | |
|---|---|---|
| SEQ ID | SYWMH | V_{H}-CDR1 sequence |
| NO: 196 | | of 4H3C3 |
| SEQ ID | YINPSTGYAEYNQKFKD | V_{H}-CDR2 sequence |
| NO: 197 | | of 4H3C3 |
| SEQ ID | LRFGSSGFAY | V_{H}-CDR3 sequence |
| NO: 198 | | of 4H3C3 |
| SEQ ID | KSSQSLLYSSNQKNYLA | V_{L}-CDR1 sequence |
| NO: 199 | | of 4H3C3 |
| SEQ ID | WASTWES | V_{L}-CDR2 sequence |
| NO: 200 | | of 4H3C3 |
| SEQ ID | QQYYSFPLT | V_{L}-CDR3 sequence |
| NO: 201 | | of 4H3C3 |

The sequences, and identities of the CDR sequences of a 4H3C3 humanised antibody may be defined by the following SEQ ID NOs:

| | | | |
|---|---|---|---|
| V_{H}-CDR1 | SEQ ID | SYWMH | V_{H}-CDR1 sequence |
| | NO: 196 | | of 4H3C3 |
| V_{H}-CDR2 | SEQ ID | YINPSTGYAE | V_{H}-CDR2 sequence |
| | NO: 197 | YNQKFKD | of 4H3C3 |
| | SEQ ID | YINPSTGYAE | |
| | NO: 293 | YNQKFQG | |
| | SEQ ID | YINPSTGYAE | |
| | NO: 294 | YNQKLQG | |
| V_{H}-CDR3 | SEQ ID | LRFGSSGFAY | V_{H}-CDR3 sequence |
| | NO: 198 | | of 4H3C3 |
| V_{L}-CDR1 | SEQ ID | KSSQSLLYSS | V_{L}-CDR1 sequence |
| | NO: 199 | NQKNYLA | of 4H3C3 |
| | SEQ ID | RSSQSLLYSS | |
| | NO: 295 | NQKNYLA | |
| V_{L}-CDR2 | SEQ ID | WASTWES | V_{L}-CDR2 sequence |
| | NO: 200 | | of 4H3C3 |
| | SEQ ID | WASTRES | |
| | NO: 222 | | |
| V_{L}-CDR3 | SEQ ID | QQYYSFPLT | V_{L}-CDR3 sequence |
| | NO: 201 | | of 4H3C3 |

In some preferred embodiments, the binding molecule of the first aspect of the present invention comprises one, two, three, four, five or six complementarity determining regions (CDRs) having binding specificity to the first epitope within a polypeptide consisting of the amino acid sequence of SEQ ID NO: 177 corresponding to any one, two, three, four, five or all six of the CDR sequences of antibody 7B1F3 as defined by SEQ ID NOs: 219, 220, 221, 199, 222 and 223, respectively, and/or a functional variant (such as a 7B1F3 humanised variant) of any one or more of said CDR sequences of antibody 7B1F3, and optionally wherein the binding molecule is selected from an antibody and a CAR.

The sequences, and identities of the CDR sequences of antibody 7B1F3 as defined by SEQ ID NOs: 219, 220, 221, 199, 222 and 223 are as follows:

| | | |
|---|---|---|
| SEQ ID | NYWMH | V_{H}-CDR1 sequence |
| NO: 219 | | of 7B1F3 |
| SEQ ID | YVNPRTGYTEYNQKFKD | V_{H}-CDR2 sequence |
| NO: 220 | | of 7B1F3 |
| SEQ ID | ILNGTGFAY | V_{H}-CDR3 sequence |
| NO: 221 | | of 7B1F3 |
| SEQ ID | KSSQSLLYSSNQKNYLA | V_{L}-CDR1 sequence |
| NO: 199 | | of 7B1F3 |
| SEQ ID | WASTRES | V_{L}-CDR2 sequence |
| NO: 222 | | of 7B1F3 |
| SEQ ID | QQYYSFPLT | V_{L}-CDR3 sequence |
| NO: 223 | | of 7B1F3 |

The sequences, and identities of the CDR sequences of a 7B1F3 humanised antibody may be defined by the following SEQ ID NOs:

| | | | |
|---|---|---|---|
| V_{H}-CDR1 | SEQ ID | NYWMH | V_{H}-CDR1 sequence |
| | NO: 219 | | of 7B1F3 |
| V_{H}-CDR2 | SEQ ID | YVNPRTGYT | V_{H}-CDR2 sequence |
| | NO: 220 | EYNQKFKD | of 7B1F3 |
| | SEQ ID | YVNPRTGYT | |
| | NO: 296 | EYNQKFQG | |
| | SEQ ID | YVNPRTGYT | |
| | NO: 297 | EYNQQLQG | |
| V_{H}-CDR3 | SEQ ID | ILNGTGFAY | V_{H}-CDR3 sequence |
| | NO: 221 | | of 7B1F3 |
| | SEQ ID | ILQGTGFAY | |
| | NO: 298 | | |
| V_{L}-CDR1 | SEQ ID | KSSQSLLYS | V_{L}-CDR1 sequence |
| | NO: 199 | SNQKNYLA | of 7B1F3 |
| | SEQ ID | RSSQSLLYS | |
| | NO: 295 | SNQKNYLA | |
| V_{L}-CDR2 | SEQ ID | WASTRES | V_{L}-CDR2 sequence |
| | NO: 222 | | of 7B1F3 |
| V_{L}-CDR3 | SEQ ID | QQYYSFPLT | V_{L}-CDR3 sequence |
| | NO: 223 | | of 7B1F3 |

In some preferred embodiments, the binding molecule of the first aspect of the present invention comprises one, two, three, four, five or six complementarity determining regions (CDRs) having binding specificity to the first epitope within a polypeptide consisting of the amino acid sequence of SEQ ID NO: 177 corresponding to any one, two, three, four, five or all six of the CDR sequences of antibody 2F5B11 as defined by SEQ ID NOs: 242, 243, 244, 199, 245 and 246, respectively, and/or a functional variant of any one or more of said CDR sequences of antibody 2F5B11, and optionally wherein the binding molecule is selected from an antibody and a CAR.

The sequences, and identities of the CDR sequences of antibody 2F5B11 as defined by SEQ ID NOs: 242, 243, 244, 199, 245 and 246 are as follows:

| | | |
|---|---|---|
| SEQ ID | SYWIH | V_{H}-CDR1 sequence |
| NO: 242 | | of 2F5B11 |
| SEQ ID | YINPNTAYTEFNQKFMD | V_{H}-CDR2 sequence |
| NO: 243 | | of 2F5B11 |
| SEQ ID | LRSDRTGFAY | V_{H}-CDR3 sequence |
| NO: 244 | | of 2F5B11 |
| SEQ ID | KSSQSLLYSSNQKNYLA | V_{L}-CDR1 sequence |
| NO: 199 | | of 2F5B11 |
| SEQ ID | WASTREY | V_{L}-CDR2 sequence |
| NO: 245 | | of 2F5B11 |
| SEQ ID | QQYYSFPLT | V_{L}-CDR3 sequence |
| NO: 246 | | of 2F5B11 |

In some preferred embodiments, the binding molecule of the first aspect of the present invention further comprises (and/or a second binding molecule that can be used and/or formulated in conjunction with a binding molecule of the first aspect of the present invention, comprises) one, two, three, four, five or six complementarity determining regions (CDRs) having binding specificity to the second epitope within ECD3 of a US28 protein of HCMV, corresponding to any one, two, three, four, five or all six of the CDR sequences of antibody 1D3 as defined by SEQ ID NOs: 8, 9, 10, 14, 15 and 16, respectively, and/or a functional variant of any one or more of said CDR sequences of antibody 1D3, and optionally wherein the or each binding molecule is selected from an antibody and a CAR.

The sequences, and identities of the CDR sequences of antibody 1D3 as defined by SEQ ID NOs: 8, 9, 10, 14, 15 and 16 are as follows:

| | | |
|---|---|---|
| SEQ ID | GFTFTDYY | V_{H}-CDR1 sequence |
| NO: 8 | | of 1D3 |
| SEQ ID | IRSKANGYTT | V_{H}-CDR2 sequence |
| NO: 9 | | of 1D3 |
| SEQ ID | ARDERRTAWLAY | V_{H}-CDR3 sequence |
| NO: 10 | | of 1D3 |
| SEQ ID | QSIVHSNGNTY | V_{L}-CDR1 sequence |
| NO: 14 | | of 1D3 |
| SEQ ID | KVS | V_{L}-CDR2 sequence |
| NO: 15 | | of 1D3 |
| SEQ ID | FQGSHVPTWT | V_{L}-CDR3 sequence |
| NO: 16 | | of 1D3 |

In another embodiment, the binding molecule of the first aspect of the present invention further comprises (and/or a second binding molecule that can be used and/or formulated in conjunction with a binding molecule of the first aspect of the present invention, comprises) one, two, three, four, five or six complementarity determining regions (CDRs) having binding specificity to the second epitope within ECD3 of a US28 protein of HCMV, corresponding to any one, two, three, four, five or all six of the CDR sequences of antibody 1C10 as defined by SEQ ID NOs: 112, 113, 114, 117, 83, 118, respectively, and/or a functional variant of any one or more of said CDR sequences of antibody 1C10, and optionally wherein the or each binding molecule is selected from an antibody and a CAR.

The sequences, and identities of the CDR sequences of antibody 1C10 as defined by SEQ ID NOs: 112, 113, 114, 117, 83, 118 are as follows:

| | | |
|---|---|---|
| SEQ ID | SHALS | V_{H}-CDR1 sequence |
| NO: 112 | | of 1C10 |
| SEQ ID | SISSRGRTYYPDSVKG | V_{H}-CDR2 sequence |
| NO: 113 | | of 1C10 |
| SEQ ID | GGTHYSYGNGFDF | V_{H}-CDR3 sequence |
| NO: 114 | | of 1C10 |
| SEQ ID | SVSSSVSYMH | V_{L}-CDR1 sequence |
| NO: 117 | | of 1C10 |
| SEQ ID | DTSKLAS | V_{L}-CDR2 sequence |
| NO: 83 | | of 1C10 |
| SEQ ID | QQWSNNPPIT | V_{L}-CDR3 sequence |
| NO: 118 | | of 1C10 |

In another embodiment, the binding molecule of the first aspect of the present invention further comprises (and/or a second binding molecule that can be used and/or formulated in conjunction with a binding molecule of the first aspect of the present invention, comprises) one, two, three, four, five or six complementarity determining regions (CDRs) having binding specificity to the second epitope within ECD3 of a US28 protein of HCMV, corresponding to any one, two, three, four, five or all six of the CDR sequences of antibody 1A10 as defined by SEQ ID NOs: 112, 113, 114, 117, 83, 118, respectively, and/or a functional variant of any one or more of said CDR sequences of antibody 1A10, and optionally wherein the or each binding molecule is selected from an antibody and a CAR.

The sequences, and identities of the CDR sequences of antibody 1A10 as defined by SEQ ID NOs: 112, 113, 114, 117, 83, 118 are as follows:

| | | |
|---|---|---|
| SEQ ID | SHALS | V_{H}-CDR1 sequence |
| NO: 112 | | of 1A10 |
| SEQ ID | SISSRGRTYYPDSVKG | V_{H}-CDR2 sequence |
| NO: 113 | | of 1A10 |
| SEQ ID | GGTHYSYGNGFDF | V_{H}-CDR3 sequence |
| NO: 114 | | of 1A10 |
| SEQ ID | SVSSSVSYMH | V_{L}-CDR1 sequence |
| NO: 117 | | of 1A10 |
| SEQ ID | DTSKLAS | V_{L}-CDR2 sequence |
| NO: 83 | | of 1A10 |
| SEQ ID | QQWSNNPPIT | V_{L}-CDR3 sequence |
| NO: 118 | | of 1A10 |

In another embodiment, the binding molecule of the first aspect of the present invention further comprises (and/or a second binding molecule that can be used and/or formulated in conjunction with a binding molecule of the first aspect of the present invention, comprises) one, two, three, four, five or six complementarity determining regions (CDRs) having binding specificity to the second epitope within ECD3 of a US28 protein of HCMV, corresponding to any one, two, three, four, five or all six of the CDR sequences of antibody 1G9 as defined by SEQ ID NOs: 76, 77, 78, 82, 83 and 84, respectively, and/or a functional variant of any one or more of said CDR sequences of antibody 1G9, and optionally wherein the or each binding molecule is selected from an antibody and a CAR.

The sequences, and identities of the CDR sequences of antibody 1G9 as defined by SEQ ID NOs: 76, 77, 78, 82, 83 and 84 are as follows:

| | | |
|---|---|---|
| SEQ ID | SYAMS | V_{H}-CDR1 sequence |
| NO: 76 | | of 1G9 |
| SEQ ID | SISSGGSTYYPDSVKG | V_{H}-CDR2 sequence |
| NO: 77 | | of 1G9 |
| SEQ ID | GGSTMITTGLGFAY | V_{H}-CDR3 sequence |
| NO: 78 | | of 1G9 |
| SEQ ID | SASSSVSYMH | V_{L}-CDR1 sequence |
| NO: 82 | | of 1G9 |
| SEQ ID | DTSKLAS | V_{L}-CDR2 sequence |
| NO: 83 | | of 1G9 |
| SEQ ID | QQWSSNPPLT | V_{L}-CDR3 sequence |
| NO: 84 | | of 1G9 |

In another embodiment, the binding molecule of the first aspect of the present invention further comprises (and/or a second binding molecule that can be used and/or formulated in conjunction with a binding molecule of the first aspect of the present invention, comprises) one, two, three, four, five or six complementarity determining regions (CDRs) having binding specificity to the second epitope within ECD3 of a US28 protein of HCMV, corresponding to any one, two, three, four, five or all six of the CDR sequences of antibody IE8 as defined by SEQ ID NOs: 76, 95, 96, 82, 99 and 100, respectively, and/or a functional variant of any one or more of said CDR sequences of antibody 1E8, and optionally wherein the or each binding molecule is selected from an antibody and a CAR.

The sequences, and identities of the CDR sequences of antibody 1E8 as defined by SEQ ID NOs: 76, 95, 96, 82, 99 and 100 are as follows:

| | | |
|---|---|---|
| SEQ ID | SYAMS | V_{H}-CDR1 sequence |
| NO: 76 | | of 1E8 |
| SEQ ID | SISSGGRTYYPDSVKG | V_{H}-CDR2 sequence |
| NO: 95 | | of 1E8 |
| SEQ ID | GGTRHSYGNGFDY | V_{H}-CDR3 sequence |
| NO: 96 | | of 1E8 |
| SEQ ID | SASSSVSYMH | V_{L}-CDR1 sequence |
| NO: 82 | | of 1E8 |
| SEQ ID | DSSKLAS | V_{L}-CDR2 sequence |
| NO: 99 | | of 1E8 |
| SEQ ID | QQWTSNPPIT | VL-CDR3 sequence |
| NO: 100 | | of 1E8 |

In other embodiments, the binding molecule of the first aspect of the present invention comprises one, two, three, four, five or six complementarity determining regions (CDRs) having binding specificity to the first epitope within a polypeptide consisting of the amino acid sequence of SEQ ID NO: 177, corresponding to any one, two, three, four, five or all six of the CDR sequences (and/or a functional variant of any one or more of said CDR sequences) of any other antibody having binding specificity (and preferably a strain agnostic binding specificity) to an epitope within SEQ ID NO: 177 of the ECD1 of US28, such as 4A11A11, 2G2B3, 5D6H11, 9G3B9, 5E8F7, 6G3D6 and/or 5E1E4; and/or optionally further comprises one, two, three, four, five or six complementarity determining regions (CDRs) having binding specificity to the second epitope within ECD3 of a US28 protein of HCMV, corresponding to any one, two, three, four, five or all six of the CDR sequences (and/or a functional variant of any one or more of said CDR sequences) of any other antibody having binding specificity (and preferably a strain agnostic binding specificity) to the ECD3 of US28, such as either of antibodies 13-5C6-1B5 and 14-1H3-1A6 as described herein, and/or any antibody that is obtained by the method of obtaining further antibodies having binding specificity to an epitope within extracellular domain 3 (ECD3) of a US28 protein of HCMV), as described above.

It is appreciated that molecules containing three or fewer CDR regions (in some cases just a single CDR or a part thereof) can be capable of retaining the antigen-binding activity of the antibody from which the CDR(s) are derived. For example, Gao et al (1994, J Biol Chem 269: 32389-93) describe a whole V_{L} chain (including all three CDRs) having high affinity for its substrate.

Molecules containing two CDR regions are described, for example, by Vaughan & Sollazzo (2001, Combinatorial Chenmistry & High Throughput Screening, 4: 417-430). On page 418 (right column—3 Our Strategy for Design) a minibody including only the H1 and H2 CDR hypervariable regions interspersed within framework regions is described. The minibody is described as being capable of binding to a target. Pessi et al (1993, Nature, 362: 367-9) and Bianchi et al (1994, J. MoL. Biol., 236: 649-59) are referenced by Vaughan & Sollazzo and describe the H1 and H2 minibody and its properties in more detail. Qiu et al (2007, Nature Biotechnology, 25:921-9) demonstrate that a molecule consisting of two linked CDRs are capable of binding antigen (abstract and page 926, right-hand column). Quiocho (1993, Nature, 362: 293-4) provides a summary of the Pessi et alt “minibody” technology. Ladner (2007, Nature Biotechnology, 25:875-7) reviews the Qiu et al. article and comments that molecules containing two CDRs are capable of retaining antigen-binding activity (page 875, right-hand column).

Molecules containing a single CDR region are described, for example, by Laune et al (1997, JBC, 272: 30937-44) who demonstrate that a range of hexapeptides derived from a CDR display antigen-binding activity (abstract) and note that synthetic peptides of a complete, single, CDR display strong binding activity (page 30942, right-hand column). Monnet et al (1999, JBC, 274: 3789-96) show that a range of 12-mer peptides and associated framework regions have antigen-binding activity (abstract) and comment that a CDR3-like peptide alone is capable of binding antigen (page 3785, left-hand column). Heap et al (2005, J. Gen. Virol., 86: 1791-1800) report that a “micro-antibody” (a molecule containing a single CDR) is capable of binding antigen (abstract and page 1791, left-hand column) and shows that a cyclic peptide from an anti-HIV antibody has antigen-binding activity and function. Nicaise et al (2004, Protein Science, 13:1882-91) show that a single CDR can confer antigen-binding activity and affinity for its lysozyme antigen.

Where functional variants of particular CDR sequences of an antibody are mentioned, it will be appreciated that one or more of the CDRs in the antibody as defined may be varied. Thus, where the antibody is defined as comprising light chain or heavy chain CDRs (e.g. CDRs 1-3), each having a particular sequence, up to one, two, or three of those particular sequences may be varied. Similarly, where the antibody is defined as comprising light chain and heavy chain CDRs (e.g. six CDRs), each having a particular sequence, up to one, two, three, four, five, or all six of those particular sequences may be varied. The functional variants are typically conservative amino acid substitutions as described further below and/or can include amino acid deletions and/or insertions. Alternatively, or additionally, the functional variants may be the humanised variants described herein.

For example, the V_{H}-CDR1 sequence of 1D3 is an 8-amino acid sequence GFTFTDYY (SEQ ID NO: 8). A functional variant thereof may include one or more (e.g. 1, 2, 3, 4, 5, 6, 7 or 8) conservative amino acid substitutions as described further below and/or can include one or more (e.g. 1, 2, 3, 4, or 5) amino acid deletions and/or one or more amino acid insertions. The V_{H}-CDR1 sequences of 4H3C3, 7B1F3, 2F5B11, 1C10, 1A10, 1G9 and 1E8 are 5-amino acid sequences (SEQ ID NOs: 196, 219, 242, 112, 112, 138, 76 and 76, respectively). Functional variants thereof may include one or more (e.g. 1, 2, 3, 4 or 5) conservative amino acid substitutions as described further below and/or can include one or more (e.g. 1 or 2) amino acid deletions and/or one or more amino acid insertions.

The V_{H}-CDR2 sequence of 1D3 is a 10-amino acid sequence IRSKANGYTT (SEQ ID NO: 9). A functional variant thereof may include one or more (e.g. 1, 2, 3, 4, 5, 6, 7, 8, 9 or 10) conservative amino acid substitutions as described further below and/or can include one or more (e.g. 1, 2, 3, 4, 5, 6 or 7) amino acid deletions and/or one or more amino acid insertions. The V_{H}-CDR2 sequences of 1C10, 1A10, 1G9 and 1E8 are 16-amino acid sequences (SEQ ID NOs: 113, 113, 77 and 95, respectively). Functional variants thereof may include one or more (e.g. 1, 2, 3, 4, 5, 6, 7, 8, 9, 10, 11, 12, 13, 14, 15 or 16) conservative amino acid substitutions as described further below and/or can include one or more (e.g. 1, 2, 3, 4, 5, 6, 7, 8, 9, 10, 11, 12 or 13) amino acid deletions and/or one or more amino acid insertions. The V_{H}-CDR2 sequences of 4H3C3, 7B1F3 and 2F5B11 are 17-amino acid sequences (SEQ ID NOs: 197 (or humanised variants SEQ ID NOs: 293 and 294), 220 (or humanised variants SEQ ID NO: 296 and 297) and 243, respectively). Functional variants thereof may include one or more (e.g. 1, 2, 3, 4, 5, 6, 7, 8, 9, 10, 11, 12, 13, 14, 15, 16 or 17) conservative amino acid substitutions as described further below and/or can include one or more (e.g. 1, 2, 3, 4, 5, 6, 7, 8, 9, 10, 11, 12, 13 or 14) amino acid deletions and/or one or more amino acid insertions.

The V_{H}-CDR3 sequence of 1D3 is a 12-amino acid sequence ARDERRTAWLAY (SEQ ID NO: 10). A functional variant thereof may include one or more (e.g. 1, 2, 3, 4, 5, 6, 7, 8, 9, 10, 11 or 12) conservative amino acid substitutions as described further below and/or can include one or more (e.g. 1, 2, 3, 4, 5, 6, 7, 8 or 9) amino acid deletions and/or one or more amino acid insertions. The V_{H}-CDR3 sequence of 1G9 is a 14-amino acid sequence (SEQ ID NO: 78). A functional variant thereof may include one or more (e.g. 1, 2, 3, 4, 5, 6, 7, 8, 9, 1.0, 1.1, 1.2, 1.3 or 14) conservative amino acid substitutions as described further below and/or can include one or more (e.g. 1, 2, 3, 4, 5, 6, 7, 8, 9, 10 or 11) amino acid deletions and/or one or more amino acid insertions. The V_{H}-CDR3 sequences of 1C10, 1A10 and 1E8 are 13-amino acid sequences (SEQ ID NOs: 114, 114 and 96, respectively). Functional variants thereof may include one or more (e.g. 1, 2, 3, 4, 5, 6, 7, 8, 9, 10, 11, 12 or 13) conservative amino acid substitutions as described further below and/or can include one or more (e.g. 1, 2, 3, 4, 5, 6, 7, 8, 9 or 10) amino acid deletions and/or one or more amino acid insertions. The W-CDR3 sequences of 4H3C3 and 2F5B11 are 10-amino acid sequences (SEQ ID NOs: 198 and 244, respectively). Functional variants thereof may include one or more (e.g. 1, 2, 3, 4, 5, 6, 7, 8, 9 or 10) conservative amino acid substitutions as described further below and/or can include one or more (e.g. 1, 2, 3, 4, 5, 6 or 7) amino acid deletions and/or one or more amino acid insertions. The V_{H}-CDR3 sequence of 7B1F3 is a 9-amino acid sequence (SEQ ID NOs: 221 or humanised variant SEQ ID NO: 298). Functional variants thereof may include one or more (e.g. 1, 2, 3, 4, 5, 6, 7, 8 or 9) conservative amino acid substitutions as described further below and/or can include one or more (e.g. 1, 2, 3, 4, 5 or 6) amino acid deletions and/or one or more amino acid insertions.

The V_{L}-CDR1 sequence of 1D3 is an 11-amino acid sequence QSIVHSNGNTY (SEQ ID NO: 14). A functional variant thereof may include one or more (e.g. 1, 2, 3, 4, 5, 6, 7, 8, 9 or 10) conservative amino acid substitutions as described further below and/or can include one or more (e.g. 1, 2, 3, 4, 5, 6, 7 or 8) amino acid deletions and/or one or more amino acid insertions. The V_-CDR1 sequences of 1C10, 1A10, 1G9 and 1E8 are 10-amino acid sequences (SEQ ID NOs: 117, 117, 82 and 82, respectively). Functional variants thereof may include one or more (e.g. 1, 2, 3, 4, 5, 6, 7, 8, 9 or 10) conservative amino acid substitutions as described further below and/or can include one or more (e.g. 1, 2, 3, 4, 5, 6 or 7) amino acid deletions and/or one or more amino acid insertions. The V_{L}-CDR1 sequences of 4H3C3, 7B1F3 and 2F5B11 are 17-amino acid sequences (SEQ ID NOs: 199 (or humanised variant SEQ ID NO: 295), 199 (or humanised variant SEQ ID NO: 295) and 199, respectively). Functional variants thereof may include one or more (e.g. 1, 2, 3, 4, 5, 6, 7, 8, 9, 10, 11, 12, 13, 14, 15, 16 or 17) conservative amino acid substitutions as described further below and/or can include one or more (e.g. 1, 2, 3, 4, 5, 6, 7, 8, 9, 10, 11, 12, 13 or 14) amino acid deletions and/or one or more amino acid insertions.

The V_{L}-CDR2 sequence of 1D3 is a 3-amino acid sequence KVS (SEQ ID NO: 15). A functional variant thereof may include one or more (e.g. 1, 2, 3) conservative amino acid substitutions as described further below and/or can include one or more amino acid deletions and/or one or more amino acid insertions. The V_{L}-CDR2 sequences of 4H3C3, 7B1F3, 2F5B11, 1C10, 1A10, 1G9 and IE8 are 7-amino acid sequences (SEQ ID NOs: 200 (or humanised variant SEQ ID NO: 222), 222, 245, 83, 83, 145, 83 and 99, respectively). Functional variants thereof may include one or more (e.g. 1, 2, 3, 4, 5, 6 or 7) conservative amino acid substitutions as described further below and/or can include one or more (e.g. 1, 2, 3 or 4) amino acid deletions and/or one or more amino acid insertions.

The V_{L}-CDR3 sequence of 1D3 is a 10-amino acid sequence FQGSHVPTWT (SEQ ID NO: 16). A functional variant thereof may include one or more (e.g. 1, 2, 3, 4, 5, 6, 7, 8, 9 or 10) conservative amino acid substitutions as described further below and/or can include one or more (e.g. 1, 2, 3, 4, 5, 6 or 7) amino acid deletions and/or one or more amino acid insertions. The V_{L}-CDR3 sequences of 1C10, 1A10, 1G9 and 1ES are 10-amino acid sequences (SEQ ID NOs: 118, 118, 84 and 100, respectively). A functional variant thereof may include one or more (e.g. 1, 2, 3, 4, 5, 6, 7, 8, 9 or 10) conservative amino acid substitutions as described further below and/or can include one or more (e.g. 1, 2, 3, 4, 5, 6 or 7) amino acid deletions and/or one or more amino acid insertions. The V_{L}-CDR3 sequences of 4H3C3, 7B1F3 and 2F5B11 are 9-amino acid sequences (SEQ ID NOs: 201, 223 and 246, respectively). Functional variants thereof may include one or more (e.g. 1, 2, 3, 4, 5, 6, 7, 8 or 9) conservative amino acid substitutions as described further below and/or can include one or more (e.g. 1, 2, 3, 4, 5 or 6) amino acid deletions and/or one or more amino acid insertions. Functional variants of one or more of said CDRs can be created by any of the various processes well known in the art. For example, the binding properties of a binding molecule that comprises 1, 2, 3, 4, 5 or 6 CDRs can be developed by a maturation process. For example, the binding affinity provided the CDRs of a binding molecule can be modified (increased, or reduced) by maturation steps; and/or the binding specificity provided the CDRs of a binding molecule can be modified (in general, increased) by maturation steps. It is to be understood that high, or increased, levels of binding affinity to US28 may not always be desirable, in particular if this comes at the expense of unacceptably high levels of binding affinity to off-targets, such as healthy human cells. Binding molecules, including but not limited to CARs and CAR-expressing cells, for example, may benefit from lower levels of binding affinity, but will generally always benefit from optimised levels of binding specificity.

In one example of a maturation process, a monovalent display phagemid system may be used to modify the avidity effects during antigen-binding screening. Two alternative or combined methods, untargeted mutagenesis and oligonucleotide-directed mutagenesis, can be employed to construct random or defined sublibraries to introduce a large number of mutants of the original binding molecule. The binding molecules that bind to US28-expressing cells, and/or to HCMV-infected cells, with the desired modified properties (such as increased specificity and/or increased or decreased affinity) are then selected by modifying the screening conditions, such as in an assay for stringency.

Binding molecule of the first aspect of the present invention that comprise one or more sequences that provide binding specificity to a first epitope within a polypeptide consisting of the amino acid sequence of TDVLNQSKPVTL (SEQ ID NO: 177), wherein said one or more sequences comprise functional variants of any of the one, two, three, four, five or all six of the CDR sequences of antibody:
(a) 4H3C3 as defined by SEQ ID NOs: 196, 197, 198, 199, 200 and 201, respectively;
(b) 7B1F3 as defined by SEQ ID NOs: 219, 220, 221, 199, 222 and 223, respectively; and/or
(c) 2F5B11 as defined by SEQ ID NOs: 242, 243, 244, 199, 245 and 246, respectively;
(d) 4H3C3 humanised variant as defined by SEQ ID NOs: 196; 293 or 294; 198; 199 or 295; 200 or 222; and 201, respectively;
(e) 7B1F3 humanised variant as defined by SEQ ID NOs: 219; 296 or 297; 221 or 298; 199 or 295; 222; and 223, respectively;
and preferably possess one or more binding properties to a peptide having the sequence of SEQ ID NO: 177 and/or to the US28 protein that are similar or substantially equivalent to the binding properties of 4H3C3, 7B1F3 and/or 2F5B11, when tested under the same conditions as 4H3C3, 7B1F3 and/or 2F5B11, respectively, as further described above.

Optionally the binding molecule of first aspect of the present invention, for example as described in the preceding paragraph, further comprises one or more sequences that have binding specificity to a second epitope within extracellular domain 3 (ECD3) of a US28 protein of HCMV, wherein said one or more sequences comprise (and/or a second binding molecule that can be used and/or formulated in conjunction with a binding molecule of the first aspect of the present invention, comprises) functional variants of any of the one, two, three, four, five or all six of the CDR sequences of antibody:
(f) 1D3 as defined by SEQ ID NOs: 8, 9, 10, 14, 15 and 16, respectively;
(g) 1C10 as defined by SEQ ID NOs: 112, 113, 114, 117, 83 and 118, respectively;
(h) 1A10 as defined by SEQ ID NOs: 112, 113, 114, 117, 83 and 118, respectively;
(i) IG9 as defined by SEQ ID NOs: 76, 77, 78, 82, 83 and 84, respectively; and/or
(j) 1E8 as defined by SEQ ID NOs: 76, 95, 96, 82, 99 and 100, respectively;
and preferably possess one or more binding properties to a peptide having the sequence of either or both of the 4N and/or 4D variants of ECD3 of the US28 protein of HCMV, and/or to the US28 protein having either or both of the 4D and/or 4N variant ECD3 sequence, that are similar or substantially equivalent to the binding properties of 1D3, 1C10, 1A10, 1G9 and/or 1E8, when tested under the same conditions as 1D3, 1C10, 1A10, 1G9 and/or 1E8, respectively, as further described above.

In one embodiment, the binding molecule of the first aspect of the present invention may comprise:
(a) one, two, or all three, of the CDR 1, 2, and 3, sequences of the variable heavy chain (V_{H}) of antibody:
   i. 4H3C3, as defined by SEQ ID NOs: 196, 197 and 198, respectively (and/or a functional variant of any one, two or three of said CDR sequences);
   ii. 7B1F3, as defined by SEQ ID NOs: 219, 220 and 221, respectively (and/or a functional variant of any one, two or three of said CDR sequences); and/or
   iii. 2F5B11, as defined by SEQ ID NOs: 242, 243 and 244, respectively (and/or a functional variant of any one, two or three of said CDR sequences);
      and optionally further comprise:
   iv. 1D3, as defined by SEQ ID NOs: 8, 9, and 10, respectively (and/or a functional variant of any one, two or three of said CDR sequences);
   v. 1C10, as defined by SEQ ID NOs: 112, 113, and 114, respectively (and/or a functional variant of any one, two or three of said CDR sequences);
   vi. 1A10, as defined by SEQ ID NOs: 112, 113, and 114, respectively (and/or a functional variant of any one, two or three of said CDR sequences);
   vii. 1G9, as defined by SEQ ID NOs: 76, 77, and 78, respectively (and/or a functional variant of any one, two or three of said CDR sequences); and/or
   viii. 1E8, as defined by SEQ ID NOs: 76, 95, and 96, respectively (and/or a functional variant of any one, two or three of said CDR sequences);
and/or
(b) one, two, or all three, of the CDR 1, 2, and 3, sequences of the variable light chain (V_{L}) of antibody:
   i. 4H3C3, as defined by SEQ ID NOs: 199, 200 and 201, respectively (and/or a functional variant of any one, two or three of said CDR sequences);
   ii. 7B1F3, as defined by SEQ ID NOs: 199, 222 and 223, respectively (and/or a functional variant of any one, two or three of said CDR sequences); and/or
   iii. 2F5B11, as defined by SEQ ID NOs: 199, 245 and 246, respectively (and/or a functional variant of any one, two or three of said CDR sequences);
      and optionally further comprise:
   iv. 1D3, as defined by SEQ ID NOs: 14, 15, and 16, respectively (and/or a functional variant of any one, two or three of said CDR sequences);
   v. 1C10, as defined by SEQ ID NOs: 117, 83, and 118, respectively (and/or a functional variant of any one, two or three of said CDR sequences);
   vi. 1A10, as defined by SEQ ID NOs: 117, 83, and 118, respectively (and/or a functional variant of any one, two or three of said CDR sequences);
   vii. 1G9, as defined by SEQ ID NOs: 82, 83, and 84, respectively (and/or a functional variant of any one, two or three of said CDR sequences); and/or
   viii. 1E8, as defined by SEQ ID NOs: 82, 99, and 100, respectively (and/or a functional variant of any one, two or three of said CDR sequences).

Additionally, or alternatively, a binding molecule of the first aspect of the present invention may comprise:
(a) at least one variable heavy chain (V_{H}) polypeptide that comprises CDR 1, 2, and 3 sequences having the sequences of:
   i. SEQ ID NOs: 196, 197, and 198, respectively (and/or a functional variant of any one, two or three of said CDR sequences), and optionally wherein the at least one variable heavy chain (V_{H}) polypeptide comprises, consists essentially of, or consists of, the sequence of SEQ ID NO: 187 (or a functional variant thereof);
ii. SEQ ID NOs: 219, 220, and 221, respectively (and/or a functional variant of any one, two or three of said CDR sequences), and optionally wherein the at least one variable heavy chain (V_{H}) polypeptide comprises, consists essentially of, or consists of, the sequence of SEQ ID NO: 211 (or a functional variant thereof); and/or
   iii. SEQ ID NOs: 242, 243, and 244, respectively (and/or a functional variant of any one, two or three of said CDR sequences), and optionally wherein the at least one variable heavy chain (V_{H}) polypeptide comprises, consists essentially of, or consists of, the sequence of SEQ ID NO: 233 (or a functional variant thereof);
      and optionally of:
   iv. SEQ ID NOs: 8, 9, and 10, respectively (and/or a functional variant of any one, two or three of said CDR sequences), and optionally wherein the at least one variable heavy chain (V_{H}) polypeptide comprises, consists essentially of, or consists of, the sequence of SEQ ID NO: 12 (or a functional variant thereof);
   v. SEQ ID NOs: 112, 113, and 114, respectively (and/or a functional variant of any one, two or three of said CDR sequences), and optionally wherein the at least one variable heavy chain (V) polypeptide comprises, consists essentially of, or consists of, the sequence of SEQ ID NO: 104 (or a functional variant thereof);
   vi. SEQ ID NOs: 112, 113, and 114, respectively (and/or a functional variant of any one, two or three of said CDR sequences), and optionally wherein the at least one variable heavy chain (V) polypeptide comprises, consists essentially of, or consists of, the sequence of SEQ ID NO: 122 (or a functional variant thereof);
   vii. SEQ ID NOs: 76, 77, and 78, respectively (and/or a functional variant of any one, two or three of said CDR sequences), and optionally wherein the at least one variable heavy chain (V_{H}) polypeptide comprises, consists essentially of, or consists of, the sequence of SEQ ID NO: 68 (or a functional variant thereof); and/or
   viii. SEQ ID NOs: 76, 95, and 96, respectively (and/or a functional variant of any one, two or three of said CDR sequences), and optionally wherein the at least one variable heavy chain (V_{H}) polypeptide comprises, consists essentially of, or consists of, the sequence of SEQ ID NO: 88 (or a functional variant thereof);
and/or
(b) at least one variable light chain (V_{L}) polypeptide that comprises CDR 1, 2 and 3 sequences having the sequences of:
   i. SEQ ID NOs: 199, 200, and 201, respectively (and/or a functional variant of any one, two or three of said CDR sequences), and optionally wherein the at least one variable heavy chain (V_{L}) polypeptide comprises, consists essentially of, or consists of, the sequence of SEQ ID NO: 189 (or a functional variant thereof);
   ii. SEQ ID NOs: 199, 222, and 223, respectively (and/or a functional variant of any one, two or three of said CDR sequences), and optionally wherein the at least one variable heavy chain (V_{L}) polypeptide comprises, consists essentially of, or consists of, the sequence of SEQ ID NO: 213 (or a functional variant thereof); and/or
   iii. SEQ ID NOs: 199, 245, and 246, respectively (and/or a functional variant of any one, two or three of said CDR sequences), and optionally wherein the at least one variable heavy chain (V_{L}) polypeptide comprises, consists essentially of, or consists of, the sequence of SEQ ID NO: 235 (or a functional variant thereof);
      and optionally of:
   iv. SEQ ID NOs: 14, 15, and 16, respectively (and/or a functional variant of any one, two or three of said CDR sequences), and optionally wherein the variable light chain (V_{L}) polypeptide comprises, consists essentially of, or consists of, the sequence of SEQ ID NO: 18;
   v. SEQ ID NOs: 117, 83, and 118, respectively (and/or a functional variant of any one, two or three of said CDR sequences), and optionally wherein the variable light chain (V_{L}) polypeptide comprises, consists essentially of, or consists of, the sequence of SEQ ID NO: 108;
   vi. SEQ ID NOs: 117, 83, and 118, respectively (and/or a functional variant of any one, two or three of said CDR sequences), and optionally wherein the variable light chain (V_{L}) polypeptide comprises, consists essentially of, or consists of, the sequence of SEQ ID NO: 126;
   vii. SEQ ID NOs: 82, 83, and 84, respectively (and/or a functional variant of any one, two or three of said CDR sequences), and optionally wherein the variable light chain (V_{L}) polypeptide comprises, consists essentially of, or consists of, the sequence of SEQ ID NO: 72; and/or
   viii. SEQ ID NOs: 82, 99, and 100, respectively (and/or a functional variant of any one, two or three of said CDR sequences), and optionally wherein the variable light chain (V_{L}) polypeptide comprises, consists essentially of, or consists of, the sequence of SEQ ID NO: 92.

The sequences, and identities of the variable heavy chain (V_{H}) variable light chain (V_{L}) polypeptide sequence of antibody 4H3C3 as defined by SEQ ID NOs: 187 and 189, respectively are as follows:

| | | |
|---|---|---|
| SEQ ID NO: | QVQLQQSGAE LAKPGASVKM SCKASGYTFT | The mature form of V_{H} |
| 187 | SYWMHWVKQR PGQGLEWIGY INPSTGYAEY | sequence of 4H303, without |
| | NQKFKDKATL TADKSSSTAY MQLSSLTSED | the leader sequence |
| | SAVYYCARLR FGSSGFAYWG QGTLVTVSS | |
| SEQ ID NO: | QIVLTQSPSS LAVSVGEKVT MSCKSSQSLL | The mature form of V_{L} |
| 189 | YSSNQKNYLA WYQQKPGQSP KLLIYWASTW | sequence of 4H3C3, without |
| | ESGVPDRFTG SGSGTDFTLT ISSVKAEDLA | the leader sequence |
| | VYYCQQYYSF PLTFGAGTKL EIK | |

The sequences, and identities of the humanised variable heavy chain (V_{H}) variable light chain (V_{L}) polypeptide sequence of antibody 4H3C3 may be as follows:

| | | |
|---|---|---|
| SEQ ID NO: | QVQLVQSGAEVKKPGASVKVSCKASGYTFTSYWMHWVRQAPG | Also referred to herein as |
| 256 | QGLEWIGYINPSTGYAEYNQKFQGRATLTADKSSSTAYMELS | 4H3C3 VH1. |
| | SLRSEDTAVYYCARLRFGSSGFAYWGQGTLVTVSS | |
| SEQ ID NO: | QVQLVQSGAEVKKPGASVKVSCKASGYTFTSYWMHWVRQAPG | Also referred to herein as |
| 257 | QGLEWMGYINPSTGYAEYNQKFQGRVTMTADKSTSTAYMELS | 4H3C3 VH2. |
| | SLRSEDTAVYYCARLREGSSGFAYWGQGTLVTVSS | |
| SEQ ID NO: | QVQLVQSGAEVKKPGASVKVSCKASGYTFTSYWMHWVRQAPG | Also referred to herein as |
| 258 | QGLEWIGYINPSTGYAEYNQKLQGRATLTADKSSSTAYMELR | 4H3C3 VH3. |
| | SLRSDDTAVYYCARLREGSSGFAYWGQGTLVTVSS | |
| SEQ ID NO: | QVQLVQSGAEVKKPGASVKVSCKASGYTFTSYWMHWVRQAPG | Also referred to herein as |
| 259 | QGLEWMGYINPSTGYAEYNQKLQGRVTMTADKSTSTAYMELR | 4H3C3 VH4. |
| | SLRSDDTAVYYCARLRFGSSGFAYWGQGTLVTVSS | |
| SEQ ID NO: | QIVLTQSPDSLAVSIGERATINCKSSQSLLYSSNQKNYLAWY | Also referred to herein as |
| 260 | QQKPGQPPKLLIYWASTWESGVPDRFSGSGSGTDFTLTISSL | 4H3C3 VL1. |
| | QAEDVAVYYCQQYYSFPLTFGGGTKLEIK | |
| SEQ ID NO: | DIVLTQSPDSLAVSIGERATINCKSSQSLLYSSNQKNYLAWY | Also referred to herein as |
| 261 | QQKPGQPPKLLIYWASTRESGVPDRFSGSGSGIDFILTISSL | 4H3C3 VL2. |
| | QAEDVAVYYCQQYYSEPLTFGGGTKLEIK | |
| SEQ ID NO: | QIVLTQSPLSLPVTPGEPASISCRSSQSLLYSSNQKNYLAWY | Also referred to herein as |
| 262 | LQKPGQSPQLLIYWASTWESGVPDRFSGSGSGTDFTLKISRV | 4H3C3 VL3. |
| | EAEDVGVYYCQQYYSFPLTFGGGTKLEIK | |
| SEQ ID NO: | DIVITQSPLSLPVTPGEPASISCRSSQSLLYSSNQKNYLAWY | Also referred to herein as |
| 263 | LQKPGQSPQLLIYWASTRESGVPDRFSGSGSGTDFTLKISRV | 4H3C3 VL4. |
| | EAEDVGVYYCQQYYSEPLTFGGGTKLEIK | |

The sequences, and identities of the variable heavy chain (V_{H}) variable light chain (V_{L}) polypeptide sequence of antibody 7B1F3 as defined by SEQ ID NOs: 211 and 213, respectively are as follows:

| | | |
|---|---|---|
| SEQ ID NO: | EVQLQQSGAE LAKPGASVKM SCKASGYTFT | The mature form of V_{H} |
| 211 | NYWMHWVKQR PGQGLEWIGY VNPRTGYTEY | sequence of 7B1F3, without |
| | NQKFKDKATL TADKSSRTAY MQLSSLTSED | the leader sequence |
| | SAVYSCARIL NGTGFAYWGQ GTLVTVSA | |
| SEQ ID NO: | DIVLTQSPSS LTVSVGEKFT MSCKSSQSLL | The mature form of V_{L} |
| 213 | YSSNQKNYLA WYQQKPGQSP KLLIYWASTR | sequence of 7B1F3, without |
| | ESGVPDRFTG SGSGTDFTLA ISSVKPEDLA | the leader sequence |
| | IYYCQQYYSF PLTFGAGTKL EIK | |

The sequences and identities of the humanised variable heavy chain (V_{H}) variable light chain (V_{L}) polypeptide sequence of antibody 7B1F3 may be as follows:

| | | |
|---|---|---|
| SEQ ID NO: | QVQLVQSGAEVKKPGASVKVSCKASGYTFTNYWMHWVRQAPG | Also referred to herein as |
| 264 | QGLEWIGYVNPRTGYTEYNQKFQGRATLTADKSSRTAYMELS | 7B1F3 VH1. |
| | SLRSEDTAVYSCARILNGTGFAYWGQGTLVTVSS | |
| SEQ ID NO: | QVQLVQSGAEVKKPGASVKVSCKASGYTFTNYWMHWVRQAPG | Also referred to herein as |
| 265 | QGLEWMGYVNPRTGYTEYNQKFQGRVTMTADKSTSTAYMELS | 7B1F3 VH2. |
| | SLRSEDTAVYYCARILNGTGFAYWGQGTLVTVSS | |
| SEQ ID NO: | QVQLVQSGAEVKKPGASVKVSCKASGYTFTNYWMHWVRQAPG | Also referred to herein as |
| 266 | QGLEWIGYVNPRTGYTEYNQQLQGRATLTADKSSRTAYMELR | 7B1F3 VH3. |
| | SLRSDDTAVYSCARILNGTGFAYWGQGTLVTVSS | |
| SEQ ID NO: | QVQLVQSGAEVKKPGASVKVSCKASGYTFTNYWMHWVRQAPG | Also referred to herein as |
| 267 | QGLEWMGYVNPRTGYTEYNQQLQGRVIMTADKSTSTAYMELR | 7B1F3 VH4. |
| | SLRSDDTAVYYCARIINGTGFAYWGQGTLVTVSS | |
| SEQ ID NO: | QVQLVQSGAEVKKPGASVKVSCKASGYTFTNYWMHWVRQAPG | Also referred to herein as |
| 270 | QGLEWIGYVNPRTGYTEYNQKFQGRATLTADKSSRTAYMELS | 7B1F3 VH5. |
| | SLRSEDTAVYSCARILQGTGFAYWGQGTLVTVSS | |
| SEQ ID NO: | QVQLVQSGAEVKKPGASVKVSCKASGYTFTNYWMHWVRQAPG | Also referred to herein as |
| 271 | QGLEWMGYVNPRTGYTEYNQKFQGRVTMTADKSTSTAYMELS | 7B1F3 VH6. |
| | SLRSEDTAVYYCARILQGTGFAYWGQGTLVTVSS | |
| SEQ ID NO: | DIVLTQSPDSLAVSIGERATINCKSSQSLLYSSNQKNYLAWY | Also referred to herein as |
| 268 | QQKPGQPPKLLIYWASTRESGVPDRFSGSGSGTDFILTISSL | 7B1F3 VL1. |
| | QAEDVATYYCQQYYSEPLTFGGGTKLEIK | |
| SEQ ID NO: | DIVLTQSPDSLAVSEGERATINCKSSQSLLYSSNQKNYLAWY | Also referred to herein as |
| 261 | QQKPGQPPKLLIYWASTRESGVPDRFSGSGSGTDFTLTISSL | 7B1F3 VL2. |
| | QAEDVAVYYCQQYYSFPLTFGGGTKLEIK | |
| SEQ ID NO: | DIVITQSPLSLPVTPGEPASISCRSSQSLLYSSNQKNYLAWY | Also referred to herein as |
| 269 | LQKPGQSPQLLIYWASTRESGVPDRFSGSGSGTDFTLKISRV | 7B1F3 VL3. |
| | EAEDVGIYYCQQYYSEPLTFGGGTKLEIK | |
| SEQ ID NO: | DIVLTQSPLSLPVTPGEPASISCRSSQSLLYSSNQKNYLAWY | Also referred to herein as |
| 263 | LQKPGQSPQLLIYWASTRESGVPDRFSGSGSGTDFTLKISRV | 7B1F3 VL4. |
| | EAEDVGVYYCQQYYSFPLTFGGGTKLEIK | |

The sequences, and identities of the variable heavy chain (V_{H}) variable light chain (V_{L}) polypeptide sequence of antibody 2F5B11 as defined by SEQ ID NOs: 233 and 235, respectively are as follows:

| | | |
|---|---|---|
| SEQ ID NO: | EVQLQESGAE LAKPGASVKM SCKASGYTFS | The mature form of V_{H} |
| 233 | SYWIHWIKQR PGQGLEWIGY INPNTAYTEF | sequence of 2F5B11, without |
| | NQKFMDKATL TADKSSSTAY MQLTSLTSED | the leader sequence |
| | SAVYYCARLR SDRTGFAYWG QGTLVTVSA | |
| SEQ ID NO: | QIVLTQSPSS LAVSVGEKVT MSCKSSQSLL | The mature form of V_{L} |
| 235 | YSSNQKNYLA WYQQKPGQSP KVLIYWASTR | sequence of 2F5B11, without |
| | EYGVPDRFTG SGSGTDFTLT ISSVKAEDLA | the leader sequence |
| | VYYCQQYYSF PLTFGAGTKL EIK | |

The sequences, and identities of the variable heavy chain (V_{H}) variable light chain (V_{L}) polypeptide sequence of antibody 1D3 as defined by SEQ ID NOs: 12 and 18, respectively are as follows:

| | | |
|---|---|---|
| SEQ ID NO: | EVKLVESGGG LVQPGGSLRL ACATSGFTFT | The mature form of V_{H} |
| 12 | DYYMSWVRQP PGKALEWLGF IRSKANGYTT | sequence of 1D3, |
| | EYSASVKGRF TISRDNSQSI LYLQMNTLRS | without the |
| | EDSATYYCAR DERRTAWLAY WGQGTLVTVS | leader sequence |
| | A | |
| SEQ ID NO: | DVLMTQTPLS LPVSLGDQAS ISCRSSQSIV | The mature form of V_{L} |
| 18 | HSNGNTYLDW YLQKPGQSPK LLIYKVSNRF | sequence of 1D3, |
| | SGVPDRFSGS GSGIDFILKI SRVEAEDLGV | without the |
| | YYCFQGSHVP TWIFGGGTKL EIK | leader sequence |

The sequences, and identities of the variable heavy chain (V_{H}) variable light chain (V_{L}) polypeptide sequence of antibody 1C10 as defined by SEQ ID NOs: 104 and

| | | |
|---|---|---|
| SEQ ID NO: | EVKLVESGGV LVKPGGSLKF SCAASGFTFS | The mature form of V_{H} |
| 104 | SHALSWVRQT PEKRLEWVAS ISSRGRTYYP | sequence of 1010, without |
| | DSVKGRFTVS RDNARNILYL QVSSLRSEDT | the leader sequence |
| | AMYYCTRGGT HYSYGNGFDF WGQGTTLTVS | |
| | S | |
| SEQ ID NO: | QIVLTQSPAI MSASPGEKVT MTCSVSSSVS | The mature form of V_{L} |
| 108 | YMHWYQQKSG TSPKRWIYDT SKLASGVPAR | sequence of 1010, without |
| | FSGSGSGTSY SLTISTMEAE DAATYYCQQW | the leader sequence |
| | SNNPPITFGA GTKLELK | |

The sequences, and identities of the variable heavy chain (V_{H}) variable light chain (V_{L}) polypeptide sequence of antibody 1A10 as defined by SEQ ID NOs: 122 and 126, respectively are as follows:

| | | |
|---|---|---|
| SEQ ID NO: | EVKLVESGGV LVKPGGSLKF SCAASGFTLS | The mature form of V_{H} |
| 122 | SHALSWVRQT PEKRLEWVAS ISSRGRTYYP | sequence of 1A10, without |
| | DSVKGRFTVS RDNARNILYL QVSSLRSEDT | the leader sequence |
| | AMYYCTRGGT HYSYGNGFDF WGQGTTLTVS | |
| | S | |
| SEQ ID NO: | QIVLTQSPAI MSASPGEKVT MICSVSSSVS | The mature form of V_{L} |
| 126 | YMHWYQQKSG TSPKRWIYDT SKLASGVPAR | sequence of 1A10, without |
| | FSGSGSGTSY SLTISTMEAE DAATYYCQQW | the leader sequence |
| | SNNPPITFGA GTKLELK | |

The sequences, and identities of the variable heavy chain (V_{H}) variable light chain (V_{L}) polypeptide sequence of antibody 1G9 as defined by SEQ ID NOs: 68 and 72, respectively are as follows:

| | | |
|---|---|---|
| SEQ ID NO: | EVKLVESGGG LVKPGGSLKL SCAASGFTFS | The mature form of V_{H} |
| 68 | SYAMSWVRQT PEKRLEWVAS ISSGGSTYYP | sequence of 1G9, without the |
| | DSVKGRFTIS RDNARNILYL QMSSLRSEDT | leader sequence |
| | AMYYCARGGS TMITTGLGFA YWGQGTLVTV | |
| | SA | |
| SEQ ID NO: | QIVLTQSPAI MSASPGEKVT MTCSASSSVS | The mature form of V_{L} |
| 72 | YMHWYQQKSG TSPKRWIYDT SKLASGVPAR | sequence of 1G9, without the |
| | FSGSGSGTSY SLTISSMEAE DAATYYCQQW | leader sequence |
| | SSNPPLTFGA GTKLELK | |

The sequences, and identities of the variable heavy chain (V_{H}) variable light chain (V_{L}) polypeptide sequence of antibody 1E8 as defined by SEQ ID NOs: 88 and 92, respectively are as follows:

| | | |
|---|---|---|
| SEQ ID NO: | EVKLVESGGD LVKPGGSLKL SCAASGFTFS | The mature form of V_{H} |
| 88 | SYAMSWVRQT PEKRLEWVAS ISSGGRTYYP | sequence of 1E8, without the |
| | DSVKGRFTIS RDNARNILYL QMSSLRSEDT | leader sequence |
| | AIYYCARGGT RHSYGNGFDY WGQGTTLTVS | |
| | S | |
| SEQ ID NO: | QIVLSQSPTI MSASPGEKVT MTCSASSSVS | The mature form of V_{L} |
| 92 | YMHWYQQKSG TSPKRWIYDS SKLASGVPAR | sequence of 1E8, without the |
| | FSGSGSGTSY SLTISSMEAE DAATYYCQQW | leader sequence |
| | TSNPPITFGA GTKLELK | |

In other embodiments, the binding molecule of the first aspect of the present invention comprises (a) at least one variable heavy chain (V) polypeptide that comprises CDR 1, 2, and 3 sequences of the V_{H} chain of another antibody having binding specificity (and preferably a strain agnostic binding specificity) to the first epitope within a polypeptide consisting of the amino acid sequence of SEQ ID NO: 177 in ECD1 (and optionally wherein the binding molecule also has binding specificity to a second epitope within ECD3) of US28 (and/or a functional variant of any one, two or three of said CDR sequences), and/or (b) at least one variable light chain (V_{L}) polypeptide that comprises CDR 1, 2 and 3 sequences of the V_{L} chain of the same other antibody having binding specificity (and preferably a strain agnostic binding specificity) to the first epitope within a polypeptide consisting of the amino acid sequence of SEQ ID NO: 177 in ECD1 (and optionally wherein the binding molecule also has binding specificity to a second epitope within ECD3) of US28 (and/or a functional variant of any one, two or three of said CDP sequences). Said other antibody can, for example, be any of antibodies 4A11A11, 2G2B3, 5D6H11, 9G3B9, 5E8F7, 6G3D6, 5E1E4, 13-5C6-1B5 and 14-1H3-1A6 as described herein, and/or another antibody that is obtained by the method of obtaining further antibodies having binding specificity to an epitope within SEQ ID NO: 177 of ECD1 and/or ECD3 of a US28 protein of HCMV, as described above.

When the antibody is defined as having a light chain variable region comprising a particular amino acid sequence and a heavy chain variable region having a particular amino acid sequence, it will be appreciated that up to one or two of those sequences may be varied as defined. The variation may be solely within the non-CDR regions of the sequences, solely within the CDR regions of the sequences, or within both the non-CDR and CDR regions of the sequences. Typically, the variation is outside of the CDP regions, and so the variants of the light chain variable regions and heavy chain variable regions, may comprise any of the particular CDR sequences defined herein.

Where the antibody comprises a variant of a heavy chain variable region and/or a light chain variable region as defined herein (e.g. a V_{H} selected from SEQ ID NOs: 187, 211, 233, 12, 104, 122, 130, 68 and 88 and/or a V_{L} selected from SEQ ID NOs: 189, 213, 235, 18, 108, 126, 134, 72 and 92), the variant may have at least 1, 2, 3, 4, or 5 amino acid substitutions. Typically, the variants do not have more than 30, 20, or 10 amino acid substitutions. Hence, the variants may have at least 1, 2, 3, 4 or 5 amino acid substitutions but not more than 30, 20 or 10 amino acid substitutions.

Where the antibody comprises a variant of a heavy chain variable region and/or a light chain variable region as defined herein (e.g. a V_{H} selected from SEQ ID NO: 187, 211, 233, 1.2, 1.04, 122, 130, 68 and 88 and/or a V_{L} selected from SEQ ID NO: 189, 213, 235, 18, 108, 126, 134, 72 and 92), the variant generally has at least 70% sequence identity to the defined amino acid sequence, for example at least 75%, 80%, 85%, 90% or 95% sequence identity, and more generally has 95-99% sequence identity to the defined amino acid sequence (e.g. 96%, 97%, 98% or 99% sequence identity). The level of variation may be applicable solely to the non-CDR region. For example, a variant of a heavy chain variable region and/or a light chain variable region as defined herein (e.g. a V_{H} selected from SEQ ID NO: 187, 211, 233, 12, 104, 122, 130, 68 and 88 and/or a V_{L} selected from SEQ ID NO: 189, 213, 235, 18, 108, 126, 134, 72 and 92), may have at least 70% identity (e.g. 75%, 80%, 85%, 90%, 95%, 96%, 97%, 98% or 99% sequence identity) to the defined amino acid sequence but comprise one or more (such as all) identical CDRs as defined herein.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally. The alignment may alternatively be carried out using the Clustal W program (Thompson et al., (1994) Nucleic Acids Res 22, 4673-80). The parameters used may be as follows: Fast pairwise alignment parameters: K-tuple(word) size; 1, window size; 5, gap penalty; 3, number of top diagonals; 5. Scoring method: x percent. Multiple alignment parameters: gap open penalty; 10, gap extension penalty; 0.05. Scoring matrix: BLOSUM.

Binding molecule of the first aspect of the present invention that comprise functional variants of the variable heavy chain (V_{H}) and/or variable light chain (V_{L}) polypeptide sequence of antibody 4H3C3, 7B1F3 and/or 2F5B11 as defined by SEQ ID NOs: 187, 211 and 233 for the V_{H} and 189, 213 and 235 for the V_{L}, respectively, or a 4H3C3 or 7B1F3 humanised variant thereof, preferably possess one or more binding properties that are similar or substantially equivalent to the binding properties of 4H3C3, 7B1F3 and/or 2F5B11, when tested under the same conditions as 4H3C3, 7B1F3 and/or 2F5B11, respectively, as further described above. Optionally, in a multispecific format, the binding molecule of the first aspect of the present invention that further comprise functional variants of the variable heavy chain (V_{H}) and/or variable light chain (V_{L}) polypeptide sequence of antibody 1D3, 1C10, 1A10, 1G9 and/or 1E8 as defined by SEQ ID NOs: 12, 104, 122, 68 and 88 for the V_{H} and 18, 108, 126, 72 and 92 for the V_{L}, respectively, preferably possess one or more binding properties that are similar or substantially equivalent to the binding properties of 1D3, 1C10, 1A10, 1G9 and/or IE8, when tested under the same conditions as 1D3, 1C10, 1A10, 1G9 and/or 1ES, respectively, as further described above.

Typically, it is preferred that the amino acid substitutions of the variants disclosed herein are conservative amino acid substitutions, for example where an amino acid residue is replaced with an amino acid residue having a similar side chain. Conservative amino acid substitutions are well known in the art and include (original residue→substitution) Ala (A)→Val, Gly or Pro; Arg (R)→Lys or His; Asn (N)→Gin; Asp (D)→Glu; Cys (C)→Ser; Gin (Q)→Asn; Glu (G)→Asp; Gly (G)→Ala; His (H)→Arg; Ile (I)→Leu; Leu (L)→Ile, Val or Met; Lys (K)→Arg; Met (M)→Leu; Phe (F)→Tyr; Pro (P)→Ala; Ser (S)→Thr or Cys; Thr (T)→Ser; Trp (W)→Tyr; Tyr (Y)→Phe or Trp; and Val (V)→Leu or Ala.

### Antibodies:

In one non-limiting, but preferred embodiment, a binding molecule according to the first aspect of the present invention may be, of comprise, an antibody.

The term “antibody” as used herein optionally includes antigen-binding fragments of said antibody. The term “chimeric antigen receptor (CAR)” as used herein optionally includes antigen-binding fragments of said CAR.

By “antibody or antigen-binding fragment thereof” we include substantially intact antibody molecules, as well as chimeric antibodies, humanised antibodies, isolated human antibodies, single chain antibodies, monospecific antibodies, bispecific antibodies, antibody heavy chains, antibody light chains, homodimers and heterodimers of antibody heavy and/or light chains, and antigen-binding fragments and derivatives of the same. Suitable antigen-binding fragments and derivatives include Fv fragments (e.g. single chain Fv and disulphide-bonded Fv), Fab-like fragments (e.g. Fab fragments, Fab′ fragments and F(ab)2 fragments), single variable domains (e.g. V_{H} and V_{L} domains) and single domain antibodies (dAbs, including single and dual formats [i.e. dAb-linker-dAb], and nanobodies). The potential advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from recombinant sources (e.g. CHO cells, E coli, etc.) thus allowing the facile production of large amounts of the said fragments.

By “an antibody” or “an antigen-binding fragment thereof” we include substantially intact antibody molecules, as well as chimeric antibodies, humanised antibodies, and isolated human antibodies.

An “an antibody”, including an “antigen-binding fragment thereof”, a CAR or antigen-binding fragment thereof, and/or other binding molecule in accordance with the first aspect of the present invention may, for example:
i. have a valency of “n”, wherein the n is an integer of one or more, and so, for example, may be monovalent, bivalent, trivalent or multivalent; and/or
ii. have binding specificity to one antigen, or to two or more different antigens, for example it may have the ability to bind specifically to “x” different antigens, wherein x is an integer of one, or two or more, subject to the requirement that at least one antigen is the ECD3 of the US28 protein; for example, it may have mono-specific, bi-specific, tri-specific or multi-specific binding specificity.

Exemplary forms of antibody or antigen-binding fragments thereof can include any one or more of single chain antibodies, bispecific antibodies, antibody heavy chains, antibody light chains, homodimers and heterodimers of antibody heavy and/or light chains, and antigen-binding fragments and derivatives of the same. Suitable antigen-binding fragments and derivatives include Fv fragments (e.g. single chain Fv and disulphide-bonded Fv), Fab-like fragments (e.g. Fab fragments, Fab′ fragments and F(ab)2 fragments), single variable domains (e.g. V_{H} and V_{L} domains) and single domain antibodies (dAbs, including single and dual formats [i.e. dAb-linker-dAb], and nanobodies). The potential advantages of using antibody fragments, rather than whole antibodies, are several-fold. The smaller size of the fragments may lead to improved pharmacological properties, such as better penetration of solid tissue. Moreover, antigen-binding fragments such as Fab, Fv, single chain Fv (ScFv) and dAb antibody fragments can be expressed in and secreted from recombinant host cells, such as E. coli, thus allowing the facile production of large amounts of the said fragments.

The term “bi-specific” as used herein means the polypeptide is capable of specifically binding at least two target entities. Each of these target entities may be to epitopes derived from the same protein (e.g. binding to a first epitope and second epitope of the same protein, e.g. US28). Alternatively, the target entities may be to epitopes derived from different proteins (e.g. binding to a first epitope of a first protein, and a second epitope of a second, different target, such as a different protein).

By “ScFv molecules” we mean molecules wherein the V_{H} and V_{L} partner domains are linked via a flexible oligopeptide. Engineered antibodies, such as ScFv antibodies, can be made using the techniques and approaches long known in the art. The advantages of using antibody fragments, rather than whole antibodies, are several-fold. The smaller size of the fragments may lead to improved pharmacological properties, such as better penetration to the target site. Effector functions of whole antibodies, such as complement binding, are removed. Fab, Fv, ScFv and dAb antibody fragments can all be expressed in and secreted from E. coli, thus allowing the facile production of large amounts of the fragments. Whole antibodies, and F(ab)₂ fragments are “bivalent”. By “bivalent” we mean that the antibodies and F(ab′)2 fragments have two antigen combining sites. In contrast, Fab, Fv, ScFv and dAb fragments are monovalent, having only one antigen combining site.

The phrases “an antibody” or “an antigen-binding fragment thereof” is also intended to encompass antibody mimics (for example, non-antibody scaffold structures that have a high degree of stability yet allow variability to be introduced at certain positions). Those skilled in the art of biochemistry will be familiar with many such molecules, as discussed in Gebauer & Skerra, 2009. Exemplary antibody mimics include: affibodies (also called Trinectins; Nygren, 2008, FEBS J, 275, 2668-2676); CTLDs (also called Tetranectins; Innovations Pharmac. Technol. (2006), 27-30); adnectins (also called monobodies; Meth. Mol. Biol., 352 (2007), 95-109); anticalins (Drug Discovery Today (2005), 10, 23-33); DARPins (ankyrins; Nat. Biotechnol. (2004), 22, 575-582); avimers (Nat. Biotechnol. (2005), 23, 1556-1561); microbodies (FEBS J, (2007), 274, 86-95); peptide aptamers (Expert. Opin. Biol. Ther. (2005), 5, 783-797); Kunitz domains (J. Pharmacol. Exp. Ther. (2006) 318, 803-809); affilins (Trends. Biotechnol. (2005), 23, 514-522); affimers (Avacta Life Sciences, Wetherby, UK).

Persons skilled in the art will further appreciate that the invention also encompasses modified versions of antibodies and antigen-binding fragments thereof, whether existing now or in the future, e.g. modified by the covalent attachment of polyethylene glycol or another suitable polymer (see below) and/or covalently or non-covalently attached (e.g. by presentation as a fusion protein) to other polypeptide sequences.

Methods of generating antibodies and antibody fragments are well known in the art. For example, antibodies may be generated via any one of several methods which employ induction of in vivo production of antibody molecules, screening of immunoglobulin libraries (Orlandi et al., 1989; Winter et al., 1991) or generation of monoclonal antibody molecules by cell lines in culture. These include, but are not limited to, the hybridoma technique, the human B-cell hybridoma technique, and the Epstein-Barr virus (EBV)-hybridoma technique (Kohler at al., 1975. Nature 256:4 950497; Kozbor et al., 1985. J. Immund. Methods 81:31-42; Cote et al., 1983. Proc. Natl. Acad. Sci. USA 80:202 6-2030; Cole et al., 1984. Mol. Cell. Biol. 62:109-120)

Suitable methods for the production of monoclonal antibodies are also disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982).

The term “monoclonal antibody” as used herein includes reference to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesised by a hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al, Nature, 256: 495 (1975), or may be made by recombinant DNA methods (see, e.g., U.S. Pat. No. 4,816,567). The “monoclonal antibodies” may also be isolated from phage antibody libraries using the techniques described in Clackson et al, Nature, 352:624-628 (1991) and Marks et al, J. Mol. Biol., 222:581-597 (1991), for example. Optionally, a substantially homogeneous population of monoclonal antibodies is a population in which greater than 80%, 85%, 90%, 95%, 96%, 97%, 98%, 99%, 99.5% or greater than 99.5% (such as 100%) of the antibodies in the population, by weight, are identical or substantially identical. Substantially identical antibodies bind to an identical epitope and/or comprise identical CDR sequences, identical V_{H} and/or V_{L} chains, identical heavy and/or light chains, or any combination thereof. Optionally monoclonal antibodies within a population can be considered identical irrespective of any potential enzymatic and/or nonenzymatic heterogeneity which may occur within such populations, for example, due to common modifications such as incomplete disulfide bond formation, glycosylation, N-terminal pyroglutamine cyclization, C-terminal lysine processing, deamidation, isomerization, and oxidation, and less common ones such as modification of the N-terminal amino acids by maleuric acid and amidation of the C-terminal amino acid. Such modifications and other such forms of heterogenity are discussed, for example, in Liu et al, J Pharm Sci. 2008 July; 97(7):2426-47, the contents of which are incorporated herein by reference.

Likewise, antibody fragments can be obtained using methods well known in the art (see, for example, Harlow & Lane, 1988, “Antibodies: A Laboratory Manual”, Cold Spring Harbor Laboratory, New York). For example, antibody fragments according to the present invention can be prepared by proteolytic hydrolysis of the antibody or by expression in E. coli or mammalian cells (e.g. Chinese hamster ovary [CHO] cell culture or other protein expression systems) of DNA encoding the fragment. Alternatively, antibody fragments can be obtained by pepsin or papain digestion of whole antibodies by conventional methods.

It will be appreciated by persons skilled in the art that for human therapy and/or diagnostics, human or humanised antibodies are preferably used. Humanised forms of non-human (e.g. murine) antibodies are genetically engineered chimeric antibodies or antibody fragments (e.g. antigen-binding fragments) having preferably minimal portions derived from non-human antibodies. Humanised antibodies include antibodies in which complementary determining regions (CDRs) of a human antibody (recipient antibody) are replaced by residues from a complementary determining region of interest, from a non-human species (donor antibody) such as mouse, rat or rabbit having the desired functionality. In some instances, Fv framework residues of the human antibody are replaced by corresponding non-human residues. Humanised antibodies may also comprise residues which are found neither in the recipient antibody nor in the imported complementarity determining region or framework sequences. In general, the humanised antibody will comprise substantially all of at least one, and typically two, variable domains, in which all or substantially all of the complementarity determining regions correspond to those of a donor antibody of interest, such as a non-human antibody and all, or substantially all, of the framework regions correspond to those of a relevant human consensus sequence. Humanised antibodies optimally also include at least a portion of an antibody constant region, such as an Fc region, typically derived from a human antibody (see, for example, Jones et al., 1986. Nature 321:522-525; Riechmann et al., 1988, Nature 332:323-329; Presta, 1992, Curr. Op. Struct. Biol. 2:593-596, which are incorporated herein by reference).

Methods for humanising non-human antibodies are well known in the art. Generally, the humanised antibody has one or more amino acid residues introduced into it from a source which is non-human. These non-human amino acid residues, often referred to as imported residues, are typically taken from an imported variable domain. Humanisation can be essentially performed as described (see, for example, Jones et al., 1986; Reichmann et al., 1988; Verhoeyen et al., 1988, Science 239:1534-1536I; U.S. Pat. No. 4,816,567, which are incorporated herein by reference) by substituting human complementarity determining regions with corresponding rodent complementarity determining regions. Accordingly, such humanised antibodies are chimeric antibodies, wherein substantially less than an intact human variable domain has been substituted by the corresponding sequence from a non-human species. In practice, humanised antibodies may be typically human antibodies in which some complementarity determining region residues and possibly some framework residues are substituted by residues from analogous sites in rodent antibodies.

Human antibodies can also be identified using various techniques known in the art, including phage display libraries (see, for example, Hoogenboom & Winter, 1991, J. Mol. Biol. 227:381; Marks et al., 1991, J. Mol. Biol. 222:581; Cole et al., 1985, In: Monoclonal antibodies and Cancer Therapy, Alan R. Liss, pp. 77; Boerner et al., 1991. J. Immunol. 147:86-95, Soderlind et al., 2000, Nat Biotechnol 18:852-6 and WO 98/32845, which are incorporated herein by reference).

It will be appreciated by persons skilled in the art that the polypeptides, e.g. antibodies, of the present invention may be of any suitable structural format.

Antibodies may also be comprised of an Fc region, for which there are Fc-specific receptors. Engineering the Fc region of a therapeutic monoclonal antibody or Fc fusion protein allows the generation of molecules that are better suited to the pharmacology activity required of them (Strohl, 2009, Curr Opin Biotechnol 20(6):685-91, the disclosures of which are incorporated herein by reference).

### 5. (a) Engineered Fc Regions for Increased Half-Life

One approach to improve the efficacy of a therapeutic antibody is to increase its serum persistence, thereby allowing higher circulating levels, less frequent administration and reduced doses.

The half-life of an IgG depends on its pH-dependent binding to the neonatal receptor FcRn. FcRn, which is expressed on the surface of endothelial cells, binds the IgG in a pH-dependent manner and protects it from degradation.

Some antibodies that selectively bind the FcRn at pH 6.0, but not pH 7.4, exhibit a higher half-life in a variety of animal models.

Several mutations located at the interface between the CH2 and CH3 domains, such as T250Q/M428L (Hinton et al., 2004, J Biol Chem. 279(8):6213-6, the disclosures of which are incorporated herein by reference) and M252Y/S254T/T256E+H433K/N434F (Vaccaro et al., 2005, Nat Biotechnol. 23(10):1283-8, the disclosures of which are incorporated herein by reference), have been shown to increase the binding affinity to FcRn and the half-life of IgG1 in vivo.

### 6. (b) Engineered Fc Regions for Altered Effector Function

The affinity of the Fc portion of an antibody to FcγRI, FcγRII and III can be compared with wild-type IgG1 by flow cytometry, to determine by what concentration half maximal binding is achieved of FcγR expressing cells (Hezareh et al., 2001, J Virol., 75(24): 12161-12168, incorporated herein by reference). This can alternatively be determined by FcγR enzyme linked immunosorbent assays (ELISA) (Shields et al., 2001, Mol. Basis Cell Dev. Biol., 276(9): 6591-6604, incorporated herein by reference).

The four human IgG isotypes bind the activating Fcγ receptors (FcγRI, FcγRIIa, FcγRIIIa), the inhibitory FcγRIIb receptor, and the first component of complement (C1q) with different affinities, yielding very different effector functions (Bruhns et al., 2009, Blood. 113(16):3716-25, the disclosures of which are incorporated herein by reference). IgG1 molecules have the highest affinity and capacity to induce effector functions, whereas IgG2, IgG3 and IgG4 are less effective (Bruhns, 2012, Blood. 119(24):5640-9; Hogarth and Pietersz, 2012, Nature Reviews Drug Discovery 11, 311-331; Stewart et al. 2014 Journal for ImmunoTherapy of Cancer 2:29; Wang et al. 2015 Front Immunol; 6: 368; Vidarsson et al. 2014, Front Immunol. 5:520, incorporated herein by reference). In addition, certain mutations in the Fc region of IgG1 dramatically reduce FcγR affinity and effector function while retaining neonatal FcR (FcRn) interaction (Ju and Jung, 2014, Curr Opin Biotechnol. 30: 128-39; Leabman et al. 2013, mAbs, 5:6, 896-903; Oganesyan et al. 2008 Acta Crystallogr D Biol Crystallogr. 64 (Pt 6): 700-704; Oganesyan et al. 2008 Mol Immunol. 45(7):1872-82; Sazinsky et al., 2008 Proc. Natl. Acad. Sci. U.S.A 105(51) 20167-20172, the disclosures of which are incorporated herein by reference).

The most widely used IgG1 mutants are N297A alone or in combination with D265A, as well as mutations at positions L234 and L235, including the so-called “LALA” double mutant L234A/L235A. Another position described to further silence IgG1 by mutation is P329 (see US 2012/0251531). Additional mutations in the Fc region are described in WO 2021/234402. Accordingly, binding molecules of the present invention may incorporate any of the Fc regions as described in WCO 2021/234402, the contents of which are incorporated herein by reference.

In exemplary embodiments, the polypeptide is selected from the groups consisting of (any of which may be monospecific or bispecific):
(a) bivalent antibodies, such as IgG-scFv antibodies (for example, wherein a first binding domain is an intact IgG and a second binding domain is an scFv attached to first binding domain at the N-terminus of a light chain and/or at the C-terminus of a light chain and/or at the N-terminus of a heavy chain and/or at the C-terminus of a heavy chain of the IgG, or vice versa);
(b) monovalent antibodies, such as a DuoBody® (Genmab AS, Copenhagen, Denmark) or ‘knob-in-hole’ bispecific antibody (for example, an scFv-KIH, scFv-KIH^{r}, a BiTE-KIH or a BiTE-KIH^{r} (see Xu et al., 2015, mAbs 7(1):231-242);
(c) scFv₂-Fc antibodies (such as ADAPTIR™ bispecific antibodies from Emergent Biosolutions Inc);
(d) BiTE/scFv₂ antibodies;
(e) DVD-Ig antibodies;
(f) DART-based antibodies (for example, DART₂-Fc or DART);
(g) DNL-Fab₃ antibodies; and
(h) scFv-HSA-scFv antibodies.

For example, the antibody may be an IgG-scFv antibody. The IgG-scFv antibody may be in either VH-VL or VL-VH orientation. In one embodiment, the scFv may be stabilised by a S—S bridge between VH and VL.

A first binding domain and second binding domain may be fused directly to each other. Alternatively, a first binding domain and second binding domain may be joined via a linker, such as a polypeptide linker. For example, a polypeptide linker may be a short linker peptide between about 10 to about 25 amino acids. The linker is usually rich in glycine for flexibility, as well as serine or threonine for solubility, and can either connect the N-terminus of the V_{H} with the C-terminus of the V_{L}, or vice versa.

The terms “binding activity” and “binding affinity” are intended to refer to the tendency of a polypeptide molecule to bind or not to bind to a target. Binding affinity may be quantified by determining the dissociation constant (Kd) for a polypeptide and its target. A lower Kd is indicative of a higher affinity for a target. Similarly, the specificity of binding of a polypeptide to its target may be defined in terms of the comparative dissociation constants (Kd) of the polypeptide for its target as compared to the dissociation constant with respect to the polypeptide and another, non-target molecule.

The value of this dissociation constant can be determined directly by well-known methods, and can be computed even for complex mixtures by methods such as those, for example, set forth in Caceci et al., 1984. For example, the Kd may be established using a double-filter nitrocellulose filter binding assay such as that disclosed by Wong & Lohman, 1993. Other standard assays to evaluate the binding ability of ligands such as antibodies towards targets are known in the art, including for example, ELISAs, Western blots, RIAs, and flow cytometry analysis. The binding kinetics (e.g. binding affinity) of the polypeptide also can be assessed by standard assays known in the art, such as by Biacore™ system analysis.

A competitive binding assay can be conducted in which the binding of the polypeptide to the target is compared to the binding of the target by another, known ligand of that target, such as another polypeptide. The concentration at which 50% inhibition occurs is known as the Ki. Under ideal conditions, the Ki is equivalent to Kd. The Ki value will never be less than the Kd, so measurement of Ki can conveniently be substituted to provide an upper limit for Kd.

Alternative measures of binding affinity include EC50 or IC50. In this context EC50 indicates the concentration at which a polypeptide achieves 50% of its maximum binding to a fixed quantity of target. IC50 indicates the concentration at which a polypeptide inhibits 50% of the maximum binding of a fixed quantity of competitor to a fixed quantity of target. In both cases, a lower level of EC50 or IC50 indicates a higher affinity for a target. The EC50 and IC50 values of a ligand for its target can both be determined by well-known methods, for example ELISA. Suitable assays to assess the EC50 and IC50 of polypeptides are set out in the Examples.

As mentioned, antibodies may be produced by standard techniques, for example by immunisation with the appropriate (glyco)polypeptide or portion(s) thereof, or by using a phage display library.

If polyclonal antibodies are desired, a selected mammal (e.g. mouse, rabbit, goat, horse, etc) is immunised with an immunogenic polypeptide bearing a desired epitope(s), optionally haptenised to another polypeptide. Depending on the host species, various adjuvants may be used to increase immunological response. Such adjuvants include, but are not limited to, Freund's, mineral gels such as aluminium hydroxide, and surface-active substances such as lysolecithin, pluronic polyols, polyanions, peptides, oil emulsions, keyhole limpet hemocyanin, and dinitrophenol. Serum from the immunised animal is collected and treated according to known procedures. If serum containing polyclonal antibodies to the desired epitope contains antibodies to other antigens, the polyclonal antibodies can be purified by imnunoaffinity chromatography. Techniques for producing and processing polyclonal antisera are well known in the art.

Monoclonal antibodies directed against entire polypeptides or particular epitopes thereof (for example, in the case of the present application, against a first epitope within a polypeptide consisting of the amino acid sequence of TDVLNQSKPVTL (SEQ ID NO: 177) within the ECD1, and/or optionally ECD3, of US28, can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against the polypeptides listed above can be screened for various properties; i.e. for isotype and epitope specificity and/or affinity, as well as strain agnostic binding properties. Monoclonal antibodies may be prepared using any of the well-known techniques which provides for the production of antibody molecules by continuous cell lines in culture.

In some embodiments, it may be preferred if the antibody is a monoclonal antibody. In some circumstance, particularly if the antibody is to be administered repeatedly to a human patient, it is preferred if the monoclonal antibody is a human monoclonal antibody or a humanised monoclonal antibody, which are suitable for administration to humans without engendering an immune response by the human against the administered immunoglobulin. Suitably prepared non-human antibodies can be “humanised” in known ways, for example by inserting the CDR regions of mouse antibodies into the framework of human antibodies. Humanised antibodies can be made using the techniques and approaches described in Verhoeyen et al., (1988) Science, 239, 1534-1536, and in Kettleborough et al., (1991) Protein Engineering, 14(7), 773-783. In some instances, Fv framework residues of the human immunoglobulin are replaced by corresponding non-human residues. In general, the humanised antibody will contain variable domains in which all or most of the CDR regions correspond to those of a non-human immunoglobulin, and framework regions which are substantially or completely those of a human immunoglobulin consensus sequence.

Completely human antibodies may be produced using recombinant technologies. Typically, large libraries comprising billions of different antibodies are used. In contrast to the previous technologies employing chimerisation or humanisation of, e.g., murine antibodies, this technology does not rely on immunisation of animals to generate the specific antibody. Instead the recombinant libraries comprise a huge number of pre-made antibody variants wherein it is likely that the library will have at least one antibody specific for any antigen. Thus, using such libraries, an existing antibody having the desired binding characteristics can be identified. In order to find the good binder in a library in an efficient manner, various systems where phenotype, i.e. the antibody or fragment thereof, is linked to its genotype, i.e. the encoding gene(s), have been devised. The most commonly used such system is the so-called phage display system where antibody fragments are expressed, displayed, as fusions with phage coat proteins on the surface of filamentous phage particles, while simultaneously carrying the genetic information encoding the displayed molecule (McCafferty et al., 1990, Nature 348: 552-554). Phage displaying antibody fragments specific for a particular antigen may be selected through binding to the antigen in question. Isolated phage may then be amplified and the gene encoding the selected antibody variable domains may optionally be transferred to other antibody formats, such as e.g. full-length immunoglobulin, and expressed in high amounts using appropriate vectors and host cells well known in the art. Alternatively, the “human” antibodies can be made by immunising transgenic mice which contain, in essence, human immunoglobulin genes (Vaughan et al., (1998) Nature Biotechnol. 16, 535-539).

It is appreciated that when the antibody is for administration to a non-human individual, the antibody may have been specifically designed/produced for the intended recipient species.

The format of displayed antibody specificities on phage particles may differ. The most commonly used formats are Fab (Griffiths et al., 1994. EMBO J. 13: 3245-3260) and scFv (Hoogenboom et al., 1.992, J Mol Biol. 227: 381-388) both comprising the variable antigen binding domains of antibodies. The single chain format is composed of a variable heavy domain (V_{H}) linked to a variable light domain (V_{L}) via a flexible linker (U.S. Pat. No. 4,946,778). Before use as a therapeutic agent, the antibody may be transferred to a soluble format e.g. Fab or scFv and analysed as such. In later steps the antibody fragment identified to have desirable characteristics may be transferred into yet other formats such as full-length antibodies.

WO 98/32845 and Soderlind et al., (2000) Nature BioTechnol. 18: 852-856 describe technology for the generation of variability in antibody libraries. Antibody fragments derived from this library all have the same framework regions and only differ in their CDRs. Since the framework regions are of germline sequence the immunogenicity of antibodies derived from the library, or similar libraries produced using the same technology, are expected to be particularly low (Soderlind et al., 2000, supra). This property is of great value for therapeutic antibodies, reducing the risk that the patient forms antibodies to the administered antibody, thereby reducing risks for allergic reactions, the occurrence of blocking antibodies, and allowing a long plasma half-life of the antibody. Thus, when developing therapeutic antibodies to be used in humans, modern recombinant library technology (Soderlind et al., 2001, Comb. Chem. & High Throughput Screen. 4: 409-416) is now used in preference to the earlier hybridoma technology.

By antibodies we also include heavy-chain antibodies structurally derived from camelidae antibodies, such as Nanobodies® (Ablynx). These are antibody-derived therapeutic proteins that contain the structural and functional properties of naturally-occurring heavy-chain antibodies. The Nanobody® technology was developed following the discovery that camelidae (camels and llamas) possess fully functional antibodies that lack light chains. These heavy-chain antibodies contain a single variable domain (VHH) and two constant domains (C_{H}2 and C_{H}3). The cloned and isolated VHH domain is a perfectly stable polypeptide harbouring the full antigen-binding capacity of the original heavy-chain antibody. These VHH domains with their unique structural and functional properties form the basis of Nanobodies® They combine the advantages of conventional antibodies (high target specificity, high target affinity and low inherent toxicity) with important features of small molecule drugs (the ability to inhibit enzymes and access receptor clefts). Furthermore, they are stable, have the potential to be administered by means other than injection, are easier to manufacture, and can be humanised. (See, for example U.S. Pat. Nos. 5,840,526; 5,874,541; 6,005,079, 6,765,087; EP 1 589 107; WO 97/34103; WO97/49805; U.S. Pat. Nos. 5,800,988; 5,874,541 and 6,015,695).

It is preferred that the antibody, or other binding molecule, that selectively binds to ECD3 of US28 does not bind a related polypeptide, such as CCR5, or that the antibody binds US28 with a greater affinity than for the related polypeptide, such as CCR5. Preferably, the antibody binds the US28 with at least 5, or at least 10 or at least 50 times greater affinity than for the related polypeptide. More preferably, the antibody molecule binds the US28 with at least 100, or at least 1,000, or at least 10,000 times greater affinity than for the related polypeptide. Such binding may be determined by methods well known in the art, such as one of the Biacore® systems.

### Bispecific Antibodies and Other Bispecific Binding Molecules

In one embodiment of particular interest, the binding molecule of the first aspect of the present invention is, or comprises, a bispecific binding molecule.

For example, a bispecific binding molecule in accordance with the first aspect of the present invention may comprise a first domain capable of recruiting the activity of an effector cell by specifically binding to an effector antigen located on the effector cell; and a second domain capable of specifically binding to a first epitope within a polypeptide consisting of the amino acid sequence of TDVLNQSKPVTL. (SEQ ID NO: 177) within the ECD1 of the HCMV-encoded US28 protein (and optionally a third domain capable of specifically binding to ECD3 of the HCMV-encoded US28 protein), as a target antigen, wherein said target antigen may be located on a target cell other than the effector cell. The second domain is, or comprises, a binding molecule according to the first aspect of the present invention. The third domain is, or comprises, a binding molecule according to the first aspect of the present invention that is distinct from the second domain.

All or part of the first, second and/or third domains may optionally be connected by one or more linker sequences. The first domain may comprise multiple functional domains and each of those functional domains may be connected to a neighbouring domain either directly or via a linker sequence and/or may be formed from more than one separate polypeptide sequence. The second domain may comprise multiple functional domains and each of those functional domains may be connected to a neighbouring domain either directly or via a linker sequence and/or may be formed from more than one separate polypeptide sequence. The third domain may comprise multiple functional domains and each of those functional domains may be connected to a neighbouring domain either directly or via a linker sequence and/or may be formed from more than one separate polypeptide sequence.

In the instances in which one or more linker sequence is present in a multispecific (e.g. bispecific or trispecific) binding molecule of the present invention, then each linker sequence may independently be selected from any suitable linker sequence. For example, each linker sequence may be at least 1, 2, 3, 4, 5, 6, 7, 8, 9, 10, 11, 12, 13, 14, 1.5, 16, 17, 1.8, 19, 20, 25, 30, 35, 40, 45, 50, or more amino acid residues in length. Each linker sequence may comprise any naturally occurring amino acid. In some embodiments, each linker sequence may comprise or consist of the amino acids glycine and serine. In another embodiment, the or each linker sequence may comprise or consist of sets of glycine and serine repeats such as (Gly₄Ser)ₙ, where n is a positive integer equal or greater than 1, such as 2, 3, 4, 5, 6 or more. In one embodiment, the linker is (Gly₄Ser)₃. Variation in the linker length may retain or enhance activity, giving rise to superior efficacy in activity studies.

In one embodiment, the bispecific binding molecule is a bispecific antibody (bsAb) format, such as those described in Suurs et al., 2019, Pharmacology & Therapeutics, 201: 103-119 (the content of which are incorporated herein by reference), and in particular one or more of the formats illustrated in FIG. 1C of Suurs et al., 2019 (supra), such as a TriMab, an IgG-like BsAb, a CrossMab, a 2:1 CrossMab, a 2:2 CrossMab, a DuoBody, a DVD-Ig BsAb, an scFv-IgG, and IgG-IgG, a Fab-scFv-Fc, a TF, an ADAPTIR, a BiTE, a BiTE-Fc, a DART, a DART-Fc, a Tetravalent DART, a TandAb, an ImmTAC, a TriKE, and scFv-scFv-scFv, or a tris-specific nanobody format.

In one embodiment, the first and/or second domain may comprise at least one variable light chain and/or at least one variable heavy chain. In one embodiment, the first and/or second domain may comprise at least one variable light chain and at least one variable heavy chain to form an scFv. In one embodiment, the first and/or second domain comprise a noncovalent dimer of scFv connected by a linker (e.g. a small peptide linker) to form a diabody. In one embodiment, the first and/or second domain may comprise multiple diabodies to form a tandem diabody (TandAb). Optionally, in some embodiments, any of the foregoing may further comprise a third domain, for example with binding specificity to ECD3 of US28.

In one embodiment, the first and/or second domain may further comprise at least one constant light chain and/or at least one constant heavy chain. For example, the first and/or second domain may comprise an scFv that further comprises a constant light chain and a constant heavy chain to form a Fab. In one embodiment, the first and/or second domain comprises multiple Fab (for example, two Fab). Optionally, in some embodiments, any of the foregoing may further comprise a third domain, for example with binding specificity to ECD3 of US28.

In one embodiment, the first and/or second domain may further comprise at least one Fc. For example, the first and/or second domain may comprise an scFv that further comprises an Fc to form an scFv-IgG. Additionally, or alternatively, the first and/or second domain may comprise two Fab domains that further comprises an Fc to form an IgG. Optionally, in some embodiments, any of the foregoing may further comprise a third domain, for example with binding specificity to ECD3 of US28.

In one embodiment, the first domain may be an scFv connected to a second domain that is an scFv, optionally via a linker, to form a BiTE, optionally wherein the BiTE further comprises an Fc to form a BiTE-Fc. Optionally, in some embodiments, any of the foregoing may further comprise a third domain, for example with binding specificity to ECD3 of US28, optionally via a linker.

In one embodiment, the V_{H} or V_{L} of the first domain may be swapped with the V_{H} or V_{L} of the second domain, respectively, to form a DART comprising a first and second domain, optionally wherein the DART further comprises an Fc to form a DART-Fc. Additionally, or alternatively, multiple DARTs may be connected to a single Fc to form a tetravalent DART.

In one embodiment, the first and/or second domain is in the form of a T cell receptor, which may be combined with a first and/or second domain that is in the form of an scFv to form an ImmTAC. For example, the first domain may be an scFv and the second domain may be a T cell receptor, preferably wherein the scFv is specific for an epitope of CD3 (i.e. a CD3-binding domain), thereby resulting in an ImmTAC. Optionally, in some embodiments, any of the foregoing may further comprise a third domain, for example with binding specificity to ECD3 of US28.

In one embodiment, the effector cell of the first domain is an immune effector cell, for example an immune effector cell selected from the group consisting of a T cell (for example, a CD4⁺ T cell and/or a CD8⁺ T cell), NK T cell, NK cell, macrophage, or any recombinant cell thereof (for example, a CAR-expressing cells, such as a CAR-T cell, a CAR-NK cell or a CAR-M cell). In such embodiments, the bispecific binding molecule may be referred to as a ‘bispecific immune cell engager antibody’, wherein the “immune cell” may be substituted for the effector cell type. For example, if the effector cell of the first domain is a T cell, such embodiments may be referred to as a ‘bispecific T cell engager antibody’ (BiTE, which may, but does not necessarily have the format of the BiTE or BiTE-Fc molecule shown in FIG. 1C of Suurs et al, 2019, supra), and if the effector cell of the first domain is an NK cell, such embodiments may be referred to as a ‘bispecific NK cell engager antibody’ and so on.

In one embodiment, the first domain of the bispecific binding molecule is specific for an epitope of CD3, which may be referred to as a CD3-binding domain.

In one embodiment, the CD3-binding domain may comprise the sequence of OKT3 heavy chain variable region, for example as defined by the sequence of SEQ ID NO: 48 of the present application, or a functional variant or fragment thereof which substantially retains the CD3-binding specificity and/or affinity of the sequence of SEQ ID NO: 48. The functional variant or fragment thereof may optionally comprises one, two or three CDRs corresponding to any one, two or all three of the CDR sequences of the OKT3 heavy chain variable region as defined by SEQ ID NO: 48. The CDR sequences of OKT3 heavy chain variable region are as defined by SEQ ID NOs: 49, 50 and 51, respectively, and/or a functional variant of any one or more of said CDR sequences of antibody OKT3.

In another embodiment, the CD3-binding domain may comprise the sequence of OKT3 light chain variable region, for example as defined by the sequence of SEQ ID NO: 52 of the present application, or a functional variant or fragment thereof which substantially retains the CD3-binding specificity and/or affinity of the sequence of SEQ ID NO: 52. The functional variant or fragment thereof may optionally comprises one, two or three CDRs corresponding to any one, two or all three of the CDR sequences of the OKT3 light chain variable region as defined by SEQ ID NO: 52. The CDR sequences of OKT3 heavy chain variable region are as defined by SEQ ID NOs: 53, 54 and 55, respectively, and/or a functional variant of any one or more of said CDR sequences of antibody OKT3.

In another embodiment, the CD3-binding domain may comprise both of the sequences of OKT3 heavy chain variable region and the OKT3 light chain variable region, for example as defined by the sequences of SEQ ID NOs: 48 and 52 of the present application, respectively, or a functional variant or fragment thereof of either or both, such as a functional variant or fragment as described above.

In a preferred embodiment, the CD3-binding domain may comprise both of the sequences of OKT3 heavy chain variable region and the OKT3 light chain variable region, for example as defined by the sequences of SEQ ID NOs: 48 and 52 of the present application, optionally joined by a linker, such as a linker having the sequence GGGGSGGGGSGGGGS (SEQ ID NO: 56). This combination of an OKT3 heavy chain variable region and an OKT3 light chain variable region, optionally joined by a linker, may form an scFv of OKT3.

Accordingly, the CD3-binding domain may be an scFv of OKT3 that comprises the sequences of SEQ ID Nos: 48, 56 and 52, joined together in that order, wherein the OKT3 heavy chain variable region of SEQ ID NO: 48 is joined at its C-terminus to the N-terminus of the linker sequence of SEQ ID NO: 56, which in turn is joined at its C-terminus to the N-terminus of the OKT3 light chain variable region of SEQ ID NO: 52. Alternatively, the order of the light and heavy chain regions may be swapped around, so that the CD3-binding domain comprises the sequences of SEQ ID Nos: 52, 56 and 48, joined together in that order. It will be appreciated that other linker sequences may be used in place of the exemplary sequence of SEQ ID NO: 56; other linker sequences may optionally comprise multiple repeats of the sequence GGGGS (SEQ ID NO: 57), and thus may have the sequence [GGGGS]ₙ, wherein n is an integer of 2, 3, 4, 5, 6, 7, 8, 9 10 or more; or may contain any other suitable linker sequences.

Optionally, the CD3-binding domain comprises at least one (preferably two) scFvs of OKT3. For example, the bispecific binding molecule may comprise a first domain comprising two scFvs of OKT3, optionally wherein the two scFv are linked directly to each other or linked indirectly via the second domain.

Optionally, the binding molecules of the invention, when put into an OKT3 bispecific format, can reduce tumour size relative to a control (for example, relative to a monospecific antibody that exclude an OKB3-binding domain). Such a reduction in tumour size can be assessed, for example, using the techniques described in Example 10.

In one embodiment, the second domain of the bispecific binding molecule, which comprises a binding molecule according to the first aspect of the present invention and has binding specificity to a first epitope within a polypeptide consisting of the amino acid sequence of TDVLNQSKPVTL (SEQ ID NO: 177) within ECD1 of the US28 protein of HCMV, has binding specificity for a target cell that is a US28-expressing cell and/or a HCMV infected cell (which may, for example, be latently infected or lytically infected).

In one embodiment, the second domain of the bispecific binding molecule comprises one, two, three, four, five or six complementarity determining regions (CDRs) having binding specificity to a first epitope within a polypeptide consisting of the amino acid sequence of TDVLNQSKPVTL (SEQ ID NO: 177), said CDRs corresponding to any one, two, three, four, five or all six of the CDR sequences of antibody:
(a) 4H3C3 as defined by SEQ ID NOs: 196, 197, 198, 199, 200 and 201, respectively;
(b) 7B1F3 as defined by SEQ ID NOs: 219, 220, 221, 199, 222 and 223, respectively; and/or
(c) 2F5B11 as defined by SEQ ID NOs: 242, 243, 244, 199, 245 and 246, respectively;
and/or a functional variant of any one or more of said CDR sequences of antibody 4H3C3, 7B1F3 and/or 2F5B11 (such as the 4H3C3 or 7B1F3 humanised variants described herein).

Optionally, the US28-binding domain according to this embodiment may comprise:
(a) one, two, or all three, of the CDR 1, 2, and 3, sequences of the variable heavy chain (V_{H}) of antibody:
   i. 4H3C3 as defined by SEQ ID NOs: 196; 197, 293 or 294; and 198, respectively (and/or a functional variant of any one, two or three of said CDR sequences);
   ii. 7B1F3 as defined by SEQ ID NOs: 219; 220, 296 or 297; and 221 or 298, respectively (and/or a functional variant of any one, two or three of said CDR sequences); and/or
   iii. 2F5B11 as defined by SEQ ID NOs: 242, 243, and 244, respectively (and/or a functional variant of any one, two or three of said CDR sequences);
and/or
(b) one, two, or all three, of the CDR 1, 2, and 3, sequences of the variable light chain (V_{L}) of antibody:
   i. 4H3C3 as defined by SEQ ID NOs: 199 or 295; 200 or 222; and 201, respectively (and/or a functional variant of any one, two or three of said CDR sequences);
   ii. 7B1F3 as defined by SEQ ID NOs: 199 or 295; 222, and 223, respectively (and/or a functional variant of any one, two or three of said CDR sequences); and/or
   iii. 2F5B11 as defined by SEQ ID NOs: 199, 245, and 246, respectively (and/or a functional variant of any one, two or three of said CDR sequences).

Preferably, the US28-binding domain according to this embodiment comprises at least 4 different CDR sequences, for example at least 5 or 6 different CDR sequences.

Additionally, or alternatively, in a further option the second domain of the bispecific binding molecule according to this embodiment may comprise: (a) at least one variable heavy chain (V_{H}) polypeptide that comprises CDR 1, 2, and 3 sequences having the sequences of SEQ ID NOs: 196, 197 and 198 (or humanised variants thereof), respectively, and optionally wherein the at least one variable heavy chain (V_{H}) polypeptide comprises, consists essentially of, or consists of, the sequence of SEQ ID NO: 187 (or a humanised variant thereof); and/or (b) at least one variable light chain (V_{L}) polypeptide that comprises CDR 1, 2 and 3 sequences having the sequences of SEQ ID NOs: 199, 200 and 201 (or humanised variants thereof), respectively, and optionally wherein the variable light chain (V_{L}) polypeptide comprises, consists essentially of, or consists of, the sequence of SEQ ID NO: 189 (or a humanised variant thereof).

Additionally, or alternatively, in a further option the second domain of the bispecific binding molecule according to this embodiment may comprise: (a) at least one variable heavy chain (V_{H}) polypeptide that comprises CDR 1, 2, and 3 sequences having the sequences of SEQ ID NOs: 219, 220 and 221 (or humanised variants thereof), respectively, and optionally wherein the at least one variable heavy chain (V_{H}) polypeptide comprises, consists essentially of, or consists of, the sequence of SEQ ID NO: 21.1 (or a humanised variant thereof); and/or (b) at least one variable light chain (V_{L}) polypeptide that comprises CDR 1, 2 and 3 sequences having the sequences of SEQ ID NOs: 199, 222 and 223 (or humanised variants thereof), respectively, and optionally wherein the variable light chain (V_{L}) polypeptide comprises, consists essentially of, or consists of, the sequence of SEQ ID NO: 213 (or a humanised variant thereof).

Additionally, or alternatively, in a further option the second domain of the bispecific binding molecule according to this embodiment may comprise: (a) at least one variable heavy chain (V_{H}) polypeptide that comprises CDR 1, 2, and 3 sequences having the sequences of SEQ ID NOs: 242, 243 and 244, respectively, and optionally wherein the at least one variable heavy chain (V_{H}) polypeptide comprises, consists essentially of, or consists of, the sequence of SEQ ID NO: 233; and/or (b) at least one variable light chain (V_{L}) polypeptide that comprises CDR 1, 2 and 3 sequences having the sequences of SEQ ID NOs: 199, 245 and 246, respectively, and optionally wherein the variable light chain (V_{L}) polypeptide comprises, consists essentially of, or consists of, the sequence of SEQ ID NO: 235.

In one embodiment, the third domain of the bispecific binding molecule, which comprises a binding molecule according to the first aspect of the present invention and has binding specificity to ECD3 of the US28 protein of HCMV, has binding specificity for a target cell that is a US28-expressing cell and/or a HCMV infected cell (which may, for example, be latently infected or lytically infected).

In one embodiment, the third domain of the bispecific binding molecule comprises one, two, three, four, five or six complementarity determining regions (CDRs) corresponding to any one, two, three, four, five or all six of the CDR sequences of antibody:
(a) 1D3 as defined by SEQ ID NOs: 8, 9, 10, 14, 15 and 16, respectively;
(b) 1C10 as defined by SEQ ID NOs: 112, 113, 114, 117, 83 and 118, respectively;
(c) 1A10 as defined by SEQ ID NOs: 112, 113, 114, 117, 83 and 118, respectively;
(d) 1G9 as defined by SEQ ID NOs: 76, 77, 78, 82, 83 and 84, respectively; and/or
(e) 1E8 as defined by SEQ ID NOs: 76, 95, 96, 82, 99 and 100, respectively;
and/or a functional variant of any one or more of said CDR sequences of antibody 1D3, 1C10, 1A10, 1G9 and/or 1E8.

Optionally, the US28-binding domain according to this embodiment may comprise:
(a) one, two, or all three, of the CDR 1, 2, and 3, sequences of the variable heavy chain (VW) of antibody:
   i. D3, as defined by SEQ ID NOs: 8, 9, and 10, respectively (and/or a functional variant of any one, two or three of said CDR sequences);
   ii. 1C10, as defined by SEQ ID NOs: 112, 113, and 114, respectively (and/or a functional variant of any one, two or three of said CDR sequences);
   iii. 1A10, as defined by SEQ ID NOs: 112, 113, and 114, respectively (and/or a functional variant of any one, two or three of said CDR sequences);
   iv. 1G9, as defined by SEQ ID NOs: 76, 77, and 78, respectively (and/or a functional variant of any one, two or three of said CDR sequences); and/or
   v. 1E8, as defined by SEQ ID NOs: 76, 95, and 96, respectively (and/or a functional variant of any one, two or three of said CDR sequences);
and/or
(b) one, two, or all three, of the CDR 1, 2, and 3, sequences of the variable light chain (V_{L}) of antibody:
   i. D3, as defined by SEQ ID NOs: 14, 15, and 16, respectively;
   ii. 1C10, as defined by SEQ ID NOs: 117, 83, and 118, respectively (and/or a functional variant of any one, two or three of said CDR sequences);
   iii. 1A10, as defined by SEQ ID NOs: 117, 83, and 118, respectively (and/or a functional variant of any one, two or three of said CDR sequences);
   iv. 1G9, as defined by SEQ ID NOs: 82, 83, and 84, respectively (and/or a functional variant of any one, two or three of said CDR sequences); and/or
   v. 1E8, as defined by SEQ ID NOs: 82, 99, and 100, respectively (and/or a functional variant of any one, two or three of said CDR sequences).

Preferably, the US28-binding domain according to this embodiment comprises at least 4 different CDR sequences, for example at least 5 or 6 different CDR sequences.

Additionally, or alternatively, in a further option the third domain of the bispecific binding molecule according to this embodiment may comprise: (a) at least one variable heavy chain (V_{H}) polypeptide that comprises CDR 1, 2, and 3 sequences having the sequences of SEQ ID NOs: 8, 9, and 10, respectively, and optionally wherein the at least one variable heavy chain (V_{H}) polypeptide comprises, consists essentially of, or consists of, the sequence of SEQ ID NO:12; and/or (b) at least one variable light chain (V_{L}) polypeptide that comprises CDR 1, 2 and 3 sequences having the sequences of SEQ ID NOs: 14, 15, and 16, respectively, and optionally wherein the variable light chain (V_{L}) polypeptide comprises, consists essentially of, or consists of, the sequence of SEQ ID NO:18.

Additionally, or alternatively, in a further option the third domain of the bispecific binding molecule according to this embodiment may comprise: (a) at least one variable heavy chain (V_{H}) polypeptide that comprises CDR 1, 2, and 3 sequences having the sequences of SEQ ID NOs: 76, 77, and 78, respectively, and optionally wherein the at least one variable heavy chain (V_{H}) polypeptide comprises, consists essentially of, or consists of, the sequence of SEQ ID NO: 68; and/or (b) at least one variable light chain (V_{L}) polypeptide that comprises CDR 1, 2 and 3 sequences having the sequences of SEQ ID NOs: 82, 83, and 84, respectively, and optionally wherein the variable light chain (V_{L}) polypeptide comprises, consists essentially of, or consists of, the sequence of SEQ ID NO: 72.

Additionally, or alternatively, in a further option the third domain of the bispecific binding molecule according to this embodiment may comprise: (a) at least one variable heavy chain (V_{H}) polypeptide that comprises CDR 1, 2, and 3 sequences having the sequences of SEQ ID NOs: 76, 95, and 96, respectively, and optionally wherein the at least one variable heavy chain (V_{H}) polypeptide comprises, consists essentially of, or consists of, the sequence of SEQ ID NO: 88; and/or (b) at least one variable light chain (V_{L}) polypeptide that comprises CDR 1, 2 and 3 sequences having the sequences of SEQ ID NOs: 82, 99, and 100, respectively, and optionally wherein the variable light chain (V_{L}) polypeptide comprises, consists essentially of, or consists of, the sequence of SEQ ID NO: 92.

Additionally, or alternatively, in a further option the third domain of the bispecific binding molecule according to this embodiment may comprise: (a) at least one variable heavy chain (V_{H}) polypeptide that comprises CDR 1, 2, and 3 sequences having the sequences of SEQ ID NOs: 112, 113, and 114, respectively, and optionally wherein the at least one variable heavy chain (V_{H}) polypeptide comprises, consists essentially of, or consists of, the sequence of SEQ ID NO: 104; and/or (b) at least one variable light chain (V_{L}) polypeptide that comprises CDR 1, 2 and 3 sequences having the sequences of SEQ ID NOs: 117, 83, and 118, respectively, and optionally wherein the variable light chain (V_{L}) polypeptide comprises, consists essentially of, or consists of, the sequence of SEQ ID NO: 108.

Additionally, or alternatively, in a further option the third domain of the bispecific binding molecule according to this embodiment may comprise: (a) at least one variable heavy chain (V_{H}) polypeptide that comprises CDR 1, 2, and 3 sequences having the sequences of SEQ ID NOs: 11.2, 113, and 114, respectively, and optionally wherein the at least one variable heavy chain (V_{H}) polypeptide comprises, consists essentially of, or consists of, the sequence of SEQ ID NO: 122; and/or (b) at least one variable light chain (V_{L}) polypeptide that comprises CDR 1, 2 and 3 sequences having the sequences of SEQ ID NOs: 117, 83, and 118, respectively, and optionally wherein the variable light chain (V_{L}) polypeptide comprises, consists essentially of, or consists of, the sequence of SEQ ID NO: 126.

Additionally, or alternatively, in a further option the third domain of the bispecific binding molecule according to this embodiment may comprise: (a) at least one variable heavy chain (V_{H}) polypeptide that comprises CDR 1, 2, and 3 sequences having the sequences of SEQ ID NOs: 138, 139, and 140, respectively, and optionally wherein the at least one variable heavy chain (V_{H}) polypeptide comprises, consists essentially of, or consists of, the sequence of SEQ ID NO: 130; and/or (b) at least one variable light chain (V_{L}) polypeptide that comprises CDR 1, 2 and 3 sequences having the sequences of SEQ ID NOs: 144, 145, and 146, respectively, and optionally wherein the variable light chain (V_{L}) polypeptide comprises, consists essentially of, or consists of, the sequence of SEQ ID NO: 134.

In one embodiment, the first domain of the bispecific binding molecule is an scFv comprising all six CDR sequences of OKT3 (preferably wherein the first domain is an scFv of OKT3), and the second domain of the bispecific binding molecule is an scFv comprising all six CDR sequences of 4H3C3, 7B1F3 and/or 2F5B11 (preferably wherein the second domain is an scFv of 4H3C3, 7B1F3 and/or 2F5B11), optionally wherein the first and second domain are connected via a linker. Optionally, a third domain is present which is an scFv comprising all six CDR sequences of 1D3, 1C10, 1A10, 1G9 and/or 1E8 (preferably wherein the second domain is an scFv of 1D3, 1C10, 1A10, 1G9 and/or 1E8), optionally wherein the third domain is connected to either, or both, of the first and second domains via a linker.

In one embodiment, the first domain of the bispecific binding molecule comprises two scFv each comprising all six CDR sequences of OKT3 (preferably wherein the first domain comprises two scFv of OKT3); and the second domain of the bispecific binding molecule comprises two Fab each comprising one, two, three, four, five or six CDRs corresponding to any one, two, three, four, five or all six of the CDR sequences of antibody (or humanised variants thereof as described herein):
(a) 4H3C3 as defined by SEQ ID NOs: 196, 197, 198, 199, 200 and 201 (preferably wherein the first domain comprises two Fab of 4H3C3);
(b) 7B1F3 as defined by SEQ ID NOs: 219, 220, 221, 199, 222 and 223 (preferably wherein the first domain comprises two Fab of 7B1F3); and/or
(c) 2F5B11 as defined by SEQ ID NOs: 242, 243, 244, 199, 245 and 246 (preferably wherein the first domain comprises two Fab of 2F5B11);
optionally wherein a third domain is present, wherein the third domain of the bispecific binding molecule comprises two Fab each comprising one, two, three, four, five or six CDRs corresponding to any one, two, three, four, five or all six of the CDR sequences of antibody:
(a) 1D3 as defined by SEQ ID NOs: 8, 9, 10, 14, 15 and 16 (preferably wherein the first domain comprises two Fab of 1D3);
(b) 1C10 as defined by SEQ ID NOs: 112, 113, 114, 117, 83 and 118 (preferably wherein the first domain comprises two Fab of 1C10);
(c) 1A10 as defined by SEQ ID NOs: 112, 113, 114, 117, 83 and 118 (preferably wherein the first domain comprises two Fab of 1A10);
(d) 1G9 as defined by SEQ ID NOs: 76, 77, 78, 82, 83 and 84 (preferably wherein the first domain comprises two Fab of 1G9); and/or
(e) 1E8 as defined by SEQ ID NOs: 76, 95, 96, 82, 99 and 100 (preferably wherein the first domain comprises two Fab of 1E8);
and further comprises an Fc, optionally wherein the Fc corresponds to the Fc of human IgG.

In some embodiments, the two Fab of the second and/or third domain are identical. In some embodiments, the two Fab of the second domain correspond to different antibodies selected from the group consisting of 4H3C3, 7B1F3 and 2F5B11. In some embodiments, the two Fab of the third domain correspond to different antibodies selected from the group consisting of 1D3, 1C10, 1A10, 1G9 and 1E8.

In one particular embodiment, the bispecific binding molecule is a BiTE having a CD3-binding OKT3 scFv domain binding region, and comprises a US28 ECD3-binding region having the sequence of the 1D3 antibody, and more specifically preferably comprises, consists essentially of, or consists of:
(i) a first polypeptide sequence comprising, consisting essentially of, or consisting of a heavy chain sequence that includes the V_{H} region of 1D3, optionally one or more C_{H} sequences (such as a C_{H}1, C_{H}2 and/or C_{H}3 sequence), an optional linker sequence, and the sequence of an OKT3 scFv, for example a heavy chain sequence having the sequence of SEQ ID NO: 58; and
(ii) a second polypeptide sequence comprising, consisting essentially of, or consisting of a light chain sequence that includes the V_{L} region of 4H3C3, and optionally a C_{L} sequence, for example a light chain sequence having the sequence of SEQ ID NO: 59.

In another embodiment, the sequence of the 4H3C3 antibody may be replaced in the BiTE by the corresponding sequences of an antibody selected from the group consisting of 7B1F3 and 2F5B11.

### CARs/CAR T Cells and Other Cells Recombinantly Expressing CARs

Immunotherapy-based treatments can be based on Chimeric Antigen Receptors (CARs). CARs are recombinant receptors for antigen, which, in a single molecule, redirect the specificity and function of T lymphocytes and other immune cells (Sadelain et al 2013 Cancer Discov 3(4): 388).

The general premise for their use in cancer immunotherapy is to rapidly generate tumour-targeted T cells, bypassing the barriers and incremental kinetics of active immunisation. Once expressed in T cells, CAR-modified T cells acquire supra-physiological properties and act as “living drugs” that may exert both immediate and long-term effects. The engineering of CARs into T cells requires that T cells be cultured to allow for transduction and expansion. The transduction may utilise a variety of methods, but stable gene transfer is required to enable sustained CAR expression in clonally expanding and persisting T cells.

In principle, any cell surface molecule can be targeted through a CAR, thus overriding tolerance to self-antigens and the antigen recognition gaps in the physiological T cell repertoire that limit the scope of T cell reactivity.

Various T cell subsets, as well as T cell progenitors and other immune cells such as natural killer cells, can be targeted with a CAR.

Adoptive immunotherapy using CAR engineered cells such as T cells is a promising approach in cancer treatment (Han et al 2013 J Hematol Oncol 6: 47). Significant progresses made in the past decades have contributed to the development of more efficient antitumour immunotherapy. For example, incorporation of a single chain variable fragment (scFv) of a tumour antigen specific antibody and signalling domains of T cell receptor renders CARs having the specificity of an antibody and the cytotoxicity of cytotoxic T lymphocytes. CARs endow T cells antigen specific recognition, activation and proliferation in an MHC independent manner. In addition, CAR bypasses many mechanisms through which cancer cells escape immunorecognition. These mechanisms include down-regulation of the MHC, reduced expression of costimulatory molecules, induction of suppressive cytokines and recruitment of regulatory T cells. Besides these beneficial effects, the technical feasibility of CARs make them even more attractive in the development of adoptive immunotherapy. The observations from preclinical and clinical studies have revealed a very encouraging therapeutic efficacy of CAR-mediated immunotherapy in a variety of cancers including lymphoma, chronic lymphocytic leukaemia, melanoma and neuroblastoma.

In one embodiment, the binding molecule of the first aspect of the present invention can be a CAR, for example a comprising:
(i) an extracellular domain, wherein the extracellular domain comprises or consists of a binding molecule (such as an antibody) as defined above, or a functional fragment of said binding molecule;
(ii) a transmembrane domain; and
(iii) an intracellular domain;
wherein the extracellular domain of the CAR has binding specificity to an epitope within ECD1 and/or ECD3 of a US28 protein of human cytomegalovirus (HCMV), and
wherein ECD1 and ECD3 of the US28 protein comprises an amino acid sequence presented in the US28 protein at positions corresponding to positions 1 to 37 and 167 to 183, respectively, of the US28 protein encoded by human cytomegalovirus (HCMV) as set forth in SEQ ID NO: 5.

Optionally:
(a) the extracellular domain of the CAR has binding specificity to an epitope present entirely within ECD1 and/or ECD3 of the US28 protein of HCMV;
(b) the extracellular domain of the CAR has binding specificity to a linear epitope within ECD1 and/or ECD3 of the US28 protein;
(c) the extracellular domain of the CAR has binding specificity to an epitope within ECD1 and/or ECD3 of a US28 protein of HCMV that is HCMV strain agnostic, for example, binding specificity to an epitope within ECD1 and/or ECD3 of a US28 protein of HCMV that is agnostic to two or more (such as all) of HCMV strains selected from the group consisting of D1B, Towne, AD169, BL, DAVIS, JP, Merlin, PH, TB40/E, Toledo, TR, VHL/E and VR1814 (FIX); and/or
(d) the extracellular domain of the CAR has specificity to an epitope within ECD1 and/or ECD3 of the US28 protein of HCMV, irrespective of whether the ECD3 of the US28 protein comprises the sequence of:
TKKDNQCMTDYDYLEVS (SEQ ID NO: 7) as found in ECD3 of US28 as encoded by a majority of HCMV strains, or
TKKNNQCMTDYDYLEVS (SEQ ID NO: 6) as found in ECD3 of US28 as encoded by a minority of HCMV strains.

In one preferred embodiment, the extracellular domain of the CAR may comprise one, two, three, four, five or six complementarity determining regions (CDRs) corresponding to any one, two, three, four, five or all six of the CDR sequences of antibody (or humanised variants thereof as described herein):
(a) 4H3C3 as defined by SEQ ID NOs: 196, 197, 198, 199, 200 and 201, respectively, and/or a functional variant of any one or more of said CDR sequences of antibody 4H3C3;
(b) 7B1F3 as defined by SEQ ID NOs: 219, 220, 221, 199, 222 and 223, respectively, and/or a functional variant of any one or more of said CDR sequences of antibody 7B1F3; and/or
(c) 2F5B11 as defined by SEQ ID NOs: 242, 243, 244, 199, 245 and 246, respectively, and/or a functional variant of any one or more of said CDR sequences of antibody 2F5B11;
and optionally further comprises one, two, three, four, five or six complementarity determining regions (CDRs) corresponding to any one, two, three, four, five or all six of the CDR sequences of antibody:
(d) 1D3 as defined by SEQ ID NOs: 8, 9, 10, 14, 15 and 16, respectively, and/or a functional variant of any one or more of said CDR sequences of antibody 1D3;
(e) 1C10 as defined by SEQ ID NOs: 112, 113, 114, 117, 83 and 118, respectively, and/or a functional variant of any one or more of said CDR sequences of antibody 1C10;
(f) 1A10 as defined by SEQ ID NOs: 112, 113, 114, 117, 83 and 118, respectively, and/or a functional variant of any one or more of said CDR sequences of antibody 1A10;
(g) 1G9 as defined by SEQ ID NOs: 76, 77, 78, 82, 83 and 84, respectively, and/or a functional variant of any one or more of said CDR sequences of antibody 1G9; and/or
(h) 1E8 as defined by SEQ ID NOs: 76, 95, 96, 82, 99 and 100, respectively, and/or a functional variant of any one or more of said CDR sequences of antibody 1E8.

Additionally, or alternatively, the extracellular domain of the CAR may comprise:
(a) one, two, or all three, of the CDR 1, 2, and 3, sequences of the variable heavy chain (V_{H}) of antibody:
   i. 4H3C3, as defined by SEQ ID NOs: 196, 197, and 198, respectively (or a functional variant of any one, two or three of said CDR sequences, such as a humanised variant thereof as described herein);
   ii. 7B1F3, as defined by SEQ ID NOs: 219, 220, and 221, respectively (or a functional variant of any one, two or three of said CDR sequences, such as a humanised variant thereof as described herein); and/or
   iii. 2F5B11, as defined by SEQ ID NOs: 242, 243, and 244, respectively (or a functional variant of any one, two or three of said CDR sequences);
      and optionally further comprise:
   iv. 1D3, as defined by SEQ ID NOs: 8, 9, and 10, respectively (or a functional variant of any one or more of said CDR sequences);
   v. 1C10, as defined by SEQ ID NOs: 112, 113, and 114, respectively (or a functional variant of any one or more of said CDR sequences);
   vi. 1A10, as defined by SEQ ID NOs: 112, 113, and 114, respectively (or a functional variant of any one or more of said CDR sequences);
   vii. 1G9, as defined by SEQ ID NOs: 76, 77, and 78, respectively (or a functional variant of any one or more of said CDR sequences); and/or viii. 1E8, as defined by SEQ ID NOs: 76, 95, and 96, respectively (or a functional variant of any one or more of said CDR sequences);
and/or
(b) one, two, or all three, of the CDR 1, 2, and 3, sequences of the variable light chain (V_{L}) of antibody:
   i. 4H3C3, as defined by SEQ ID NOs: 199, 200, and 201, respectively (or a functional variant of any one, two or three of said CDR sequences, such as a humanised variant thereof as described herein);
   ii. 7B1F3, as defined by SEQ ID NOs: 199, 222, and 223, respectively (or a functional variant of any one, two or three of said CDR sequences, such as a humanised variant thereof as described herein); and/or
   iii. 2F5B11, as defined by SEQ ID NOs: 199, 245, and 246, respectively (or a functional variant of any one, two or three of said CDR sequences);
      and optionally further comprise:
   iv. 1D3, as defined by SEQ ID NOs: 14, 15, and 16, respectively (or a functional variant of any one or more of said CDR sequences);
   v. 1C10, as defined by SEQ ID NOs: 117, 83, and 118, respectively (or a functional variant of any one or more of said CDR sequences);
   vi. 1A10, as defined by SEQ ID NOs: 117, 83, and 118, respectively (or a functional variant of any one or more of said CDR sequences);
   vii. 1G9, as defined by SEQ ID NOs: 82, 83, and 34, respectively (or a functional variant of any one or more of said CDR sequences);
   viii. 1E8, as defined by SEQ ID NOs: 82, 99, and 100, respectively (or a functional variant of any one or more of said CDR sequences).

Additionally, or alternatively, the extracellular domain of the CAR may comprise:
(a) at least one variable heavy chain (V_{H}) polypeptide sequence that comprises CDR 1, 2, and 3 sequences having the sequences of:
   i. SEQ ID NOs: 196, 197, and 198, respectively (or a functional variant of any one, two or three of said CDR sequences, such as a humanised variant thereof as described herein), and optionally wherein the at least one variable heavy chain (V_{H}) polypeptide sequence comprises, consists essentially of, or consists of, the sequence of SEQ ID NO: 187 (or a functional variant thereof, such as a humanised variant thereof as described herein);
   ii. SEQ ID NOs: 219, 220, and 221, respectively (or a functional variant of any one, two or three of said CDR sequences, such as a humanised variant thereof as described herein), and optionally wherein the at least one variable heavy chain (V_{H}) polypeptide sequence comprises, consists essentially of, or consists of, the sequence of SEQ ID NO: 211 (or a functional variant thereof, such as a humanised variant thereof as described herein); and/or
   iii. SEQ ID NOs: 242, 243, and 244, respectively (or a functional variant of any one, two or three of said CDR sequences), and optionally wherein the at least one variable heavy chain (V_{H}) polypeptide sequence comprises, consists essentially of, or consists of, the sequence of SEQ ID NO: 233 (or a functional variant thereof);
      and optionally of:
   iv. SEQ ID NOs: 8, 9, and 10, respectively (or a functional variant of any one or more of said CDR sequences), and optionally wherein the at least one variable heavy chain (V_{H}) polypeptide sequence comprises, consists essentially of, or consists of, the sequence of SEQ ID NO: 12 (or a functional variant thereof);
   v. SEQ ID NOs: 112, 113, and 114, respectively (or a functional variant of any one or more of said CDR sequences), and optionally wherein the at least one variable heavy chain (V_{H}) polypeptide sequence comprises, consists essentially of, or consists of, the sequence of SEQ ID NO: 104 (or a functional variant thereof);
   vi. SEQ ID NOs: 112, 113, and 114, respectively (or a functional variant of any one or more of said CDR sequences), and optionally wherein the at least one variable heavy chain (V_{H}) polypeptide sequence comprises, consists essentially of, or consists of, the sequence of SEQ ID NO: 122 (or a functional variant thereof);
   vii. SEQ ID NOs: 76, 77, and 78, respectively (or a functional variant of any one or more of said CDR sequences), and optionally wherein the at least one variable heavy chain (V_{H}) polypeptide sequence comprises, consists essentially of, or consists of, the sequence of SEQ ID NO: 68 (or a functional variant thereof); and/or
   viii. SEQ ID NOs: 76, 95, and 96, respectively (or a functional variant of any one or more of said CDR sequences), and optionally wherein the at least one variable heavy chain (V_{H}) polypeptide sequence comprises, consists essentially of, or consists of, the sequence of SEQ ID NO: 88 (or a functional variant thereof);
and/or
(b) at least one variable light chain (V_{L}) polypeptide sequence that comprises CDR 1, 2 and 3 sequences having the sequences of:
   i. SEQ ID NOs: 199, 200, and 201, respectively (or a functional variant of any one, two or three of said CDR sequences, such as a humanised variant thereof as described herein), and optionally wherein the at least one variable heavy chain (V_{L}) polypeptide sequence comprises, consists essentially of, or consists of, the sequence of SEQ ID NO: 189 (or a functional variant thereof, such as a hunmanised variant thereof as described herein);
   ii. SEQ ID NOs: 199, 222, and 223, respectively (or a functional variant of any one, two or three of said CDR sequences, such as a humanised variant thereof as described herein), and optionally wherein the at least one variable heavy chain (V_{L}) polypeptide sequence comprises, consists essentially of, or consists of, the sequence of SEQ ID NO: 213 (or a functional variant thereof, such as a humanised variant thereof as described herein); and/or
   iii. SEQ ID NOs: 199, 245, and 246, respectively (or a functional variant of any one, two or three of said CDR sequences), and optionally wherein the at least one variable heavy chain (V_{L}) polypeptide sequence comprises, consists essentially of, or consists of, the sequence of SEQ ID NO: 235 (or a functional variant thereof);
      and optionally of:
   iv. SEQ ID NOs: 14, 15, and 16, respectively (or a functional variant of any one or more of said CDR sequences), and optionally wherein the variable light chain (V_{L}) polypeptide comprises, consists essentially of, or consists of, the sequence of SEQ ID NO: 18 (or a functional variant thereof);
   v. SEQ ID NOs: 117, 83, and 118, respectively (or a functional variant of any one or more of said CDR sequences), and optionally wherein the at least one variable heavy chain (V_{H}) polypeptide sequence comprises, consists essentially of, or consists of, the sequence of SEQ ID NO: 108 (or a functional variant thereof);
   vi. SEQ ID NOs: 117, 83, and 118, respectively (or a functional variant of any one or more of said CDR sequences), and optionally wherein the at least one variable heavy chain (V_{H}) polypeptide sequence comprises, consists essentially of, or consists of, the sequence of SEQ ID NO: 126 (or a functional variant thereof);
   vii. SEQ ID NOs: 82, 83, and 84, respectively (or a functional variant of any one or more of said CDR sequences), and optionally wherein the at least one variable heavy chain (V_{H}) polypeptide sequence comprises, consists essentially of, or consists of, the sequence of SEQ ID NO: 72 (or a functional variant thereof); and/or
   viii. SEQ ID NOs: 82, 99, and 100, respectively (or a functional variant of any one or more of said CDR sequences), and optionally wherein the at least one variable heavy chain (V_{H}) polypeptide sequence comprises, consists essentially of, or consists of, the sequence of SEQ ID NO: 92 (or a functional variant thereof).

A functional variant of a reference sequence selected from SEQ ID NO: 187, 189, 211, 213, 233, 235, 12, 18, 68, 72, 88, 92, 104, 108, 122 or 126 may optionally have at least 70% sequence identity to the defined reference, for example at least 75%, 80%, 85%, 90%, 91%, 92%, 93%, 94%, 95%, 96%, 97%, 98% or 99% sequence identity, and more generally has 95-99% sequence identity to the defined amino acid sequence (e.g. 96%, 97%, 98% or 99% sequence identity). The variation may be solely within the non-CDR regions of the sequences of the functional variant of the variable heavy or light chain, solely within the CDR regions of the sequences, or within both the non-CDR and CDR regions of the sequences. It will be appreciated that, if variation is included in the CDR regions of the functional variant, then the variation may be within one CDR, two CDRs, or three CDRs, respectively of the heavy chain variable region, or the light chain variable region. Typically, the variation is outside of the CDR regions. A functional variant may be selected from SEQ ID NO: 256-271.

In certain embodiments, the extracellular domain of the CAR is, or comprises, consists essentially of, or consists of, the sequence of, an antibody, optionally a humanised antibody, for example a single-chain variable fragment (scFv) or a functional variant thereof, wherein said antibody and the functional variant thereof is a binding molecule according to the first aspect of the present invention.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its V_{L} and V_{H} regions. The linker may comprise any naturally occurring amino acid. In some embodiments, the linker sequence comprises amino acids glycine and serine. In another embodiment, the linker sequence comprises sets of glycine and serine repeats such as (Gly₄Ser)ᵣ, where n is a positive integer equal or greater than 1, such as 2, 3, 4, 5, 6 or more. In one embodiment, the linker is (Gly₄Ser)₃. Variation in the linker length may retain or enhance activity, giving rise to superior efficacy in activity studies.

The light chain variable region and heavy chain variable region of a scFv can be, for example, in any of the following orientations: light chain variable region-linker-heavy chain variable region or heavy chain variable region-linker-light chain variable region.

A CAR as described herein comprises a transmembrane domain. By a transmembrane domain we include the meaning of any moiety that is capable of being embedded in a lipid membrane. By being embedded in a lipid membrane we include the meaning of the transmembrane domain favourably interacting with the hydrophobic portions of the lipids that make up the lipid membrane. Insertion into lipid membranes may be assayed using any suitable method known in the art, including fluorescence labelling with fluorescence microscopy. Hence, it will be appreciated that the transmembrane domain is one that locates the CAR molecule within the lipid membrane.

Optionally, the transmembrane domain comprises the transmembrane domain of a protein (e.g. a transmembrane protein), for example the transmembrane domain of a transmembrane receptor protein. In an embodiment, the transmembrane domain is one that is associated with one of the other domains of the CAR. In an embodiment, the transmembrane domain comprises the transmembrane portion of an intracellular signalling protein that constitutes at least part of the intracellular signalling domain. In some instances, the transmembrane domain can be selected or modified by amino acid substitution to avoid binding of such domains to the transmembrane domains of the same or different surface membrane proteins, e.g. to minimise interactions with other members of the receptor complex. In some instances, the transmembrane domain is capable of homodimerisation with another CAR on the cell surface.

The transmembrane domain may be derived either from a natural or from a recombinant source. The domain may be derived from any membrane-bound or transmembrane protein. In one embodiment, the transmembrane domain is capable of signalling to the intracellular domain(s) whenever the CAR has bound to a target. A suitable transmembrane domain for use in the invention may include the transmembrane region(s) of the alpha, beta or zeta chain of the T cell receptor, CD28, CD3 epsilon, CD8, CD45 and CD4.

The transmembrane domain can, for example, include one or more additional amino acids adjacent to the transmembrane region, such as one or more amino acids associated with the extracellular region of the protein from which the transmembrane domain was derived (e.g. 1, 2, 3, 4, 5, 6, 7, 8, 9, 10 up to 15 amino acids of the extracellular region) and/or one or more additional amino acids associated with the intracellular region of the protein from which the transmembrane protein is derived (e.g. 1, 2, 3, 4, 5, 6, 7, 8, 9, 10 up to 15 amino acids of the intracellular region). In certain embodiments, the extracellular domain of the CAR is connected to the transmembrane domain by a hinge region. The hinge region may comprise one or more immunoglobulin domains. Particular examples include the Fc region of IgG1 and the immunoglobulin-like extracellular regions of CD4 and CD8. It will be appreciated that when the antibody that selectively binds US28 is a scFv molecule, the hinge region can be used to extend the reach of the scFv to allows its attachment to the transmembrane domain without affecting its binding to US28. The hinge may be from a human protein such as human immunoglobulin. The hinge region may, for example, be a CD8α hinge region (for example, as described in An et al, 2016, Oncotarget, 7:10638-10649, the contents of which are incorporated herein by reference).

Typically, the transmembrane domain comprises predominantly hydrophobic amino acid residues such as leucine and valine.

In an embodiment, a short oligo- or polypeptide linker, such as between 2 and 10 amino acids in length, may form the linkage between the transmembrane domain and the intracellular signalling domain of the CAR. The linker may, for example, comprise glycine and/or serine residues. An example of a suitable linker is a glycine-serine doublet.

By intracellular signalling domain we include the meaning of a domain that is capable of activating at least one of the normal functions of the cell in which the CAR is introduced, such as at least one of the normal effector functions of an immune cell (e.g. T cell). An effector function refers to a specialised function of a cell. The effector function of a T cell, for example, may be cytolytic function or helper activity including the secretion of cytokines. Thus, the intracellular signalling domain may be a portion of a protein which transduces the effector function signal and directs the cell (e.g. T cell) to perform a specialised function.

Generally, the whole intracellular signalling domain can be used; however, it is appreciated that it is not necessary to use the entire domain, provided that whatever part of the signalling domain that is used is still capable of transducing the effector function signal. It will also be appreciated that variants of such intracellular signalling domains with substantially the same or greater functional capability may also be used. By this we include the meaning that the variants should have substantially the same or greater transduction of the effector functional signal. Typically, substantially the same or greater signal transduction includes at least 80%, 85%, 90%, 95%, 100%, 105%, 110%, 115%, or 120%, or more of the signal transduction of the unmodified intracellular signalling domain, wherein signal transduction of the unmodified intracellular signalling domain corresponds to 100%.

Methods for assessing transduction of effector function signal are well known to those skilled in the art and include, for example, assessing the amounts and/or activity of molecules (e.g. proteins such as cytokines) that are indicative of the transduced signal. Thus, when the signal is the cytolytic function of a T-cell, the methods may involve measurement of one or more cytokines secreted by the T-cell, which cytokines are known to have a cytolytic activity (e.g. IFN gamma) and following measurement of the target cell lysis.

Examples of intracellular signalling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are generally insufficient for full activation of a T cell and that a secondary and/or costimulatory signal may also be required. Thus, T cell activation can be said to be mediated by two distinct classes of intracellular signalling sequences: those that initiate antigen-dependent primary activation through the TCR (primary intracellular signalling domains) and those that act in an antigen-independent manner to provide a secondary or costimulatory signal (secondary intracellular signalling domain, such as a costimulatory domain). Costimulatory domains promote activation of effector functions and may also promote persistence of the effector function and/or survival of the cell.

A primary intracellular signalling domain regulates primary activation of the TCR complex either in a stimulatory way, or in an inhibitory way. Primary intracellular signalling domains that act in a stimulatory manner may contain signalling motifs which are known as immunoreceptor tyrosine-based activation motifs or ITAMs (e.g. 2, 3, 4, 5 or more ITAMs). Thus, the intracellular signalling domain may comprise one or more ITAMs. Examples of ITAM containing primary intracellular signalling domains that are of particular use in the invention include those of CD3 zeta, Fc receptor gamma, Fc receptor beta, CD3 gamma, CD3 delta, CD3 epsilon, CD5, CD22, CD79a, CD79b, and CD66d.

In one embodiment, a CAR of the invention comprises an intracellular signalling domain of CD3-zeta.

It will be appreciated that one or more ITAMs of the intracellular signalling domain may be modified, for example by mutation. The modification may be used to increase or decrease the signalling function of the ITAM as compared with the native ITAM domain.

As mentioned above, the intracellular signalling domain may comprise a primary intracellular signalling domain by itself, or it may comprise a primary intracellular signalling domain in combination with one or more secondary intracellular signalling domains, such as one or more costimulatory signalling domains. Thus, the intracellular signalling domain of the CAR may comprise the CD3 zeta signalling domain by itself or in combination with one or more other intracellular signalling domains such as one or more costimulatory signalling domains.

The costimulatory signalling domain refers to a portion of the CAR comprising the intracellular domain of a costimulatory molecule. A costimulatory molecule may be a cell surface molecule other than an antigen receptor or its ligands that is required for an efficient response of immune cells (e.g. lymphocytes) to an antigen. Examples of such molecules include CD28, 4-1BB (CD137), OX40, ICOS, DAP10, CD27, CD30, CD40, PD1, ICOS, lymphocyte function-associated antigen-1 (LFA-1), CD2, CD7, LIGHT, NKG2C, B7-H3, and a ligand that specifically binds with CD83, and the like. For example, CD27 co-stimulation has been demonstrated to enhance expansion, effector function, and survival of human CAR T cells in vitro and augments human T cell persistence and anti-tumour activity in vivo (Song et al., Blood. 2012, 119(3):696-706).

The intracellular signalling sequences within the intracellular portion of the CAR of the invention may be linked to each other in a random or specified order. Optionally, a short oligo- or polypeptide linker, for example, between 2 and 10 amino acids (e.g. 2, 3, 4, 5, 6, 7, 8, 9, or 10 amino acids) in length may form the linkage between intracellular signalling sequences. In one embodiment, a glycine-serine doublet can be used as a suitable linker. In another embodiment, a single amino acid, such as an alanine or a glycine, can be used as a suitable linker.

In one embodiment, the intracellular signalling domain is designed to comprise two or more, for example 3, 4, 5, or more, costimulatory signalling domains. In an embodiment, the two or more, e.g. 2, 3, 4, 5, or more, costimulatory signalling domains, are separated by a linker molecule, such as one described herein. In one embodiment, the intracellular signalling domain comprises two costimulatory signalling domains. In some embodiments, the linker molecule is a glycine residue. In some embodiments, the linker is an alanine residue.

In a preferred embodiment, the intracellular portion of the CAR comprises the signalling domain of CD3 zeta and the signalling domain of CD28.

In another embodiment, the intracellular portion of the CAR comprises the signalling domain of CD3-zeta and the signalling domain of 4-1BB.

In another embodiment, the intracellular portion of the CAR comprises the signalling domain of CD3-zeta and the signalling domain of OX40.

In another embodiment, the intracellular portion of the CAR comprises the signalling domain of CD3-zeta and the signalling domain of ICOS.

In another embodiment, the intracellular portion of the CAR comprises the signalling domain of CD3-zeta and the signalling domain of DAP10.

In another embodiment, the intracellular portion of the CAR comprises the signalling domain of CD3-zeta, the signalling domain of 4-1BB and the signalling domain of OX40.

### Other Optional Components of CAR

In an embodiment, the CAR may further comprise a leader sequence. By a “leader sequence” we include the meaning of a peptide sequence that can direct the CAR to the cell membrane. Thus, when the CAR is a chimeric fusion protein, it may contain a leader sequence at the amino-terminus (N-terminus). Optionally, the leader sequence is cleaved from the CAR during cellular processing and localisation of the CAR to the cellular membrane.

In a further embodiment, the CAR may comprise an inducible suicide moiety. By an inducible suicide moiety, we include the meaning of a molecule which possesses an inducible capacity to lead to the death of the cell in whose cellular membrane the CAR resides (e.g. T cell). In this way, the effect that the CARs have on a subject can be tightly controlled via selective deletion of the cells that comprise them. Conveniently, the suicide moiety comprises the epitope of an antibody that is either directly or indirectly cytotoxic. Antibodies that are directly cytotoxic include lytic antibodies such as Rituximab, which binds to CD20. Thus, in one embodiment, the CAR may comprise a CD20 epitope. Antibodies may also be indirectly cytotoxic by being conjugated to one or more cytotoxic moieties.

In one preferred embodiment, the CAR comprises: (i) an extracellular domain, wherein the extracellular domain comprises, consists essentially of, or consists of, an antibody according to the first aspect of the present invention that selectively binds to ECD3 of the US28 polypeptide (e.g. a scFv fragment); (ii) a transmembrane domain, and (iii) an intracellular signalling domain (e.g. an intracellular signalling domain comprising a primary signalling domain such as CD3 zeta, and optionally one or more costimulatory domains such as CD28, 4-1BB, OX40, ICOS and DAP10).

### Fusion Polypeptides:

In a further option, a binding molecule as defined by the first aspect of the present invention may comprises a fusion polypeptide sequence, wherein the fusion polypeptide sequence comprises a first amino acid sequence fused a second amino acid sequence, and wherein the first amino acid sequence comprises or consists of at least one of the polypeptide chains of the binding molecule or of the functional fragment thereof, and the second amino acid sequence is a fusion partner.

Any fusion partner of interest may be selected. The skilled person is well aware of fusion partner sequences known in the art, and any may be selected. A fusion partner may, for example, provide an additional or alternative binding property; it may provide an effector portion that creates an effect at the site of binding of the binding molecule (examples include a sequence that is able to amplify the immune response or induce direct damage to any cell that is bound by the binding molecule, e.g. a cytotoxic sequence); it may provide for a modulation (such as an increase or decrease) in the circulatory half-life of the binding molecule; it may provide a sequence that facilities the capture, recovery or purification of the binding molecule; and/or it may provide a sequence that facilitates the detection of the binding molecule.

### Conjugates:

Binding molecules according to the first aspect of the present invention may include, and/or be linked to, at least one agent to form a conjugate, such as an antibody conjugate. Said agent may optionally be non-proteinaceous, such as a small molecule drugs or other agents, which for example, may be low molecular weight (<900 Daltons) organic compounds.

In order to increase the efficacy of antibody molecules, and other binding agents, as diagnostic or therapeutic agents, it is conventional to link or covalently bind or complex at least one desired molecule or moiety. Such a molecule or moiety may be, but is not limited to, at least one effector or reporter molecule. Effector molecules may comprise molecules having a desired activity, e.g., drugs, cytotoxic agents, immunosuppressive agents, anti-inflammatory agents, etc. Such molecules can be optionally attached to the antibody molecule, or other binding agent, via a cleavable linker designed to allow the molecules to be released at or near the target site.

Said desired molecule or moiety may optionally be selectively active in an extracellular environment or selectively active in an intracellular environment. As discussed above, US28 is a GCPR, of which trafficking to the plasma membrane allows both its direct targeting with binding molecules and its use as a transporter of payload due to its endocytosis, which is either constitutive or occurs as a result of ligand binding. GCPRs in general constitute the largest family of proteins targeted by approved drugs (Sriram & Insel, Mol Pharmacol, 2018, 93(4): 251-258). Accordingly, binding molecules of the present invention, which have binding specificity to the ECD1 (and optionally ECD3) of US28, can be used to bring about the internalization, by a US28-expressing cell (in particular, a HCMV-infected cell), of one or more desired molecule or moiety, such as a desired molecule or moiety that is selectively active in an intracellular environment, by conjugation of said one or more desired molecule or moiety to a binding molecule of the present invention.

By contrast, a reporter molecule is defined as any moiety which may be detected using an assay. Non-limiting examples of reporter molecules which have been conjugated to antibodies, and other binding agents, include enzymes, radiolabels, haptens, fluorescent labels, phosphorescent molecules, chemiluminescent molecules, chromophores, photoaffinity molecules, colored particles or ligands, such as biotin.

Such conjugates are commonly for use as diagnostic agents. These diagnostic agents generally fall within two classes, those for use in in vitro diagnostics, such as in a variety of immunoassays and/or immunohistochemistry (IHC), and those for use in vivo diagnostic protocols, generally known as “antibody-directed imaging.” Many appropriate imaging agents are known in the art, as are methods for their attachment to antibodies and other such binding molecules (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰, ¹²⁵I is often being preferred for use in certain embodiments, and technicium^{99m} and/or indium¹¹¹ are also often preferred due to their low energy and suitability for long range detection. Radioactively labeled monoclonal antibodies may be produced according to well-known methods in the art. For instance, monoclonal antibodies and other binding agents can be iodinated by contact with sodium and/or potassium iodide and a chemical oxidizing agent such as sodium hypochlorite, or an enzymatic oxidizing agent, such as lactoperoxidase. Monoclonal antibodies and other binding agents may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups are often used to bind radioisotopes to an antibody, or other binding agent, and exist as metallic ions are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of conjugates contemplated are those intended primarily for use in vitro, where the antibody, or other binding molecule, is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Several methods are known in the art for the attachment or conjugation of an antibody, or other binding molecule, to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies, or other binding molecules, may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins, or other binding molecules, by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature.

### Pro-drugs:

It will be appreciated that the binding molecules of the first aspect of the invention may be administered in a “prodrug” form. For example, in a prodrug form, the ECD1- and/or ECD3-binding portion of the binding molecule which selectively binds to ECD1 or ECD3, respectively, of US28 may be masked in such a way that is selectively unmasked only in the locality of target cells (e.g. those which express US28 and/or are latently and/or lytically infected HCMV).

The term “prodrug” as used in this application refers to a precursor or derivative form of a biologically or pharmaceutically active substance that is less active compared to the parent biologically or pharmaceutically active substance and is capable of being enzymatically activated or converted into the more active parent form (see, for example, D. E. V. Wilman “Prodrugs in Cancer Chemotherapy” Biochemical Society Transacti^{on}s 14, 375-382 (615th Meeting, Belfast 1986) and V. J. Stella et al., “Prodrugs: A Chemical Approach to Targeted Drug Delivery” Directed Drug Delivery R. Borchardt et al., (ed.) pages 247-267 (Humana Press 1985)).

### B. Nucleic Acid Molecules

A second aspect of the present invention provides a nucleic acid molecule, or combination of multiple distinct nucleic acid molecules, wherein the nucleic acid molecule comprises, or the combination of multiple distinct nucleic acid molecules collectively comprise, one or more nucleic acid sequences that, individually or in combination, encode the one or more polypeptide chains provided by a binding molecule of the first aspect of the present invention.

The, or each, nucleic acid molecule in accordance with the second aspect of the present invention may, for example, be DNA (e.g. genomic DNA or complementary DNA) or RNA (e.g. a mRNA molecule, an in vitro transcribed RNA or a synthetic RNA). In one embodiment, the, or each, nucleic acid molecule is a cDNA molecule. It may comprise deoxyribonucleotides, ribonucleotides, modified nucleotides or bases, and/or their analogues, or any substrate that can be incorporated into a polymer by DNA or RNA polymerase, or by a synthetic reaction. A polynucleotide may comprise modified nucleotides, such as methylated nucleotides and their analogues. If present, modification to the nucleotide structure may be imparted before or after assembly of the polymer. The sequence of nucleotides may be interrupted by non-nucleotide components. Some specific examples of nucleic acid molecules encoding antibodies of the invention are described herein. Other suitable sequences can readily be determined based upon the knowledge of antibody structure and the genetic code.

By the term, “in vitro transcribed RNA” we include the meaning of RNA, including mRNA, that has been synthesised in vitro. Generally, the in vitro transcribed RNA is generated from an in vitro transcription vector or a PCR-generated polynucleotide template. The in vitro transcription vector comprises a template that is used to generate the in vitro transcribed RNA. A method for generating mRNA for use in transfection can involve in vitro transcription of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length. Such RNA can be used to efficiently transfect different kinds of cells, as described further below.

A nucleic acid molecule, or combination of multiple distinct nucleic acid molecules, in accordance with the second aspect of the present invention may be isolated. By “isolated” we include the optional meaning that the nucleic acid molecule is not located or otherwise provided within a cell.

The, or each, nucleic acid molecule in accordance with the second aspect of the present invention may single stranded. Alternatively the, or each, nucleic acid molecule in accordance with the second aspect of the present invention may be double stranded.

In the instance that the binding molecule of the first aspect of the present invention is formed from a single continuous amino acid sequence, then it may be encoded by a single nucleic acid molecule coding sequence in accordance with the present invention.

In the instance that the binding molecule of the first aspect of the present invention is formed from a two or more separate polypeptide sequences, then each separate polypeptide sequence may be encoded by a separate nucleic acid molecule such that the totality of the binding molecule is encoded by combination of multiple distinct nucleic acid molecules; alternatively, the two or more separate polypeptide sequences of the binding molecule may be encoded by a single nucleic acid molecule comprising multiple distinct coding sequences, wherein those multiple distinct coding respectively sequences encode each of the different two or more separate polypeptide sequences of the binding molecule.

In one embodiment, a nucleic acid molecule in accordance with the second aspect of the present invention encodes a binding molecule or the first aspect of the present invention, or a part thereof, comprising an antibody heavy chain or variable region thereof.

In one embodiment, a nucleic acid molecule in accordance with the second aspect of the present invention encodes a binding molecule or the first aspect of the present invention, or a part thereof, comprising an antibody light chain or variable region thereof.

In one embodiment, a nucleic acid molecule in accordance with the second aspect of the present invention, or a combination of multiple distinct nucleic acid molecules in accordance with the second aspect of the invention collectively, encodes a binding molecule or the first aspect of the present invention, or a part thereof, comprising both an antibody heavy chain or variable region thereof and an antibody light chain or variable region thereof.

Hence, it will be appreciated that the nucleic acid molecule, or combination of multiple distinct nucleic acid molecules, may encode any of the binding molecules (such as antibodies, fragments thereof, fusion proteins, CARs etc.) of the first aspect of the present invention, including variants of the particular amino acid sequences as defined above, or parts thereof.

In one embodiment, a nucleic acid molecule in accordance with the second aspect of the present invention comprises (or a combination of multiple distinct nucleic acid molecules in accordance with the second aspect of the invention collectively comprises):
a) a nucleotide sequence encoding a variable heavy chain sequence comprising the sequences of:
   i. SEQ ID NOs: 190, 191 and 192 encoding the V_{H}-CDR1, V_{H}-CDR2 and V_{H}-CDR3 sequences of 4H3C3, respectively;
   ii. SEQ ID NOs: 214, 215 and 216 encoding the V_{H}-CDR1, V_{H}-CDR2 and V_{H}-CDR3 sequences of 7B1F3, respectively;
   iii. SEQ ID NOs: 236, 237 and 238 encoding the V_{H}-CDR1, V_{H}-CDR2 and V_{H}-CDR3 sequences of 2F5B11, respectively;
      and optionally of:
   iv. SEQ ID NOs: 22, 23 and 24 encoding the V_{H}4-CDR1, V_{H}-CDR2 and V_{H}-CDR3 sequences of 1D3, respectively;
   v. SEQ ID NOs: 109, 110 and 111 encoding the V_{H}-CDR1, V_{H}-CDR2 and V_{H}-CDR3 sequences of 1C10, respectively;
   vi. SEQ ID NOs: 109, 110 and 111 encoding the V_{H}-CDR1, V_{H}-CDR2 and V_{H}-CDR3 sequences of 1A10, respectively;
   vii. SEQ ID NOs: 73, 74 and 75 encoding the V_{H}-CDR1, V_{H}-CDR2 and V_{H}-CDR3 sequences of 1G9, respectively; and/or
   viii. SEQ ID NOs: 73, 93 and 94 encoding the V_{H-CDR}1, V_{H}-CDR2 and V_{H}-CDR3 sequences of 1E8, respectively;
and
b) a nucleotide sequence encoding a variable light chain sequence comprising the sequences of:
   i. SEQ ID NOs: 193, 194 and 195 encoding the V_{L}-CDR1, V_{L}-CDR2 and V_{L}-CDR3 sequences of 4H3C3, respectively;
   ii. SEQ ID NOs: 251, 217 and 218 encoding the V_{L}-CDR1, V_{L}-CDR2 and V_{L}-CDR3 sequences of 7B1F3, respectively;
   iii. SEQ ID NOs: 239, 240 and 241 encoding the V_{L}-CDR1, V_{L}-CDR2 and V_{L}-CDR3 sequences of 2F5B11, respectively;
      and optionally of:
   iv. SEQ ID NOs: 28, 29 and 30 encoding the V_{L}-CDR1, V_{L}-CDR2 and V_{L}-CDR3 sequences of 1D3, respectively;
   v. SEQ ID NOs: 115, 80 and 116 encoding the V_{H}-CDR1, V_{H}-CDR2 and V_{H}-CDR3 sequences of 1C10, respectively;
   vi. SEQ ID NOs: 115, 80 and 116 encoding the V_{H}-CDR1, V_{H}-CDR2 and V_{H}-CDR3 sequences of 1A10, respectively;
   vii. SEQ ID NOs: 79, 80 and 81 encoding the V_{H}-CDR1, V_{H}-CDR2 and V_{H}-CDR3 sequences of 1G9, respectively; and/or
   viii. SEQ ID NOs: 79, 97 and 98 encoding the V_{H}-CDR1, V_{H}-CDR2 and V_{H}-CDR3 sequences of 1E8, respectively;
or a functional variant of any of these sequences, for example a variant comprising 1, 2, 3, 4 or 5 nucleotide substitutions within any one or more of said SEQ ID NOs, and/or a variant having at least 70%, 75%, 80%, 85%, 90% or 95 sequence identity to said SEQ ID Nos. For example, the or each variant nucleic acid change may be a silent mutation, and therefore not change the sequence of the amino acid sequence that it encodes.

In another embodiment, a nucleic acid molecule in accordance with the second aspect of the present invention comprises (or a combination of multiple distinct nucleic acid molecules in accordance with the second aspect of the invention collectively comprises):
a) a nucleotide sequence comprising sequence selected from the group consisting of SEQ ID NOs: 183, 207, 229, 26, 102, 120, 66 and 86, encoding the variable heavy chain sequence of4H3C3, 7B1F3, 2F5B11, 1D3, 1C10, 1A10, 1G9 and IES, respectively, and
b) a nucleotide sequence comprising sequence selected from the group consisting of SEQ ID NOs: 185, 209, 231, 32, 106, 124, 70 and 90, encoding the variable light chain sequence of 4H3C3, 7B1F3, 2F5B11, 1D3, 1C10, 1A10, 1G9 and 1E8, respectively,
or a functional variant of any of these sequences, for example a variant having at least 20%, 30%, 40%, 50%, 60%, 70%, 75%, 80%, 85%, 90% or 95% sequence identity to said SEQ ID Nos.

In another embodiment, a nucleic acid molecule in accordance with the second aspect of the present invention comprises (or a combination of multiple distinct nucleic acid molecules in accordance with the second aspect of the invention collectively comprises):
a) a nucleotide sequence comprising sequence of:
   i. SEQ ID NO: 178 (with or without the leader sequence-encoding region as indicated and, if without, then optionally comprising a sequence encoding an alternative leader sequence), encoding the complete mature sequence of the heavy chain of 4H3C3, as defined by SEQ ID NO: 180;
   ii. SEQ ID NO: 202 (with or without the leader sequence-encoding region as indicated and, if without, then optionally comprising a sequence encoding an alternative leader sequence), encoding the complete mature sequence of the heavy chain of 7B1F3, as defined by SEQ ID NO: 204;
   iii. SEQ ID NO: 224 (with or without the leader sequence-encoding region as indicated and, if without, then optionally comprising a sequence encoding an alternative leader sequence), encoding the complete mature sequence of the heavy chain of 2F5B11, as defined by SEQ ID NO: 226;
      and optionally of:
   iv. SEQ ID NO: 34 (with or without the leader sequence-encoding region as indicated and, if without, then optionally comprising a sequence encoding an alternative leader sequence), encoding the complete mature sequence of the heavy chain of 1D3, as defined by SEQ ID NO: 20;
   v. SEQ ID NO: 155 (with or without the leader sequence-encoding region as indicated and, if without, then optionally comprising a sequence encoding an alternative leader sequence), encoding the complete mature sequence of the heavy chain of 1C10, as defined by SEQ ID NO: 157;
   vi. SEQ ID NO: 159 (with or without the leader sequence-encoding region as indicated and, if without, then optionally comprising a sequence encoding an alternative leader sequence), encoding the complete mature sequence of the heavy chain of 1A10, as defined by SEQ ID NO: 161;
   vii. SEQ ID NO: 147 (with or without the leader sequence-encoding region as indicated and, if without, then optionally comprising a sequence encoding an alternative leader sequence), encoding the complete mature sequence of the heavy chain of 1G9, as defined by SEQ ID NO: 149; and/or
   viii. SEQ ID NO: 151 (with or without the leader sequence-encoding region as indicated and, if without, then optionally comprising a sequence encoding an alternative leader sequence), encoding the complete mature sequence of the heavy chain of 1E8, as defined by SEQ ID NO: 153;
   and
b) a nucleotide sequence comprising sequence of:
   i. SEQ ID NO: 179 (with or without the leader sequence-encoding region as indicated and, if without, then optionally comprising a sequence encoding an alternative leader sequence), encoding the complete mature sequence of the heavy chain of 4H3C3, as defined by SEQ ID NO: 181;
   ii. SEQ ID NO: 203 (with or without the leader sequence-encoding region as indicated and, if without, then optionally comprising a sequence encoding an alternative leader sequence), encoding the complete mature sequence of the heavy chain of 7B1F3, as defined by SEQ ID NO: 205;
   iii. SEQ ID NO: 225 (with or without the leader sequence-encoding region as indicated and, if without, then optionally comprising a sequence encoding an alternative leader sequence), encoding the complete mature sequence of the heavy chain of 2F5B11, as defined by SEQ ID NO: 227;
      and optionally of:
   iv. SEQ ID NO: 35 (with or without the leader sequence-encoding region as indicated and, if without, then optionally comprising a sequence encoding an alternative leader sequence), the complete mature sequence of the light chain of 1D3 as defined by SEQ ID NO: 21;
   v. SEQ ID NO: 156 (with or without the leader sequence-encoding region as indicated and, if without, then optionally comprising a sequence encoding an alternative leader sequence), encoding the complete mature sequence of the light chain of 1C10, as defined by SEQ ID NO: 158;
   vi. SEQ ID NO: 160 (with or without the leader sequence-encoding region as indicated and, if without, then optionally comprising a sequence encoding an alternative leader sequence), encoding the complete mature sequence of the light chain of 1A10, as defined by SEQ ID NO: 162;
   vii. SEQ ID NO: 148 (with or without the leader sequence-encoding region as indicated and, if without, then optionally comprising a sequence encoding an alternative leader sequence), encoding the complete mature sequence of the light chain of 1G9, as defined by SEQ ID NO: 150; and/or
   viii. SEQ ID NO: 152 (with or without the leader sequence-encoding region as indicated and, if without, then optionally comprising a sequence encoding an alternative leader sequence), encoding the complete mature sequence of the light chain of 1E8, as defined by SEQ ID NO: 154;
or a functional variant of any of these sequences, for example a variant having at least 20%, 30%, 40%, 50%, 60%, 70%, 75%, 80%, 85%, 90% or 95% sequence identity to said SEQ ID Nos (optionally, wherein the sequence identity is determined in respect of said sequences without the leader sequence-encoding regions as indicated).

A functional variant of said defined nucleic acid sequences, in accordance with the foregoing embodiments may, for example, be a substitution, deletion and/or addition variant of any of the above nucleic acid sequences. A variant polynucleotide may comprise 1, 2, 3, 4, 5, up to 10, up to 20, up to 30, up to 40, up to 50, up to 75 or more nucleic acid substitutions and/or deletions from the sequences given in the sequence listing.

In some embodiments, variants may be at least 70% homologous to a polynucleotide of any one of nucleic acid sequences disclosed herein, preferably at least 80% or 90% and more preferably at least 95%, 97% or 99% homologous thereto. Preferably homology and identity at these levels is present at least with respect to the coding regions of the polynucleotides. Methods of measuring homology are well known in the art and it will be understood by those of skill in the art that in the present context, homology is calculated on the basis of nucleic acid identity. Such homology may exist over a region of at least 15, preferably at least 30, for instance at least 40, 60, 100, 200 or more contiguous nucleotides. Such homology may exist over the entire length of the unmodified polynucleotide sequence.

Optionally, in some variant nucleic acid sequences described herein, the sequences of the regions encoding any one or more of the six CDR sequences present within the variable heavy and variable light chains maybe conserved, and any sequence variations present elsewhere; or in a further option the level and/or nature of variation in the regions encoding any one or more of the six CDR sequences present within the variable heavy and variable light chains may be minimised compared to the rest of the, or each nucleic acid molecule. For example, the extent of variation within the or each of the regions encoding any one or more of the six CDR sequences present within the variable heavy and variable light chains may be limited to 1, 2, 3, 4 or 5 nucleotide substitutions and/or present a CDR coding region having at least 70%, 75%, 80%, 85%, 90% or 95% sequence identity to the corresponding non-variant CDR coding region. Variations in the or each variant nucleic acid change may, for example, be a silent mutation, and therefore not change the sequence of the amino acid sequence that it encodes; this may be (although not necessary) particularly preferred in the regions that encode any one or more of the V_{H}-CDR1, V_{H}-CDR2 and V_{H}-CDR3 sequences and/or the V_{L}-CDR1, V_{L}-CDR2 and V_{L}-CDR3 sequences.

Methods of measuring polynucleotide homology or identity are known in the art. For example, the UWGCG Package provides the BESTFIT program which can be used to calculate homology (e.g. used on its default settings) (Devereux et al., 1.984, Nucleic Acids Research 12: 387-395, the disclosures of which are incorporated herein by reference).

The PILEUP and BLAST algorithms can also be used to calculate homology or line up sequences (typically on their default settings), for example as described in Altschul, 1993, J Mol Evol 36:290-300; Altschul et al., 1990, J Mol Biol 215:403-10, the disclosures of which are incorporated herein by reference).

Software for performing BLAST analysis is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff & Henikoff, 1992) alignments (B) of 50, expectation (E) of 10, M=5, N=4, and a comparison of both strands.

The BLAST algorithm performs a statistical analysis of the similarity between two sequences; see e.g. Karlin & Altschul, 1993. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologue may differ from a sequence in the relevant polynucleotide by less than 3, 5, 10, 15, 20 or more mutations (each of which may be a substitution, deletion or insertion). These mutations may be measured over a region of at least 30, for instance at least 40, 60 or 100 or more contiguous nucleotides of the homologue.

In one embodiment, a variant sequence may vary from the specific sequences given in the sequence listing by virtue of the redundancy in the genetic code. The DNA code has 4 primary nucleic acid residues (A, T, C and G) and uses these to “spell” three letter codons which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons. A variant polynucleotide of the invention may therefore encode the same polypeptide sequence as another polynucleotide of the invention, but may have a different nucleic acid sequence due to the use of different codons to encode the same amino acids.

One or more polypeptides present within a binding molecule of the first aspect of the present invention may thus be produced from or delivered in the form of one or more polynucleotides which encodes, and is capable of expressing, it or them.

Polynucleotides of the invention can be synthesised according to methods well known in the art, as described by way of example in Green & Sambrook (2012, Molecular Cloning—a laboratory manual, 4^{th} edition; Cold Spring Harbor Press).

It will be appreciated by persons skilled in the art that the nucleic acid molecule may be codon-optimised for expression of the antibody polypeptide in a particular host cell, e.g. for expression in human cells (for example, see Angov, 2011).

For the avoidance of doubt, where variants of nucleic acids that encode particular CDR sequences are mentioned, it will be appreciated that one or more of the nucleotide sequences encoding the CDRs as defined may be varied. Thus, where the nucleic acid is defined as encoding heavy chain or light chain CDRs (e.g. CDRs 1-3), each being encoded by a particular nucleic acid sequence, up to one, two or three of those particular sequences may be varied and so on. Similarly, where the nucleic acid is defined as encoding light chain and heavy chain CDRs (e.g. six CDRs), each being encoded by a particular nucleotide sequence, up to one, two, three, four, five or all six of those particular sequences may be varied. The variants are typically ones which lead to conservative amino acid substitutions as described above.

Where the nucleic acid is defined as encoding a light chain variable region and a heavy chain variable region, it will be appreciated that up to one or two of those sequences may be varied as defined. The variation may be solely within the part of the sequence encoding non-CDR regions, solely within the part of the sequence encoding CDR regions, or within both parts that encode CDR regions and parts that encode non-CDR regions. Typically, the variation is outside of the CDR regions, and so the nucleic acid may comprise any of the nucleic acids that encode the particular CDRs defined herein (e.g. in SEQ ID NOs: 8, 9, 10, 14, 15 and/or 16 for 1D3, or any of the corresponding SEQ ID NOs in 4H3C3, 7B1F3, 2F51B11, 1C10, 1A10, 1G9 and/or 1E8).

It will be appreciated that any variant of the specific nucleic acid sequences described herein should not significantly affect one or more of the desired binding properties, for example the selective binding specificity to ECD1 and/or ECD3 of US28, the ability to bind specifically but in a strain agnostic manner to ECD1 and/or ECD3 of US28 and/or the binding affinity to ECD1 and/or ECD3 of US28. Methods of testing these desired activities are described above in relation to the first aspect of the invention.

In a particularly preferred embodiment, the nucleic acid encodes (or the combination of multiple distinct nucleic acid molecules collectively encode) a binding molecule of the first aspect of the present invention that is an scFv that selectively binds to ECD1 and/or ECD3 of US28. Thus, the nucleic acid (or combination of multiple distinct nucleic acid molecules) may comprise regions of sequence that, respectively, encode a variable heavy (V_{H}) chain sequence and a variable light (V_{L}) chain sequence, typically in the form of a single continuous nucleic acid coding region, for example a single continuous nucleic acid coding region in which the regions of sequence encoding the V_{H} and V_{L} sequences are linked by a sequence encoding a linker sequence, such that the scFv can be expressed as a single polypeptide encoded by a single nucleic acid sequence.

In another particularly preferred embodiment, the nucleic acid encodes (or the combination of multiple distinct nucleic acid molecules collectively encode) a binding molecule of the first aspect of the present invention is a bispecific immune cell engager antibody, for example, a bispecific T-cell engager (BiTE). For example, the nucleic acid (or one or more of the combination of multiple distinct nucleic acid molecules) may encode a region that is a cell-engaging regions, such as a T-cell engaging region, for example a CD3-binding domain.

In another particularly preferred embodiment, the nucleic acid encodes (or the combination of multiple distinct nucleic acid molecules collectively encode) a binding molecule of the first aspect of the present invention that is a chimeric antigen receptor (CAR). Accordingly, the nucleic acid molecule, or combination of multiple distinct nucleic acid molecules, according to the second aspect of the present invention may include one or more nucleic acid sequences encoding a CAR that comprises:
(i) an extracellular domain (for example an antibody, such as an scFv), wherein the extracellular domain comprises or consists of a binding molecule of the first aspect of the present invention as defined herein, or a functional fragment of said binding molecule as defined herein;
(ii) a transmembrane domain (for example, the transmembrane domain of a transmembrane receptor protein, and optionally wherein the transmembrane domain comprises the transmembrane domain of a protein selected from the group consisting of the alpha, beta or zeta chain of the T-cell receptor, CD28, CD3 epsilon, CD8, CD45 and CD4); and
(iii) an intracellular domain (e.g., an intracellular signalling domain, for example wherein: (a) the intracellular signalling domain comprises one or more immunoreceptor tyrosine-based activation motifs (ITAMs); and/or (b) the intracellular signalling domain comprises a signalling domain of CD3 zeta, Fc receptor gamma, Fc receptor beta, CD3 gamma, CD3 delta, CD3 epsilon, CD5, CD22, CD79a, CD79b, and CD66d);
wherein the extracellular domain of the CAR has binding specificity to a first epitope within ECD1 (and optionally a second epitope within ECD3) of a US28 protein of human cytomegalovirus (HCMV), and
wherein ECD1 and ECD3 of the US28 protein comprises an amino acid sequence presented in the US28 protein at positions corresponding to positions 1 to 37 and 167 to 183, respectively, of the US28 protein encoded by human cytomegalovirus (HCMV) as set forth in SEQ ID NO: 5.

Optionally, the extracellular domain of a CAR encoded by the nucleic acid molecule(s) of the second aspect of the present invention may, for example, be connected to the transmembrane domain by a hinge region. Accordingly, the nucleic acid molecule(s) of the second aspect of the present invention may include a region of nucleic acid sequence encoding said hinge region.

The intracellular domain of a CAR encoded by the nucleic acid molecule(s) of the second aspect of the present invention may, for example, be connected to the transmembrane domain by a hinge region for example, comprise one or more costimulatory domains, for example: (a) wherein the one or more costimulatory domains includes one or more functional signalling domains obtained from a protein selected from the group consisting of CD28, 41BB, OX40, ICOS, CD27, and DAP10; (b) wherein the intracellular domain incorporates a costimulatory domain proximal to the intracellular signalling domain, (c) wherein the intracellular domain comprises two or more costimulatory domains, for example two in-line costimulatory domains, and/or (d) wherein the intracellular domain incorporates separate cytokine signals. Accordingly, the nucleic acid molecule(s) of the second aspect of the present invention may include one or more regions of nucleic acid sequence encoding said one or more costimulatory domains.

The, or each, nucleic acid molecule in accordance with the second aspect of the present invention may also encode leader sequences, transmembrane domains, intracellular signalling domains, and hinge regions, such as any one or more of such sequences as described above. Thus, the or each, nucleic acid molecule in accordance with the second aspect of the present invention may comprise a leader sequence (also referred to as a signal peptide) (e.g. of SEQ ID Nos: 253, 13 or 19).

Optionally, the nucleic acid molecule, or combination of multiple distinct nucleic acid molecules, according to the second aspect of the present invention may be provided (collectively, or individually in separate form) in the form of an expression cassette which includes control sequences operably linked to the inserted sequence, thus allowing for expression of the polypeptide of the invention in vivo. Such an expression cassette may be administered directly to a host subject. These expression cassettes, in turn, are typically provided within vectors (e.g., plasmids or recombinant viral vectors), for example as discussed further below.

### C. Vectors

The third aspect of the present invention further provides a vector comprising a nucleic acid molecule, or combination of multiple distinct nucleic acid molecules, according to the second aspect of the present invention.

The term “vector” refers to a nucleic acid which incorporates the sequence of one or more isolated nucleic acid sequences and which can be used to deliver the, or each, isolated nucleic acid to the interior of a cell. Numerous vectors are known in the art including linear polynucleotides, polynucleotides associated with ionic or amphiphilic compounds, plasmids, and viruses. Thus, the term vector includes an autonomously replicating plasmid or a virus. The term should also be construed to further include non-plasmid and non-viral compounds which facilitate transfer of nucleic acid into cells, such as, for example, a polylysine compound, liposome, and the like.

In the context used herein, the term “vector” includes single and/or multiple vectors. For example, it may include multiple vectors in the context that a binding molecule of the first aspect of the present invention is encoded by combination of multiple distinct nucleic acid molecules, according to the second aspect of the present invention, whereby one or more of the nucleic acids present in said combination of multiple distinct nucleic acid molecules can be presented on a first vector, and one or more of the other nucleic acids present in said combination of multiple distinct nucleic acid molecules can be presented on a second distinct vector, and/or yet other nucleic acids in said combination of multiple distinct nucleic acid molecules can be presented on third, fourth, fifth, etc. distinct further vectors.

Thus, for example, if the binding molecule of the first aspect of the present invention is an antibody or CAR that comprises more than one distinct polypeptide chains, then optionally a single form of vector according to the third aspect of the present invention may comprise all necessary nucleic acid sequences to encode the more than one distinct polypeptide chains that form the binding molecule. Alternatively, the third aspect of the present invention also provides a combination of multiple distinct vectors (e.g. two vectors, or more), wherein the combination of the multiple distinct vectors, collectively, comprise a combination of multiple distinct nucleic acid molecules according to the second aspect of the present invention. Thus, for example, if the binding molecule of the first aspect of the present invention is an antibody or CAR that comprises more than one distinct polypeptide chains, then the multiple distinct vectors may each comprise one or more nucleic acid sequences that each encode different and distinct polypeptide chains that collectively form the binding molecule, such that the binding molecule comprises one or more distinct polypeptide chains encoded by a first vector and one or more other distinct polypeptide chains encoded by a one or more subsequent different vectors.

For the sake of brevity, in general the present application refers to “vector” in the singular, although it is to be understood that this can optionally also encompass “vectors”, including multiple distinct forms of vector as described above, in the plural. Optionally, the (or each) vector may be selected from the group consisting of a retroviral vector, a plasmid, a lentivirus vector, and an adenoviral vector.

The (or each) vector according to the third aspect of the present invention, comprising a nucleic acid molecule, or combination of multiple distinct nucleic acid molecules, according to the second aspect of the present invention, may be administered to a host subject. Preferably the polynucleotide is prepared and/or administered using a genetic vector. A suitable vector may be any vector which is capable of carrying a sufficient amount of genetic information, and allowing expression of one or more polypeptide sequences encoded by the, or each, vector, such as allowing the expression of a binding molecule according to the first aspect of the present invention.

The expression of natural or synthetic nucleic acids encoding binding molecules of the first aspect of the present invention (such as antibodies or fragments thereof, including scFvs, BiTEs and the like, well as CARs) is typically achieved by operably linking one or more nucleic acids, each independently encoding one or more polypeptides (or portions thereof), to a promoter, and incorporating the or each construct into one or more expression vectors. Thus, the (or each) vector may be an expression vector.

The third aspect of present invention thus includes expression vectors that comprise such polynucleotide sequences. By “expression vector” we include the meaning of a vector comprising a recombinant polynucleotide comprising expression control sequences operatively linked to a nucleotide sequence to be expressed. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of one or more polypeptide sequences encoded by the, or each, vector, such as allowing the expression of a binding molecule according to the first aspect of the present invention. Other suitable vectors would be apparent to persons skilled in the art (see Green & Sambrook, supra).

An expression vector may comprise sufficient cis-acting elements for expression; other elements for expression can be supplied by the host cell or in an in vitro expression system. Expression vectors include all those known in the art, including cosmids, plasmids (e.g. naked or contained in liposones) and viruses (e.g. lentiviruses, retroviruses, adenoviruses, and adeno-associated viruses) that incorporate the recombinant polynucleotide. Typical cloning vectors contain transcription and translation terminators, initiation sequences, and promoters useful for regulation of the expression of the desired nucleic acid sequence.

Further, the expression vector may be provided in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY). Viruses, which are useful as vectors include, but are not limited to, retroviruses, adenoviruses, adeno-associated viruses, herpes viruses, and lentiviruses.

A number of viral based systems have been developed for gene transfer into mammalian cells. For example, retroviruses provide a convenient platform for gene delivery systems. One or more selected nucleic acid sequences, encoding one or more polypeptide sequences of interest, can be inserted into a vector and packaged in retroviral particles using techniques known in the art. The recombinant virus can then be isolated and delivered to cells either in vivo or ex vivo. A number of retroviral systems are known in the art.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Lentiviral vectors are particularly suitable tools to achieve long-term gene transfer since they allow long-term, stable integration of a transgene and its propagation in daughter cells.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g. the LENTIVECTOR® gene delivery technology from Oxford BioMedica, the LENTIMAX™ vector system from Lentigen and the like. Non-clinical types of lentiviral vectors are also available and would be known to one skilled in the art.

Adenovirus vectors may also be used, and a number of adenovirus vectors are known in the art.

Hybrid vectors may also be used, and a number of hybrid vectors are known in the art. Hybrid vectors generally include vector viruses that are genetically engineered to have qualities of more than one vector. Viruses are altered to avoid the shortcomings of typical viral vectors, which may have limited loading capacity, immunogenicity, genotoxicity, and fail to support long-term adequate transgenic expression.

The expression of nucleic acids encoding a binding molecule according to the first aspect of the present invention can also be accomplished using transposons such as sleeping beauty, CRISPR, CAS9, and zinc finger nucleases.

The vector can be suitable for replication and integration in eukaryotes, and/or it may be suitable for expression in prokaryotes, such as in bacterial species. Preferably, the, or each, vector, is capable of expressing a binding molecule according to the first aspect of the present invention a production cell (e.g. a CHO cell, an E. coli cell, etc.) or in the cell of a subject, for example in mammalian cells (e.g. human cells), such as mammalian (e.g. human) immune cells (e.g. T cells, NK cells, macrophages, etc.).

The nucleic acid molecule, or combination of multiple distinct nucleic acid molecules, according to the second aspect of the present invention can also be cloned into a number of types of vectors including a plasmid, a phagemid, a phage derivative, an animal virus, and a cosmid. Vectors of particular interest include expression vectors, replication vectors, probe generation vectors, and sequencing vectors.

In general, a suitable vector will optionally contain an origin of replication functional in at least one organism, a promoter sequence operably linked to the, or each, encoding nucleic acid sequence, convenient restriction endonuclease sites, and/or one or more selectable markers, (e.g. WO 01/96584; WO 01/29058; and U.S. Pat. No. 6,326,193).

It will be appreciated that a vector according to the third aspect of the present invention may comprise additional promoter elements, e.g. enhancers, which regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, the individual elements can function either cooperatively or independently to activate transcription.

An example of a promoter that is capable of expressing a transgene of interest in a mammalian T cell is the EFla promoter. The native EFla promoter drives expression of the alpha subunit of the elongation factor-1 complex, which is responsible for the enzymatic delivery of aminoacyl tRNAs to the ribosome. The EFla promoter has been extensively used in mammalian expression plasmids and has been shown to be effective in driving expression from transgenes (e.g. CAR-encoding transgenes) cloned into a lentiviral vector. See, e.g., Milone et al., Mol. Ther. 17(8): 1453-1464 (2009).

Another example of a promoter is the immediate early cytomegalovirus (CMV) promoter sequence. This promoter sequence is a strong constitutive promoter sequence capable of driving high levels of expression of any polynucleotide sequence operatively linked thereto.

Other constitutive promoter sequences may, alternatively or additionally, be used, including, but not limited to the simian virus 40 (SV40) early promoter, mouse mammary tumour virus (MMTV), human immunodeficiency virus (HIV) long terminal repeat (LTR) promoter, MoMuLV promoter, an avian leukaemia virus promoter, an Epstein-Barr virus immediate early promoter, a Rous sarcoma virus promoter, as well as human gene promoters such as, but not limited to, the actin promoter, the myosin promoter, the elongation factor-Ia promoter, the haemoglobin promoter, and the creatine kinase promoter.

Further, the invention is not limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Thus, it will be appreciated that in any of the methods or uses of the invention described herein, the expression of a binding molecule according to the first aspect of the present invention, encoded by the nucleic acid(s) of the second aspect of the present invention or the vector(s) according to the third aspect of the present invention, may be conditional or inducible, for example, through environmental stimulus or through the action of a second molecule. In this way, it may be possible to limit or minimise adverse off-target effects (e.g. in tissues that do not express, or express low levels of, US28).

In order to assess the expression of the polypeptide(s) of interest in a cell (for example, a binding molecule according to the first aspect of the present invention), the vector may conveniently contain either a selectable marker gene or a reporter gene or both, so as to facilitate identification and selection of expressing cells. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g. enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (GFP) (e.g. Ui-Tei et al., 2000 FEES Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

The expression constructs of the present invention may also be used for nucleic acid immunisation and gene therapy, using standard gene delivery protocols (see, for example, U.S. Pat. No. 5,399,346). Hence, the vector may be a gene therapy vector. In this case, it may be desirable to link the nucleic acid and/or vector to a suitable targeting moiety so that it can be directed to an appropriate cell, tissue or organ for expression. Thus, it will be appreciated that in certain embodiments the nucleic acid molecule and/or the vector of the invention may be used in the therapeutic aspects of the invention via a gene therapy approach, using the formulations and methods described below and known in the art.

### D. Transformed Cells

A fourth aspect of the present invention provides a cell, or a population of cells (optionally a homogeneous or heterogeneous population of cells) comprising the nucleic acid molecule, or combination of multiple distinct nucleic acid molecules, according to the second aspect of the present invention, and/or a vector according to the third aspect of the present invention, optionally wherein the cell expresses one or more binding molecules according to the first aspect of the present invention (such as one or more antibodies, e.g. a monoclonal antibody, and/or one or more CARs), said one or more binding molecules and/or CARs being encoded by the nucleic acid molecule, or combination of multiple distinct nucleic acid molecules, according to the second aspect of the present invention, or a vector according to the third aspect of the present invention. Said cells may, optionally, be selected from isolated cells, ex vivo cells, and in vitro cells.

Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors or expression cassettes encoding for a binding molecule according to the first aspect of the present invention include mammalian HEK293T, CHO, HeLa, NSO and COS cells. Preferably the cell line selected will be one which is not only stable, but also allows for mature glycosylation and/or cell surface expression of a polypeptide.

Such cell lines of the invention may be cultured using routine methods to produce binding molecule according to the first aspect of the present invention, or may be used therapeutically or prophylactically to deliver binding molecule according to the first aspect of the present invention to a subject.

Alternatively, polynucleotides, expression cassettes or vectors of the invention may be administered to a cell from a subject ex vivo and the cell then returned to the body of the subject.

In the embodiment that said cell(s) are used therapeutically or prophylactically with a subject, then the cell may optionally be “autologous” or “allogeneic”, with respect to the subject, as described further below.

A cell according to the fourth aspect of the present invention may, for example, comprise: (a) a nucleic acid molecule, or combination of multiple distinct nucleic acid molecules, according to the second aspect of the present invention, wherein the encoded binding molecule is an antibody, a functional fragment of said antibody, or an antibody of functional fragment thereof that comprises a fusion polypeptide sequence, according to the first aspect of the present invention; and/or (b) a vector according to the third aspect of the present invention, wherein said vector comprises a nucleic acid molecule, or combination of multiple distinct nucleic acid molecules as defined by option (a) of this paragraph.

A cell according to the fourth aspect of the present invention may, for example, comprise: (a) a nucleic acid molecule, or combination of multiple distinct nucleic acid molecules, according to the second aspect of the present invention, wherein the encoded binding molecule is a CAR according to the first aspect of the present invention; and/or (b) a vector according to the third aspect of the present invention, wherein said vector comprises a nucleic acid molecule, or combination of multiple distinct nucleic acid molecules as defined by part (a) of this paragraph. Without limitation, said cell may, for example, be selected from the group consisting of: a T cell, natural killer (NK) cell, and a macrophage. Accordingly, the cell may optionally be a CAR-T cell, a CAR-NK cell or a CAR-macrophage, and optionally, when the cell is a CAR-T cell, then for example the T-cell may be selected from the group consisting of CD8⁺ T cells, CD4+ T cells, effector T cells, helper T cells, memory T cells, cytotoxic T lymphocytes (CTLs), EBV-specific T cell receptor (TCR) or γδ-T cell subtypes.

The fourth aspect of the present invention also provides a cell comprising a binding molecule according the first aspect of the present invention and/or a nucleic acid encoding said binding molecule, optionally wherein said nucleic acid is a nucleic acid or vector as defined by the second or third aspects of the present invention, respectively. For example, the binding molecule may be an antibody according the first aspect of the present invention, a functional fragment of said antibody according the first aspect of the present invention, or an antibody of functional fragment thereof that comprises a fusion polypeptide sequence according the first aspect of the present invention, and optionally wherein the antibody is monoclonal antibody, and further for example wherein the cell is mammalian cell, such as a CHO cell, that recombinantly expresses the monoclonal antibody.

The fourth aspect of the present invention also provides a cell comprising a CAR according to the first aspect of the present invention and/or a nucleic acid encoding said CAR, optionally wherein said nucleic acid is a nucleic acid or vector as defined by the second or third aspects of the present invention, respectively. Without limitation, said cell may, for example, be selected from the group consisting of: a T cell, natural killer (NK) cell, and a macrophage (M). Accordingly, the cell may optionally be a CAR-T cell, a CAR-NK cell or a CAR-M cell, and optionally, when the cell is a CAR-T cell, then for example the T-cell may be selected from the group consisting of CD8⁺ T cells, CD4+ T cells, effector T cells, helper T cells, memory T cells, cytotoxic T lymphocytes (CTLs), EBV-specific T cell receptor (TCR) or γδ-T cell subtypes.

A fifth aspect of the present invention provides a method of producing a cell, more particularly a recombinant cell, or a population of such cells (optionally a homogeneous or heterogeneous population of cells), the method comprising introducing a nucleic acid molecule, or combination of multiple distinct nucleic acid molecules, according to the second aspect of the present invention, and/or a vector according to third aspect of the present invention, into a cell.

Methods of introducing and expressing genes into a cell are known in the art. In the context of an expression vector, the vector can be readily introduced into a host cell, e.g. mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g. human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362. A polynucleotide of interest may be delivered to a cell through viral transduction.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. Typically, a liposome (e.g. an artificial membrane vesicle) may be used as a delivery vehicle in vitro and in vivo. Other methods of state-of-the-art targeted delivery of nucleic acids are available, such as delivery of polynucleotides with targeted nanoparticles or other suitable sub-micron sized delivery system.

In the case where a non-viral delivery system is utilised, the delivery vehicle may be a liposome. The use of lipid formulations is contemplated for the introduction of the nucleic acids into a host cell (in vitro, ex vivo or in vivo). The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidyiglycerol (“DMPG”) and other lipids may be obtained from Avanti Polar Lipids, Inc. (Birmingham, AL.).

“Liposome” is a generic term encompassing a variety of single and multilamellar lipid vehicles formed by the generation of enclosed lipid bilayers or aggregates. Liposomes can be characterised as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as non-uniform aggregates of lipid molecules. Also contemplated are lipofectamine-nucleic acid complexes.

One or more RNA molecules encoding a binding molecule of the first aspect of the present invention can be introduced to a cell using a form of transient transfection. Other methods of introducing encoding RNA(a) to a cell include, but are not limited to, for example, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al., Hum Gene Ther., 12(8):861-70 (2001).

Regardless of the method used to introduce exogenous nucleic acids into a host cell or otherwise expose a cell to the nucleic acid or vector of the invention, in order to confirm the presence of the nucleic acid in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g. by immunological means (ELISAs and Western blots) or by assays described herein to identify agents falling within the scope of the invention.

Said method optionally further comprises a step of selecting cells according to the fifth aspect of the present invention; for example selecting said cells from a heterogeneous cell population, thereby to create an enriched and/or homogeneous cell population. Said selection step may include selecting for the presence of one or more selectable markers present in the nucleic acid molecule, or combination of multiple distinct nucleic acid molecules, according to the second aspect of the present invention, and/or a vector according to third aspect of the present invention.

Said method optionally comprises culturing said cells. Said cultured cells may consequently produce one or more binding molecules according to the first aspect of the present invention, and said method may include the isolation and/or purification of the one or binding molecules from said cultured cells. Optionally, the isolated and/or purified one or more binding molecules may be formulated, for example in a pharmaceutically acceptable formulation, and/or administered to a subject in need thereof.

### E. CAR-Expressing Cells:

As discussed above, in a preferred embodiment, a cell according to the fourth aspect of the present invention may comprise:
(a) a nucleic acid molecule, or combination of multiple distinct nucleic acid molecules, according to the second aspect of the present invention, wherein the encoded binding molecule is a CAR according to the first aspect of the present invention; and/or
(b) a vector according to the third aspect of the present invention, wherein said vector comprises a nucleic acid molecule, or combination of multiple distinct nucleic acid molecules as defined by part (a) of this paragraph.

Without limitation, said cell may, for example, be selected from the group consisting of: a T cell, natural killer (NK) cell, and a macrophage (M). Accordingly, the cell may optionally be a CAR-T cell, a CAR-NK cell or a CAR-M cell.

When the cell is a CAR-T cell, then the T cell may be any of an alpha-beta T cell, a gamma-delta T cell, a memory T cell (e.g. a memory T cell with stem cell-like properties). For example the T-cell may be selected from the group consisting of CD8⁺ T cells, CD4+ T cells, effector T cells, helper T cells, memory T cells, cytotoxic T lymphocytes (CTLs), EBV-specific T cell receptor (TCR) or γδ-T cell subtypes. In a particularly preferred embodiment, the immune cell can be a memory T cell with stem cell like properties.

When the cell is a CAR-NK cell, then the NK cell may optionally be an invariant NK cell.

When the cell is a CAR-macrophage (CAR-M) cell, then the macrophage may optionally be an MI-type macrophage. As discussed in Mukhopadhyay (Nature Methods, 2020, 17: 559-565), although the expression of CARs on T cells has shown enormous promise in the treatment of malignancies, CAR-T cell therapy can be hampered by the inability of T cells to penetrate solid tumors, as well as the inhibitory tumor microenvironment (TME), whereas it has been shown that the introduction of CARs into macrophages (CAR-Ms) can efficiently contribute to an antitumor response.

However, more generally, it will be appreciated that a CAR-expressing cell may be any type of cell (e.g. an immune cell such as a T-cell) derived from a subject (e.g. a human subject), or any cell line. Such cells may be modified to comprise the nucleic acid or vector according to the invention and thereby express a CAR of the invention. Thus, the invention includes a method of producing a cell expressing a CAR of the invention, the method comprising obtaining a cell derived from a subject or providing a cell derived from a subject or providing a cell line, and introducing one or more polynucleotide molecules according to the second aspect of the invention, or a vector according to the third aspect of the invention, into said cell or cell line.

Examples of subjects include mammals, humans, dogs, cats, mice, rats and transgenic species thereof.

The cell may be “autologous” or “allogeneic”, as described further below.

By “autologous” we include the meaning that the CAR-expressing cell is derived from cells which originate from an individual to whom the CAR-expressing cells are to be used in accordance with the various aspects of the present application.

By “allogeneic” we include the meaning that the CAR-expressing cell is derived from cells which do not originate from the individual to whom the CAR-expressing cells are to be used in accordance with the various aspects of the present application. Typically, the cells are derived from cells of the same species as the individual on which the methods or uses are to be carried out.

Immune cells such as T cells can be obtained from a number of sources peripheral blood mononuclear cells, bone marrow, lymph node tissue, cord blood, thymus tissue, tissue from a site of infection, ascites, pleural effusion, spleen tissue, and tumors. Any number of cell lines (e.g. immune cell lines such as T cell lines) available in the art, may also be used.

In an embodiment, immune cells (e.g. T cells) are obtained from a unit of blood collected from a subject using any suitable techniques known in the art such as Ficoll™ separation. In another embodiment, cells from the circulating blood of a subject are obtained by apheresis. The apheresis product typically contains lymphocytes, including T cells, monocytes, granulocytes, B cells, other nucleated white blood cells, red blood cells, and platelets. It will be appreciated that the cells collected by apheresis may be washed to remove the plasma fraction and to place the cells in an appropriate buffer or media for subsequent processing steps. For example, the cells may be washed with phosphate buffered saline (PBS). Alternatively, the wash solution lacks calcium and may lack magnesium or may lack many if not all divalent cations. Initial activation steps in the absence of calcium can lead to magnified activation. A washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed, and the cells directly resuspended in culture media.

In an embodiment, T cells are isolated from peripheral blood lymphocytes by lysing the red blood cells and depleting the monocytes, for example, by centrifugation through a PERCOLL™ gradient or by counter-flow centrifugal elutriation. Specific subpopulations of T cells, such as CD3+, CD28+, CD4+, CD8+, CD45RA+, and CD45RO+ T cells, may be further isolated by positive or negative selection techniques known in the art. For example, T cells may be isolated by incubation with anti-CD3/anti-CD28 (e.g. 3×28)-conjugated beads, such as DYNABEADS® M-450 CD3/CD28 T, for a time period sufficient for positive selection of the desired T cells. Additionally or alternatively, a population of T cells may be enriched by negative selection, for instance by a combination of antibodies directed to surface markers unique to the negatively selected cells. Cell sorting and/or selection via negative magnetic immunoadherence or flow cytometry may be used.

It will be understood that cells derived from subjects that are to be modified to express the CAR (and/or other binding molecule) of the invention may be stored for a period of time prior to their use (see, for example, therapeutic methods below). For example, the cells may be frozen, optionally after they have been washed, or they may be incubated under suitable conditions for them to remain viable until needed (e.g. on a rotator at 2-10° C. or at room temperature). In this way, the cells can be stored until such time as they might be needed. They may be stored in an unmodified state (i.e. wherein they do not express the CAR of the invention) or in a modified state (i.e. wherein they have been modified to express the CAR of the invention).

Prior to use in the therapeutic applications described further below, the cells may be activated and expanded generally using methods known in the art. For example, T cells may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g. bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9): 13191328, 1999; Garland et al., J. Immunol Meth. 227(1-2):53-63, 1999).

In an embodiment, the cell that expresses a CAR (and/or other binding agent) of the invention is further modified to comprise or express one or more other agents that enhance the activity of the cell (e.g. T cell).

For example, the other agent may be an agent that inhibits an inhibitory molecule that is known to decrease the ability of the CAR-expressing cell to mount an effective immune response. Examples of inhibitory molecules include PD1, PD-L1, CTLA4, TIM3, LAG3, VISTA, BTLA, TIGIT, LAIR1, CD160, 2B4 and TGF-beta receptor. The agent that inhibits the inhibitory molecule may comprise a first polypeptide, e.g. an inhibitory molecule, associated with a second polypeptide that provides a positive signal to the cell, e.g. an intracellular signalling domain described herein.

Additionally or alternatively, the other agent may be a pro-inflammatory or pro-proliferative cytokine. The purpose of such cytokines may be to provide autocrine support to enhance the function, proliferation and/or persistence of CAR-expressing cells, and/or favourably alter the tumour microenvironment and recruit endogenous innate and cognate immune effects.

It will be appreciated that the invention provides a population of cells that comprise one or more polynucleotides according to the second aspect of the invention and/or one or more vectors according to the fourth aspect of the invention. Thus, the invention provides a population of cells that express the CAR (and/or other binding molecule) of the invention (e.g. immune cells such as T cells). In some embodiments, the population of CAR expressing cells comprise a mixture of cells expressing different CARs. For example, the population of cells may comprise a first cell expressing a CAR having binding domain with specificity for the ECD1 and/or ECD3 of US28 as described herein, and a second cell expressing a CAR having a different binding domain, such as an anti-US28 binding domain, for example a different CAR that has specificity for the ECD1 and/or ECD3 of US28 as described herein (for example, wherein each cell targets a distinct epitope of ECD1 or ECD3, or wherein one cell targets an epitope of ECD1 and another cell targets an epitope of ECD3). As another example, the population of CAR expressing cells can include a first cell expressing a CAR that includes binding domain with specificity for the ECD1 of US28 as described herein, and a second cell expressing a CAR that includes an antigen binding domain to a target other than US28, optionally with a third cell expressing a CAR that includes binding domain with specificity for the ECD3 of US28 as described herein. As yet another example, the population of CAR expressing cells can include a first cell expressing a CAR that includes a binding domain with specificity for the ECD1 of US28 as described herein, and a second cell expressing one or more agents that enhance the activity of the CAR expressing cell (e.g. T cell), optionally with a third cell expressing a CAR that includes binding domain with specificity for the ECD3 of US28 as described herein.

### Assaying CAR Activity

The invention also includes methods for making a CAR of the invention. For example, the invention comprises expressing in a suitable host cell a recombinant vector encoding the CAR, and recovering the CAR. Methods for expressing and purifying polypeptides (e.g. antibodies) are very well known in the art.

Once a CAR of the invention has been constructed, various assays can be used to assess its activity including assaying the ability to expand T cells following antigen stimulation, sustain T cell expansion in the absence of re-stimulation, and anti-cancer activities in appropriate in vitro and in vivo (e.g. animal) models.

In vitro expansion of CAR-expressing cells (e.g. T cells) following antigen stimulation can be measured by flow cytometry. For example, the cells may express the CAR along with a fluorescent reporter, and fluorescence measured in the presence and absence of US28, and/or in the presence or absence of one or more peptides according to the thirteenth and/or fourteenth aspects of the present invention. Suitable methods are described in Milone et al., Molecular Therapy 17(8): 1453-1464 (2009).

Animal models can also be used to measure an activity of a CAR expressing cell (e.g. immune cell such as a T cell). For example, a xenograft model using US28-specific CAR-expressing T cells may be used to treat a primary human US28-expressing cancer in immunodeficient mice, as has been clone for CD19-specific CAR-expressing T cells in treating pre-B ALL in immunodeficient mice (see, for example Milone et al., Molecular Therapy 17(8): 1453-1464 (2009)).

Also, the activity of CARs may be determined by assessing cell proliferation and cytokine production (see, for example Milone et al., Molecular Therapy 17(8): 1453-1464 (2009)).

Imaging technologies can also be used to evaluate specific trafficking and proliferation of CARs in tumor-bearing animal models. Such assays have been described, for example, in Barrett et al., Human Gene Therapy 22: 1575-1586 (2011).

Other assays, including those that are known in the art can also be used to evaluate the CAR constructs of the invention.

Agents Useful in Combination with CAR-Expressing Cell Therapies:

The present invention also contemplates the use of CAR-expressing cells in combination with an agent that increases the efficacy, and/or proliferation, and/or persistence of said CAR-expressing cell. Preferably the agent that increases the efficacy, and/or proliferation, and/or persistence of a CAR-expressing cell is a cytokine.

The further therapeutic agent may be a cytokine to enhance the efficacy/persistence/expansion of the CAR-expressing cells (e.g. T cells).

The further therapeutic agent may be an agent that ameliorates one or more side effects associated with administration of a CAR-expressing cell. For example, the further therapeutic agent may be used to treat “cytokine storm” (e.g. anti-IL6 antibody). By “cytokine storm” (also known as cytokine cascade and hypercytokinemia) we mean a potentially fatal hyper-release of inflammatory mediators (cytokines) in response to stimulation of T cells and macrophages by pathogens and immune insults.

The further therapeutic agent may be an antibody, or combination of antibodies, that block CTLA-4 (e.g. ipilimumab and tremelimumab), PD-1 (e.g. nivolumab, pembrolizumab and pidilizumab) and/or PD-L1 (e.g. MDX-1105 and MPDL3280A).

It has been confirmed that CAR-T cell therapy could be directed to the tumor tissues through the co-expression of chemokine receptors (CXCR2 or CCR4) or through combination with chemokines (Di Stasi et al., 2009, Blood, 113:6392-6402; Kershaw et al., 2002, Hum Gene Ther., 13:1971-1980). This could be a choice since the US28 also binds to multiple CC chemokines as well as CX3CR1.

Xia et al., 2016, Cancer Res. 76:6747-6759 suggested that combining an oncolytic virus with CAR-T cell therapy may be particularly efficacious in stimulator of interferon genes protein-inactivated and type I IFN-disrupted tumors. By contrast, Ajina and Maher (J Immunother Cancer., 2017; 5:90), Kim (Nat Commun. 2017; 8:344) and Scott et al (Macromol Biosci., 2018 January; 18) indicated that oncolytic virus infection might augment entry and mobilization of CAR-T cells, and mitigate or reverse local immunosuppression and enhance the function and persistence of CAR-T cell effectors.

### F. Production and Purification of Binding Molecules

A sixth aspect of the present invention provides a method of producing a binding molecule according to the first aspect of the present invention, for example an antibody or a CAR according to the first aspect of the present invention, the method comprising: expressing a nucleic acid molecule, or combination of multiple distinct nucleic acid molecules, according to the second aspect of the present invention, and/or a vector according to the third aspect of the present invention, in a cell, more particularly a recombinant cell, or a population of such cells (optionally a homogeneous or heterogeneous population of cells). Said cell or cells may be a cell according to the fifth aspect of the present invention, for example as described above.

Optionally, the method of the sixth aspect of the present invention may also comprise the step of isolating or purifying the thus-produced binding molecule from the cell; for example, wherein the binding molecule is an antibody according to the first aspect of the present invention, a functional fragment of said antibody, or an antibody of functional fragment thereof that comprises a fusion polypeptide sequence according to the first aspect of the present invention.

Accordingly, in certain embodiments, the binding molecules of the present disclosure may be purified. The term “purified,” as used herein, is intended to refer to a composition, isolatable from other components, wherein the binding molecules is purified to any degree relative to the state in which it is obtainable following initial production, for example in the state in which it is produced in a cell or cell culture. Where term “substantially purified” is used, this designation will refer to a composition in which the binding molecule forms the major component of a composition, such as constituting about 50%, about 60%, about 70%, about 80%, about 90%, about 95% or more (by weight) of the proteinaceous content in the composition.

Protein purification techniques are well known to those of skill in the art. These techniques involve, at one level, the crude fractionation of the cellular milieu to polypeptide and non-polypeptide fractions. Having separated a polypeptide of interest (e.g. a binding molecule of the present invention) from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody or other binding molecule of the present invention, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. For example, in the context of binding molecules according to the present invention which possess a binding specificity to ECD1 and/or ECD3 of US28, then suitable antigens may include one or more of peptide or polypeptide selected from either or both of the thirteenth and/or fourteenth aspects of the present invention, a combination of at least two (e.g. three) distinct peptides and/or polypeptides according to the fifteenth aspect of the present invention, a fusion protein according to the sixteenth aspect of the present invention, and/or a combination of at least two (e.g. three) distinct fusion proteins according to the seventeenth aspect of the present invention, a conjugate according to eighteenth aspect of the present invention, and/or a combination of at least two (e.g. three) distinct conjugates according the nineteenth aspect of the present invention.

Such methods often utilize the selection agent bound to a support, such as a column, filter or bead. The antibody or other binding molecule can be bound to a support, contaminants removed (e.g., washed away), and the antibody (or other binding molecule) released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptide within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

A seventh aspect of the present invention provides an isolated and/or purified binding molecule that is obtained, or obtainable, by the method of the sixth aspect of the present invention, optionally, wherein the isolated binding molecule is further formulated for administration to a subject.

In one embodiment binding molecules of the present invention can be formulated as a pharmaceutical composition; said composition comprising a pharmaceutically effective amount of one or more binding molecules of the present invention as defined herein. In a preferred embodiment, the pharmaceutical composition further comprises a pharmaceutically or veterinarially acceptable adjuvant, diluent or carrier, which will typically be selected with regard to the intended route of administration and standard pharmaceutical practice. The composition may be in the form of immediate-, delayed- or controlled-release applications. Preferably, the formulation is a unit dosage containing a daily dose or unit, daily sub-dose or an appropriate fraction thereof, of the active ingredient.

The phrases “pharmaceutical or veterinary acceptable” include reference to compositions that do not produce an adverse, allergic or other untoward reaction when administered to an animal or a human, as appropriate. The preparation of such pharmaceutical or veterinary compositions are known to those of skill in the art in the light of the present disclosure, as exemplified by Remington's Pharmaceutical Sciences, 18th Ed. Mack Printing Company, 1990, incorporated herein by reference. Moreover, for animal or human administration, it will be understood that preparations should meet sterility, pyrogenicity, general safety and purity standards as required by FDA Office of Biological Standards.

As used herein, “pharmaceutically or veterinarially acceptable carrier” includes any and all solvents, dispersion media, coatings, surfactants, antioxidants, preservatives (e.g., antibacterial agents, antifungal agents), isotonic agents, salts, preservatives, drugs, drug stabilizers, excipients, disintegration agents, such like materials and combinations thereof, as would be known to one of ordinary skill in medicine. Except insofar as any conventional carrier is incompatible with the active ingredient, its use in the therapeutic or pharmaceutical compositions is contemplated.

In one embodiment, the pharmaceutical composition further comprises a further drug, and/or other therapeutic, prophylactic or diagnostic agent.

In one embodiment, the binding molecules of the present invention as described herein, the composition described herein, or the pharmaceutical composition described herein, is formulated for oral, parenteral, intravenous, intra-arterial, intraperitoneal, intra-muscular, intra-ocular, intra-cranial, intra-cerebral, intra-osseous, intra-cerebroventricular, intra-thecal or subcutaneous administration.

Sterile injectable solutions may be prepared by incorporating the binding molecules of the present invention in the required amount in the appropriate solvent with various of the other ingredients enumerated above, as required, followed by sterilization. The pharmaceutical compositions may be best used in the form of a sterile aqueous solution which may contain other substances, for example, enough salts or glucose to make the solution isotonic with blood. The aqueous solutions may be suitably buffered (preferably to a pH of from 3 to 9), if necessary. The preparation of suitable pharmaceutical formulations under sterile conditions is readily accomplished by standard pharmaceutical techniques well-known to those skilled in medicine.

The pharmaceutical or veterinary compositions according to the invention may alternatively be formulated in the form of a powder, such as a sterile powder, which may be a lyophilised powder.

G. Combating HCMV and/or a Disease or Condition Associated with HCMV

An eleventh aspect of the present invention provides a method of combating HCMV or a disease or condition associated with HCMV.

In this context, the term “combatting” can optionally include any one or more of prophylaxis, vaccinating against, reducing the risk of, preventing, treating, ameliorating, slowing or preventing the progression of, reversing and/or curing, an HCMV infection or a disease or condition associated with an HCMV infection.

In one option, the term “combatting” can refer to therapeutic treatment. Such therapeutic treatment may result in a decrease in severity of disease symptoms, and/or an increase in frequency or duration of symptom-free periods. An amount adequate to accomplish this is defined as “therapeutically effective amount”.

In prophylactic applications, the subject of the method may not yet be exhibiting symptoms of HCMV infection or a disease or condition associated with an HCMV infection, and the method may be used to prevent or delay the development of symptoms. An amount adequate to accomplish this is defined as a “prophylactically effective amount”. The subject may have been identified as being at risk of developing the disease or condition by any suitable means.

The method of the eleventh aspect of the present invention comprises administering to a subject, or to ex vivo or in vitro cellular material, any one or more agents selected from the group consisting of:
i. a binding molecule according to the first aspect of the present invention,
ii. a functional fragment of said binding molecule according to the first aspect of the present invention,
iii. an isolated binding molecule according to the seventh aspect of the present invention,
iv. a nucleic acid molecule, or combination of multiple distinct nucleic acid molecules, according to the second aspect of the present invention,
v. a vector according to the third aspect of the present invention,
vi. a cell according to the fourth aspect of the present invention,
vii. a conjugate according to the eighth aspect of the present invention, and
viii. an isolated conjugate according to the tenth aspect of the present invention.

To put it another way, the eleventh aspect of the present invention provides one or more of said agents for use in combating a disease or condition associated with HCMV in a subject, or in ex vivo or in vitro cellular material.

Further, the eleventh aspect of the present invention provides for the use one or more of said agents in the manufacture of a medicament for combating a disease or condition associated with HCMV in a subject, or in ex vivo or in vitro cellular material.

The subject is preferably a human. However, it will also be understood that the subject can be non-human, such as any non-human mammal, for example a horse, dog, pig, cow, sheep, rat, mouse, guinea pig or a primate, for example wherein the non-human animal is a humanized animal model, having transplanted human cells that can be carriers of the HCMV.

The subject may optionally be a male, such as a male human.

The subject may optionally be a female, such as a female human.

The subject may, for example, be an adult, an adolescent, a juvenile, a child, an infant, a neonate, a foetus or an embryo; such as a human adult, human adolescent, human juvenile, human child, human infant, human neonate, human foetus or human embryo.

The subject may, for example, be a human infant aged less than 1, 2, 3, 4, 5, 6, 7 8, 9, 10, 11 or 12 months old.

The subject may be, for example, be a human (e.g. a male human and/or a female human) aged at least or more than 1 year old, for example, at least or greater than 2, 3, 4, 5, 6, 7, 8, 9, 10, 11, 12, 13, 14, 15, 16, 17, 18, 19, or 20 years old; and optionally less than 100, 90, 85, 80, 75, 65, 60, 55, 50, 45 or 40 years old.

The subject may be, for example, be a human (e.g. a male human and/or a female human) aged at least or more than 20 years old, for example, at least or greater than 25, 30, 35, 40, 45, 50, 55, 60, 65, 70, 75 or 80 years old; and optionally less than 100, 90 or 85 years old.

Said subject may be a subject with actual (including positively-diagnosed), suspected, or potential HCMV infection. A subject may, thus, be determined to have actual HCMV infection by direct detection of HCMV virus in a biological sample (e.g. blood, saliva and/or urine) taken from said subject.

Said subject may optionally be a subject that has received, is continuing to receive, or will subsequently receive any one or more other treatments for HCMV infection and/or any condition or symptom associated therewith. Two drugs known in the art that can be used to inhibit the progression of HCMV infection are ganciclovir and foscarnet. Typically, these are delivered intravenously, and typically treatment continues over a long period of time. A tablet form of ganciclovir, approved for CMV infections of the eye, has become available. Further, ganciclovir, delivered in the form of an intravitreal implant (e.g. Vitrasert Implant) is available as a means of providing long-term delivery of the drug to the eye. This delivery system typically requires a minor operation on the eye(s) in order to implant the delivery system. This form of delivery typically lasts for 3 to 6 months before it must be repeated. For babies with signs of congenital CMV infection at birth, antiviral medications, primarily valganciclovir, may improve hearing and developmental outcomes. Valganciclovir can have serious side effects and has only been studied in babies with signs of congenital CMV infection. HCMV immune globulin intravenous (human) (CMV IGIV) has also been used. CMV IGIV is an intravenous immune globulin enriched in antibodies against cytomegalovirus (CMV); it is approved by the U.S. FDA as a preventive measure (prophylaxis) against CMV disease associated with transplantation of the kidney, lung, liver, pancreas, and heart.

A subject having suspected HCMV infection may not necessarily have been directly tested for carrying HCMV virus, but may show one or more symptoms of HCMV infection. For example, symptoms associated with a HCMV infection (in particular, a lytic HMCV infection) in otherwise healthy adults can include symptoms similar to infectious mononucleosis, including for example any one, two, three or all four of: fatigue; fever; sore throat; and/or muscle aches. Additional symptoms, particularly in individuals with weakened immunity and/or immunocompromised individuals, may be, problems with any one, two, three, four, five, six, or all seven of: eyes, lungs, liver, oesophagus, stomach, intestines and/or brain. Symptoms associated with HCMV in infants with congenital HCMV infection (in particular lytic HMCV infection) may be, for example, any one, two, three, four, five, six, seven, eight or all nine of the following characteristics: premature birth; low birth weight; yellow skin and eyes (jaundice); enlarged and poorly functioning liver; purple skin splotches or a rash or both; abnormally small head (microencephaly); enlarged spleen; pneumonia; and/or seizures.

HCMV can be spread from an infected person to an uninfected person through numerous routes, including via body fluids, such as blood, saliva, urine, semen and breast milk. For example, transmission can occur by a subject touching their eyes or the inside of their nose or mouth after coming into contact with the body fluids of an infected person; by sexual contact with an infected person; by contact with (including the consumption of) breast milk from an infected mother; by contact with (e.g. implantation or transplantation) of biological material (organ, tissue, bone marrow or stem cell transplantation) or blood transfusions from an infected person; or during pregnancy and/or birth whereby an infected mother can pass the virus to her baby before or during birth. A subject with a potential HCMV infection may not have been directly tested for carrying HCMV virus and/or may not show one or more symptoms of HCMV infection, but may nevertheless have a history (e.g. for example within the preceding 70, 60, 50, 40, 30, 20, 10, 5, 4, 3, 2 or 1 years, the preceding 12, 11, 10, 9, 8, 7, 6, 5, 4, 3, 2 or 1 months, or a recent history such as within the preceding 4, 3, 2, or 1 weeks, or withing the preceding 7, 6, 5, 4, 3, 2 or 1 days) that puts them at risk of having contracted HCMV from one or more infected individuals, such as by any one or more of the routes defined above, and may therefore be characterised as a subject with a potential HCMV infection in accordance with the present invention.

The disease or condition associated with HCMV, to be combatted in accordance with the eleventh aspect of the present invention may be an HCMV infection or be associated with an HCMV infection. HMCV-infected cancers are a particular interest for the present invention, as discussed further below, although HCMV infections in any context, and any diseases or conditions associated with HCMV infection, are of interest to be combatted in accordance with the present invention.

Optionally, the disease, disorder or condition to be combatted in accordance with the eleventh aspect of the present invention may be associated with the expression of US28, including in particular the surface expression of US28 and the exposure of ECD1 (and optionally ECD3) of US28 at the surface of a cell. This may include, for example, any disease or condition associated with cells which express US28. For example, the cells may be unwanted cells. The unwanted cells may be any cell whose presence in a host is undesired. Accordingly, the disease or condition associated with expression of US28 may be any condition characterised by the presence of cells that express US28 and which are unwanted, for example, any biological or medical condition or disorder in which at least part of the pathology is mediated by the presence of such unwanted cells expressing US28. The condition may be caused by the presence of the unwanted cells or else the presence of the unwanted cells may be an effect of the condition.

By expression of US28 we include the meaning that US28 protein is able to be detected on, or in, a cell, or in extracts prepared from cells, or expression of the polypeptide may be inferred by detection of US28-encoding mRNA. In order to confirm the expression of US28 on, or in, a cell, a variety of assays may be performed. Such assays include, for example, biochemical assays well known to those skilled in the art, such as detecting the presence of a particular protein (i.e. US28) by immunological means (ELISAs and Western blots), or molecular biological assays well known to those of skill in the art, such as Northern blotting, RT-PCR and PCR for detecting the presence US28 mRNA. However, as described further herein, the detection of US28 can be complicated by variation at the amino acid level between different strains of HCMV and/or by polymorphisms at the genetic level, and certain methods can be sensitive to and/or allow the detection of US28 protein expression from a limited number of strains of HCMV. This can lead to a failure to detect HCMV infections from other strains. It may be preferred that US28 expression is determined using a strain-agnostic approach. Binding molecules of the present invention can provide strain agnostic binding to ECD1 and/or ECD3 of US28, and may be a particularly preferred approach to assessing US28 expression on, or in, one or more cells, or in extracts prepared from cells, of a subject.

Optionally, an HCMV infection may be asymptomatic, and/or it may be undiagnosed. Optionally, an HCMV infection may be a latent infection, for example it may be characterized by low-level or non-existent virus replication with the viral genome residing predominantly in the CD34⁺ hematopoietic progenitor cell population residing in the bone marrow.

The subject in whom the disease or condition associated with HCMV is to be combatted may be an immunocompromised patient, for example a patient in whom primary HCMV infection, re-infection or reactivation can cause a disease, such as life-threatening disease, that affects one or many organs. An immunocompromised person may, for example be characterised by having one or more serious problems that affect any one, two, three, four, five, six or all seven of their: eyes, lungs, liver, esophagus, stomach, intestines and/or brain. An immunocompromised subject may, for example, be a person that has, has had, or is intended to be the recipient of, a transplant of biological material, such as an organ, stem cell or bone marrow transplant; in addition, or alternatively, in one option, the biological material to be transplanted may be ex vivo or in vitro cellular material that is to be treated in accordance with the eleventh aspect of the present invention.

HCMV is one of the most common congenital viral infections and most important cause of birth defects. Infants can also become infected during birth or shortly afterward (perinatal HCMV); this group includes babies infected through breast milk. Accordingly, condition associated with HCMV may be a congenital HCMV infection or a congenital HCMV-associated birth defect or may be a perinatal HCMV infection or a perinatal HCMV-associated disease or condition. Congenital HCMV infections in babies may, for example, be characterised by conditions that display any one, two, three, four, five, six, seven, eight or all nine of the following characteristics: premature birth; low birth weight; yellow skin and eyes (jaundice); enlarged and poorly functioning liver; purple skin splotches or a rash or both; abnormally small head (microencephaly); enlarged spleen; pneumonia; and/or seizures; in any event, the direct detection of HCMV infection in said babies can be used to confirm congenital infection.

The subject may, for example, be an infant. Said infant may be a neonate born of a mother with actual (including positively-diagnosed), suspected, or potential HCMV infection. Said infant may be an infant having a parent (in particular, a mother) with actual (including positively-diagnosed), suspected, or potential HCMV infection. Said infant may be an infant that is breast-fed by a mother with actual (including positively-diagnosed), suspected, or potential HCMV infection. The subject may be a foetus or embryo carried by a mother that has actual (including positively-diagnosed), suspected, or potential HCMV infection. The subject may be a mother, a pregnant female, or a prospective mother that has actual (including positively-diagnosed), suspected, or potential HCMV infection. The subject may be a breast-feeding mother that has actual (including positively-diagnosed), suspected, or potential HCMV infection. The subject may be a female having actual (including positively-diagnosed), suspected, or potential HCMV infection, prior to conception.

A condition associated with HCMV may be, for example, a cardiovascular disease such as atherosclerosis, or an autoimmune disease.

HCMV serostatus may additionally impact the clinical course of burns, trauma, sepsis, and infection with pathogens such as bacteria and/or viruses other than HCMV; and a subject in whom the disease or condition associated with HCMV is to be combatted may be a subject that is suffering from burn damage, trauma, sepsis and/or a pathogenic infection (e.g. bacterial infection or infection with a virus other than HCMV) and/or said disease or condition associated with HCMV may include any one or more of burn damage, trauma, sepsis and/or a pathogenic infection (e.g. bacterial infection or infection with a virus other than HCMV). A condition associated with HCMV, particularly in individuals with weakened immunity and/or immunocompromised individuals, may be, for example, any one or more of: vision loss, due to inflammation of the light-sensing layer of the eye (retinitis); digestive system problems, including inflammation of the colon (colitis), oesophagus (esophagitis) and/or liver (hepatitis); nervous system problems, including brain inflammation (encephalitis); and/or pneumonia.

A condition associated with HCMV in infants with congenital HCMV infection may be, for example, any one or more of: hearing loss, intellectual disability, vision problems, seizures, lack of coordination, weakness and/or problems using muscles.

The HCMV infection may, in one embodiment, be a single strain infection.

In an alternative embodiment, the HCMV infection comprises a multi-strain HCMV infection, wherein the multi-strain HCMV infection comprises infection with more than one different strain of HCMV, for example two or more different HCMV strains. Such multi-strain infections are common, and it will be appreciated that an agent that is able to target only some, but not all, of the HCMV strains in a multi-strain infection, may be at risk of creating a selective pressure that can promote the non-targeted strain(s) in the multi-strain HCMV infection, by inhibiting or removing one or more competing HCMV strains that are targeted by said agent. Accordingly, in the case of selecting an approach to combat actual, suspected, or potential multi-strain HCMV infections, it is particularly preferred to use a treatment modality that provides a strain agnostic effect. The use of a strain agnostic agent, in accordance with the present invention, can thereby be used to prevent the creation of conditions in a treated subject that selectively promote the development of infection by one or more HCMV strains present in a multi-strain HCMV infection, because all HCMV strains in the multi-strain HCMV infection are targeted by the strain agnostic agent.

Optionally, the two or more different HCMV strains of a multi-strain infection encode different US28 protein encodes sequences. Said two or more strains may encode US28 proteins that differ in one or more of the extracellular regions, such as in the N-terminal (ECD1) regional, the first extracellular loop (ECD2) region, the second extracellular loop (ECD3) region, and/or the third extracellular loop (ECD4) region. In one embodiment of interest, the two or more HCMV strains in a multi-strain HCMV infection each encode a US28 protein that differs from the other at least in one or more positions of the N-terminal (ECD1) region; for example they may differ at 1, 2, 3, 4, 5, 6, 8, 9, 10 or more amino acid positions in the N-terminal (ECD1) region. The differences in one or more positions of the N-terminal (ECD1) region may be outside of positions 26-37; for example the one or more positions that differ may be within positions 1-25 (such as any one or more of positions 8, 15, 16, 18, 19, 24, 25, including but not limited to any one or more of the sequence variations listed in Table 3 of the present application), for example within one or more of positions 1-10, 16, 18 and/or 19, wherein the positions are with respect to the numbering of positions in SEQ ID NO: 5. Additionally, or alternatively, in another embodiment of interest, the two or more HCMV strains in a multi-strain HCMV infection each encode a US28 protein that differs from the other at one or more positions of the second extracellular loop (ECD3) region, for example one or more of the HCMV strains in a multi-strain HCMV infection may encode a US28 protein that encodes the 4N-variant of ECD3, and one or more of the other HCMV strains in a multi-strain HCMV infection may encode a US28 protein that encodes the 4D-variant of ECD3. For example, the two or more HCMV strains that encode different US28 protein encodes sequences may be selected from any two or more of HCMV strain DB (Accession number KT959235), HCMV strain Toledo (Accession number GU937742), HCMV strain Towne (Accession number FJ616285), HCMV strain VR1814 (Accession number GU179289), HCMV strain TB40/E (Accession number KF297339), HCMV strain Merlin (Accession number AY446894), HCMV strain JP (Accession number GQ221975), HCMV strain Ad169 (Accession number X17403.1), HCMV strain AF1 (Accession number GU179291.1), HCMV strain VHL/E (Accession number L20501.1), HCMV strain BL (Accession number MW980585), HCMV strain DAVIS (Accession number JX512198.1). HCMV strain TR (Accession number KF021605.1).

The disease or condition associated with HCMV, to be combatted in accordance with the eleventh aspect of the present invention may be a latent HCMV infection (for example, a single or multi-strain latent HCMV infection) or be associated with a latent HCMV infection (optionally a multi-strain latent HCMV infection).

The disease or condition associated with HCMV, to be combatted in accordance with the eleventh aspect of the present invention may be a lytic HCMV infection (optionally a multi-strain lytic HCMV infection) or be associated with a lytic HCMV infection (optionally a multi-strain lytic HCMV infection).

The disease or condition associated with HCMV, to be combatted in accordance with the eleventh aspect of the present invention may be a congenital HCMV infection (for example, a single or multi-strain infection), such as a latent congenital single or multi-strain HCMV infection or a lytic congenital single or multi-strain HCMV infection;

The disease or condition associated with HCMV, to be combatted in accordance with the eleventh aspect of the present invention may be cancer, for example HCMV-infected cancer (optionally a single-strain, or multi-strain, HCMV infected cancer), such as latent HCMV-infected cancer (optionally a single-strain, or multi-strain, latent HCMV infected cancer).

The cancer may optionally be at the site of a primary, secondary or any other tumour in the body of a subject.

Tumors are often assigned a grade and a stage. The stage of a solid tumor refers to its size or extent and whether or not it has spread to other organs and tissues. The grade of a tumor—the cancer grade—is an indication of how quickly it is likely to grow and spread.

For example, the cancer may be stage 0, stage I, stage II, stage III or stage IV, or a subdivision of any one or more thereof. The characteristics of these different stages and the subdivisions thereof are well known in the art. However, in general terms: Stage 0 indicates that the cancer is where it started (in situ) and has not spread. Stage I indicates that the cancer is small and has not spread anywhere else. Stage II indicates that the cancer has grown, but has not spread. Stage III indicates that the cancer is larger and may have spread to the surrounding tissues and/or the lymph nodes (part of the lymphatic system). Stage IV indicates that the cancer has spread from where it started to at least one other body organ; also known as “secondary” or “metastatic” cancer.

In one embodiment, the cancer may be a stage of cancer classified by the TNM Staging System. This is system that was developed and is maintained by the AJCC and the Union for International Cancer Control (UICC). It is the most commonly used staging system by medical professionals around the world. The TNM classification system was developed as a tool for doctors to stage different types of cancer based on certain, standardized criteria. The TNM Staging System is based on the extent of the tumor (T), the extent of spread to the lymph nodes (N), and the presence of metastasis (M).
The T category describes the original (primary) tumor. TX refers to a primary tumor that cannot be evaluated. TO refers to no evidence of a primary tumor. Tis refers to a carcinoma in situ (early cancer that has not spread to neighbouring tissue). T1, T2, T3 and T4 relate to the size and/or extent of the primary tumor.
The N category describes whether or not the cancer has reached nearby lymph nodes. NX indicates that regional lymph nodes cannot be evaluated. NO indicates no regional lymph node involvement (no cancer found in the lymph nodes). N1, N2 and N3 indicate involvement of regional lymph nodes (number and/or extent of spread).
The M category tells whether there are distant metastases (spread of cancer to other parts of the body). M0 indicates no distant metastasis (cancer has not spread to other parts of the body). M1 indicates distant metastasis (cancer has spread to distant parts of the body).

Because each cancer type has its own classification system, letters and numbers do not always mean the same thing for every kind of cancer. Once the T, N, and M are determined, they are combined, and an overall stage of 0, I, II, III, IV can be assigned. Sometimes these stages are subdivided as well, using letters suther guidance can be found at https://cancerstaging.org.

In some cancer types, non-anatomic factors can be taken into account for assigning the anatomic stage/prognostic group. These are clearly defined in each chapter of the AJCC Cancer Staging Manual (e.g. Gleason Score in Prostate). These factors are collected separately from T, N, and M, which remain purely anatomic and are used to assign stage groups. Where non-anatomic factors are used in groupings, there is a definition of the groupings provided for cases where the non-anatomic factor is not available (X) or where it is desired to assign a group ignoring the non-anatomic factor.

Stage I cancers are the least advanced and often have a better prognosis. Higher stage cancers are often more advanced but, in many cases, can still be treated successfully.

In an additional and/or alternative option, the cancer may be of a specified grade. Grading is typically based on the differentiation of cells (degree of resemblance to normal cells). The specified grade of the cancer may, for example, be grade I, grade II, grade III or grade IV cancer, or a combination of two or three categories. The characteristics of these different grades are well known in the art. However, in general terms: A grade I cancer is a type of cancer in which the cells that resemble normal cells and are not growing rapidly; a grade II cancer is a type of cancer in which the cancer cells do not look like normal cells and are growing faster than normal cells; and grades III and IV are a type of cancer in which cancer cells look abnormal and may grow or spread more aggressively. Growth characteristics may, for example, be assessed in some cancers based on frequency of dividing cells.

The cancer may, for example, be a type of cancer that is selected from the group consisting of carcinoma, sarcoma, myeloma, leukemia, lymphoma and a mixed type of cancer.

Carcinoma refers to a malignant neoplasm of epithelial origin or cancer of the internal or external lining of the body. Carcinomas, malignancies of epithelial tissue, account for 80 to 90 percent of all cancer cases. Epithelial tissue is found throughout the body. It is present in the skin, as well as the covering and lining of organs and internal passageways, such as the gastrointestinal tract, as further discussed above.

Carcinomas may be divided into two major subtypes: adenocarcinoma, which develops in a glandular organ, and squamous cell carcinoma, which originates in the squamous epithelium.

Adenocarcinomas generally occur in mucus membranes and are first seen as a thickened plaque-like white mucosa. They often spread easily through the soft tissue where they occur. Squamous cell carcinomas occur in many areas of the body.

Most carcinomas affect organs or glands capable of secretion, such as the breasts, which produce milk, or the lungs, which secrete mucus, or colon or prostate or bladder.

In one embodiment, the carcinoma may be a cancer that arises from epithelial cells that is selected from breast cancer, basal cell carcinoma, adenocarcinoma, gastrointestinal cancer, lip cancer, mouth cancer, oesophageal cancer, small bowel cancer and stomach cancer, colon cancer, liver cancer, bladder cancer, pancreas cancer, ovary cancer, cervical cancer, lung cancer, and skin cancer, such as squamous cell and basal cell cancers, prostate cancer, renal cell carcinoma, and other known cancers that effect epithelial cells throughout the body.

Sarcoma refers to cancer that originates in supportive and connective tissues such as bones, tendons, cartilage, muscle, and fat. Generally occurring in young adults, the most common sarcoma often develops as a painful mass on the bone. Sarcomas usually resemble the tissue in which they grow.

Examples of sarcomas are: osteosarcoma or osteogenic sarcoma (bone), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or haemangioendothelioma (blood vessels), liposarcoma (adipose tissue), glioma or astrocytoma (neurogenic connective tissue found in the brain), myxosarcoma (primitive embryonic connective tissue), mesenchymous or mixed mesodermal tumour (mixed connective tissue types).

Myeloma is cancer that originates in the plasma cells of bone marrow. The plasma cells produce some of the proteins found in blood.

The cancer may be a solid cancer, or a liquid cancer.

For example, leukemias (“liquid cancers” or “blood cancers”) are cancers of the bone marrow (the site of blood cell production). The disease is often associated with the overproduction of immature white blood cells. These immature white blood cells do not perform as well as they should, therefore the patient is often prone to infection. Leukemia also affects red blood cells and can cause poor blood clotting and fatigue due to anemia. Examples of leukemia include:
Myelogenous or granulocytic leukemia (malignancy of the myeloid and granulocytic white blood cell series)
Lymphatic, lymphocytic, or lymphoblastic leukemia (malignancy of the lymphoid and lymphocytic blood cell series)
Polycythaemia Vera or erythraemia (malignancy of various blood cell products, but with red cells predominating)

Lymphomas develop in the glands or nodes of the lymphatic system, a network of vessels, nodes, and organs (specifically the spleen, tonsils, and thymus) that purify bodily fluids and produce infection-fighting white blood cells, or lymphocytes. Unlike the leukemias which are sometimes called “liquid cancers,” lymphomas are “solid cancers.” Lymphomas may also occur in specific organs such as the stomach, breast or brain. These lymphomas are referred to as extra nodal lymphomas. The lymphomas are subclassified into two categories: Hodgkin lymphoma and Non-Hodgkin lymphoma. The presence of Reed-Sternberg cells in Hodgkin lymphoma diagnostically distinguishes Hodgkin lymphoma from Non-Hodgkin lymphoma.

Mixed types of cancer may include cancers in which the type components are within one category or from different categories of cancer. Some examples are: adenosquamous carcinoma; mixed mesodermal tumour; carcinosarcoma; and teratocarcinoma.

Optionally, the cancer may be a primary cancer, or a metastatic cancer. A primary cancer refers to cancer cells in a primary tumour, which is a tumour appearing at a first site within the subject and which can be distinguished from a metastatic tumour which appears in the body of the subject at a remote site from the primary tumour. A metastatic cancer results from metastasis, which refers to the condition of spread of cancer from the organ of origin to additional distal sites in the patient.

For example, the type of cancer may optionally be selected any one or more of the following list:
Acute Lymphoblastic Leukemia (ALL),
Acute Myeloid Leukemia (AML),
Cancer in Adolescents (e.g. in an adolescent between the age of 12-18 years),
Adrenocortical Carcinoma, also including for example:
   Childhood Adrenocortical Carcinoma
AIDS-Related Cancers, also including for example:
   Kaposi Sarcoma (Soft Tissue Sarcoma)
   AIDS-Related Lymphoma (Lymphoma)
   Primary CNS Lymphoma (Lymphoma)
Anal Cancer
Appendix Cancer
Childhood Astrocytomas (Brain Cancer)
Atypical Teratoid/Rhabdoid Tumour, Childhood, Central Nervous System (Brain Cancer)
Basal Cell Carcinoma of the Skin
Bile Duct Cancer
Bladder Cancer, also including for example:
   Childhood Bladder Cancer
Bone Cancer (for example, Ewing Sarcoma, Osteosarcoma or Malignant Fibrous Histiocytoma)
Brain Tumours (including, for example, glioma or glioblastoma)
Breast Cancer, also including for example:
   Childhood Breast Cancer
Childhood Bronchial Tumours
Burkitt Lymphoma
Carcinoid Tumour (Gastrointestinal), also including for example:
   Childhood Carcinoid Tumours
Carcinoma of Unknown Primary, also including for example:
   Childhood Carcinoma of Unknown Primary
Childhood Cardiac (Heart) Tumours
Central Nervous System, also including for example:
   Childhood Atypical Teratoid/Rhabdoid Tumour, (Brain Cancer)
   Childhood Embryonal Tumours, (Brain Cancer)
   Childhood Germ Cell Tumour, (Brain Cancer)
   Primary CNS Lymphoma
Cervical Cancer, also including for example:
   Childhood Cervical Cancer
Childhood Cancers (e.g. a child that is under the age of 18 years old, preferably under the age of 16, 14 or 1.2 years old, such as within the range of 1-12 years),
Unusual Cancers of Childhood,
Cholangiocarcinoma
Childhood Chordoma,
Chronic Lymphocytic Leukemia (CLL)
Chronic Myelogenous Leukemia (CML)
Chronic Myeloproliferative Neoplasms
Colorectal Cancer, also including for example:
   Childhood Colorectal Cancer
Childhood Craniopharyngioms (Brain Cancer)
Cutaneous T-Cell Lymphoma
Ductal Carcinoma in situ (DCIS)
Embryonal Tumours, Central Nervous System, Childhood (Brain Cancer)
Endometrial Cancer (Uterine Cancer)
Childhood Ependymoma (Brain Cancer)
Oesophageal Cancer, also including for example:
   Childhood Oesophageal Cancer
Esthesioneuroblastoma (Head and Neck Cancer)
Ewing Sarcoma (Bone Cancer)
Childhood Extracranial Germ Cell Tumour,
Extragonadal Germ Cell Tumour
Eye Cancer, also including for example:
   Childhood Intraocular Melanoma
   Intraocular Melanoma
   Retinoblastoma
Fallopian Tube Cancer
Fibrous Histiocytoma of Bone, Malignant, and Osteosarcoma
Gallbladder Cancer
Gastric (Stomach) Cancer, also including for example:
   Childhood Gastric (Stomach) Cancer
Gastrointestinal Carcinoid Tumour
Gastrointestinal Stromal Tumours (GIST) (Soft Tissue Sarcoma), also including for example:
   Childhood Gastrointestinal Stromal Tumours
Germ Cell Tumours, also including for example:
   Childhood Central Nervous System Germ Cell Tumours (Brain Cancer)
   Childhood Extracranial Germ Cell Tumours
   Extragonadal Germ Cell Tumours
   Ovarian Germ Cell Tumours
   Testicular Cancer
Gestational Trophoblastic Disease
Hairy Cell Leukemia
Head and Neck Cancer
Childhood Heart Tumours
Hepatocellular (Liver) Cancer
Histiocytosis, Langerhans Cell
Hodgkin Lymphoma
Hypopharyngeal Cancer (Head and Neck Cancer)
Intraocular Melanoma, also including for example:
   Childhood Intraocular Melanoma
Islet Cell Tumours, Pancreatic Neuroendocrine Tumours
Kaposi Sarcoma (Soft Tissue Sarcoma)
Kidney (Renal Cell) Cancer
Langerhans Cell Histiocytosis
Laryngeal Cancer (Head and Neck Cancer)
Leukemia
Lip and Oral Cavity Cancer (Head and Neck Cancer)
Liver Cancer
Lung Cancer (Non-Small Cell and Small Cell), also including for example:
   Childhood Lung Cancer
Lymphoma
Male Breast Cancer
Malignant Fibrous Histiocytoma of Bone and Osteosarcoma
Melanoma, also including for example:
   Childhood Melanoma
Intraocular (Eye) Melanoma, also including for example:
   Childhood Intraocular Melanoma
Merkel Cell Carcinoma (Skin Cancer)
Malignant Mesothelioma, also including for example:
   Childhood Mesothelioma
Metastatic Cancer
M Metastatic Squamous Neck Cancer with Occult Primary (Head and Neck Cancer)
Midline Tract Carcinoma with NUT Gene Changes
Mouth Cancer (Head and Neck Cancer)
Multiple Endocrine Neoplasia Syndromes
Multiple Myeloma/Plasma Cell Neoplasms
Mycosis Fungoides (Lymphoma)
Myelodysplastic Syndromes, Myelodysplastic/Myeloproliferative Neoplasm
Chronic Myelogenous Leukemia (CML)
Acute Myeloid Leukemia (AML)
Chronic Myeloproliferative Neoplasms
Nasal Cavity and Paranasal Sinus Cancer (Head and Neck Cancer)
Nasopharyngeal Cancer (Head and Neck Cancer)
m Neuroblastoma
Non-Hodgkin Lymphoma
Non-Small Cell Lung Cancer
Oral Cancer, Lip and Oral Cavity Cancer and Oropharyngeal Cancer (Head and Neck Cancer)
Osteosarcoma and Malignant Fibrous Histiocytoma of Bone
Ovarian Cancer, also including for example:
   Childhood Ovarian Cancer
Pancreatic Cancer, also including for example:
   Childhood Pancreatic Cancer
Pancreatic Neuroendocrine Tumours (Islet Cell Tumours)
Papillomatosis (Childhood Laryngeal)
Paraganglioma, also including for example:
   Childhood Paraganglioma
Paranasal Sinus and Nasal Cavity Cancer (Head and Neck Cancer)
Parathyroid Cancer
Penile Cancer
Pharyngeal Cancer (Head and Neck Cancer)
Pheochromocytoma, also including for example:
   Childhood Pheochromocytoma
Pituitary Tumour
Plasma Cell Neoplasm/Multiple Myeloma
Pleuropulmonary Blastoma
Pregnancy and Breast Cancer (i.e. breast cancer in a pregnant female)
Primary Central Nervous System (CNS) Lymphoma
Primary Peritoneal Cancer
Prostate Cancer
Rectal Cancer
Recurrent Cancer
Renal Cell (Kidney) Cancer
Retinoblastoma
Childhood Rhabdomyosarcoma (Soft Tissue Sarcoma)
Salivary Gland Cancer (Head and Neck Cancer)
Sarcoma, also including for example:
   Childhood Rhabdomyosarcoma (Soft Tissue Sarcoma)
   Childhood Vascular Tumours (Soft Tissue Sarcoma)
   Ewing Sarcoma (Bone Cancer)
   Kaposi Sarcoma (Soft Tissue Sarcoma)
   Osteosarcoma (Bone Cancer)
   Soft Tissue Sarcoma
   Uterine Sarcoma
Sézary Syndrome (Lymphoma)
Skin Cancer, also including for example:
   Childhood Skin Cancer
Small Cell Lung Cancer
Small Intestine Cancer
Soft Tissue Sarcoma
m Squamous Cell Carcinoma of the Skin
Metastatic Squamous Neck Cancer with Occult Primary (Head and Neck Cancer)
Stomach (Gastric) Cancer, also including for example:
   Childhood Stomach (Gastric) Cancer
Cutaneous T-Cell Lymphoma
Testicular Cancer, also including for example:
   Childhood Testicular Cancer
Throat Cancer (Head and Neck Cancer), also including for example:
   Nasopharyngeal Cancer
   Oropharyngeal Cancer
   Hypopharyngeal Cancer
Thymoma and Thymic Carcinoma
Thyroid Cancer
Transitional Cell Cancer of the Renal Pelvis and Ureter (Kidney (Renal Cell) Cancer)
m Carcinoma of Unknown Primary, also including for example:
   Childhood Cancer of Unknown Primary
Unusual Cancers of Childhood
Ureter and Renal Pelvis, Transitional Cell Cancer (Kidney (Renal Cell) Cancer
Urethral Cancer
Endometrial Uterine Cancer
Uterine Sarcoma
Vaginal Cancer, also including for example:
   a Childhood Vaginal Cancer
Vascular Tumours (Soft Tissue Sarcoma)
Vulvar Cancer
Wilms Tumour and Other Childhood Kidney Tumours
Cancer in Young Adults (e.g. in a young adult ages 16-30, such as 18-30 years old; optionally less than 28, 26, 24, 22 or 20 years old)

More specifically, the type of cancer may optionally be selected any one or more of the following list:

Skin Cancer: There are three primary types of skin cancer: basal cell, squamous cell, and melanoma. These cancers are derived from the epidermal layers with the same names. Melanomas are derived from the melanocytes, or pigment cells, in the deepest level of the epidermis. Basal cell and squamous cell cancers usually occur on parts of the body exposed to the sun, such as the face, ears, and extremities.

Lung Cancer: Lung cancer is very difficult to detect at an early stage because the symptoms often do not appear until the disease has advanced. The symptoms include persistent cough, sputum streaked with blood, chest pain, and repeated attacks of pneumonia or bronchitis.

Female or Male Breast Cancer: It has been estimated that in the U.S., about 1 in 8 women will eventually develop breast cancer in her lifetime. Most breast cancers are ductal carcinomas. Women most likely to develop the disease are those over the age of 50; those who have already had cancer in one breast; those whose mother or sister had breast cancer; those who never had children; and those who had their first child after the age of 30. Other risk factors include obesity, a high-fat diet, early menarche (age menstruation begins) and late menopause (age menstruation ceases).

Prostate Cancer: Cancer of the prostate is found mainly in older men. As men age, the prostate may enlarge and block the urethra or bladder. This may cause difficulty in urination or interfere with sexual functions. This condition is called benign prostatic hypertrophy (BPH). Although BPH is not cancerous, surgery may be needed to correct it. The symptoms of BPH, or of other problems in the prostate, may be similar to symptoms for prostate cancer.

Colon and/or Rectum Cancer: Colorectal cancer (CRC) is a disease typically originating from the epithelial cells lining the colon or rectum of the gastrointestinal tract. Of the cancers that affect the large intestine, about 70 percent occur in the colon and about 30 percent in the rectum. These cancers are the third most common cancers overall. Symptoms include blood in the stool, which can be tested for by a simple faecal occult blood test, or a change in bowel habits, such as severe constipation or diarrhoea.

Uterus (Corpus Uteri) Cancer: The uterus is the sac in a woman's pelvis which allows a baby to develop from a fertilized egg and protects it until birth. Cancer of the uterus is the most common gynaecologic malignancy. This cancer occurs infrequently in women under 40 years of age. It occurs most frequently after the age of 60. The presenting symptom is usually abnormal uterine bleeding. An endometrial biopsy or D&C is often performed to confirm the diagnosis.

In addition to cancer types named after the primary site discussed above, there are many other examples such as brain cancer, testicular cancer, bladder cancer, and so on.

The disease or condition associated with HCMV, to be combatted in accordance with the eleventh aspect of the present invention may, in particular, be an epithelial cancer; optionally wherein the epithelial cancer is breast cancer; for example, wherein the breast cancer is triple negative breast cancer (TNBC), or a HER2-positive breast cancer (as described herein). Said forms of epithelial cancer may optionally be a single-strain, or multi-strain, form of HCMV infected epithelial cancer, for example a latent HCMV-infected form of epithelial cancer (optionally a single-strain, or multi-strain, latent HCMV infected cancer).

The disease or condition associated with HCMV, to be combatted in accordance with the eleventh aspect of the present invention may be a metastasising and/or aggressive form of cancer. Said forms of metastasising and/or aggressive cancer may optionally be a single-strain, or multi-strain, form of HCMV infected metastasising and/or aggressive cancer, for example a latent HCMV-infected form of metastasising and/or aggressive cancer (optionally a single-strain, or multi-strain, latent HCMV infected cancer).

In some embodiments, the disease or condition associated with HCMV, to be combatted in accordance with the eleventh aspect of the present invention may be glioblastoma. In other embodiment described herein, the disease or condition is not glioblastoma and/or the subject to be treated does not have and/or has not been diagnosed has having glioblastoma.

In some embodiments, the subject (or the ex vivo or in vitro cellular material) to be treated in accordance with the eleventh aspect of the present invention may have, and/or have been diagnosed has having or possessing, HCMV-infected cancer cells, such as latent HCMV-infected cancer cells.

In some embodiments, the subject to be treated in accordance with the eleventh aspect of the present invention may be, or intended to be, the recipient of a cellular material, such as the donation of a cellular product. Said cellular product may, for example, comprise, consist essentially of, or consist of, living ex vivo cellular material selected from the group that includes: one or more types of ex vivo cells; one or more types of ex vivo cell cultures; one or more types of ex vivo tissues; one or more types of ex vivo tissue cultures; one or more types of ex vivo organs; and/or one or more types of ex vivo organ cultures. Optionally, the cellular product may be derived, directly or indirectly, from a living donor.

In some embodiments, the subject to be treated in accordance with the eleventh aspect of the present invention may be, or intended to be the donor of a cellular material, such as the donor of a cellular product. Said cellular product may be comprise, consist essentially of, or consist of any one or more of cells, tissue or an organ from said donor.

In some embodiments, the ex vivo or in vitro cellular material to be treated in accordance with the eleventh aspect of the present invention may be an ex vivo cellular product. Said ex vivo cellular product may, for example, comprise, consist essentially of, or consist of, living ex vivo cellular material selected from the group that includes: one or more types of ex vivo cells; one or more types of ex vivo cell cultures; one or more types of ex vivo tissues; one or more types of ex vivo tissue cultures; one or more types of ex vivo organs; and/or one or more types of ex vivo organ cultures. Optionally, the cellular product may be derived, directly or indirectly, from a living donor.

In one embodiment, the one or more agents to be used in accordance with the eleventh aspect of the present invention may, for example, be (or include one or more agents) selected from the group consisting of:
i. a therapeutic antibody as defined by the first aspect of the present invention,
ii. a functional fragment of said therapeutic antibody as defined by the first aspect of the present invention,
iii. a therapeutic antibody that comprises as fusion polypeptide sequence as defined by the first aspect of the present invention; and
iv. a functional fragment of said therapeutic antibody that comprises as fusion polypeptide sequence as defined by the first aspect of the present invention.

For example, the one or more agents used in accordance with the eleventh aspect of the present invention may be (or include one or more agents selected from) a bispecific antibody as defined by the first aspect of the present invention. Without limitation, this may be a bispecific immune cell engager antibody. An exemplary embodiment thereof is a bispecific T-cell engager (BiTE) antibody, optionally wherein, in addition to the region that comprises the binding molecule of the first aspect of the present invention which has binding specificity for the ECD1 region of the US28 protein, the BiTE antibody further comprises a T-cell engaging domain, such as a CD3-binding domain.

In a further embodiment, the one or more agents to be used in accordance with the eleventh aspect of the present invention may, for example, be (or include) a conjugate according to the eighth aspect of the present invention and/or the tenth aspect of the present invention, such as conjugate that is an antibody-drug conjugate (“ADC”), or a conjugate that comprises radioactive moiety, such as conjugate that is suitable for use in radioimmunotherapy (“RIT”).

In another embodiment, the one or more agents to be used in accordance with the eleventh aspect of the present invention may, for example, be (or include) a cell (or population of cells, for example a homogeneous population of cells) wherein the or each cell comprises a CAR according to the fourth aspect of the present invention. Said cell or population of cells is typically isolated and/or formulated for administration to a subject.

### H. Combination Treatments:

Optionally, in accordance with the eleventh aspect of the present invention, the subject may be administered a further substance, such as a further therapeutic, prophylactic, diagnostic, prognostic, or theragnostic substance, and optionally wherein the further substance may be administered separately, sequentially or simultaneously with the, or each of the one or more agents.

Accordingly, the eleventh aspect of the present invention also provides a method of treating a subject in need thereof, by administering to the subject a therapeutic, prophylactic, diagnostic, prognostic, or theragnostic substance, wherein the subject is also treated separately, sequentially (for example, before, or after), or simultaneously, with the, or each of the one or more agents.

In the embodiment in which the treatment is simultaneous, then the one or more agents to be used in accordance with the eleventh aspect of the present invention may be formulated and/or administered in combination with the therapeutic, prophylactic, diagnostic, prognostic, or theragnostic substance; or may be formulated separately but administered simultaneously as two separate formulations.

For example, in one embodiment, the disease or condition to be treated may be a form of cancer (such as one or more forms of cancer as disclosed above), and the additional therapeutic, prophylactic, diagnostic, prognostic, or theragnostic substance may be targeted to the cancer.

Thus, in the context of the present disclosure, it also is contemplated that binding molecule of the present invention and other therapeutic agents as defined by the eleventh aspect of the present invention could be used similarly in the treatment of cancer in conjunction with chemo- or radiotherapeutic intervention, or other treatments.

Said different forms of treatment may precede or follow the other by intervals ranging from minutes to weeks. For example, within about 12-24 hours of each other and, more preferably, within about 6-12 hours of each other, with a delay time of only about 12 hours being one preferred option. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Administration of the therapeutic agents of the present invention to a patient will follow general protocols for the administration of that particular secondary therapy, taking into account the toxicity, if any, of the treatment. It is expected that the treatment cycles would be repeated as necessary. It also is contemplated that various standard therapies, as well as surgical intervention, may be applied in combination with the described cancer therapies.

The skilled artisan ^{is} directed to “Remington's Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments, the combination therapy comprises administration of a first binding molecule or other therapeutic agent of the present invention with specificity for a polypeptide consisting of the amino acid sequence of TDVLNQSKPVTL (SEQ ID NO: 177) within ECD1 of a US28 protein of HCMV, and a second binding molecule or other therapeutic with specificity for ECD3, for example as defined above with respect to ECD3-binding of the the binding molecules of the present invention but without the anti-SEQ 177 binding activity. Optionally, in the case of such combination treatment, the first binding molecule or other therapeutic agent of the present invention does not comprise anti-ECD3 binding activity. Thus, the combination therapy can optionally comprise administration of a first binding molecule or other therapeutic agent of the present invention which binds specifically to US28 primarily, or only, against an epitope within SEQ ID NO: 177, and a second binding molecule or other therapeutic agent which binds specifically to US28 primarily, or only, against an epitope within ECD3 of US28, such as to an ECD3 epitope as described herein. The present invention also provides formulations comprising the combination of such binding molecules or other therapeutic agents.

### 1. Chemotherapy

Chemotherapeutic anti-cancer agents may, for example, be selected from alkylating agents including nitrogen mustards such as mechlorethamine (HN₂), cyclophosphamide, ifosfamide, melphalan (L-sarcolysin) and chlorambucil; ethylenimines and methylmelamines such as hexamethylmelamine, thiotepa; alkyl sulphonates such as busulphan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxanide); antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin); natural products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes; miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; cell cycle inhibitors; proteosome inhibitors such as Bortezomib (Velcade®); signal transductase (e.g. tyrosine kinase) inhibitors such as Imatinib (Glivec®), COX-2 inhibitors, and hormone agonists/antagonists such as flutamide and tamoxifen.

The clinically used anti-cancer agents are typically grouped by mechanism of action: Alkylating agents, Topoisomerase I inhibitors, Topoisomerase II inhibitors, RNA/DNA antimetabolites, DNA antimetabolites and Antimitotic agents. The US NIH/National Cancer Institute website lists 122 compounds (http://dtp.nci.nih.gov/docs/cancer/_searches/standard_mechanis m.html), all of which may be used in conjunction with US28-target approaches as described by the various aspects of the present invention. They include Alkylating agents including Asaley, AZQ, BCNU, Busulfan, carboxyphthalatoplatinum, CBDCA, CCNU, CHIP, chlorambucil, chlorozotocin, cis-platinum, clomesone, cyanomorpholino-doxorubicin, cyclodisone, dianhydrogalactitol, fluorodopan, hepsulfam, hycanthone, melphalan, methyl CCNU, mitomycin C, mitozolamide, nitrogen mustard, PCNU, piperazine, piperazinedione, pipobroman, porfiromycin, spirohydantoin mustard, teroxirone, tetraplatin, picoplatin (SP-4-3) (cis-aminedichloro(2-methylpyridine)Pt(II)), thio-tepa, triethylenemelamine, uracil nitrogen mustard, Yoshi-864; anitmitotic agents including allocolchicine, Halichondrin B, colchicine, colchicine derivative, dolastatin 10, maytansine, rhizoxin, taxol, taxol derivative, thiocolchicine, trityl cysteine, vinblastine sulphate, vincristine sulphate; Topoisomerase I Inhibitors including camptothecin, camptothecin, Na salt, aminocamptothecin, camptothecin derivatives, morpholinodoxorubicin; Topoisomerase II Inhibitors including doxorubicin, amonafide, m-AMSA, anthrapyrazole derivative, pyrazoloacridine, bisantrene HCL, daunorubicin, deoxydoxorubicin, mitoxantrone, menogaril, N,N-dibenzyl daunomycin, oxanthrazole, rubidazone, VM-26, VP-16; PNA/DNA antimetabolites including L-alanosine, 5-azacytidine, 5-fluorouracil, acivicin, 3 aminopterin derivatives, an antifol, Baker's soluble antifol, dichlorallyl lawsone, brequinar, ftorafur (pro-drug), 5,6-dihydro-5-azacytidine, methotrexate, methotrexate derivative, N-(phosphonoacetyl)-L-aspartate (PALA), pyrazofurin, trimetrexate; DNA antimetabolites including, 3-HP, 2′-deoxy-5-fluorouridine, 5-HP, alpha-TGDR, aphidicolin glycinate, ara-C, 5-aza-2′-deoxycytidine, beta-TGDR, cyclocytidine, guanazole, hydroxyurea, inosine glycodialdehyde, macbecin II, pyrazoloimidazole, thioguanine and thiopurine.

It is, however, preferred that the at least one further anti-cancer agent is selected from cisplatin; carboplatin; picoplatin; 5-flurouracil; paclitaxel; mitomycin C; doxorubicin; gemcitabine; tomudex; pemetrexed; methotrexate; irinotecan, fluorouracil and leucovorin; oxaliplatin, 5-fluorouracil, epirubin, cabecitabine, doxotaxel, paclitaxel and carboplatin.

Preferred anti-angiogenesis compounds include bevacizumab (Avastin®); itraconazole; carboxyamidotriazole; TNP-470 (an analog of fumagillin); CM101; IFN-α; IL-12; platelet factor-4; suramin; SU5416; thrombospondin; VEGFR antagonists; angiostatic steroids+heparin; Cartilage-Derived Angiogenesis Inhibitory Factor; matrix metalloproteinase inhibitors; angiostatin; endostatin; 2-methoxyestradiol; tecogalan; tetrathiomolybdate; thalidomide; prolactin; α_{V}β₃ inhibitors; linomide; tasquinimod; ranibizumab; sorafenib; (Nexavar®); sunitinib (Sutent®); pazopanib (Votrient®); and everolimus (Afinitor®).

The further therapeutic agent may be a hypoxia-activated cytotoxic agent (e.g. Tirapazamine).

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing.

The combination of chemotherapy with biological therapy is known as biochemotherapy. The present invention contemplates any chemotherapeutic agent that may be employed or known in the art for treating or preventing cancers.

### 2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic agent and a chemotherapeutic or radiotherapeutic agent are delivered to a target or are placed in direct juxtaposition with the target. To achieve cell killing or stasis, both agents are delivered to a cell in a combined amount effective to kill one or more cells at the target or prevent said cells from dividing.

### 3. Immunotherapy

Immunotherapeutics, generally, rely on the use of immune effector cells and molecules to target and destroy cancer cells. The immune effector may be, for example, an antibody specific for some marker on the surface of a tumor cell. For the purposes of the present invention, and without limitation, the marker can be US28 as expressed on the surface of HCMV-infected tumor cells; in which case the antibody can be, or comprise, any of the binding molecules of the present invention. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T-cells and NK cells. The combination of therapeutic modalities, i.e., direct cytotoxic activity and inhibition or reduction of Fortilin would provide therapeutic benefit in the treatment of cancer.

Immunotherapy could also be used as part of a combined therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as mda-7 has been shown to enhance anti-tumor effects (Ju et al., 2000).

Examples of immunotherapies currently under investigation or in use are immune adjuvants (e.g., Mycobacterium bovis, Plasmodium falciparum, dinitrochlorobenzene and aromatic compounds) (U.S. Pat. Nos. 5,801,005; 5,739,169; Hui and Hashimoto, 1998, Infect. Immun., 66(11):5329-36; Christodoulides et al., 1998, Microbiology, 144(Pt II):3027-37), cytokine therapy (e.g., interferons, and; IL-1, GM-CSF and TNF) (Bukowski et al., 1998, Clin. Cancer Res., 4(10):2337-47; Davidson et al., 1998,J. Immunother., 21(5):389-98; Hellstrand et al., 1998, Oncol, 37(4):347-353), gene therapy (e.g., TNF, IL-1, IL-2, p53) (Qin et al, 1998, Proc. Natl. Acad. Sci. USA, 95(24): 1441 1-14416; Austin-Ward and Villaseca, 1998,Rev. Med. Chil, 126(7):838-45; U.S. Pat. Nos. 5,830,880 and 5,846,945) and monoclonal antibodies (e.g., anti-ganglioside GM2, anti-HER−2, anti-p185) (Pietras et al., 1998, Oncogene, 17(17):2235-49; Hanibuchi et al., 1998, Int J. Cancer, 78(4):480-5; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999, Cancer Biother. Radiopharm., 14:5-10). Combination therapy of cancer with herceptin and chemotherapy has been shown to be more effective than the individual therapies. Thus, it is contemplated that one or more anti-cancer therapies may be employed with the therapies of the present invention directed to ECD3 of HCMV-encoded US28, as described herein.

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988, N. Engl J. Med, 319:1676; Rosenberg et al, 1989, Ann. Surg., 210:474). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders was few compared to those who did not respond.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie & Morton, 1986, Proc. Nat Acad. Sci. USA 83:8694-8698). Regression was observed in six out of ten patients, following, daily or weekly, intralesional injections. In another study, moderate success was achieved from intralesional injections of two human monoclonal antibodies (Irie et al., “Melanoma gangliosides and human monoclonal antibody” In: Human Tumor Antigens and Specific Tumor Therapy, Metzgar & Mitchell (eds.), Alan R. Liss, Inc., New York, pp. 115-126, 1989). Possible therapeutic antibodies include anti-TNF, anti-CD25, anti-CD3, anti-CD20, CTLA-4-IG, and anti-CD28.

It may be favorable to administer more than one monoclonal antibody directed against two different antigens or even antibodies with multiple antigen specificity. Treatment protocols also may include administration of lymphokines or other immune enhancers as described by Bajorin et al., 1988, Proc. Anu. Meet. Am. Soc. Clin. Oncol, 7:A967. The development of human monoclonal antibodies is described in further detail elsewhere in the specification.

### 4. Gene Therapy

In yet another embodiment, the secondary treatment is a gene therapy in which a therapeutic polynucleotide is administered before, after, or at the same time as the therapy of the eleventh aspect of the present invention. Various genes that may be targeted for gene therapy of some form in combination with the present invention are well known to one of ordinary skill in the art and may comprise any gene involved in cancers.

Inducers of Cellular Proliferation. The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abi and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity. The proteins Jun, Fos and Myc are proteins that directly exert their effects on nuclear functions as transcription factors.

Inhibitors of Cellular Proliferation. The tumor suppressor oncogenes function to inhibit excessive cellular proliferation. The inactivation of these genes destroys their inhibitory activity, resulting in unregulated proliferation. The most common tumor suppressors are Rb, p53, p21 and p16. Other genes that may be employed according to the present invention include APC, DCC, NF-1, NF-2, WT-1, MEN-I, MEN-II, zac1, p73, VHL, C-CAM, MMAC1/PTEN, DBCCR-1, FCC, rsk-3, p27, p27/p16 fusions, and p21/p27 fusions.

Regulators of Programmed Cell Death. Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985, Cell, 41(3):899-906; Cleary and Sklar, 1985, Proc. Natl Acad. Sci. USA, 82(21):7439-43; Cleary et al., 1986, J Exp. Ned, 164(1):315-20; Tsujimoto et al., 1985, Science, 228(4706):1440-3; Tsujimoto and Croce, 1986, Proc. Natl Acad. Sci. USA, 83(14):5214-8). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins that share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Blk, Bim, Bid, Bad, Harakiri).

### 5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months.

I. ECD1- and/or ECD3-Derived Peptide Sequences and Uses Thereof

As discussed, above, when considering the design of binding molecules against US28, the inventor noted, whereas the N-terminal part (ECD1) of the US28 protein contains high levels of mutations amongst the different HCMV strains, a specific region of ECD1 was identified by the inventor that could be an appropriate target for a highly specific, and strain agnostic, antibody.

This approach was based on the use of a peptide sequence derived from ECD1 having the sequence TDVLNQSKPVTL (SEQ ID NO: 177).

This peptide sequences, which is referred to as Peptide-N in the Examples of the present application, was used to immunise mice, and to screen for and identify antibodies that had highly specific, and likely strain agnostic, binding properties for genetic variants of the US28 ECD1, as well as showing highly-specific binding to US28 overexpressing US28-CHO-A1 cells, and HCMV infected human lung tissue.

The inventor previously found that the conserved ECD2 and the conserved parts of ECD4 protein were not appropriate immunogens in mice, and so the applicant previously developed an approach to the identification of highly specific, strain agnostic, binding molecules against the ECD3 region of HCMV US28.

This approach was based on the use of peptide sequences derived from the two variant forms of ECD3, the 4N-variant having the sequence TKKNNQCMTDYDYLEVS, as defined by SEQ ID NO: 6; and the 4D-variant having the sequence TKKDNQCMTDYDYLEVS, as defined by SEQ ID NO: 7.

These peptide sequences, which are referred to as Peptides 1 and 2, respectively, in the Examples of the present application, were used to immunise mice, and to screen for and identify antibodies that had highly specific, yet strain agnostic, binding properties for both genetic variants of the US28 ECD3 peptides, as well as showing highly-specific binding to US28 overexpressing US28-CHO-A1 cells, HCMV Ad169 infected MRC-5 cells, primary PBMCs from HCMV seropositive individuals, HCMV infected human lung tissue and several types of aggressive human tumors, such as oesophagus, gastric, rectum, liver, lung, pancreas, cervical cancers, malignant pheochromocytoma and locally advanced colon cancer, breast cancer and its metastasis and glioblastoma grade 4.

Said peptide sequences, and functional derivatives and variants thereof, as well as their uses in accordance with the present invention, are further provided.

As previously mentioned, a thirteenth aspect of the present invention provides a peptide or polypeptide comprising, consisting essentially of, or consisting of, the sequence of TDVLNQSKPVTL (SEQ ID NO: 177).

Said peptide or polypeptide is not the US28 protein. For example, outside of the sequence defined by SEQ ID NO: 177 or the immunogenic fragment or variant thereof, the remaining sequence of the peptide or polypeptide of the thirteenth aspect of the present invention may have less than 50%, 40%, 30%, 20%, 10%, or 5% sequence identity to the sequence of US28, when considered over the entirety of its length. Preferably the only US28-derived sequence in said peptide or polypeptide is the sequence of SEQ ID NO: 177 or the sequence of the immunogenic fragment or variant of SEQ ID NO: 177.

Furthermore, a fourteenth aspect of the present invention provides a peptide or polypeptide comprising, consisting essentially of, or consisting of, the sequence of the 4N-variant of ECD3 of US28, TKKNNQCMTDYDYLEVS (SEQ ID NO: 6), or comprising the sequence of an immunogenic fragment or variant of SEQ ID NO: 6.

Said peptide or polypeptide is not the US28 protein. For example, outside of the sequence defined by SEQ ID NO: 6 or the immunogenic fragment or variant thereof, the remaining sequence of the peptide or polypeptide of the thirteenth aspect of the present invention may have less than 50%, 40%, 30%, 20%, 10%, or 5% sequence identity to the sequence of US28, when considered over the entirety of its length. Preferably the only US28-derived sequence in said peptide or polypeptide is the sequence of SEQ ID NO: 6 or the sequence of the immunogenic fragment or variant of SEQ ID NO: 6.

The fourteenth aspect of the present invention also provides a peptide or polypeptide comprising, consisting essentially of, or consisting of, the sequence TKKDNQCMTDYDYLEVS (SEQ ID NO:7), or comprising the sequence of an immunogenic fragment or variant of SEQ ID NO: 7.

Said peptide or polypeptide is not the US28 protein. For example, outside of the sequence defined by SEQ ID NO: 7 or the immunogenic fragment or variant thereof, the remaining sequence of the peptide or polypeptide of the fourteenth aspect of the present invention may have less than 50%, 40%, 30%, 20%, 10%, or 5% sequence identity to the sequence of US28, when considered over the entirety of its length. Preferably the only US28-derived sequence in said peptide or polypeptide is the sequence of SEQ ID NO: 7 or the sequence of the immunogenic fragment or variant of SEQ ID NO: 7.

An immunogenic fragment or variant of the reference sequence SEQ ID NO: 177, in accordance with the thirteenth aspect of the present invention may comprise less than the full sequence of the reference sequence, and preferably comprises at least 3, 4, 5, 6, 7, 8, 9, 10 or 11 consecutive amino acids of the reference sequence. Said immunogenic fragment of SEQ ID NO: 177 may, for example, be selected from any one or more of:
An 11-mer selected from: DVLNQSKPVTL; or TDVLNQSKPVT;
A 10-mer selected from: VLNQSKPVTL; DVLNQSKPVT or TDVLNQSKPV;
A 9-mer selected from: LNQSKPVTL; VLNQSKPVT; DVLNQSKPV; or TDVLNQSKP;
A 8-mer selected from: NQSKPVTL; LNQSKPVT; VLNQSKPV; DVLNQSKP; or TDVLNQSK;
A 7-mer selected from: QSKPVTL; NQSKPVT; LNQSKPV; VLNQSKP; DVLNQSK; or TDVLNQS;
A 6-mer selected from: SKPVTL; QSKPVT; NQSKPV; LNQSKP; VLNQSK; DVLNQS; or TDVLNQ;
A 5-mer selected from: KPVTL; SKPVT; QSKPV; NQSKP; LNQSK; VLNQS; DVLNQ; or TDVLN;
A 4-mer selected from: PVTL; KPVT; SKPV; QSKP; NQSK; LNQS; VLNQ; DVLN; or TDVL; or
A 3-mer selected from: VTL; PVT; KPV; SKP; QSK; NQS; LNQ; VLN; DVL; TDV.

An immunogenic fragment or variant of the reference sequence SEQ ID NO: 6 or 7, in accordance with the fourteenth aspect of the present invention, respectively, comprises less than the full sequence of the reference sequence, and preferably comprises at least 3, 4, 5, 6, 7, 8, 9, 10, 11, 12, 13, 14, 15, or 16 consecutive amino acids of the reference sequence.

Said immunogenic fragment of SEQ ID NO: 6 may, for example, be selected from any one or more of:
A 16-mer selected from: KKNNQCMTDYDYLEVS or TKKNNQCMTDYDYLEV;
A 15-mer selected from: KNNQCMTDYDYLEVS; KKNNQCMTDYDYLEV; or TKKNNQCMTDYDYLE;
A 14-mer selected from: NNQCMTDYDYLEVS; KNNQCMTDYDYLEV; KKNNQCMTDYDYLE; or TKKNNQCMTDYDYL;
A 13-mer selected from: NQCMTDYDYLEVS; NNQCMTDYDYLEV; KNNQCMTDYDYLE; KKNNQCMTDYDYL; or TKKNNQCMTDYDY;
A 12-mer selected from: QCMTDYDYLEVS; NQCMTDYDYLEV; NNQCMTDYDYLE; KNNQCMTDYDYL; KKNNQCMTDYDY; TKKNNQCMTDYD;
An 11-mer selected from: CMTDYDYLEVS; QCMTDYDYLEV; NQCMTDYDYLE; NNQCMTDYDYL; KNNQCMTDYDY; KKNNQCMTDYD; or TKKNNQCMTDY;
A 10-mer selected from: MTDYDYLEVS; CMTDYDYLEV; QCMTDYDYLE; NQCMTDYDYL; NNQCMTDYDY; KNNQCMTDYD; KKNNQCMTDY; or TKKNNQCMTD;
A 9-mer selected from: TDYDYLEVS; MTDYDYLEV; CMTDYDYLE; QCMTDYDYL; NQCMTDYDY; NNQCMTDYD; KNNQCMTDY; KKNNQCMTD; or TKKNNQCMT;
A 8-mer selected from: DYDYLEVS; TDYDYLEV; MTDYDYLE; CMTDYDYL; QCMTDYDY; NQCMTDYD; NNQCMTDY; KNNQCMTD; KKNNQCMT; or TKKNNQCM;
A 7-mer selected from: YDYLEVS; DYDYLEV; TDYDYLE; MTDYDYL; CMTDYDY; QCMTDYD; NQCMTDY; NNQCMTD; KNNQCMT; KKNNQCM; or TKKNNQC;
A 6-mer selected from: DYLEVS; YDYLEV; DYDYLE; TDYDYL; MTDYDY; CMTDYD; QCMTDY; NQCMTD; NNQCMT; KNNQCM; KKNNQC; or TKKNNQ;
A 5-mer selected from: YLEVS; DYLEV; YDYLE; DYDYL; TDYDY; MTDYD; CMTDY; QCMTD; NQCMT; NNQCM; KNNQC; KKNNQ; orTKKNN;
A 4-mer selected from: LEVS; YLEV; DYLE; YDYL; DYDY; TDYD; MTDY; CMTD; QCMT; NQCM; NNQC; KNNQ; KKNN; orTKKN; or
A 3-mer selected from: EVS; LEV; YLE; DYL; YDY; DYD; TDY; MTD; CMT; QCM; NQC; NNQ; KNN; KKN; TKK.

Said immunogenic fragment of SEQ ID NO: 7 may, for example, be selected from any one or more of:
A 16-mer selected from: KKDNQCMTDYDYLEVS or TKKDNQCMTDYDYLEV;
A 15-mer selected from: KDNQCMTDYDYLEVS; KKDNQCMTDYDYLEV; or TKKDNQCMTDYDYLE;
A 14-mer selected from: DNQCMTDYDYLEVS; KDNQCMTDYDYLEV; KKDNQCMTDYDYLE; or TKKDNQCMTDYDYL;
A 13-mer selected from: NQCMTDYDYLEVS; DNQCMTDYDYLEV; KDNQCMTDYDYLE; KKDNQCMTDYDYL; or TKKDNQCMTDYDY;
A 12-mer selected from: QCMTDYDYLEVS; NQCMTDYDYLEV; DNQCMTDYDYLE; KDNQCMTDYDYL; KKDNQCMTDYDY; TKKDNQCMTDYD;
An 11-mer selected from: CMTDYDYLEVS; QCMTDYDYLEV; NQCMTDYDYLE; DNQCMTDYDYL; KDNQCMTDYDY; KKDNQCMTDYD; or TKKDNQCMTDY;
A 10-mer selected from: MTDYDYLEVS; CMTDYDYLEV; QCMTDYDYLE; NQCMTDYDYL; DNQCMTDYDY; KDNQCMTDYD; KKDNQCMTDY; or TKKDNQCMTD;
A 9-mer selected from: TDYDYLEVS; MTDYDYLEV; CMTDYDYLE; QCMTDYDYL; NQCMTDYDY; DNQCMTDYD; KDNQCMTDY; KKDNQCMTD; or TKKDNQCMT;
A 8-mer selected from: DYDYLE-VS; TDYDYLEV; MTDYDYLE; CMTDYDYL; QCMTDYDY; NQCMTDYD; DNQCMTDY; KDNQCMTD; KKDNQCMT; or TKKDNQCM;
A 7-mer selected from: YDYLEVS; DYDYLEV; TDYDYLE; MTDYDYL; CMTDYDY; QCMTDYD; NQCMTDY; DNQCMTD; KDNQCMT; KKDNQCM; or TKKDNQC;
A 6-mer selected from: DYLEVS; YDYLEV; DYDYLE; TDYDYL; MTDYDY; CMTDYD; QCMTDY; NQCMTD; DNQCMT; KDNQCM; KKDNQC; or TKKDNQ;
A 5-mer selected from: YLEVS; DYLEV; YDYLE; DYDYL; TDYDY; MTDYD; CMTDY; QCMTD; NQCMT; DNQCM; KDNQC; KKDNQ; or TKKDN;
A 4-mer selected from: LEVS; YLEV; DYLE; YDYL; DYDY; TDYD; MTDY; CMTD; QCMT; NQCM; DNQC; KDNQ; KKDN; or TKKD; or
A 3-mer selected from: EVS; LEV; YLE; DYL; YDY; DYD; TDY; MTD; CMT; QCM; NQC; DNQ; KDN; KKD; TKK.

Preferably, the or each of the immunogenic fragments selected exclude those sequences present in any other proteins, in particular in the extracellular region of any proteins, such as other GPCRs; this can aid in obtaining the required specificity for ECD1 and/or ECD3 of US28, and minimising or preferably avoiding, any off-target effects. Accordingly, longer immunogenic fragments may be preferred, for example at least 5-mers, at least 6-mers, at least 7-mers, at least 8-mers, at least 9-mers, at least 10-mers, at least 11-mers, at least 12-mers, at least 13-mers, at least 14-mers, at least 15-mers, or at least 16-mers.

An immunogenic fragment or variant of the reference sequence SEQ ID NO: 177, 6 or 7, in accordance with the thirteenth or fourteenth aspect of the present invention, respectively, is typically capable of provoking an immune response (e.g. an immune response in the body of a human or other animal), such as by provoking a humoral and/or cell-mediated immune response. Said immune response is one that has specificity to the reference sequence (i.e. SEQ ID NO: 177, 6 and/or 7) as present within a peptide or polypeptide according to the thirteenth or fourteenth aspect of the present invention, respectively.

In one embodiment, an immunogenic fragment or variant of the peptide or polypeptide of the fourteenth aspect of the present invention, comprises, consists essentially of, or consists of, a sequence that is common to, and present within, both of SEQ ID NO: 6 and SEQ ID NO: 7. Said common sequence can comprise, consist essentially of, or consist of, a continuous amino acid sequence or a non-continuous amino acid sequence.

In one embodiment, the immune response that can be provoked by an immunogenic fragment or variant of the peptide or polypeptide of the fourteenth aspect of the present invention, can be an immune response that has specificity to a sequence that is common to, and present within, both of SEQ ID NO: 6 and SEQ ID NO: 7. For example, the common sequence can comprise, consist essentially of, or consist of, a continuous amino acid sequence or a non-continuous amino acid sequence that is common to, and present within, both of SEQ ID NO: 6 and SEQ ID NO: 7.

Also provided by the thirteenth and fourteenth aspects of the present invention are peptides or polypeptides comprising, consisting, or consisting essentially of, an immunogenic fragment or variant of a reference sequence selected from TDVLNQSKPVTL (SEQ ID NO: 177), TKKNNQCMTDYDYLEVS (SEQ ID NO: 6), or TKKDNQCMTDYDYLEVS (SEQ ID NO: 7), respectively, wherein the immunogenic fragment or variant comprises, consists essentially of, or consists of, the sequence of the epitope within ECD1 and/or ECD3 of US28 that is bound by antibody 4H3C3, 7B1F3, 2F5B11, 1D3, 1C10, 1A10, 1G9 and/or 1E8 (by which is included also an scFv comprising a V_{H} polypeptide sequence having the V_{H} sequence of 4H3C3, 7B1F3, 2F5B11, 4A11A11, 1D3, 1C10, 1A10, 1G9 and/or 1E8 as defined by SEQ ID No: 187, 211, 233, 12, 104, 122, 68 and 88, respectively, and a V_{L} polypeptide sequence having the V_{L} sequence of 4H3C3, 7B1F3, 2F5B11, 1D3, 1C10, 1A10, 1G9 and/or 1E8 as defined by SEQ ID No: 189, 213, 235, 18, 108, 126, 72 and 92, respectively). Preferably said peptides or polypeptides are bound specifically by 4H3C3, 7B1F3, 2F5B11, 1D3, 1C10, 1A10, 1G9 and/or 1E8.

In one embodiment, the (or each of the) peptide(s) or polypeptide(s) of the thirteenth and/or fourteenth aspect of the present invention (and/or immunogenic fragment or variant thereof) is or are provided in a purified and/or isolated form.

The peptides or polypeptides (or immunogenic fragments or variants thereof) as defined by the thirteenth and/or fourteenth aspects of the present invention are useful in a wide variety of roles and utilities. Without limitation, they may be used separately, or in combination, to provoke an immune reaction with specificity against ECD1 and/or ECD3 of the US28 protein of HCMV (e.g. as a vaccine in a subject and/or to generate further antibodies with binding specificity against ECD1 and/or ECD3 of US28); and also to capture, identify and/or characterise binding molecules having specify to ECD1 and/or ECD3 of US28 (including, for example, by providing a means to capture, identify and/or characterise such binding molecules that have strain agnostic binding specify to ECD1 and/or ECD3 of US28, such that said strain agnostic binding molecules can be confirmed to bind essentially equally to each of the peptides or polypeptides (or immunogenic fragments or variants thereof) as defined by the thirteenth and/or fourteenth aspects of the present invention irrespective of the presence of known ECD1 mutations (e.g. ECD1 mutations described in table 3), and/or the 4N-variant sequence or the 4D-variant sequence). Accordingly, it may be useful in some embodiments to use said peptides or polypeptides (or immunogenic fragments or variants thereof) as defined by the thirteenth and/or fourteenth aspects of the present invention in combination, either separately (e.g. one of each type in a separate assay, said separate assays to be used in combination), or in admixture with each other.

As previously mentioned, a fifteenth aspect of the present invention provides a combination of at least two (e.g. three) distinct peptides and/or polypeptides, comprising a first peptide or polypeptide and a second peptide or polypeptide (and optionally a third polypeptide), wherein:
(a) the first peptide or polypeptide comprises a comprises, consists essentially of, or consists of, the sequence TDVLNQSKPVTL (SEQ ID NO: 177), or an immunogenic fragment thereof, such as an immunogenic fragment as defined by the thirteenth aspect of the present invention; and
(b) the second peptide or polypeptide comprises a comprises, consists essentially of, or consists of, the sequence TKKNNQCMTDYDYLEVS (SEQ ID NO: 6), or an immunogenic fragment or variant thereof, such as a peptide or polypeptide (or an immunogenic fragment or variant thereof) as defined by the fourteenth aspect of the present invention, with the proviso that said immunogenic fragment comprises at least the 4N amino acid of SEQ ID NO: 6; or
(c) the second peptide or polypeptide comprises a comprises, consists essentially of, or consists of, the sequence TKKDNQCMTDYDYLEVS (SEQ ID NO: 7), or an immunogenic fragment thereof, such as an immunogenic fragment as defined by the fourteenth aspect of the present invention, with the proviso that said immunogenic fragment comprises at least the 4D amino acid of SEQ ID NO: 7; and
optionally wherein the third peptide or polypeptide corresponds to option (b) or (c), and wherein the third peptide or polypeptide is distinct from the second peptide or polypeptide.

The peptides or polypeptides (or immunogenic fragments or variants thereof) as defined by the thirteenth and/or fourteenth aspects of the present invention, and the combination of at least two (e.g. three) distinct peptides and/or polypeptides as defined by the fifteenth aspect of the present invention, can be presented in the most appropriate manner for their utility. For example, it may be suitable to create fusion proteins or conjugates (which, without limitation, can be suitable as carriers for presenting said peptides or polypeptides (or immunogenic fragments or variants thereof) to the immune system), and/or to create immobilised versions of said peptides or polypeptides (or immunogenic fragments or variants thereof), or fusion proteins or conjugates thereof, for example for the use of said immobilised molecules in assays for the capture, identification and/or characterisation of binding molecules having specify to ECD1 and/or ECD3 of US28.

Accordingly, a sixteenth aspect of the present invention provides a fusion protein comprising, consisting essentially of, or consisting of, a first amino acid sequence fused, either directly or via one or more linker amino acid sequences, to a second amino acid sequence, wherein the first amino acid sequence is the sequence of a peptide or polypeptide (or immunogenic fragment or variant thereof) as defined by the thirteenth or fourteenth aspect of the present invention; and the second amino acid sequence is a fusion partner. Preferably, said fusion protein is not the US28 protein. Preferably the only US28-derived sequence in said fusion protein is the sequence of SEQ ID NO: 177 or the sequence of the immunogenic fragment of SEQ ID NO: 177 and/or said fusion protein does not contain any sequence of at 3, 4, 5, 6, 7, 8, 9, 10 or more than 10 contiguous amino acids that is identical to a sequence of contiguous amino acid sequence of equivalent length found in the US28 protein at a location other than the region comprising the sequence of SEQ ID NO: 177

Any fusion partner of interest may be selected. The skilled person is well aware of fusion partner sequences known in the art, and any may be selected. A fusion partner may, for example, be a carrier protein. Additionally, or alternatively, the fusion partner may provide an additional or alternative binding property and/or antigenic property to the first amino acid sequence to which it is fused; it may provide an effector portion that creates an effect at the site of binding of the first amino acid sequence; it may provide for a modulation (such as an increase or decrease) in the circulatory half-life of the binding molecule; it may provide a sequence that facilitates the detection of the binding molecule.

In one preferred option, the fusion partner is a carrier protein, such as a carrier protein that is selected to provide a fusion protein that is suitable for immunisation and generation of antibodies against the first amino acid sequence. For example, the carrier protein may be selected from the group consisting of keyhole limpet hemocyanin (KLH), HSA (human serum albumin), BSA (bovine serum albumin), OVA (ovalbumin), tetanus toxoid (TT), diphtheria toxoid (DT), a genetically modified cross-reacting material (CRM) of diphtheria toxin, meningococcal outer membrane protein complex (OMPC) and H. influenzae protein D (HiD).

To the best of the applicant's knowledge, to date, the five carrier proteins that have been used in licensed conjugate vaccines are: TT, DT, a genetically modified CRM of diphtheria toxin, meningococcal OMPC and HiD. Clinical trials have demonstrated the efficacy of these conjugate vaccines in preventing infectious diseases. All five of the carrier proteins are effective at increasing the immunogenicity of the vaccine but elicit different amounts of antibodies with different affinities. Additional carrier proteins that have been evaluated, albeit less extensively in clinical trials, include rEPA (Pseudomonas aeruginosa exotoxin A), KLH (keyhole limpet hemocyanin), and flagellin.

A seventeenth aspect of the present invention provides a combination of at least two (e.g. three) distinct fusion proteins, comprising a first fusion protein, and a second fusion protein (and optionally a third fusion protein), wherein:
(a) the first fusion protein according to the sixteenth aspect of the present invention comprises, as the first amino acid sequence of the first fusion protein, a sequence that comprises a comprises, consists essentially of, or consists of, the sequence TDVLNQSKPVTL (SEQ ID NO: 177), or an immunogenic fragment or variant thereof; and
(b) the second fusion protein according to the sixteenth aspect of the present invention comprises, as the first amino acid sequence of the first fusion protein, a sequence that comprises a comprises, consists essentially of, or consists of, the sequence TKKNNQCMTDYDYLEVS (SEQ ID NO: 6), or an immunogenic fragment or variant thereof, with the proviso that said immunogenic fragment or variant includes the 4N amino acid of SEQ ID NO: 6; or
has the second fusion protein according to the sixteenth aspect of the present invention comprises, as the first amino acid sequence of the second fusion protein, a sequence that comprises, consists essentially of, or consists of, the sequence TKKDNQCMTDYDYLEVS (SEQ ID NO: 7), or an immunogenic fragment or variant thereof, with the proviso that said immunogenic fragment or variant includes the 4D amino acid of SEQ ID NO: 7; and
optionally the third fusion protein according to option (b) or (c), wherein the third fusion protein is distinct from the second fusion protein.

Said combination can, for example, be particularly useful as the immunogenic component of a vaccine composition that can be used for provoking an immune response in a subject that has reactivity to ECD1 in multiple strains of the HCMV virus with varying ECD1 sequences; and/or against both 4N-variant and 4D-variant strains of the HCMV virus. Said response can, for example, include generating a polyclonal antibody response that comprises a population of antibodies including antibodies with binding specificity to the 4N-variant of ECD3 of US28, antibodies with binding specificity to the 4D-variant of ECD3 of US28 and preferably antibodies with strain-agnostic binding specificity to both of the 4N- and 4D-variants of ECD3 of US28. The present invention also provides an isolated polyclonal antibody product that can be obtained from said polyclonal antibody response, wherein said polyclonal antibody product can be selected to comprise, consist essentially of, or consist of antibodies having binding specificity to ECD1; and optionally to either, or preferably both, of the 4N- and 4D-variants of ECD3 of US28.

As discussed above, as an alternative (or additional modification) to the creation of fusion proteins, the present invention also provides for the production of conjugates. Accordingly, an eighteenth aspect of the present invention provides a conjugate, comprising a moiety conjugated to a peptide or polypeptide as defined by either or both of the thirteenth and fourteenth aspects of the present invention, or to a fusion protein as defined by the sixteenth aspect of the present invention.

The moiety of said conjugate may be conjugated directly to the peptide or polypeptide (or immunogenic fragment or variant thereof) as defined by either or both of the thirteenth and fourteenth aspects of the present invention, or to the fusion protein as defined by the sixteenth aspect of the present invention. Alternatively, the moiety of said conjugate may be conjugated indirectly, such as via a linker, to the peptide or polypeptide as defined by either or both of the thirteenth and fourteenth aspects of the present invention, or to the fusion protein as defined by the sixteenth aspect of the present invention.

Optionally, the moiety may be a carrier, for example a carrier protein, such as a carrier selected from KLH (keyhole limpet hemocyanin), HSA (human serum albumin), BSA (bovine serum albumin), OVA (ovalbumin), tetanus toxoid (TT), diphtheria toxoid (DT), a genetically modified cross-reacting material (CRM) of diphtheria toxin, meningococcal outer membrane protein complex (OMPC) and H. influenzae protein D (HiD).

A nineteenth aspect of the present invention provides a combination of at least two (e.g. three) distinct conjugates, wherein the combination comprises:
(a) a first conjugate according to the eighteenth aspect of the present invention, wherein the first conjugate comprises, consists essentially of, or consists of, a moiety conjugated to a peptide or polypeptide, wherein the peptide or polypeptide comprises, consists essentially of, or consists of, the sequence TDVLNQSKPVTL (SEQ ID NO: 177), or an immunogenic fragment or variant thereof; and
(b) a second conjugate according to the eighteenth aspect of the present invention, wherein the first conjugate comprises, consists essentially of, or consists of, a moiety conjugated to a peptide or polypeptide, wherein the peptide or polypeptide comprises, consists essentially of, or consists of, the sequence TKKNNQCMTDYDYLEVS (SEQ ID NO: 6), or an immunogenic fragment or variant thereof, with the proviso that said immunogenic fragment or variant includes the 4N amino acid of SEQ ID NO: 6; or
has a second conjugate according to the eighteenth aspect of the present invention, wherein the second conjugate comprises, consists essentially of, or consists of, a moiety conjugated to a peptide or polypeptide, wherein the peptide or polypeptide comprises, consists essentially of, or consists of, the sequence TKKDNQCMTDYDYLEVS (SEQ ID NO: 7), or an immunogenic fragment or variant thereof, with the proviso that said immunogenic fragment or variant includes the 4D amino acid of SEQ ID NO: 7; and
optionally a third conjugate according to option (b) or (c), wherein the third conjugate is distinct from the second conjugate.

Said combination can, for example, be particularly useful as the immunogenic component of a vaccine composition that can be used for provoking an immune response in a subject that has reactivity against multiple strains of the HCMV virus with varying ECD1 sequences; and optionally against both 4N-variant and 4D-variant strains of the HCMV virus. Said response can, for example, include generating a polyclonal antibody response that comprises a population of antibodies including antibodies with binding specificity to ECD1; and optionally to the 4N-variant of ECD3 of US28, antibodies with binding specificity to the 4D-variant of ECD3 of US28 and preferably antibodies with strain-agnostic binding specificity to both of the 4N- and 4D-variants of ECD3 of US28. The present invention also provides an isolated polyclonal antibody product that can be obtained from said polyclonal antibody response, wherein said polyclonal antibody product can be selected to comprise, consist essentially of, or consist of antibodies having binding specificity to ECD1; and optionally to either, or preferably both, of the 4N- and 4D-variants of ECD3 of US28.

A twentieth aspect of the present invention provides a method of producing a conjugate according to the nineteenth aspect of the present invention, the method comprising the steps of: (a) providing a peptide or polypeptide (or an immunogenic fragment or variant thereof) as defined by either or both of the thirteenth and/or fourteenth aspects of the present invention, a combination thereof as defined by the fifteenth aspect of the present invention, or a fusion protein as defined by the sixteenth aspect of the present invention; and (b) conjugating a moiety thereto.

Any means of conjugating a moiety to the peptide or polypeptide (or an immunogenic fragment or variant thereof) may be used; the skilled person is well aware of conventional means of generating conjugates between peptides/polypeptides of choice and other moieties of choice, and are free to select the most appropriate method.

For example, and without limitation, some conventional ways of cross-linking polypeptides include those generally described in O'Sullivan et al, Anal. Biochem., 1979, 100, 100-108. For example, a peptide or polypeptide may be engineered to be enriched with one or more thiol groups and a moiety selected that is suitable for reaction with a bifunctional agent capable of reacting with those thiol groups, for example the N-hydroxysuccinimide ester of iodoacetic acid (NHIA) or N-succinimidyl-3-(2-pyridyldithio)propionate (SPDP), a heterobifunctional cross-linking agent which incorporates a disulphide bridge between the conjugated species. Amide and thioether bonds, for example achieved with m-maleimidobenzoyl-N-hydroxysuccinimide ester, are generally more stable in vivo than disulphide bonds.

Further useful cross-linking agents include, without limitation, S-acetylthioglycolic acid N-hydroxysuccinimide ester (SATA) which is a thiolating reagent for primary amines which allows deprotection of the sulphydryl group under mild conditions (Julian et al, Anal. Biochem., 1983, 132: 68), dimethylsuberimidate dihydrochloride and N,N′-o-phenylenedimaleimide.

A twenty-first aspect of the present invention provides a method of producing a combination of at least two (e.g. three) distinct conjugates as defined by the nineteenth aspect of the present invention. Either the two (optionally three) distinct conjugates can be produced separately, and then combined, or alternatively, two (optionally three) distinct peptides or polypeptides (or an immunogenic fragment or variant of either) can be combined, and then conjugates of each generated in the same reaction in order to produce the combination of at least two (e.g. three) distinct conjugates.

The methods of the twentieth or twenty-first aspect of the present invention optionally comprise the step of isolating the thus-produced conjugate or combination of conjugates; and a twenty-second aspect of the present invention provides an isolated conjugate, or combination of conjugates, that is obtained, or obtainable, by the method of any of twentieth or twenty-first aspect of the present invention, optionally, wherein the isolated conjugate is further formulated for administration to a subject. For example, it may be formulated for a downstream use, for example, for administration to a recipient as a pharmaceutical or veterinary composition.

A twenty-third aspect of the present invention provides nucleic acid molecule, or combination of multiple distinct nucleic acid molecules, wherein the nucleic acid molecule comprises, or the combination of multiple distinct nucleic acid molecules collectively comprises, one or more nucleic acid sequences that, individually or in combination, encode one or more peptides and/or polypeptides according to either or both of the thirteenth and/or fourteenth aspects of the present invention, a combination of at least two (e.g. three) distinct peptides and/or polypeptides according to the fifteenth aspect of the present invention, a fusion protein according to the sixteenth aspect of the present invention, and/or a combination of at least two (e.g. three) distinct fusion proteins according to the seventeenth aspect of the present invention.

The, or each, nucleic acid molecule according to the twenty-third aspect of the present invention may, for example, be each independently selected from a DNA or RNA molecule. The, or each, nucleic acid molecule according to the twenty-third aspect of the present invention may, for example, be each independently selected from a single-stranded or a double-stranded nucleic acid molecule. Each of the options discussed above in Section B of this application, in the context of nucleic acids that encode binding molecules according to the first aspect of the present invention are disclosed herein and can be applied, mutatis mutandis to the, or each, nucleic acid molecule according to the twenty-third aspect of the present invention.

A twenty-fourth aspect of the present invention provides a vector comprising a nucleic acid molecule, or combination of multiple distinct nucleic acid molecules, according to the twenty-third aspect of the present invention. Any vector may be used, although without limitation, said vector may optionally be selected from the group consisting of a retroviral vector, a plasmid, a lentivirus vector, and an adenoviral vector. Each of the options discussed above in Section C of this application, in the context of vectors that incorporate the one or more nucleic acids that encode binding molecules according to the first aspect of the present invention are disclosed herein and can be applied, mutatis mutandis to the, or each, vectors according to the twenty-fourth aspect of the present invention.

A twenty-fifth aspect of the present invention provides a cell comprising the nucleic acid molecule, or combination of multiple distinct nucleic acid molecules, according to the twenty-third aspect of the present invention, or the vector according to the twenty-fourth aspect of the present invention. Optionally, the cell expresses one or more peptide or polypeptide selected from either or both of the thirteenth and/or fourteenth aspects of the present invention, a combination of at least two (e.g. three) distinct peptides and/or polypeptides according to the fifteenth aspect of the present invention, a fusion protein according to the sixteenth aspect of the present invention, and/or a combination of at least two (e.g. three) distinct fusion proteins according to the seventeenth aspect of the present invention. Each of the options discussed above in Section D of this application, in the context of cells that incorporate the one or more nucleic acids that encode binding molecules according to the first aspect of the present invention (or vectors incorporating said or more nucleic acids) are disclosed herein and can be applied, mutatis mutandis to the, or each, cells according to the twenty-fifth aspect of the present invention.

The present invention further provides an isolated population of molecules, for use in isolating and/or expanding cells with specificity to ECD3 of the US28 protein of HCMV (preferably with strain-agnostic specificity), and optionally wherein the cells are T cells and/or B cells, wherein the isolated population of molecules comprises, consists essentially of, or consists of, one or more peptides or polypeptides selected from either or both of the thirteenth and/or fourteenth aspects of the present invention, a combination of at least two (e.g. three) distinct peptides and/or polypeptides according to the fifteenth aspect of the present invention, one or more fusion proteins according to the sixteenth aspect of the present invention, and/or a combination of at least two (e.g. three) distinct fusion proteins according to the seventeenth aspect of the present invention, one or more conjugates according to eighteenth aspect of the present invention, and/or a combination of at least two (e.g. three) distinct conjugates according the nineteenth aspect of the present invention.

The present invention further provides a method of isolating and/or expanding cells capable of specifically targeting ECD1 (and optionally ECD3) of the US28 protein of HCMV (preferably with strain-agnostic specificity), wherein the method comprises providing a source of cells, and using an isolated population of molecules as defined in the preceding paragraph, in a method of selecting cells with specificity to ECD1 and/or ECD3 of the US28 protein of HCMV (and preferably selecting for strain-agnostic specificity), and expanding the selected cells, and optionally wherein the source of cells is selected from the group consisting of:
i. cells from a subject that is to be treated and/or in whom an HCMV infection or disease or condition associated therewith is to be combatted, in accordance with any of the various aspects of the present invention;
ii. cells that are HLA-matched to a subject that is to be treated and/or in whom an HCMV infection or disease or condition associated therewith is to be combatted, in accordance with any of the various aspects of the present invention;
iii. peripheral blood leukocytes or PBMCs; and
iv. T cells or B cells.

Said selected cells may optionally be:
(a) T cells with a T cell receptor (TcR) specific to ECD1 (and optionally ECD3) of the US28 protein of HCMV (preferably with strain-agnostic specificity);
(b) B cells with surface-bound antibody specific to ECD1 (and optionally ECD3) of the US28 protein of HCMV (preferably with strain-agnostic specificity); and/or
(c) B cells that secrete antibody specific to ECD1 (and optionally ECD3) of the US28 protein of HCMV (preferably with strain-agnostic specificity).

A twenty-sixth aspect of the present invention provides a cell that is exposed to, and/or comprising, one or more peptide or polypeptide selected from either or both of the thirteenth and/or fourteenth aspects of the present invention, a combination of at least two (e.g. three) distinct peptides and/or polypeptides according to the fifteenth aspect of the present invention, a fusion protein according to the sixteenth aspect of the present invention, and/or a combination of at least two (e.g. three) distinct fusion proteins according to the seventeenth aspect of the present invention, a conjugate according to eighteenth aspect of the present invention, a combination of at least two (e.g. three) distinct conjugates according the nineteenth aspect of the present invention, a nucleic acid molecule, or combination of multiple distinct nucleic acid molecules, according to the twenty-third aspect of the present invention, and/or a vector according to the twenty-fourth aspect of the present invention.

For example, the cell (or population of cells) may be antigen presenting cells (APCs). Optionally, the APCs are selected from the group consisting of dendritic cells (DCs), macrophages, Langerhans cells and B cells.

Optionally the cell (or population of cells) may be, or have been, pulsed with one or more peptide or polypeptide selected from either or both of the thirteenth and/or fourteenth aspects of the present invention, a combination of at least two (e.g. three) distinct peptides and/or polypeptides according to the fifteenth aspect of the present invention, a fusion protein according to the sixteenth aspect of the present invention, and/or a combination of at least two (e.g. three) distinct fusion proteins according to the seventeenth aspect of the present invention, a conjugate according to eighteenth aspect of the present invention, and/or a combination of at least two (e.g. three) distinct conjugates according the nineteenth aspect of the present invention. Additionally, or alternatively, the cell (or population of cells) may express, or have expressed the nucleic acid molecule, or combination of multiple distinct nucleic acid molecules, according to the twenty-third aspect of the present invention, and/or a vector according to the twenty-fourth aspect of the present invention, and thereby comprise peptides or polypeptide sequences encoded by said one or more nucleic acids.

Optionally the cells, such as APCs (e.g. DCs) may be from a subject that is to be treated and/or in whom an HCMV infection or disease or condition associated therewith is to be combatted, in accordance with any of the various aspects of the present invention.

Optionally the DCs may be monocyte-derived DCs (MoDCs), and for example the monocytes from which the DCs are derived may be monocytes from a subject that is to be treated and/or in whom an HCMV infection or disease or condition associated therewith is to be combatted, in accordance with any of the various aspects of the present invention.

Optionally, DCs are from a donor that is HLA-matched to a subject that is to be treated and/or in whom an HCMV infection or disease or condition associated therewith is to be combatted, in accordance with any of the various aspects of the present invention.

Optionally, the DCs are MoDCs and the monocytes from which the DCs are derived are monocytes from a donor that is HLA-matched to a subject to a subject that is to be treated and/or in whom an HCMV infection or disease or condition associated therewith is to be combatted, in accordance with any of the various aspects of the present invention.

A twenty-seventh aspect of the present invention provides a method of isolating and/or enriching cells comprising a T cell receptor (TCR) with specificity to an epitope in ECD1 (and optionally ECD3) of US28 (e.g. naturally occurring T cells, or recombinant cells expressing a CAR according to the first aspect of the present invention, for example CAR T-cells, CAR NK-cells and/or CAR-macrophages), wherein the method comprises the step of using one or more agents to isolate and/or enrich cells with binding specificity to one or all of the sequences of SEQ ID Nos: 6, 7 and/or 177,

wherein the one or more agents is or are selected from the group consisting of a peptide or polypeptide according to either or both of the thirteenth and/or fourteenth aspects of the present invention, a combination of at least two (e.g. three) distinct peptides and/or polypeptides according to the fifteenth aspect of the present invention, a fusion protein according to the sixteenth aspect of the present invention, and/or a combination of at least two (e.g. three) distinct fusion proteins according to the seventeenth aspect of the present invention, a conjugate according to eighteenth aspect of the present invention, and/or a combination of at least two (e.g. three) distinct conjugates according the nineteenth aspect of the present invention.

In one embodiment of the method of the twenty-seventh aspect of the present invention, the sequences can be formulated as an MHC tetramer.

MHC tetramers have emerged as an important tool for characterization of the specificity and phenotype of T cell immune response, and thus also for cells recombinantly expressing a T cell receptor (TCR), useful in a large variety of disease and vaccine studies. Peptide-MHC multimers typically display an antigenic peptide in the MHC binding groove, functioning as a surrogate for recognition events that occur as part of the T cell receptor interaction with antigen-presenting cell. The most prevalent form of multimer in use today consists of biotin-labeled pMHC displayed on streptavidin molecules, forming tetravalent complexes; so the common use of the term “tetramers” for this detection method. Since first description, MHC tetramers have become widely used for quantitation of antigen-specific T cell response. When coupled with methods that predict peptide binding to MHC molecules, tetramer analysis can be extremely useful for identifying T cell receptor epitopes. Specifically, the application of Class I tetramers to study self antigens has also extensively developed in studies of tumor antigens.

In certain embodiments, in accordance with the twenty-seventh aspect of the present invention, said MHC tetramer may for example be a Class I MHC tetramner for antigen-specific CD8⁺ T cells detection, a Class II MHC tetramer for antigen-specific CD4⁺ T cells detection, or a fluorophore-labelled tetramer for flow cytometry or fluorescence microscopy.

Optionally, the T-cell may be selected from the group consisting of CD8⁺ T cells, CD4+ T cells, effector T cells, helper T cells, memory T cells, cytotoxic T lymphocytes (CTLs), EBV-specific T cell receptor (TCR) or γδ-T cell subtypes.

A twenty-eighth aspect of the present invention provides an MHC tetramner comprising a peptide or polypeptide according a peptide or polypeptide according to either or both of the thirteenth and/or fourteenth aspects of the present invention, a combination of at least two (e.g. three) distinct peptides and/or polypeptides according to the fifteenth aspect of the present invention, optionally wherein the or each peptide comprises or corresponds to SEQ ID NO: 177, SEQ ID NO: 6 or SEQ ID NO: 7, or an immunogenic fragment of either or each, for example wherein the MHC tetramer is a Class I MHC tetramer for antigen-specific CD8⁺ T cells detection, a Class II MHC tetramer for antigen-specific CD4⁺ T cells detection, a fluorophore-labelled tetramer for flow cytometry or fluorescence microscopy. The MHC tetramer may further be used in isolating and/or enriching cells comprising a T cell receptor (TCR) with specificity to an epitope in ECD1 and/or ECD3 of US28, for example, a T-cell selected from the group consisting of CD8⁺ T cells, CD4⁺ T cells, effector T cells, helper T cells, memory T cells, cytotoxic T lymphocytes (CTLs), EBV-specific T cell receptor (TCR) or γδ-T cell subtypes and/or a recombinant cell expressing a CAR according to the first aspect of the present invention, for example a CAR T-cell, CAR NK-cell and/or CAR-macrophage.

### J. Formulations

Products according to the various aspects of the present invention may be formulated for further uses. For example, they may be formulated as pharmaceutically or veterinarially acceptable formulations.

The pharmaceutically or veterinarially acceptable formulations may be prepared in a manner known in the art that is sufficiently storage stable and suitable for administration to humans and animals. In one option, they may be presented in the form of a solution, such as a sterile solution. In another option, in particular in respect of formulations of non-cellular products, the pharmaceutically or veterinarially acceptable formulations may be lyophilised, e.g. through freeze drying, spray drying, spray cooling, or through use of particle formation from supercritical particle formation.

Such non-cellular products can include, without limitation, and one or more of: a binding molecule of the first or seventh aspects of the present invention; a nucleic acid molecule, or combination of multiple distinct nucleic acid molecules, according to the second aspect of the present invention; a vector, or combination of multiple distinct vectors according to the third aspect of the present invention; a conjugate according to the eight aspect of the present invention; an isolated conjugate according to the tenth aspect of the present invention; a non-cellular agent according to the various non-cellular options of agent provided for by the twelfth aspect of the present invention; one or more peptide or polypeptide selected from either or both of the thirteenth and/or fourteenth aspects of the present invention, a combination of at least two (e.g. three) distinct peptides and/or polypeptides according to the fifteenth aspect of the present invention, a fusion protein according to the sixteenth aspect of the present invention, and/or a combination of at least two (e.g. three) distinct fusion proteins according to the seventeenth aspect of the present invention, a conjugate according to eighteenth aspect of the present invention, a combination of at least two (e.g. three) distinct conjugates according the nineteenth aspect of the present invention, a nucleic acid molecule, or combination of multiple distinct nucleic acid molecules, according to the twenty-third aspect of the present invention, and/or a vector according to the twenty-fourth aspect of the present invention; an MHC tetramer according to the twenty-eighth aspect of the present invention; a non-cellular vaccine composition according to the various non-cellular options provided for by the twenty-ninth aspect of the present invention provides.

By “pharmaceutically acceptable” we mean a non-toxic material that does not decrease the effectiveness of the one or more formulated products according to the various aspects of the present invention, including one or more pharmaceutically acceptable buffers, carriers and/or excipients. Such pharmaceutically acceptable buffers, carriers or excipients are well-known in the art (see Remington's Pharmaceutical Sciences, 18′ edition, A. R Gennaro, Ed., Mack Publishing Company (1990) and handbook of Pharmaceutical Excipients, 3^{rd} edition, A. Kibbe, Ed., Pharmaceutical Press (2000)).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the polypeptide in the pharmaceutical preparation. The diluent may, for example, be one or more of saline, water, polyethylene glycol, propylene glycol, ethanol or oils (such as safflower oil, corn oil, peanut oil, cottonseed oil or sesame oil).

Optionally, a pharmaceutically or veterinarially acceptable formulation may comprise an adjuvant. The term “adjuvant” is intended to mean any compound added to the formulation to increase the biological effect of the one or more products of the invention within the formulation. The adjuvant may be one or more of zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and acetates of different acyl composition. The adjuvant may also be cationic polymers such as cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, glucose, sucrose, mannitol, and cyclodextrines, which are added to the composition, e.g. for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polysulphonate, polyethyleneglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the formulation, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

In an embodiment of the medical uses of the invention, the formulation may also comprise at least one further therapeutic agent (e.g. anti-cancer agent and/or anti-angiogenesis compound, examples of which are discussed above in section H of this application).

In one embodiment, the pharmaceutical compositions of the invention may be in the form of a liposome, in which the one or more products according to the various aspects of the present invention (hereinafter referred to as the “active agent(s)”) may be formulated is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations can be found in for example U.S. Pat. No. 4,235,871.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microspheres. Preparations of such microspheres can be found in U.S. Pat. No. 5,851,451 and in EP 0 213 303.

In a further embodiment, the pharmaceutical compositions of the invention are provided in the form of polymer gels, where polymers such as starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polyvinyl imidazole, polysulphonate, polyethyleneglycol/polyethylene oxide, polyethyleneoxide/polypropylene oxide copolymers, polyvinylalcohol/polyvinylacetate of different degree of hydrolysis, and polyvinylpyrrolidone are used for thickening of the solution containing the agent. The polymers may also comprise gelatin or collagen.

Alternatively, the active agent(s) may simply be dissolved in saline, water, polyethylene glycol, propylene glycol, ethanol or oils (such as safflower oil, corn oil, peanut oil, cottonseed oil or sesame oil), tragacanth gum, and/or various buffers.

It will be appreciated that the pharmaceutical compositions of the invention may include ions and a defined pH for potentiation of action of the active polypeptide. Additionally, the compositions may be subjected to conventional pharmaceutical operations such as sterilisation and/or may contain conventional adjuvants such as preservatives, stabilisers, wetting agents, emulsifiers, buffers, fillers, etc.

Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents.

In an alternative preferred embodiment, the pharmaceutical composition is suitable for topical administration to a patient.

Preferably, the formulation is a unit dosage containing a daily dose or unit, daily sub-dose or an appropriate fraction thereof, of the active ingredient.

It will be appreciated that in the below described routes of administration, the skilled person would know which active agents are applicable for any given route of administration.

The active agent(s) may be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy the active agent(s) will generally be administered in admixture with a suitable pharmaceutical excipient, diluent or carrier selected with regard to the intended route of administration and standard pharmaceutical practice.

For example, the active agent(s) may be administered orally, buccally or sublingually in the form of tablets, capsules, ovules, elixirs, solutions or suspensions, which may contain flavouring or colouring agents, for immediate-, delayed- or controlled-release applications. The active agent(s) may also be administered via intracavernosal injection.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPNC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The active agent(s) can also be administered parenterally, for example, intravenously, intra-arterially, intraperitoneally, intrathecally, intraventricularly, intrasternally, intracranially, intramuscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The formulations may be presented as unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of an active agent will usually be from 1 to 1,000 mg per adult (i.e. from about 0.015 to 15 mg/kg), administered in single or divided doses.

Thus, for example, the tablets or capsules of the active agent(s) may contain from 1 mg to 1,000 mg of active agent for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited, and such are within the scope of this invention.

The active agent(s) can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of an active agent and a suitable powder base such as lactose or starch. Such formulations may be particularly useful for treating solid tumours of the lung, such as, for example, small cell lung carcinoma, non-small cell lung carcinoma, pleuropulmonary blastoma or carcinoid tumour.

Aerosol or dry powder formulations are preferably arranged so that each metered dose or “puff” contains an effective amount (e.g. at least 1 mg) of the active agent(s) for delivery to the subject. It will be appreciated that the overall daily dose with an aerosol will vary from patient to patient, and may be administered in a single close or, more usually, in divided doses throughout the day.

Alternatively, the active agent(s) can be administered in the form of a suppository or pessary, particularly for treating or targeting colon, rectal or prostate tumours.

The active agent(s) may also be administered by the ocular route. For ophthalmic use, the active agent(s) can be formulated as, e.g. micronised suspensions in isotonic, pH adjusted, sterile saline, or, preferably, as solutions in isotonic, pH adjusted, sterile saline, optionally in combination with a preservative such as a benzylalkonium chloride. Alternatively, the active agent(s) may be formulated in an ointment such as petrolatum. Such formulations may be particularly useful for treating solid tumours of the eye, such as retinoblastoma, medulloepithelioma, uveal melanoma, rhabdornyosarcoma, intraocular lymphoma, or orbital lymphoma.

The active agent(s) may be applied topically in the form of a lotion, solution, cream, ointment or dusting powder, or may be transdermally administered, for example, by the use of a skin patch. For application topically to the skin, the active agent(s) can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water. Such formulations may be particularly useful for treating solid tumours of the skin, such as, for example, basal cell cancer, squamous cell cancer or melanoma.

For skin cancers, the active agent(s) can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with inhibitor or can simply act as “bullets” that generate pores in the skin through which the agent, antibody or compound can enter.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier. Such formulations may be particularly useful for treating solid tumours of the mouth and throat.

In an embodiment, when the active agent(s) comprise, consist essentially of, or consist of polypeptide(s), then the active agent(s) may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The active agent(s) can be administered by a surgically implanted device that releases the active agent(s) directly to the required site, for example, into the eye to treat ocular tumours. Such direct application to the site of disease achieves effective therapy without significant systemic side-effects.

An alternative method for delivery of the active agent(s) that comprise, consist essentially of, or consist of polypeptide(s), such as antibodies and CARs, is the ReGel injectable system that is thermo-sensitive. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Polypeptide active agent(s) can also be delivered orally. The process employs a natural process for oral uptake of vitamin B₁₂ in the body to co-deliver proteins and peptides. By riding the vitamin B₁₂ uptake system, the protein or peptide active agent(s) can move through the intestinal wall. Complexes are synthesised between vitamin B₁₂ analogues and the drug that retain both significant affinity for intrinsic factor (IF) in the vitamin B₁₂ portion of the complex and significant bioactivity of the drug portion of the complex.

Nucleic acid active agent(s) may be administered as a suitable genetic construct as described herein and delivered to a subject for expression. Typically, the nucleic acid in the genetic construct is operatively linked to a promoter which can express the compound in the cell. The genetic constructs of the invention can be prepared using methods well known in the art, for example in Sambrook et al. (2012) MOLECULAR CLONING: A LABORATORY MANUAL, volumes 1-4, Cold Spring Harbor Press, NY.

Preferably, the genetic construct is adapted for delivery to a human cell. Means and methods of introducing a genetic construct into a cell are known in the art, and include the use of immunoliposomes, liposomes, viral vectors (including vaccinia, modified vaccinia, lentivirus, parvovirus, retroviruses, adenovirus and adeno-associated viral (AAV) vectors), and by direct delivery of DNA, e.g. using a gene-gun and electroporation. Furthermore, methods of delivering polynucleotides to a target tissue of a patient for treatment are also well known in the art. In an alternative method, a high-efficiency nucleic acid delivery system that uses receptor-mediated endocytosis to carry DNA macromolecules into cells is employed. This is accomplished by conjugating the iron-transport protein transferrin to polycations that bind nucleic acids. High-efficiency receptor-mediated delivery of the DNA constructs or other genetic constructs of the invention using the endosome-disruption activity of defective or chemically inactivated adenovirus particles produced by the methods of Cotten et al. (1992) Proc. Natl. Acad. Sci. USA 89, 6094-6098 may also be used. It will be appreciated that “naked DNA” and DNA complexed with cationic and neutral lipids may also be useful in introducing the DNA of the invention into cells of the individual to be treated. Non-viral approaches to gene therapy are described in Ledley (1995, Human Gene Therapy 6, 1129-1144).

Although for cancer/tumours of specific tissues it may be useful to use tissue-specific promoters in the vectors encoding a nucleic acid molecule of the present invention, this is not essential, as the risk of expression of the nucleic acid sequence in the body at locations other than the cancer/tumour would be expected to be tolerable in comparison with the therapeutic benefit to a patient suffering from a cancer/tumour.

In an embodiment, the pharmaceutical compositions or formulations of the invention are for parenteral administration, more particularly for intravenous administration. In a preferred embodiment, the pharmaceutical composition is suitable for intravenous administration to a patient, for example by injection. In other embodiments, intra-thecal administration may be used, for example to target the brain and/or the nervous system.

### K. Vaccines

A twenty-ninth aspect of the present invention provides a vaccine composition suitable for use in vaccinating against, reducing the risk of, preventing, or combating an HCMV infection and/or a disease or condition associated with human cytomegalovirus (HCMV). Said an HCMV infection and/or a disease or condition associated with human cytomegalovirus (HCMV) may be as defined in section G of this application.

The vaccine may comprise the components of an active vaccine and/or a passive vaccine. An active vaccine component is designed to provoke active immunity; whereas a passive vaccine component is designed to provide passive immunity.

An active vaccine according to the twenty-ninth aspect of the present invention may, when administered to a subject, induce the subject's immune system to generate an immune response directed to an epitope present within ECD1 and/or ECD3 of a US28 protein of HCMV, preferably a strain agnostic immune response.

A passive vaccine according to the twenty-ninth aspect of the present invention may be a composition that, when administered to a subject, provides an immune system capability that is directed to an epitope present within ECD1 and/or ECD3 of a US28 protein of HCMV, preferably a strain agnostic immune system capability.

In some embodiments, the vaccine composition of the twenty-ninth aspect of the present invention triggers and/or provides an immune response:
(a) to one or more epitopes present entirely within ECD1 of the US28 protein of HCMV (for example, to an epitope present in one or more of the immunogenic fragments of SEQ ID Nos: 177, as described in accordance with the thirteenth aspect of the present invention);
(b) to one or more linear epitopes within ECD1 of the US28 protein;
(c) has to one or more epitopes present entirely within extracellular domain 3 (ECD3) of the US28 protein of HCMV (for example, to an epitope present in one or more of the immunogenic fragments of SEQ ID Nos: 6 or 7, as described in accordance with the fourteenth aspect of the present invention);
(d) to one or more linear epitopes within ECD3 of the US28 protein;
(e) to one or more epitopes within ECD3 of a US28 protein of HCMV that is an epitope, or are epitopes, present in identical form in both the 4D-variant strains and 4N-variant strains of HCMV, wherein the 4D-variant strain of HCMV encodes a US28 protein comprising an ECD3 having the sequence of TKKDNQCMTDYDYLEVS (SEQ ID NO: 7) and wherein the 4N-variant strain of HCMV encodes a US28 protein comprising an ECD3 having the sequence of TKKNNQCMTDYDYLEVS (SEQ ID NO: 6); and/or
(f) wherein the immune response that is triggered or provided by the vaccine is HCMV strain agnostic to the 4D-variant strains and 4N-variant strains of HCMV, and triggers and/or provides an immune response that is directed to one or more of the 4D-variant HCMV strains selected from Towne, VR1814, TB40/E, Merlin, JP, Ad169, VHL/E, AF1, BL and DAVIS and is also directed to one or more of the 4N-variant HCMV strains selected from Toledo, TR and DB.

Said vaccine composition may be a passive vaccine, and/or optionally comprise: (a) one or more binding molecules according to the first aspect of the present invention, (b) one or more functional fragments of said one or more binding molecules as defined by the first aspect of the present invention, (c) one or more isolated binding molecules according to the seventh aspect of the present invention, (d) one or more nucleic acid molecules, or combination of multiple distinct nucleic acid molecules, according to the second aspect of the present invention, (e) one or more vectors according to the third aspect of the present invention, (f) one or more cells according to the fourth aspect of the present invention, (g) one or more conjugates according to the eighth aspect of the present invention, and/or (h) one or more isolated conjugates according to the tenth aspect of the present invention.

Alternatively, said vaccine composition may be an active vaccine, and/or optionally comprise:
(a) one or more peptides or polypeptides according to either or both of the thirteenth and/or fourteenth aspects of the present invention, a combination of at least two (e.g. three) distinct peptides and/or polypeptides according to the fifteenth aspect of the present invention, a fusion protein according to sixteenth aspect of the present invention, a combination of at least two (e.g. three) distinct fusion proteins according to the seventeenth aspect of the present invention, a conjugate according to the eighteenth aspect of the present invention, and/or a combination of at least two (e.g. three) distinct conjugates according to the nineteenth aspect of the present invention;
(b) one or more nucleic acid molecules, or combination of multiple distinct nucleic acid molecules, according to the twenty-third aspect of the present invention, and/or the vector according to the twenty-fourth aspect of the present invention; and/or
(c) a cell, such as an antigen-presenting cell (e.g. a dendritic cell), or a homogeneous or heterogeneous population of said cells, wherein the or each of said cells is loaded with one or more of the following: a peptide or polypeptide according to either or both of the thirteenth and/or fourteenth aspects of the present invention, a combination of at least two (e.g. three) distinct peptides and/or polypeptides according to the fifteenth aspect of the present invention, a fusion protein according to sixteenth aspect of the present invention, a combination of at least two (e.g. three) distinct fusion proteins according to the seventeenth aspect of the present invention, a conjugate according to the eighteenth aspect of the present invention, a combination of at least two (e.g. three) distinct conjugates according to the nineteenth aspect of the present invention, one or more nucleic acid molecules, or combination of multiple distinct nucleic acid molecules, according to the twenty-third aspect of the present invention, and/or the vector according to the twenty-fourth aspect of the present invention.

A thirtieth aspect of the present invention provides a method of vaccinating against, reducing the risk of, preventing, and/or combating a disease or condition associated with HCMV, the method comprising administering to a subject a vaccine according to the twenty-ninth aspect of the present invention.

The thirtieth aspect of the present invention provides a vaccine according to the twenty-ninth aspect of the present invention for use in vaccinating against, reducing the risk of, preventing, and/or combating a disease or condition associated with HCMV in a subject.

The thirtieth aspect of the present invention provides for the use of a vaccine according to the twenty-ninth aspect of the present invention in the manufacture of a medicament for vaccinating against, reducing the risk of, preventing, and/or combating a disease or condition associated with HCMV.

Said disease or condition associated with HCMV may, for example, be a disease or condition associated with HCMV as disclosed above in the context of the eleventh aspect of the present invention. Optionally, the disease or condition is a latent HCMV infection, or is a disease or condition associated with a latent HCMV infection.

In some embodiments, the method of vaccinating against, reducing the risk of, preventing, and/or combating a disease or condition associated with HCMV may comprise administering the vaccine to the subject only once.

In other embodiments, the method of vaccinating against, reducing the risk of, preventing, and/or combating a disease or condition associated with HCMV may comprise administering the vaccine to the subject vaccine twice or multiple times. For example, in the embodiment in which the vaccine is an active vaccine, it may be appropriate to separately administer a primary dose, and a subsequent booster dose, to the subject.

### L. Diagnostics and Other Modes of Detection and Assessment

As discussed in the Summary of the Invention section of this application, a thirty-first aspect of the present invention provides a method of assessing one or more biological conditions and/or biological characteristics of a subject and/or of ex vivo biological material.

Said method makes use of the binding characteristics of binding molecules or conjugate thereof, according to the present invention, to bind with high specificity to the ECD1 and/or ECD3 of US28 protein, and preferably to bind with high specificity yet without discriminating between different strains of HCMV.

The method comprises: (a) contacting the subject and/or the ex vivo biological material with a binding molecule as defined by the first aspect of the present invention, or a conjugate as defined by the eighth or tenth aspect of the present invention; and (b) making an assessment of the subject and/or the ex vivo biological material based on a direct and/or indirect measurement of the binding of the binding molecule or conjugate to the subject and/or the ex vivo biological material.

Any suitable method may be used for the assessment. For example, and without limitation, the method may comprise an ELISA method, and optionally the method is performed on ex vivo biological material, such as one or more body fluids (e.g. blood, saliva, urine).

In an alternative example, the method comprises a flow cytometry method, and optionally the method is a method for assessing an ex vivo blood sample and/or ex vivo bone marrow sample from a subject.

In a further alternative embodiment, the method comprises the use of a conjugate as defined by the eighth or tenth aspect of the present invention, wherein the conjugate comprises a detectable moiety, such as radioactive moiety, and the method comprises detection of the detectable moiety in the subject and/or the ex vivo biological material, for example, wherein the method is a method of immune-positron emitting (PET) imaging.

In a further alternative embodiment, the method is a method of immunohistochemistry (IHC), for example IHC that is performed on a sample of ex vivo biological material. Said sample may, for example, be a histological specimen from a biological material obtained from a subject or other biological source of interest. The skilled person is well aware of numerous techniques well known in the art for the preparation of a histological specimens from a biological material. Any suitable means for the preparation of a histological specimen can be used in the context of the present invention.

In one embodiment, the method of the thirty-first aspect of the present invention is performed on a subject, or on ex vivo biological material obtained from the subject, for the purposes of generating data (such as an image or other measurable data) that is suitable for making a diagnostic or prognostic assessment of an HCMV infection and/or a disease or condition associated with HCMV in the subject.

Said HCMV infection and/or a disease or condition associated with HCMV for which a diagnostic or prognostic assessment can be made (or data pertaining thereto can be generated) by a method of the thirty-first aspect of the present invention may, for example, be as defined in section G of this application.

The subject for which a diagnostic or prognostic assessment can be made (or data pertaining thereto can be generated) by a method of the thirty-first aspect of the present invention may, for example, be any subject described in section G of this application.

The disease or condition associated with HCMV, for which a diagnostic or prognostic assessment can be made (or data pertaining thereto can be generated) by a method of the thirty-first aspect of the present invention, may be an HCMV infection or be associated with an HCMV infection.

The HCMV infection may, in one embodiment, be a single strain infection.

Optionally, in an alternative embodiment, the HCMV infection comprises a multi-strain HCMV infection, wherein the multi-strain HCMV infection comprises infection with more than one different strain of HCMV, for example two or more HCMV strains that encode different US28 protein encodes sequences. Said two or more strains may encode US28 proteins that differ in one or more of the extracellular regions, such as in the N-terminal (ECD1) regional, the first extracellular loop (ECD2) region, the second extracellular loop (ECD3) region, and/or the third extracellular loop (ECD4) region. In one embodiment of interest, the two or more HCMV strains in a multi-strain HCMV infection each encode a US2S protein that differs from the other at least in one or more positions of the N-terminal (ECD1) region; for example they may differ at 1, 2, 3, 4, 5, 6, 8, 9, 10 or more amino acid positions in the N-terminal (ECD1) region. The differences in one or more positions of the N-terminal (ECD1) region may be outside of positions 26-37; for example the one or more positions that differ may be within positions 1-25 (such as any one or more of positions 8, 15, 16, 18, 19, 24, 25, including but not limited to any one or more of the sequence variations listed in Table 3 of the present application), for example within one or more of positions 1-10, 16, 18 and/or 19, wherein the positions are with respect to the numbering of positions in SEQ ID NO: 5. Additionally, or alternatively, in another embodiment of interest, the two or more HCMV strains in a multi-strain HCMV infection each encode a US28 protein that differs from the other at one or more positions of the second extracellular loop (ECD3) region, for example one or more of the HCMV strains in a multi-strain HCMV infection may encode a US28 protein that encodes the 4N-variant of ECD3, and one or more of the other HCMV strains in a multi-strain HCMV infection may encode a US28 protein that encodes the 4D-variant of ECD3.

The disease or condition associated with HCMV, for hash a diagnostic or prognostic assessment can be made (or data pertaining thereto can be generated) by a method of the thirty-first aspect of the present invention, may be a latent HCMV infection (for example, a single or multi-strain latent HCMV infection) or be associated with a latent HCMV infection (optionally a multi-strain latent HCMV infection).

The disease or condition associated with HCMV, for hash a diagnostic or prognostic assessment can be made (or data pertaining thereto can be generated) by a method of the thirty-first aspect of the present invention, may be a lytic HCMV infection (optionally a multi-strain lytic HCMV infection) or be associated with a lytic HCMV infection (optionally a multi-strain lytic HCMV infection).

The disease or condition associated with HCMV, for hash a diagnostic or prognostic assessment can be made (or data pertaining thereto can be generated) by a method of the thirty-first aspect of the present invention, may be a congenital HCMV infection (for example, a single or multi-strain infection), such as a latent congenital single or multi-strain HCMV infection or a lytic congenital single or multi-strain HCMV infection. Accordingly, the subject of such an assessment may be the subject in respect of whom the disease or condition is to be assessed, such an infant (such as a neonate), a foetus or embryo, or the mother (or prospective mother of the subject), such as a breast-feeding mother of the subject, a female pregnant with the subject, or a female that is to be assessed prior to conception of the individual.

The disease or condition associated with HCMV, for which a diagnostic or prognostic assessment can be made (or data pertaining thereto can be generated) by a method of the thirty-first aspect of the present invention, may be cancer, for example HCMV-infected cancer (optionally a single-strain, or multi-strain, HCMV infected cancer), such as latent HCMV-infected cancer (optionally a single-strain, or multi-strain, latent HCMV infected cancer).

The disease or condition associated with HCMV, for which a diagnostic or prognostic assessment can be made (or data pertaining thereto can be generated) by a method of the thirty-first aspect of the present invention, may be an epithelial cancer; optionally wherein the epithelial cancer is breast cancer; for example, wherein the breast cancer is triple negative breast cancer (TNBC), or a HER2-positive breast cancer (as described herein). Said forms of epithelial cancer may optionally be a single-strain, or multi-strain, form of HCMV infected epithelial cancer, for example a latent HCMV-infected form of epithelial cancer (optionally a single-strain, or multi-strain, latent HCMV infected cancer).

The disease or condition associated with HCMV, for hash a diagnostic or prognostic assessment can be made (or data pertaining thereto can be generated) by a method of the thirty-first aspect of the present invention, may be a metastasising and/or aggressive form of cancer. Said forms of metastasising and/or aggressive cancer may optionally be a single-strain, or multi-strain, form of HCMV infected metastasising and/or aggressive cancer, for example a latent HCMV-infected form of metastasising and/or aggressive cancer (optionally a single-strain, or multi-strain, latent HCMV infected cancer).

The disease or condition associated with HCMV, for which a diagnostic or prognostic assessment can be made (or data pertaining thereto can be generated) by a method of the thirty-first aspect of the present invention may be glioblastoma.

The disease or condition associated with HCMV, for which a diagnostic or prognostic assessment can be made (or data pertaining thereto can be generated) by a method of the thirty-first aspect of the present invention, may be made in a subject, or ex vivo biological material obtain from a subject, that has, has been diagnosed has having or possessing, or is suspect of having, HCMV-infected cancer cells, such as latent HCMV-infected cancer cells.

The disease or condition associated with HCMV, for hash a diagnostic or prognostic assessment can be made (or data pertaining thereto can be generated) by a method of the thirty-first aspect of the present invention, may be, or intended to be, the recipient of a cellular material, such as the donation of a cellular product. Said cellular product may, for example, comprise, consist essentially of, or consist of, living ex vivo cellular material selected from the group that includes: one or more types of ex vivo cells; one or more types of ex vivo cell cultures; one or more types of ex vivo tissues; one or more types of ex vivo tissue cultures; one or more types of ex vivo organs; and/or one or more types of ex vivo organ cultures. Optionally, the cellular product may be derived, directly or indirectly, from a living donor.

The disease or condition associated with HCMV, for which a diagnostic or prognostic assessment can be made (or data pertaining thereto can be generated) by a method of the thirty-first aspect of the present invention, may be assessed in a subject that is, or is intended to be the donor of a cellular material, such as the donor of a cellular product; or in ex vivo biological material from said donor. Said cellular product may be comprise, consist essentially of, or consist of any one or more of cells, tissue or an organ from said donor.

The disease or condition associated with HCMV, for which a diagnostic or prognostic assessment can be made (or data pertaining thereto can be generated) by a method of the thirty-first aspect of the present invention, may be conducted on biological material, such as ex vivo or in vitro cellular material, such as biological material that is suitable for and/or to be used for administration to a recipient subject. Said ex vivo cellular product may, for example, comprise, consist essentially of, or consist of, living ex vivo cellular material selected from the group that includes: one or more types of ex vivo cells; one or more types of ex vivo cell cultures; one or more types of ex vivo tissues; one or more types of ex vivo tissue cultures; one or more types of ex vivo organs; and/or one or more types of ex vivo organ cultures. Optionally, the cellular product may be derived, directly or indirectly, from a living donor.

Also provided herein is a kit, such as a diagnostic kit, comprising one or more components discussed above in this section, for example when presented within said kit in a form suitable for use (or, capable of being converted into a form suitable for use, e.g. by reconstitution or other simple manipulation of a stored form) in the performance of the method of assessing one or more biological conditions and/or biological characteristics of a subject and/or of ex vivo biological material in accordance with the thirty-first aspect of the present invention.

### M. Treatment of Ex Vivo Material

As noted above, some of the routes of HCMV transmission to a subject can be via contact with ex vivo biological material from an infected person, or from any other infected source. Examples include the implantation or transplantation, into said subject, of ex vivo biological material (such as organ, tissue, bone marrow or stem cell transplantation) or blood transfusions.

Accordingly, a thirty-second aspect of the present invention provides a method of combating a HCMV infection (such as a latent HCMV infection and/or a lytic HCMV infection and/or a multi-strain HCMV infection) in living ex vivo biological material, the method comprising contacting the living ex vivo biological material with any one or more agents selected from the group consisting of:
i. a binding molecule according to the first aspect of the present invention,
ii. a functional fragment of said binding molecule as defined by the first aspect of the present invention,
iii. an isolated binding molecule according to the seventh aspect of the present invention,
iv. a nucleic acid molecule, or combination of multiple distinct nucleic acid molecules, according to the second aspect of the present invention,
v. a vector according to the third aspect of the present invention,
vi. a cell according to the fifth aspect of the present invention,
vii. a conjugate according to the eighth aspect of the present invention, and
viii. an isolated conjugate according to the tenth aspect of the present invention.

The thirty-second aspect of the present invention also provides living ex vivo biological material that is obtained, or obtainable, by the method of this aspect.

In one embodiment of the method of the thirty-second aspect of the present invention, and/or the living ex vivo biological material obtained, or obtainable, thereby, the ex vivo living biological material comprises, consists essentially of, or consists of, living ex vivo biological material selected from the group that includes: one or more types of ex vivo cells; one or more types of ex vivo cell cultures; one or more types of ex vivo tissues; one or more types of ex vivo tissue cultures; one or more types of ex vivo organoids; one or more types of ex vivo organoid cultures; one or more types of ex vivo organs; and/or one or more types of ex vivo organ cultures. Exemplary embodiments include organ transplants, tissue transplants, bone marrow transplants, stem cell transplants, and or blood transfusions

A thirty-third aspect of the present invention provides a method of treating a subject in need thereof (that is, for example, a subject in need of a transplantation or implantation of said ex vivo living biological material), comprising administering ex vivo living biological material as defined by the thirty-second aspect of the present invention, to the subject.

For example, the method may be a method of transplantation of the ex vivo living biological material, such as an organ or tissue transplant. Said method can be used to prevent, or reduce the risk of, the transmission of an HCMV infection, or a disease or condition associated with an HCMV infection in the recipient of the transplant.

The subject to be treated in accordance with the thirty-third aspect of the present invention may, for example, be any subject as described in section G of this application.

The disease or condition associated with HCMV in accordance with the thirty-third aspect of the present invention may, for example, be any form of HCMV infection or disease or condition associated an HCMV infection as described in section G of this application.

### N. Methods of Screening for Binding Molecules

As described in the Examples of the present application, the applicant has developed a new approach to the identification of highly specific, strain agnostic, binding molecules against the HCMV US28 protein, by focusing on raising binding molecules to epitopes within the ECD1 and/or ECD3 region of HCMV US28. As reported in the examples of the present application, a number of ECD1-monoclonal antibodies generated by this approach were shown to bind well and specifically to a peptide representing a conserved epitope within ECD1, on US 28 overexpressing US28-CHO-A1 cells, and HCMV infected human lung tissue. Further, a number of ECD3-monoclonal antibodies generated by this approach were shown to bind well and specifically on both genetic variants of the US28 ECD1 and/or ECD3 peptides, thereby demonstrating both specificity and strain agnostic binding properties, and further demonstrated specific binding on US28 overexpressing US28-CHO-A1 cells, HCMV Ad169 infected MRC-5 cells, primary PBMCs from HCMV seropositive individuals, HCMV infected human lung tissue and several types of aggressive human tumors, such as oesophagus, gastric, rectum, liver, lung, pancreas, cervical cancers, malignant pheochromocytoma and locally advanced colon cancer, breast cancer and its metastasis and glioblastoma grade 4.

For ECD3, the applicant's approach made use of the identified single site of amino acid variation in ECD3 of US28, to utilise a combination of peptides selected to raise an immune response, and to generate and isolate antibodies having a strain agnostic binding specificity to the ECD3 of US28. This approach can be used to generate and identify further such binding molecules having the favourable binding properties of the present invention.

Accordingly, a thirty-fourth aspect of the present invention provides a method of screening for a binding molecule having binding specificity and/or binding affinity to an epitope within ECD11 and/or ECD3 of a US28 protein of human cytomegalovirus (HCMV), the method comprising:
(a) providing one or more peptides corresponding an amino acid sequence present in ECD1 and/or ECD3 of the US28 protein,
(b) providing one or more candidate binding molecules;
(c) determining the binding specificity and/or binding affinity of one or more candidate binding molecules to the one or more peptides.

Optionally the, or each, of the one or more peptides provided in step (a) are provided in the form of one or more peptides or polypeptides according to either or both of the thirteenth and/or fourteenth aspects of the present invention, a combination of at least two (e.g. three) distinct peptides and/or polypeptides according to the fifteenth aspect of the present invention, a fusion protein according to sixteenth aspect of the present invention, a combination of at least two (e.g. three) distinct fusion proteins according to the seventeenth aspect of the present invention, a conjugate according to the eighteenth aspect of the present invention, a combination of at least two (e.g. three) distinct conjugates according to the nineteenth aspect of the present invention

Optionally, the, or each, of the one or more peptides comprise, consist essentially of, or consist of, the polypeptide sequence TDVLNQSKPVTL (SEQ ID NO: 177), TKKDNQCMTDYDYLEVS (SEQ ID NO: 7) and/or TKKNNQCMTDYDYLEVS (SEQ ID NO: 6), and/or an immunogenic fragment of either or each.

Optionally, the one or more candidate binding molecules can be antibodies and/or CARs, such as any one or more of:
(a) bivalent antibodies, such as IgG-scFv antibodies (for example, wherein a first binding domain is an intact IgG and a second binding domain is an scFv attached to the first binding domain at the N-terminus of a light chain and/or at the C-terminus of a light chain and/or at the N-terminus of a heavy chain and/or at the C-terminus of a heavy chain of the IgG, or vice versa);
(b) monovalent antibodies, such as a DuoBody® or ‘knob-in-hole’ bispecific antibody (for example, an scFv-KIH, scFv-KIH^{r}, a BiTE-KIH or a BiTE-KIH^{r};
(c) scFv₂-Fc antibodies;
(d) bispecific antibodies, such as bispecific T-cell engager (BiTE) antibodies;
(e) dual variable domain (DVD)-Ig antibodies;
(f) dual-affinity re-targeting (DART)-based antibodies (for example, DART₂-Fc or DART);
(g) trispecific antibodies, such as DNL-Fab₂ antibodies;
(h) scFv-HSA-scFv antibodies; and
(i) a chimeric antigen receptor (CAR).

The method of the thirty-fourth aspect of the present invention may comprise the step of selecting a candidate binding molecule based on the binding specificity and/or binding affinity to the one or more peptides. For example, the selected candidate binding molecule:
(a) may have binding specificity to an epitope present entirely within ECD1 of the US28 protein of HCMV;
(b) may have binding specificity to a linear epitope within ECD1 of the US28 protein;
(c) may have binding specificity to an epitope within ECD1 of a US28 protein of HCMV that is HCMV strain agnostic, for example, binding specificity to an epitope within ECD1 of a US28 protein of HCMV that is agnostic to two or more (such as all) of HCMV strains selected from the group consisting of DB, Towne, AD169, BL, DAVIS, JP, Merlin, PH, TB40/E, Toledo, VHL/E, TR and VR1814 (FIX);
(d) may have binding specificity to an epitope present entirely within extracellular domain 3 (ECD3) of the US28 protein of HCMV;
(e) may have binding specificity to a linear epitope within ECD3 of the US28 protein;
(f) may have binding specificity to an epitope within ECD3 of a US28 protein of HCMV that is HCMV strain agnostic, for example, binding specificity to an epitope within ECD3 of a US28 protein of HCMV that is agnostic to two or more (such as all) of HCMV strains selected from the group consisting of DB, Towne, AD169, BL, DAVIS, JP, Merlin, PH, TB40/E, Toledo, VHL/E, TR and VR1814 (FIX); and/or
(g) may have specificity to an epitope within ECD3 of the US28 protein of HCMV, irrespective of whether the ECD3 of the US28 protein comprises the sequence of TKKDNQCMTDYDYLEVS (SEQ ID NO: 7) or TKKNNQCMTDYDYLEVS (SEQ ID NO: 6).

The selected candidate binding molecule may have a binding specificity and/or binding affinity to the one or more peptides that is equivalent to the binding specificity and/or binding affinity to the same one or more peptides as demonstrated by an antibody that comprises a variable heavy chain (V_{H}) polypeptide that consists of the sequence of SEQ ID NO: 187, 211, 233, 12, 104, 122, 68 or 88 and a variable light chain (V_{L}) polypeptide that consists of the sequence of SEQ ID NO: 189, 213, 235, 18, 108, 126, 72 or 92. Said antibody may, for example, be the 4H3C3, 7B1F3, 2F5B11, 1D3, 1C10, 1A10, 1G9 or 1E8 antibody as further described herein.

Functional variants of one or more of said selected candidate binding molecule can be created by any of the various processes well known in the art. For example, the binding properties of a selected candidate binding molecule (e.g. a selected candidate binding molecule that comprises 1, 2, 3, 4, 5 or 6 CDRs) can be developed by a maturation process. For example, the binding affinity can be modified (increased, or reduced) by maturation steps; and/or the binding specificity can be modified (in general, increased) by maturation steps. It is to be understood that high, or increased, levels of binding affinity to US28 may not always be desirable, in particular if this comes at the expense of unacceptably high levels of binding affinity to off-targets, such as healthy human cells. Binding molecules, including but not limited to CARs and CAR-expressing cells, for example, may benefit from lower levels of binding affinity, but will generally always benefit from optimal levels of binding specificity. Examples of a maturation process are discussed above, in Section A of this application.

A thirty-fifth aspect of the present invention provides a method of producing a composition that comprises multiple copies of a binding molecule, said method comprising causing the reproduction of a selected candidate binding molecule that has been selected in accordance with the method of the thirty-fourth aspect of the present invention.

Said method may, for example, comprise providing a nucleic acid molecule, or combination of multiple distinct nucleic acid molecules, wherein the nucleic acid molecule comprises, or the combination of multiple distinct nucleic acid molecules collectively comprise, one or more nucleic acid sequences that, individually or in combination, encode a selected candidate binding molecule. Said method may further comprise expressing said nucleic acid molecule, or combination of multiple distinct nucleic acid molecules, for example in a recombinant cell or population of cells transformed with said nucleic molecule, or combination of multiple distinct nucleic acid molecules. Such expression can be used to produce further copies of the selected candidate molecule.

Optionally said selected candidate molecule is subsequently isolated and/or purified and/or formulated, for example in the form of a pharmaceutically acceptable composition.

Optionally, said selected candidate molecule is conjugated, for example in accordance with the discussion of conjugates as discussed in Section A of this application.

A thirty-sixth aspect of the present invention provides a method of assessing a selected candidate binding molecule that has been selected in accordance with the method of the thirty-fourth aspect of the present invention and/or produced in accordance with the method of the thirty-fifth aspect of the present invention, said method comprising and identifying the structure(s) within the selected candidate binding molecule that provides its binding characteristics (in particular, the binding specificity and/or binding affinity to an epitope within ECD1 (and optionally ECD3) of a US28 protein of human cytomegalovirus (HCMV)).

For example, the step of identifying the structure(s) within the selected candidate binding molecule that provides its binding characteristics may include identifying and/or sequencing the, or each, variable light and/or variable heavy region in a selected candidate binding molecule that is an antibody or CAR.

Additionally, or alternatively, the step of identifying the structure(s) within the selected candidate binding molecule that provides its binding characteristics may include identifying and/or sequencing the, or each, CDR region in a selected candidate binding molecule that is an antibody or CAR.

Methods of identifying variable light and/or variable heavy regions, and of identifying CDR sequences, are well known in the art, and can be employed by the skilled person using routine techniques.

For example, the heavy and light chain variable domain sequence (V_{H} and V_{L}) of an antibody may be determined from heavy and light chain cDNA, synthesized from the respective mRNA by techniques generally known to the art. The CDR regions may then be determined by an art known technique. For example, CDR regions may be determined using the Kabat method (Wu and Kabat, J. (1970) J. Exp. Med. 132, 211). The CDRs may be determined by structural analysis using X-ray crystallography or molecular modelling techniques. A composite CDR may be defined as containing all the residues in one CDR and all the residues in the corresponding hypervariable region. These composite CDRs along with certain select residues from the framework region are preferably identified as transferrable “antigen binding sites”. Other well-known methods of identifying CDR sequences, include the Chothia numbering scheme (for example, as described in Al-Lazikani et al., JMB, 1997, 273: 927-948) or the Martin (Enhanced Chothia) Numbering Scheme.

The CDRs of the binding molecules and polypeptides of the present invention were determined using the Kabat method. Accordingly, in some embodiments, 1, 2, 3, 4, 5 or 6 of the CDRs of a polypeptide or binding molecule of the present invention has/have been determined using the Kabat method. Furthermore, where CDR sequences have not been characterised, it is a matter of routine for the skilled person to determine one or more of the CDRs once provided with the full-length light and/or heavy variable chains, using routine methods (such as computational algorithms or mutation/binding assays).

A thirty-seventh aspect of the present invention provides a method of producing a composition that comprises multiple copies of a binding molecule having binding specificity and/or binding affinity to an epitope within ECD1 (and optionally ECD3) of a US28 protein of human cytomegalovirus (HCMV), wherein said binding molecule comprises the, or each, of the structure(s) (e.g. CDR sequences, or the) that have been identified within a selected candidate binding molecule as providing its binding characteristics, in accordance with the method of the thirty-sixth aspect of the present invention, said method comprising causing the reproduction of the binding molecule.

The thirty-seventh aspect of the present invention further provides a composition of binding molecule obtained by the same aspect, for example pharmaceutically acceptable composition. Said binding molecules or compositions thereof may then be used in any way discussed herein in the context of the binding molecules for the first aspect of the present invention.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The present application also provides aspects according to the following numbered paragraphs:

1. A binding molecule, comprising one or more polypeptide chains, said binding molecule having binding specificity to a first epitope within a polypeptide consisting of the amino acid sequence of TDVLNQSKPVTL (SEQ ID NO: 177) within the extracellular domain 1 (ECD1) of a US28 protein of human cytomegalovirus (HCMV), and
optionally, wherein the binding molecule also has binding specificity to a second epitope within extracellular domain 3 (ECD3) of a US28 protein of HCMV (and/or wherein said binding molecule is a first binding molecule that is formulated with a second binding molecule that has binding specificity to a second epitope within ECD3 of a US28 protein of HCMV),
wherein ECD1, SEQ ID NO: 177, and ECD3 of the US28 protein each comprise an amino acid sequence presented in the US28 protein at positions corresponding to positions 1 to 37, 26 to 37, and 167 to 183, respectively, of the US28 protein encoded by human cytomegalovirus (HCMV) as set forth in SEQ ID NO: 5.

2. The binding molecule of paragraph 1, wherein:
(a) the binding molecule is selected from an antibody and a chimeric antigen receptor (CAR); and/or
(b) the binding molecule is a multispecific (for example, bispecific or trispecific) binding molecule that comprises one or more regions with binding specificity to the first epitope within a polypeptide consisting of the amino acid sequence of SEQ ID NO: 177 within ECD1 and further comprises one or more regions with binding specificity to the second epitope within ECD3, respectively of the US28 protein encoded by HCMV.

3. The binding molecule of paragraph 1 or 2, wherein the first epitope is present entirely within the amino acid sequence of SEQ ID NO: 177 (and/or optionally wherein the second epitope is present entirely within ECD3) of the US28 protein of HCMV.

4. The binding molecule of any preceding paragraph, wherein the first epitope is a linear epitope within the amino acid sequence of SEQ ID NO: 177 (and/or optionally wherein the second epitope is a linear epitope within ECD3) of the US28 protein of HCMV.

5. The binding molecule of any preceding paragraph, having binding specificity to the first epitope within the amino acid sequence of SEQ ID NO: 177 (and/or optionally to the second epitope within ECD3) of a US28 protein of HCMV that is HCMV strain agnostic,
for example, wherein the binding molecule: (a) has a binding specificity to the first and/or second epitope(s) within a US28 protein of HCMV that is agnostic to two or more (such as all) of HCMV strains, (b) has a binding specificity that is agnostic to ECD3 4D-variant strains and ECD3 4N-variant strains, and/or (c) has a binding specificity that is agnostic to two or more (such as all) HCMV strains selected from the group consisting of DB, Towne, AF1, VHL/E, AD169, BL, DAVIS, JP, Merlin, PH, TB40/E, Toledo, TR and VR1814 (FIX).

6. The binding molecule of any preceding paragraph, wherein the binding molecule has binding specificity to the first epitope within a polypeptide consisting of the amino acid sequence of SEQ ID NO: 177 within the ECD1, and also has binding specificity to the second epitope within ECD3, of the US28 protein of HCMV, irrespective of whether the ECD3 of the US28 protein comprises the sequence of a 4D-variant or a 4N-variant:
wherein the 4D-variant comprises the sequence of TKKDNQCMTDYDYLEVS (SEQ ID NO:7) as found in ECD3 of US28 as encoded by a first group of HCMV strains, such as Towne, VR1814, TB40/E, Merlin, JP, Ad169, AF1, VHL/E, BL and DAVIS; and
wherein the 4N-variant comprises the sequence of TKKNNQCMTDYDYLEVS (SEQ ID NO: 6) as found in ECD3 of US28 as encoded by a second group of HCMV strains, such as Toledo, TR and DB strains.

7. The binding molecule of any preceding paragraph comprising one, two, three, four, five or six complementarity determining regions (CDRs) having binding specificity to the first epitope within a polypeptide consisting of the amino acid sequence of SEQ ID NO: 177 corresponding to any one, two, three, four, five or all six of the CDR sequences of antibody:
(a) 4H3C3 as defined by SEQ ID Nos: 196, 197, 198, 199, 200 and 201, respectively, and/or a functional variant of any one or more of said CDR sequences of antibody 4H3C3;
(b) 7B1F3 as defined by SEQ ID Nos: 219, 220, 221, 199, 222 and 223, respectively, and/or a functional variant of any one or more of said CDR sequences of antibody 7B1F3; and/or
(c) 2F5B11 as defined by SEQ ID Nos: 242, 243, 244, 199, 245 and 246, respectively, and/or a functional variant of any one or more of said CDR sequences of antibody 2F5B11;
and optionally further comprises one, two, three, four, five or six complementarity determining regions (CDRs) having binding specificity to the second epitope within ECD3 of a US28 protein of HCMV, corresponding to any one, two, three, four, five or all six of the CDR sequences of antibody:
(d) 1D3 as defined by SEQ ID Nos: 8, 9, 10, 14, 15 and 16, respectively, and/or a functional variant of any one or more of said CDR sequences of antibody 1D3;
(e) 1C10 as defined by SEQ ID Nos: 112, 113, 114, 117, 83 and 118, respectively, and/or a functional variant of any one or more of said CDR sequences of antibody 1C10;
(f) 1A10 as defined by SEQ ID Nos: 112, 113, 114, 117, 83 and 118, respectively, and/or a functional variant of any one or more of said CDR sequences of antibody 1A10;
(g) 1G9 as defined by SEQ ID Nos: 76, 77, 78, 82, 83 and 84, respectively, and/or a functional variant of any one or more of said CDR sequences of antibody 1G9; and/or
(h) IE8 as defined by SEQ ID Nos: 76, 95, 96, 82, 99 and 100, respectively, and/or a functional variant of any one or more of said CDR sequences of antibody 1E8;
and
optionally wherein the binding molecule is selected from an antibody and a CAR.

8. The binding molecule of paragraph 7, comprising:
(a) one, two, or all three, of the CDR 1, 2, and 3, sequences of the variable heavy chain (V_{H}) of an antibody having binding specificity to the first epitope within a polypeptide consisting of the amino acid sequence of SEQ ID NO: 177, selected from:
   (i) 4H3C3, as defined by SEQ ID Nos: 196, 197, and 198, respectively;
   (ii) 7B1F3, as defined by SEQ ID Nos: 219, 220, and 221, respectively; and/or
   (iii) 2F5B11, as defined by SEQ ID Nos: 242, 243, and 244, respectively;
   and optionally further comprising one, two, or all three, of the CDR 1, 2, and 3, sequences of the variable heavy chain (VA) of an antibody having binding specificity to the second epitope within ECD3 of a US28 protein of HCMV, selected from:
   (iv) 1D3, as defined by SEQ ID Nos: 8, 9, and 10, respectively;
   (v) 1C10, as defined by SEQ ID Nos: 112, 113, and 114, respectively;
   (vi) 1A10, as defined by SEQ ID Nos: 112, 113, and 114, respectively;
   (vii) 1G9, as defined by SEQ ID Nos: 76, 77, and 78, respectively; and/or
   (viii) 1E8, as defined by SEQ ID Nos: 76, 95, and 96, respectively; and/or
(b) one, two, or all three, of the CDR 1, 2, and 3, sequences of the variable light chain (V_{L}) of an antibody having binding specificity to the first epitope within a polypeptide consisting of the amino acid sequence of SEQ ID NO: 177, selected from:
   (i) 4H3C3, as defined by SEQ ID Nos: 199, 200, and 201, respectively;
   (ii) 7B1F3, as defined by SEQ ID Nos: 199, 222, and 223, respectively; and/or
   (iii) 2F5B11, as defined by SEQ ID Nos: 199, 245, and 246, respectively;
   and optionally further comprising one, two, or all three, of the CDR 1, 2, and 3, sequences of the variable light chain (V_{L}) of an antibody having binding specificity to the second epitope within ECD3 of a US28 protein of HCMV, selected from:
   (iv) 1D3, as defined by SEQ ID Nos: 14, 15, and 16, respectively;
   (v) 1C10, as defined by SEQ ID Nos: 117, 83, and 118, respectively;
   (vi) 1A10, as defined by SEQ ID Nos: 117, 83, and 118, respectively;
   (vii) 1G9, as defined by SEQ ID Nos: 82, 83, and 84, respectively; and/or
   (viii) 1E8, as defined by SEQ ID Nos: 82, 99, and 100, respectively.

9. The binding molecule of paragraph 7 or 8, comprising:
(a) at least one variable heavy chain (V_{H}) polypeptide that comprises CDR 1, 2, and 3 sequences of an antibody having binding specificity to the first epitope within a polypeptide consisting of the amino acid sequence of SEQ ID NO: 177, having the sequences of:
   (i) SEQ ID Nos: 196, 197, and 198 of antibody 4H3C3, respectively, and
   optionally wherein the at least one variable heavy chain (V_{H}) polypeptide comprises, consists essentially of, or consists of, the sequence of SEQ ID NO: 187;
   (ii) SEQ ID Nos: 219, 220, and 221 of antibody 7B1F3, respectively, and
   optionally wherein the at least one variable heavy chain (V_{H}) polypeptide comprises, consists essentially of, or consists of, the sequence of SEQ ID NO: 211;
   (iii) SEQ ID Nos: 242, 243, and 244 of antibody 2F5B11, respectively, and
   optionally wherein the at least one variable heavy chain (V_{H}) polypeptide comprises, consists essentially of, or consists of, the sequence of SEQ ID NO: 233; and/or
      (36) at least one variable light chain (V_{L}) polypeptide that comprises CDR 1, 2 and 3 sequences of an antibody having binding specificity to the first epitope within a polypeptide consisting of the amino acid sequence of SEQ ID NO: 177, having the sequences:
   (iv) SEQ ID Nos: 199, 200, and 201 of antibody 4H3C3, respectively, and
   optionally wherein the at least one variable heavy chain (V_{H}) polypeptide comprises, consists essentially of, or consists of, the sequence of SEQ ID NO: 189;
   (v) SEQ ID Nos: 199, 222, and 223 of antibody 7B1F3, respectively, and
   optionally wherein the at least one variable heavy chain (V_{H}) polypeptide comprises, consists essentially of, or consists of, the sequence of SEQ ID NO: 213;
   (vi) SEQ ID Nos: 199, 245, and 246 of antibody 2F5B11, respectively, and
   optionally wherein the at least one variable heavy chain (V_{H}) polypeptide comprises, consists essentially of, or consists of, the sequence of SEQ ID NO: 235; and
   optionally
   wherein the binding molecule further comprises at least one variable heavy chain (V_{H}) polypeptide that comprises CDR 1, 2, and 3 sequences of an antibody having binding specificity to the second epitope within ECD3 of a US28 protein of HCMV, having the sequences of:
   (vii) SEQ ID Nos: 8, 9, and 10, respectively, and
   optionally wherein the at least one variable heavy chain (V_{H}) polypeptide comprises, consists essentially of, or consists of, the sequence of SEQ ID NO: 1.2;
   (viii) SEQ ID Nos: 112, 113, and 114, respectively, and
   optionally wherein the at least one variable heavy chain (V_{H}) polypeptide comprises, consists essentially of, or consists of, the sequence of SEQ ID NO: 104;
   (ix) SEQ ID Nos: 112, 113, and 114, respectively, and
   optionally wherein the at least one variable heavy chain (V_{H}) polypeptide comprises, consists essentially of, or consists of, the sequence of SEQ ID NO: 122;
   (x) SEQ ID Nos: 76, 77, and 78, respectively, and
   optionally wherein the at least one variable heavy chain (V) polypeptide comprises, consists essentially of, or consists of, the sequence of SEQ ID NO: 68; and/or
   (xi) SEQ ID Nos: 76, 95, and 96, respectively, and
   optionally wherein the at least one variable heavy chain (V_{H}) polypeptide comprises, consists essentially of, or consists of, the sequence of SEQ ID NO: 88;
and/or
wherein the binding molecule further comprises at least one variable light chain (V_{L}) polypeptide that comprises CDR 1, 2 and 3 sequences of an antibody having binding specificity to the second epitope within ECD3 of a US28 protein of HCMV, having the sequences of:
   (xii) SEQ ID Nos: 14, 15, and 16, respectively, and
   optionally wherein the variable light chain (V_{L}) polypeptide comprises, consists essentially of, or consists of, the sequence of SEQ ID NO: 18;
   (xiii) SEQ ID Nos: 117, 83, and 118, respectively, and
   optionally wherein the variable light chain (V_{L}) polypeptide comprises, consists essentially of, or consists of, the sequence of SEQ ID NO: 108;
   (xiv) SEQ ID Nos: 117, 83, and 118, respectively, and
   optionally wherein the variable light chain (V_{L}) polypeptide comprises, consists essentially of, or consists of, the sequence of SEQ ID NO: 126;
   (xv) SEQ ID Nos: 82, 83, and 84, respectively, and
   optionally wherein the variable light chain (V_{L}) polypeptide comprises, consists essentially of, or consists of, the sequence of SEQ ID NO: 72; and/or
   (xvi) SEQ ID Nos: 82, 99, and 100, respectively, and
   optionally wherein the variable light chain (V_{L}) polypeptide comprises, consists essentially of, or consists of, the sequence of SEQ ID NO: 92.

10. The binding molecule of any preceding paragraph, wherein the binding molecule is selected from the group consisting of:
(a) bivalent antibodies, such as IgG-scFv antibodies (for example, wherein a first binding domain is an intact IgG and a second binding domain is an scFv attached to the first binding domain at the N-terminus of a light chain and/or at the C-terminus of a light chain and/or at the N-terminus of a heavy chain and/or at the C-terminus of a heavy chain of the IgG, or vice versa) including but not limited to bivalent antibodies with binding specificity for the first epitope within a polypeptide consisting of the amino acid sequence of TDVLNQSKPVTL (SEQ ID NO: 177) and with binding specificity for the second epitope within extracellular domain 3 (ECD3) of a US28 protein of HCMV, for example wherein the first and/or second epitope are as defined by any preceding paragraph;
(b) monovalent antibodies, such as a DuoBody® or ‘knob-in-hole’ bispecific antibody (for example, an scFv-KIH, scFv-KIH^{r}, a BiTE-KIH or a BiTE-KIH^{r};
(c) scFv₂-Fc antibodies;
(d) bispecific antibodies, such as bispecific T-cell engager (BiTE) antibodies;
(e) dual variable domain (DVD)-Ig antibodies;
(f) dual-affinity re-targeting (DART)-based antibodies (for example, DART₂-Fc or DART);
(g) trispecific antibodies, such as DNL-Fab₃ antibodies or trispecific antibodies with binding specificity for the first epitope within a polypeptide consisting of the amino acid sequence of TDVLNQSKPVTL (SEQ ID NO: 177) and with binding specificity for the second epitope within extracellular domain 3 (ECD3) of a US28 protein of HCMV, for example wherein the first and/or second epitope are as defined by any preceding paragraph, and binding specificity for a third epitope such as an epitope presented by an immune cell, optionally wherein the trispecific antibody is a trispecific immune cell engager antibody, for example, a trispecific T-cell engager (TiTE), and optionally wherein the TiTE antibody comprises a CD3-binding domain;
(h) scFv-HSA-scFv antibodies;
(i) single domain antibodies;
(j) heavy-chain-only IgGs (hcIgGs), such as camelid IgG (e.g. VHH antibodies) and shark immunoglobulin new antigen receptor (IgNAR), and single chain antibodies thereof; and
(k) a chimeric antigen receptor (CAR) comprising an extracellular domain according to paragraph 1 or any paragraph dependent thereon, for example an extracellular domain that comprises any one of options (a) to (h) of this paragraph, or combinations thereof.

11. The binding molecule of any preceding paragraph, wherein the binding molecule is a:
(i) a bispecific immune cell engager antibody, for example, a bispecific T-cell engager (BiTE), and optionally wherein the BiTE antibody comprises a CD3-binding domain; or
(ii) monoclonal antibody, optionally a recombinant monoclonal antibody, for example, a monoclonal antibody produced recombinantly by CHO cells.

12. A functional fragment of a binding molecule as defined by any of the preceding paragraphs, wherein the functional fragment:
(a) comprises or consists of an antigen-binding fragment of a binding molecule as defined by any of the preceding paragraphs, or a variant, fusion or derivative thereof selected from the group consisting of: an Fv fragment (such as a single chain Fv fragment (scFv), or a disulphide-bonded Fv fragment), a Fab-like fragment (such as a Fab fragment, a Fab′ fragment or a F(ab)₂ fragment), and single domain antibodies (dAbs, including single and dual formats, such as dAb-linker-dAb and nanobodies);
(b) provides the binding characteristics as defined by any of paragraphs 1-6;
(c) comprises the CDR sequences as defined by any one of paragraphs 7-9; and/or
(d) comprises the V_{H} and/or V_{L} sequences as defined by paragraph 9.

13. A binding molecule as defined by any of Paragraphs 1-11, or a functional fragment of said binding molecule as defined by Paragraph 12, wherein the binding molecule or the functional fragment thereof comprises a fusion polypeptide sequence,
said fusion polypeptide sequence comprising a first amino acid sequence fused a second amino acid sequence, wherein
the first amino acid sequence comprises or consists of at least one of the polypeptide chains of the binding molecule or of the functional fragment thereof, and
the second amino acid sequence is a fusion partner.

14. A chimeric antigen receptor (CAR) comprising:
(i) an extracellular domain, wherein the extracellular domain comprises or consists of a binding molecule as defined by any of Paragraphs 1-11 or 13, or a functional fragment of said binding molecule as defined by Paragraph 12 or 13;
(ii) a transmembrane domain; and
(iii) an intracellular domain;
wherein the extracellular domain of the CAR has binding specificity to a first epitope within a polypeptide consisting of the amino acid sequence of TDVLNQSKPVTL (SEQ ID NO: 177) within ECD1 (and optionally also has binding specificity to a second epitope within ECD3) of a US28 protein of human cytomegalovirus (HCMV), and
wherein ECD1, SEQ ID NO: 177, and ECD3 of the US28 protein each comprise an amino acid sequence presented in the US28 protein at positions corresponding to positions 1 to 37, 26 to 37, and 167 to 183, respectively, of the US28 protein encoded by human cytomegalovirus (HCMV) as set forth in SEQ ID NO: 5.

15. A CAR according to Paragraph 14, wherein:
(a) the extracellular domain of the CAR has binding specificity to a first epitope present entirely within a polypeptide consisting of the amino acid sequence of TDVLNQSKPVTL (SEQ ID NO: 177) within ECD1 (and/or has binding specificity to a second epitope present entirely within ECD3) of the US28 protein of HCMV;
(b) the extracellular domain of the CAR has binding specificity to a first linear epitope within a polypeptide consisting of the amino acid sequence of TDVLNQSKPVTL (SEQ ID NO: 177) within ECD1 (and/or has binding specificity to a second linear epitope within ECD3) of the US28 protein;
(c) the extracellular domain of the CAR has binding specificity to a first epitope within a polypeptide consisting of the amino acid sequence of TDVLNQSKPVTL (SEQ ID NO: 177) within ECD1, and/or a second epitope within ECD3 of a US28 protein of HCMV that is HCMV strain agnostic, for example, binding specificity to an epitope within SEQ ID NO: 177 within ECD1 and/or ECD3 of a US28 protein of HCMV that is agnostic to two or more (such as all) of HCMV strains selected from the group consisting of DB, Towne, AD169, BL, DAVIS, JP, Merlin, PH, TB40/E, Toledo, TR, VHL/E and VR1814 (FIX); and/or
(d) the extracellular domain of the CAR has specificity to an epitope within ECD3 of the US28 protein of HCMV, irrespective of whether the ECD3 of the US28 protein comprises the sequence of:
TKKDNQCMTDYDYLEVS (SEQ ID NO:7) as found in ECD3 of US28 as encoded by a majority of HCMV strains, or
TKKNNQCMTDYDYLEVS (SEQ ID NO: 6) as found in ECD3 of US28 as encoded by a minority of HCMV strains.

16. A CAR according to Paragraph 14 or 15, wherein:
(a) the extracellular domain of the CAR comprises one, two, three, four, five or six complementarity determining regions (CDRs) having binding specificity to the first epitope within a polypeptide consisting of the amino acid sequence of SEQ ID NO: 177 corresponding to any one, two, three, four, five or all six of the CDR sequences of antibody:
   (i) 41-13C3 as defined by SEQ ID Nos: 196, 197, 198, 199, 200 and 201, respectively, and/or a functional variant of any one or more of said CDR sequences of antibody 4H3C3;
   (ii) 7B1F3 as defined by SEQ ID Nos: 219, 220, 221, 199, 222 and 223, respectively, and/or a functional variant of any one or more of said CDR sequences of antibody 7B1F3; and/or
   (iii) 2F5B11 as defined by SEQ ID Nos: 242, 243, 244, 199, 245 and 246, respectively, and/or a functional variant of any one or more of said CDR sequences of antibody 2F5B11;
   and optionally further comprises one, two, three, four, five or six complementarity determining regions (CDRs) having binding specificity to the second epitope within ECD3 of a US28 protein of HCMV, corresponding to any one, two, three, four, five or all six of the CDR sequences of antibody:
   (iv) 1D3 as defined by SEQ ID Nos: 8, 9, 10, 14, 15 and 16, respectively, and/or a functional variant of any one or more of said CDR sequences of antibody 1D3;
   (v) 1C10 as defined by SEQ ID Nos: 112, 113, 114, 117, 83 and 118, respectively, and/or a functional variant of any one or more of said CDR sequences of antibody 1C10;
   (vi) 1A10 as defined by SEQ ID Nos: 112, 113, 114, 117, 83 and 118, respectively, and/or a functional variant of any one or more of said CDR sequences of antibody 1A10;
   (vii) 1G9 as defined by SEQ ID Nos: 76, 77, 78, 82, 83 and 84, respectively, and/or a functional variant of any one or more of said CDR sequences of antibody 1G9; and/or
   (viii) 1E8 as defined by SEQ ID Nos: 76, 95, 96, 82, 99 and 100, respectively, and/or a functional variant of any one or more of said CDR sequences of antibody 1E8;
      (37) the extracellular domain of the CAR comprises:
   one, two, or all three, of the CDR 1, 2, and 3, sequences of the variable heavy chain (V_{H}) of an antibody having binding specificity to the first epitope within a polypeptide consisting of the amino acid sequence of SEQ ID NO: 177, selected from:
      (38) 4H3C3 as defined by SEQ ID Nos: 196, 197, and 198, respectively;
   (ii) 7B1F3 as defined by SEQ ID Nos: 219, 220, and 221, respectively; and/or
   (iii) 2F5B11 as defined by SEQ ID Nos: 242, 243, and 244, respectively;
and optionally further comprises one, two, or all three, of the CDR 1, 2, and 3, sequences of the variable heavy chain (V_{H}) of an antibody having binding specificity to the second epitope within ECD3 of a US28 protein of HCMV, selected from:
   (iv) 1D3, as defined by SEQ ID Nos: 8, 9, and 10, respectively;
   (v) 1C10, as defined by SEQ ID Nos: 112, 113, and 114, respectively;
   (vi) 1A10, as defined by SEQ ID Nos: 112, 113, and 114, respectively;
   (vii) 1G9, as defined by SEQ ID Nos: 76, 77, and 78, respectively; and/or
   (viii) 1E8, as defined by SEQ ID Nos: 76, 95, and 96, respectively; and/or
one, two, or all three, of the CDR 1, 2, and 3, sequences of the variable light chain (V_{L}) of an antibody having binding specificity to the first epitope within a polypeptide consisting of the amino acid sequence of SEQ ID NO: 177, selected from:
   (ix) 4H3C3 as defined by SEQ ID Nos: 199, 200, and 201, respectively;
   (x) 7B1F3 as defined by SEQ ID Nos: 199, 222, and 223, respectively; and/or
(xi) 2F5B11 as defined by SEQ ID Nos: 199, 245, and 246, respectively;
and optionally further comprises one, two, or all three, of the CDR 1, 2, and 3, sequences of the variable light chain (V_{L}) of an antibody having binding specificity to the second epitope within ECD3 of a US28 protein of HCMV, selected from:
   (xii) 1D3, as defined by SEQ ID Nos: 14, 15, and 16, respectively;
   (xiii) 1C10, as defined by SEQ ID Nos: 117, 83, and 118, respectively;
   (xiv) 1A10, as defined by SEQ ID Nos: 117, 83, and 118, respectively;
   (xv) 1G9, as defined by SEQ ID Nos: 82, 83, and 84, respectively; and/or
   (xvi) 1E8, as defined by SEQ ID Nos: 82, 99, and 100, respectively; and/or
      the extracellular domain of the CAR comprises:
at least one variable heavy chain (V_{H}) polypeptide sequence that comprises CDR 1, 2, and 3 sequences of an antibody having binding specificity to the first epitope within a polypeptide consisting of the amino acid sequence of SEQ ID NO: 177, selected from:
   (i) SEQ ID Nos: 196, 197, and 198 of antibody 4H3C3, respectively, and optionally wherein the at least one variable heavy chain (V_{H}) polypeptide sequence comprises, consists essentially of, or consists of, the sequence of SEQ ID NO: 187;
   (ii) SEQ ID Nos: 219, 220, and 221 of antibody 7B1F3, respectively, and optionally wherein the at least one variable heavy chain (V_{H}) polypeptide sequence comprises, consists essentially of, or consists of, the sequence of SEQ ID NO: 211;
   (iii) SEQ ID Nos: 242, 243, and 244 of antibody 2F5B11, respectively, and optionally wherein the at least one variable heavy chain (V_{H}) polypeptide sequence comprises, consists essentially of, or consists of, the sequence of SEQ ID NO: 233; and/or
at least one variable light chain (V_{L}) polypeptide sequence that comprises CDR 1, 2 and 3 sequences of an antibody having binding specificity to the first epitope within a polypeptide consisting of the amino acid sequence of SEQ ID NO: 177, selected from:
   (iv) SEQ ID Nos: 199, 200, and 201 of antibody 4H3C3, respectively, and optionally wherein the at least one variable heavy chain (V_{L}) polypeptide sequence comprises, consists essentially of, or consists of, the sequence of SEQ ID NO: 189;
   (v) SEQ ID Nos: 199, 222, and 223 of antibody 7B1F3, respectively, and optionally wherein the at least one variable heavy chain (V_{L}) polypeptide sequence comprises, consists essentially of, or consists of, the sequence of SEQ ID NO: 213;
   (vi) SEQ ID Nos: 199, 245, and 246 of antibody 2F5B11, respectively, and optionally wherein the at least one variable heavy chain (V_{L}) polypeptide sequence comprises, consists essentially of, or consists of, the sequence of SEQ ID NO: 235; and
   optionally wherein the extracellular domain of the CAR further comprises at least one further variable heavy chain (V_{H}) polypeptide sequence that comprises CDR 1, 2, and 3 sequences of an antibody having binding specificity to the second epitope within ECD3 of a US28 protein of HCMV, selected from:
   (vii) SEQ ID Nos: 8, 9, and 10, respectively, and optionally wherein the at least one variable heavy chain (V_{H}) polypeptide sequence comprises, consists essentially of, or consists of, the sequence of SEQ ID NO: 12;
   (viii) SEQ ID Nos: 112, 113, and 114, respectively, and
   optionally wherein the at least one variable heavy chain (V_{H}) polypeptide comprises, consists essentially of, or consists of, the sequence of SEQ ID NO: 104;
   (ix) SEQ ID Nos: 112, 113, and 114, respectively, and
   optionally wherein the at least one variable heavy chain (V_{H}) polypeptide comprises, consists essentially of, or consists of, the sequence of SEQ ID NO: 122;
   (x) SEQ ID Nos: 76, 77, and 78, respectively, and
   optionally wherein the at least one variable heavy chain (V) polypeptide comprises, consists essentially of, or consists of, the sequence of SEQ ID NO: 68; and/or
   (xi) SEQ ID Nos: 76, 95, and 96, respectively, and
   optionally wherein the at least one variable heavy chain (V_{H}) polypeptide comprises, consists essentially of, or consists of, the sequence of SEQ ID NO: 88; and/or
wherein the extracellular domain of the CAR further comprises at least one further variable light chain (V_{L}) polypeptide sequence that comprises CDR 1, 2 and 3 sequences of an antibody having binding specificity to the second epitope within ECD3 of a US28 protein of HCMV, selected from:
   (xii) SEQ ID Nos: 14, 15, and 16, respectively, and optionally wherein the variable light chain (V_{L}) polypeptide comprises, consists essentially of, or consists of, the sequence of SEQ ID NO: 1.8;
   (xiii) SEQ ID Nos: 117, 83, and 118, respectively, and
   optionally wherein the variable light chain (V_{L}) polypeptide comprises, consists essentially of, or consists of, the sequence of SEQ ID NO: 108;
   (xiv) SEQ ID Nos: 117, 83, and 118, respectively, and
   optionally wherein the variable light chain (V_{L}) polypeptide comprises, consists essentially of, or consists of, the sequence of SEQ ID NO: 126;
   (xv) SEQ ID Nos: 82, 83, and 84, respectively, and
   optionally wherein the variable light chain (V_{L}) polypeptide comprises, consists essentially of, or consists of, the sequence of SEQ ID NO: 72; and/or
   (xvi) SEQ ID Nos: 82, 99, and 100, respectively, and
   optionally wherein the variable light chain (V_{L}) polypeptide comprises, consists essentially of, or consists of, the sequence of SEQ ID NO: 92.

17. A CAR according to any of Paragraphs 14-16, wherein the extracellular domain is an antibody, for example a single-chain variable fragment (scFv).

18. A CAR according to any of Paragraphs 14-17, wherein the transmembrane domain comprises the transmembrane domain of a protein, for example the transmembrane domain of a transmembrane receptor protein, and
optionally wherein the transmembrane domain comprises the transmembrane domain of a protein selected from the group consisting of the alpha, beta or zeta chain of the T-cell receptor, CD28, CD3 epsilon, CD8, CD45 and CD4.

19. A CAR according to any of Paragraphs 14-18, wherein the extracellular domain is connected to the transmembrane domain by a hinge region.

20. A CAR according to any of Paragraphs 14-19, wherein the intracellular domain comprises an intracellular signalling domain, for example wherein:
(a) the intracellular signalling domain comprises one or more immunoreceptor tyrosine-based activation motifs (ITAMs); and/or
(b) the intracellular signalling domain comprises a signalling domain of CD3 zeta, Fc receptor gamma, Fc receptor beta, CD3 gamma, CD3 delta, CD3 epsilon, CD5, CD22, CD79a, CD79b, and CD66d.

21. A CAR according to Paragraph 20, wherein the intracellular domain comprises one or more costimulatory domains, for example:
(a) wherein the one or more costimulatory domains includes one or more functional signalling domains obtained from a protein selected from the group consisting of CD28, 41BB, OX40, ICOS, CD27, and DAP10;
(b) wherein the intracellular domain incorporates a costimulatory domain proximal to the intracellular signalling domain,
wherein the intracellular domain comprises two or more costimulatory domains, for example two in-line costimulatory domains, and/or
(d) wherein the intracellular domain incorporates separate cytokine signals.

22. A CAR according to any of Paragraphs 14-21, wherein the CAR further comprises a leader sequence.

23. A nucleic acid molecule, or combination of multiple distinct nucleic acid molecules,
wherein the nucleic acid molecule comprises, or the combination of multiple distinct nucleic acid molecules collectively comprises, one or more nucleic acid sequences that, individually or in combination, encode the binding molecule of any of Paragraphs 1-13, or the CAR of any of Paragraphs 14-22.

24. A vector comprising a nucleic acid molecule, or combination of multiple distinct nucleic acid molecules, according to Paragraph 23.

25. A vector according to Paragraph 24 wherein the vector is selected from the group consisting of a retroviral vector, a plasmid, a lentivirus vector, and an adenoviral vector.

26. A cell comprising the nucleic acid molecule, or combination of multiple distinct nucleic acid molecules, according to Paragraph 23, or the vector according to Paragraphs 24 or 25,
optionally wherein the cell expresses one or more binding molecules according to any of Paragraphs 1-13, and/or one or more CARs according to of any of Paragraphs 14-22, said one or more binding molecules and/or CARs being encoded by the nucleic acid molecule, or combination of multiple distinct nucleic acid molecules, according to Paragraph 23, or the vector according to Paragraphs 24 or 25.

27. A cell according to Paragraph 26, wherein the cell comprises:
(a) a nucleic acid molecule, or combination of multiple distinct nucleic acid molecules, according to Paragraph 23, wherein the encoded binding molecule is an antibody according to any of Paragraphs 1-11, a functional fragment of said antibody according to Paragraph 12, or an antibody of functional fragment thereof that comprises a fusion polypeptide sequence according to Paragraph 13; and/or
(b) a vector according to Paragraph 25 or 26, wherein said vector comprises a nucleic acid molecule, or combination of multiple distinct nucleic acid molecules as defined by part (a) of this paragraph.

28. A cell according to Paragraph 26, wherein the cell comprises:
(a) a nucleic acid molecule, or combination of multiple distinct nucleic acid molecules, according to Paragraph 23, wherein the encoded binding molecule is a CAR according to any of Paragraphs 14-22; and/or
(b) a vector according to Paragraph 25 or 26, wherein said vector comprises a nucleic acid molecule, or combination of multiple distinct nucleic acid molecules as defined by part (a) of this paragraph.

29. A cell comprising a binding molecule according to any of Paragraphs 1-13 and/or a nucleic acid encoding said binding molecule, optionally wherein said nucleic acid is a nucleic acid or vector as defined by any of Paragraphs 23 to 25.

30. The cell of Paragraph 29, wherein
the binding molecule is an antibody according to any of Paragraphs 1-11, a functional fragment of said antibody according to Paragraph 12, or an antibody of functional fragment thereof that comprises a fusion polypeptide sequence according to Paragraph 13, and
optionally wherein the antibody is monoclonal antibody, and further for example wherein the cell is a CHO cell that recombinantly expresses the monoclonal antibody

31. A cell comprising a CAR according to any of Paragraphs 14-22 and/or a nucleic acid encoding said CAR, optionally wherein said nucleic acid is a nucleic acid or vector as defined by any of Paragraphs 23 to 25.

32. A cell according to Paragraph 26, 28, 29 or 31, or a method according to Paragraph 32, wherein the cell is a T cell, natural killer (NK) cell, or a macrophage.

33. A cell according to Paragraph 31 or 32, wherein the cell is a CAR-T cell, a CAR-NK cell or a CAR-macrophage, and
optionally, when the cell is a CAR-T cell, then the T-cell is selected from the group consisting of CD8⁺ T cells, CD4⁺ T cells, effector T cells, helper T cells, memory T cells, cytotoxic T lymphocytes (CTLs), EBV-specific T cell receptor (TCR) or γδ-T cell subtypes.

34. A method of producing a cell, the method comprising introducing a nucleic acid molecule, or combination of multiple distinct nucleic acid molecules, according to Paragraph 23, and/or a vector according to Paragraphs 24 or 25, into a cell.

35. A method of producing a binding molecule according to any of Paragraphs 1 to 13, or a CAR according to any of Paragraphs 14 to 22, the method comprising:
expressing a nucleic acid molecule, or combination of multiple distinct nucleic acid molecules, according to Paragraph 23, and/or a vector according to Paragraphs 24 or 25, in a cell.

36. The method of Paragraph 35, comprising the step of isolating the thus-produced binding molecule from the cell; and
optionally, wherein the binding molecule is an antibody according to any of Paragraphs 1-11, a functional fragment of said antibody according to Paragraph 12, or an antibody of functional fragment thereof that comprises a fusion polypeptide sequence according to Paragraph 13.

37. An isolated binding molecule that is obtained, or obtainable, by the method of Paragraph 36,
optionally, wherein the isolated binding molecule is further formulated for administration to a subject.

38. A conjugate, the conjugate comprising a moiety conjugated to a binding molecule as defined by any of Paragraphs 1-11 or 13, or to a functional fragment of said binding molecule as defined by Paragraph 12 or 13.

39. The conjugate of Paragraph 38, wherein said moiety is a therapeutic, prophylactic, diagnostic, prognostic, or theragnostic moiety.

40. The conjugate of Paragraph 38 or 39, wherein said moiety is a drug (for example, wherein the conjugate is an antibody-drug conjugate (“ADC”)) and/or a radioactive moiety (for example, wherein the conjugate is suitable for use in radioimmunotherapy (“RIT”)).

41. A method of producing a conjugate according to any of Paragraphs 38 to 40, the method comprising the steps of:
(a) providing a binding molecule as defined by any of Paragraphs 1-11 or 13, or a functional fragment of said binding molecule as defined by Paragraph 12 or 13; and
(b) conjugating a moiety to the binding molecule as defined by any of Paragraphs 1-11 or 13, or to the functional fragment of said binding molecule as defined by Paragraph 12 or 13.

42. The method of Paragraph 41, comprising the step of isolating the thus-produced conjugate.

43. The conjugate of any of Paragraphs 38 to 40, or the method of Paragraph 41 or 42, wherein the binding molecule is an antibody according to any of Paragraphs 1-11, a functional fragment of said antibody according to Paragraph 12, or an antibody of functional fragment thereof that comprises a fusion polypeptide sequence according to Paragraph 13.

44. An isolated conjugate that is obtained, or obtainable, by the method of Paragraph 42 or 43,
optionally, wherein the isolated conjugate is further formulated for administration to a subject.

45. A method of combating HCMV or a disease or condition associated with HCMV, the method comprising administering to a subject, or to ex vivo or in vitro cellular material, any one or more agents selected from the group consisting of:
i. a binding molecule according to any of Paragraphs 1-11 or 13,
ii. a functional fragment of said binding molecule as defined by Paragraph 12 or 13,
iii. an isolated binding molecule according to Paragraph 37,
iv. a nucleic acid molecule, or combination of multiple distinct nucleic acid molecules, according to Paragraph 23,
v. a vector according to Paragraphs 24 or 25,
vi. a cell according to any of Paragraphs 26 to 33,
vii. a conjugate according to any of Paragraphs 38 to 40, and
viii. an isolated conjugate according to Paragraph 44.

46. One or more agents for use in combating a disease or condition associated with HCMV in a subject, or in ex vivo or in vitro cellular material, wherein the one or more agents is, or are, each individually selected from the group consisting of:
i. a binding molecule according to any of Paragraphs 1-11 or 13,
ii. a functional fragment of said binding molecule as defined by Paragraph 12 or 13,
iii. an isolated binding molecule according to Paragraph 37,
iv. a nucleic acid molecule, or combination of multiple distinct nucleic acid molecules, according to Paragraph 23,
v. a vector according to Paragraphs 24 or 25,
vi. a cell according to any of Paragraphs 26 to 33,
vii. a conjugate according to any of Paragraphs 38 to 40, and
viii. an isolated conjugate according to Paragraph 44.

47. Use one or more agents in the manufacture of a medicament for combating a disease or condition associated with HCMV in a subject, or in ex vivo or in vitro cellular material, wherein the one or more agents is, or are, each individually selected from the group consisting of:
i. a binding molecule according to any of Paragraphs 1-11 or 13,
ii. a functional fragment of said binding molecule as defined by Paragraph 12 or 13,
iii. an isolated binding molecule according to Paragraph 37,
iv. a nucleic acid molecule, or combination of multiple distinct nucleic acid molecules, according to Paragraph 23,
v. a vector according to Paragraphs 24 or 25,
vi. a cell according to any of Paragraphs 26 to 33,
vii. a conjugate according to any of Paragraphs 38 to 40, and
viii. an isolated conjugate according to Paragraph 44.

48. A method according to Paragraph 45, the one or more agents for use according to Paragraph 46, or the use according to Paragraph of 47, wherein:
i. the disease or condition is an HCMV infection or is associated with an HCMV infection, and optionally the HCMV infection comprises a multi-strain HCMV infection;
ii. the disease or condition is a latent HCMV infection (optionally a multi-strain latent HCMV infection) or is associated with a latent HCMV infection (optionally a multi-strain latent HCMV infection);
iii. the disease or condition is a lytic HCMV infection (optionally a multi-strain lytic HCMV infection) or is associated with a lytic HCMV infection (optionally a multi-strain lytic HCMV infection);
iv. the disease or condition is a congenital single or multi-strain HCMV infection, such as a latent congenital single or multi-strain HCMV infection or a lytic congenital single or multi-strain HCMV infection;
v. the disease or condition is cancer;
vi, the disease or condition is a HCMV-infected cancer (optionally a multi-strain HCMV infected cancer), such as latent HCMV-infected cancer (optionally a multi-strain latent HCMV infected cancer);
vii. the disease or condition is an epithelial cancer; optionally wherein the epithelial cancer is breast cancer; for example, wherein the breast cancer is triple negative breast cancer (TNBC), or a HER2-positive breast cancer (such as a triple positive breast cancer “TPBC”);
viii. the disease is a metastasising and/or aggressive form of cancer, such as a metastasising and/or aggressive form of HCMV-infected cancer (e.g. a cancer having a latent HCMV infection and/or a multi-strain HCMV infection);
ix. the disease or condition is not glioblastoma;
x. the subject in need thereof has been diagnosed with HCMV-infected cancer cells, such as latent HCMV-infected cancer cells; and/or
xi. the subject in need thereof is, or is intended to be, the recipient of an organ donation, or an organ donor; and
wherein the multi-strain HCMV infection as described herein comprises infection with more than one different strains of HCMV, for example:
a) multiple HCMV strains that show inter-strain sequence variation in one or more positions in the region of positions 1 to 25 of ECD1 (such as any one or more of positions 8, 15, 18, 19, 24, 25, including but not limited to the sequence variations listed in Table 3 of the present application), and/or
b) one or more HCMV strain that encodes the 4N-variant of ECD3 of US28 and one or more HCMV strain that encodes the 4D-variant of ECD3 of US28.

49. A method according to Paragraph 45 or 48, the one or more agents for use according to Paragraph 46 or 48, or the use according to Paragraph of 47 or 48,
wherein the agent is selected from the group consisting of:
i. a therapeutic antibody as defined by any of Paragraphs 1 to 11,
ii. a functional fragment of said therapeutic antibody as defined by Paragraph 12,
iii. a therapeutic antibody that comprises as fusion polypeptide sequence as defined by Paragraph 13; and
iv. a functional fragment of said therapeutic antibody that comprises as fusion polypeptide sequence as defined by Paragraph 13.

50. A method according to Paragraph 45 or 48, the one or more agents for use according to Paragraph 46 or 48, or the use according to Paragraph of 47 or 48,
wherein the agent is a bispecific antibody, for example a bispecific immune cell engager antibody, such as bispecific T-cell engager (BiTE) antibody, optionally wherein the BiTE antibody comprises a CD3-binding domain.

51. A method according to Paragraph 45 or 48, the one or more agents for use according to Paragraph 46 or 48, or the use according to Paragraph of 47 or 48,
wherein the agent is a conjugate according to any of Paragraphs 38 to 40, such as conjugate that is an antibody-drug conjugate (“ADC”), or a conjugate that comprises radioactive moiety, such as conjugate that is suitable for use in radioimmunotherapy (“RIT”).

52. A method according to Paragraph 45 or 48, the one or more agents for use according to Paragraph 46 or 48, or the use according to Paragraph of 47 or 48,
wherein the agent is a cell comprising a CAR according to any of Paragraphs 31 to 33, for example a CAR-T cell, a CAR-NK cell or a CAR-macrophage.

53. A method according to Paragraph 45, or any of Paragraphs 48 to 52, the one or more agents for use according to Paragraph 46 or any of Paragraphs 48 to 52, or the use according to any of Paragraphs 47 to 52,
wherein the subject is one who is administered a further substance, such as a further therapeutic, prophylactic, diagnostic, prognostic, or theragnostic substance, and
optionally wherein the further substance is administered separately, sequentially or simultaneously with the, or each of the one or more agents.

54. An agent for use in medicine, wherein the agent is selected from the group consisting:
i. a binding molecule according to any of Paragraphs 1-11 or 13,
ii. a functional fragment of said binding molecule as defined by Paragraph 12 or 13,
iii. an isolated binding molecule according to Paragraph 37,
iv. a nucleic acid molecule, or combination of multiple distinct nucleic acid molecules, according to Paragraph 23,
V. a vector according to Paragraphs 24 or 25,
vi. a cell according to any of Paragraphs 26 to 33,
vii. a conjugate according to any of Paragraphs 38 to 40, and
viii. an isolated conjugate according to Paragraph 44.

55. A vaccine composition suitable for use in vaccinating against, reducing the risk of, preventing, or combating a disease or condition associated with human cytomegalovirus (HCMV),
wherein the vaccine is an active or passive vaccine,
wherein the vaccine triggers and/or provides an immune response directed to a first epitope present within a polypeptide consisting of the amino acid sequence of TDVLNQSKPVTL (SEQ ID NO: 177) within ECD1 of a US28 protein of HCMV (and, optionally, wherein the vaccine also triggers and/or provides an immune response directed to a second epitope present within ECD3 of a US28 protein of HCMV), and
wherein ECD1, SEQ ID NO: 177, and ECD3 of the US28 protein comprises an amino acid sequence presented in the US28 protein at positions corresponding to positions 1 to 37, 26 to 37, and 167 to 183, respectively, of the US28 protein encoded by HCMV as set forth in SEQ ID NO:5.

56. The vaccine composition of Paragraph 55, wherein the vaccine triggers and/or provides an immune response:
(a) to one or more epitopes present entirely within a polypeptide consisting of the amino acid sequence of TDVLNQSKPVTL (SEQ ID NO: 177) within ECD1 of the US28 protein of HCMV and/or to one or more epitopes present entirely within ECD3 of the US28 protein of HCMV;
(b) to one or more linear epitopes within a polypeptide consisting of the amino acid sequence of TDVLNQSKPVTL (SEQ ID NO: 177) within ECD1 of the US28 protein of HCMV and/or to one or more linear epitopes within ECD3 of the US28 protein of HCMV;
(c) to one or more epitopes within ECD3 of a US28 protein of HCMV that is an epitope, or are epitopes, present in identical form in both the 4D-variant strains and 4N-variant strains of HCMV, wherein the 4D-variant strain of HCMV encodes a US28 protein comprising an ECD3 having the sequence of TKKDNQCMTDYDYLEVS (SEQ ID NO:7) and wherein the 4N-variant strain of HCMV encodes a US28 protein comprising an ECD3 having the sequence of TKKNNQCMTDYDYLEVS (SEQ ID NO:6);
(d) wherein the immune response that is triggered or provided by the vaccine is HCMV strain agnostic to multiple HCMV strains that show inter-strain sequence variation in one or more positions in the region of positions 1 to 25 of ECD1 (such as any one or more of positions 8, 15, 18, 19, 24, 25, including but not limited to the sequence variations listed in Table 3 of the present application), for example wherein the multiple strains are two, three, four, five, six, seven, eight, nine or ten different strains selected from the group consisting of DB, Toledo, Towne, VR1814, TB40/E, Merlin, AD169, VHL/E, Davis and BL; and/or
(e) wherein the immune response that is triggered or provided by the vaccine is HCMV strain agnostic to the 4D-variant strains and 4N-variant strains of HCMV, and triggers and/or provides an immune response that is directed to one or more of the 4D-variant HCMV strains selected from Towne, VR1814, TB40/E, Merlin, JP, Ad169, VHL/E, AF1, BL and DAVIS and is also directed to one or more of the 4N-variant HCMV strains selected from Toledo, TR and DB.

57. The vaccine composition of Paragraph 55 or 56, wherein:
(i) the vaccine is a passive vaccine, and/or optionally comprises:
   one or more binding molecules according to any of Paragraphs 1-11 or 13,
   one or more functional fragments of said one or more binding molecules as defined by Paragraph 12 or 13,
   one or more isolated binding molecules according to Paragraph 37,
   one or more nucleic acid molecules, or combination of multiple distinct nucleic acid molecules, according to Paragraph 23,
   one or more vectors according to Paragraphs 24 or 25,
   one or more cells according to any of Paragraphs 26 to 33,
   one or more conjugates according to any of Paragraphs 38 to 40, and/or
   one or more isolated conjugates according to Paragraph 44; and/or
(ii) the vaccine is an active vaccine, and/or optionally comprises:
   (a) one or more peptides or polypeptides according to any of Paragraphs 84 to 88, a combination of at least two distinct peptides and/or polypeptides according to Paragraph 89, a fusion protein according to Paragraph 90, a combination of at least two distinct fusion proteins according to Paragraph 91, a conjugate according to any of Paragraphs 92 to 94, and/or a combination of at least two distinct conjugates according to Paragraph 95; and/or
   (b) one or more nucleic acid molecules, or combination of multiple distinct nucleic acid molecules, according to Paragraph 101 or 102, or the vector according to Paragraphs 103 or 104; and/or
   (c) a dendritic cell, or a homogeneous or heterogeneous population thereof, wherein the or each dendritic cells is loaded with one or more of the following: a peptide or polypeptide according to any of Paragraphs 84 to 88, a combination of at least two distinct peptides and/or polypeptides according to Paragraph 89, a fusion protein according to Paragraph 90, a combination of at least two distinct fusion proteins according to Paragraph 91, a conjugate according to any of Paragraphs 92 to 94, a combination of at least two distinct conjugates according to Paragraph 95, a nucleic acid molecule, or combination of multiple distinct nucleic acid molecules, according to Paragraph 101 or 102, and/or one or more vector according to Paragraphs 103 or 104.

58. A method of vaccinating against, reducing the risk of, preventing, and/or combating a disease or condition associated with HCMV, the method comprising administering to a subject a vaccine according to any of Paragraphs 55 to 57,
optionally wherein the disease or condition is a latent HCMV infection, or is a disease or condition associated with a latent HCMV infection.

59. A vaccine according to any of Paragraphs 55 to 57 for use in vaccinating against, reducing the risk of, preventing, and/or combating a disease or condition associated with HCMV in a subject,
optionally wherein the disease or condition is a latent HCMV infection, or is a disease or condition associated with a latent HCMV infection.

60. Use of a vaccine according to any of Paragraphs 55 to 57 in the manufacture of a medicament for vaccinating against, reducing the risk of, preventing, and/or combating a disease or condition associated with HCMV in a subject,
optionally wherein the disease or condition is a latent HCMV infection, or is a disease or condition associated with a latent HCMV infection.

61. The vaccine composition of any of Paragraphs 55 to 57, the method of Paragraph 58, the vaccine composition for use according to Paragraph 59, or the use according to Paragraph of 60, wherein:
i. the disease or condition is an HCMV infection or is associated with an HCMV infection, and optionally the HCMV infection comprises a multi-strain HCMV infection;
ii. the disease or condition is a latent HCMV infection (optionally a multi-strain latent HCMV infection) or is associated with a latent HCMV infection (optionally a multi-strain latent HCMV infection);
iii. the disease or condition is a lytic HCMV infection (optionally a multi-strain lytic HCMV infection) or is associated with a lytic HCMV infection (optionally a multi-strain lytic HCMV infection);
iv. the disease or condition is a congenital single or multi-strain HCMV infection, such as a latent congenital single or multi-strain HCMV infection or a lytic congenital single or multi-strain HCMV infection;
v. the disease or condition is cancer;
vi. the disease or condition is a HCMV-infected cancer (optionally a multi-strain HCMV infected cancer), such as latent HCMV-infected cancer (optionally a multi-strain latent HCMV infected cancer);
vii. the disease or condition is an epithelial cancer; optionally wherein the epithelial cancer is breast cancer; for example, wherein the breast cancer is triple negative breast cancer (TNBC), or a HER2-positive breast cancer (such as a triple positive breast cancer “TPBC”);
viii. the disease or condition is a metastasising and/or aggressive form of cancer, such as a metastasising and/or aggressive form of HCMV-infected cancer (e.g. a cancer having a latent HCMV infection and/or a multi-strain HCMV infection);
ix. the disease or condition is not glioblastoma;
x. the subject in need thereof has been diagnosed with HCMV-infected cancer cells, such as latent HCMV-infected cancer cells; and/or
xi. the subject in need thereof is, or is intended to be, the recipient of an organ donation, or an organ donor;
wherein the multi-strain HCMV infection as described herein comprises infection with more than one different strains of HCMV, for example:
a) multiple HCMV strains that show inter-strain sequence variation in one or more positions in the region of positions 1 to 25 of ECD1 (such as any one or more of positions 8, 15, 18, 19, 24, 25, including but not limited to the sequence variations listed in Table 3 of the present application), and/or
b) one or more HCMV strain that encodes the 4N-variant of ECD3 of US28 and one or more HCMV strain that encodes the 4D-variant of ECD3 of US28.

62. A method of assessing one or more biological conditions and/or biological characteristics of a subject and/or of ex vivo biological material, wherein the method comprises:
(a) contacting the subject and/or the ex vivo biological material with a binding molecule as defined by any of Paragraphs 1 to 13, or a conjugate as defined by any of Paragraphs 38 to 40, 43 or 44; and
(b) making an assessment of the subject and/or the ex vivo biological material based on a direct and/or indirect measurement of the binding of the binding molecule or conjugate to the subject and/or the ex vivo biological material.

63. The method of Paragraph 62, wherein the method comprises an ELISA method, and optionally the method is performed on ex vivo biological material, such as one or more body fluids.

64. The method of Paragraph 62, wherein the method comprises a flow cytometry method, and optionally the method is a method for assessing an ex vivo blood sample and/or ex vivo bone marrow sample from a subject.

65. The method of Paragraph 62, wherein the method comprises the use of a conjugate as defined by any of Paragraphs 38 to 40, 43 or 44,
wherein the conjugate comprises a detectable moiety, such as radioactive moiety, and the method comprises detection of the radioactive moiety in the subject and/or the ex vivo biological material,
for example, wherein the method is a method of immune-positron emitting (PET) imaging.

66. The method of Paragraph 62, wherein the method is a method of immunohistochemistry, performed on a sample of ex vivo biological material.

67. The method of any of Paragraphs 62 to 66, wherein the method is performed on a subject, or on ex vivo biological material obtained from the subject, for the purposes of making a diagnostic or prognostic assessment of a disease or condition associated with HCMV in the subject.

68. The method of Paragraph 67, wherein:
i. the disease or condition is an HCMV infection or is associated with an HCMV infection, and optionally the HCMV infection comprises a multi-strain HCMV infection;
ii. the disease or condition is a latent HCMV infection (optionally a multi-strain latent HCMV infection) or is associated with a latent HCMV infection (optionally a multi-strain latent HCMV infection);
iii. the disease or condition is a lytic HCMV infection (optionally a multi-strain lytic HCMV infection) or is associated with a lytic HCMV infection (optionally a multi-strain lytic HCMV infection);
iv. the disease or condition is a congenital single or multi-strain HCMV infection, such as a latent congenital single or multi-strain HCMV infection or a lytic congenital single or multi-strain HCMV infection;
v. the disease or condition is cancer;
vi. the disease or condition is a HCMV-infected cancer (optionally a multi-strain HCMV infected cancer), such as latent HCMV-infected cancer (optionally a multi-strain latent HCMV infected cancer);
vii. the disease or condition is an epithelial cancer; optionally wherein the epithelial cancer is breast cancer; for example, wherein the breast cancer is triple negative breast cancer (TNBC), or a HER2-positive breast cancer (such as a triple positive breast cancer “TPBC”);
viii. the disease or condition is a metastasising and/or aggressive form of cancer, such as a metastasising and/or aggressive form of HCMV-infected cancer (e.g. a cancer having a latent HCMV infection and/or a multi-strain HCMV infection);
ix. the disease or condition is not glioblastoma; and/or
x. the subject in need thereof has been diagnosed with HCMV-infected cancer cells, such as latent HCMV-infected cancer cells or cancer cells with a productive HCMV infection (including but not limited to tumour associated macrophages with productive HCMV infection), and/or multi-strain HCMV infected cancer cells;
xi. the subject is, or is intended to be, the recipient of an organ donation, or an organ donor;
wherein the multi-strain HCMV infection as described herein comprises infection with more than one different strains of HCMV, for example:
a) multiple HCMV strains that show inter-strain sequence variation in one or more positions in the region of positions 1 to 25 of ECD1 (such as any one or more of positions 8, 15, 18, 19, 24, 25, including but not limited to the sequence variations listed in Table 3 of the present application), and/or
b) one or more HCMV strain that encodes the 4N-variant of ECD3 of US28 and one or more HCMV strain that encodes the 4D-variant of ECD3 of US28.

69. A method of combating a HCMV infection (such as a latent HCMV infection and/or a lytic HCMV infection and/or a multi-strain HCMV infection) in living ex vivo biological material, the method comprising contacting the living ex vivo biological material with any one or more agents selected from the group consisting of:
i. a binding molecule according to any of Paragraphs 1-11 or 13,
ii. a functional fragment of said binding molecule as defined by Paragraph 12 or 13,
iii. an isolated binding molecule according to Paragraph 37,
iv. a nucleic acid molecule, or combination of multiple distinct nucleic acid molecules, according to Paragraph 23,
v. a vector according to Paragraphs 24 or 25,
vi. a cell according to any of Paragraphs 26 to 33,
vii. a conjugate according to any of Paragraphs 38 to 40, and
viii. an isolated conjugate according to Paragraph 44.

70. Living ex vivo biological material that is obtained, or obtainable, by the method of Paragraph 69.

71. The method of Paragraph 69, or living ex vivo biological material according to Paragraph 70, wherein the ex vivo living biological material comprises, consists essentially of, or consists of, living ex vivo biological material selected from the group that includes: one or more types of ex vivo cells; one or more types of ex vivo cell cultures; one or more types of ex vivo tissues; one or more types of ex vivo tissue cultures; one or more types of ex vivo organoids; one or more types of ex vivo organoid cultures; one or more types of ex vivo organs; and/or one or more types of ex vivo organ cultures.

72. A method of treating a subject in need thereof, comprising administering ex vivo living biological material as defined by Paragraph 60 or 61, to the subject.

73. The method of Paragraph 72, wherein the method is a method of transplantation of the ex vivo living biological material, such as an organ or tissue transplant.

74. A method of screening for a binding molecule having binding specificity to a first epitope within a polypeptide consisting of the amino acid sequence of TDVLNQSKPVTL (SEQ ID NO: 177) within ECD1 of a US28 protein of human cytomegalovirus (HCMV),
wherein ECD1 and SEQ ID NO: 177 of the US28 protein comprises an amino acid sequence presented in the US28 protein at positions corresponding to positions 1 to 37 and 26 to 27, respectively, of the US28 protein encoded by HCMV as set forth in SEQ ID NO:5,
the method comprising:
(a) providing one or more peptides or polypeptides comprising, consisting essentially of, or consisting of, the sequence TDVLNQSKPVTL (SEQ ID NO: 177), or an immunogenic fragment thereof,
optionally wherein the one or more peptides or polypeptides are provided in the form of:
   one or more peptides or polypeptides according to any of Paragraphs 84 to 88,
   a combination of at least two distinct peptides and/or polypeptides according to Paragraph 89,
   one or more fusion proteins according to Paragraph 90,
   a combination of at least two distinct fusion proteins according to Paragraph 91, a conjugate according to any of Paragraphs 92 to 94,
   or a combination of at least two distinct conjugates according to Paragraph 95, and
preferably wherein the only US28-derived sequence present within said one or more peptides or polypeptides is the sequence of SEQ ID NO: 177 of an immunogenic fragment thereof, such as a fragment that is bound by antibody 4H3C3, 7B1F3 and/or 2F5B11;
(b) providing one or more candidate binding molecules;
(c) determining the binding specificity and/or binding affinity of one or more candidate binding molecules to the one or more peptides, thereby to select one or more binding molecules having binding specificity to the first epitope within a polypeptide consisting of the amino acid sequence of SEQ ID NO: 177; and
optionally, wherein the method further involves screening said binding molecules for binding specificity to an epitope within extracellular domain 3 (ECD3) of a US28 protein of human cytomegalovirus (HCMV), by additional method steps (which may be performed either before, or after steps (a)-(c)), said additional method steps comprising:
providing one or more peptides or polypeptides corresponding an amino acid sequence present in ECD3 of the US28 protein,
optionally wherein the one or more peptides or polypeptides are provided in the form of:
   one or more peptides or polypeptides according to any of Paragraphs 85 to 88,
   a combination of at least two distinct peptides and/or polypeptides according to options (b) and (c) of Paragraph 89,
   one or more fusion proteins according to Paragraph 90 comprising the sequence of at least one peptide or polypeptide having the sequence of SEQ ID NO: 6 and/or 7 or an immunogenic fragment of either, according to any of Paragraphs 85 to 88,
   a combination of at least two distinct fusion proteins according to options (b) and (c) of Paragraph 91,
   a conjugate according to any of Paragraphs 92 to 94 comprising the sequence of at least one peptide or polypeptide having the sequence of SEQ ID NO: 6 and/or 7 or an immunogenic fragment of either, according to any of Paragraphs 85 to 88, or
   a combination of at least two distinct conjugates according to options (b) and (c) of Paragraph 95;
preferably wherein the only US28-derived sequence present within said one or more peptides is the sequence of SEQ ID NO: 6 and/or 7, or an immunogenic fragment of either, such as a fragment that is bound by antibody 1D3, 1C10, 1A10, 1G9, and/or 1E8;
(ii) providing one or more candidate binding molecules, optionally wherein steps (a)-(c) are performed before steps (i)-(iii) and wherein said candidate binding molecules for step (ii) are a product of step (c) of this paragraph; and
(iii) determining the binding specificity and/or binding affinity of one or more candidate binding molecules to the one or more peptides or polypeptides corresponding an amino acid sequence present in ECD3 of the US28 protein, optionally wherein steps (a)-(c) are performed after steps (i)-(iii) and wherein said candidate binding molecules for step (b) are a product of step (iii) of this paragraph.

75. The method of Paragraph 74, wherein the, or each, of the one or more peptides comprise, consist essentially of, or consist of, the polypeptide sequence TDVLNQSKPVTL (SEQ ID NO: 177), TKKDNQCMTDYDYLEVS (SEQ ID NO:7) and/or TKKNNQCMTDYDYLEVS (SEQ ID NO: 6), and/or an immunogenic fragment of either or each;
optionally wherein the one or more peptides are provided in the form of one or more peptides or polypeptides according to any of Paragraphs 84 to 88, a combination of at least two distinct peptides and/or polypeptides according to Paragraph 89, one or more fusion proteins according to Paragraph 90, a combination of at least two distinct fusion proteins according to Paragraph 91, a conjugate according to any of Paragraphs 92 to 94, or a combination of at least two distinct conjugates according to Paragraph 95.

76. The method of Paragraph 74 or 75, wherein the one or more candidate binding molecules are antibodies and/or CARs, such as any one or more of:
(a) bivalent antibodies, such as IgG-scFv antibodies (for example, wherein a first binding domain is an intact IgG and a second binding domain is an scFv attached to the first binding domain at the N-terminus of a light chain and/or at the C-terminus of a light chain and/or at the N-terminus of a heavy chain and/or at the C-terminus of a heavy chain of the IgG, or vice versa) including but not limited to bivalent antibodies with binding specificity for the first epitope within a polypeptide consisting of the amino acid sequence of TDVLNQSKPVTL (SEQ ID NO: 177) and with binding specificity the second epitope within extracellular domain 3 (ECD3) of a US28 protein of HCMV, for example wherein the first and/or second epitope are as defined by any of Paragraphs 1 to 9;
(b) monovalent antibodies, such as a DuoBody® or ‘knob-in-hole’ bispecific antibody (for example, an scFv-KIH, scFv-KIH^{r}, a BiTE-KIH or a BiTE-iKIH^{r};
(c) scFv₂-Fc antibodies;
(d) bispecific antibodies, such as bispecific T-cell engager (BiTE) antibodies;
(e) dual variable domain (DVD)-Ig antibodies;
(f) dual-affinity re-targeting (DART)-based antibodies (for example, DART₂-Fc or DART);
(g) trispecific antibodies, such as DNL-Fab₃ antibodies or trispecific antibodies with binding specificity for the first epitope within a polypeptide consisting of the amino acid sequence of TDVLNQSKPVTL (SEQ ID NO: 177) and with binding specificity the second epitope within extracellular domain 3 (ECD3) of a US28 protein of HCMV, for example wherein the first and/or second epitope are as defined by any of Paragraph 1 to 9, and binding specificity for a third epitope such as an epitope presented by an immune cell, optionally wherein the trispecific antibody is a trispecific immune cell engager antibody, for example, a trispecific T-cell engager (TiTE), and optionally wherein the TiTE antibody comprises a CD3-binding domain;
(h) scFv-HSA-scFv antibodies; and
(i) a chimeric antigen receptor (CAR).

77. The method of any of Paragraphs 74 to 76, further comprising the step of selecting a candidate binding molecule based on the binding specificity and/or binding affinity to the one or more peptides.

78. The method of Paragraph 77, wherein the selected candidate binding molecule:
(a) has binding specificity to a first epitope present entirely within a polypeptide consisting of the amino acid sequence of SEQ ID NO: 177 within ECD1 (and optionally a second epitope present entirely within ECD3) of the US28 protein of HCMV;
(b) has binding specificity to a first linear epitope within a polypeptide consisting of the amino acid sequence of SEQ ID NO: 177 within ECD1 (and optionally has binding specificity to a second linear epitope within ECD3) of the US28 protein;
(c) has binding specificity to a first epitope within a polypeptide consisting of the amino acid sequence of SEQ ID NO: 177 within ECD1 and/or a second epitope within ECD3 of a US28 protein of HCMV that is HCMV strain agnostic, for example, binding specificity to an epitope within a polypeptide consisting of the amino acid sequence of SEQ ID NO: 177 within ECD1 and/or ECD3 of a US28 protein of HCMV that is agnostic to two or more (such as all) of HCMV strains selected from the group consisting of DB, Towne, AD169, DAVIS, BL, JP, Merlin, PH, TB40/E, Toledo, VHL/E, TR and VR1814 (FIX);
(d) has specificity to an epitope within ECD3 of the US28 protein of HCMV, irrespective of whether the ECD3 of the US28 protein comprises the sequence of TKKDNQCMTDYDYLEVS (SEQ ID NO:7) or TKKNNQCMTDYDYLEVS (SEQ ID NO: 6); and/or
(e) has specificity to a discontinuous epitope present entirely within:
   a first region of US28 that is within a polypeptide consisting of the amino acid sequence of SEQ ID NO: 177 within ECD1; and
   a second region of US28 that is within ECD3 of US28, preferably amino acids that are identical in each of SEQ ID Nos: 6 and 7.

79. The method of Paragraph 77 or 78, wherein the selected candidate binding molecule has a binding specificity and/or binding affinity to the one or more peptides or polypeptides comprising, consisting essentially of, or consisting of, the sequence TDVLNQSKPVTL (SEQ ID NO: 177), or an immunogenic fragment thereof that is equivalent to the binding specificity and/or binding affinity to the same one or more peptides or polypeptides as demonstrated by an antibody that comprises:
(a) a variable heavy chain (V_{H}) polypeptide that consists of the sequence of SEQ ID NO:187 and a variable light chain (V_{L}) polypeptide that consists of the sequence of SEQ ID NO:189;
(b) a variable heavy chain (V_{H}) polypeptide that consists of the sequence of SEQ ID NO:211 and a variable light chain (V_{L}) polypeptide that consists of the sequence of SEQ ID NO:213; or
(c) a variable heavy chain (V_{H}) polypeptide that consists of the sequence of SEQ ID NO:233 and a variable light chain (V_{L}) polypeptide that consists of the sequence of SEQ ID NO:235.

80. A method of producing a composition that comprises multiple copies of a binding molecule, said method comprising causing the reproduction of a selected candidate binding molecule that has been selected in accordance with the method of any of Paragraphs 77, 78 or 79.

81. A method of assessing a selected candidate binding molecule that has been selected in accordance with the method of any of Paragraphs 77, 78 or 79, said method comprising and identifying the structure(s) within the selected candidate binding molecule that provides its binding characteristics, for example, identifying the, or each, CDR sequence in a selected candidate binding molecule that is an antibody or CAR.

82. A method of producing a composition that comprises multiple copies of a binding molecule, wherein said binding molecule comprises the, or each, of the structure(s) that have been identified within a selected candidate binding molecule as providing its binding characteristics, in accordance with the method of Paragraph 81,
said method comprising causing the reproduction of the binding molecule.

83. A composition of binding molecules obtained by the method of Paragraph 80 or 82.

84. A peptide or polypeptide comprising, consisting essentially of, or consisting of, the sequence TDVLNQSKPVTL (SEQ ID NO: 177), or comprising the sequence of an immunogenic fragment of SEQ ID NO: 177, wherein said peptide or polypeptide is not the US28 protein, and
preferably wherein the only US28-derived sequence in said peptide or polypeptide is the sequence of SEQ ID NO: 177 or the sequence of the immunogenic fragment of SEQ ID NO: 177.

85. A peptide or polypeptide comprising, consisting essentially of, or consisting of, the sequence TKKNNQCMTDYDYLEVS (SEQ ID NO: 6) and/or TKKDNQCMTDYDYLEVS (SEQ ID NO:7), or comprising the sequence of an immunogenic fragment of SEQ ID NO: 6 and/or 7, wherein said peptide or polypeptide is not the US28 protein, optionally wherein said peptide or polypeptide also comprises the sequence of SEQ ID NO: 177 or the sequence of the immunogenic fragment of SEQ ID NO: 177, wherein said peptide or polypeptide is not the US28 protein, and
preferably wherein the only US28-derived sequence(s) in said peptide or polypeptide is, or are, the sequence(s) of SEQ ID NO: 6 and/or 7 or the sequence(s) of the immunogenic fragment(s) of SEQ ID NO: 6 and/or 7, and optionally also the sequence of SEQ ID NO: 177 or the sequence of the immunogenic fragment of SEQ ID NO: 177.

86. The peptide or polypeptide of Paragraph 84 or 85, wherein:
(a) the immunogenic fragment of SEQ ID NO: 177 comprises less than the full sequence of SEQ ID NO: 177, and comprises at least 3, 4, 5, 6, 7, 8, 9, 10, or 11 consecutive amino acids of SEQ ID NO: 177;
(b) the immunogenic fragment of SEQ ID NO: 6 comprises less than the full sequence of SEQ ID NO: 6, and comprises at least 3, 4, 5, 6, 7, 8, 9, 10, or 11 consecutive amino acids of SEQ ID NO: 6; and/or
(c) the immunogenic fragment of SEQ ID NO: 7 comprises less than the full sequence of SEQ ID NO: 7, and comprises at least 3, 4, 5, 6, 7, 8, 9, 10, 11, 12, 13, 14, 15, or 16 consecutive amino acids of SEQ ID NO: 7.

87. The peptide or polypeptide of Paragraph 85 or 86, wherein the immunogenic fragment of SEQ ID NO: 6 and/or 7 comprises, consists essentially of, or consists of, a sequence that is common to, and present within, both of SEQ ID NO: 6 and SEQ ID NO:7.

88. A peptide or polypeptide comprising, consisting, or consisting essentially of, the sequence of an immunogenic fragment or variant of TDVLNQSKPVTL (SEQ ID NO: 177), wherein the immunogenic fragment or variant comprises the sequence of the epitope within ECD1 of US28 that is bound by antibody 4H3C3, 7B1F3 and/or 2F5B11; and
optionally further comprising, consisting, or consisting essentially of, the sequence of an immunogenic fragment or variant of TKKNNQCMTDYDYLEVS (SEQ ID NO: 6) or TKKDNQCMTDYDYLEVS (SEQ ID NO: 7), wherein the immunogenic fragment or variant comprises the sequence of the epitope within ECD3 of US28 that is bound by antibody 1D3, 1C10, 1A10, 1G9, and/or 1E8.

89. A combination of at least two distinct peptides and/or polypeptides, comprising a first peptide or polypeptide and a second peptide or polypeptide (and optionally at least a third peptide or polypeptide), wherein:
(a) the first peptide or polypeptide comprises a comprises, consists essentially of, or consists of, the sequence TDVLNQSKPVTL (SEQ ID NO: 177) according to Paragraph 84, or an immunogenic fragment thereof, such as an immunogenic fragment as defined by Paragraph 86 or 88; and
(b) the second peptide or polypeptide comprises a comprises, consists essentially of, or consists of, the sequence TKKNNQCMTDYDYLEVS (SEQ ID NO: 6) as defined by Paragraph 85, or an immunogenic fragment thereof, such as an immunogenic fragment as defined by Paragraph 86, 87 or 88, optionally with the proviso that said immunogenic fragment comprises at least the 4N amino acid of SEQ ID NO: 6; or
(c) the second peptide or polypeptide comprises a comprises, consists essentially of, or consists of, the sequence TKKDNQCMTDYDYLEVS (SEQ ID NO: 7) as defined by Paragraph 85, or an immunogenic fragment thereof, such as an immunogenic fragment as defined by Paragraph 86, 87 or 88, optionally with the proviso that said immunogenic fragment comprises at least the 4D amino acid of SEQ ID NO: 7; and
optionally wherein the combination comprises the third polypeptide and, if the second peptide or polypeptide is as defined by option (b) then the third peptide or polypeptide corresponds to option (c), or wherein if the second peptide or polypeptide is as defined by option (c) then the third peptide or polypeptide corresponds to option (b).

90. A fusion protein comprising, consisting essentially of, or consisting of, a first amino acid sequence fused, either directly or via one or more linker amino acid sequences, to a second amino acid sequence, wherein
the first amino acid sequence is the sequence of a peptide or polypeptide as defined by any of Paragraphs 84-88; and
the second amino acid sequence is a fusion partner, optionally wherein the fusion partner is a carrier protein, such as a carrier protein that is selected to provide a fusion protein that is suitable for immunisation and generation of antibodies against the first amino acid sequence,
optionally wherein the carrier protein is selected from the group consisting of keyhole limpet hemocyanin (KLH), HSA (human serum albumin), BSA (bovine serum albumin), OVA (ovalbumin), tetanus toxoid (TT), diphtheria toxoid (DT), a genetically modified cross-reacting material (CRM) of diphtheria toxin, meningococcal outer membrane protein complex (OMPC) and H. influenzae protein D (HiD).

91. A combination of at least two distinct fusion proteins, comprising a first fusion protein, and a second fusion protein (and optionally at least a third fusion protein), wherein:
(a) the first fusion protein according to Paragraph 90 comprises, as the first amino acid sequence of the first fusion protein, a sequence that comprises a comprises, consists essentially of, or consists of, the sequence TDVLNQSKPVTL (SEQ ID NO: 177) as defined by Paragraph 84, or an immunogenic fragment thereof, such as an immunogenic fragment as defined by Paragraph 86 or 88; and
(b) the second fusion protein according to Paragraph 90 comprises, as the second amino acid sequence of the first fusion protein, a sequence that comprises a comprises, consists essentially of, or consists of, the sequence TKKNNQCMTDYDYLEVS (SEQ ID NO: 6) as defined by Paragraph 85, or an immunogenic fragment thereof, such as an immunogenic fragment as defined by Paragraph 86, 87 or 88, optionally with the proviso that said immunogenic fragment comprises at least the 4N amino acid of SEQ ID NO:6; or
(c) the second fusion protein according to Paragraph 90 comprises, as the first amino acid sequence of the second fusion protein, a sequence that comprises, consists essentially of, or consists of, the sequence TKKDNQCMTDYDYLEVS (SEQ ID NO:7) as defined by Paragraph 85, or an immunogenic fragment thereof, such as an immunogenic fragment as defined by Paragraph 86, 87 or 88, optionally with the proviso that said immunogenic fragment comprises at least the 4D amino acid of SEQ ID NC):7; and
optionally wherein the combination comprises the third distinct fusion protein and, if the second fusion protein is as defined by option (b) then the third fusion protein corresponds to option (c), or wherein if the second fusion protein is as defined by option (c) then the third fusion protein corresponds to option (b).

92. A conjugate, comprising a moiety conjugated to a peptide or polypeptide as defined by any of Paragraphs 84-88, or to a fusion protein as defined by Paragraph 90.

93. The conjugate of Paragraph 92, wherein:
(i) the moiety conjugated directly to the peptide or polypeptide as defined by any of Paragraphs 84-88, or to the fusion protein as defined by Paragraph 90; or
(ii) the moiety conjugated indirectly, such as via a linker, to the peptide or polypeptide as defined by any of Paragraphs 84-88, or to the fusion protein as defined by Paragraph 90.

94. The conjugate of Paragraph 92 or 93, wherein said moiety is a carrier, for example a carrier protein, for example a carrier selected from KLH (keyhole limpet hemocyanin), HSA (human serum albumin), BSA (bovine serum albumin), OVA (ovalbumin), tetanus toxoid (TT), diphtheria toxoid (DT), a genetically modified cross-reacting material (CRM) of diphtheria toxin, meningococcal outer membrane protein complex (OMPC) and H. influenzae protein D (HiD).

95. A combination of at least two distinct conjugates, wherein the combination comprises:
(a) a first conjugate according to any of Paragraphs 92-94, wherein the first conjugate comprises, consists essentially of, or consists of, a moiety conjugated to a peptide or polypeptide, wherein the peptide or polypeptide comprises, consists essentially of, or consists of, the sequence TDVLNQSKPVTL (SEQ ID NO: 177), or an immunogenic fragment thereof, such as an immunogenic fragment as defined by Paragraph 86 or 88; and
(b) a second conjugate according to any of Paragraphs 92-94, wherein the second conjugate comprises, consists essentially of, or consists of, a moiety conjugated to a peptide or polypeptide, wherein the peptide or polypeptide comprises, consists essentially of, or consists of, the sequence TKKNNQCMTDYDYLEVS (SEQ ID NO: 6), or an immunogenic fragment thereof, such as an immunogenic fragment as defined by Paragraph 86, 87 or 88, with the proviso that said immunogenic fragment comprises at least the 4N amino acid of SEQ ID NO):6; or
(c) a second conjugate according to any of Paragraphs 92-94, wherein the second conjugate comprises, consists essentially of, or consists of, a moiety conjugated to a peptide or polypeptide, wherein the peptide or polypeptide comprises, consists essentially of, or consists of, the sequence TKKDNQCMTDYDYLEVS (SEQ ID NO:7), or an immunogenic fragment thereof, such as an immunogenic fragment as defined by Paragraph 86 or 88, with the proviso that said immunogenic fragment comprises at least the 4D amino acid of SEQ ID NO:7; and
optionally wherein the combination comprises a third distinct conjugate and, if the second conjugate is as defined by option (b) then the third conjugate corresponds to option (c), or wherein if the second conjugate is as defined by option (c) then the third conjugate corresponds to option (b).
   96. A method of producing a conjugate according to any of Paragraphs 92-94, the method comprising the steps of:
(a) providing a peptide or polypeptide as defined by any of Paragraphs 84-88, or a fusion protein as defined by Paragraph 90; and
(b) conjugating a moiety to the peptide or polypeptide as defined by any of Paragraphs 84-88, or to the fusion protein as defined by Paragraph 90.

97. A method of producing a combination of at least two distinct conjugates as defined to Paragraph 95, the method comprising the steps of:
(a) producing the first conjugate, as defined by Paragraph 90, by a method according to Paragraph 95;
(b) producing the second conjugate, as defined by Paragraph 90, by a method according to Paragraph 95;
(c) optionally producing the third conjugate, as defined by Paragraph 90, by a method according to Paragraph 95; and
(d) combining the first and second (and optionally third) conjugates, thereby to form a combination according to Paragraph 95.

98. A method of producing a combination of at least two distinct conjugates as defined to Paragraph 95, the method comprising the steps of:
(a) providing a combination of at least two distinct peptides and/or polypeptides according to Paragraph 89, or a combination of at least two distinct fusion proteins according to Paragraph 91; and
(b) conjugating a moiety to the combination of at least two distinct peptides and/or polypeptides according to Paragraph 89, or to the combination of at least two distinct fusion proteins according to Paragraph 91, thereby to form a combination according to Paragraph 95.

99. The method of Paragraph 96, 97 or 98, comprising the step of isolating the thus-produced conjugate or combination of conjugates.

100. An isolated conjugate, or combination of conjugates, that is obtained, or obtainable, by the method of any of Paragraphs 97-99,
optionally, wherein the isolated conjugate is further formulated for administration to a subject.

101. A nucleic acid molecule, or combination of multiple distinct nucleic acid molecules,
wherein the nucleic acid molecule comprises, or the combination of multiple distinct nucleic acid molecules collectively comprises, one or more nucleic acid sequences that, individually or in combination, encode one or more peptides and/or polypeptides according to any of Paragraphs 84 to 88, a combination of at least two distinct peptides and/or polypeptides according to Paragraph 89, a fusion protein according to Paragraph 90, and/or a combination of at least two distinct fusion proteins according to Paragraph 91.

102. The nucleic acid molecule, or combination of multiple distinct nucleic acid molecules, according to Paragraph 101, wherein the, or each, nucleic acid molecule is independently selected from a DNA or RNA molecule.

103. A vector comprising a nucleic acid molecule, or combination of multiple distinct nucleic acid molecules, according to Paragraph 101 or 102.

104. A vector according to Paragraph 103 wherein the vector is selected from the group consisting of a retroviral vector, a plasmid, a lentivirus vector, and an adenoviral vector.

105. A cell comprising the nucleic acid molecule, or combination of multiple distinct nucleic acid molecules, according to Paragraph 101 or 102, or the vector according to Paragraphs 103 or 104,
optionally wherein the cell expresses one or more peptide or polypeptide according to any of Paragraphs 84 to 88, a combination of at least two distinct peptides and/or polypeptides according to Paragraph 89, a fusion protein according to Paragraph 90, or a combination of at least two distinct fusion proteins according to Paragraph 91.

106. A cell that is exposed to, and/or comprising, a one or more peptide or polypeptide according to any of Paragraphs 84 to 88, a combination of at least two distinct peptides and/or polypeptides according to Paragraph 89, a fusion protein according to Paragraph 90, a combination of at least two distinct fusion proteins according to Paragraph 91, a conjugate according to any of Paragraphs 92 to 94, a combination of at least two distinct conjugates according to Paragraph 95, a nucleic acid molecule, or combination of multiple distinct nucleic acid molecules, according to Paragraph 101 or 102, or a vector according to Paragraphs 103 or 104.

107. A method of isolating and/or enriching cells comprising a T cell receptor (TCR) with specificity to a first epitope within a polypeptide consisting of the amino acid sequence of TDVLNQSKPVTL (SEQ ID NO: 177) in ECD1 (and optionally a second epitope in ECD3) of US28 (e.g. naturally occurring T cells, or recombinant cells expressing a CAR according to of any of Paragraphs 14-22, for example CAR T-cells, CAR NK-cells and/or CAR-macrophages), wherein the method comprises the step of using an agent,
wherein the agent is selected from the group consisting of a peptide or polypeptide according to any of Paragraphs 84 to 88, a combination of at least two distinct peptides and/or polypeptides according to Paragraph 89, a fusion protein according to Paragraph 90, a combination of at least two distinct fusion proteins according to Paragraph 91, a conjugate according to any of Paragraphs 92 to 94, and/or a combination of at least two distinct conjugates according to Paragraph 95,
to isolate and/or enrich cells with binding specificity to the sequence of SEQ ID Nos: 177 or an immunogenic fragment thereof, and optionally also with binding specificity to one or both of the sequences of SEQ ID Nos: 6 and/or 7, or immunogenic fragments of either.

108. The method of Paragraph 107, wherein the sequences are formulated as an MHC tetramer,
for example a Class I MHC tetramer for antigen-specific CD8+ T cells detection, a Class II MHC tetramer for antigen-specific CD4+ T cells detection, a fluorophore-labelled tetramer for flow cytometry or fluorescence microscopy.

109. The method of Paragraph 107 or 108, wherein the T-cell is selected from the group consisting of CD8⁺ T cells, CD4+ T cells, effector T cells, helper T cells, memory T cells, cytotoxic T lymphocytes (CTLs), EBV-specific T cell receptor (TCR) or γδ-T cell subtypes.

110. An MHC tetramer comprising a peptide or polypeptide according to any of Paragraphs 84 to 88, a combination of at least two distinct peptides and/or polypeptides according to Paragraph 89, optionally wherein the peptide comprises or corresponds to SEQ ID NO: 177, SEQ ID NO:6 and/or SEQ ID NO:7, or an immunogenic fragment of either or both,
for example wherein the MHC tetramer is a Class I MHC tetramer for antigen-specific CD8⁺ T cells detection, a Class II MHC tetramer for antigen-specific CD4⁺ T cells detection, a fluorophore-labelled tetramer for flow cytometry or fluorescence microscopy.

111. The MHC tetramer of Paragraph 110 for use in isolating and/or enriching cells comprising a T cell receptor (TCR) with specificity to a first epitope within a polypeptide consisting of the amino acid sequence of TDVLNQSKPVTL (SEQ ID NO: 177) in ECD1 (and optionally a second epitope in ECD3) of US28, for example, a T-cell selected from the group consisting of CD8⁺ T cells, CD4⁺ T cells, effector T cells, helper T cells, memory T cells, cytotoxic T lymphocytes (CTLs), EBV-specific T cell receptor (TCR) or γδ-T cell subtypes and/or a recombinant cell expressing a CAR according to of any of Paragraphs 14-22, for example a CAR T-cell, CAR NK-cell and/or CAR-macrophage.

The invention will now be described with the aid of the following non-limiting Examples.

### EXAMPLES

### Example 1

### Methods:

### Human Materials and Ethical Standards:

All tissue materials are collected under the highest ethical standards including approval of the protocols of the local Ethical committees and the donor being informed and signing for the informant consent. The biopsies have been collected during standard medical care and the results cannot be tracked back to the individual donors. All human tissues are collected under HIPPA approved protocols. All human samples have been tested negative for HIV and Hepatitis B and approved for commercial product development.

### Animals:

BABL/c mice were maintained in the animal facility under the guidelines of Creative Biolabs Inc. Shirley, NY 11967, USA. All animal experiments were performed in accordance with the guidelines for care and scientific use of animals of the U.S. Department of Agriculture (USDA).

### Immunization:

### ECD1

For the HCMV US28 ECD1 (i.e. N-terminal), Peptide-N was prepared via chemical synthesis: TDVLNQSKPVTL (SEQ ID NO: 177). To enhance immunogenicity, Keyhole limpet hemocyanin (KHL) was linked at the N terminal of the parent peptide. The KLH-conjugated peptide was named KLH-Peptide-N, and was used for immunization. In addition, biotinylated peptide was prepared, which was later used for antibody screening. In brief, Biotin was added to the N terminal of the parent peptide, which was renamed as Bio-Peptide-N.

Five mice (#1-5) were immunized by Peptide-N. The immunizations were repeated every 14 days. Each mouse received immunization of 60 μg of KLH-Peptide-N, in combination with complete Freund's adjuvant as the initial immunization. From the 2^{nd} boost on, each mouse received immunization of 60 μg of KLH-Peptide-N, in combination with incomplete Freund's adjuvant. After the 3^{rd} booster dose, the antiserum was collected, and the immune response was examined by Enzyme-linked immunosorbent assay (ELISA) showing positive signal against Peptide-N. The titer reached more than 64,000 in mouse 4 #.

### ECD3

For the HCMV US28 ECD3, two peptides were prepared via chemical synthesis: Peptide-1: TKKNNQCMTDYDYLEVS (SEQ ID NO: 6); Peptide-2: TKKDNQCMTDYDYLEVS (SEQ ID NO: 7). To enhance immunogenicity, Keyhole limpet hemocyanin (KHL) was linked at the N terminal of the parent peptides. The KLH-conjugated peptides were named KLH-Peptide-1 and KLH-Peptide-2, respectively, and were used for immunization. Mass spectrometry (MS) and High-performance liquid chromatography (HPLC) analysis demonstrated that the two KLH-linked peptides were highly qualified for immunization. In addition, biotinylated peptides were prepared, which were later used for antibody screening. In brief, Biotin was added to the N terminal of the parent peptides, which were renamed as Bio-Peptide-1 and Bio-Peptide-2, respectively.

Five mice (#10-15) were immunized by Peptides 1 and 2. The immunizations were repeated every 14 days. Each mouse received co-immunization of 30 μg of KLH-Peptide-1 and 30 μg of KLH-Peptide-2, in combination with complete Freund's adjuvant as the initial immunization. From the 2^{nd} boost on, each mouse received co-immunization of 30 μg of KLH-Peptide-1 and 30 μg of KLH-Peptide-2, in combination with incomplete Freund's adjuvant. After the ₃^{rd} booster dose, the antiserum was collected, and the immune response was examined by Enzyme-linked immunosorbent assay (ELISA) showing positive signal against Peptide-1 and Peptide-2, respectively. After the 4^{th} booster dose, the antiserum was collected, and the antiserum showed increased positive signal against Peptide-1 and Peptide-2, respectively. The titer reached more than 40,000 in mice 11 #, 13 #, 14 # and 15 #, and more than 10,000 in mouse 12 #.

### ECD2 and ECD4:

In contrast with the use of Peptide-N for ECD1, and Peptides-1 and -2 for ECD3, immunizations of four mice (16 #, 17 #, 18 # and 19 #) with the KHL-linked HCMV US28 ECD2 and ECD4 peptides: QYLLDHNSLAS and DTLKLLKWISSSCE, gave low if any antibody titers with the similar boosting protocol as described above. MS and HPLC analysis confirmed additionally the low immunogenicity of these peptides for immunizations. These data demonstrate that it can be challenging to identify a suitably immunogenic portion of the HCMV US28 protein, even within the sequences of the extracellular domains. The success of the ECD1- and ECD3-derived peptides, compared to the failure of the ECD2-derived and ECD4-derived peptides, was not a priori predictable.

### Hybridoma Cultures:

### ECD1

Considering antiserum ELISA results, mouse 4 # was selected for cell fusion. The spleen cells were fused with myeloma cells using Poly(ethylene glycol) (PEG) 1500 (Sigma-Aldrich, Merck KgaA, 64293 Darmstadt, Germany). Then the fused cells were seeded into 96 wells plates and cultured in semi-solid medium in the presence of hydrogen atom transfer (HAT) reagent (Cat. Number 21060017, Thermofisher, Waltham, MA 02451, USA). 958 single clones (ten 96-well plates) were tested against BSA-Peptide-N, and 137 positive clones were identified. The 2^{nd} round of screening confirmed the binding of the 107 positive clones to BSA-Peptide-N, within which 63 strong positive clones and 44 weak positive were identified. The top 30 clones were selected for subcloning, 29 of which were successfully subclones. From each parental clone, 1 subclone was selected for the 2^{nd} round ELISA test, in which all 29 subclones bound the target peptide specifically.
10 subclones were selected for further analysis, denoted as 2F5B11, 2G2B3, 4A11A11, 4H3C3, 5D6H11, 5E1E4, 5E8F7, 6G3D6, 7B1F3 and 9G3B9, and validated by FACS (see below).

### ECD3

Considering antiserum ELISA results, mice 13 # and 14 # were selected for cell fusion. The spleens of mice 13 # and 14 # were collected 5 days after the 5^{th} booster by i.v. injection of 100 μg of KLH-Peptide-1 and 100 μg of KLH-Peptide-2, respectively. The spleen cells were fused with myeloma cells using Poly(ethylene glycol) (PEG) 1500 (Sigma-Aldrich, Merck KgaA, 64293 Darmstadt, Germany). Then the fused cells were seeded into 96 wells plates and cultured in semi-solid medium in the presence of hydrogen atom transfer (HAT) reagent (Cat. Number 21060017, Thermofisher, Waltham, MA 02451, USA). QC-ELISA validation was performed against Bio-Peptide-1 and 2. 960 single clones were tested in the 1^{st} screening against Peptide-1 (coating Bio-Peptide-1), and 26 positive clones were identified. 32 clones (including the 26 positive clones above) were further studied in the 2^{nd} screening to identify clones that specifically recognize both Peptide-1 and -2. 18 positive clones were identified in the 2^{nd} screening.

Subcloning was performed for 10 clones by limiting dilution method. 5 clones each from mice 13 # and 144 were selected based on the ELISA titer in the cell fusion screening step: Mouse 13 #: 1C10, 3H9, 4C10, 4F1, and 5G6; Mouse 14 #: 1F12, 2C2, 3D11, 4F11, and 5H11. QC-ELISA validation was performed against Bio-Peptide-1. 8 positive clones were identified to bind to Peptide 1, among which 6 clones were monoclonal. In order to identify more specific clones, subcloning was performed for the remaining 8 clones from Mouse 14 #: 1D2, 1B9, 1H3, 2E3, 2G8, 2G12, 3A1 and 4E4. QC-ELISA validation was performed against Bio-Peptide-1. 7 positive clones were identified to bind to Peptide 1, among which 4 clones were monoclonal. Among these 10 monoclonal clones, additional validations showed superior binding properties for the US28-13-5G6-1D3 clone, which was selected for further studies. However, also the clones 13-1C10-1C10, 13-1C10-1G9, 14-1H3-1A10, 14-2C2-1G4 and 14-4E4-1E8 were studied further.

### Sequencing of the US28-13-5G6-1D3 Clone:

Hybridoma sequencing service by Creative Biolabs (Shirley, NY 11967, USA) was used to obtain the sequence information of the US28-13-5G6-1D3, 13-1C10-1C10, 13-1C10-1G9, 14-IH3-1A10 and 14-4E4-1E8 antibodies. Hybridoma sequencing refers to the process of obtaining sequence information regarding the cDNA encoding the variable heavy (V_{H}) and variable light (V_{L}) domains of the specific antibodies. Before sequencing, total mRNA of the selected hybridoma cells was extracted by RNA isolater (Cat. R401-01, Vazyme Biotech, Cellagen Technology LLC, San Diego, CA 92121, USA). The 5′RACE method, as described by Georgious et al. [1] was applied to amplify, clone and sequence the complete specific antibody variable regions (V_{H} and V_{L}) as well as non-variable flanking constant region sequences, which were subsequently cloned into two cloning vectors for downstream sequence manipulation and expression vector constructions. The vector constructs were then sequenced using Sanger sequencing method and used to express the recombinant antibodies (rAb) in HEK293-F cells. The antibodies were purified by Protein G chromatography and the results were checked with the SDS-PAGE on agarose gel.

### Other Antibodies:

An anti-HIS HRP antibody was ordered from GenScrip, Piscataway, NJ 08854 (Cat. Number A00612). An anti-HIS FITC antibody was ordered from Thermofisher, Waltham, MA 02451, USA (Cat. MA1-81891). A commercial polyclonal anti-US28 antibody preparation, made in rabbit, was ordered from Mybiosource, San Diego, CA 92195 (Cat. Number MBS9606669). The CMV Immediate Early Antigen Alexa488 Ab was ordered from Merck Life Sciences, Oslo, Norway (Cat. Number MAB810X) and MilliporeSigma, Burlington, MA 01803, USA (Cat. Number MAB810R). The secondary and IgG isotype control antibodies used were ordered from Thermofisher, Oslo, Norway; Goat anti-mouse IgG-Alexa 488 (Cat. Number A11001); Goat anti-rabbit IgG Alexa 488 (Cat. Number A11008); Isotype control for Goat anti-mouse IgG (Cat. Number 14-4714-82); and Goat anti-rabbit IgG (Cat. Number MA516384). The monovalent and bivalent VUN100 ScFvs were manufactured by Creative Biolabs in HEK293-F cells with recombinant technology. The VUN100 ScFvs contained a MYC tag and were detected with PE-labeled anti-Myc tag secondary antibody [9E10](ab72468, Abcam).

### Laboratory Cells:

In addition to recombinant antibody synthesis, Chinese hamster ovary (CHO-K1) cells (Creative Biolabs, Shirley, NY 11967, USA) were used to overexpress US28 protein to study the surface binding of the US28 antibodies. HEK-293 cells were also used to overexpress US28 and used for antibody binding studies. The Human Embryonic Kidney 293-F (HEK293-F) cell line was used for expression of the US28-13-5G6-1D3 recombinant antibody (rAb) (Creative Biolabs, Shirley, NY 11967, USA) and other recombinant antibodies (BioIntron Inc., Shanghai, China), although most antibodies were recombinantly manufactured using CHO cells. The Medical Research Council cell line-5 (MRC-5), which is derived from human embryo lung fibroblasts (cat #CCL-171, RRID: CVCL_0440, American Type Culture Collection (ATCC), Manassas, VA 20110 USA) was used to study antibody binding to the surface of the HCMV infected cells. Commercially available peripheral blood mononuclear cells (PBMC) were obtained from three healthy, seropositive donors (donors #509, #526, #230, Cellero, Bothell, WA 98021 USA).

### Primary Cells:

Primary cells from different tissue origin were cultured in T25 flasks coated with 3 mL Gelatin-Based Coating Solution (Cellbiologeics, 6950) and placed at 37° C. for 1h. Excess solution was aspirated before cell seeding. 15 mL centrifuge tube were prepared with 6 mL preheated complete medium. Primary cells stored in liquid nitrogen were quickly thawed in 37° C. water bath for <1 min, and transferred to the 15 mL centrifuge tube. Cells were centrifuged at 1000 rpm for 5 minutes, before aspirating the supernatant and resuspending the cell pellet in 6 mL complete medium. Resuspended cells were added into the pre-coated T25 flask, and incubated in a humidified incubator at 37° C. with 5% CO₂. Culture media was changed the following day to remove non-adherent cells and replenish nutrients, repeated daily. When cells were at >85% confluent, cell culture media was removed and discarded from the flask. The adherent layer was washed 2 times with sterile PBS (1λ). Cells were incubated with 2 mL warm (37° C.) 0.25% Trypsin-EDTA solution for 3 minutes. As soon as cells have detached, 8 mL complete medium was added to neutralize the trypsin, before being plated in fresh flasks precoated with Gelatin-Based Coating Solution in a humidified, 5% C02 incubator at 37° C. Culture media was changed the following day to remove non-adherent cells, and cells were checked daily under a microscopy to verify appropriate cell morphology. FACS analysis was performed as described herein.

### US28 Expression Constructs:

Plasmid construction was performed to create a US28-overexpressing cell line. The US28 sequence, identical to HCMV DB strain (Acc. Number: KT959235; SEQ ID No. 5 of this application), fused to a 6His tag at its C terminal part, was inserted into the pCDH-EF1a-MCS vector (Creative Biolabs, Shirley, NY 11967, USA). The plasmid construct was transfected to CHO-K1 cell line. The cells were collected 48 hours after transfection. The cell line was named as CHO-US28-A1-6His and its protein sequence (SEQ ID NO: 47) is the following:

MTPTTTTAELTTEFDYDEAATPCVFTDVLNQSKPVTLFLYGVVFL
FGSIGNFLVIFTITWRRRIQCSGDVYFINLAAADLLFVCTLPLWM
QYLLDHNSLASVPCTLLTACFYVAMFASLCFITEIALDRYYAIVY
MRYRPVKQACLFSIFWWIFAVIIAIPHFMVVTKKNNQCMTDYDYL
EVSYPIILNVELMIGAFVIPLSVISYCYYRISRIVAVSQSRHKGR
IVRVLIAVVLVFIIFWLPYHLILFVDTLKLLKWISSSCEFERSLK
RALILTESLAFCHCCLNPLLYVFVGTKFRQELHCLLAEFRQRLFS
RDVSWYHSMSFSRRSSPSRRETSSDTLSDEVCRVSQIIPAAALEG
SHHHHHH.

The plasmids carrying the DNA for the TB40/E, VHL/E, and Merlin constructs and mutated US28 strain (SEQ ID Nos: 254, 272, 273 and 255, respectively) were also manufactured in the similar manner.

SEQ ID NO: 254:
MTPTTTTTELTTEFEYDLGATPCTFTDVLNQSKPVILFLYGVVFL
FGSVGNFLVIFTITWRRRIQCSGDVYFINLAAADLLFVCTLPLWM
QYLLDANSLASVPCTLLTACFYVAMFASLCFITEIALDRYYAIVY
MRYRPVKQACLFSIFWWIFAVIIAIPHFMVVTKKDNQCMTDYDYL
EVSYPIILNVELMLGAFVIPLSVISYCYYRISRIVAVSQSRHKGR
IVRVLIAVVLVFIIFWLPYHLTLFVDTLKLLKWISSSCEFERSLK
RALILTESLAFCHCCLNPLLYVFVGTKERQELHCLLAEFRQRLES
RDVSWYHSMSFSRRSSPSRRETSSDTLSDEVCRVSQIIPAAALEG
SHHHHHH.
SEQ ID NO: 272:
MTPTTTTAELTTEFDYDDEATPCVLTDVLNQSKPVTLFLYGVVFL
FGSIGNFLVIFTITWRRRIQCSGDVYFINLAAADLLFVCTLPLWM
QYLLDANSLASVPCTLLTACFYVAMFASLCFITEIALDRYYAIVY
MRYRPVKQACLFSIFWWIFAVIIAIPHEMVVIKKDNQCMTDYDYL
EVSYPIILNVELMLGAFVIPLSVISYCYYRISRIVAVSQSRHKGR
IVRVLIAVVLVFIIFWLPYHLTLFVDTLKLLKWISSSCEFEKSLK
RALILTESLAFCHCCLNPLLYVFVGIKFRQELHCLLAEFRQRLFS
RDVSWYHSMSFSRRSSPSRRETSSDTLSDEACRVSQIIPAAALEG
SHHHHHH.
SEQ ID NO: 273:
MTPTTTTAELTTEFDYDEDATPCVFTDVLNQSKPVTLFLYGVVFL
FGSIGNFLVIFTITWRRRIQCSGDVYFINLAAADLLFVCTLPLWM
QYLLDANSLASVPCTLLTACFYVAMFASLCFITEIALDRYYAIVY
MRYRPVKQACLFSIFWWIFAVIIAIPHEMVVIKKDNQCMIDYDYL
EVSYPIILNVELMLGAFVIPLSVISYCYYRISRIVAVSQSRHKGR
IVRVLIAVVLVFIIFWLPYHLTLFVDTLKLLKWISSSCEFERSLK
RALILTESLAFCHCCLNPLLYVFVGTKFRQELHCLLAEFRQRLFS
RDVSWYHSMSFSRRGSPSRRETSSDTLSDEVCRVSQIIPAAALEG
SHHHHHH.
SEQ ID NO: 255:
MTQTTTTELTTEFDYDEGAALCTLTDVLNQSKPITLFLYGVVELF
GSIGNELVIFTITWRRRIQCSGDVYFINLAAADLLFVCTLPLWMQ
YLLDANSLASVPCTLLTACFYVAMFASICFITEIALDRYYAIVYM
RYRPVKQACLFSIFWWIFAVIIAIPHFMVVTKKDNQCMTDYDYLE
VSYPIILNVELMIGAFVIPLSVISYCYYRISRIVAVSQSRHKGRI
VRVLIAVVLVFIIFWLPYHLTLFLDTLKLLKWISSSCESEKSLKR
ALILTESLAFCHCCLNPLLYVFVGIKFRQELHCLLAEFRQRLFSR
DVSWYHSMSESRRSSPSRRETSSDTLSDEVCRVSQIIPAAALEGS
HHHHHH.

Infection of MRC-5 Cells with HCMV:

MRC-5 human embryo lung fibroblasts, passages 21 to 30, were trypsinized and 1×10⁶ cells per T75 flask were plated out in Dulbecco's modified Eagle medium (DMEM), supplemented with 10% fetal bovine serum (FBS), 2 mM L-glutamine, 0.1 mM nonessential amino acids, 10 mM HEPES, and 100 U/ml each of penicillin and streptomycin. The cells were either left uninfected (Mock) or infected with HCMV strain Ad169 (ATCC® VR538™, ATCC, VA 20110 USA) at a multiplicity of infection (m.o.i.) of 5. After adding the virus to respective cells, the cells were incubated at 37° C. with 5% CO₂ until cell harvest at day 4 post infection (p.i.) (96 hours pi). Infection was confirmed by microscopy.

### Enzyme-Linked Immunosorbent Assay (ELISA):

Microtiter plate wells were coated with 100 μl of the solution containing: (i) Bio-Peptide-N; or (ii) Bio-Peptide-1 and Bio-Peptide-2; at a concentration of between 1-10 μg/ml in coating buffer, consisting of Na₂CO₃, 1.5 g and NaHCO₃, 2.93 g in 1 L of distilled water in pH 9.6, and then incubated for 1-2 hours at room temperature. The plate was washed 3 times in wash buffer consisting of PBS containing 0.05% v/v Tween-20 and blotted with paper towels thereafter. 150 μl of blocking solution containing Phosphate buffered saline (PBS) containing 1% w/v BSA, was added to each well and the plate was incubated for 2 hours at 37° C. The wells were washed 4 times in wash buffer and blotted on paper towels after the last wash. 100 μl of antibody dilution was added to each well and the plate was incubated for 1 hour at 37° C. and thereafter washed 3 times in wash buffer. 100 μl enzyme-conjugated secondary antibody (appropriately diluted in wash buffer) was added to each well and the plate was incubated for 1 hour at 37° C. The wells were then again washed 3 times in wash buffer and blotted with paper towels after the last wash. 100 μl of the appropriate substrate solution was added to each well and incubated at room temperature for 30 minutes, or until desired color change was attained. The absorbance values were read immediately at the appropriate wavelength and the data analyzed by Cmax Plus, Molecular Devices, San Jose, CA 95134, USA,

### Western Blot:

Western blot was used to verify expression of the US28 protein in CHO-US28-A1-6His cells. The collected cells and US28 negative control CHO cells were washed with PBS twice and ice-cold RIPA lysis buffer with 1% PMSF was added. The adherent cells were scrapped gently off the dish using a plastic cell scraper and the cell suspension was transferred into a pre-cooled microcentrifuge tube, which was agitated for 30 min at 4° C. The tubes were then centrifuged, and the supernatant was aspirated into a fresh tube. The protein concentration of the tubes was determined by BCA method. 2× Laemmli buffer consisting of 4% SDS, 10% 2-mercaptoethanol, 20% glycerol, 0.004% bromophenol blue and 0.125 M Tris HCl, was added in an equal volume. The samples were reduced and denatured by boiling the lysates in sample buffer. Polyacrylamide gel (12%) was prepared and 30-120 μg protein of each sample were loaded into SDS-PAGE wells. A protein marker was loaded in the first lane. The electrophoresis was run with 1× running buffer consisting of 25 mM Tris base, 250 mM glycine, 0.1% SDS adjusted to pH 8.3 in 30 min at 80V voltage and 1.5 hours at 100 voltage. A polyvinylidene difluoride (PVDF) membrane was prepared by wetting it in methanol for 30 seconds and then soaking it briefly in distilled water followed by 1× transfer buffer, which contains 25 mM Tris base, 192 mM glycine, 20% methanol adjusted to pH 8.3. The proteins were then transferred to the PVDF membrane by a sandwich wet method in a transfer cassette. The membrane was washed in 1× Tris-Buffered Saline and Tween 20 (TBST) solution and incubated in blocking solution containing 1×TBST and 10% non-fat dry milk. The primary antibody was then diluted to working concentration in 1×TBST with 5% non-fat dry milk and incubated for 1.5 h at room temperature. The membrane was washed with 1×TBST three times and the secondary antibody was added with the same incubation and washing procedure as described above. Premixed chemiluminescent substrate solution (Thermofisher, Waltham, MA 02451, USA, Cat. 34075) was added onto the membrane, which was thereafter imaged with a chemiluminescent imaging system (BIO-RAD ChemiDocXRS+, Bio-Rad, Hercules, CA 94547, USA) for 1-90 seconds.

### The Flowcytometry (FACS) Analyses:

Flowcytometry was used to analyze cell surface binding by the US28 antibodies. Upon cell harvest, adherent non-infected and US28 overexpressing or HCMV infected cells were washed three times with cell staining buffer (phosphate-buffered saline (PBS (Amresco, Cleveland, 44139, USA) added 0.5% BSA and 0.05% Sodium Azide (NaN₃)). The cells were trypisinized and then resuspended in DMEM with 10% FBS to inhibit the action of trypsin. The cells were then pelleted at 400×g for 10 minutes and washed three times in cell staining buffer. Cell viability and cell concentration were measured by tryphan blue viability assay and a cell counter (Countess, Invitrogen, Carlsbad, CA 92008, USA). The cell suspension was adjusted to 1×10⁶ cells/mL with ice-cold cell staining buffer. The cells were blocked in 1 ml 10% FBS or 1% BSA in PBS per 1×10⁶ cells, vortexed briefly, and incubated for 10-30 minutes to eliminate non-specific staining. The cells were then stained with the respective antibodies and appropriate negative controls.

### ECD1

The primary antibodies used were I) 13-5G6-1D3 (50 μg/ml) as a positive control; II) Monovalent and Bivalent VUN100 ScFvs 1:1 (50 μg/ml); III) test antibodies for the specified subclones (1:5).

### ECD3

The primary antibodies used were I) 13-5G6-1D3 1:50 to 50 μg/ml; and II) Commercial US28 polyclonal antibody (50 μg/ml), III) Monovalent and Bivalent VUN100 ScFvs (50 μg/ml) and for certain experiments, the appropriate isotype controls were included.

In both cases (ECD1 and ECD3), the antibodies were added to the cells, vortexed and incubated on ice for 15 to 30 minutes up to overnight and washed twice with cell staining buffer to remove any unbound antibodies. In addition, the CMV Immediate Early Antigen (IE) Alexa488 Ab, MAB810X, 1:50 was included to verify HCMV infected cells in one of the experiments. Because it binds to an intracellular, HCMV encoded protein, it was added after permeabilizing the cells in 70% ethanol for 30 min on ice.

1:500-1:5000 in cell staining buffer of the appropriate fluorescent-conjugated appropriate secondary antibodies were added and incubated on ice for 15-20 min in the dark. The cells were washed three times to remove any unbound antibodies. The cells were then fixed with 0.5% paraformaldehyde (PFA) for 30 min on ice and resuspended in 200-500 μL cell staining buffer for final flow cytometric analysis using an Accuri C6 flow cytometry system (BD Biosciences, San Jose, CA 95131, USA). Data analyses were performed using FlowJo software v8.8 (Treestar Inc. Ashland, OR 97520, USA).

### Human Paraffin Embedded Tissue Arrays:

Cytomegalovirus (CMV) control slides containing CMV positive lung tissue and CMV negative myometrium (product number #3240A) were obtained from Newcomer Supply Inc., Middleton, WI 53562-2579, USA. Tissue micro-arrays (TMAs) were obtained from US Biomax Inc., Derwood, MD 20855, USA, and included tumor tissues #BR1008b, #BS17016c, #BCN721b and normal tissues #FDA662a and heart tissues #HEN241a.

### The Immunohistochemistry (IHC) Analysis:

The paraffin sections were sequentially immersed into a. 100% xylene three times (5 min each); b. 100% ethanol twice (2 min each); c. 95% ethanol twice (2 min each). The slides were rinsed twice in water for 5 min to remove ethanol. For antigen retrieval, the slides were immersed in 3% H₂O₂ for 5 minutes and washed thereafter twice (2×) with distilled water. The slides were then immersed in the working citrate buffer (pH 6.0) and covered with a lid. The buffer was heated in the microwave until it had boiled, and then kept in the steamer for 15 min.

The sections were cooled at room temperature for 30 min and washed with PBS. 5% BSA blocking solution was then added to the HIER treated samples and they were incubated at 37° C. for 30 min. The primary antibody was diluted according to the best results from the dilution gradient tests and added to the samples for incubation overnight at 4° C. or 37° C. for 1 h. The slides were then washed three times in PBS on the shaker. The secondary antibodies were used in 1:200 dilution, added to the samples and incubated at 37° C. for 30 min. The slides were washed three times (5 min each) in PBS on the shaker. Strept-Avidin Biotin Complex (SABC) HRP- or AP-conjugated reagents were added, and the samples were incubated in dark at room temperature. The samples were thereafter washed in distilled water, counter stained by hematoxylin, and then dehydrated through gradient ethanol from 70% to 100%, and cleared by 3 times in xylene, 5 min each. Slides were mounted by ACRYMOUNT (StatLab, MicKinney, TX 75069, USA) and cover-slipped. Digitized pictures were taken by Olympus VS120 scanner and analyzed by using OlyVia 3.2 software (Olympus Corporation, 20101 Tokyo, Japan) by senior Pathologist.

### In Situ Hybridization (ISH)

CMV ISH Probe (Cat No.: ISHP-031, Creative Bioarray) is designed for the detection cytomegalovirus (CMV) DNA in formalin-fixed, paraffin-embedded tissue or cells by chromogenic in situ hybridization (CISH). The CMV probe was labelled with Digoxin, using NICK methods with CMV IE1 gene vector.

The slides were first pre-treated by baking slides at 85° C. for 15 minutes, thereafter immersing slides twice in fresh xylene at RT for 5 minutes. The slides were then dehydrated in 100%, 85%, 75% ethanol at RT, 3 minutes each and immersed in PBS for 2 minutes.

The slides were thereafter pre-treated with Protease by immersing them in 0.2 M HCl for 12 minutes. 0.5% Triton X-100 was added in PBS and the slides were incubated for 12 minutes. The slides were washed twice with 1×PBS for 2 minutes, then immersed in 20 μg/ml Proteinase K for 15 minutes then again washed twice with 1×PBS for 2 minutes. Thereafter the slides were immersed in 3% H₂O₂ for 15 minutes, washed twice with 1×PBS for 2 minutes and air-dried.

To prepare the Hybridization Buffer containing probe, 1 μl of probe stock solution was added to 50 μl of Hybridization Buffer, and then vortexed and centrifuged. The ISH probe was denatured for 10 minutes at 85° C. and kept at 37° C. for 30 minutes. After slides have dried for 90+ minutes, 200 μl of hybridization buffer containing probe was dispensed onto the tissue sections of slides. A clean 24×60 mm rectangular glass coverslip was carefully placed over the hybridization solution to completely cover the tissue sections and allow for even distribution of the hybridization solution. The slides were placed in the humidified chamber, covered with the tissue culture lid, and the chamber sealed was with parafilm. The slides were incubated in the dark at 37° C. for 20-36 hours.

In situ washes were performed after removing the coverslips from sections by immersing slides for 5 minutes in 2×SSC. The slides were incubated with 3% BSA solution at 37° C. for 30 minutes and thereafter with anti-DIG-HRP solution (diluted 1:100 in 1% BSA) at 37° C. for 30 minutes. Then the slides were first washed twice with 2×SSC for 2 minutes and then twice with 1×PBS for 5 minutes. Thereafter the slides were incubated with DAB solution for 2-10 minutes and washed in running tap water. The slides were immersed in hematoxylin to counterstain for 3-5 minutes and again washed in running tap water. The slides were finally dehydrated in 100% ethanol at RT for 1 minute and air-dried and then immersed in xylene at RT for 1.0 minutes. An aqueous mounting solution was used to mount the sections.

Brightfield images were acquired using a Nikon 801 microscope equipped with a 40× objective.

### Results

### Antibody Design:

HCMV encoded US28 protein has 4 extracellular domains (ECDs), among which the 2^{nd} and 3^{rd} ECDs and parts of the ₄th ECD are highly conserved between the different HCMV strains. While the N-terminal part (ECD1) of the HCMV US28 contains frequent mutations (for example, various identified N-terminal region mutations are shown in Table 3 of the present application), the peptide used to generate binding molecules directed to ECD1 in the present application represents a potential new epitope that is particularly conserved, and so showed promise for being an appropriate immunogen for the development of antibodies against the HCMV-encoded US28 protein that are both highly specific for US28, yet are also have the potential to provide strain agnostic binding.

Repeated immunizations of mice with 5 boosts of the KHL-linked HCMV US28 ECD2 and ECD4 peptides unexpectedly resulted in low, if any, antibody titers. The low quality for immunizations of these peptides were confirmed by MS and HPLC analyses.

For the design of ECD1-derived peptides for immunization, we considered genetic variations between the different HCMV strains. A BLAST search was used, and we identified a portion of the N-terminus that was particularly well conserved across a number of HCMV clinical strains. The HCMV US28 ECD1 Peptide-N(SEQ ID NO: 177) was selected as an immunogen for the further development of strain agnostic antibodies that could recognise a spectrum of HCMV strains, thereby resulting in the 10 subclones denoted as 2F5B11, 2G2B3, 4A11A11, 4H3C3, 5D6H11, 5E1E4, 5E8F7, 6G3D6, 7B1F3 and 9G3B9, which were identified to bind to Bio-Peptide-N.

For the design of ECD3-derived peptides for immunization, we considered genetic variations between the different HCMV strains. A BLAST search was used, and we identified the existence of a single D to N variation on the 4^{th} amino acid of the US28 ECD3. 90% of the 100 randomly selected HCMV clinical strains contained the US28 ECD3 4D-variant, whereas 10% had the US28 ECD3 4N variant. Among the strains showing the US28 ECD3 4N variant were the HCMV DB (Acc. Number: KT959235), TR (Acc. Number: KF021605.1) clinical strains and Toledo (Acc. Number: GU93774) laboratory strain (Table 1), which all, based on their US28 sequence, carry the HCMV A1 strain genotype [2].

The HCMV US28 ECD3 peptide-1 and -2 (SEQ ID Nos: 6 and 7, respectively) containing both genetic variants, were selected as immunogens for the further development of strain agnostic antibodies that could recognize both 4N and 4D genotypes in immunized mice. Of the total of >1000 hybridoma clones identified from five immunized mice, 32 positive clones were obtained, of which 18 positive clones were identified to specifically recognize both Peptides-1 and -2. Subcloning of these resulted to 15 positive clones, which were identified to bind to Bio-Peptide 1, among which 10 clones were monoclonal.

### Binding Properties of the Anti-US28 Clones:

To test the obtained ten monoclonal HCMV US28 antibody clones, a US28 protein-overexpressing cell line was created by inserting a DNA sequence, encoding US28 from HCMV strain DB with C-terminal 6His tag (amino acid sequence SEQ ID NO: 47), into pCDH-EF1a-MCS vector and then transfecting the vector construct into CHO-K1 cells. The US28 overexpressing cells were thereafter called as CHO-US28-A1 cells.

The successful expression of the US28 protein in the transformed CHO-US28-A1 cells was verified through Western blot analysis by using the anti-HIS antibody binding on the 6HIS tag that was added to C-terminal part of the US28-A1 protein construct. The anti-HIS antibody marked out a specific band at ˜41 kDa, which is equivalent with the earlier reported protein size of HCMV US28, and which was not present on the control CHO-cells, thus indicating the expression of US28 protein in the transfected CHO-US28-A1 cells but not in the control CHO cells (FIG. 1).

### ECD1

FACS analysis of the obtained ten monoclonal ECD1-binding antibody clones (in 1:5 w/v Ab dilution) was performed to measure surface binding of these clones to HCMV US28 transmembrane protein on the surface of CHO-US28-A1 cells. All clones showed specific binding to CHO-US28. The negative controls without any antibody and with the mouse secondary antibody only were considered negative. 1D3-mFc (50 μg/ml) and a 1:1 ratio of monovalent and bivalent VUN100 (50 μg/ml) were used as positive controls.

### ECD3

FACS analysis of the obtained ten monoclonal antibody clones (in 1:50 w/v Ab dilution) was performed to measure surface binding of these clones to HCMV US28 transmembrane protein on the surface of CHO-US28-A1 cells. Clone US28-13-5G6-1D3 showed the best binding results, which were superior (binding—8-10-fold more to CHO-US28-A1 cells) to any of the other 9 clones (data not shown). The negative controls without any antibody and with the mice secondary antibody only were considered negative (FIG. 2).

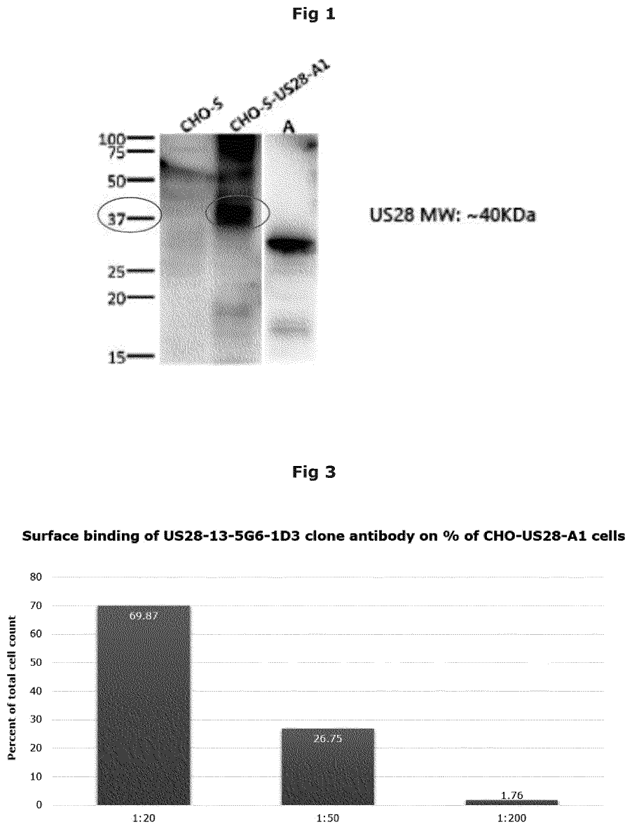

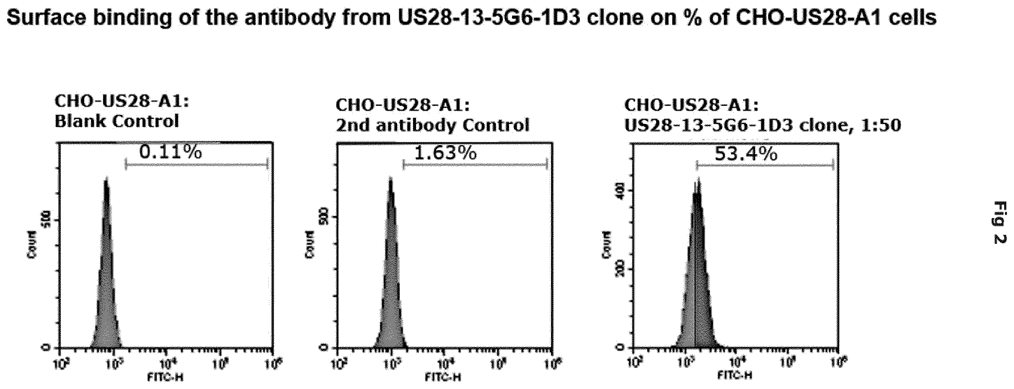

The positive surface binding of the clone US28-13-5G6-1D3 on CHO-US28-A1 cells was further validated by gradient dilution (1:20, 1:50, 1:200, expressed as w/v) showing direct correlation for the antibody binding (FIG. 3). The surface binding to CHO control cells was confirmed to be low in several experiments (>15-fold less binding), indicating that the binding of clone US28-13-5G6-1D3 Ab to the surface of US28 expressing CHO cells was highly specific (FIG. 4).

The original ELISA analysis showed equal qualitative binding affinity of the US28-13-5G6 antibody clones on both Peptide-1 (US28 ECD3 genotype 4N) and Peptide-2 (US28 ECD3 genotype 4D) (FIG. 5). These results indicate that the generated US28-13-5G6-1D3 antibody clone can bind well and specifically on the surface of the HCMV US28 protein overexpressing CHO cells, and that the binding of the US28-13-5G6-1D3 antibody to its target is HCMV strain agnostic.

### Creation and Validation of the US28-13-5G6-1D3 rAb

To enable recombinant production of US28-13-5G6-1D3 antibody, the US28-13-5G6-1D3 encoding hybridoma clone was sequenced. The following sequences of the US28-13-5G6-1D3 antibody were identified, with reference to the sequence identification numbers (“SEQ ID Nos”) described below:

| | | DNA Sequence | Protein Sequence |
|---|---|---|---|
| Complete heavy chain | | SEQ ID NO: 34 | SEQ ID NO: 20 |
| (including leader sequence) | | | |
| Complete light chain | | SEQ ID NO: 35 | SEQ ID NO: 21 |
| (including leader sequence) | | | |
| V_{H} chain | Immature | SEQ ID NO: 25 | SEQ ID NO: 11 |
| | Mature | SEQ ID NO: 26 | SEQ ID NO: 12 |
| V_{L} chain | Immature | SEQ ID NO: 31 | SEQ ID NO: 17 |
| | Mature | SEQ ID NO: 32 | SEQ ID NO: 18 |

| | | |
|---|---|---|
| V_{H}-CDR1 | SEQ ID NO: 22 | SEQ ID NO: 8 |
| V_{H}-CDR2 | SEQ ID NO: 23 | SEQ ID NO: 9 |
| V_{H}-CDR3 | SEQ ID NO: 24 | SEQ ID NO: 10 |
| V_{L}-CDR1 | SEQ ID NO: 28 | SEQ ID NO: 14 |
| V_{L}-CDR2 | SEQ ID NO: 29 | SEQ ID NO: 15 |
| V_{L}-CDR3 | SEQ ID NO: 30 | SEQ ID NO: 16 |

Sequences corresponding to the above-noted SEQ ID Nos are given in the section of this application entitled “Sequences”, which follows these Examples.

The sequencing analysis demonstrated that the subtype of US28-13-5G6-1D3 is most likely IgG1 kappa. The US28-13-5G6-1D3 DNA sequences were inserted in two cloning vectors and transfected into the HEK293-F cell line for recombinant production. Binding of the resulting recombinant antibody product US28-13-5G6-1D3 rAb, was tested for Bio-Peptides 1 and 2 by qualitative ELISA showing again equal, strong and high binding affinity to both US28 ECD3 genetic variants confirming that the recombinant antibody also shows HCMV strain agnostic properties (FIG. 6).

The surface binding of 13-5G6-1D3 rAb to the CHO-US28-A1 cells was thereafter validated and compared against antibodies from other candidate hybridoma clones (13-5C6-1B5, 13-5G6-1D3 culture, 13-4C10-ID8, 14-1H3-1A6, 14-2C2-1A7, 14-2E3-1A12, 14-4E4-1B3) (data not shown). Similar to the earlier test results with the antibody from 13-5G6-1D3 clone, the results show most binding of the 13-5G6-1D3 rAb to CHO-US28-A1 cells.

Further FACS validation of surface binding of the 13-5G6-1D3 rAb to CHO-US28-A1 cells, and to respective control CHO cells, in 1:10, 1:50 and 1:100 dilutions (w/v) showed that the 13-5G6-1D rAb binds on the surface of 16.55% of the US28 expressing cells and that the optimal staining dilution that we tested for the 13-5G6-1D3 rAb is 1:50 (w/v). The surface binding of the 13-5G6-1D rAb to control CHO cells was measured at 1:10 dilution (w/v), this being a high concentration that should enhance the detection of any non-specific binding, and yet was shown to bind to only to 0.77% of the control CHO cells, after removing the binding to blank and anti-mouse IgG-Alexa 488 secondary Ab controls (FIG. 7). These results showing >21 times higher surface binding are fully in line with the other specificity results that we have obtained with the 13-5G6-1D3 Ab clone on US28 expressing CHO cells when compared with the control CHO cells (FIG. 4). Several of the other antibody clones that we generated against ECD3 of US28 showed also great specificity for the US28 expressing cells (ranging from a specificity factor of around 11-fold to 26-fold greater binding to US28 expressing CHO cells compared to the level of binding observed against US28-negative control CHO cells), therefore confirming the ECD3 peptides as a specific target for the US28 binding antibodies (Table 2).

### Creation and Validation of the ECD1-Binding Molecules

To enable recombinant production of 4H3C3, 7B1F3 and 2F5B11 antibodies (selected as exemplary ECD1-binding molecules), respective encoding hybridoma clones was sequenced.

The following sequences of the 4H3C3 antibody were identified, with reference to the SEQ ID Nos described below:

| 4H3C3 | | DNA Sequence | Protein Sequence |
|---|---|---|---|
| Complete heavy chain | | SEQ ID NO: 178 | SEQ ID NO: 180 |
| (including leader sequence) | | | |
| Complete light chain | | SEQ ID NO: 179 | SEQ ID NO: 181 |
| (including leader sequence) | | | |
| V_{H} chain | Immature | SEQ ID NO: 182 | SEQ ID NO: 186 |
| | Mature | SEQ ID NO: 183 | SEQ ID NO: 187 |
| V_{L} chain | Immature | SEQ ID NO: 184 | SEQ ID NO: 188 |
| | Mature | SEQ ID NO: 185 | SEQ ID NO: 189 |

| | | |
|---|---|---|
| V_{H}-CDR1 | SEQ ID NO: 190 | SEQ ID NO: 196 |
| V_{H}-CDR2 | SEQ ID NO: 191 | SEQ ID NO: 197 |
| V_{H}-CDR3 | SEQ ID NO: 192 | SEQ ID NO: 198 |
| V_{L}-CDR1 | SEQ ID NO: 193 | SEQ ID NO: 199 |
| V_{L}-CDR2 | SEQ ID NO: 194 | SEQ ID NO: 200 |
| V_{L}-CDR3 | SEQ ID NO: 195 | SEQ ID NO: 201 |

The following sequences of the 7B1F3 antibody were identified, with reference to the SEQ ID Nos described below:

| 7B1F3 | | DNA Sequence | Protein Sequence |
|---|---|---|---|
| Complete heavy chain | | SEQ ID NO: 202 | SEQ ID NO: 204 |
| (including leader sequence) | | | |
| Complete light chain | | SEQ ID NO: 203 | SEQ ID NO: 205 |
| (including leader sequence) | | | |
| V_{H} chain | Immature | SEQ ID NO: 206 | SEQ ID NO: 210 |
| | Mature | SEQ ID NO: 207 | SEQ ID NO: 211 |
| V_{L} chain | Immature | SEQ ID NO: 208 | SEQ ID NO: 212 |
| | Mature | SEQ ID NO: 209 | SEQ ID NO: 213 |

| | | |
|---|---|---|
| V_{H}-CDR1 | SEQ ID NO: 214 | SEQ ID NO: 219 |
| V_{H}-CDR2 | SEQ ID NO: 215 | SEQ ID NO: 220 |
| V_{H}-CDR3 | SEQ ID NO: 216 | SEQ ID NO: 221 |
| V_{L}-CDR1 | SEQ ID NO: 251 | SEQ ID NO: 199 |
| V_{L}-CDR2 | SEQ ID NO: 217 | SEQ ID NO: 222 |
| V_{L}-CDR3 | SEQ ID NO: 218 | SEQ ID NO: 223 |

The following sequences of the 2F5B11 antibody were identified, with reference to the SEQ ID Nos described below:

| 2F5B11 | | DNA Sequence | Protein Sequence |
|---|---|---|---|
| Complete heavy chain | | SEQ ID NO: 224 | SEQ ID NO: 226 |
| (including leader sequence) | | | |
| Complete light chain | | SEQ ID NO: 225 | SEQ ID NO: 227 |
| (including leader sequence) | | | |
| V_{H} chain | Immature | SEQ ID NO: 228 | SEQ ID NO: 232 |
| | Mature | SEQ ID NO: 229 | SEQ ID NO: 233 |
| V_{L} chain | Immature | SEQ ID NO: 230 | SEQ ID NO: 234 |
| | Mature | SEQ ID NO: 231 | SEQ ID NO: 235 |

| | | |
|---|---|---|
| V_{H}-CDR1 | SEQ ID NO: 236 | SEQ ID NO: 242 |
| V_{H}-CDR2 | SEQ ID NO: 237 | SEQ ID NO: 243 |
| V_{H}-CDR3 | SEQ ID NO: 238 | SEQ ID NO: 244 |
| V_{L}-CDR1 | SEQ ID NO: 239 | SEQ ID NO: 199 |
| V_{L}-CDR2 | SEQ ID NO: 240 | SEQ ID NO: 245 |
| V_{L}-CDR3 | SEQ ID NO: 241 | SEQ ID NO: 246 |

Sequences corresponding to the above-noted SEQ ID Nos are given in the section of this application entitled “Sequences”, which follows these Examples. An additional subclone, 2G2B3 was also further analysed with the sequenced subclones.

The sequencing analysis demonstrated that the subtypes of 4H3C3, 7B1F3 and 2F5B11 are most likely IgG1 kappa. The 4H3C3, 7B1F3 and 2F5B11 DNA sequences were respectively inserted in two cloning vectors and transfected into the HEK293-F cell line for recombinant production. Binding of the clone antibody products 4H3C3, 7B1F3 and 2F5B11 rAbs, was tested for Bio-Peptide-N by qualitative ELISA showing again equal, strong and high binding affinity to US28.

The surface binding of 4H3C3, 7B1F3 and 2F5B11 clone Abs to the CHO-US28-A1 cells was thereafter validated. Similar to the earlier test results with the antibody from 4H3C3, 7B1F3 and 2F5B11 subclones, the results show most binding of the 4H3C3, 7B1F3 and 2F5B11 rAbs to CHO-US28-A1 cells.

### Binding of US28-13-5G6-1D3 rAb to HCMV Infected MRC-5 Cells

We then studied whether the US28-13-5G6-1D3 rAb was able to bind to the surface of HCMV infected cells specifically. HCMV is known to enter, replicate and cause lytic infections in human fibroblasts [3]. We therefore infected human MRC-5 cells with the HCMV Ad169 laboratory strain at 5 m.o.i. and harvested the cells at day 4 p.i. (96 hpi). The infected cells showed typical signs of lytic HCMV infection (cytomegalo characterized by flat, swollen cells) in a majority of the cells as observed by microscopy.

The surface binding of the US28-13-5G6-1D rAb to HCMV Ad169 infected cells was compared with infected cells stained with the appropriate IgG isotype control as well as with uninfected, cultured MRC-5 cells (Mock) by using flowcytometry analysis. The results showed specific binding of US28-13-5G6-1D rAb on the surface of the HCMV Ad169 infected MRC-5 cells but not on the Mock cells (FIGS. 8-9).

To control that the 13-5G6-1D rAb was binding to the surface of the HCMV infected cell population, another set of HCMV Ad169 infected and Mock cells were permeabilized and stained with the commercial antibody MAB810X against the HCMV Major Immediate Early (IE) antigen, which is an intracellular protein known to be expressed early during the lytic HCMV infection [3]. The MAB810X antibody stained the same HCMV infected MRC-5 cell population as the US28-13-5G6-1D3 rAb in FACS analysis, which was not observed for non-infected cells for either antibodies, confirming that the surface binding demonstrated for the US28-13-5G6-1D3 rAb was specific to HCMV infected cells (FIGS. 8A and B).

Non-specific binding of the US28-13-5G6-1D3 rAb was tested by using non-immune serum made in mouse (IgG isotype control) instead of the primary antibody prior to incubation with Goat anti-mouse IgG-Alexa 488. The results showed a shift in staining of the HCMV infected MRC-5 cells by the US28-13-5G6-1D3 rAb compared with the isotype control, while staining of uninfected cells (Mock) were similar to the IgG isotype control. Taken together, these results show that the US28-13-5G6-1D3 rAb binds specifically (˜16-fold greater specificity) to the surface of the HCMV infected cells as compared with the uninfected cells (FIG. 9).

It is noted that HCMV Ad169 laboratory strain encodes a 4D variant of US28 (SEQ ID NO: 42), in that it presents D at the 4^{th} amino acid of the ECD3 sequence; in contrast to the 4N variant encoded by DB strain which was the sequence used in creating the CHO-US28-A1 cells discussed in the earlier experiments. The binding of US28-13-5G6-1D3 to both the 4D and 4N variants of US28, as demonstrated in the present examples, is a clear indication of the strain agnostic binding properties provided.

### US28-13-5G6-1D3 Binding on Human PBMCs

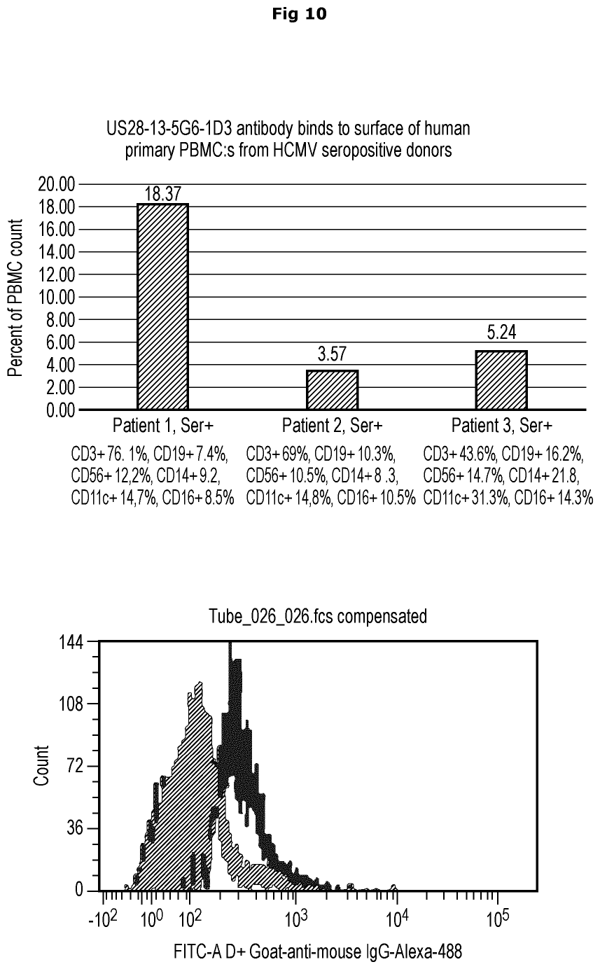

Life-long latent HCMV carriage in CD34+ progenitors and their derivatives, including granulocyte-macrophage progenitors and CD14+ monocytes, provide a reservoir of HCMV that can be reactivated in seropositive individuals [4]. We next investigated whether the US28-13-5G6-1D3 rAb could bind to primary PBMCs from three HCMV seropositive individuals (donors 1-3) by using the same flowcytometry protocol as for our earlier studies. The results showed surface binding by the US28-13-5G6-1D3 rAb on 18.37%, 3.57% and 5.25% of the PBMC cells from the three donors, respectively (FIG. 10). Of the studied PBMCs, only the CD11+, CD14+ and CD16+ positive cells can be carriers of the HCMV, and these cell markers can partly overlap in different mononuclear cells. Depending on the person and situation, the latently infected cell population adds up to about 15% but can vary. Consequently, the observed surface binding of the US28-13-5G6-1D3 rAb on 18.37%, 3.57% and 5.25% of the total PBMCs in donors 1, 2 and 3, respectively, demonstrates binding to a substantial proportion of the PBMCs that can be HCMV carriers. These results are not related to the subtypes of the PBMCs, but indicate that the US28-13-5G6-1D3 rAb can bind to the surface of latently HCMV infected cells.

### Commercial US28 Antibody

The commercial polyclonal US28 Ab was purchased to compare their binding properties with the US28-13-5G6-1D3 Ab.

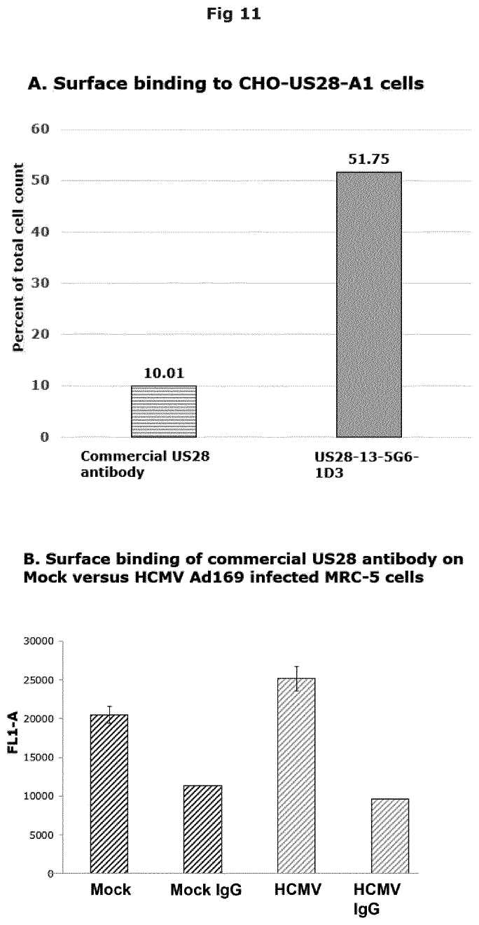

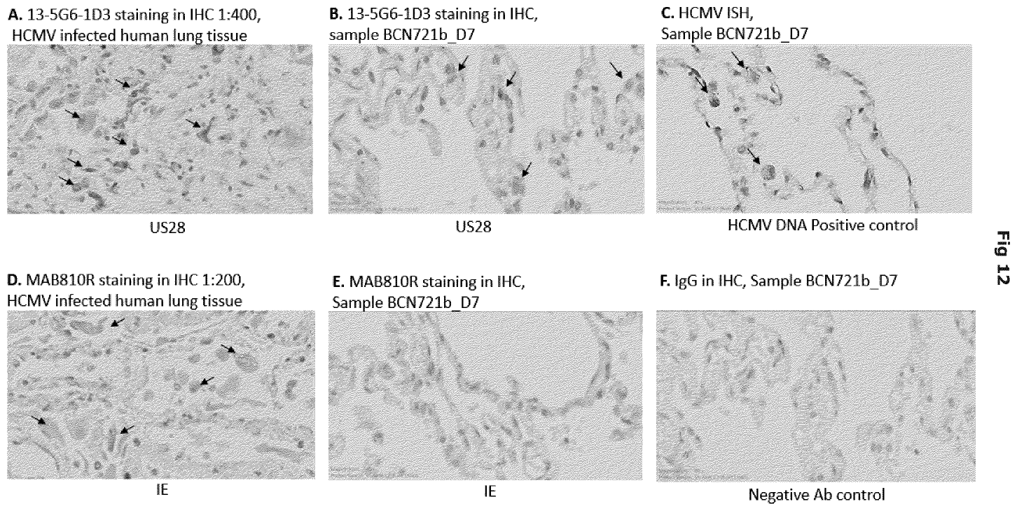

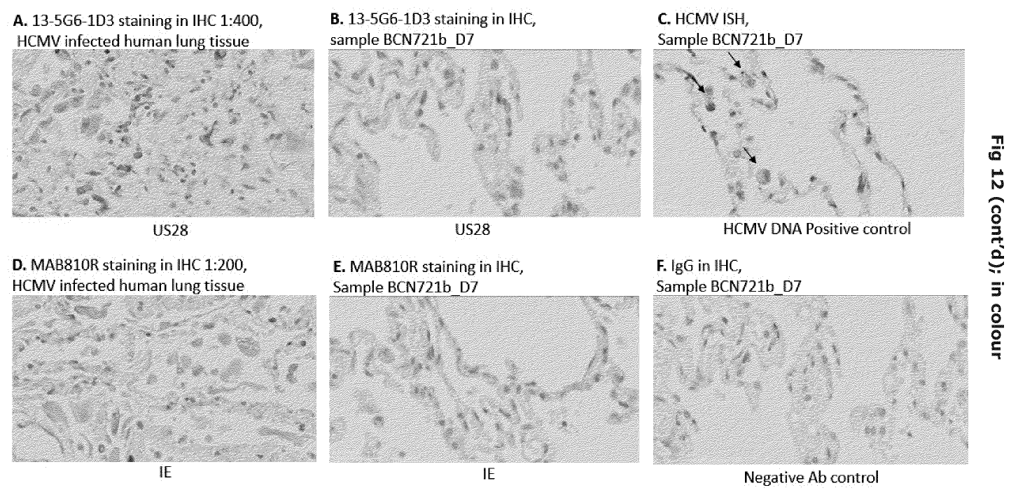

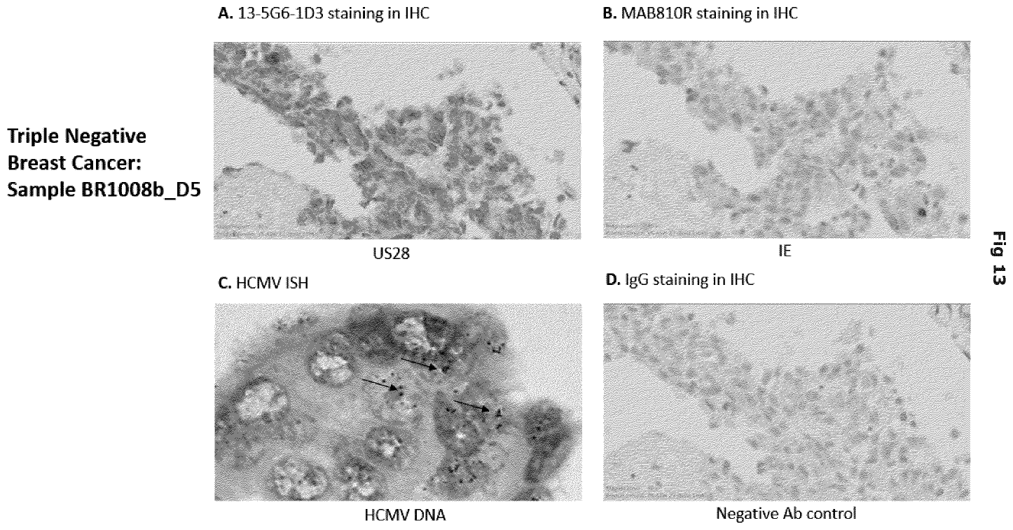

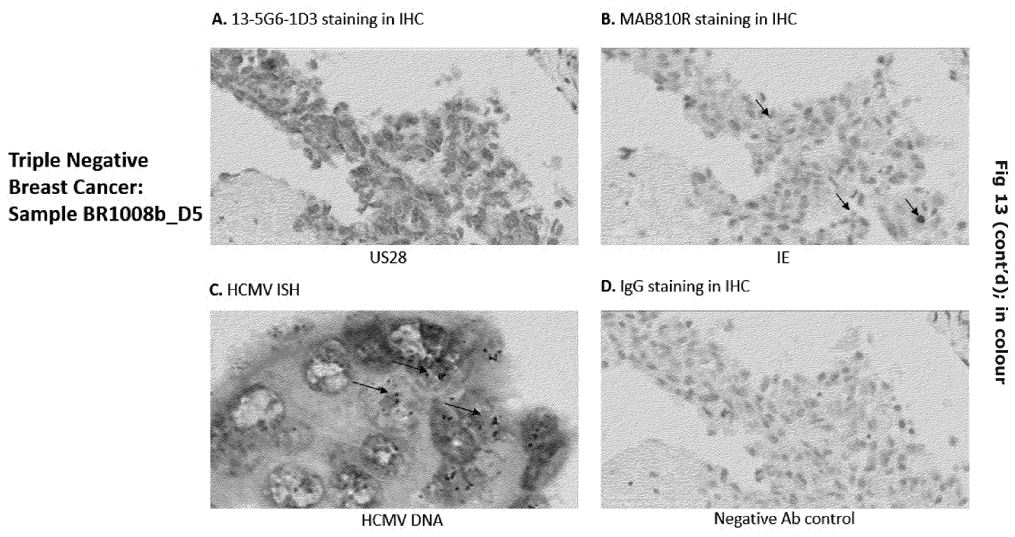

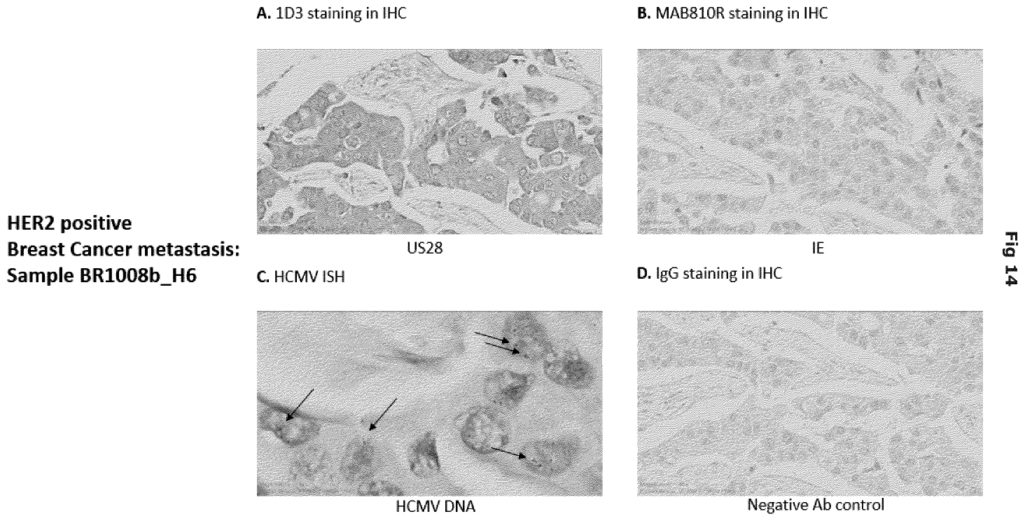

Binding of the commercial US28 polyclonal Ab to CHO-US28-A1 cells was tested and compared with the binding of US28-13-5G6-1D3 rAb to the same cells. The US28-13-5G6-1D3 antibody bound to more than 50% of the CHO-US28-A1 cells, while the commercial US28 Ab only stained 10% of the cells in the same experiment (FIG. 11A). We then tested the commercial US28 Ab on HCMV Ad169 infected MRC-5 cells, where it showed specific binding to the HCMV infected population. However, this antibody also stained non-infected Mock cells about two-fold compared to the IgG isotype control indicating unspecific binding on the surface of non-infected MRC-5 cells (FIG. 11B). In addition, the binding of the commercial US28 antibody on human primary PBMCs was >80% in all three tested samples indicating unspecific binding to this cell population as the latently infected cells add up to about 15% (data not shown). Thus, part of the specific binding effect of the commercial US28 antibody observed on the HCMV Ad169 infected MRC-5 cells may arise from this unspecific surface binding component.

Comparison with Monovalent and Bivalent VUN100

WO 2019/151865, as also reported in the equivalent journal article De Groof et al, 2019 [5], describes single heavy chain variable domain antibodies (VHH) against US28, exemplified by a particular VHH referred to as VUN100. VUN100 (the sequence of which is provided as SEQ ID NO: 60 of the present application) was shown to bind to a discontinuous epitope, which comprise multiple binding positions in the N-terminal extracellular region of US28 (positions 1-37, also referred to herein as ECD1), and further influenced by the presence of the third extracellular loop (ECL3, positions 250-273, also referred to herein as ECD4) of US28, as discussed in Example 3 of WO 2019/151865 (page 36, lines 11-32) and the legend to FIG. 2 of De Groof et al, 2019 [5]. Surface binding of the VUN100 Ab to the of HEK293-F Mock cells (˜20%) with a specificity score of 4 was reported by De Groof et al., 2019 [5] in their supporting information, FIG. 51. A thereof (and summarised in Table 2 of the present application). The apparent ability to distinguish between US28-expressing and US28-negative cells, with a specificity score of only around 4, is sub-optimal in comparison to the consistently higher levels of specificity scores obtained using antibodies generated against ECD3 of US28, including antibodies 13-5G6-1D3, 13-5C6-1B5 and 14-1H3-1A6, which consistently demonstrated specificity scores in the range of 11-26 (Table 2), even when high antibody concentrations were selected to maximise any non-specific binding, and with even greater specificity scores demonstrated using more commercially-relevant (lower) antibody concentrations (FIG. 7).

The low specificity scores reported for VUN100 raises concerns about its ability to provide specifically targeted effects to HCMV-infected cells, whilst avoiding off-target side effects in healthy cells. The provision of highly-specific binding molecules against ECD3 of US28, which substantially avoid unacceptable levels of off-target binding in healthy cells, is a particular focus of the present invention, in the context of directing cytotoxic activities with high specificity to HCMV-infected cells, such as by using BiTEs and/or CAR-T cells comprising the US28-binding activity of the binding molecules provided herein. The properties reported for VUN100 indicate its lack of suitability for directing cytotoxic activities with high specific towards HCMV-infected cells. It also raises concerns about the ability of VUN100 to be useful in the context of reliably identifying US28-expressing cells (in particular, HCMV-infected cells) in assays, including diagnostic assays, such as for use in immunohistochemistry (IHC), in contrast to the highly specific binding molecules of the present invention.

In WO 2019/151865, and the equivalent journal article De Groof et al, 2019, Mol. Pharmaceutics, 16: 3145-3156, the authors referred to VUN100 and indicated an ability to specifically detect US28 in GBM tissues and inhibit ligand-dependent and constitutive US28 activity, and to consequently impair US28-dependent GBM growth in vitro and in vivo in an orthotopic xenograft model.

However, in order to make use of US28 as a therapeutic target, particularly for the purpose of directing cytotoxic activities to cells showing surface expression of US28, it is important to overcome the most important obstacles, as previously described, not only including the relatively high levels of non-specific binding activity that has been reported for the art-known VUN100 molecule, but also to provide US28-binding molecules that overcome obstacles of strain diversity, viral mutations, mutagenic drift and the ability of the virus to hide from the immune system during viral latency.

Therefore, a comparison has been made between the binding molecules of the present invention (such as 13-5G6-1D3) which bind to ECD3 of US28, and the VUN100 Ab of WO 2019/151865 and De Groof et al, 2019 (supra) and/or the VUN100b bivalent molecule of De Groof et al, 2020 (supra), which has been reported to bind to a discontinuous epitope present within the N-terminus and ECD4 of US28.

The comparison was conducted in CHO cells recombinantly expressing US28 sequences from HCMV strain DB (as a representative 4N-variant) or HCMV strain TB40/E (as a representative 4D-variant), and using non-recombinant CHO cells as a control. As noted above, the applicant has identified that HCMV strains can be categorised by reference to their ECD3 sequence, and generally fall into the scope of being a 4N- or 4D-variant (this being approximately a 10%/90% split, respectively), and so the DB and TB40/E strains exemplified are representative of a number of further strains that are within these two groupings. Therefore, showing cross-reactivity for both 4N- and 4D-variants is indicative of strain agnostic binding for a wide range of HCMV strains.

FACS analysis was performed (as described above) for each of the infected cell lines with the following primary antibodies: (i) 13-5G6-1D3; (ii) monovalent (abbreviated as “Mv”) VUN100; and (iii) bivalent VUN100 (abbreviated to “BvVUN100” and also referred to as VUN100b). Cell binding is represented as a percentage of cells in the population that are bound by the antibody.

A low percentage of binding was observed for the CHO negative control, with monovalent VUN100 showing 2.25% background binding, bivalent VUN100 showing 1.06% background binding, and 13-5G6-ID3 (abbreviated as “1D3” in the figure) showing the lowest background binding of 0.88% (FIG. 21A). These data demonstrate that the exemplary ECD3-binding antibody, 13-5G6-1D3, has lower background binding to non-infected CHO cells than the VUN100 antibodies.

In CHO cells expressing US28 encoded by the DB strain of HCMV (an exemplary 4N-variant), an increase in binding for all test antibodies was observed, with monovalent VUN100 binding to 6.91% of the population, bivalent VUN100 binding to 9.24% of the population, and 13-5G6-1D3 binding to 16.65% of the population (FIG. 21A). This shows that, in addition to having lower background binding than either monovalent or bivalent VUN100, 13-5G6-1D3 also has substantially higher binding to CHO cells expressing the DB form of US28. When expressed as % increase in binding, between CHO control cells and CHO cells expressing the DB form of US28, this being an indication of markedly improved specificity, 13-5G6-1D3 (FIG. 21B). These data demonstrate that the exemplary ECD3-binding antibody, 13-5G6-1D3, has improved specific binding to CHO cells infected with a 4N-variant strain of HCMV.

Similarly, when comparing binding in CHO cells expressing the TB40/E form of US28 (an exemplary 4D-variant) to the binding to CHO control cells, the data clearly show higher binding of the exemplary ECD3-binding antibody, 13-5G6-1D3, to CHO cells expressing the TB40/E form of US28 (FIG. 21A) and greatly improved specificity (FIG. 21B) both monovalent and bivalent VUN100.

These data also give an indication of relative levels of strain-agnostic binding activity.

One can compare the level of binding in CHO cells expressing DB form of US28 (an exemplary 4N-variant in ECD3) and the TB40/E form of US28 (an exemplary 4D-variant in ECD3) of the different antibodies in the panel. As shown in FIG. 21A, decreased binding to the 4D-variant was observed compared with the 4N-variant for monovalent VUN100, down from 6.91% to 4.33% of the population. Likewise, bivalent VUN100 showed decreased binding, down from 9.24% to 5.2% of the population. These results are in concordance with the findings in WO 2019/151865. Interestingly, however, the exemplary ECD3-binding antibody, 13-5G6-1D3, has further improved specific binding in comparison with the 4N-variant strain, increased slightly from 16.65% to 18.25% of the population.

FIG. 21C shows the % change in absolute levels of binding with the cell population, for the different antibodies in the test panel, between binding to CHO cells expressing the DB and TB40/E forms of US28. From these data, it is clear that there are substantial differences in the binding capability of both monovalent and bivalent VUN100 (greater than 35% and greater than 40%, respectively) depending on the strain of HCMV from which of US28 sequence is derived. In contrast, the HCMV strain identity from which the US28 protein is obtained impacts on the binding of the exemplary ECD3-binding antibody, 13-5G6-1D3, to a much lesser extent, the difference being less than 10%.

These data can also be analysed to determine the percentage retention of binding activity to US28, when comparing US28 derived from these difference strains of HCMV. FIG. 21D shows that the exemplary ECD3-binding antibody, 13-5G6-1D3, retains essentially 100% binding activity between these strains, whereas VUN100 monovalent and bivalent forms retain only about, or less than, 60% (that is, a loss of about or greater than 40%) depending on the HCMV strain identity from which the US28 protein is obtained.

These differences in binding capability dependent on the strain of HCMV from which US28 is obtained is also clearly illustrated in FIG. 21E, in which the ability of each of monovalent and bivalent VUN100 to bind to US28 from each tested HCMV strain is calculated, based on a value normalised against the binding of 13-5G6-1D3 to, respectively, the same form of US28. When normalised in this way, the strain-dependent variability in the binding each of monovalent and bivalent VUN100, compared to that observed for 13-5G6-1D3, is clearly visible.

These data demonstrate that while the monovalent and bivalent VUN100 anti-US28 antibodies have a binding activity for US28 that is heavily influenced by the strain of HCMV that encodes US28, in contrast an exemplary ECD3-binding antibody of the present invention, 13-5G6-1D3, is effectively strain agnostic, with high levels of binding for both 4N- and 4D-variants of HCMV.

These data show further that a strain agnostic epitopic sequence (“SAES” exists within US28 that ECD3-binding molecules of the present invention can exploit to retain high binding specificity for the variety different strains of HCMV. Accordingly, binding molecules of the present invention target an advantageous epitope, i.e. a SAES within ECD3 of US28, that has not previously been targeted. In contrast, the VUN100 molecules of the art, which bind to a discontinuous epitope within the N-terminal region and ECD4, have comparatively higher background (off-target) binding, substantially lower specificity, and display HMCV strain-dependent binding properties.

These comparative results demonstrate that antibodies of the present invention, which bind to the SAES within ECD3 of US28, as exemplified by US28-13-5G6-1D3 rAb, shows a high degree of US28-specific surface binding properties on all cell lines and cells tested, whereas both the reference antibodies: the commercial polyclonal US28 Ab and VUN100 Ab monoclonal antibody, show a high degree of unspecific binding on the surface of the US28 negative control cells. Thus, antibodies that have been raised against ECD3 of US28 in accordance with the protocol described herein, as exemplified in particular by the US28-13-5G6-1D3 rAb and the other anti-ECD3 antibodies obtained by the process described in the present application, are according to our knowledge the only currently existing antibodies that are able to bind on the surface of the US28-expressing, HCMV infected, cells in a manner that is both highly specific and yet also strain agnostic.

### Binding to an Artificially Created Mutated Strain of US28

US28 has a number of known points within its sequence that can be mutated (see Table 3). Furthermore, viruses such as HCMV are known to mutate to adapt to therapy. For example, Valganciclovir is susceptible to the virus developing resistance through mutations (Boivin et al., 2012; Göhring et al., 2015; and Jung et al., 2019). Therefore, a mutated version of the US28 was prepared that includes mutations at all known points within its sequence (referred to herein as the “mutated” form), in order to simulate an extreme form of possible future genetic drift within HCMV strains. The sequence is provided herein as SEQ ID NO: 173, and has the following mutations, compared to the sequence of US28 as encoded by HCMV strain DB (SEQ ID No: 5): P3Q, T7del (i.e. deletion), A8T, E18L, A19G, T21A, P22L, V24T, F25L, V35I, N170D, V250L, F265S, R267K.

CHO cells engineered to express the mutant form (“CHO-mutated strain”) were compared for binding between an exemplary ECD3-binding molecule, 1D3, and the VUN100 molecules that do not bind to ECD3 (see FIG. 21A).

Interestingly, no binding was detected to the mutated strain above that of the CHO negative control by observed for monovalent VUN100 (1.41% binding to the mutated strain, compared with 2.25% binding for the control CHO cells in these experiments), indicating that monovalent VUN100 actually binds more strongly to off-target cells than to US28-expressing cells. This is further illustrated in FIG. 21B, which shows a specificity of less than 1 for monovalent VUN100 for the CHO-mutated strain (i.e. it binds less to mutated US28-expressing cells than to control cells).

Bivalent VUN100 exhibited very low binding of 2.58% to cells expressing the mutated form of US28, which is only an increase of 1.52% above that of the control CHO cells (FIG. 21A), this corresponding to only a 2.43-fold increase in binding to cells expressing the mutated strain form of US28 compared to control CHO cells (FIG. 21B).

In contrast, an exemplary ECD3-binding molecule of the present invention, 13-5G6-1D3 (“1D3”), retained high binding to cells expressing the mutated US28 form at 12.93% compared to 0.88% of control cells (FIG. 21A), this being almost a 15-fold greater level of binding to the cells expressing the US28 mutated form, compared to control cells (FIG. 21B).

These data demonstrate that, whereas the VUN100 molecules of the prior art (which bind to an discontinuous epitope with the N-terminal region and ECD4 of US28) can be highly susceptible to changes in binding properties and specificity, depending on viral mutation (e.g. when evolving to develop resistance), the ECD3-binding molecules of the present invention (such as 1D3) have binding that is directed to the identified strain agnostic epitopic sequence (“SAES”), and this allows the anti-ECD3 binding molecules of the present invention to maintain high levels of specific binding to further mutated strains of US28. Therefore, based on these data, it is less likely that HCMV will develop resistance to ECD3-binding molecules through mutation than it will, for example, to the VUN100 antibodies as described in WO 2019/151865, De Groof et al, 2019, supra, and De Groof et al, 2020, supra. This is an additional advantage for the ECD3-binding molecules of the present invention over that of other HCMV therapies such as Valganciclovir.

### Example 2

Additional ECD3 antibodies bind to 4N- and 4D-variants of HCMV In view of the finding in Example 1, we hypothesise that the process of generating and selecting antibodies, and other binding molecules, against ECD3 of US28 in the above manner using the 4N- and 4D-Bio-Peptides, results in the provision of beneficial binding properties, including strain agnostic binding due to the use of the identified strain agnostic epitopic sequence (“SAES”) within ECD3.

In order to confirm this hypothesis, further subclones were generated using the same methods as previously described, and subsequently tested for binding to both peptide variants by ELISA (as described above, see Assay 1). As before, the peptide sequences used in this ELISA assay were Bio-Peptide-1, having the sequence TKKNNQCMTDYDYLEVS (SEQ ID NO: 6, corresponding to the 4N variant of ECD3) and Bio-Peptide-2, having the sequence of TKKDNQCMTDYDYLEVS (SEQ ID NO: 7, corresponding to the 4D variant of ECD3).

28 subclones were tested in total that were generated with specificity to ECD3 of US28. Details of the clones from which the subclones were derived can be seen in Table 4. The “13” and “14” indicate Mouse 13 and Mouse 14 as previously described. The subclones can therefore be fully characterised as, for example, 13-1C10-1A2 by tagging the subclone identity to the end of the respective clone identity and excluding the “US28”, which merely denotes that the clones are raised for specificity to US28.

TABLE 4
Clones and subclones of ECD3 binding molecules.

| Clone Identity | Subclone identity |
|---|---|
| US28-13-1C10 | 1A2, 1A8, 1B9, 1B11, 1C10, 1D3, 1G8, 1G9 |
| US28-14-1H3 | 1A10, 1C10, 1C11, 1C12, 1D2, 1E1 |
| US28-14-2C2 | 1A2, 1A3, 1G4, 1G9, 1G11 |
| US28-14-4E4 | 1A1, 1A4, 1E6, 1E8 |
| US28-14-4F11 | 1A1, 1A2, 1A3, 1A4 |
| US28-14-5H11 | 1H6 |

The full ECD3 subclone identities are listed in Table 5A with the absorbance values for each subclone numerically represented in corresponding Table 5B. Both of these tables are representative of the 96-well plate layout for the experiment, and so a cell (representative of a well in the 96-well plate) for Table 5A that denotes the antibody subclone can be matched up with the corresponding cell for Table 5B. The blank wells were coated with the respective peptides and treated with secondary antibody in the absence of a primary antibody.

High absorbance values were observed for all 28 antibody subclones to both Bio-Peptide-1 and Bio-Peptide-2, which demonstrates that a number of antibodies of varying sequences can be raised against ECD3 that will share the beneficial property of strain agonistic binding to 4N- and 4D-variants of HCMV.

The full ECD1 subclone identities are listed in Table 5C with the absorbance values for each subclone numerically represented. The blank wells were coated with the respective peptides and treated with secondary antibody in the absence of a primary antibody.

High absorbance values were observed for all 29 antibody subclones to Bio-Peptide-N, which demonstrates that a number of antibodies of varying sequences can be raised against the highly conserved epitope of ECD1 that will share the likely beneficial property of strain agonistic binding to variants of HCMV.

TABLE 5C
ECD1 antibody clones and absorbance values for
the additional 29 clones tested for binding
to the conserved N-terminal epitope of HCMV.

| | Coating: | |
|---|---|---|
| | Peptide- | Coating: |
| Clone ID | N-BSA | BSA |
| 1E10F4 | 2.5076 | 0.0414 |
| 2E10B7 | 2.686 | 0.079 |
| 2F5B11 | 2.8039 | 0.0508 |
| 2G2B3 | 2.8319 | 0.0015 |
| 3E1D7 | 2.7848 | 0.066 |
| 4A11A11 | 2.7409 | 0.0132 |
| 4B7E1 | 2.6664 | 0.0481 |
| 4D10B6 | 2.6751 | 0.0194 |
| 4E1B10 | 2.5898 | 0.0652 |
| 4H3C3 | 2.7481 | 0.0164 |
| 4H9B5 | 2.739 | 0.0326 |
| 5D6H11 | 2.8319 | 0.0479 |
| 5E1E4 | 2.7131 | 0.0051 |
| 5E8F7 | 2.7989 | 0.0634 |
| 5F10H11 | 2.7027 | 0.0424 |
| 6A1B4 | 2.7113 | 0.0425 |
| 6B2B5 | 2.5635 | 0.0279 |
| 6B3C10 | 2.6402 | 0.0255 |
| 6D5G3 | 2.8039 | 0.0211 |
| 6G3D6 | 2.819 | 0.0291 |
| 7A2C10 | 2.9445 | 0.0319 |
| 7B1F3 | 2.7989 | 0.0474 |
| 7B11D6 | 2.4905 | 0.0162 |
| 7D12D11 | 2.6529 | 0.0012 |
| 7E11F7 | 2.5726 | 0.0197 |
| 9G3B9 | 2.674 | 0.0008 |
| 10F9B1 | 2.6189 | 0.0977 |
| 10G1D5 | 2.6867 | 0.0484 |
| 10G6C9 | 2.6987 | 0.0626 |
| Blank | 0.056 | 0.0356 |
| (negative | | |
| cells) | | |
| Positive | 2.4711 | 0.0774 |
| serum | | |

TABLE 5A
ECD3 antibody clones key for the additional 28 clones tested for binding to 4N- and 4D-variants of HCMV.

| | 1 | 2 | 3 | 4 | 5 | 6 | 7 | 8 | 9 | 10 | 11 | 12 |
|---|---|---|---|---|---|---|---|---|---|---|---|---|
| Peptide-1 | 13-1C10- | 14-2C2- | 14-2C2- | 13-1C10- | 14-5H11- | 13-1C10- | 14-4E4- | 14-4F11- | 14-4E4- | 14-1H3- | 14-1H3- | 14-4E4- |
| Peptide 2 | 1A8 | 1G4 | 1G9 | 1B9 | 1H6 | 1G8 | 1E6 | 1A1 | 1A1 | 1C10 | 1E1 | 1A4 |
| Peptide-1 | 14-4F11- | 14-1H3- | 13-1C10- | 14-2C2- | 14-1H3- | 14-4E4- | 13-1C10- | 14-2C2- | 14-2C2- | 14-1H3- | 14-1H3- | 14-4F11- |
| Peptide 2 | 1A2 | 1C12 | 1A2 | 1A2 | 1D2 | 1E8 | 1D3 | 1G11 | 1A3 | 1A10 | 1C11 | 1A3 |
| Peptide-1 | 13-1C10- | 14-4F11- | 13-1C10- | 13-1C10- | Blank | | | | | | | |
| Peptide 2 | 1C10 | 1A4 | 1G9 | 1B11 | | | | | | | | |

TABLE 5B
Absorbance values for the additional 28 clones tested
for binding to 4N- and 4D-variants of HCMV.

| | 1 | 2 | 3 | 4 | 5 | 6 | 7 | 8 | 9 | 10 | 11 | 12 |
|---|---|---|---|---|---|---|---|---|---|---|---|---|
| Peptide-1 | 2.921 | 2.951 | 2.830 | 3.097 | 2.921 | 2.880 | 2.880 | 2.717 | 2.921 | 2.983 | 2.907 | 2.454 |
| Peptide 2 | 2.951 | 2.811 | 2.747 | 2.823 | 2.944 | 2.993 | 3.090 | 2.944 | 2.976 | 3.187 | 2.914 | 2.374 |
| Peptide-1 | 2.971 | 2.752 | 2.841 | 2.794 | 3.005 | 2.988 | 3.005 | 2.742 | 3.005 | 3.005 | 3.005 | 2.645 |
| Peptide 2 | 2.902 | 2.871 | 2.789 | 2.488 | 2.765 | 2.871 | 2.661 | 2.534 | 2.765 | 2.842 | 2.765 | 2.482 |
| Peptide-1 | 3.106 | 2.987 | 3.106 | 3.154 | 0.033 | | | | | | | |
| Peptide 2 | 3.029 | 2.922 | 3.065 | 2.330 | 0.032 | | | | | | | |

### Binding of Additional Clones to US28-Expressing Cells

It is considered that a large part of the HCMV pathogenesis is associated with viral latency, which is closely linked to virus ability to escape from the humoral and cellular host immune responses through a number of mechanisms (Manandhar et al., Int J Mol Sci, 2019. 20(15)). One of the most important of such mechanisms include this high, ever-changing genetic diversity of the HCMV. Many CMV genes have been identified as highly variable, including the G protein-coupled receptor US28 where numerous N-terminal polymorphisms have been reported (Goffard et al., Virus Genes, 2006; 33(2):175-81; Arav-Boger et al., J Infect Dis., 2002; 186(8):1057-64).

Our investigations have identified that HCMV strains can be categorised by reference to their ECD3 sequence, and fall into the scope of being a 4N- or 4D-variant (this being approximately a 10%/90% split), respectively. The binding data provided above in Table 5B shows that binding molecules generated against the strain agnostic epitopic sequence (“SAES”) within ECD3 consistently show strain agnostic binding, in contrast to the characteristics of anti-US28 antibodies that bind to other regions of the US28 molecule, such as VUN100 (which is reported to bind to a discontinuous epitope in the N-terminal and ECD4 regions of US28), as demonstrated by the data in FIG. 21 of the present application (as discussed above in Example 1).

Thus, binding molecules of the present invention can be characterised by having stain agnostic binding to US28-expressing cells, wherein the extent of binding and/or degree of binding specificity is substantially unaffected by the strain of HCMV that expresses the US28. This can be important not only in view of the fact that different individuals can be infected with different strains of HCMV, but also since individuals can be simultaneously infected with multiple different strains of HCMV (Gorzer et al., J Virol, 2010, 84(14): 7195-7203; Renzette et al., PloS Pathog, 2011, 7(5): e1001344; Renzette et al., Curr Opin Virol, 2014. 8: 109-15; Renzette et al., Proc Natl Acad Sci USA, 2015, 112(30): E4120-8; Renzette et al., J Virol, 2017, 91(5)). In the context of being infected with different strains of HCMV, it is highly beneficial to provide binding agents that are capable of exerting their binding properties irrespective of the HCMV strain present and thus avoiding the need to categorise the strain of HCMV infection before commencing treatment. In the context of individuals being simultaneously infected with multiple different strains of HCMV, it is highly beneficial to be able to treat all strains simultaneously in the individual; in particular, this can help to avoid the creation of an environment which applies selective pressure to promote the development of infection of any non-bound strains.

It is also known that new host infections give rise to a unique viral strain for each infected individual and generate selection events where a new genotype can become dominant due to the selective pressure of the immune response (Renzette et al., 2011, supra), and it is possible that both viral and host factors can contribute to fostering viral genetic drift during the HCMV infection (Vabret et al., Trends Immunol, 2017, 38(1): 53-65; Christensen & Paludan, Cell Mol Imnunol, 2017, 14(1): 4-13). This is a further context in which the provision of anti-US28 binding molecules with strain agnostic binding is important.

It is for at least these reasons that strain agnostic binding (or, at least, minimising the extent of variability in binding properties of the anti-US28 binding molecules of the present invention, as between different strains of HCMV) is highly preferable.

Binding molecules of the present invention can, additionally or alternatively, be further characterised by binding preferentially to US28-expressing cells compared to the binding of US28-negative cells. Thus, specificity is another important characteristic, along with the ability to retain that specificity in a strain-agnostic manner.

It is also important, at least in some contexts (e.g. in therapeutic contexts in which the binding molecules of the present invention are used to deliver cytotoxic activity against bound cells), that the extent of off-target binding is low in an absolute sense. In such contexts, it may not be adequate only for the binding to be strongly-specific to US28-expressing cells. That is to say, whereas very high levels of binding to US28-expressing cells can be helpful in certain contexts, this may not be acceptable in a therapeutic context if that high level of binding to US28-expressing cells also comes with a relatively high level of off-target binding, even though the relative level of binding specificity (comparing the absolute level of binding to target cells versus off-target cells) may give a favourable ratio. Indeed, in such context, the avoidance of binding to off-target cells (i.e. cells not infected by HCMV) can be an additional characteristic of high importance, allowing the provision of therapeutically-useful molecules that avoid off-target activity. That is to say, binding molecules of the present invention with a low absolute level of binding to US28-negative cells can, in some contexts provide a more therapeutically-relevant activity, as high background binding will risk off-target affects for the binding molecules. For example, with CAR T-cells or BiTEs that selectively kill any cell that is bound, it is critical that the CAR T-cell or BiTE have a low level of background binding to cells negative for the target. Otherwise, the CAR T-cell or BiTE may kill cells other than the intended target.

As noted above, in Example 1 of this application (as further shown in FIG. 21), in comparison to the anti-US28 VUN100 prior art antibody (which has been reported to bind to a discontinuous epitope in the N-terminal and ECD4 regions of US28), an exemplary ECD3-binding molecules of the present invention (13-5G6-1D3), provided substantially lower off-target binding activity, provided markedly improved strain agnostic binding, and also provided higher specificity as well as improved retention of that specificity with different HC:MV strains.

Therefore, the binding properties of the selected further anti-ECD3 binding molecules, were further analysed in comparison to VUN100 and 1D3.

To determine the extent of off-target binding and the extent of binding specificity for US28-expressing cells, the same flow cytometry assay as Assay 2, above, was used for various candidate subclones selected from: (i) the 28 strain agnostic ECD3-binding molecules described above (the selected candidates can be seen in Table 6A), and (ii) the 29 ECD1-binding molecules described above (the selected candidates can be seen in Table 6B), and compared to 1D3-mFc, a form of the exemplary 13-5G6-1D3 that contains a murine Fc receptor, as a positive control, and to a mixture of the Mv and By VUN100 molecules, art known binding molecules for US28, at a ratio of 1:1 in 50 μg/ml as a further comparative control.

TABLE 6A
ECD3 subclones selected for binding
assays to US28-expressing cells.

| Clone identity | Subclone identity |
|---|---|
| US28-13-1C10 | 1B9, 1C10, 1G9 |
| US28-14-1H3 | 1A10, 1C10 |
| US28-14-2C2 | 1G4 |
| US28-14-4E4 | 1A1, 1E8 |

TABLE 6B
ECD1 subclones selected for binding
assays to US28-expressing cells.

| Clone Identity | Subclone identity |
|---|---|
| US28-4-2F5 | B11 |
| US28-4-2G2 | B3 |
| US28-4-4A11 | A11 |
| US28-4-4H3 | C3 |
| US28-4-5D6 | H11 |
| US28-4-5E1 | E4 |
| US28-4-5E8 | F7 |
| US28-4-6G3 | D6 |
| US28-4-7B1 | F3 |
| US28-4-9G3 | B9 |

The selected subclones were tested by flow cytometry for binding to CHO cells engineered to overexpress the DB-US28 (“CHO-US28” cells), in comparison to CHO cells that do not overexpress US28 (“CHO” cells) as a way to measure non-specific binding for the various binding molecules. Background levels were determined using the respective secondary antibodies (anti-mFc-FITC for the subclones, or anti-myc-PE for the VUN100 molecules) with CHO cells and CHO-US28 cells. Given that US28 is expressed on the cell surface, the cells were non-permeabilised for these experiments.

Table 7A and FIG. 22A summarises the percentage of binding for ECD3-binding molecules in flow cytometry in control CHO cells (i.e. not expressing US28), which is indicative of the risk for off-target binding, with the background levels for the secondary antibodies removed. Therefore, the percentage of binding indicated by each condition is off-target binding of the tested binding molecules.

TABLE 7A
Percentage of binding for ECD3-binding molecules
to CHO cells that are negative for US28.

| | Percentage of binding |
|---|---|
| Condition | to CHO cells |
| 1D3-mFc (1:4) 250 μg/ml | 0.66% |
| VUN100 Mv + Bv Ab mixture 50 μg/ml | 2.47% |
| 13-1C10-1B9 (1:5) (200 μg/ml) | 0.71% |
| 13-1C10-1C10 (1:5) (200 μg/ml) | 0.62% |
| 13-1C10-1G9 (1:5) (200 μg/ml) | 0.44% |
| 14-1H3-1A10 (1:5) (200 μg/ml) | 0.58% |
| 14-1H3-1C10 (1:5) (200 μg/ml) | 0.31% |
| 14-2C2-1G4 (1:20) (50 μg/ml) | 0.35% |
| 14-4E4-1A1 (1:5) (200 μg/ml) | 1.63% |
| 14-4E4-1E8 (1:5) (200 μg/ml) | 0.03% |

The data in Table 7A and FIG. 22A show that the VUN100 molecules have a consistently higher level of background binding to US28-negative CHO cells, compared to antibodies generated against the strain agnostic epitopic sequence-1 (“SAES1”) within ECD3. Note that VUN100 was tested at a higher dilution/lower concentration than most other antibodies which should assist in reducing the detected off-target binding of the VUN100 sample (since higher antibody concentrations tend to lead to higher background binding). Despite this, the data shows that the ECD3-binding molecules of the present invention consistently showed reduced background binding compared to the VUN100 sample.

Typically, the VUN100 molecules bind off-target cells by an increased level of at least 3- or 4-fold or more.

Comparatively, the further ECD3-binding molecules, as with the 1D3 binding molecule previously described, demonstrate much lower background binding to US28-negative CHO cells.

Accordingly, the ECD3-binding molecules of the present invention, generated against the strain agnostic epitopic sequence-1 (“SAES1”) within ECD3, consistently demonstrate a more therapeutically-relevant activity with a lower risk of off-target binding compared with VUN100 molecules.

A further comparison has been made between the binding molecules of the present invention (such as 4H3C3, 7B1F3 and 2F5B11) which bind to a conserved strain agnostic epitopic sequence-2 (“SAES2”) that has been identified by the inventor to be present within the region defined by positions 26 to 37 in ECD1 of US28, and the VUN100 Ab of WO 2019/151865 and De Groof et al, 2019 (supra) and/or the VUN100b bivalent molecule of De Groof et al, 2020 (supra), which have been reported to bind to a discontinuous epitope present within the US28 N-terminus and ECD4 of US28, wherein binding within the N-terminal region occurs within positions 1 to 22, including at least position 16, and most likely also one or more of positions 8, 18 and/or 19, and potentially other amino acids in positions 1-10 and 16-22 (see the discussion in Background section of this application for further details of the characterisation of the discontinuous epitope bound by the VUN100 molecules). An exemplary ECD3-binding molecule of the present invention, 13-5G6-1D3, was used as a positive control following the previous comparative data with VUN100 binding molecules.

Table 7B and FIG. 22B summarises the percentage of binding for ECD1-binding molecules in flow cytometry in control CHO cells (i.e. not expressing US28), which is indicative of the risk for off-target binding, with the background levels for the secondary antibodies removed. Therefore, the percentage of binding indicated by each condition is off-target binding of the tested binding molecules.

TABLE 7B
Percentage of binding for ECD1-binding molecules
to CHO cells that are negative for US28.

| | Percentage of binding |
|---|---|
| Condition | to CHO cells |
| 1D3-mFc (1:4) 50 μg/ml | 0.57% |
| VUN100 Mv + Bv Ab mixture 50 μg/ml | 2.87% |
| 2F5B11 (1:5) | 0.39% |
| 2G2B3 (1:5) | 0.95% |
| 4A11A11 (1:5) | 0.76% |
| 4H3C3 (1:5) | 0.31% |
| 5D6H11 (1:5) | 0.64% |
| 5E1E4 (1:5) | 1.64% |
| SE8F7 (1:5) | 0.96% |
| 6G3D6 (1:5) | 1.24% |
| 7B1F3 (1:5) | 0.67% |
| 9G3B9 (1:5) | 1.17% |

The data in Table 7B and FIG. 22B show that ECD1-binding molecules can be generated against the strain agnostic epitopic sequence-2 (“SAES2”) within ECD1 with decreased levels of background binding compared with VUN100. ECD1-binding molecules of the present invention, in all cases, showed reduced background binding compared to the VUN100 sample.

Accordingly, the data show that ECD1-binding molecules of the present invention, generated against the inventor's identified strain agnostic epitopic sequence-2 (“SAES2”) within ECD1, can be reliably developed that have a more therapeutically-relevant activity with a lower risk of off-target binding compared with VUN100 molecules.

While either improved strain agnostic binding and/or low background levels are sufficiently advantageous properties for the ECD3- and ECD1-binding molecules, compared to the VUN100-based molecules, the applicant further investigated whether ECD3- and ECD1-binding molecules could be readily obtained that have a binding specificity that improves on that of VUN100 molecules. Therefore, five exemplary ECD3-binding molecules and seven exemplary ECD1-binding molecules, were compared with the already exemplified promising 1D3 candidate, and VUN100 as a control. The five exemplary ECD3- and ECD1-binding molecules are as listed in Tables 8A and 8B, respectively.

In order to calculate binding specificity for US28-expressing CHO cells, compared to control cells, the level of specificity was calculated for exemplary ECD3- and ECD1-binding molecules as follows: background levels were subtracted as described above, the CHO-US28 binding level for each test antibody sample was divided by its respective control CHO binding level (i.e. a ratio of positive binding to negative binding), and the difference (relative to the binding specify determined for the VUN100 sample) was calculated, wherein a value >1 represents an improved specificity compared to VUN100.

The ECD3 results of these experiments are represented in Table 8A and FIG. 23. The ECD1 results of these experiments are represented in Table 8B and FIG. 24.

TABLE 8A
Binding specificity for 5 exemplary ECD3-binding subclones.

| | Binding specificity comparative to the |
|---|---|
| Condition | specificity displayed by VUN100 |
| VUN100 Mv + Bv Ab mixture | 1.00 |
| 14-202-1G4 (1:20) | 5.37 |
| 14-484-1E8 (1:5) | 4.60 |
| 14-1H3-1A10 (1:5) | 2.29 |
| 13-1C10-1C10 (1:5) | 1.74 |
| 1D3-mFc (1:4) 50 μg/ml | 1.65 |

TABLE 8B
Binding specificity for 7 exemplary ECD1-binding subclones.

| | Binding specificity comparative to the |
|---|---|
| Condition | specificity displayed by VUN100 |
| VUN100 Mv + Bv Ab mixture | 1.00 |
| 1D3-mFc (1:4) 50 μg/ml | 1.65 |
| 2F5B11 (1:5) | 4.21 |
| 2G2B3 (1:5) | 1.24 |
| 4A11A11 (1:5) | 3.20 |
| 4H3C3 (1:5) | 3.35 |
| 5D6H11 (1:5) | 1.18 |
| 7B1F3 (1:5) | 1.67 |
| 9G3B9 (1:5) | 1.10 |

The binding specificity data demonstrate that a number of additional ECD3 subclones provided, and even increased, the improved specificity for the 1D3-Fc binding molecule that was raised against ECD3, and also demonstrated improved binding specificity over the VUN100 molecules.

This demonstrates that multiple ECD3-binding molecules, generated against the strain agnostic epitopic sequence-1 (“SAES1”) within ECD3, can readily be identified in accordance with the protocols described herein that have improved binding specificity compared to 1D3, and/or which at least have improved binding specificity compared with VUN100 molecules.

Overall, these data demonstrate that binding molecules with ECD3-binding specificity, as produced in accordance with the protocols described herein against the strain agnostic epitopic sequence-1 (“SAES1”) within ECD3, can be provided to exhibit one, two or all three of the following characteristics:
1. a consistently improved strain agnostic binding to US28 encoded by different HCMV strains, compared to VUN100 molecules;
2. a consistently substantially lower background binding to US28-negative cells compared with VUN100 molecules; and optionally
3, maintained or preferably improved binding specificity compared with VUN100 and/or 1D3 molecules.

Similarly, these data demonstrate that a number of ECD1-binding molecules, generated against the strain agnostic epitopic sequence-2 (“SAES2”) within ECD1, can readily be identified in accordance with the protocols described herein that have improved binding specificity compared with VUN100 molecules, and even some that have improved binding specificity compared to the exemplary anti-ECD3 antibody of the present invention, 1D3.

### Example 3

### Binding on HCMV Infected Human Tissues

HCMV infected human lung tissue was used to validate an exemplary ECD3-binding molecule of the present invention, US28-13-5G6-1D3 rAb, binding on HCMV infected human tissues by using immunohistochemistry analysis (IHC).

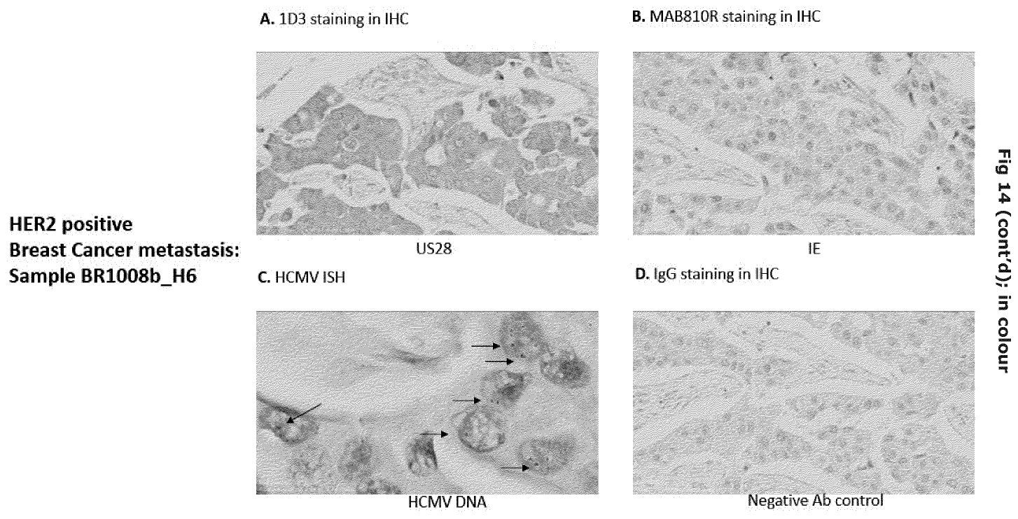

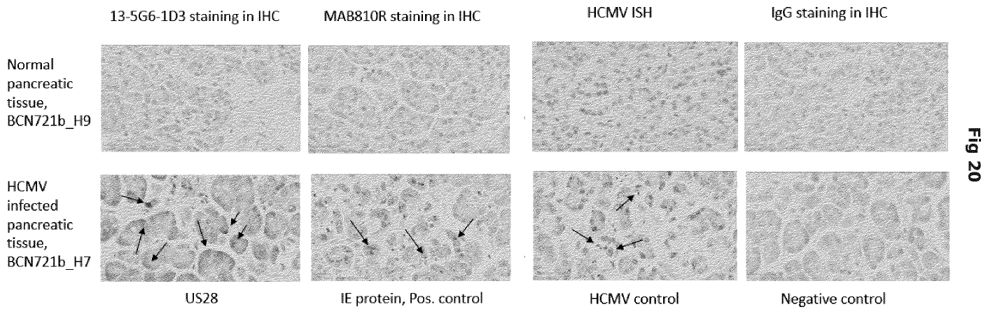

The US28-13-5G6-1D3 rAb showed staining between 1:400-1:1600 dilution (w/v), and 1:400 dilution (w/v) was selected for the further screening studies (FIG. 12 A). The HCMV anti-IE commercial antibody MAB810R was used as positive control and showed optimal staining for HCMV infected lung tissue in 1:200 (w/v) dilution (FIG. 12 D) as recommended in the manufacturer's manual. IgG was used as negative control antibody (FIG. 12 F). The staining of the HCMV infected lung tissue with the US28-13-5G6-1D3 rAb demonstrated specificity for HCMV infected alveolar cells as there were no staining of the alveolar cells and fibroblasts in the normal lung tissue (FIG. 12 B) or of HCMV negative myometrium (data not shown). The US28-13-5G6-1D3 rAb stained typical HCMV infected cells with the characteristic cytomegalo effect in the HCMV positive control slide (FIG. 12 A). The 13-5G6-1D3 rAb showed strong cytoplasmic staining in these cells as the US28 protein is known to be located in cytoplasm and cell membrane, whereas the anti-IE control antibody MAB810R showed nuclear staining, which is as expected since the IE proteins are known to be located in cell nucleus during the productive HCMV infection (FIG. 12 D). The positive staining was verified to be HCMV specific by using in situ hybridization (ISH) method with a probe targeting HCMV DNA in the same samples. Interestingly, in the normal lung tissue, the 13-5G6-1D3 rAb stained positively for some tissue macrophages (FIG. 12 B), which are known to be carriers of latent or reactivated HCMV. The same macrophages showed presence of HCMV DNA as demonstrated by positive ISH signals (FIG. 12 C). The same macrophages did not show positive staining for the IE protein (FIG. 12 E). The staining with the negative antibody control anti-IgG Ab remained negative for the lung tissue (FIG. 12 F). These results are consistent with our other results presented herein from the cell studies showing specific binding of the US28-13-5G6-1D3 rAb to HCMV infected human tissue. The FIG. 20. Shows specific staining results of additional example of productive HCMV infection in morphologically normal, adjacent tumor (NAT) pancreatic tissues.

Following the positive data with 1D3, HCMV infected human lung tissue was used to validate three exemplary ECD1-binding molecules of the present invention, US28-4-4F3C3, US28-4-7B1F3 and US28-4-2F5B11, binding on HCMV infected human tissues by using immunohistochemistry analysis (IHC).

US28-4-4F3C3 rAb (1:800 dilution (w/v)), US28-4-7B1F3 rAb (1:6400 dilution (w/v)) and US28-4-2F5B11 rAb (1:1600 dilution (w/v)) all showed positive staining (FIG. 25 B-D). The previously exemplified US28-13-5G6-1D3 rAb was used as positive control and showed optimal staining in these studies for HCMV infected lung tissue in 1:3200 (w/v) dilution (FIG. 25 A). IgG was used as negative control antibody. The staining of the HCMV infected lung tissue with the exemplified ECD1-binding molecules demonstrated specificity for HCMV infected alveolar cells as there were no staining of the myometrial cells and fibroblasts in the corresponding HCMV negative myometrium controls. As with the US28-13-5G6-1D3 rAb, the ECD1-binding molecules stained typical HCMV infected cells with the characteristic cytomegalo effect, showing strong cytoplasmic or membranous staining in these cells as the US28 protein is known to be located in cytoplasm and cell membrane, and stained positively for some tissue macrophages, which are known to be carriers of latent or reactivated HCMV.

### BINDING on HCMV Infected Human Cancers

We thereafter studied the binding of US28-13-5G6-1D3 rAb to human cancer tissues by using IHC and confirmatory ISH analyses. The results were evaluated by a Senior Pathologist. We found specific staining of the US28-13-5G6-1D3 rAb on several cancer types. Among these were glioblastomas (e.g. glioblastoma grade 4), colonic cancer, rectum cancer, breast cancer (e.g. locally advanced breast cancer and its metastasis), oesophagus cancer, gastric/stomach cancer, pancreas cancer, liver cancer such as hepatocellular carcinoma, lung cancer, malignant pheochromocytoma and cervical cancer, showing for the first time a potential universal role of US28 in pathology of cancer. The staining intensity of different cancer cells were classified as negative=0, weakly positive=1+, moderately positive=2+ or strongly positive=3+. The US28 staining was only classified positive in case the tumor cells had positive staining that was distinctive from the adjacent normal tissues. In the majority of tumors with a positive US28-13-5G6-1D3 rAb staining, the staining was generally positive in large parts of the tumor cells. The tumor positivity was confirmed by positive finding of HCMV DNA in nucleus of the tumor cells by using ISH. On many occasions the tumor samples showed positive staining for vessel endothelial and smooth muscle cells, which were also seen in adjacent normal tissues and which was not considered as sign for tumor positivity. The IgG control Ab staining was used as negative control and was negative in all of the studied cancerous and normal tissues.

There was positive US28-13-5G6-1D3 rAb staining intensity combined with finding of nuclear HCMV DNA in one of the three studied oesophagus and stomach adenocarcinoma samples. The HCMV positive stomach tumor showed morphological signs of diffuse type of gastric cancer, which normally grows in small cell groups and affects large areas, rather than just one area of the stomach. The positive tumor sample was from a 56 years old male, whose tumor was grade 3 and TNM-classified as T3N1M0. Also, the US28 positive oesophagus cancer showed aggressive type with stage IIIa, T3N1M0. All (3/3) of the adjacent oesophageal tissues were negative for the epithelial cell staining and 2/3 stomach tumor adjacent samples were also negative for HCMV DNA.

The staining intensity with the US28-13-5G6-1D3 rAb was positive (1+) in addition to positive HCMV DNA analysis in 1/3 of the tested colon adenocarcinomas, whereas 3/3 controls were all negative for the US28-13-5G6-1D3 staining in adjacent epithelial cells. HCMV DNA could still be traced in 2/3 adjacent samples. The positive tumor was of grade 3 with TNM classification of T4N1M0. Of note, the T4 staging means that the cancer has penetrated through the wall of colon and may have attached or grown into other nearby tissues or organs, and is therefore classified as a locally advanced colon cancer by definition.

All 3 studied rectum adenocarcinoma samples had positive US28-13-5G6-1D3 staining and positive HCMV ISH. They were T3N0M0, grade 2; T4N0M0, grade 2; and T3N1M0, grade 3. Of the adjacent samples 2/3 were negative for HCMV and 1/3 had both positive US28-13-5G6-1D3 staining and positive ISH.

2/3 of the studied Hepatocellular carcinomas were positive for US28-13-5G6-1D3 staining and HCMV ISH. Strikingly, the adjacent liver tissues showed signs for productive HCMV infections with moderate US28-13-5G6-1D3 staining and large and frequent cytoplasmic HCMV DNA aggregates in Kupffer cells. This was both seen in the tumor adjacent hepatic tissue and the separate adjacent samples.

We also found both US28-13-5G6-1D3 staining and positive ISH in 2/3 lung cancer samples, 2/3 cervix cancer samples and 1/3 pancreas cancer samples.

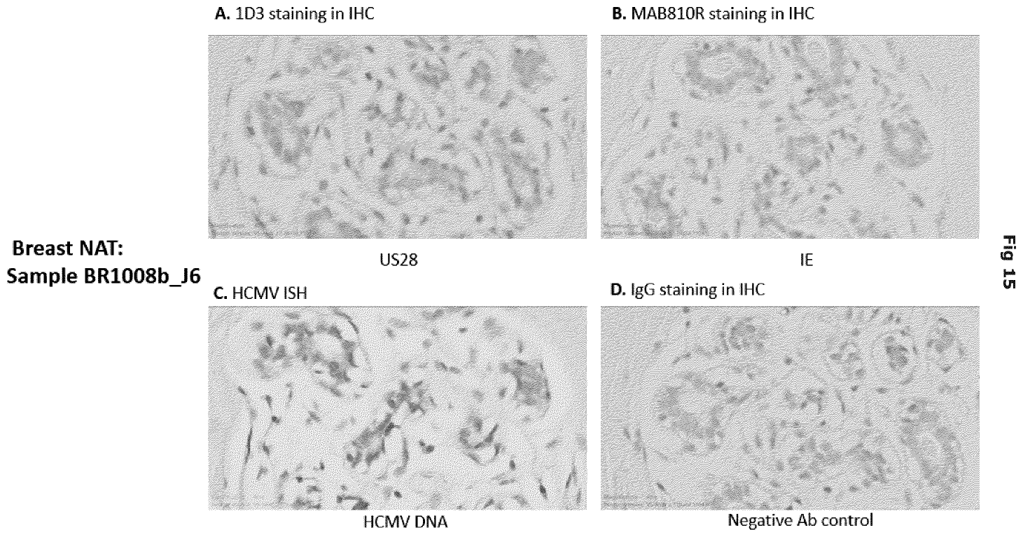

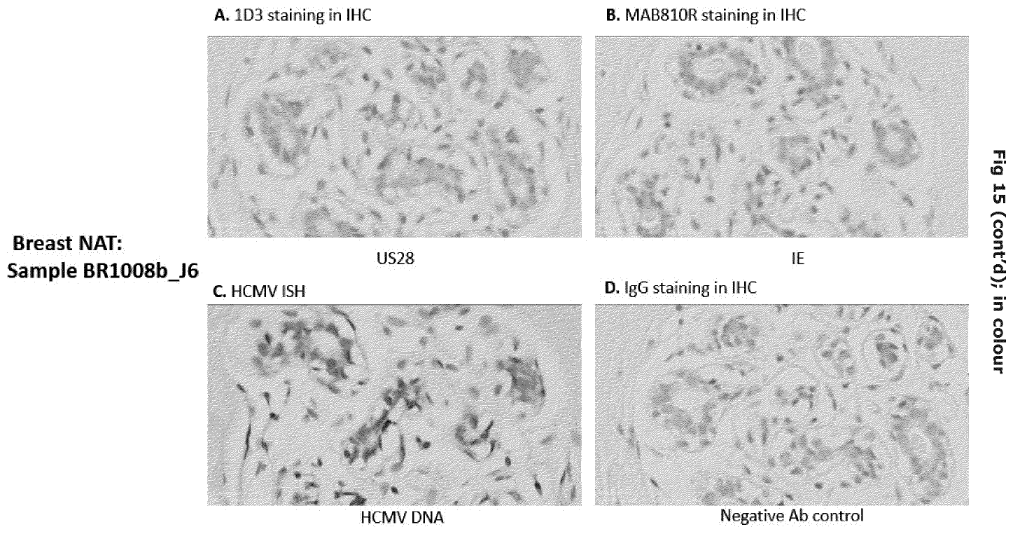

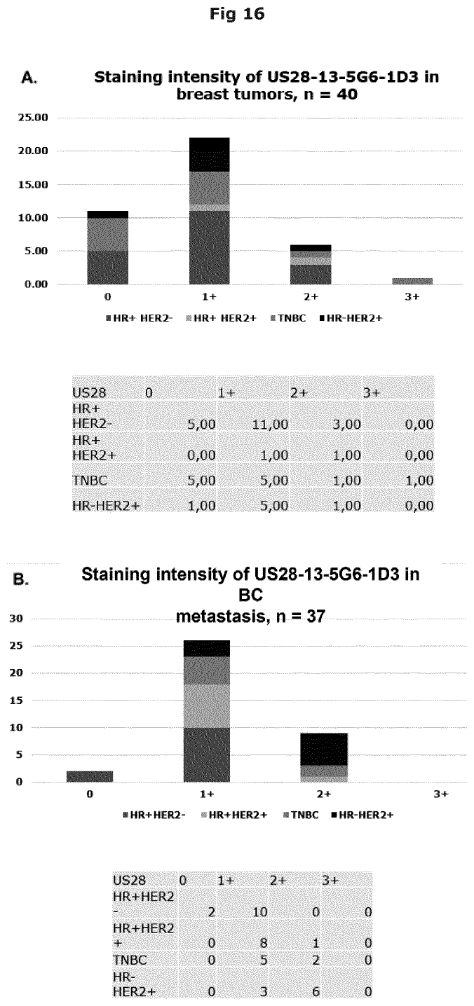

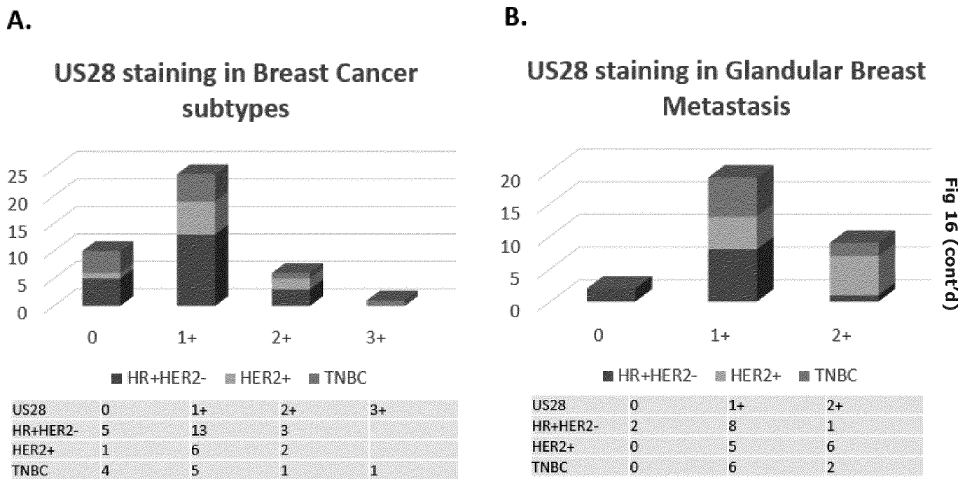

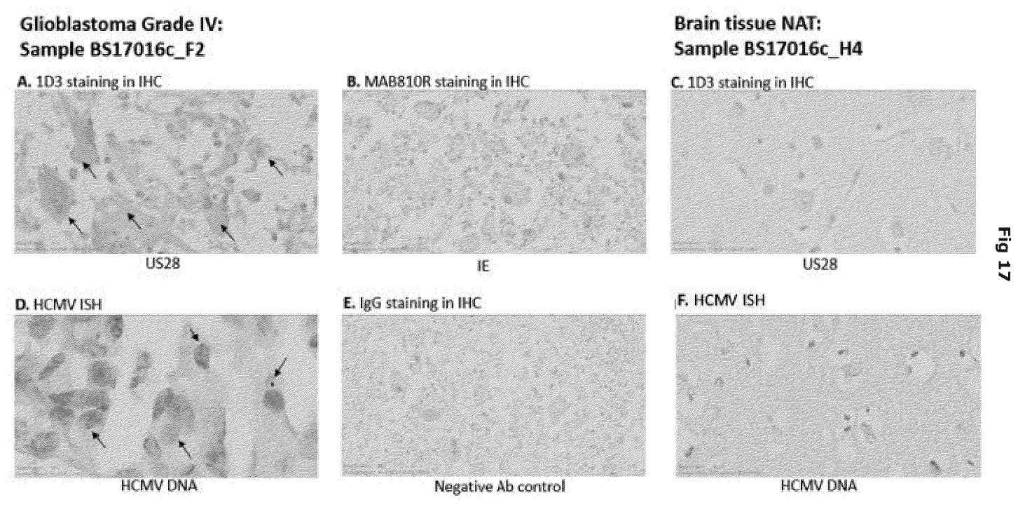

We then studied the HCMV US28 expression in a breast cancer cohort in more detail by using IHC and ISH. ˜74% of the 41 studied primary breast tumor samples showed positive and specific cytoplasmic staining of US28-13-5G6-1D3 rAb and positive nuclear HCMV DNA in the cancer cells (FIGS. 13 and 15). Surprisingly, the more aggressive the tumor forms, such as those that either had low expression of hormone receptors, were HER2 positive or triple negative breast cancer (TNBC), a tendency for stronger staining was seen in tumor cells for the US28-13-5G6-1D3 rAb (FIGS. 13 and 16 A). The US28-13-5G6-1D3 rAb staining was limited to tumor cells, although also many tumor vessels showed strong, positive staining. Interestingly, ˜94% of the studied 30 metastatic lymph nodes showed specific, US28-13-5G6-1D3 rAb staining for the metastatic cells. The most intensive staining was seen for TNBC and HER2 positive metastases (FIGS. 14 and 16 B). All TNBC (n=8) and HER2 positive (n=11) metastases had positive US28-13-5G6-1D3 staining (FIG. 16 B). The HCMV ISH was positive for 98% of the studied breast cancer samples (n=41) and 100% of the studied glandular breast cancer metastasis (n=30). Interestingly, the only HCMV DNA negative breast cancer sample was a neuroendocrine tumor, which also showed negative US28-13-5G6-1D3 staining. The ISH findings were also consistent with the above listed IHC results showing many nuclear HCMV DNA signals per tumor cell in only 12% of the primary tumors, whereas there were many nuclear ISH signals per tumor cell in 39% of the metastasis. The many ISH signals per tumor cell were in both primary tumors and metastasis seen exclusively, with only one or two exceptions, in triple negative and HER2 positive tumors. There was no cytoplasmic staining of U28-13-5G6-1D3 in normal breast epithelial cells and 10/10 adjacent control breast tissues (FIG. 15). The normal lymphoid tissue did not either show any positive US28 staining (2/2 samples were both negative). However, many of the adjacent tissues showed positive staining for the vessel endothelial and smooth muscle cells (in 70% of the adjacent breast tissues), which was similar to the tumor samples. Consistently, HCMV DNA was also found in small amounts in 3/10 normal, adjacent breast tissues. The 13-5G6-1D3 staining was evaluated to be specific for the HCMV infected cells in both the breast cancer and adjacent normal tissues.

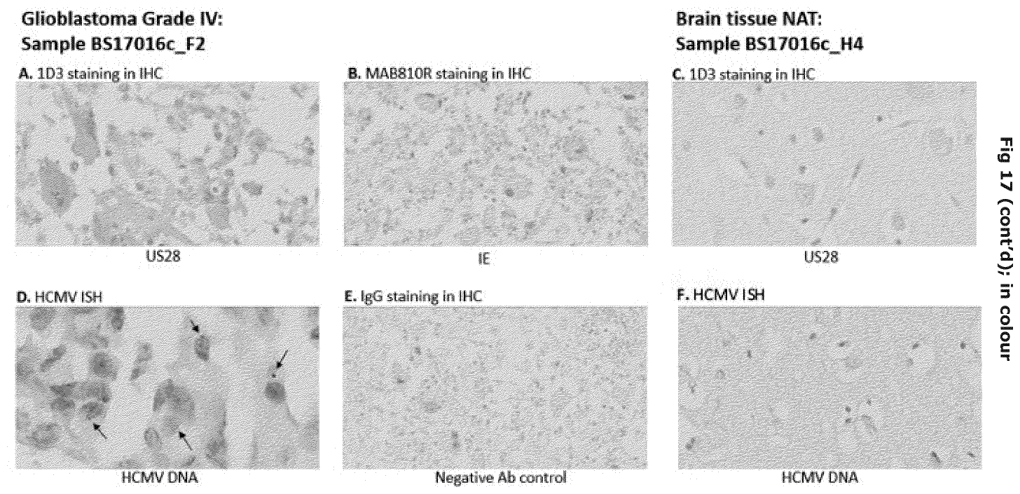

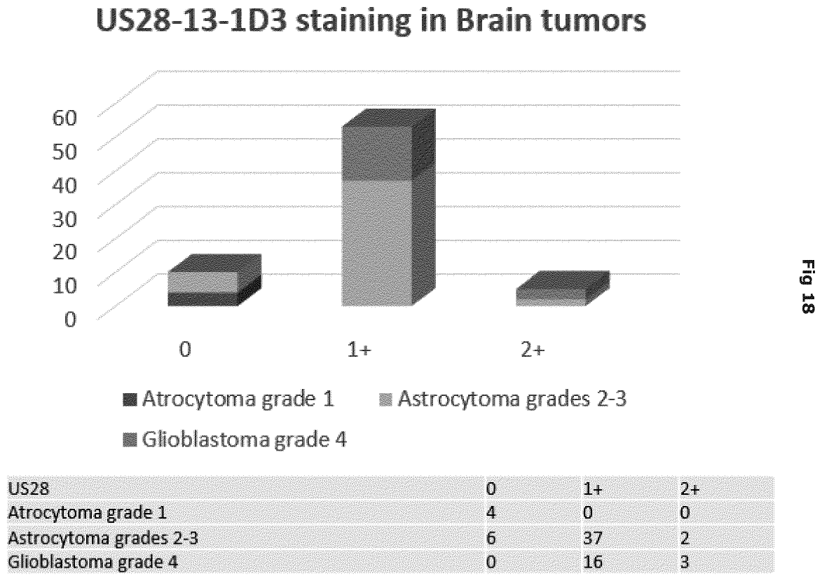

Several earlier studies have documented HCMV infection and US28 expression in glioblastoma tissues [5, 6]. We stained human astrocytoma grade 1-3 and glioblastoma grade 4 tissues with the US28-13-5G6-1D3 rAb and MAB810R antibody. All the astrocytoma grade 1 (n=4) were negative for the US28-13-5G6-1D3 rAb staining. Of the grade 2-3 astrocytomas, 6 were negative, 37 were positive (1+), and 2 were moderately positive (2-F) for the US28-13-5G6-1D3 rAb staining. All of the glioblastoma grade 4 (n=19) were positive, of which 3 were moderately positive (2-F) for the US28-13-5G6-1D3 rAb staining (FIGS. 17 and 18). The anti-IE antibody MAB810R showed positive staining for 17 of 19 glioblastoma grade 4 samples, whereas 2 samples were considered negative. The HCMV ISH was positive in all tumor samples indicating presence of HCMV DNA in 100% of the samples. The adjacent, normal brain tissue controls did not show any general staining for the US28-13-5G6-1D3 rAb (FIG. 17 C). Five NAT were completely negative for HCMV DNA. The HCMV DNA was found in astrocyte and glia cell nucleus in the resting five NAT samples. The negative IgG control staining in tumor tissues was negative (FIG. 17 C) indicating the HCMV US28 specific staining by 13-5G6-1D3 rAb in brain tissues.

Only 6/41 studied breast cancer samples, but most glioblastoma grade 4 samples (17/19) showed positive cytoplasmic staining for the anti-IE MAB810R antibody. The positive breast cancer samples often contained only rare positive cells with positive cytoplastic staining, but 5/6 of the positive samples were either HER2 positive (n=4) or TNBC (n 1). In addition, one HER2 positive sample stained positively for MAB810R Ab in tumor macrophages. Only 1/37 metastases stained positively for the MAB810R Ab, but this sample was also HER2 positive. The staining of the MAB810R Ab was always combined with a positive staining with the US28-13-5G6-1D3 rAb in breast cancer samples. Very interestingly, several tumor samples with negative MAB810R staining showed positive vascular endothelial and smooth muscle cell staining, which was positive for both the US28-13-5G6-ID3 rAb and MAB810R antibody.

The cytoplasmic staining of the HCMV IE proteins has been previously described in several cancer types [7, 8]. The MAB810R monoclonal antibody was used as HCMV positive control in our experiments to target the intra-cellular, HCMV encoded IE1 and IE2 proteins. These proteins share the exons 1-3 and include additionally either exon 4 (IE1) or 5 (IE2). The full-length IE proteins are required for the HCMV replication and reactivation from latency to cause productive HCMV infection [4]. They are not expressed during HCMV experimental latency [9]. However, RNA and protein expression of exon 4 of the IE1 protein (IE14) (˜60 kDa of size) has previously been shown in latently HCMV infected CD34+ hematopoietic precursor cells but not in CD14+ monocytes in both natural and experimental latency systems [10]. Thus, the HCMV gene expression during latency in addition to US28 protein may include expression of IE1×4 but not the full-length IE1 and 2 proteins [10]. According to the information from the manufacturer (MerckMillipore.com), the MAB810R antibody binds on the ˜68-72 kDa full-length IE1 and IE2 proteins indicating that the MAB810R Ab targets cells with a lytic or replicative CMV infection. It should therefore not bind to latently HCMV infected cells. Our results show that the majority of cancer cells in the positive breast tumor samples expressed the US28 protein, but not the IE proteins.

In our studies 100% of the breast cancer metastases and 100% of the brain tumors were all HCMV DNA positive when studied with ISH. We observed that in malignant tumors, the HCMV DNA was exclusively located in the tumor cell nucleus. The number of nuclear HCMV DNA signals vary between different tumor samples. We also observed that the number of nuclear ISH signals per tumor cell was increased in aggressive cancer types such as TNBC and HER2 positive breast cancers. The nuclear localisation of the ISH signals was not observed in normal tissues where the viral DNA was mainly located in the cytoplasm, in multiple large aggregates (FIG. 20). During productive HCMV infections, the virus particles are packed in the endoplasmic reticulum before they are excreted from the cells. This explains the cytoplasmic location of most of the DNA during the productive HCMV infection. We observed such productive HCMV infections in normal tissues only (and NAT) such as pancreatic (FIG. 20), breast, liver and lung tissues (FIG. 12).

When taken together, these data demonstrate that the cancer cells, and especially breast cancer cells, carry a latent HCMV infection. This finding is supported in our studies not only by the lack of IE protein expression and the lack of cytoplasmic viral aggregates, but also by the presence of nuclear HCMV signals in the tumor cells. Our results and conclusion are supported by earlier data indicating the lack of cytopathic effect and lack of evidence of replicating virus in tumor cells [11, 12].

The latently HCMV infected monocytes are programmed for prolonged cell survival [13] and reasonably, the latent HCMV infection in tumor cells may contribute to increased tumor cell survival. The latently HCMV infected monocytes are shown to exert wide immunosuppressive functions in their microenvironment to protect themselves from elimination by the immune system [13]. This is also similar to cancer cells, and evading immune destruction is acknowledged as one of ten hall marks of cancer [14].

Interestingly, as also described by others earlier [7, 8], we found cytoplasmic full-length IE protein expression in a few aggressive breast cancer samples and most of the glioblastoma 4 samples. The cancer specific epigenic mechanisms, such as histone modifications, DNA hypomethylation, or certain viral or host mutations, may inhibit viral replication, but still allow some IE expression in certain aggressive cancers [15]. Our finding of the presence of IE protein expression in the cytoplasm of only a few cells of some positive breast tumor samples, indicates that the heterogenous cancer cell populations may contain few cells/cell types that are more permissive for a replicative virus. It has been proposed that the cancer stem cells are especially permissive for the HCMV infection and that the IE protein expression in glioblastoma cells is mutagenic and induces cell survival, tumor growth and epithelial-mesenchymal (EMT) phenotype [16]. The latently HCMV infected, US28 positive cancer cells, may further create a strong immunosuppressive microenvironment, which could allow the expression of IE proteins, which are known to be highly immunogenic CD4+ and CD8+ T cell antigens [17], in few tumor cells. Our results show, additionally, that the tumors contain other HCMV infected cells, which stain positively for the IE proteins, such as endothelial and smooth muscle cells and sometimes macrophages. These cell types are known to be more permissive for the replicative virus infection than the tumor cells [16], and might additionally contribute to the viral maintenance and shedding within the tumor. However, the mechanisms involved in HCMV related infection in glioblastoma tumors might be different from breast cancer due to different cellular origin of these cancers.

Taken together the above listed results, we show here that binding agents which bind specifically to ECD3 of US28, as exemplified herein by the US28-13-5G6-1D3 rAb, bind specifically on the surface of HCMV infected cells as shown by the binding of US28-13-5G6-1D3 rAb on the US28 overexpressing cell model, HCMV infected cells and tissues, PBMCs from HCMV seropositive individuals and HCMV positive cancer cells in tumor tissues. In contrast, binding agents which bind specifically to ECD3 of US28, as exemplified herein by US28-13-5G6-1D3 rAb, do not generally bind on HCMV negative control cells or tissues (as shown in FIGS. 12, 15, 17, 19 and 20). These findings are coherent with the role of US28 protein, which is shown to be present during both the lytic and latent HCMV infections [18]. We show here for the first time positive US28 staining in human breast, rectum, oesophagus, stomach, pancreas, liver, lung and cervical cancers and confirm some earlier results of HCMV US28 expression in glioblastoma grade 4 [5, 6] and colon cancer [19]. Most importantly, we show specific binding of the US28-13-5G6-1D3 rAb to locally advanced colon cancer, metastatic breast cancer tissues (˜94%) and glioblastoma grade IV tumors (˜100%) confirming Our hypothesis of the high HCMV US28 expression in aggressive, locally advanced and metastasising human cancers, and providing a further role for binding agents which bind specifically to ECD3 of US28.

These findings support the HCMV US28 role as an important therapeutic target against HCMV infections (with particular interest in latent HCMV infections) and against HCMV positive cancers (with particular interest in aggressive, locally advanced and metastasising, HCMV positive cancers), and point to antibodies and other binding agents against ECD3 of US28, particularly those binding agents with high specificity for US28 and/or strain agnostic binding, such as antibody US28-13-5G6-1D3, as highly potent, therapeutic, prophylactic, and diagnostic binding molecules. This includes the ability to provide highly-specific targeting for cytotoxic activities against HCMV infections (with particular interest in latent HCMV infections) and against HCMV positive cancers.

Binding on Primary Tissues not Infected with HCMV

When considering a therapeutic molecule, it is important to assess the risk of off-target binding for the binding molecules. This is particularly important for US28, which has a level of sequence homology to CCR5, which may be present in a subject. High binding specificity and low off-target binding is therefore an indicator of a promising therapeutic binding molecule with a low risk of off-target effects.

Therefore, we studied the binding of exemplary binding molecules of the present invention to a variety of human primary tissues to assess the level of off-target binding. These data were generated using US28-13-5G6-1D3 antibody, prepared in the same way as previously described, in flow cytometry (FACS).

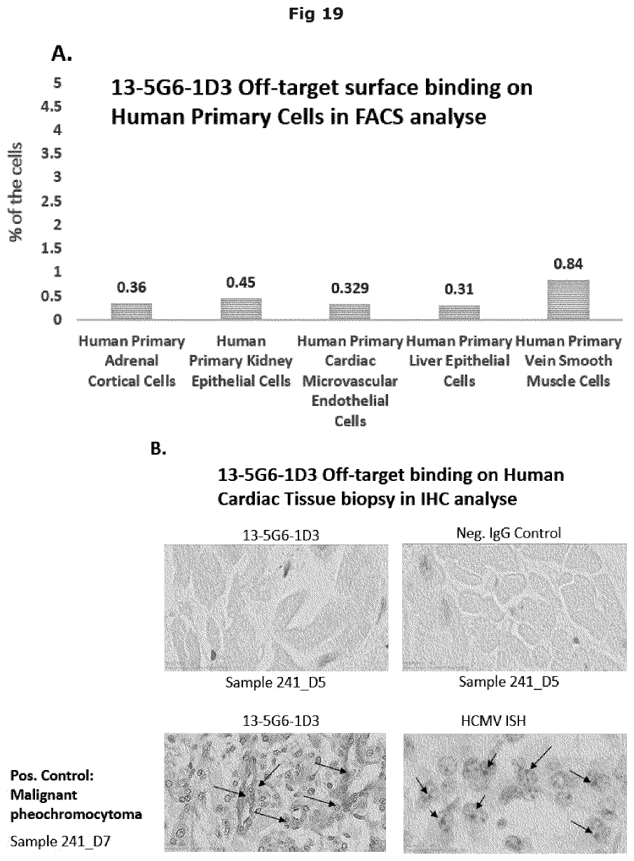

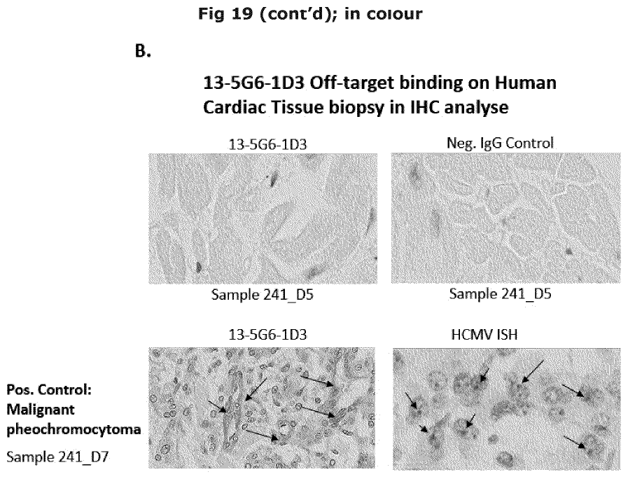

Flowcytometry analyses with the 13-5G6-1D3 antibody were conducted on various human primary cells to exclude general off-target binding. The following cell lines were studied: Human primary adrenal cortical cells, Human primary kidney epithelial cells, Human primary cardiac microvascular endothelial cells, Human primary liver epithelial cells and Human primary vein smooth muscle cells. US28 targeting antibody 13-5G6-1D3 showed very low surface binding, far below <1% on all these cell types indicating that there is no off-target binding of the 13-5G6-1D3 against these cell types (FIG. 19 A). These results support our observations on laboratory cells (i.e. non-primary cell lines) showing low binding on normal US28 negative cells. The IHC staining with the US28-13-5G6-1D3 Ab of human pancreatic tissue showed specific staining for the HCMV infected pancreatic tissue and negative staining for the HCMV uninfected pancreatic tissue (FIG. 20).

### IHC on Human Cardiac Tissues

Human cardiac tissue samples were studied for staining of 13-5G6-1D3 by immunohistochemistry. Immunohistochemistry did not show any general off-target binding of the 13-5G6-1D3 on human heart muscle tissue (FIG. 19 B). The same muscle tissue was also negative for the HCMV DNA (Data not shown). The staining with 13-5G6-103 was similar to the staining with the negative IgG antibody control. Human malignant pheochromocytoma tumor was used in the assay as positive control for HCMV infection. It was located on the same TMA plate with the heart samples and showed positive staining for the 13-5G6-1D3 antibody (FIG. 19 B). The ISH control showed also typical HCMV DNA staining in the nucleus of the tumor cells in the same biopsy confirming the presence of latent HCMV infection in this positive control (FIG. 19 B).

These data demonstrate that the antibody US28-13-5G6-1D3 binds specifically on both productively and latently HCMV infected cells and human tissues and that it does not bind on the surface of the healthy human cells and tissues. The positive and negative results have been confirmed by other, independent surrogate markers for HCMV infection, such as staining with the MAB81 antibody and HCMV DNA control by ISH analyses, and negative control staining with the human IgG antibody.

### Example 4

### Binding Specificity of ECD1-Binding Molecules

### Introduction:

Large-scale protein arrays are versatile and sensitive platforms for antibody specificity evaluation, screening of biomarkers, protein-protein, protein-small molecule, protein-DNA, protein-RNA, and protein-lipid interactions. They are invaluable for the evaluation of protein-binding or affinity reagents that are widely employed in research and clinical applications.

The HuProt™ Human Proteome Microarray provides the largest number of unique, full-length, individually purified human proteins on a single microscope slide. It provides >21,000 unique human proteins, isoform variants, and protein fragments—covering 16,794 unique genes. This includes 15,889 of the 19,613 canonical human proteins described in the Human Protein Atlas, with broad coverage across protein subclasses. This allows thousands of interactions to be profiled in a high-throughput manner. The full-length recombinant proteins are expressed in the yeast S. cerevisiae, purified, and printed on glass slides in duplicate along with a set of control proteins (GST, BSA, histones, IgG, etc.). The full list of the different proteins included in the HuProt™ Human Proteome Microarray as used in this Example is provided as “List B” in the Research Disclosures publication (ISSN 0374-4353, Questel Ireland Ltd) with database numbers 707018, 707019 and 707020, each entitled “Large scale protein arrange target protein identities” and each digitally published on 27 Jan. 2023, the contents of each of which are incorporated herein by reference in their entirety, fo the purposes of the disclosure of the proteins defined by “List B”. “List B” begins in Research Disclosures publication 707018, is continued in Research Disclosures publication 707019, and concludes Research Disclosures publications 707020, and so the three publications are to be read consecutively as a single disclosure of the “List B” proteins. The HuProt™ microarray is not restricted to a particular type of surface coating, although the default is glass coated with ultrathin nitrocellulose film for the non-covalent, yet irreversible, capture of active proteins to the surface.

### Experimental Overview:

Samples of humanised 7B1F3 and mouse 7B1F3 antibodies were analysed for binding specificities on HuProt™ human proteome arrays. A peptide with the sequence TDVLNQSKPVTL (“Peptide 3”) was added to the arrays at 0.1 mg/ml as a control.

After blocking, the arrays were probed with the antibody samples (0.1 μg/ml) at room temperature for 1 hour. Then the arrays were washed with TBST for 10 min×3 and probed with Alexa647-anti-human IgG Fc or Alexa647-anti-mouse IgG secondary antibodies under conditions optimized by CDI Labs for signal detection.

### Data Analysis:

Non-specific hits that directly bind to the secondary antibodies were eliminated from the analysis of the samples.
CDI software was used to quantify the specificity of each individual sample to specific proteins on the array based on Z Scores.
Z score is the average Z score of the duplicate spots of a given protein (each protein is printed in duplicate on a HuProt™ array). The Z score of each spot on a given array is calculated according to the algorithm below:

Z=[F635−F635(avg)]/F635(std)
F635(avg) and F635(std) are the average and standard deviation of the F635 values of all spots on the array, respectively.
S score is the difference of the Z Scores of a given protein and the one ranked next to it. If the S score of the top hit is >3, the antibody is considered to be highly specific for the top hit.
F635 is the average foreground signal intensity of 2 replicate spots of a given protein in the detection channel (635 nm).
B635 is the average background signal intensity of 2 replicate spots of a given protein in the detection channel (635 nm).

### Results:

Samples hum7B1F3 and mouse7B1F3 both recognized Peptide 3, corresponding to the sequence of SEQ ID NO: 177 as provided in ECD1 of the HCMV protein US28, as the top hit with very high specificity, see Table 9.

TABLE 9
Signal readouts for humanised 7B1F3 versus mouse 7B1F3.

| Sample | Top Hit | Z Score | S Score | Signal | Peptide 3 Signal |
|---|---|---|---|---|---|
| hum7B1F3 | Peptide 3 | 147.646 | 123.346 | 65535* | 65535 |
| mouse7B1F3 | Peptide 3 | 145.092 | 116.095 | 49972 | 49972 |

*65535 is the saturation signal intensity.

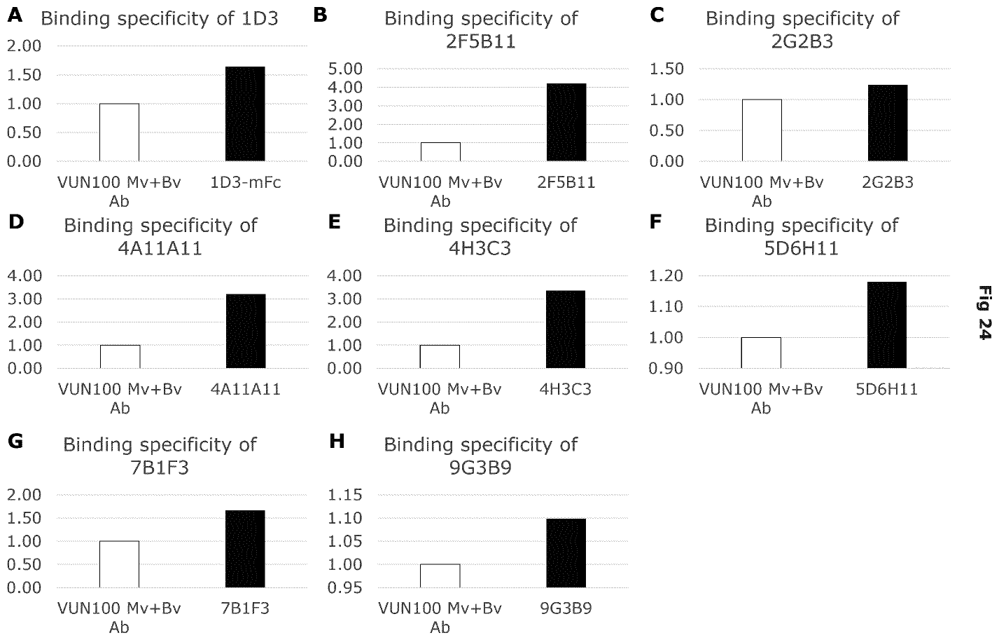

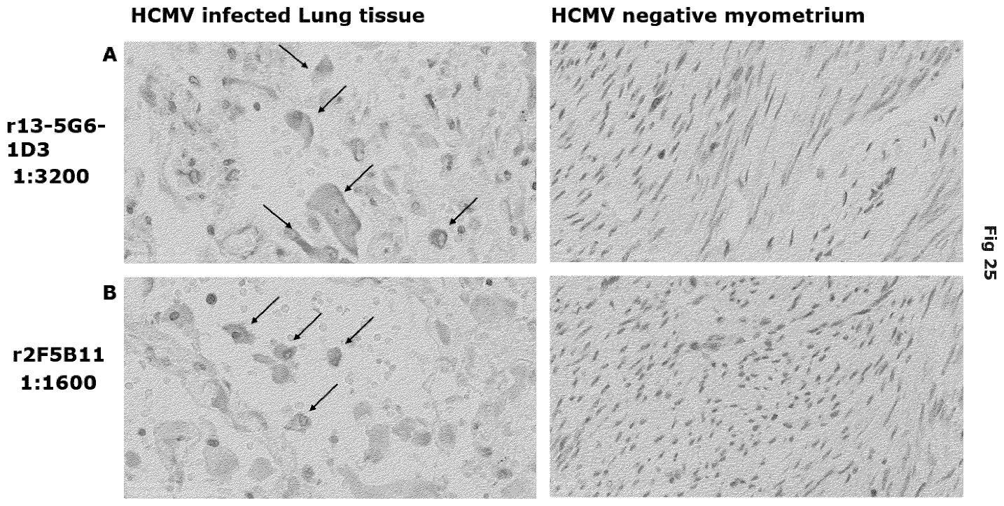

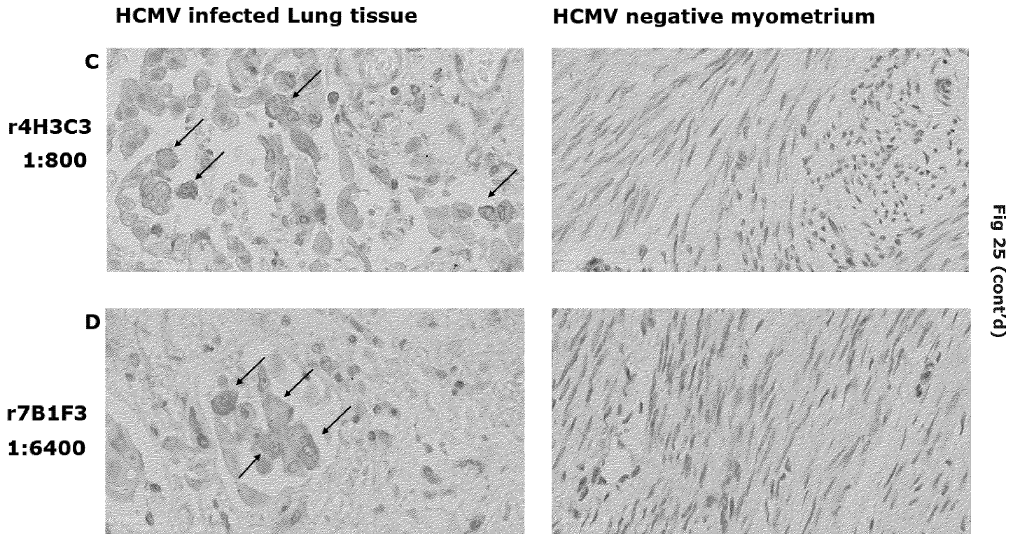

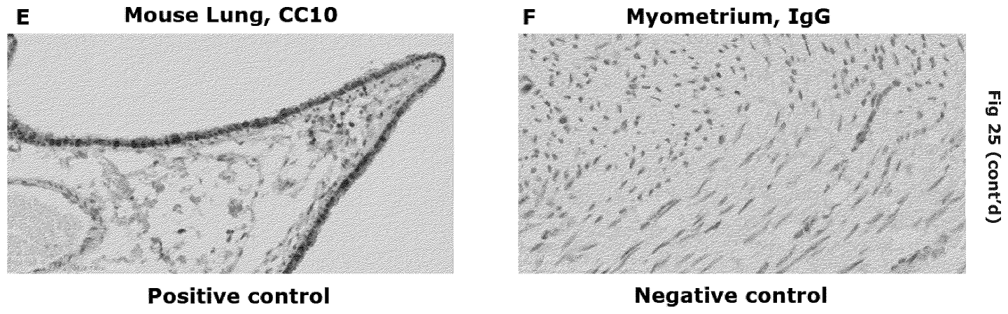

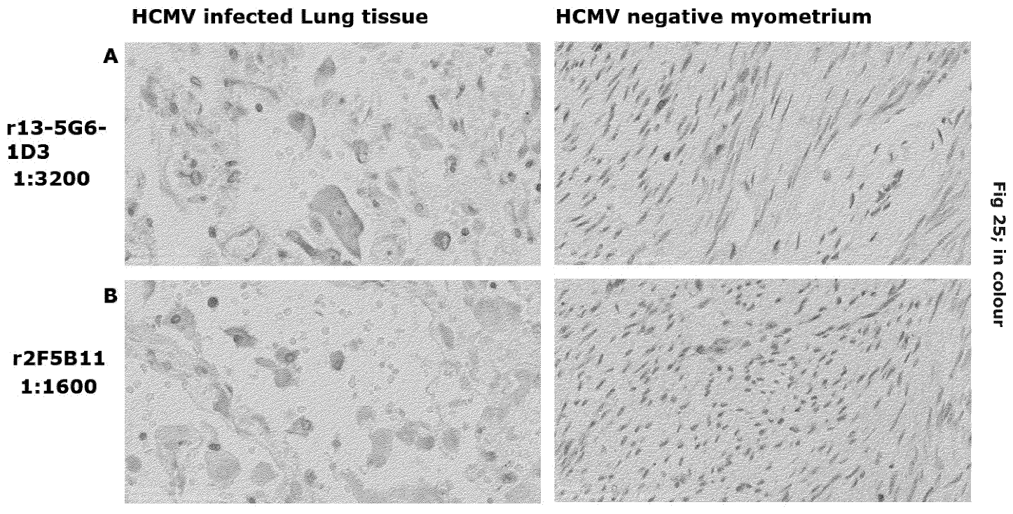

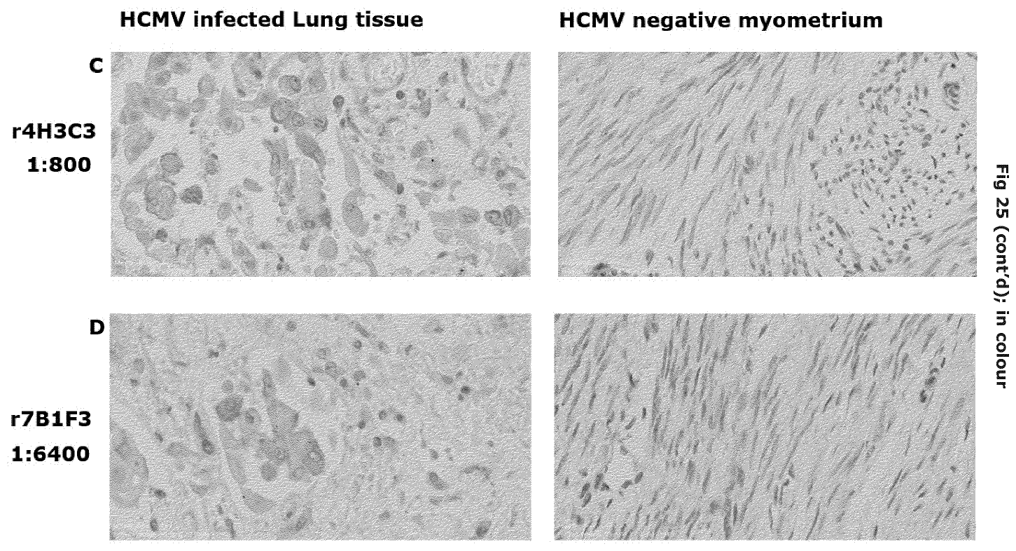

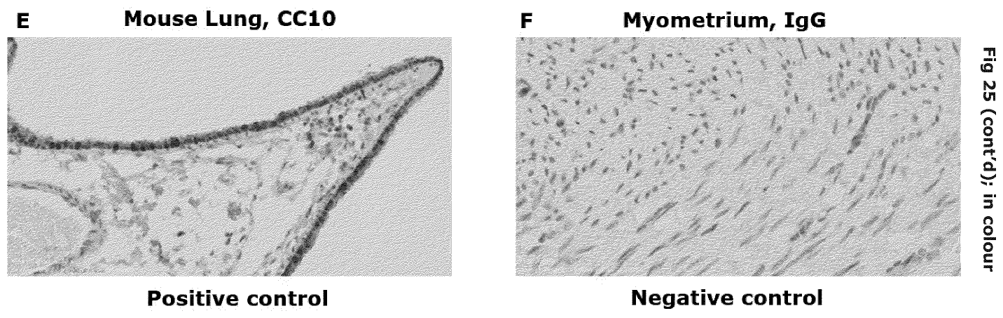

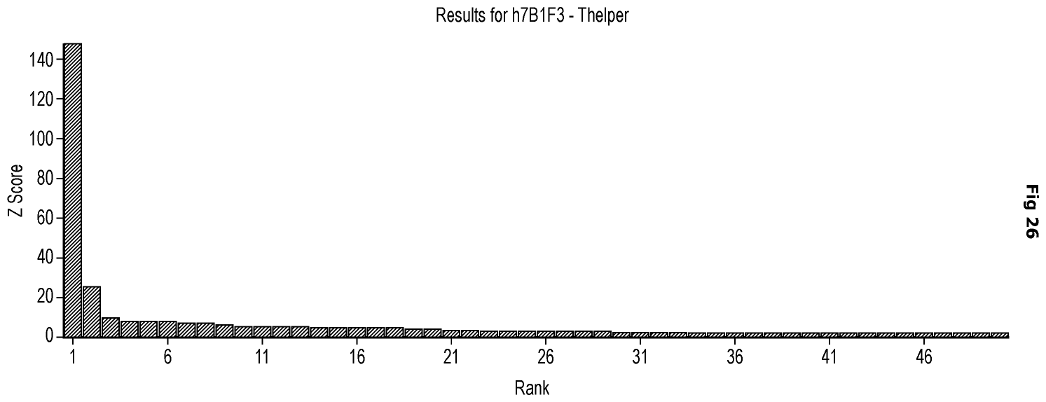

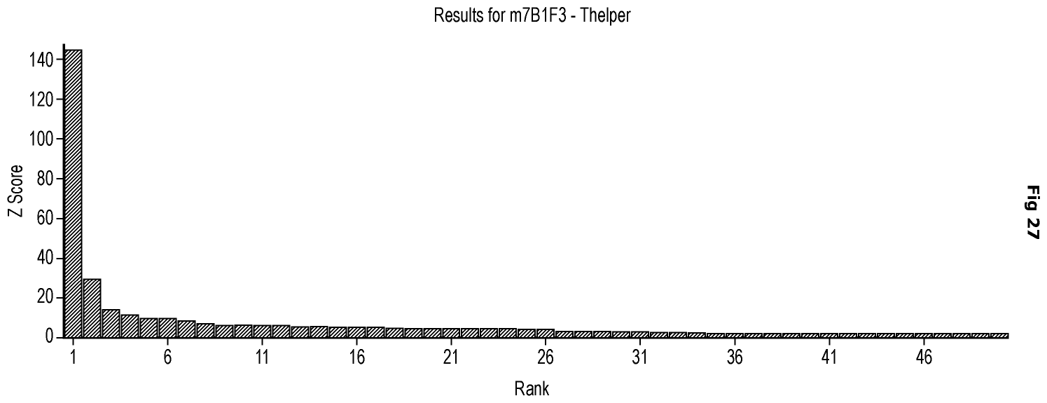

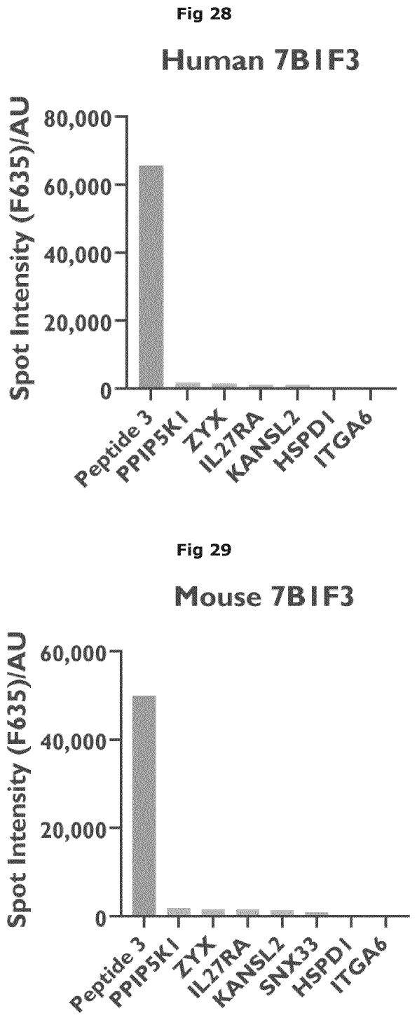

Peptide 3 was identified as the top hit with very high specificity for both hum7B1F3 (see FIG. 26 and Table 10) and mouse7B1F3 (see FIG. 27 and Table 11). In both cases, Peptide 3 was bound with very high specificity, whereas various other off-target candidates showed very low, or essentially no, binding (see FIGS. 28 and 29).

TABLE 10
Quantification of the Results for the top 10 hits from FIG. 26.

| Rank | Protein | F635 | Z Score | S Score |
|---|---|---|---|---|
| 1 | Peptide_3 | 65535 | 147.464 | 123.346 |
| 2 | AGAP2 | 10822.5 | 24.3 | 15.039 |
| 3 | EXD2 | 4151.5 | 9.261 | 1.977 |
| 4 | CCDC97 | 3274.5 | 7.284 | 0.170 |
| 5 | BYSL | 3199 | 7.114 | 0.085 |
| 6 | LASP1 | 3161.5 | 7.029 | 0.619 |
| 7 | DNAJB2 | 2887 | 6.41 | 0.100 |
| 8 | TRIM33 | 2842.5 | 6.31 | 1.300 |
| 9 | BYSL | 2266 | 5.01 | 0.012 |
| 10 | SYNCRIP | 2260.5 | 4.998 | 0.063 |

TABLE 11
Quantification of the Results for the top 10 hits from FIG. 27.

| Rank | Protein | F635 | Z Score | S Score |
|---|---|---|---|---|
| 1 | Peptide 3 | 49972 | 145.092 | 116.095 |
| 2 | AGAP2 | 10020.5 | 28.997 | 15.511 |
| 3 | FBXO2 | 4683 | 13.486 | 2.911 |
| 4 | LASP1 | 3681 | 10.575 | 1.073 |
| 5 | DNAJB2 | 3312 | 9.502 | 0.235 |
| 6 | TRIM33 | 3231 | 9.267 | 1.539 |
| 7 | EXD2 | 2701.5 | 7.728 | 1.271 |
| 8 | SYNCRIP | 2264 | 6.457 | 0.157 |
| 9 | BYSL | 2210 | 6.3 | 0.378 |
| 10 | CCDC97 | 2080 | 5.922 | 0.353 |

The top 100 hits, excluding Peptide 3, (referred to herein as “List A”) from the HuProt analysis of hum7B1F3 are as follows (with HuProt IDs in brackets): AGAP2 (JHU12842.B11C24R24); EXD2 (JHU08666.B7C25R48); CCDC97 (JHU04911.B4C30R76); BYSL (JHU15392.B11C14R64); LASP1 (JHU14849.B10C30R54); DNAJB2 (JHU05676.B1C10R90); TRIM33 (JHU19543.B15C32R26); BYSL (JHU05768.B8C20R2); SYNCRIP (JHU08142.B6C12R42); SHFM1 (JHU03919.B2C5R64); ABCF3 (JHU00673.B4C28R12); NOL4L (JHU09419.B5C16R60); PPIP5K1 (JHU07043.B5C22R20); GMPPB (JHU01291.B12C13R84); Glis2 (JHU19734.B15C19R6); SP8 (JHU16771.B13C25R40); GET4 (JHU11625.B11C17R4); ZYX (JHU08351.B8C25R40); CITED1 (JHU02424.34C9R38); IL27RA (JHU18193.B13C12R46); KANSL2_frag (JHU09033.B7C11R50); PRR16 (JHU16025.B12C28R70); KIAA0430_frag (JHU11326.B5C14R90); JO-1 Auto-antigen.B20C24R40); Glis2 (JHU19734.B15C15R8); RCAN1 (JHU11634.B10C27R4); SYNCRIP (JHU13100.B12C5R26); RRAGD (JHU11116.B8C10R80); PRKG1_frag (JHU09569.B7C10R56); PDXDC1 (JHU10332.B8C18R68); NOVA2 (JHU18112.B16C28R64); CCNY (JHU11339.B7C19R90); UBXN1 (JHU00329.B13C6R78); PALDI (JHU14658.B10C8R50); TEX33 (JHU07207.B10C14R90); POGZ (JHU12321.B9C26R14); GAS2L1 (JHU11642.B10C27R2); NOVA1 (JHU11477.B6C7R88); KJ904402 (JHU16482.B10C26R80); KANSL2_frag (JHU06156.B6C12R10); TRAF3IP2 (JHU08439.B5C13R38); PCMTD1 (JHU09086.B5C6R52); CCDC91 (JHU05488.B3C22R86); TEX33 (JHU20055.B17C9R4); ZMYM3 (JHU00766.B1C18R12); BC018766_frag (JHU01404.B3C4R20); CNKSRI (JHU08559.B6C8R48); COL9A3 (JHU08659.B8C4R44); ZUFSP (JHU08078.B6C20R42); NDE1 (JHU00337.B14C6R84); ANKRD17 (JHU12257.312C2R18); GNAS (JHU28981.B17C22R6); TTLL12 (JHU30284.B17C29R40); WRAP73 (JHU15189.B9C11R60); HSF2 (JHU19738.B14C4R2); CH25H (JHU04335.B4C32R72); TCAP (JHU01718.B4C9R26); STK24 (JHU16471.B10C20R80); EDIL3 (JHU06744.B6C6R14); CCDC155 (JHU09052.B5C16R52); SLC2A14 (JHU06795.B5C11R14); FCHO1 (JHU12286.B12C23R16); GAS2L1 (JHU12862.B15C32R86); HSF2 (JHU19738.B17C24R10); CCNI (JHU04718.B4C32R74); FANCE (JHU10008.B8C26R70); ATP8B5P_frag (JHU15426.B11C27R66); TNFRSF1A (JHU18512.B15C18R46); DAXX (JHU11066.B7C31R84); SLC27A3 (JHU08714.B8C16R46); CLPTM1 (JHU08558.B6C17R48); HSF2 (JHU26287.B19C23R6); CD74 (JHU01357.B3C1R22); CPEB1 (JHU06736.B6C11R14); APBA1 (JHU18056.B15C31R62); REST (JHU16767.B16C4R40); MPRIP (JHU19494.B13C14R30); TCAP (JHU01625.B4C32R42); OSBPL6 (JHU06594.B5C15R18); PTGS2 Auto-antigen.B20C24R38); TAGLN2 (JHU06128.B8C13R6); BC089418 (JHU16002.B10C10R72); OGFOD3 (JHU15317.B11C20R64); NAF1 (JHU03983.B13C20R80); EOGT (JHU10282.B6C10R68); HARS (JHU05318.B4C30R84); DCDC2 (JHU13615.B9C19R32); BCL2L13 (JHU01262.B4C27R24); CORO6 (JHU09909.B13C26R90); CRTAC1 (JHU03192.B4C32R50); BAFF (JHU29136.B20C1R38); DEFB132 (JHU13092.B12C4R90); KCNAB2 (JHU07150.B6C16R20); MMD2 (JHU15539.B12C25R62); ABTB1 (JHU08641.B8C16R48); PDCD2 (JHU13853.B11C20R38); HAT1_frag (JHU01484.B3C7R22); CPEB3 (JHU12849.B13C23R74); THEMIS2_frag (JHU15201.B12C7R56); and GPBPILI (JHU04644.B3C23R74).

### Conclusion:

Samples of humanised 7B1F3 and mouse 7B1F3 both recognised a peptide with the sequence TDVLNQSKPVTL (“Peptide 3”) as the top hit with very high specificity.

In particular, FIGS. 28 and 29 show that there is an essentially undetectable cross-reactivity, for both tested antibodies, with either HSPD1 (also known as HSP60) and ITGA6 (integrin alpha 6, also known as CD49f). Indeed, when ordering the list of over 23,000 protein targets by Z-score, HSPD1 (JHU02056.B15C21R82) was the 1,417^{th} protein; and ITGA6 (JHU11750.B10C1R6) was the 13,749^{th} protein.

These data show that even off-targets with a potential risk of cross-reactivity with the ECD1 epitope are not bound by the 7B1F3 clone.

### Example 5

### Retrogenix Cell Microarray

### Aim:

The aim of this Study is to use the ‘Cut-down’ version of the Retrogenix Cell Microarray technology platform to screen exemplary binding molecules for specific off-target binding.

The exemplary binding molecules tested are two alternative formats of bispecific T-cell engaging (BiTE) molecule, each possessing the V_{H} and V_{L} chains of 7B1F3 as described herein which thus bind to an epitope within the ECD1 region of HCMV-encoded US28 as further defined by SEQ ID NO: 177, and OKT3 V_{H} and V_{L} domains which bind the T-cell receptor CD3. The exemplary BiTEs used in this example are referred to herein as Thbi44.7_bis (molecular weight 1.73 kDa) and Thbi44.8_bis (molecular weight 150 kDa), and their structures are shown in FIG. 38. These are both in a bispecific IgG1, Fc silenced format. The species of the IgG and Fc is human. The screening concentration used was 0.1 μg/mL.

### Background:

The Retrogenix Cell Microarray technology is suited to screening for interactions of protein ligands and antibodies to human plasma membrane and secreted proteins, each of which is individually expressed in human cells. It makes use of a vast set of expression vectors, encoding human plasma membrane proteins, tethered human secreted proteins and heterodimer plasma membrane protein complexes.

The technology can a) identify the receptor/target for protein ligands and antibodies whose receptor is unknown, and b) identify any unwanted off-target activities for such molecules. The technology has proven extremely powerful for both applications.

In this Study, exemplary binding molecules Thbi44.7_bis and Thbi44.8_bis were screened against Discovery UK's most up to date library of human plasma membrane proteins, tethered human secreted proteins and heterodimer plasma membrane protein complexes to identify any potential off-target binding activity.

### Methodology:

i. Pre-Screen

Exemplary binding molecules were screened to determine optimal concentrations, on slides of fixed, untransfected HEK293 cells for assessment of background levels of binding (for example, due to high endogenous primary target expression in HEK293 cells). Binding was assessed using an appropriate and well-validated AlexaFluor647-labelled detection reagent, followed by imaging for fluorescence. The Pre-screen data included a quantitative assessment of backgrounds (fold increase over ‘no binding molecule’ treated slides). An optimal concentration of 0.1 μg/ml was determined.

### B. Library Screen

For Library screening, the test antibodies were screened for binding against 6052 human plasma membrane proteins (referred to herein as “List C”), secreted and cell surface-tethered human secreted proteins+397 human heterodimers (referred to herein as “List D”) expressed in human HEK293 cells using a cut-down version of the Retrogenix Cell Microarray. The full list of the different proteins of List C and List D as used in this Example are provided as “List C” and “List D”, respectively, in the Research Disclosures publication (ISSN 0374-4353, Questel Ireland Ltd) with database number 707020, entitled “Large scale protein arrange target protein identities”, digitally published on 27 Jan. 2023, the contents which are incorporated herein by reference in their entirety, for the purposes of the disclosure of the proteins defined by “List C” and “List D”.

The library of expression vectors was arrayed in duplicate on microarray slides (“slide-sets”), respectively. An expression vector (pIRES-hEGFR-IRES-ZsGreen1) was spotted in quadruplicate on every slide and used to ensure that a minimal threshold of transfection efficiency was achieved or exceeded on every slide. This minimal threshold (mean ZsGreen1 signal from the pIRES-EGFR-ZsGreen1 vector over background) has been defined previously. Human HEK293 cells were used for reverse transfection/expression. The exemplary binding molecules were each applied individually at 0.1 μg/ml to all fixed slide sets (n=1 slide per slide set). Detection of binding was performed as per the Pre-screen. Fluorescent images were analysed and quantitated (for transfection efficiency) using ImageQuant software (GE).

A protein ‘hit’ is defined as duplicate spots showing a raised signal compared to background levels. This is achieved by visual inspection using the images gridded on the ImageQuant software. Hits were classified as ‘strong, medium, weak or very weak’, depending on the intensity of the duplicate spots.

### Results:

Thbi44.7_bis and Thbi44.8_bis were screened for binding against fixed HEK293 cells/slides expressing duplicate 6052 human plasma membrane proteins, secreted and cell surface tethered human secreted proteins+397 human heterodimers (18 slide sets, n=1 slide per slide set per antibody).

All transfection efficiencies exceeded the minimum threshold.

Test antibodies Thbi44.7_bis and Thbi44.8_bis were screened for binding against List C 6052 human plasma membrane proteins secreted and cell surface-tethered human secreted proteins+the List D 397 human heterodimers expressed in human HEK293 cells using a cut-down version of the Retrogenix Cell Microarray.

Excluding non-specific and low confidence (very weak intensity) interactions, no significant specific interactions were identified for Thbi44.7_bis or Thbi44.8_bis.

### Example 6

### ECD1 Humanisation and Aglycosylation Analysis

### Summary of Humanisation Method:

The parental V_{H} and V_{L} sequences of antibodies 4H3C3 and 7B1F3 were aligned with a panel of human germline sequences. Two closely aligned germline sequences were selected, and the CDRs and certain framework amino acids were grafted from the donor parental sequences onto the human acceptor germline sequence. In total, four V_{H} and four V_{L} sequences were produced, giving a possible combination of 16 antibodies. Percentage identity to human is calculated using the IMGT Domain Gap Align tool.

### Binding Analysis:

ii. Materials:
Cells: HEK Phoenix Ampho (ATCC-CRL-3213), p 21.
Staining buffer: PBS with 1% BSA, 2 mM EDTA.
Plasmid: Encoding US28 protein from different HCMV strains: DB; TB40/E; Merlin; and VHL/E.
Blocking: Human TruStain FcX™ (ref 422302).
Live Dead: Invitrogen™ eBioscience™ Fixable Viability Dye eFluor™ 780 (ref 13539140).
Primary antibodies: 7B1F3 (6.76 nM=1 μg/ml); hum7B1F3 (6.76 nM=1 μg/ml).

### Conjugated Secondary Antibodies:

APC anti-human IgG Fc Antibody (ref 410712): 1:200.
APC anti-mouse IgG Fc (ref 405308): 2.5 μg/mL.

### Controls:

Viability dye.
FMO.
Secondary Ab:
   APC anti-human IgG Fc Antibody (ref 410712): 1:200.
   APC anti-mouse IgG Fc (ref 405308): 2.5 μg/mL.

### Seeding:

The day before transfection, HEK Ampho cells were seeded in a 1×6-well plate with 1×10⁶ live cells/well, 2 mL/well. Seeded 4 wells for HEKPhoenix_plasmids. Seeded 2 wells for HEKPhoenix_Mock transfected. Incubated the cells in a 37° C. incubator with >80% relative humidity and 5% CO₂.

### Cell Transfection:

After 1 day, cells will have adhered and reached >60% confluency. Removed 700 uL media from each well prior to transfection. Allowed X-tremeGENE™ HP DNA Transfection Reagent, DNA and diluent Opti-MEM® I Reduced Serum Medium to warm to +15° C. to +25° C., and vortexed gently. Placed 100 μL diluent in a sterile tube for each condition. Added 1 μg plasmid DNA. Pipetted gently to mix (DNA was not added for the mock condition):
DB (683.3 ng/μL): 1.46 μL
TB40/E (742.05 ng/μL): 1.35 μL
Merlin (70.78 ng/μL): 14.13 μL
VHL/E (51.87 ng/μL): 19.28 μL

Added 3 μL X-tremeGENE™ HP DNA Transfection Reagent to the diluted DNA. (3:1 ratio of reagent to DNA). Pipetted gently to mix, then:
1. Incubated for 20 min at +15° C. to +25° C.
2. Add ˜100 μL transfection complex to the cells in a dropwise manner.
3. Gently shook or swirled the wells or flasks to ensure even distribution over the entire plate.
4. Incubated cells for 48 hours before measuring protein expression.
More details can be seen at: https://www.sigmaaldrich.com/NO/en/technical-documents/protocol/cell-culture-and-cell-culture-analysis/transfection-and-gene-editing/xtg-cell-protocols #EK-293T.

### Antibody Binding and Preparation:

Antibody binding and preparation are summarised in Tables 12 and 13, respectively.

TABLE 12
Antibody binding

| | HEKPhoenix_{—} | HEKPhoenix_{—} | HEKPhoenix_{—} | HEKPhoenix_{—} | HEKPhoenix_{—} |
|---|---|---|---|---|---|
| Antibody | Mock | BD | TB40/E | Merlin | VHL/E |
| FMO (no | 1 | | | | |
| APC) | | | | | |
| Sec Ab | 2 | 3 | 4 | 5 | 6 |
| mouse | | | | | |
| 7B1F3 | 7 | 8 | 9 | 10 | 11 |
| Sec Ab | 12 | 13 | 14 | 15 | 16 |
| human | | | | | |
| hum7B1F3 | 17 | 18 | 19 | 20 | 21 |

TABLE 13
Antibody preparation

| | concentration | Ab | Viability | SB |
|---|---|---|---|---|
| Block | | 37.5 | | 3000 |
| Viability dye | 1:1000 | | 2 | 1998 |
| Sec anti-mouse | 2.5 ug/mL | 7.5 | | 592.5 |
| Sec anti-human | 1:200 | 3 | | 597 |
| 7B1F3 | 1 ug/ml = | 1 | | 1000 |
| | 6.76 nM | | | |
| hum7B1F3 | 1 ug/ml = | 1 | | 1000 |
| | 6.76 nM | | | |

### Method:

Used 0.25×10⁶ total cells per well, detached with versene.
1. Washed cells once in 200 μL SB if the cells are in media
2. Centrifuged cells at 300-400×g (1200 rpm), 3 min.
3. If using plate, flipped supernatant off into sink, or aspirated using pipette.
4. Blocking: Fc block, 50 μl buffer+0.625 μl Trustain FcX/well to block Fc receptors. Added before Ab.
5. Incubated at RT for 10 minutes.
6. Prepared antibodies to the required dilution in SB.
7. Washed 1× with 100 μL SB.
8. Added 50 μl Ab-SB solution per well (not for sec only or FMO).
9. Incubated 30 min on ice.
10. Prepared solution of secondary Ab at working dilution.
11. Added 100 μl staining buffer to wash cells, centrifuge at 300-400×g for 3 min at 4° C., and discard supernatant. Washed again with 150 μL SB.
12. Added 50 μl SB secondary per well or nothing for no staining. Incubated for 15 minutes on ice.
13. Added 100 μl staining buffer to wash cells, centrifuged at 300-400×g for 3 min at 4° C., and discard supernatant.
14. Resuspended cells in 200 μl SB and transferred cells to flow tubes for acquisition of samples.

### Humanisation of 4H3C3:

It is common for antibody sequences to contain deamidation and isomerisation motifs. However, the 4H3C3 sequences do not contain any motifs of particular concern. Humanised sequences of 4H3C3 are shown in Table 14.

TABLE 14
Humanised sequences of 4H3C3.

| Type | Name | Germline | Sequence | human |
|---|---|---|---|---|
| Parental | VH | — | QVQLQQSGAELAKPGASVKMSCKASGYIFTSY | 73.5% |
| | | | WMHWVKQRPGQGLEWIGYINPSTGYAEYNQKF | |
| | | | KDKATLTADKSSSTAYMQLSSLTSEDSAVYYC | |
| | | | ARLRFGSSGFAYWGQGTLVTVSS (SEQ ID | |
| | | | NO: 187) | |
| Humanised | VH1 | IGHV1- | QVQLVQSGAEVKKPGASVKVSCKASGYTFTSY | 85.7% |
| | | 46*0-1 | WMHWVRQAPGQGLEWIGYINPSTGYAEYNQKF | |
| | | | QGRATLTADKSSSTAYMELSSLRSEDTAVYYC | |
| | | | ARLREGSSGFAYWGQGTLVTVSS | |
| | | | (SEQ ID NO: 256) | |
| Humanised | VH2 | IGHV1- | QVQLVQSGAEVKKPGASVKVSCKASGYTFTSY | 89.8% |
| | | 46*0-1 | WMHWVRQAPGQGLEWMGYINPSTGYAEYNQKF | |
| | | | QGRVTMTADKSTSTAYMELSSLRSEDTAVYYC | |
| | | | ARLREGSSGFAYWGQGTLVTVSS (SEQ ID | |
| | | | NO: 257) | |
| Humanised | VH3 | IGHV1- | QVQLVQSGAEVKKPGASVKVSCKASGYTITSY | 83.7% |
| | | 18*0-1 | WMHWVRQAPGQGLEWIGYINPSTGYAEYNQKL | |
| | | | QGRATLTADKSSSTAYMELRSLRSDDTAVYYC | |
| | | | ARLRFGSSGFAYWGQGTLVTVSS (SEQ ID | |
| | | | NO: 258) | |
| Humanised | VH4 | IGHV1- | QVQLVQSGAEVKKPGASVKVSCKASGYTFTSY | 87.8% |
| | | 18*0-1 | WMHWVRQAPGQGLEWMGYINPSTGYAEYNQKL | |
| | | | QGRVIMTADKSTSTAYMELRSLRSDDTAVYYC | |
| | | | ARLRFGSSGFAYWGQGTLVTVSS (SEQ ID | |
| | | | NO: 259) | |
| Parental | VL | — | QIVLTQSPSSLAVSVGEKVIMSCKSSQSLLYS | 84.0% |
| | | | SNQKNYLAWYQQKPGQSPKLLIYWASTWESGV | |
| | | | PDRFTGSGSGTDFTLTISSVKAEDLAVYYCQQ | |
| | | | YYSFPLTFGAGTKLEIK (SEQ ID NO: | |
| | | | 189) | |
| Humanised | VL1 | IGKV4- | QIVLTQSPDSLAVSIGERATINCKSSQSLLYS | 95.0% |
| | | 1*01 | SNQKNYLAWYQQKPGQPPKLLIYWASTWESGV | |
| | | | PDRFSGSGSGTDFTLTISSLQAEDVAVYYCQQ | |
| | | | YYSFPLTFGGGTKLEIK (SEQ ID NO: | |
| | | | 260) | |
| Humanised | VL2 | IGKV4- | DIVLTQSPDSLAVSLGERATINCKSSQSLLYS | 96.0% |
| | | 1*01 | SNQKNYLAWYQQKPGQPPKLLIYWASTRESGV | |
| | | | PDRESGSGSGTDFTLTISSLQAEDVAVYYCQQ | |
| | | | YYSFPLTFGGGTKLEIK (SEQ ID NO: | |
| | | | 261) | |
| Humanised | VL3 | IGKV2- | QIVLTQSPLSLPVTPGEPASISCRSSQSLLYS | 84.0% |
| | | 28*01 | SNQKNYLAWYLQKPGQSPQLLIYWASTWESGV | |
| | | | PDRFSGSGSGIDFTLKISRVEAEDVGVYYCQQ | |
| | | | YYSFPLTFGGGTKLEIK (SEQ ID NO: | |
| | | | 262) | |
| Humanised | VL4 | IGKV2- | DIVITQSPLSLPVTPGEPASISCRSSQSLLYS | 85.1% |
| | | 28*01 | SNQKNYLAWYLQKPGQSPQLLIYWASTRESGV | |
| | | | PDRFSGSGSGTDFTLKISRVEAEDVGVYYCQQ | |
| | | | YYSFPLTFGGGTKLEIK (SEQ ID NO: | |
| | | | 263) | |

### Humanisation of 7B1F3:

The sequence liability analysis revealed that 7B1F3 contains an N-linked glycosylation site in the middle of CDRH1-3. This site has been emboldened in the sequences within Table 15. This is a potential liability as it may create an increased risk of product heterogeneity as well as changes in the glycosylation, potentially impacting activity if the glycan is directly involved in binding.

TABLE 15
Humanised sequences of 7B1F3.

| Type | Name | Germline | Sequence | human |
|---|---|---|---|---|
| Parental | VH | — | EVQLQQSGAELAKPGASVKMSCKASGYTFTNY | 68.4% |
| | | | WMHWVKQRPGQGLEWIGYVNPRTGYTEYNQKF | |
| | | | KDKATLTADKSSRTAYMQLSSLTSEDSAVYSC | |
| | | | ARIINGTGFAYWGQGTLVTVSA (SEQ ID | |
| | | | NO: 211 | |
| Humanised | VH1 | IGHV1- | QVQLVQSGAEVKKPGASVKVSCKASGYTFTNY | 81.6% |
| | | 46*0-1 | WMHWVRQAPGQGLEWIGYVNPRTGYTEYNQKF | |
| | | | QGRATLTADKSSRTAYMELSSLRSEDTAVYSC | |
| | | | ARILNGTGFAYWGQGTLVTVSS (SEQ ID | |
| | | | NO: 264) | |
| Humanised | VH2 | IGHV1- | QVQLVQSGAEVKKPGASVKVSCKASGYTFTNY | 87.8% |
| | | 46*0-1 | WMHWVRQAPGQGLEWMGYVNPRIGYTEYNQKF | |
| | | | QGRVTMTADKSTSTAYMELSSLRSEDTAVYYC | |
| | | | ARINGTGFAYWGQGTLVTVSS (SEQ ID | |
| | | | NO: 265) | |
| Humanised | VH3 | IGHV1- | QVQLVQSGAEVKKPGASVKVSCKASGYTFTNY | 78.6% |
| | | 18*0-1 | WMHWVRQAPGQGLEWIGYVNPRTGYTEYNQQL | |
| | | | QGRATLTADKSSRTAYMELRSLRSDDTAVYSC | |
| | | | ARTINGTGFAYWGQGTLVTVSS (SEQ ID | |
| | | | NO: 266) | |
| Humanised | VH4 | IGHV1- | QVQLVQSGAEVKKPGASVKVSCKASGYTFTNY | 84.7% |
| | | 18*0-1 | WMHWVRQAPGQGLEWMGYVNPRTGYTEYNQQL | |
| | | | QGRVTMTADKSTSTAYMELRSLRSDDTAVYYC | |
| | | | ARIINGTGFAYWGQGTLVTVSS (SEQ ID | |
| | | | NO: 267) | |
| Parental | VL | — | DIVLTQSPSSLTVSVGEKETMSCKSSQSLLYS | 81.2% |
| | | | SNQKNYLAWYQQKPGQSPKLLIYWASTRESGV | |
| | | | PDRFTGSGSGTDFTLAISSVKPEDLAIYYCQQ | |
| | | | YYSFPLTFGAGTKLEIK (SEQ ID NO: | |
| | | | 213) | |
| Humanised | VL1 | IGKV4- | DIVITQSPDSLAVSIGERATINCKSSQSLLYS | 95.0% |
| | | 1*01 | SNQKNYLAWYQQKPGQPPKLLIYWASTRESGV | |
| | | | PDRFSGSGSGTDFTLTISSLQAEDVAIYYCQQ | |
| | | | YYSFPLTFGGGTKLEIK (SEQ ID NO: | |
| | | | 268) | |
| Humanised | VL2 | IGKV4- | DIVLTQSPDSLAVSLGERATINCKSSQSLLYS | 96.0% |
| | | 1*01 | SNQKNYLAWYQQKPGQPPKLLIYWASTRESGV | |
| | | | PDRESGSGSGTDFTLTISSLQAEDVAVYYCQQ | |
| | | | YYSFPLTFGGGTKLEIK (SEQ ID NO: | |
| | | | 261) | |
| Humanised | VL3 | IGKV2- | DIVLTQSPLSLPVIPGEPASISCRSSQSLLYS | 84.2% |
| | | 28*01 | SNQKNYLAWYLQKPGQSPQLLIYWASTRESGV | |
| | | | PDRESGSGSGIDFTLKISRVEAEDVGIYYCQQ | |
| | | | YYSFPLTFGGGTKLEIK (SEQ IDNQ. | |
| | | | 269) | |
| Humanised | VL4 | IGKV2- | DIVETQSPLSLPVTPGEPASISCRSSQSLLYS | 85.1% |
| | | 28*01 | SNQKNYLAWYLQKPGQSPQLLIYWASTRESGV | |
| | | | PDRFSGSGSGTDFTLKISRVEAEDVGVYYCQQ | |
| | | | YYSFPLTFGGGTKLEIK (SEQ ID NO: | |
| | | | 263) | |

### Structural Analysis:

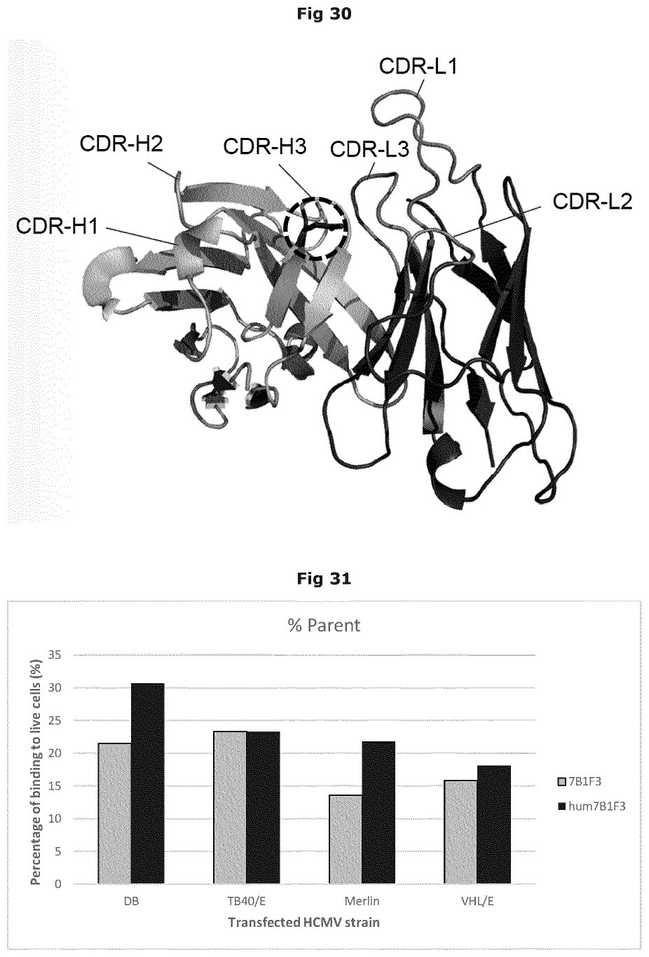

A structural analysis of the 7B1F3 clone was performed to observe the orientation of the N97 (i.e. the N in NGT that is the sequence liability for N-glycosylation). The structure of 7B1F3 is shown in FIG. 30.

Based on the structural analysis alone, it is not possible to know if the position of the N94 is critical for binding. However, based on the orientation, it was recognised that it may be possible to mutate the N-glycosylation site out, thereby reducing the risk associate with such a sequence liability.

### Aglycosylated Humanised Sequences:

Two further humanised V_{H} sequences have been designed with the asparagine (N) mutated to glutamine (Q) to remove the glycosylation site (Table 16). This mutation is highlighted in bold font in the table below. As this mutation may impact on target binding, this was subsequently assessed.

TABLE 16
Aglycosylated humanised sequences of 7B1F3.

| | | | | % |
|---|---|---|---|---|
| Type | Name | Germline | Sequence | human |
| Humanised | VH5 | IGHV1- | QVQLVQSGAEVKKPGASVKVSCKASGYTFTNY | 81.6% |
| | | 46*0-1 | WMHWVRQAPGQGLEWIGYVNPRTGYTEYNQKF | |
| | | | QGRATLTADKSSRTAYMELSSLRSEDTAVYSC | |
| | | | ARILQGTGFAYWGQGTLVTVSS (SEQ ID | |
| | | | NO: 270) | |
| Humanised | VH6 | IGHV1- | QVQLVQSGAEVKKPGASVKVSCKASGYTFTNY | 87.8% |
| | | 46*0-1 | WMHWVRQAPGQGLEWMGYVNPRTGYTEYNQKF | |
| | | | QGRVTMTADKSTSTAYMELSSLRSEDTAVYYC | |
| | | | ARTLQGTGFAYWGQGTLVTVSS (SEQ ID | |
| | | | NO: 271) | |

### Results:

The level of binding to the target was normalised by subtracting the background level of binding (as determined by the mock negative control, which are not transfected to express US28). The parental 7B1F3 and humanised 7B1F3 were then compared for binding to cells transfected with the various vectors.

The parent cells are the live cells detected in the total population observed following flow cytometry. Therefore, the % parent readout is reflective of the percentage of live cells that are positive for the binding of the 7B1F3 antibody (see FIG. 31).

Unexpectedly, the percentage of binding increased for the humanised antibody compared with the parental version for the DB, Merlin and VHL/E strains, while maintaining equivalent binding to the TB40/E strain.

### Conclusion:

These data demonstrate that humanisation of an exemplary ECD1 binding molecule can positively impact target binding for a number of US28 strains.

### Example 7

Aglycosylation Mutant of Mouse 7B1F3 Comparison with Parental Mouse 7B1F3

### Overview:

This example reports the comparative testing of the parental mouse 7B1F3 sequence, as described above, with an aglycosylation mutant thereof in which CDR3 of the V_{H} chain is mutated, to replace the asparagine (N) with glutamine (Q) (at the position corresponding to that shown in Table 16, above) to remove the glycosylation site. Binding was assessed against US28 forms encoded by different HCMV strains.

### Material Preparation:

Staining buffer: PBS with 0.5% BSA, 2 mM EDTA.
Blocking solution (cell number dependent): for 0.25×10⁶ cells:
   Human block: 0.625 μl Trustain FcX in 50 μl Staining buffer per well.
Viability dye solution: Viability dye diluted 1:1000 in Staining buffer.
Primary Ab solution: Dilute Ab accordingly in Staining buffer.
Secondary Ab solution: Dilute Ab 1:200 in Staining buffer.

| | | | Viability | Staining |
|---|---|---|---|---|
| | Concentration | Ab | dye | Buffer |
| Block | | 50 | | 4000 |
| Viability dye | 1:1000 | | 6 | 6000 |
| Secondary | 1:200 | 20 | | 4000 |
| anti-mouse | | | | |
| Ab | A 50 μg/ml | 17.5 | | 332.5 |
| | B 20 μg/ml | 7 | | 343 |
| | C 10 μg/ml | 3.5 | | 346.5 |
| | D 5 μg/ml | A 35 | | 315 |
| | E 2 μg/ml | B 35 | | 315 |
| | F 1 μg/ml | C 35 | | 315 |
| | G 0.5 μg/ml | A 3.5 | | 346.5 |
| | H 0.2 μg/ml | B 3.5 | | 346.5 |
| | I 0.1 μg/ml | C 3.5 | | 346.5 |

### Staining:

1. Plated 0.2×10⁶ cells per well in a 96-well plate (maximum volume of 200 μl).
2. Centrifuged cells at 300×g, 3 min at 4° C.
3. Flipped the supernatant off into the sink.
4. Washed cells by adding 150 μl of staining buffer.
5. Centrifuged cells at 300×g, 3 min at 4° C.
6. Blocking:
   Added 50 μl of blocking solution.
   Incubated at RT for 10 min.
7. After the incubation, added 100 μl staining buffer per well to wash the cells.
8. Centrifuged at 300×g for 3 min at 4° C., and discarded the supernatant.
9. Primary Ab/Viability staining:
   Added 50 μl Primary Ab/Viability dye solution per well.
   Added 50 μl Viability dye solution only per well that is not stained with primary Ab.
   Incubated for 30 min on ice.
10. Added 100 μl staining buffer to wash cells.
11. Centrifuged at 300×g for 3 min at 4° C., and discarded the supernatant.
12. Washed a second time.
13. Secondary Ab staining:
   Added 50 μl Secondary Ab solution per well.
   Incubated for 15 minutes on ice.
14. Added 100 μl staining buffer to wash cells.
15. Centrifuged at 300×g for 3 min at 4° C., and discarded the supernatant.
16. Repeated the washing step by adding 100 μl staining buffer, spin, and discarded the supernatant.
17. Resuspended cells in 200 μl staining buffer.
18. Transferred cells to flow tubes for the acquisition of samples.

### Compensation:

Cells not stained: Cells that are blocked but not stained (only staining buffer in all the other steps).

Cells+Viability dye: Cells are killed by incubating them at 95° C. for 2 min and then putting them directly in ice. The cells are blocked and then stained with viability dye only (staining buffer only in other steps).

Beads+Secondary Ab: Beads are stained following the protocol given by the manufacturer.

### Results:

The optimal concentration to assess differences in binding in this assay was determined to be 0.5 μg/ml. Using the results obtained at this concentration, the background binding observed in the mock negative control was subtracted from the percentage binding values for the various conditions. The resultant binding of the parental murine 7B1F3 antibody to each US28 strain was normalised at 100%, and the relative level of binding for the aglycosylated 7B1F3 is assessed against this.

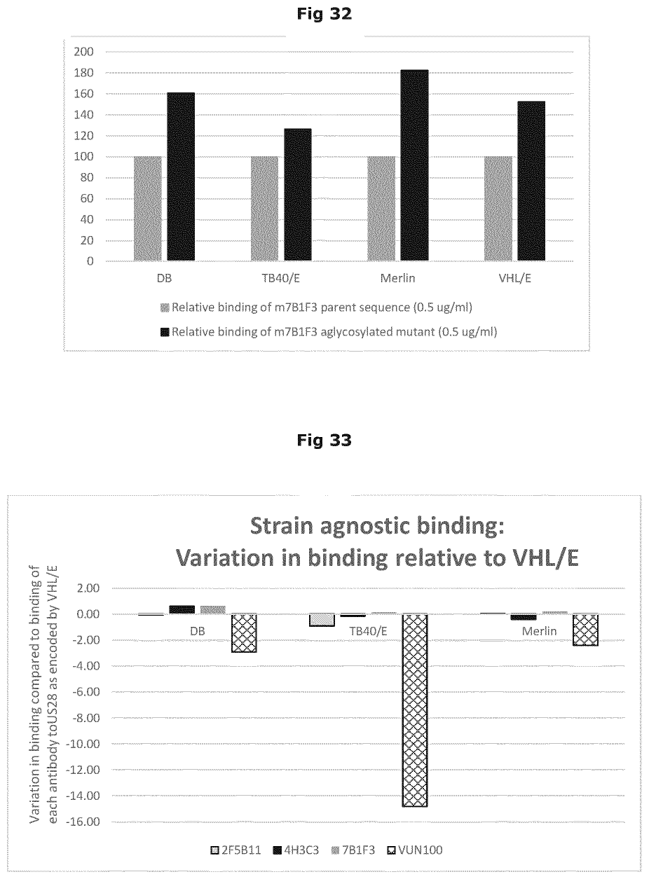

An increase in the percentage binding is observed for the aglycosylated 7B1F3 in comparison with the parental murine 7B1F3 (abbreviated to “m7B1F3”) for all HCMV strain variants of US28, as can be seen in FIG. 32.

### Conclusion:

These data demonstrate that sequence liabilities, such as N-glycosylation sites in CDR-H3, can be addressed by point mutation. In this case, the asparagine was mutated to a glutamine (which have similar properties as amino acids) to as means to reduce the risk, and the mutated, aglycosylated versions surprisingly maintained or improved target binding to US28, irrespective of the strain variant for the target protein.

### Example 8

### Strain Agnostic Binding

### Overview:

This example assesses some of the exemplary binding molecules of the present invention, the antibodies 2F5B11, 4H3C3 and 7B1F3, to determine the extent to which binding to US28 is maintained when the sequence of US28 is varied, based on the sequence variations of US28 encoded by HCMV strains TB40/E, DB, Merlin and VHL/E. By way of comparison, the prior art antibody VUN100 (using the bivalent form described by De Groof et al, 2020, supra) was assessed in the same assay.

### Materials:

Cells: HEK Phoenix Ampho (ATCC-CRL-3213).
Staining buffer: PBS with 0.5% BSA, 2 mM EDTA.
Plasmid: Encoding US28 protein from different HCMV strains: DB; TB40/E; Merlin; and VHL/E.
Blocking: Human TruStain FcX™ (ref 422302).
Live Dead: Invitrogen™ eBioscience™ Fixable Viability Dye eFluor™ 780 (ref 13539140).

### Primary Antibodies:

Each of the primary antibodies 7B1F3, 2F5B11, 4H3C3 and bivalent VUN100 (SEQ ID NO: 63) were labelled with a Myc tag for detection by a secondary antibody. Concentrations tested included 67.6 nM=10 ug/ml; 6.76 nM=1 ug/ml; 0.676 nM=0.1 ug/ml

### Conjugated Secondary Antibodies:

Alexa Fluor 647 anti-c-Myc Antibody (ref 626810): 1:200

### Controls:

Viability dye
FMO
Secondary Ab: Alexa Fluor 647 anti-c-Myc Antibody (ref 626810): 1:200

### Seeding:

The day before transfection, seed HEK Ampho in a 1×6-well plate with 1×10⁶ live cells/well, 2 mL/well. Seeded 8 wells for HEKPhoenix_plasmids and 2 wells for HEKPhoenix_Mock transfected. Incubated the cells in a 37° C. incubator with >80% relative humidity and 5% CO₂.

### Cell Transfection:

After 1 day, cells will have adhered and reached >60% confluency. Removed 700 uL media from each well prior to transfection.
Allowed X-tremeGENE™ HP DNA Transfection Reagent, DNA and diluent Opti-MEM® I Reduced Serum Medium to warm to +15° C. to +25° C., and vortexed gently.
Placed 100 μL diluent in a sterile tube for each condition.
Added 1 μg plasmid DNA. Pipetted gently to mix. (DNA not added for the mock condition):
   b. DB (683.3 ng/μL): 1.46 μL
   c. TB40/E (742.05 ng/μL): 1.35 μL
   d. Merlin (70.78 ng/μL): 14.13 μL
   e. VHL/E (51.87 ng/μL): 19.28 μL
Added 3 μl X-tremeGENE™ HP DNA Transfection Reagent to the diluted DNA. (3:1 ratio of reagent to DNA). Pipetted gently to mix.
   1. Incubated for 20 min at ±15° C. to +25° C.
   2. Added ˜100 μL transfection complex to the cells in a dropwise manner.
   3. Gently shook or swirled the wells or flasks to ensure even distribution over the entire plate.
   4. Incubated cells for 48 hours before measuring protein expression.
   https://www.sigmaaldrich.con/NO/en/technical-documents/protocol/cell-culture-and-cell-culture-analysis/transfection-and-gene-editing/xtg-cell-protocols #EK-293T.

### Method:

Used 0.25×10⁶ total cells per well, detached with trypsin.
1. Washed cells once in 200 μL SB if the cells are in media.
2. Centrifuged cells at 300-400×g (1200 rpm), 3 min.
3. If using plate, flipped supernatant off into sink, or aspirated using pipette.
4. Blocking: Fc block, 50 μL buffer+0.625 ul Trustain FcX/well to block Fc receptors. Should be added before Ab.
5. Incubated at RT for 10 minutes.
6. Prepared antibodies to the required dilution in SB.
7. Washed 1× with 100 μL SB.
8. Added 50 μL Ab-SB solution per well (not for sec only or FMO).
9. Incubated 30 min on ice.
10. Prepared solution of secondary Ab at working dilution.
11. Added 100 μL staining buffer to wash cells, centrifuged at 300-400×g for 3 min at 4° C., and discarded supernatant. Washed again with 150 uL SB.
12. Added 50 μL SB secondary per well or nothing for no staining. Incubated for 15 minutes on ice.
13. Added 100 μL staining buffer to wash cells, centrifuged at 300-400×g for 3 min at 4° C., and discarded supernatant.
14. Resuspended cells in 200 μL SB and transferred cells to flow tubes for acquisition of samples.

### Results:

Flow cytometry results indicated that an antibody concentration of 6.76 nM was optimal for this analysis. FIG. 33 shows the relative variation in binding of each antibody, using the level of binding to the US28 variant encoded by HCMV strain VHL/E as a reference point. Thus:
a variation of zero indicates no difference in binding of a given antibody as between the US28 variant encoded by HCMV strain VHL/E and the US28 variant encoded by a different HCMV strain;
a variation of greater than zero indicates an increase in binding relative to the level of binding to the VHL/E encoded US28; and
a variation less than zero indicates a decrease in binding relative to the level of binding to the VHL/E encoded US28.

The bivalent VUN100 antibody demonstrated a substantial decrease in binding to all other tested US28 variants (encoded by DB, TB40/E and Merlin strains), compared to the level of binding to the VHL/E-encoded US28 variant sequence. In contrast, the exemplary antibodies of the present invention (2F5B11, 4H3C3, and 7B1F3) showed only minor variations in the level of to all other tested US28 variants (encoded by DB, TB40/E and Merlin strains), compared to the level of binding to the VHL/E-encoded US28 variant sequence, thereby demonstrating that they provide greatly-improved strain agnostic binding characteristics compared to the VUN100 antibody.

The data underlying the plot in FIG. 33 are given in Tables 17A & B, below.

TABLE 17A
Preliminary Binding Data.

| Antibody | Mock | VHL/E | DB | TB40/E | Merlin |
|---|---|---|---|---|---|
| 2F5811-Myc | 0.015 | 1.08 | 1.04 | 0.57 | 1.09 |
| 4H3C3-Myc | 0.015 | 0.16 | 0.37 | 0.14 | 0.12 |
| 781F3-Myc | 0.06 | 0.37 | 0.91 | 0.42 | 0.45 |
| VUN100bv-Myc | 0.031 | 2.29 | 0.62 | 0.16 | 0.71 |

Based on the preliminary data in Table 17A, the binding value for each antibody against the ‘mock’ (i.e. negative control) sample can be subtracted from the binding value against the US28 variant from each of the VHL/E, DB, TB40/E and Merlin strains to calculate the extent of US28-specific binding for each antibody, against each strain. For the purposes of this analysis, and to allow cross-comparisons, the values are then normalised against the observed binding of one of the US28 variants; here the values were normalised against the US28 protein as encoded by the VHL/E strain. The fold-difference is then calculated (wherein a fold-difference of 1 is no change and greater than 1 is a change), and a value of 1 subtracted (so that a variation of 0 is no change, and a variation different to 0 is a change). Finally, for the benefit of a clear visual representation in FIG. 33, positive and negative values are assigned to indicate an increase or decrease in binding, relative to the level of binding observed against the US28 protein as encoded by the VHL/E strain. After being processed according to the foregoing approach, the data plotted in FIG. 33 are shown in Table 17B.

TABLE 17B
Processed Binding Data.

| | Variation in binding, relative to determined binding to US28 | | | |
|---|---|---|---|---|
| | as encoded by VHL/E | | | |
| Antibody | VHL/E | DB | TB40/E | Merlin |
| 2F5B11-Myc | 0 | −0.04 | −0.92 | +0.01 |
| 4H3C3-Myc | 0 | +0.59 | −0.16 | −0.38 |
| 7B1F3-Myc | 0 | +0.64 | +0.14 | +0.21 |
| VUN100bv- | 0 | −2.92 | −14.83 | −2.41 |
| Myc | | | | |

### Conclusions:

As shown in FIG. 33, the present assay demonstrates that there is only a low level of variation in the observed binding for exemplary binding molecules of the present invention (7B1F3, 2F5B11, 4H3C3), when their binding is compared to the different variant US28 proteins encoded by HCMV strains VHL/E, DB, TB40/E and Merlin.

These results contrast with the far greater level of variation in the observed binding for bivalent VUN100, when its binding is compared to the same group of variant US28 proteins encoded by HCMV strains VHL/E, DB, TB40/E and Merlin. This is consistent with previous reports of the binding of VUN100 to the different HCMV strains. For example, FIG. 8D of WO 2019/151865 showed differences in binding between the VHL/E, Merlin and TB40/E strains of HCMV, with binding being particularly reduced in strain TB40/E (1 type), which is also the lowest bound variant by bivalent VUN100 in the present results.

A key difference between the exemplary binding molecules of the present invention (7B1F3, 2F5B11, 4H3C3) and the bivalent VUN100 antibody of the prior art is the location of the epitope within the US28 protein that is bound.

As discussed above, VUN100 has shown to bind to a discontinuous epitope, which comprise multiple binding positions within the N-terminal extracellular region of US28. De Groof (2019, supra) identified that removal of the N-terminal amino acid positions 1-22 results in no binding, and further identified that VUN100 does not bind to amino acid positions 11-15, but that amino acid position 16 is required.

However, the applicant determined that there is a high degree of sequence divergence in the region of US28 bound by VUN100 (see Table 3 of the present application), and has further surprisingly identified that, unlike many of the areas of the N-terminal region (ECD1) of the US28 protein of HCMV, the amino acid sequence of TDVLNQSKPVTL (SEQ ID NO: 177; as bound by binding molecules of the present invention) is highly conserved between all known HCMV strains, and furthermore is suitably immunogenic. As such the SEQ ID NO: 177 sequence (and immunogenic fragment thereof) is suitable to use as an antigen for generating anti-US28 antibodies that have one or more (such as all) of: improved strain agnostic binding properties, improved (i.e. relatively higher) binding specificity, and/or improved (i.e. relatively lower) off-target binding activity, for example as compared to VUN100 antibodies of the prior art as discussed above.

The data in FIG. 33 validate the applicant's hypothesis that binding molecules of the present invention, which are directed to an epitope within a polypeptide consisting of the amino acid sequence of TDVLNQSKPVTL (SEQ ID NO: 177) have improved strain agnostic binding properties, for example as compared to VUN100 antibodies of the prior art as discussed above.

### Example 9

### Cell Killing Assay

### Objectives:

The aim of this study is the testing of bispecific T-cell engaging (BiTEs) which include binding regions of the exemplary ECD1-binding molecules of the present invention. The BiTEs used are Thbi44.7_bis and Thbi44.8_bis as described above in Example 5 and shown in FIG. 38.

The example makes use of the Cypre 3D Immuno-Oncology in vitro assay, using the cancer cell line models (one cell line expresses US28 and is termed “MAXF401”, and the cell line other does not express US28 and is termed “MDA-MB-231”) in co-culture with human dermal fibroblasts (HDF) and PBMCs (obtained from donors) in order to assess therapeutic effect of the tested BiTEs, as determined by tumor size.

Additional details of the methods used can be seen in Hribar et al., 2019 (“A Simple Three-dimensional Hydrogel Platform Enables Ex Vivo Cell Culture of Patient and PDX Tumors for Assaying Their Response to Clinically Relevant Therapies”, Mol Cancer Ther, 18(3), the disclosures of which are incorporated by reference).

### Study Outline:

In this assay, tumor cells of one PDX and one cell line model (MAXFTN 401 and MDA-MB-231, respectively) were embedded into layers of Cypre's VersaGel® (a biocompatible and chemically defined extracellular matrix hydrogel) using the Symphony® 3D gel patterning technology for standard 96-well plates. Fibroblasts were additionally co-cultured with the tumor cells to mimic the stromal region of the tumor microenvironment.

Cypre 3D Assay: After the 3D tumors were established, they were treated with test binding molecules, and additionally with unactivated PBMCs for immuno-oncology (IO) assays. PBMCs were initially added into the cell culture medium above the 3D gel; over time, they gradually infiltrate and target tumor spheres inside the gel in response to immunomodulators.

The assay effectively recapitulates the immune infiltration through the stroma that is seen in the physiological TME prior to tumor killing, delivering translationally relevant results. The PBMCs were used unactivated.

The following assay readout options were selected for the test binding molecules:

Tumor growth delay: High content confocal imaging is used to analyze tumor size reduction.

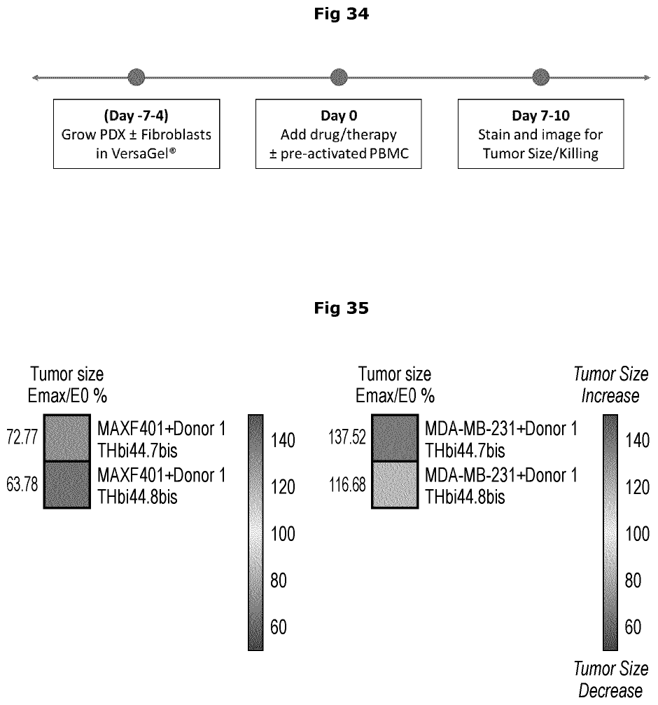

A typical assay timeline can be seen in FIG. 34.

### Assay Setup:

All compounds were tested in a 6-point, concentration response curve in duplicate. Compounds were serial diluted 1:4. Control drug—Staurosporine at one concentration. Supernatants were collected and stored at end of the study for further analysis. Endpoint is typically 4 days after compound treatment.

### Tumour Model Information/Cell Line Information:

| Tumour | Tumour | Cancer | Pre-implantation | Stage of | |
|---|---|---|---|---|---|
| designation | number | type | chemo/radiotherapy | Implantation | Histology |
| MAXFTN | MDA-MB-231 | Breast | Not known | Not available | Mammary |
| | | cancer | | | gland, |
| | | (triple | | | breast |
| | | negative) | | | |
| MAXFTN | 401 | Breast | Rad | M1 lung | Adeno |
| | | cancer | | | carcinoma |
| | | (triple | | | |
| | | negative) | | | |

| Primary/ | | | | | |
|---|---|---|---|---|---|
| Metastasis/ | Site of | | Patient age | | Ethnicity/ |
| Recurrent | origin | Differentiation | at surgery | Gender | Strain |
| Metastasis | Pleural | Poor | 51 | Female | Caucasian |
| | effusion | | | | |
| Metastasis | Lung | Good | 51 | Female | Caucasian |

### Compound Information:

| | Compound 1 | | Compound 2 | |
|---|---|---|---|---|
| Compound name | Thbi44.7 | | Thbi44.8 | |
| Aspect (powder or solution) | Solution | | Solution | |
| Conc. [mg/ml] if delivered as | 1 | mg/mL | 1 | mg/mL |
| solution | | | | |
| Shipment temperature [° C.] | −80° | C. | −80° | C. |
| Storage temperature, stock [° C.] | −20° | C. | −20° | C. |
| Light protected (stock) Y/N | N | | N | |
| Vehicle, precise formulation | 50% glycerol | | 50% glycerol | |
| instructions¹ | (ThermoFisher cat: | | (ThermoFisher cat: | |
| | 11433297) in 1X PBS | | 11433297) in 1X PBS | |
| | (ThermoFisher cat: | | (ThermoFisher cat: | |
| | 10010056) | | 10010056) | |
| Storage temperature, formulated | −20° | C. | −20° | C. |
| CPD [° C.] | | | | |
| Light protected (formulation) Y/N | N | | N | |
| Formulated CPD is stable for a | 1 | year | 1 | year |

period of . . .

¹Components of vehicles are to be indicated with source of supply and article no

### List of Reference Compounds:

| | | | | Immune | |
|---|---|---|---|---|---|
| | | | | cells | Immune cells |
| Model | | | | required | required |
| Colon | Oxaliplatin | Cetuximab | 5-FU | Bevacizumab | Pembrolizumab |
| Cancer | | | | | |
| NSCLC | Docetaxel | Cisplatin | Afatinib (or | Atezollizumab | Pembrolizumab |
| | | | other EGFR | | |
| | | | TKI) | | |
| Renal | Sunitinb | Pazopanib | Temsirolimus | Bevacizumab | Pembrolizumab |
| Cancer | | | | | |

### Results: =

Heat maps of tumour size can be seen in FIG. 35.

The MDA-MB-231 cell line does not express the target protein, US28. It is therefore used in these experiments as a control to determine whether any anti-tumour affects are non-specific for cancer cells irrespective of the expression of the HCMV-encoded US28 target of the tested BiTEs. The MAXF401 cell line expresses US28, and so harbours the target for the tested BiTEs.

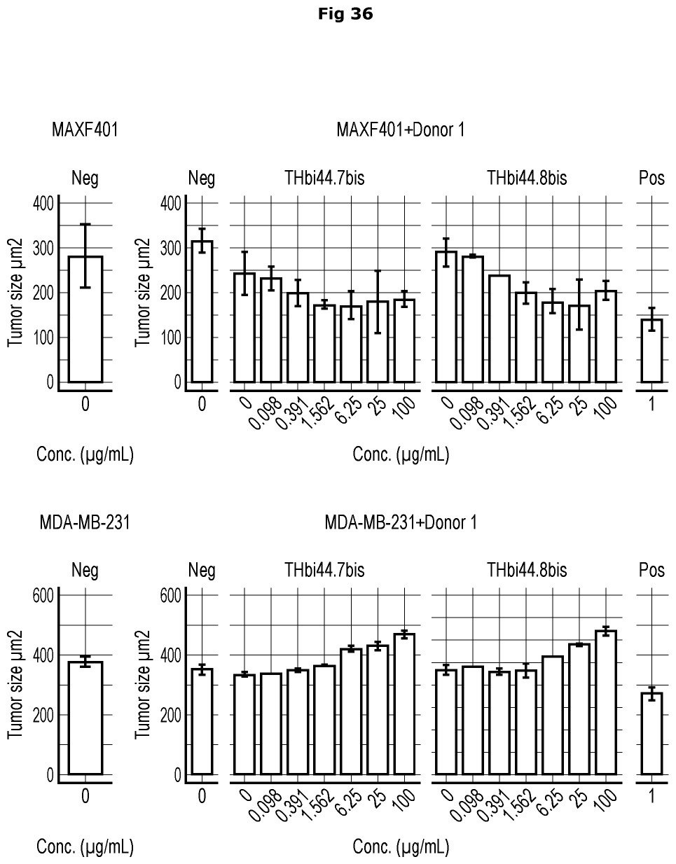

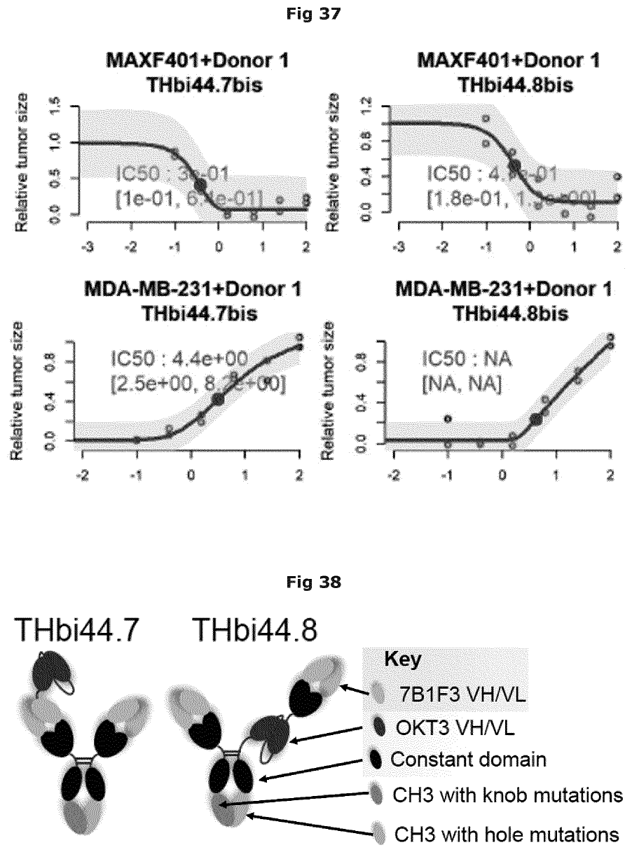

As can be seen in FIGS. 36 and 37, both exemplary ECD1-binding BiTE molecules caused a reduction in tumour size in a dose-dependent manner in the donor compared with the negative control, in the US28-expressing MAXF401 cell line.

In contrast, tumour size increases in the MDA-MB-231 cell line, which validates the hypothesis that the tested BiTEs are able to direct anti-cancer activity based on binding specificity to the HCMV-encoded US28 target. As such, the continued growth of the MDA-MB-231 cell line is not unexpected given that the tumour will continue to grow in the absence of the expression of a target for tested BiTEs.

Therefore, these data shows that reduction in tumour size is dependent on the expression of the US28 target protein for the ECD1-binding molecules, and further demonstrates that the BiTEs are able to successfully engage T-cells to mediate a targeted anti-cancer effect.

### REFERENCES FOR THE EXAMPLES

1. Georgiou, G., et al., The promise and challenge of high-throughput sequencing of the antibody repertoire. Nat Biotechnol, 2014. 32(2): p. 158-68.
2. Arav-Boger, R., et al., Polymorphisms of the cytomegalovirus (CMV)-encoded tumor necrosis factor-alpha and beta-chemokine receptors in congenital CMV disease. J Infect Dis, 2002. 186(8): p. 1057-64.
3. Groves, L J., M. B. Reeves, and J. H. Sinclair, Lytic infection of permissive cells with human cytomegalovirus is regulated by an intrinsic ‘pre-immediate-early’ repression of viral gene expression mediated by histone post-translational modification. J Gen Virol, 2009. 90(Pt 10): p. 2364-2374.
4. Elder, E. and J. Sinclair, HCMV latency: what regulates the regulators?Med Microbiol Immunol, 2019. 208(3-4): p. 431-438.
5. De Groof, T. W. M., et al., Nanobody-Targeted Photodynamic Therapy Selectively Kills Viral GPCR—Expressing Glioblastoma Cells. Mol Pharm, 2019. 16(7): p. 3145-3156.
6. Soroceanu, L., et al., Human cytomegalovirus US28 found in glioblastoma promotes an invasive and angiogenic phenotype. Cancer Res, 2011. 71(21): p. 6643-53.
7. Harkins, L. E., et al., Detection of human cytomegalovirus in normal and neoplastic breast epithelium. Herpesviridae, 2010. 1(1): p. 8.
8. Taher, C., et al., High prevalence of human cytomegalovirus in brain metastases of patients with primary breast and colorectal cancers. Transl Oncol, 2014. 7(6): p. 732-40.
9. Collins-McMillen, D., et al., Alternative promoters drive human cytomegalovirus reactivation from latency. Proc Natl Acad Sci USA, 2019. 116(35): p. 17492-17497.
10. Tarrant-Elorza, M., C. C. Rossetto, and G. S. Pari, Maintenance and replication of the human cytomegalovirus genome during latency. Cell Host Microbe, 2014. 16(1): p. 43-54.
11. Naucler, C. S., J. Geisler, and K. Vetvik, The emerging role of human cytomegalovirus infection in human carcinogenesis: a review of current evidence and potential therapeutic implications. Oncotarget, 2019. 10(42): p. 4333-4347.
12. Geisler, J., et al., A Review of the Potential Role of Human Cytomegalovirus (HCMV) Infections in Breast Cancer Carcinogenesis and Abnormal Immunity. Cancers (Basel), 2019. 11(12).
13. Elder, E., et al., Monocytes Latently Infected with Human Cytomegalovirus Evade Neutrophil Killing. iScience, 2019. 12: p. 13-26.
14. Hanahan, D. and R. A. Weinberg, Hallmarks of cancer: the next generation. Cell, 2011. 144(5): p. 646-74.
15. Kumar, A., et al., The Human Cytomegalovirus Strain DB Activates Oncogenic Pathways in Mammary Epithelial Cells. EBioMedicine, 2018. 30: p. 167-183.
16. Cobbs, C., Cytomegalovirus is a tumor—associated virus: armed and dangerous. Curr Opin Virol, 2019. 39: p. 49-59.
17. Adamson, C. S. and M. M. Nevels, Bright and Early: Inhibiting Human Cytomegalovirus by Targeting Major Immediate—Early Gene Expression or Protein Function. Viruses, 2020. 12(1).
18. Krishna, B. A., et al., The Requirement for US28 During Cytomegalovirus Latency Is Independent of US27 and US29 Gene Expression. Front Cell Infect Microbiol, 2020. 10: p. 1.86.
19. Cai, Z. Z., et al., Human cytomegalovirus—encoded US28 may act as a tumor promoter in colorectal cancer. World J Gastroenterol, 2016. 22(9): p. 2789-98.
20. Kumar A, Tripathy M K, Pasquereau S, Al Moussawi F, Abbas W, Coquard L, et al. The Human Cytomegalovirus Strain DB Activates Oncogenic Pathways in Mammary Epithelial Cells. EBioMedicine (2018) 30:167-83. doi: 10.1016/j.ebioni.2018.03.015.
21. Sandy Haidar Ahmad, Sebastien Pasquereau, Ranim El Baba, Zeina Nehme, Clara Lewandowski and Georges Herbein. Distinct Oncogenic Transcriptomes in Human Mammary Epithelial Cells Infected With Cytomegalovirus. Frontiers in Immunology (2021) 12: Article 772160.
22. Liliana Soroceanu et al. Cancer Res. 2011 Nov. 1; 71(21):6643-53.
23. Boivin et al. J Clin Virol. 2012 March; 53(3):208-13.
24. Göhring et al., Computational and Structural Biotechnology Journal, 2015; 13:153-158.
25. Jung et al. BMC Infectious Diseases (2019) 19:388.
26. Hribar et al., 2019 (“A Simple Three-dimensional Hydrogel Platform Enables Ex Vivo Cell Culture of Patient and PDX Tumors for Assaying Their Response to Clinically Relevant Therapies”, Mol Cancer Ther, 18(3)).

SEQUENCES

| SEQ ID NO: | Sequence | Description |
|---|---|---|
| SEQ ID NO: 1 | MTPTTTTAELTTEFDYDEAATPCVFTDVLNQSKPVTL | The N-terminal extracellular domain 1 (ECD1) of |
| | | US28 as encoded by HCMV strain DB (Accession |
| | | number KT959235), corresponding to positions 1- |
| | | 37 of SEQ ID NO: 5 |
| SEQ ID NO: 2 | QYLLDHNSLAS | The extracellular domain 2 (ECD2) of US28 as |
| | | encoded by HCMV strain DB (Accession number |
| | | KT959235), corresponding to positions 91-101 of |
| | | SEQ ID NO: 5 |
| SEQ ID NO: 3 | TKKNNQCMTDYDYLEVS | The extracellular domain 3 (ECD3) of US28 as |
| | | encoded by HCMV strain DB (Accession number |
| | | KT959235), corresponding to positions 167-183 |
| | | of SEQ ID NO: 5 |
| SEQ ID NO: 4 | VDTLKLLKWISSSCEFERSLKRAL | The extracellular domain 4 (ECD4) of US28 as |
| | | encoded by HCMV strain DB (Accession number |
| | | KT959235), corresponding to positions 250-273 |
| | | of SEQ ID NO: 5 |
| SEQ ID NO: 5 | MTPTTTTAELTTEFDYDEAATPCVFTDVLNQSKPVTLFLYGVVFLFGSIGNFLVIFTITWRRRIQCSGDVYF | The full sequence of US28 as encoded by HCMV |
| | INLAAADLLFVCTLPLWMQYLLDANSLASVPCTLLTACFYVAMFASLCFITEIALDRYYAIVYMRYRPVKQA | strain DB (Accession number KT959235) |
| | CLFSIFWWIFAVIIAIPHFMVVTKKNNQCMTDYDYLEVSYPITLNVELMLGAFVIPLSVISYCYYRISRIVA | |
| | VSQSRAKGRIVRVLIAVVLVFIIFWLPYALTLFVDTLKLLKWISSSCEFERSLKRALILTESLAFCHCCLNP | |
| | LLYVFVGTKFRQELHCLLAEFRQRLFSRDVSWYHSMSFSRRSSPSRRETSSDTLSDEVCRVSQIIP | |
| SEQ ID NO: 6 | TKKNNQCMTDYDYLEVS | ECD3 of the US28 protein as encoded by the |
| | | ECD3 4N variant group of HCMV strains (e.g. |
| | | strain DB, accession number KT959235) |
| SEQ ID NO: 7 | TKKDNQCMTDYDYLEVS | ECD3 of the US28 protein as encoded by the |
| | | ECD3 4D variant group of HCMV strains (e.g. |
| | | strain AF1, accession number GU179291.1) |
| SEQ ID NO: 8 | GFTFTDYY | V_{H}-CDR1 sequence of 1D3 |
| SEQ ID NO: 9 | IRSKANGYTT | V_{H}-CDR2 sequence of 1D3 |
| SEQ ID NO: 10 | ARDERRTAWLAY | V_{H}-CDR3 sequence of 1D3 |
| SEQ ID NO: 11 | MKWWLNWIFLVTLLNGIQCEVKLVESGGGLVQPGGSLRLACATSGFTFTDYYMSWVRQPPGKALEWLGFIRS | The immature form of the recombinantly |
| | KANGYTTEYSASVKGRFTISRDNSQSILYLQMNTLRSEDSATYYCARDERRTAWLAYWGQGTLVTVSA | expressed VH sequence of 1D3, wherein positions |
| | | 1-19 correspond to a leader sequence |
| SEQ ID NO: 12 | EVKLVESGGGLVQPGGSLRLACATSGFTFTDYYMSWVRQPPGKALEWLGFIRSKANGYTTEYSASVKGRFTI | The mature form of V_{H} sequence of 1D3, without |
| | SRDNSQSILYLQMNTLRSEDSATYYCARDERRTAWLAYWGQGTLVTVSA | the leader sequence |
| SEQ ID NO: 13 | MKLWLNWIFLVTLLNGIQC | The leader sequence of recombinantly expressed |
| | | V_{H} sequence of 1D3 |
| SEQ ID NO: 14 | QSIVHSNGNTY | V_{L}-CDR1 sequence of 1D3 |
| SEQ ID NO: 15 | KVS | V_{L}-CDR2 sequence of 1D3 |
| SEQ ID NO: 16 | FQGSHVPTWT | V_{L}-CDR3 sequence of 1D3 |
| SEQ ID NO: 17 | MKLPVRLLVLMVWIPASSSDVLMTQTPLSLPVSLGDQASISCRSSQSIVHSNGNTYLDWYLQKPGQSPKLLI | The immature form of the recombinantly |
| | YKVSNRFSGVPDRFSGSGSGTDFTLKISRVEAEDLGVYYCFQGSHVPTWTFGGGTKLEIK | expressed V_{L} sequence of 1D3, wherein positions |
| | | 1-19 correspond to a leader sequence |
| SEQ ID NO: 18 | DVLMTQTPLSLPVSLGDQASISCRSSQSIVHSNGNTYLDWYLQKPGQSPKLLIYKVSNRFSGVPDRFSGSGS | The mature form of V_{L} sequence of 1D3, without |
| | GTDFTLKISRVEAEDLGVYYCFQGSHVPTWTFGGGTKLEIK | the leader sequence |
| SEQ ID NO: 19 | MKLPVRLLVLMVWIPASSS | The leader sequence of recombinantly expressed |
| | | V_{L} sequence of 1D3 |
| SEQ ID NO: 20 | MKLWLNWIFLVTLLNGIQCEVKLVESGGGLVQPGGSLRLACATSGFTFTDYYMSWVRQPPGKALEWLGFIRS | The complete sequence of the heavy chain of |
| | KANGYTTEYSASVKGRFTISRDNSQSILYLQMNTLRSEDSATYYCARDERRTAWLAYWGQGTLVTVSAAKTT | 1D3. The leader sequence is underlined; it is |
| | PPSVYPLAPGSAAQTNSMVTLGCLVKGYFPEPVTVTWNSGSLSSGVHTFPAVLQSDLYTLSSSVTVPSSTWP | removed in the mature sequence. The mature V_{H} |
| | SETVTCNVAHPASSTKVDKKIVPRDCGCKPCICTVPEVSSVFIFPPKPKDVLTITLTPKVTCVVVDISKDDP | sequence is in bold. |
| | EVQESWFVDDVEVHTAQTQPREEQFNSTFRSVSELPIMHQDWLNGKEFKCRVNSAAFPAPIEKTISKTKGRP | |
| | KAPQVYTIPPPKEQMAKDKVSLTCMITDFFPEDITVEWQWNGQPAENYKNTQPIMDTDGSYFIYSKLNVQKS | |
| | NWEAGNTFTCSVLHEGLENHHTEKSLSHSPGK | |
| SEQ ID NO: 21 | MKLPVRLLVLMVWIPASSSDVLMTQTPLSLPVSLGDQASISCRSSQSIVHSNGNTYLDWYLQKPGQSPKLLI | The complete sequence of the light chain of 1D3. |
| | YKVSNRFSGVPDRFSGSGSGTDETLKISRVEAEDLGVYYCFQGSHVPTWTFGGGTKLEIKRADAAPTVSIFP | The leader sequence is underlined; it is removed |
| | PSSEQLTSGGASVVCFLNNFYPKDINVKWKIDGSERQNGVLNSWTDQDSKDSTYSMSSTLTLTKDEYERHNS | in the mature sequence. The mature V_{L} sequence |
| | YTCEATHKTSTSPIVKSFNRNEC | is in bold |
| SEQ ID NO: 22 | GGGTTCACCTTCACTGATTACTAT | Nucleic acid sequence encoding the V_{H}-CDR1 |
| | | sequence of 1D3 as defined by SEQ ID NO: 8 |
| SEQ ID NO: 23 | ATTAGAAGCAAAGCTAATGGTTACACAACA | Nucleic acid sequence encoding the V_{H}-CDR2 |
| | | sequence of 1D3 as defined by SEQ ID NO: 9 |
| SEQ ID NO: 24 | GCAAGAGATGAGCGCCGTACTGCCTGGCTTGCTTAC | Nucleic acid sequence encoding the V_{H}-CDR3 |
| | | sequence of 1D3 as defined by SEQ ID NO: 10 |
| SEQ ID NO: 25 | ATGAAGTTGTGGCTGAACTGGATTTTCCTTGTAACACTTTTAAATGGTATCCAGTGTGAGGTGAAGCTGGTG | Nucleic acid sequence encoding the immature |
| | GAGTCTGGAGGAGGCTTGGTACAGCCGGGGGGTTCTCTGAGACTCGCCTGTGCAACTTCTGGGTTCACCTTC | form of the recombinantly expressed V_{H} |
| | ACTGATTACTATATGAGCTGGGTCCGCCAGCCTCCAGGAAAGGCACTTGAGTGGCTGGGTTTTATTAGAAGC | sequence of 1D3 as defined by SEQ ID NO: 11. |
| | AAAGCTAATGGTTACACAACAGAGTACAGTGCATCTGTTAAGGGTCGGTTCACCATCTCCAGAGATAATTCC | The nucleic sequence encoding the leader |
| | CAAAGCATCCTCTATCTTCAAATGAACACCCTGAGATCTGAGGACAGTGCCACTTATTACTGTGCAAGAGAT | sequence is underlined |
| | GAGCGCCGTACTGCCTGGCTTGCTTACTGGGGCCAAGGGACTCTGGTCACTGTCTCTGCAGC | |
| SEQ ID NO: 26 | GAGGTGAAGCTGGTGGAGTCTGGAGGAGGCTTGGTACAGCCGGGGGGTTCTCTGAGACTCGCCTGTGCAACT | Nucleic acid sequence encoding the mature form |
| | TCTGGGTTCACCTTCACTGATTACTATATGAGCTGGGTCCGCCAGCCTCCAGGAAAGGCACTTGAGTGGQTG | of VH sequence of 1D3, without the leader |
| | GGTTTTATTAGAAGCAAAGCTAATGGTTACACAACAGAGTACAGTGCATCTGTTAAGGGTCGGTTCACCATC | sequence, as defined by SEQ ID NO: 12 |
| | TCCAGAGATAATTCCCAAAGCATCCTCTATCTTCAAATGAACACCCTGAGATCTGAGGACAGTGCCACTTAT | |
| | TACTGTGCAAGAGATGAGCGCCGTACTGCCTGGCTTGCTTACTGGGGCCAAGGGACTCTGGTCACTGTCTCT | |
| | GCAGC | |
| SEQ ID NO: 27 | ATGAAGTTGTGGCTGAACTGGATTTTCCTTGTAACACTTTTAAATGGTATCCAGTGT | Nucleic acid sequence encoding the leader |
| | | sequence of recombinantly expressed V_{H} |
| | | sequence of 1D3, as defined by SEQ ID NO: 13 |
| SEQ ID NO: 28 | CAGAGCATTGTACATAGTAATGGAAACACCTAT | Nucleic acid sequence encoding the V_{L}-CDR1 |
| | | sequence of 1D3 as defined by SEQ ID NO: 14 |
| SEQ ID NO: 29 | AAAGTTTCC | Nucleic acid sequence encoding the V_{L}-CDR2 |
| | | sequence of 1D3, as defined by SEQ ID NO: 15 |
| SEQ ID NO: 30 | TTTCAAGGTTCACATGTTCCCACGTGGACG | Nucleic acid sequence encoding the V_{L}-CDR3 |
| | | sequence of 1D3, as defined by SEQ ID NO: 16 |
| SEQ ID NO: 31 | ATGAAGTTGCCTGTTAGGCTGTTGGTGCTGATGGTCTGGATTCCTGCTTCCAGCAGTGATGTTTTGATGACC | Nucleic acid sequence encoding the immature |
| | CAAACTCCACTCTCCCTGCCTGTCAGTCTTGGAGATCAAGCCTCCATCTCTTGCAGATCTAGTCAGAGCATT | form of the recombinantly expressed V_{L} sequence |
| | GTACATAGTAATGGAAACACCTATTTAGACTGGTACCTTCAGAAACCAGGCCAGTCTCCAAAGCTCCTGATC | of 1D3 as defined by SEQ ID NO: 17. The |
| | TACAAAGTTTCCAACCGATTTTCTGGGGTCCCAGACAGGTTCAGTGGCAGTGGATCAGGGACAGATTTCACA | nucleic sequence that is underlined encodes |
| | CTCAAAATCAGCAGAGTGGAGGCTGAGGATCTGGGAGTTTATTACTGCTTTCAAGGTTCACATGTTCCCACG | positions 1-19 of the amino acid sequence of |
| | TGGACGTTCGGTGGAGGCACCAAGCTGGAAATCAAA | SEQ ID NO: 17, which correspond to the leader |
| | | sequence. |
| SEQ ID NO: 32 | GATGTTTTGATGACCCAAACTCCACTCTCCCTGCCTGTCAGTCTTGGAGATCAAGCCTCCATCTCTTGCAGA | Nucleic acid sequence encoding the mature form |
| | TCTAGTCAGAGCATTGTACATAGTAATGGAAACACCTATTTAGACTGGTACCTTCAGAAACCAGGCCAGTCT | of V_{L} sequence of 1D3, without the leader |
| | CCAAAGCTCCTGATCTACAAAGTTTCCAACCGATTTTCTGGGGTCCCAGACAGGTTCAGTGGCAGTGGATCA | sequence, as defined by SEQ ID NO: 18 |
| | GGGACAGATTTCACACTCAAAATCAGCAGAGTGGAGGCTGAGGATCTGGGAGTTTATTACTGCTTTCAAGGT | |
| | TCACATGTTCCCACGTGGACGTTCGGTGGAGGCACCAAGCTGGAAATCAAA | |
| SEQ ID NO: 33 | ATGAAGTTGCCTGTTAGGCTGTTGGTGCTGATGGTCTGGATTCCTGCTTCCAGCAGT | Nucleic acid sequence encoding the leader |
| | | sequence of recombinantly expressed V_{L} |
| | | sequence of 1D3, as defined by SEQ ID NO: 19 |
| SEQ ID NO: 34 | ATGAAGTTGTGGCTGAACTGGATTTTCCTTGTAACACTTTTAAATGGTATCCAGTGTGAGGTGAAGCTGGTG | Nucleic acid sequence encoding the complete |
| | GAGTCTGGAGGAGGCTTGGTACAGCCGGGGGGTTCTCTGAGACTCGCCTGTGCAACTTCTGGGTTCACCTTC | sequence of the heavy chain of 1D3, as defined |
| | ACTGATTACTATATGAGCTGGGTCCGCCAGCCTCCAGGAAAGGCACTTGAGTGGCTGGGTTTTATTAGAAGC | by SEQ ID NO: 20. The nucleic sequence that |
| | AAAGCTAATGGTTACACAACAGAGTACAGTGCATCTGTTAAGGGTCGGTTCACCATCTCCAGAGATAATTCC | is underlined encodes the leader sequence, |
| | CAAAGCATCCTCTATCTTCAAATGAACACCCTGAGATCTGAGGACAGTGCCACTTATTACTGTGCAAGAGAT | which is removed in the mature expressed |
| | GAGCGCCGTACTGCCTGGCTTGCTTACTGGGGCCAAGGGACTCTGGTCACTGTCTCTGCAGCCAAAACGACA | protein. |
| | CCCCCATCTGTCTATCCACTGGCCCCTGGATCTGCTGCCCAAACTAACTCCATGGTGACCCTGGGATGCCTG | |
| | GTCAAGGGCTATTTCCCTGAGCCAGTGACAGTGACCTGGAACTCTGGATCCCTGTCCAGCGGTGTGCACACC | |
| | TTCCCAGCTGTCCTGCAGTCTGACCTCTACACTCTGAGCAGCTCAGTGACTGTCCCCTCCAGCACCTGGCCC | |
| | AGCGAGACCGTCACCTGCAACGTTGCCCACCCGGCCAGCAGCACCAAGGTGGACAAGAAAATTGTGCCCAGG | |
| | GATTGTGGTTGTAAGCCTTGCATATGTACAGTCCCAGAAGTATCATCTGTCTTCATCTTCCCCCCAAAGCCC | |
| | AAGGATGTGCTCACCATTACTCTGACTCCTAAGGTCACGTGTGTTGTGGTAGACATCAGCAAGGATGATCCC | |
| | GAGGTCCAGTTCAGCTGGTTTGTAGATGATGTGGAGGTGCACACAGCTCAGACGCAACCCCGGGAGGAGCAG | |
| | TTCAACAGCACTTTCCGCTCAGTCAGTGAACTTCCCATCATGCACCAGGACTGGCTCAATGGCAAGGAGTTC | |
| | AAATGCAGGGTCAACAGTGCAGCTTTCCCTGCCCCCATCGAGAAAACCATCTCCAAAACCAAAGGCAGACCG | |
| | AAGGCTCCACAGGTGTACACCATTCCACCTCCCAAGGAGCAGATGGCCAAGGATAAAGTCAGTCTGACCTGC | |
| | ATGATAACAGACTTCTTCCCTGAAGACATTACTGTGGAGTGGCAGTGGAATGGGCAGCCAGCGGAGAACTAC | |
| | AAGAACACTCAGCCCATCATGGACACAGATGGCTCTTACTTCATCTACAGCAAGCTCAATGTGCAGAAGAGC | |
| | AACTGGGAGGCAGGAAATACTTTCACCTGCTCTGTGTTACATGAGGGCCTGCACAACCACCATACTGAGAAG | |
| | AGCCTCTCCCACTCTCCTGGTAAATGA | |
| SEQ ID NO: 35 | ATGAAGTTGCCTGTTAGGCTGTTGGTGCTGATGGTCTGGATTCCTGCTTCCAGCAGTGATGTTTTGATGACC | Nucleic acid sequence encoding the complete |
| | CAAACTCCACTCTCCCTGCCTGTCAGTCTTGGAGATCAAGCCTCCATCTCTTGCAGATCTAGTCAGAGCATT | sequence of the light chain of 1D3 as defined by |
| | GTACATAGTAATGGAAACACCTATTTAGACTGGTACCTTCAGAAACCAGGCCAGTCTCCAAAGCTCCTGATC | SEQ ID NO: 21. The nucleic sequence that is |
| | TACAAAGTTTCCAACCGATTTTCTGGGGTCCCAGACAGGTTCAGTGGCAGTGGATCAGGGACAGATTTCACA | underlined encodes the leader sequence, which |
| | CTCAAAATCAGCAGAGTGGAGGCTGAGGATCTGGGAGTTTATTACTGCTTTCAAGGTTCACATGTTCCCACG | is removed in the mature expressed protein. |
| | TGGACGTTCGGTGGAGGCACCAAGCTGGAAATCAAACGGGCTGATGCTGCACCAACTGTATCCATCTTCCCA | |
| | CCATCCAGTGAGCAGTTAACATCTGGAGGTGCCTCAGTCGTGTGCTTCTTGAACAACTTCTACCCCAAAGAC | |
| | ATCAATGTCAAGTGGAAGATTGATGGCAGTGAACGACAAAATGGCGTCCTGAACAGTTGGACTGATCAGGAC | |
| | AGCAAAGACAGCACCTACAGCATGAGCAGCACCCTCACGTTGACCAAGGACGAGTATGAACGACATAACAGC | |
| | TATACCTGTGAGGCCACTCACAAGACATCAACTTCACCCATTGTCAAGAGCTTCAACAGGAATGAGTGTTAG | |
| SEQ ID NO: 36 | MTPTTTTAELTTEFDYDEAATPCVFTDVLNQSKPVTLFLYGVVFLFGSIGNFLVIFTITWRRRIQCSGDVYF | The full sequence of US28 as encoded by HCMV |
| | INLAAADLLFVCTLPLWMQYLLDANSLASVPCTLLTACFYVAMFASLCFITEIALDRYYAIVYMRYRPVKQA | strain Toledo (Accession number GU937742). |
| | CLFSIFWWIFAVIIAIPHFMVVTKKNNQCMTDYDYLEVSYPIILNVELMLGAFVIPLSVISYCYYRISRIVA | The sequence of ECD3 is underlined. |
| | VSQSRHKGRIVRVLIAVVLVFIIFWLPYHLTLFVDTLKLLKWISSSCEFERSLKRALILTESLAFCHCCLNP | |
| | LLYVFVGTKFRQELHCLLAEFRQRLFSRDVSWYHSMSFSRRSSPSRRETSSDTLSDEVCRVSQIIP | |
| SEQ ID NO: 37 | MTPTTTTAELTTEFDYDEDATPCVFTDVLNQSKPVTLFLYGVVFLFGSIGNFLVIFTITWRRRIQCSGDVYF | The full sequence of US28 as encoded by HCMV |
| | INLAAADLLFVCTLPLWMQYLLDANSLASVPCTLLTACFYVAMFASLCFITEIALDRYYAIVYMRYRPVKQA | strain Towne (Accession number FJ616285). |
| | CLFSIFWWIFAVIIAIPHFMVVTKKDNQCMTDYDYLEVSYPIILNVELMLGAFVIPLSVISYCYYRISRIVA | The sequence of ECD3 is underlined. |
| | VSQSRHKGRIVRVLIAVVLVFIIFWLPYHLTLFVDTLKLLKWISSSCEFERSLKRALILTESLAFCHCCLNP | |
| | LLYVFVGTKFRQELHCLLAEFRQRLFSRDVSWYHSMSFSRRSSPSRRETSSDTLSDEVCRVSQIIP | |
| SEQ ID NO: 38 | MTPTTTTAELTTEFDYDEDATPCVFTDVLNQSKPVTLFLYGVVFLFGSIGNFLVIFTITWRRRIQCSGDVYF | The full sequence of US28 as encoded by HCMV |
| | INLAAADLLFVCTLPLWMQYLLDANSLASVPCTLLTACFYVAMFASLCFITEIALDRYYAIVYMRYRPVKQA | strain VR1814 (Accession number GU179289). |
| | CLFSIFWWIFAVIIAIPHFMVVTKKDNQCMTDYDYLEVSYPIILNVELMLGAFVIPLSVISYCYYRISRIVA | The sequence of ECD3 is underlined. |
| | VSQSRAKGRIVRVLTAVVLVFIIFWLPYHLTLFVDTLKLLKWISSSCEFERSLKRALILTESLAFCHCCLNP | |
| | LLYVFVGTKFRQELHCLLAEFRQRLFSRDVSWYHSMSFSRRGSPSRRETSSDTLSDEVCRVSQIIP | |
| SEQ ID NO: 39 | MTPTTTTTELTTEFEYDLGATPCTFTDVLNQSKPVTLFLYGVVFLFGSVGNFLVIFTITWRRRIQCSGDVYF | The full sequence of US28 as encoded by HCMV |
| | INLAAADLLFVCTLPLWMQYLLDANSLASVPCTLLTACFYVAMFASLCFITEIALDRYYAIVYMRYRPVKQA | strain TB40/E(Accession number KF297339). |
| | CLFSIFWWIFAVIIAIPHFMVVTKKDNQCMTDYDYLEVSYPIILNVELMLGAFVIPLSVISYCYYRISRIVA | The sequence of ECD3 is underlined. |
| | VSQSRHKGRIVRVLIAVVLVFIIFWLPYHLTLFVDTLKLLKWISSSCEFERSLKRALILTESLAFCHCCLNP | |
| | LLYVFVGTKFRQELHCLLAEFRQRLFSRDVSWYHSMSFSRRSSPSRRETSSDTLSDEVCRVSQIIP | |
| SEQ ID NO: 40 | MTPTTTTAELTIEFDYDEDATPCVFTDVLNQSKPVTLFLYGVVFLFGSIGNFLVIFTITWRRRIQCSGDVYF | The full sequence of US28 as encoded by HCMV |
| | INLAAADLLFVCTLPLWMQYLLDANSLASVPCTLLTACFYVAMFASLCFITETALDRYYAIVYMRYRPVKQA | strain Merlin (Accession number AY446894). The |
| | CLFSIFWWIFAVIIAIPHFMVVTKKDNQCMTDYDYLEVSYPIILNVELMIGAFVIPLSVISYCYYRISRIVA | sequence of ECD3 is underlined. |
| | VSQSRHKGRIVRVLIAVVLVFIIFWLPYHLTLFVDTLKLLKWISSSCEFERSLKRALILTESLAFCHCCLNP | |
| | LLYVFVGTKFRQELHCLLAEFRQRLFSRDVSWYHSMSFSRRGSPSRRETSSDTLSDEVCRVSQIIP | |
| SEQ ID NO: 41 | MTPTTTTAELTTEFDYDEAATPCVFTDVLNQSKPVTLFLYGVVFLFGSIGNFLVIFTITWRRRIQCSGDVYF | The full sequence of US28 as encoded by HCMV |
| | INLAAADLLFVCTLPLWMQYLLDANSLASVPCTLLTACFYVAMFASLCFITEIALDRYYAIVYMRYRPVKQA | strain JP (Accession number GQ221975). The |
| | CLFSIFWWIFAVIIAIPHFMVVTKKDNQCMTDYDYLEVSYPIILNVELMIGAFVIPLSVISYCYYRISRIVA | sequence of ECD3 is underlined. |
| | VSQSRHKGRIVRVLIAVVLVFIIFWLPYHLTLFVDTLKLLKWISSSCEFERSLKRALILTESLAFCHCCLNP | |
| | LLYVFVGTKFRQELHCLLAEFRQRLFSRDVSWYHSMSFSRRSSPSRRETSSDTLSDEVCRVSQIIP | |
| SEQ ID NO: 42 | MTPTTTTAELTTEFDYDEDATPCVFTDVLNQSKPVTLFLYGVVFLFGSIGNFLVIFTITWRRRIQCSGDVYF | The full sequence of US28 as encoded by HCMV |
| | INLAAADLLFVCTLPLWMQYLLDANSLASVPCTLLTACFYVAMFASLCFITEIALDRYYAIVYMRYRPVKQA | strain Ad169 (Accession number X17403.1). The |
| | CLFSIFWWIFAVIIAIPHFMVVTKKDNQCMTDYDYLEVSYPIILNVELMLGAFVIPLSVISYCYYRISRIVA | sequence of ECD3 is underlined. |
| | VSQSRHKGRIVRVLIAVVLVFIIFWLPYHLTLFVDTLKLLKWISSSCEFERSLKRALILTESLAFCHCCLNP | |
| | LLYVFVGTKFRQELHCLLAEFRQRLFSRDVSWYHSMSFSRRSSPSRRETSSDTLSDEVCRVSQIIP | |
| SEQ ID NO: 43 | MTPPTTTAELTTEFDYDEAATPCVFTDVLNQSKPVTLFLYGVVFLFGSIGNFLVIFTITWRRRIQCSGDVYF | The full sequence of US28 as encoded by HCMV |
| | INLAAADLLFVCTLPLWMQYLLDHNSLASVPCTLLTACFYVAMFASLCFITEIALDRYYAIVYMRYRPVKQA | strain AF1 (Accession number GU179291.1). |
| | CLFSIFWWIFAVIIAIPHFMVVTKKDNQCMTDYDYLEVSYPIILNVELMLGAFVIPLSVISYCYYRISRIVA | The sequence of ECD3 is underlined. |
| | VSQSRHKGRIVRVLIAVVLVFIIFWLPYHLTLFVDTLKLLKWISSSCEFERSLKRALILTESLAFCHCCLNP | |
| | LLYVFVGTKFRQELHCLLAEFRQRLFSRDVSWYHSMSFSRRSSPSRRETSSDTLSDEVCRVSQIIP | |
| SEQ ID NO: 44 | MTPTTTTAELTTEFDYDDEATPCVLTDVLNQSKPVTLFLYGVVFLFGSIGNFLVIFTITWRRRIQCSGDVYF | The full sequence of US28 as encoded by HCMV |
| | INLAAADLLFVCTLPLWMQYLLDANSLASVPCTLLTACFYVAMFASLCFITEIALDRYYAIVYMRYRPVKQA | strain VHL/E (Accession number L20501.1). The |
| | CLFSIFWWIFAVIIAIPHFMVVTKKDNQCMTDYDYLEVSYPIILNVELMLGAFVIPLSVISYCYYRISRIVA | sequence of ECD3 is underlined. |
| | VSQSRHKGRIVRVLIAVVLVFIIFWLPYHLTLFVDTLKLLKWISSSCEFEKSLKRALILTESLAFCHCCLNP | |
| | LLYVFVGTKFRQELHCLLAEFRQRLFSRDVSWYHSMSFSRRSSPSRRETSSDTLSDEACRVSQIIP | |
| SEQ ID NO: 45 | MTPTTTTAELTTEFDYDEDATPCVFTDVLNQSKPVTLFLYGVVFLFGSIGNFLVIFTITWRRRIQCSGDVYF | The full sequence of US28 as encoded by HCMV |
| | INLAAADLLFVCTLPLWMQYLLDANSLASVPCTLLTACFYVAMFASLCFITEIALDRYYAIVYMRYRPVKQA | strain DAVIS (Accession number JX512198.1). |
| | CLFSIFWWIFAVIIAIPHFMVVTKKDNQCMTDYDYLEVSYPIILNTELMLGAFVIPLSVISYCYYRISRIVA | The sequence of ECD3 is underlined. |
| | VSQSRHKGRIVRVLIAVVLVFIIFWLPYHLTLFVDTLKLLKWISSSCEFEKSLKRALILTESLAFCHCCLNP | |
| | LLYVFVGTKFRQELHCLLAEFRQRLFSRDVSWYHSMSFSRRSSPSRRETSSDTLSDEACRVSQIIP | |
| SEQ ID NO: 46 | MTPTTTTAELTTEFDYDEAATPCVFTDVLNQSKPVTLFLYGVVFIFGSIGNFLVIFTITWRRRIQCSGDVYF | The full sequence of US28 as encoded by HCMV |
| | INLAAADLLFVCTLPLWMQYLLDANSLASVPCTLLTACFYVAMFASLCFITEIALDRYYAIVYMRYRPVKQA | strain TR (Accession number KF021605.1). The |
| | CLFSIFWWIFAVIIAIPHFMVVTKKNNQCMTDYDYLEVSYPIILNVELMLGAFVIPLSVISYCYYRISRIVA | sequence of ECD3 is underlined. |
| | VSQSRHKGRIVRVLIAVVLVFIIFWLPYHLTLFVDTLKLLKWISSSCEFEKSLKRALILTESLAFCHCCLNP | |
| | LLYVFVGTKFRQELHCLLAEFRQRLFSRDVSWYHSMSFSRRSSPSRRETSSDTLSDEVCRVSQIIP | |
| SEQ ID NO: 47 | MTPTTTTAELTTEFDYDEAATPCVFTDVLNQSKPVTLFLYGVVFLFGSIGNFLVIFTITWRRRIQCSGDVYF | US28 fusion protein, comprising US28 sequence |
| | INLAAADLLFVCTLPLWMQYLLDANSLASVPCTLLTACFYVAMFASLCFITETALDRYYAIVYMRYRPVKQA | from HCMV DB strain (Acc. Number: KT959235), |
| | CLFSIFWWIFAVITAIPHFMVVIKKNNQCMTDYDYLEVSYPIILNVELMIGAFVIPLSVISYCYYRISRIVA | fused to a 6His tag at its C terminal part |
| | VSQSRHKGRIVRVLIAVVLVFIIFWLPYHLTLFVDTLKLLKWISSSCEFERSLKRALILTESLAFCHCCLNP | |
| | LLYVFVGTKFRQELHCLLAEFRQRLFSRDVSWYHSMSFSRRSSPSRRETSSDTLSDEVCRVSQIIPAAALEG | |
| | SHHHHHH | |
| SEQ ID NO: 48 | QVQLQQSGAELARPGASVKMSCKASGYTFTRYTMHWVKQRPGQGLEWIGYINPSRGYTNYNQKEKDKATLTT | Mouse OKT3 (mOKT3) heavy chain variable |
| | DKSSSTAYMQLSSLTSEDSAVYYCARYYDDHYCLDYWGQGTTLTVSS | region |
| SEQ ID NO: 49 | RYTMH | OKT3 V_{H}-CDR1 sequence |
| SEQ ID NO: 50 | YINPSRGYTNYNQKFK | OKT3 V_{H}-CDR2 sequence |
| SEQ ID NO: 51 | YCARYYDD | OKT3 V_{H}-CDR3 sequence |
| SEQ ID NO: 52 | QIVLTQSPAIMSASPGEKVTMTCSASSSVSYMNWYQQKSGTSPKRWIYDTSKLASGVPAHFRGSGSGTSYSL | mOKT3 light chain variable region |
| | TISGMEAEDAATYYCQQWSSNPFTFGSGTKLEIN | |
| SEQ ID NO: 53 | SASSSVSYMNW | OKT3 V_{L}-CDR1 sequence |
| SEQ ID NO: 54 | TSKLASG | OKT3 V_{L}-CDR2 sequence |
| SEQ ID NO: 55 | QWSSNPFTF | OKT3 VL-CDR3 sequence |
| SEQ ID NO: 56 | GGGGSGGGGSGGGGS | Linker sequence |
| SEQ ID NO: 57 | GGGGS | Partial linker sequence |
| SEQ ID NO: 58 | [EVKLVESGGGLVQPGGSLRLACATSGFTFTDYYMSWVRQPPGKALEWLGFIRSKANGYTTEYSASVKGRFT | A first polypeptide sequence of a preferred BITE |
| | ISRDNSQSILYLQMNTLRSEDSATYYCARDERRTAWLAYWGQGTLVTVSA][ASTKGPSVFPLAPSSKSTSG | molecule comprising a heavy chain sequence that |
| | GTAALGCLVKDYFPEPVTVSWNSGALTSGVHTFPAVLQSSGLYSLSSVVTVPSSSLGTQTYICNVNHKPSNT | includes the VH region of 1D3, a linker sequence, |
| | KVDKKVEPKSCDKTHTCPPCPAPELLGGP][SVFLFPPKPKDTIMISRTPEVTCVVVDVSHEDPEVKFNWYV | and the sequence of an mOKT3 scFv |
| | DGVEVHNAKTKPREEQYNSTYRVVSVLTVLHQDWINGKEYKCKVSNKALPAPIEKTISKAKGQPREPQVYTL | For additional information, the start and end of |
| | PPSRDELT][KNQVSLTCLVKGFYPSDIAVEWESNGQPENNYKTTPPVLDSDGSFFLYSKLTVDKSRWQQGN | separate functional blocks of sequence are |
| | VFSCSVMHEALHNHYTQKSLSLSPGK][GGGGSGGGGS][QVQLQQSGAELARPGASVKMSCKASGYTFTRY | indicated by “[“ and ”]”, respectively, although it is |
| | TMHWVKQRPGQGLEWIGYINPSRGYTNYNQKFKDKATLITDKSSSTAYMQLSSLTSEDSAVYYCARYYDDHY | to be understood that the first polypeptide |
| | CLDYWGQGTTLTVSS][GGGGSGGGGSGGGGS][QIVLTQSPAIMSASPGEKVTMTCSASSSVSYMNWYQQK | contains all of these sequences in a single |
| | SGTSPKRWIYDTSKLASGVPAHFRGSGSGTSYSLTISGMEAEDAATYYCQQWSSNPFTFGSGTKLEIN] | continuous polypeptide chain, in the order |
| | | presented. |
| SEQ ID NO: 59 | [DVLMTQTPLSLPVSLGDQASISCRSSQSIVHSNGNTYLDWYLQKPGQSPKLLIYKVSNRFSGVPDRFSGSG | A second polypeptide sequence of a preferred |
| | SGTDFTLKISRVEAEDLGVYYCFQGSHVPTWTFGGGTKLEIK][RTVAAPSVFIFPPSDEQLKSGTASVVCL | BITEmolecule comprising a light chain sequence |
| | LNNFYPREAKVQWKVDNALQSGNSQESVTEQDSKDSTYSLSSTLTLSKADYEKHKVYACEVTHQGLSSPVTK | that includes the VLregion of 1D3, or example |
| | SFNRGEC] | having the sequence of SQE ID NO: 59 |
| | | For additional information, the start and end of |
| | | separate functional blocks of sequence are |
| | | indicated by “[“ and ”]”, respectively, although it is |
| | | to be understood that the first polypeptide |
| | | contains all of these sequences in a single |
| | | continuous polypeptide chain, |
| SEQ ID NO: 60 | EVQLVESGGGLVQPGGSLRLACAVSGPGLIFKFTGVAWYRRQVPGAKRGLVALITGDGATRYGDSVKGRFTV | Core sequence of VUN100, as shown on page 24 |
| | SRDIAAKRVYLEMNDLRSEDTAVYYCKTGEYWGQGTQVTVSS | of WO 2019/151865 |
| SEQ ID NO: 61 | MHSSALLCCLVLLTGVRAEVQLVESGGGLVQPGGSLRLACAVSGPGLIFKFTGVAWYRRQVPGAKRGLVALI | VUN100 sequence of SEQ ID NO: 60, with |
| | TGDGATRYGDSVKGRFTVSRDIAAKRVYLEMNDLRSEDTAVYYCKTGEYWGQGTQVTVSSEQKLISEEDLHH | additional N-terminal signal peptide (underlined), |
| | HHHH | and C-terminal Myc-6His tag sequences (double |
| | | underlined) |
| SEQ ID NO: 62 | EVQLVESGGGLVQPGGSLRLACAVSGPGLIFKFTGVAWYRRQVPGAKRGLVALITGDGATRYGDSVKGRFTV | VUN100b, a bivalent VUN100 molecule, |
| | SRDIAAKRVYLEMNDLRSEDTAVYYCKTGEYWGQGTQVTVSSGGGGSGGGGSGGGGSGGGGSGGGGSGGGGS | consisting of two copies of VUN100, joined by a |
| | EVQLVESGGGLVQPGGSLRLACAVSGPGLIFKFTGVAWYRRQVPGAKRGLVALITGDGATRYGDSVKGRETV | linker sequence (linker sequence underlined) |
| | SRDIAAKRVYLEMNDLRSEDTAVYYCKTGEYWGQGTQVTVSS | |
| SEQ ID NO: 63 | MHSSALLCCLVLLTGVRAEVQLVESGGGLVQPGGSLRLACAVSGPGLIFKFTGVAWYRRQVPGAKRGLVALI | VUN100b sequence of SEQ ID NO: 62, with |
| | TGDGATRYGDSVKGRFTVSRDIAAKRVYLEMNDLRSEDTAVYYCKTGEYWGQGTQVTVSSGGGGSGGGGSGG | additional N-terminal signal peptide (underlined), |
| | GGSGGGGSGGGGSGGGGSEVQLVESGGGLVQPGGSLRLACAVSGPGLIFKFTGVAWYRRQVPGAKRGLVALI | and C-terminal Myc-6His tag sequences (double |
| | TGDGATRYGDSVKGRFTVSRDIAAKRVYLEMNDLRSEDTAVYYCKTGEYWGQGTQVTVSSEQKLISEEDLHH | underlined) |
| | HHHH | |
| SEQ ID NO: 65 | ATGAAGTTGTGGCTGAACTGGATTTTCCTTGTAACACTTTTAAATGGTATCCAGTGTGAAGTGAAGCTGGTG | Nucleic acid encoding the immature form of the |
| | GAGTCTGGGGGAGGCTTAGTGAAGCCTGGAGGGTCCCTGAAACTCTCCTGTGCAGCCTCTGGATTCACTTTC | recombinantly expressed VH sequence of 1G9 as |
| | AGTAGCTATGCCATGTCTTGGGTTCGCCAGACTCCAGAGAAGAGGCTGGAGTGGGTCGCATCCATTAGTAGT | defined by SEQ ID NO: 67 |
| | GGTGGTAGCACCTACTATCCAGACAGTGTGAAGGGCCGATTCACCATCTCCAGAGATAATGCCAGGAACATC | |
| | CTGTACCTGCAAATGAGCAGTCTGAGGTCTGAGGACACGGCCATGTATTACTGTGCAAGAGGCGGGTCTACT | |
| | ATGATTACGACGGGGCTGGGGTTTGCTTACTGGGGCCAAGGGACTCTGGTCACTGTCTCTGCA | |
| SEQ ID NO: 66 | GAAGTGAAGCTGGTGGAGTCTGGGGGAGGCTTAGTGAAGCCTGGAGGGTCCCTGAAACTCTCCTGTGCAGCC | Nucleic acid encoding the mature form of VH |
| | TCTGGATTCACTTTCAGTAGCTATGCCATGTCTTGGGTTCGCCAGACTCCAGAGAAGAGGCTGGAGTGGGTC | sequence of 1G9 as defined by SEQ ID NO: 68 |
| | GCATCCATTAGTAGTGGTGGTAGCACCTACTATCCAGACAGTGTGAAGGGCCGATTCACCATCTCCAGAGAT | |
| | AATGCCAGGAACATCCTGTACCTGCAAATGAGCAGTCTGAGGTCTGAGGACACGGCCATGTATTACTGTGCA | |
| | AGAGGCGGGTCTACTATGATTACGACGGGGCTGGGGTTTGCTTACTGGGGCCAAGGGACTCTGGTCACTGTC | |
| | TCTGCA | |
| SEQ ID NO: 67 | MKLWLNWIFLVTLLNGIQCEVKLVESGGGLVKPGGSLKLSCAASGFTFSSYAMSWVRQTPEKRLEWVASISS | Immature form of the recombinantly expressed |
| | GGSTYYPDSVKGRFTISRDNARNILYLQMSSLRSEDTAMYYCARGGSTMITTGLGFAYWGQGTLVTVSA | VH sequence of 1G9, wherein positions 1-19 |
| | | correspond to a leader sequence |
| SEQ ID NO: 68 | EVKLVESGGGLVKPGGSIKLSCAASGFTFSSYAMSWVRQTPEKRLEWVASISSGGSTYYPDSVKGRFTISRD | Mature form of VH sequence of 1G9, without the |
| | NARNILYLQMSSLRSEDTAMYYCARGGSTMITTGLGFAYWGQGTLVTVSA | leader sequence |
| SEQ ID NO: 69 | ATGAAGTTGCCTGTTAGGCTGTTGGTGCTGATGGTCTGGATTCCTGCTTCCAGCAGTCAAATTGTTCTCACC | Nucleic acid encoding the immature form of the |
| | CAGTCTCCAGCAATCATGTCTGCATCTCCAGGGGAGAAGGTCACCATGACCTGCAGTGCCAGCTCAAGTGTA | recombinantly expressed VL sequence of 1G9 as |
| | AGTTACATGCACTGGTACCAGCAGAAGTCAGGCACCTCCCCCAAAAGATGGATTTATGACACATCCAAACTG | defined by SEQ ID NO: 71 |
| | GCTTCTGGAGTCCCTGCTCGCTTCAGTGGCAGTGGGTCTGGGACCTCTTACTCTCTCACAATCAGCAGCATG | |
| | GAGGCTGAAGATGCTGCCACTTATTACTGCCAGCAGTGGAGTAGTAACCCACCCCTCACGTTCGGTGCTGGG | |
| | ACCAAGCTGGAGCTGAAA | |
| SEQ ID NO: 70 | CAAATTGTTCTCACCCAGTCTCCAGCAATCATGTCTGCATCTCCAGGGGAGAAGGTCACCATGACCTGCAGT | Nucleic acid encoding the mature form of VL |
| | GCCAGCTCAAGTGTAAGTTACATGCACTGGTACCAGCAGAAGTCAGGCACCTCCCCCAAAAGATGGATTTAT | sequence of 1G9 as defined by SEQ ID NO: 72 |
| | GACACATCCAAACTGGCTTCTGGAGTCCCTGCTCGCTTCAGTGGCAGTGGGTCTGGGACCTCTTACTCTCTC | |
| | ACAATCAGCAGCATGGAGGCTGAAGATGCTGCCACTTATTACTGCCAGCAGTGGAGTAGTAACCCACCCCTC | |
| | ACGTTCGGTGCTGGGACCAAGCTGGAGCTGAAA | |
| SEQ ID NO: 71 | MKLPVRLLVLMVWIPASSSQIVLTQSPAIMSASPGEKVTMTCSASSSVSYMHWYQQKSGTSPKRWIYDTSKL | Immature form of the recombinantly expressed |
| | ASGVPARFSGSGSGTSYSLTISSMEAEDAATYYCQQWSSNPPLTFGAGTKLELK | VL sequence of 1G9, wherein positions 1-19 |
| | | correspond to a leader sequence |
| SEQ ID NO: 72 | QIVLTQSPAIMSASPGEKVTMTCSASSSVSYMHWYQQKSGTSPKRWIYDTSKLASGVPARFSGSGSGTSYSL | Mature form of VL sequence of 1G9, without the |
| | TISSMEAEDAATYYCQQWSSNPPLTFGAGTKLELK | leader sequence |
| SEQ ID NO: 73 | AGCTATGCCATGTCT | Nucleic acid encoding the VH-CDR1 sequence of |
| | | 1G9, 1E8, as defined by SEQ ID NO: 76 |
| SEQ ID NO: 74 | TCCATTAGTAGTGGTGGTAGCACCTACTATCCAGACAGTGTGAAGGGC | Nucleic acid encoding the VH-CDR2 sequence of |
| | | 1G9 as defined by SEQ ID NO: 77 |
| SEQ ID NO: 75 | GGCGGGTCTACTATGATTACGACGGGGCTGGGGTTTGCTTAC | Nucleic acid encoding the VH-CDR3 sequence of |
| | | 1G9 as defined by SEQ ID NO: 78 |
| SEQ ID NO: 76 | SYAMS | VH-CDR1 sequence of 1G9, 1E8 |
| SEQ ID NO: 77 | SISSGGSTYYPDSVKG | VH-CDR2 sequence of 1G9 |
| SEQ ID NO: 78 | GGSTMITTGLGFAY | VH-CDR3 sequence of 1G9 |
| SEQ ID NO: 79 | AGTGCCAGCTCAAGTGTAAGTTACATGCAC | Nucleic acid encoding the VL-CDR1 sequence of |
| | | 1G9, 1E8, as defined by SEQ ID NO: 82 |
| SEQ ID NO: 80 | GACACATCCAAACTGGCTTCT | Nucleic acid encoding the VL-CDR2 sequence of |
| | | 1G9, 1C10, 1A10, as defined by SEQ ID NO: 83 |
| SEQ ID NO: 81 | CAGCAGTGGAGTAGTAACCCACCCCTCACG | Nucleic acid encoding the VL-CDR3 sequence of |
| | | 1 G9 as defined by SEQ ID NO: 84 |
| SEQ ID NO: 82 | SASSSVSYMH | VL-CDR1 sequence of 1G9, 1E8 |
| SEQ ID NO: 83 | DTSKLAS | VL-CDR2 sequence of 1G9, 1C10, 1A10 |
| SEQ ID NO: 84 | QQWSSNPPLT | VL-CDR3 sequence of 1G9 |
| SEQ ID NO: 85 | ATGAAGTTGTGGCTGAACTGGATTTTCCTTGTAACACTTTTAAATGGTATCCAGTGTGAAGTGAAGCTGGTG | Nucleic acid encoding the immature form of the |
| | GAGTCTGGGGGAGACTTAGTGAAGCCTGGAGGGTCCCTGAAACTCTCCTGTGCAGCCTCTGGATTCACTTTC | recombinantly expressed VH sequence of 1E8 as |
| | AGTAGCTATGCCATGTCTTGGGTTCGCCAGACTCCAGAGAAGAGGCTGGAGTGGGTCGCATCCATTAGTAGT | defined by SEQ ID NO: 87 |
| | GGTGGTAGAACCTACTATCCAGACAGTGTGAAGGGCCGATTCACCATCTCCAGAGATAATGCCAGGAACATC | |
| | CTGTACCTGCAAATGAGCAGTCTGAGGTCTGAAGACACGGCCATCTATTACTGTGCACGAGGCGGGACTCGT | |
| | CATTCCTACGGCAACGGGTTTGACTACTGGGGCCAAGGCACCACTCTCACAGTCTCCTCA | |
| SEQ ID NO: 86 | GAAGTGAAGCTGGTGGAGTCTGGGGGAGACTTAGTGAAGCCTGGAGGGTCCCTGAAACTCTCCTGTGCAGCC | Nucleic acid encoding the mature form of VH |
| | TCTGGATTCACTTTCAGTAGCTATGCCATGTCTTGGGTTCGCCAGACTCCAGAGAAGAGGCTGGAGTGGGTC | sequence of 1E8 as defined by SEQ ID NO: 88 |
| | GCATCCATTAGTAGTGGTGGTAGAACCTACTATCCAGACAGTGTGAAGGGCCGATTCACCATCTCCAGAGAT | |
| | AATGCCAGGAACATCCTGTACCTGCAAATGAGCAGTCTGAGGTCTGAAGACACGGCCATCTATTACTGTGCA | |
| | CGAGGCGGGACTCGTCATTCCTACGGCAACGGGTTTGACTACTGGGGCCAAGGCACCACTCTCACAGTCTCC | |
| | TCA | |
| SEQ ID NO: 87 | MKLWLNWIFLVTLLNGIQCEVKLVESGGDLVKPGGSLKLSCAASGFTFSSYAMSWVRQTPEKRLEWVASISS | Immature form of the recombinantly expressed |
| | GGRTYYPDSVKGRFTISRDNARNILYLQMSSLRSEDTATYYCARGGTRHSYGNGFDYWGQGTILTVSS | VH sequence of 1E8, wherein positions 1-19 |
| | | correspond to a leader sequence |
| SEQ ID NO: 88 | EVKLVESGGDLVKPGGSLKLSCAAVLSGFTFSSYAMSWVRQTPEKRLEWVASISSGGRTYYPDSVKGRFTISRD | Mature form of VH sequence of 1E8, without the |
| | NARNILYLQMSSLRSEDTAIYYCARGGTRHSYGNGFDYWGQGTTLTVSS | leader sequence |
| SEQ ID NO: 89 | ATGAAGTTGCCTGTTAGGCTGTTGGTGCTGATGGTCTGGATTCCTGCTTCCAGCAGTCAAATTGTTCTCTCC | Nucleic acid encoding the immature form of the |
| | CAGTCTCCAACAATCATGTCTGCATCTCCAGGGGAGAAGGTCACCATGACCTGCAGTGCCAGCTCAAGTGTA | recombinantly expressed VL sequence of 1E8 as |
| | AGTTACATGCACTGGTACCAGCAGAAGTCAGGCACCTCCCCCAAAAGATGGATTTATGACTCATCCAAACTG | defined by SEQ ID NO: 91 |
| | GCTTCTGGAGTCCCTGCTCGCTTCAGTGGCAGTGGGTCTGGGACCTCTTACTCTCTCACAATCAGCAGCATG | |
| | GAGGCTGAAGATGCTGCCACTTATTACTGCCAGCAGTGGACTAGTAACCCACCCATCACGTTCGGTGCTGGG | |
| | ACCAAGCTGGAGCTGAAA | |
| SEQ ID NO: 90 | CAAATTGTTCTCTCCCAGTCTCCAACAATCATGTCTGCATCTCCAGGGGAGAAGGTCACCATGACCTGCAGT | Nucleic acid encoding the mature form of VL |
| | GCCAGCTCAAGTGTAAGTTACATGCACTGGTACCAGCAGAAGTCAGGCACCTCCCCCAAAAGATGGATTTAT | sequence of 1E8 as defined by SEQ ID NO: 92 |
| | GACTCATCCAAACTGGCTTCTGGAGTCCCTGCTCGCTTCAGTGGCAGTGGGTCTGGGACCTCTTACTCTCTC | |
| | ACAATCAGCAGCATGGAGGCTGAAGATGCTGCCACTTATTACTGCCAGCAGTGGACTAGTAACCCACCCATC | |
| | ACGTTCGGTGCTGGGACCAAGCTGGAGCTGAAA | |
| SEQ ID NO: 91 | MKLPVRLLVLMVWIPASSSQIVLSQSPTIMSASPGEKVTMTCSASSSVSYMHWYQQKSGTSPKRWIYDSSKL | Immature form of the recombinantly expressed |
| | ASGVPARFSGSGSGISYSLTISSMEAEDAATYYCQQWTSNPPITFGAGTKLELK | VL sequence of 1E8, wherein positions 1-19 |
| | | correspond to a leader sequence |
| SEQ ID NO: 92 | QIVLSQSPTIMSASPGEKVTMTCSASSSVSYMHWYQQKSGTSPKRWIYDSSKLASGVPARFSGSGSGTSYSL | Mature form of VL sequence of 1E8, without the |
| | TISSMEAEDAATYYCQQWTSNPPITFGAGTKLELK | leader sequence |
| SEQ ID NO: 93 | TCCATTAGTAGTGGTGGTAGAACCTACTATCCAGACAGTGTGAAGGGC | Nucleic acid encoding the VH-CDR2 sequence of |
| | | 1E8 as defined by SEQ ID NO: 95 |
| SEQ ID NO: 94 | GGCGGGACTCGTCATTCCTACGGCAACGGGTTTGACTAC | Nucleic acid encoding the VH-CDR3 sequence of |
| | | 1E8 as defined by SEQ ID NO: 96 |
| SEQ ID NO: 95 | SISSGGRTYYPDSVKG | VH-CDR2 sequence of 1E8 |
| SEQ ID NO: 96 | GGTRHSYGNGFDY | VH-CDR3 sequence of 1E8 |
| SEQ ID NO: 97 | GACTCATCCAAACTGGCTTCT | Nucleic acid encoding the VL-CDR2 sequence of |
| | | 1E8 as defined by SEQ ID NO: 99 |
| SEQ ID NO: 98 | CAGCAGTGGACTAGTAACCCACCCATCACG | Nucleic acid encoding the VL-CDR3 sequence of |
| | | 1E8 as defined by SEQ ID NO: 100 |
| SEQ ID NO: 99 | DSSKLAS | VL-CDR2 sequence of 1E8 |
| SEQ ID NO: 100 | QQWTSNPPIT | VL-CDR3 sequence of 1E8 |
| SEQ ID NO: 101 | ATGAAGTTGTGGCTGAACTGGATTTTCCTTGTAACACTTTTAAATGGTATCCAGTGTGAAGTGAAACTGGTT | Nucleic acid encoding the immature form of the |
| | GAGTCTGGGGGAGTCTTAGTGAAGCCTGGAGGGTCCCTGAAATTTTCCTGTGCAGCCTCTGGATTCACTTTC | recombinantly expressed VH sequence of 1C10 |
| | AGTAGCCATGCCTTGTCTTGGGTTCGCCAGACTCCAGAGAAGAGACTGGAGTGGGTCGCATCCATTAGTAGT | as defined by SEQ ID NO: 103 |
| | CGTGGTCGAACCTACTATCCAGACAGTGTAAAGGGCCGATTCACCGTCTCCAGAGATAATGCCAGGAACATC | |
| | CTGTATCTGCAAGTGAGCAGTCTGAGGTCTGAGGACACGGCCATGTATTATTGTACAAGAGGCGGGACTCAT | |
| | TATTCCTACGGCAACGGTTTTGACTTCTGGGGCCAAGGCACCACTCTCACAGTCTCCTCA | |
| SEQ ID NO: 102 | GAAGTGAAACTGGTTGAGTCTGGGGGAGTCTTAGTGAAGCCTGGAGGGTCCCTGAAATTTTCCTGTGCAGCC | Nucleic acid encoding the mature form of VH |
| | TCTGGATTCACTTTCAGTAGCCATGCCTTGTCTTGGGTTCGCCAGACTCCAGAGAAGAGACTGGAGTGGGTC | sequence of 1C10 as defined by SEQ ID NO: 104 |
| | GCATCCATTAGTAGTCGTGGTCGAACCTACTATCCAGACAGTGTAAAGGGCCGATTCACCGTCTCCAGAGAT | |
| | AATGCCAGGAACATCCTGTATCTGCAAGTGAGCAGTCTGAGGTCTGAGGACACGGCCATGTATTATTGTACA | |
| | AGAGGGGGGACTCATTATTCCTACGGCAACGGTTTTGACTTCTGGGGCCAAGGCACCACTCTCACAGTCTCC | |
| | TCA | |
| SEQ ID NO: 103 | MKLWLNWIFLVTLLNGIQCEVKLVESGGVLVKPGGSLKFSCAASGFTFSSHALSWVRQTPEKRLEWVASISS | Immature form of the recombinantly expressed |
| | RGRTYYPDSVKGRFTVSRDNARNILYLQVSSLRSEDTAMYYCTRGGTHYSYGNGFDFWGQGTTLTVSS | VH sequence of 1C10, wherein positions 1-19 |
| | | correspond to a leader sequence |
| SEQ ID NO: 104 | EVKLVESGGVLVKPGGSLKESCAASGFTFSSHALSWVRQTPEKRLEWVASISSRGRTYYPDSVKGRFTVSRD | Mature form of VH sequence of 1C10, without the |
| | NARNILYLQVSSLRSEDTAMYYCTRGGTHYSYGNGEDEWGQGTTLTVSS | leader sequence |
| SEQ ID NO: 105 | ATGAAGTTGCCTGTTAGGCTGTTGGTGCTGATGGTCTGGATTCCTGCTTCCAGCAGTCAAATTGTTCTCACC | Nucleic acid encoding the immature form of the |
| | CAGTCTCCAGCAATCATGTCTGCATCTCCAGGGGAGAAGGTCACCATGACCTGCAGTGTCAGCTCAAGTGTT | recombinantly expressed VL sequence of 1C10 |
| | AGTTACATGCACTGGTACCAGCAGAAGTCAGGCACCTCCCCCAAAAGATGGATTTATGACACATCCAAACTG | as defined by SEQ ID NO: 107 |
| | GCTTCTGGAGTCCCTGCTCGCTTCAGTGGCAGTGGGTCTGGGACCTCTTACTCTCTCACAATCAGCACCATG | |
| | GAGGCTGAAGATGCTGCCACTTATTACTGCCAGCAGTGGAGTAATAACCCACCCATCACGTTCGGTGCTGGG | |
| | ACCAAGCTGGAACTGAAA | |
| SEQ ID NO: 106 | CAAATTGTTCTCACCCAGTCTCCAGCAATCATGTCTGCATCTCCAGGGGAGAAGGTCACCATGACCTGCAGT | Nucleic acid encoding the mature form of VL |
| | GTCAGCTCAAGTGTTAGTTACATGCACTGGTACCAGCAGAAGTCAGGCACCTCCCCCAAAAGATGGATTTAT | sequence of 1C10 as defined by SEQ ID NO: 108 |
| | GACACATCCAAACTGGCTTCTGGAGTCCCTGCTCGGTTCAGTGGCAGTGGGTCTGGGACCTCTTACTCTCTC | |
| | ACAATCAGCACCATGGAGGCTGAAGATGCTGCCACTTATTACTGCCAGCAGTGGAGTAATAACCCACCCATC | |
| | ACGTTCGGTGCTGGGACCAAGCTGGAACTGAAA | |
| SEQ ID NO: 107 | MKLPVRLLVLMVWIPASSSQIVLTQSPAIMSASPGEKVTMTCSVSSSVSYMHWYQQKSGTSPKRWIYDTSKL | Immature form of the recombinantly expressed |
| | ASGVPARFSGSGSGTSYSLTISTMEAEDAATYYCQQWSNNPPITFGAGTKLELK | VL sequence of 1C10, wherein positions 1-19 |
| | | correspond to a leader sequence |
| SEQ ID NO: 108 | QIVLTQSPAIMSASPGEKVTMTCSVSSSVSYMHWYQQKSGTSPKRWIYDTSKLASGVPARFSGSGSGTSYSL | Mature form of VL sequence of 1C10, without the |
| | TISTMEAEDAATYYCQQWSNNPPITFGAGTKLELK | leader sequence |
| SEQ ID NO: 109 | AGCCATGCCTTGTCT | Nucleic acid encoding the VH-CDR1 sequence of |
| | | 1C10, 1A10, as defined by SEQ ID NO: 112 |
| SEQ ID NO: 110 | TCCATTAGTAGTCGTGGTCGAACCTACTATCCAGACAGTGTAAAGGGC | Nucleic acid encoding the VH-CDR2 sequence of |
| | | 1C10, 1A10, as defined by SEQ ID NO: 113 |
| SEQ ID NO: 111 | GGCGGGACTCATTATTCCTACGGCAACGGTTTTGACTTC | Nucleic acid encoding the VH-CDR3 sequence of |
| | | 1C10, 1A10, as defined by SEQ ID NO: 114 |
| SEQ ID NO: 112 | SHALS | VH-CDR1 sequence of 1C10, 1A10 |
| SEQ ID NO: 113 | SISSRGRTYYPDSVKG | VH-CDR2 sequence of 1C10, 1A10 |
| SEQ ID NO: 114 | GGTHYSYGNGFDF | VH-CDR3 sequence of 1C10, 1A10 |
| SEQ ID NO: 115 | AGTGTCAGCTCAAGTGTTAGTTACATGCAC | Nucleic acid encoding the VL-CDR1 sequence of |
| | | 1C10, 1A10, as defined by SEQ ID NO: 117 |
| SEQ ID NO: 116 | CAGCAGTGGAGTAATAACCCACCCATCACG | Nucleic acid encoding the VL-CDR3 sequence of |
| | | 1C10, 1A10, as defined by SEQ ID NO: 118 |
| SEQ ID NO: 117 | SVSSSVSYMH | VL-CDR1 sequence of 1C10, 1A10 |
| SEQ ID NO: 118 | QQWSNNPPIT | VL-CDR3 sequence of 1C10, 1A10 |
| SEQ ID NO: 119 | ATGAAGTTGTGGCTGAACTGGATTTTCCTTGTAACACTTTTAAATGGTATCCAGTGTGAAGTGAAACTGGTT | Nucleic acid encoding the immature form of the |
| | GAGTCTGGGGGAGTCTTAGTGAAGCCTGGAGGGTCCCTGAAATTTTCCTGTGCAGCCTCTGGATTCACTCTC | recombinantly expressed VH sequence of 1A10 |
| | AGTAGCCATGCCTTGTCTTGGGTTCGCCAGACTCCAGAGAAGAGACTGGAGTGGGTCGCATCCATTAGTAGT | as defined by SEQ ID NO: 121 |
| | CGTGGTCGAACCTACTATCCAGACAGTGTAAAGGGCCGATTCACCGTCTCCAGAGATAATGCCAGGAACATC | |
| | CTGTATCTGCAAGTGAGCAGTCTGAGGTCTGAGGACACGGCCATGTATTATTGTACAAGAGGCGGGACTCAT | |
| | TATTCCTACGGCAACGGTTTTGACTTCTGGGGCCAAGGCACCACTCTCACAGTCTCCTCA | |
| SEQ ID NO: 120 | GAAGTGAAACTGGTTGAGTCTGGGGGAGTCTTAGTGAAGCCTGGAGGGTCCCTGAAATTTTCCTGTGCAGCC | Nucleic acid encoding the mature form of VH |
| | TCTGGATTCACTCTCAGTAGCCATGCCTTGTCTTGGGTTCGCCAGACTCCAGAGAAGAGACTGGAGTGGGTC | sequence of 1A10 as defined by SEQ ID NO: 122 |
| | GCATCCATTAGTAGTCGTGGTCGAACCTACTATCCAGACAGTGTAAAGGGCCGATTCACCGTCTCCAGAGAT | |
| | AATGCCAGGAACATCCTGTATCTGCAAGTGAGCAGTCTGAGGTCTGAGGACACGGCCATGTATTATTGTACA | |
| | AGAGGCGGGACTCATTATTCCTACGGCAACGGTTTTGACTTCTGGGGCCAAGGCACCACTCTCACAGTCTCC | |
| | TCA | |
| SEQ ID NO: 121 | MKLWLNWIFLVTLLNGIQCEVKLVESGGVLVKPGGSLKFSCAASGFTLSSHALSWVRQTPEKRLEWVASISS | Immature form of the recombinantly expressed |
| | RGRTYYPDSVKGRFTVSRDNARNILYLQVSSLRSEDTAMYYCTRGGTHYSYGNGFDFWGQGTTLTVSS | VH sequence of 1A10, wherein positions 1-19 |
| | | correspond to a leader sequence |
| SEQ ID NO: 122 | EVKLVESGGVLVKPGGSLKFSCAASGFTLSSHALSWVRQTPEKRLEWVASISSRGRTYYPDSVKGRFTVSRD | Mature form of VH sequence of 1A10, without the |
| | NARNILYLQVSSLRSEDTAMYYCTRGGTHYSYGNGFDFWGQGTTLTVSS | leader sequence |
| SEQ ID NO: 123 | ATGAAGTTGCCTGTTAGGCTGTTGGTGCTGATGGTCTGGATTCCTGCTTCCAGCAGTCAAATTGTTCTCACC | Nucleic acid encoding the immature form of the |
| | CAGTCTCCAGCAATCATGTCTGCATCTCCAGGGGAGAAGGTCACCATGACCTGCAGTGTCAGCTCAAGTGTT | recombinantly expressed VL sequence of 1A10 |
| | AGTTACATGCACTGGTACCAGCAGAAGTCAGGCACCTCCCCCAAAAGATGGATTTATGACACATCCAAACTG | as defined by SEQ ID NO: 125 |
| | GCTTCTGGAGTCCCTGCTCGCTTCAGTGGCAGTGGGTCTGGGACCTCTTACTCTCTCACAATCAGCACCATG | |
| | GAGGCTGAAGATGCTGCCACTTATTACTGCCAGCAGTGGAGTAATAACCCACCCATCACGTTCGGTGCTGGG | |
| | ACCAAGCTGGAACTGAAA | |
| SEQ ID NO: 124 | CAAATTGTTCTCACCCAGTCTCCAGCAATCATGTCTGCATCTCCAGGGGAGAAGGTCACCATGACCTGCAGT | Nucleic acid encoding the mature form of VL |
| | GTCAGCTCAAGTGTTAGTTACATGCACTGGTACCAGCAGAAGTCAGGCACCTCCCCCAAAAGATGGATTTAT | sequence of 1A10 as defined by SEQ ID NO: 126 |
| | GACACATCCAAACTGGCTTCTGGAGTCCCTGCTCGCTTCAGTGGCAGTGGGTCTGGGACCTCTTACTCTCTC | |
| | ACAATCAGCACCATGGAGGCTGAAGATGCTGCCACTTATTACTGCCAGCAGTGGAGTAATAACCCACCCATC | |
| | ACGTTCGGTGCTGGGACCAAGCTGGAACTGAAA | |
| SEQ ID NO: 125 | MKLPVRLLVLMVWIPASSSQIVLTQSPAIMSASPGEKVTMTCSVSSSVSYMHWYQQKSGTSPKRWIYDTSKL | Immature form of the recombinantly expressed |
| | ASGVPARFSGSGSGTSYSLTISTMEAEDAATYYCQQWSNNPPITFGAGTKLELK | VL sequence of 1A10, wherein positions 1-19 |
| | | correspond to a leader sequence |
| SEQ ID NO: 126 | QIVLTQSPAIMSASPGEKVTMTCSVSSSVSYMHWYQQKSGTSPKRWIYDTSKLASGVPARFSGSGSGTSYSL | Mature form of VL sequence of 1A10, without the |
| | TISTMEAEDAATYYCQQWSNNPPITFGAGTKLELK | leader sequence |
| SEQ ID NO: 127 | MTPTTTTAELTTEFDYDEDAAPCVLTDVLNQSKPVTLFLYGVVFLFGSIGNFLVIFTITWRRRIQCSGDVYF | The full sequence of US28 as encoded by HCMV |
| | INLAAADLLFVCTLPLWMQYLLDANSLASVPCTLLTACFYVAMFASLCFITEIALDRYYAIVYMRYRPVKQA | strain BL (Accession number MW980585.1). |
| | CLFSIFWWIFAVIIAIPHFMVVTKKDNQCMTDYDYLEVSYPIILNVELMLGAFVIPLSVISYCYYRISRIVA | The sequence of ECD3 is underlined. |
| | VSQSRHKGRIVRVLIAVVLVFIIFWLPYHLTLFLDTLKLLKWISSSCEFEKSLKRALILTESLAFCHCCLNP | |
| | LLYVFVGTKFRQELHCLLAEFRQRLFSRDVSWYHSMSFSRRSSPSRRETSSDTLSDEACRVSQIIP | |
| SEQ ID NO: 147 | ATGAAGTTGTGGCTGAACTGGATTTTCCTTGTAACACTTTTAAATGGTATCCAGTGTGAAGTGAAGCTGGTG | Nucleic acid sequence encoding the complete |
| | GAGTCTGGGGGAGGCTTAGTGAAGCCTGGAGGGTCCCTGAAACTCTCCTGTGCAGCCTCTGGATTCACTTTC | sequence of the heavy chain of 1G9, as defined |
| | AGTAGCTATGCCATGTCTTGGGTTCGCCAGACTCCAGAGAAGAGGCTGGAGTGGGTCGCATCCATTAGTAGT | by SEQ ID NO: 149. The nucleic sequence that |
| | GGTGGTAGCACCTACTATCCAGACAGTGTGAAGGGCCGATTCACCATCTCCAGAGATAATGCCAGGAACATC | is underlined encodes the leader sequence, |
| | CTGTACCTGCAAATGAGCAGTCTGAGGTCTGAGGACACGGCCATGTATTACTGTGCAAGAGGCGGGTCTACT | which is removed in the mature expressed |
| | ATGATTACGACGGGGCTGGGGTTTGCTTACTGGGGCCAAGGGACTCTGGTCACTGTCTCTGCACAAAACGAC | protein. |
| | ACCCCCATCTGTCTATCCACTGGCCCCTGGATCTGCTGCCCAAACTAACTCCATGGTGACCCTGGGATGCCT | |
| | GGTCAAGGGCTATTTCCCTGAGCCAGTGACAGTGACCTGGAACTCTGGATCCCTGTCCAGCGGTGTGCACAC | |
| | CTTCCCAGCTGTCCTGCAGTCTGACCTCTACACTCTGAGCAGCTCAGTGACTGTCCCCTCCAGCACCTGGCC | |
| | CAGCGAGACCGTCACCTGCAACGTTGCCCACCCGGCCAGCAGCACCAAGGTGGACAAGAAAATTGTGCCCAG | |
| | GGATTGTGGTTGTAAGCCTTGCATATGTACAGTCCCAGAAGTATCATCTGTCTTCATCTTCCCCCCAAAGCC | |
| | CAAGGATGTGCTCACCATTACTCTGACTCCTAAGGTCACGTGTGTTGTGGTAGACATCAGCAAGGATGATCC | |
| | CGAGGTCCAGTTCAGCTGGTTTGTAGATGATGTGGAGGTGCACACAGCTCAGACGCAACCCCGGGAGGAGCA | |
| | GTTCAACAGCACTTTCCGCTCAGTCAGTGAACTTCCCATCATGCACCAGGACTGGCTCAATGGCAAGGAGTT | |
| | CAAATGCAGGGTCAACAGTGCAGCTTTCCCTGCCCCCATCGAGAAAACCATCTCCAAAACCAAAGGCAGACC | |
| | GAAGGCTCCACAGGTGTACACCATTCCACCTCCCAAGGAGCAGATGGCCAAGGATAAAGTCAGTCTGACCTG | |
| | CATGATAACAGACTTCTTCCCTGAAGACATTACTGTGGAGTGGCAGTGGAATGGGCAGCCAGCGGAGAACTA | |
| | CAAGAACACTCAGCCCATCATGGACACAGATGGCTCTTACTTCATCTACAGCAAGCTCAATGTGCAGAAGAG | |
| | CAACTGGGAGGCAGGAAATACTTTCACCTGCTCTGTGTTACATGAGGGCCTGCACAACCACCATACTGAGAA | |
| | GAGCCTCTCCCACTCTCCTGGTAAATGA | |
| SEQ ID NO: 148 | ATGAAGTTGCCTGTTAGGCTGTTGGTGCTGATGGTCTGGATTCCTGCTTCCAGCAGTCAAATTGTTCTCACC | Nucleic acid sequence encoding the complete |
| | CAGTCTCCAGCAATCATGTCTGCATCTCCAGGGGAGAAGGTCACCATGACCTGCAGTGCCAGCTCAAGTGTA | sequence of the light chain of 1G9 as defined by |
| | AGTTACATGCACTGGTACCAGCAGAAGTCAGGCACCTCCCCCAAAAGATGGATTTATGACACATCCAAACTG | SEQ ID NO: 150. The nucleic sequence that is |
| | GCTTCTGGAGTCCCTGCTCGCTTCAGTGGCAGTGGGTCTGGGACCTCTTACTCTCTCACAATCAGCAGCATG | underlined encodes the leader sequence, which |
| | GAGGCTGAAGATGCTGCCACTTATTACTGCCAGCAGTGGAGTAGTAACCCACCCCTCACGTTCGGTGCTGGG | is removed in the mature expressed protein. |
| | ACCAAGCTGGAGCTGAAACGGGCTGATGCTGCACCAACTGTATCCATCTTCCCACCATCCAGTGAGCAGTTA | |
| | ACATCTGGAGGTGCCTCAGTCGTGTGCTTCTTGAACAACTTCTACCCCAAAGACATCAATGTCAAGTGGAAG | |
| | ATTGATGGCAGTGAACGACAAAATGGCGTCCTGAACAGTTGGACTGATCAGGACAGCAAAGACAGCACCTAC | |
| | AGCATGAGCAGCACCCTCACGTTGACCAAGGACGAGTATGAACGACATAACAGCTATACCTGTGAGGCCACT | |
| | CACAAGACATCAACTTCACCCATTGTCAAGAGCTTCAACAGGAATGAGTGTTAG | |
| SEQ ID NO: 149 | MKLWLNWIFLVTLLNGIQCEVKLVESGGGLVKPGGSLKLSCAASGFTFSSYAMSWVRQTPEKRLEWVASISS | The complete sequence of the heavy chain of |
| | GGSTYYPDSVKGRFTISRQNARNILYLQMSSLRSEDTAMYYCARGGSTMITTGLGFAYWGQGTLVTVSAAKT | 1G9. The leader sequence is underlined; it is |
| | TPPSVYPLAPGSAAQTNSMVTLGCLVKGYFPEPVTVTWNSGSLSSGVHTFPAVLQSDLYTLSSSVTVPSSTW | removed in the mature sequence. The mature V_{H} |
| | PSETVTCNVAHPASSTKVDKKIVPRDCGCKPCICTVPEVSSVFIFPPKPKDVLTITLTPKVTCVVVDISKDD | sequence is in bold. |
| | PEVQFSWFVDDVEVHTAQTQPREEQFNSTFRSVSELPIMHQDWLNGKEFKCRVNSAAFPAPIEKTISKTKGR | |
| | PKAPQVYTIPPPKEQMAKDKVSLTCMITDFFPEDITVEWQWNGQPAENYKNTQPIMDTDGSYFIYSKLNVQK | |
| | SNWEAGNTFTCSVLHEGLANHATEKSLSHSPGK | |
| SEQ ID NO: 150 | MKLPVRLLVLMVWIPASSSQIVLTQSPAIMSASPGEKVTMTCSASSSVSYMHWYQQKSGTSPKRWIYDTSKL | The complete sequence of the light chain of 1G9. |
| | ASGVPARFSGSGSGTSYSLTISSMEAEDAATYYCQQWSSNPPLTFGAGTKLELKRADAAPTVSIFPPSSEQL | The leader sequence is underlined; it is removed |
| | TSGGASVVCFLNNFYPKDINVKWKIDGSERQNGVLNSWTDQDSKDSTYSMSSTLTLTKDEYERHNSYTCEAT | in the mature sequence. The mature V_{L} sequence |
| | HKTSTSPIVKSFNRNEC | is in bold. |
| SEQ ID NO: 151 | ATGAAGTTGTGGCTGAACTGGATTTTCCTTGTAACACTTTTAAATGGTATCCAGTGTGAAGTGAAGCTGGTG | Nucleic acid sequence encoding the complete |
| | GAGTCTGGGGGAGACTTAGTGAAGCCTGGAGGGTCCCTGAAACTCTCCTGTGCAGCCTCTGGATTCACTTTC | sequence of the heavy chain of 1E8, as defined |
| | AGTAGCTATGCCATGTCTTGGGTTCGCCAGACTCCAGAGAAGAGGCTGGAGTGGGTCGCATCCATTAGTAGT | by SEQ ID NO: 153. The nucleic sequence that |
| | GGTGGTAGAACCTACTATCCAGACAGTGTGAAGGGCCGATTCACCATCTCCAGAGATAATGCCAGGAACATC | is underlined encodes the leader sequence, |
| | CTGTACCTGCAAATGAGCAGTCTGAGGTCTGAAGACACGGCCATCTATTACTGTGCACGAGGCGGGACTCGT | which is removed in the mature expressed |
| | CATTCCTACGGCAACGGGTTTGACTACTGGGGCCAAGGCACCACTCTCACAGTCTCCTCACAAAACGACACC | protein. |
| | CCCATCTGTCTATCCACTGGCCCCTGGATCTGCTGCCCAAACTAACTCCATGGTGACCCTGGGATGCCTGGT | |
| | CAAGGGCTATTTCCCTGAGCCAGTGACAGTGACCTGGAACTCTGGATCCCTGTCCAGCGGTGTGCACACCTT | |
| | CCCAGCTGTCCTGCAGTCTGACCTCTACACTCTGAGCAGCTCAGTGACTGTCCCCTCCAGCACCTGGCCCAG | |
| | CGAGACCGTCACCTGCAACGTTGCCCACCCGGCCAGCAGCACCAAGGTGGACAAGAAAATTGTGCCCAGGGA | |
| | TTGTGGTTGTAAGCCTTGCATATGTACAGTCCCAGAAGTATCATCTGTCTTCATCTTCCCCCCAAAGCCCAA | |
| | GGATGTGCTCACCATTACTCTGACTCCTAAGGTCACGTGTGTTGTGGTAGACATCAGCAAGGATGATCCCGA | |
| | GGTCCAGTTCAGCTGGTTTGTAGATGATGTGGAGGTGCACACAGCTCAGACGCAACCCCGGGAGGAGCAGTT | |
| | CAACAGCACTTTCCGCTCAGTCAGTGAACTTCCCATCATGCACCAGGACTGGCTCAATGGCAAGGAGTTCAA | |
| | ATGCAGGGTCAACAGTGCAGCTTTCCCTGCCCCCATCGAGAAAACCATCTCCAAAACCAAAGGCAGACCGAA | |
| | GGCTCCACAGGTGTACACCATTCCACCTCCCAAGGAGCAGATGGCCAAGGATAAAGTCAGTCTGACCTGCAT | |
| | GATAACAGACTTCTTCCCTGAAGACATTACTGTGGAGTGGCAGTGGAATGGGCAGCCAGCGGAGAACTACAA | |
| | GAACACTCAGCCCATCATGGACACAGATGGCTCTTACTTCATCTACAGCAAGCTCAATGTGCAGAAGAGCAA | |
| | CTGGGAGGCAGGAAATACTTTCACCTGCTCTGTGTTACATGAGGGCCTGCACAACCACCATACTGAGAAGAG | |
| | CCTCTCCCACTCTCCTGGTAAATGA | |
| SEQ ID NO: 152 | ATGAAGTTGCCTGTTAGGCTGTTGGTGCTGATGGTCTGGATTCCTGCTTCCAGCAGTCAAATTGTTCTCTCC | Nucleic acid sequence encoding the complete |
| | CAGTCTCCAACAATCATGTCTGCATCTCCAGGGGAGAAGGTCACCATGACCTGCAGTGCCAGCTCAAGTGTA | sequence of the light chain of 1E8 as defined by |
| | AGTTACATGCACTGGTACCAGCAGAAGTCAGGCACCTCCCCCAAAAGATGGATTTATGACTCATCCAAACTG | SEQ ID NO: 154. The nucleic sequence that is |
| | GCTTCTGGAGTCCCTGCTCGCTTCAGTGGCAGTGGGTCTGGGACCTCTTACTCTCTCACAATCAGCAGCATG | underlined encodes the leader sequence, which |
| | GAGGCTGAAGATGCTGCCACTTATTACTGCCAGCAGTGGACTAGTAACCCACCCATCACGTTCGGTGCTGGG | is removed in the mature expressed protein. |
| | ACCAAGCTGGAGCTGAAACGGGCTGATGCTGCACCAACTGTATCCATCTTCCCACCATCCAGTGAGCAGTTA | |
| | ACATCTGGAGGTGCCTCAGTCGTGTGCTTCTTGAACAACTTCTACCCCAAAGACATCAATGTCAAGTGGAAG | |
| | ATTGATGGCAGTGAACGACAAAATGGCGTCCTGAACAGTTGGACTGATCAGGACAGCAAAGACAGCACCTAC | |
| | AGCATGAGCAGCACCCTCACGTTGACCAAGGACGAGTATGAACGACATAACAGCTATACCTGTGAGGCCACT | |
| | CACAAGACATCAACTTCACCCATTGTCAAGAGCTTCAACAGGAATGAGTGTTAG | |
| SEQ ID NO: 153 | MKLWLNWIFLVTLLNGIQCEVKLVESGGDLVKPGGSLKLSCAASGFTFSSYAMSWVRQTPEKRLEWVASISS | The complete sequence of the heavy chain of |
| | GGRTYYPDSVKGRFTISRDNARNILYLQMSSLRSEDTAIYYCARGGTRHSYGNGFDYWGQGTTLTVSSAKTT | 1E8. The leader sequence is underlined; it is |
| | PPSVYPLAPGSAAQTNSMVTLGCLVKGYFPEPVTVTWNSGSLSSGVHTFPAVLQSDLYTLSSSVTVPSSTWP | removed in the mature sequence. The mature V_{H} |
| | SETVTCNVAHPASSTKVDKKIVPRDCGCKPCICTVPEVSSVFIFPPKPKDVLTITLTPKVTCVVVDISKDDP | sequence is in bold. |
| | EVQFSWFVDDVEVHTAQTQPREEQFNSTFRSVSELPIMHQDWLNGKEFKCRVNSAAFPAPIEKTISKTKGRP | |
| | KAPQVYTIPPPKEQMAKDKVSLTCMITDFFPEDITVEWQWNGQPAENYKNTQPIMDIDGSYFIYSKLNVQKS | |
| | NWEAGNTFTCSVLHEGLHNHHTEKSLSHSPGK | |
| SEQ ID NO: 154 | MKLPVRLLVLMVWIPASSSQIVLSQSPTIMSASPGEKVTMTCSASSSVSYMHWYQQKSGTSPKRWIYDSSKL | The complete sequence of the light chain of 1E8. |
| | ASGVPARFSGSGSGTSYSLTISSMEAEDAATYYCQQWTSNPPITFGAGTKLELKRADAAPTVSIFPPSSEQL | The leader sequence is underlined; it is removed |
| | TSGGASVVCFLNNFYPKDINVKWKIDGSERQNGVLNSWTDQDSKDSTYSMSSTLTLTKDEYERHNSYTCEAT | in the mature sequence. The mature Vi sequence |
| | HKTSTSPIVKSFNRNEC | is in bold. |
| SEQ ID NO: 155 | ATGAAGTTGTGGCTGAACTGGATTTTCCTTGTAACACTTTTAAATGGTATCCAGTGTGAAGTGAAACTGGTT | Nucleic acid sequence encoding the complete |
| | GAGTCTGGGGGAGTCTTAGTGAAGCCTGGAGGGTCCCTGAAATTTTCCTGTGCAGCCTCTGGATTCACTTTC | sequence of the heavy chain of 1C10, as defined |
| | AGTAGCCATGCCTTGTCTTGGGTTCGCCAGACTCCAGAGAAGAGACTGGAGTGGGTCGCATCCATTAGTAGT | by SEQ ID NO: 157. The nucleic sequence that |
| | CGTGGTCGAACCTACTATCCAGACAGTGTAAAGGGCCGATTCACCGTCTCCAGAGATAATGCCAGGAACATC | is underlined encodes the leader sequence, |
| | CTGTATCTGCAAGTGAGCAGTCTGAGGTCTGAGGACACGGCCATGTATTATTGTACAAGAGGCGGGACTCAT | which is removed in the mature expressed |
| | TATTCCTACGGCAACGGTTTTGACTTCTGGGGCCAAGGCACCACTCTCACAGTCTCCTCACAAAACGACACC | protein. |
| | CCCATCTGTCTATCCACTGGCCCCTGGATCTGCTGCCCAAACTAACTCCATGGTGACCCTGGGATGCCTGGT | |
| | CAAGGGCTATTTCCCTGAGCCAGTGACAGTGACCTGGAACTCTGGATCCCTGTCCAGCGGTGTGCACACCTT | |
| | CCCAGCTGTCCTGCAGTCTGACCTCTACACTCTGAGCAGCTCAGTGACTGTCCCCTCCAGCACCTGGCCCAG | |
| | CGAGACCGTCACCTGCAACGTTGCCCACCCGGCCAGCAGCACCAAGGTGGACAAGAAAATTGTGCCCAGGGA | |
| | TTGTGGTTGTAAGCCTTGCATATGTACAGTCCCAGAAGTATCATCTGTCTTCATCTTCCCCCCAAAGCCCAA | |
| | GGATGTGCTCACCATTACTCTGACTCCTAAGGTCACGTGTGTTGTGGTAGACATCAGCAAGGATGATCCCGA | |
| | GGTCCAGTTCAGCTGGTTTGTAGATGATGTGGAGGTGCACACAGCTCAGACGCAACCCCGGGAGGAGCAGTT | |
| | CAACAGCACTTTCCGCTCAGTCAGTGAACTTCCCATCATGCACCAGGACTGGCTCAATGGCAAGGAGTTCAA | |
| | ATGCAGGGTCAACAGTGCAGCTTTCCCTGCCCCCATCGAGAAAACCATCTCCAAAACCAAAGGCAGACCGAA | |
| | GGCTCCACAGGTGTACACCATTCCACCTCCCAAGGAGCAGATGGCCAAGGATAAAGTCAGTCTGACCTGCAT | |
| | GATAACAGACTTCTTCCCTGAAGACATTACTGTGGAGTGGCAGTGGAATGGGCAGCCAGCGGAGAACTACAA | |
| | GAACACTCAGCCCATCATGGACACAGATGGCTCTTACTTCATCTACAGCAAGCTCAATGTGCAGAAGAGCAA | |
| | CTGGGAGGCAGGAAATACTTTCACCTGCTCTGTGTTACATGAGGGCCTGCACAACCACCATACTGAGAAGAG | |
| | CCTCTCCCACTCTCCTGGTAAATGA | |
| SEQ ID NO: 156 | ATGAAGTTGCCTGTTAGGCTGTTGGTGCTGATGGTCTGGATTCCTGCTTCCAGCAGTCAAATTGTTCTCACC | Nucleic acid sequence encoding the complete |
| | CAGTCTCCAGCAATCATGTCTGCATCTCCAGGGGAGAAGGTCACCATGACCTGCAGTGTCAGCTCAAGTGTT | sequence of the light chain of 1C10 as defined by |
| | AGTTACATGCACTGGTACCAGCAGAAGTCAGGCACCTCCCCCAAAAGATGGATTTATGACACATCCAAACTG | SEQ ID NO: 158. The nucleic sequence that is |
| | GCTTCTGGAGTCCCTGCTCGCTTCAGTGGCAGTGGGTCTGGGACCTCTTACTCTCTCACAATCAGCACCATG | underlined encodes the leader sequence, which |
| | GAGGCTGAAGATGCTGCCACTTATTACTGCCAGCAGTGGAGTAATAACCCACCCATCACGTTCGGTGCTGGG | is removed in the mature expressed protein. |
| | ACCAAGCTGGAACTGAAACGGGCTGATGCTGCACCAACTGTATCCATCTTCCCACCATCCAGTGAGCAGTTA | |
| | ACATCTGGAGGTGCCTCAGTCGTGTGCTTCTTGAACAACTTCTACCCCAAAGACATCAATGTCAAGTGGAAG | |
| | ATTGATGGCAGTGAACGACAAAATGGCGTCCTGAACAGTTGGACTGATCAGGACAGCAAAGACAGCACCTAC | |
| | AGCATGAGCAGCACCCTCACGTTGACCAAGGACGAGTATGAACGACATAACAGCTATACCTGTGAGGCCACT | |
| | CACAAGACATCAACTTCACCCATTGTCAAGAGCTTCAACAGGAATGAGTGTTAG | |
| SEQ ID NO: 157 | MKLWLNWIFLVTLLNGIQCEVKLVESGGVLVKPGGSLKFSCAASGFTFSSHALSWVRQTPEKRLEWVASISS | The complete sequence of the heavy chain of |
| | RGRTYYPDSVKGRFTVSRDNARNILYLQVSSLRSEDTAMYYCTRGGTHYSYGNGFDFWGQGTTLTVSSAKTT | 1C10. The leader sequence is underlined; it is |
| | PPSVYPLAPGSAAQTNSMVTLGCLVKGYFPEPVTVTWNSGSLSSGVHTFPAVLQSDLYTLSSSVTVPSSTWP | removed in the mature sequence. The mature V_{H} |
| | SETVTCNVAHPASSTKVDKKIVPRDCGCKPCICTVPEVSSVFIFPPKPKDVLTITLTPKVTCVVVDISKDDP | sequence is in bold. |
| | EVQESWEVDDVEVHTAQTQPREEQFNSTFRSVSELPIMHQDWLNGKEFKCRVNSAAFPAPIEKTISKTKGRP | |
| | KAPQVYTIPPPKEQMAKDKVSLTCMITDFFPEDITVEWQWNGQPAENYKNTQPIMDTDGSYFIYSKLNVQKS | |
| | NWEAGNTFTCSVLHEGLHNHHTEKSLSHSPGK | |
| SEQ ID NO: 158 | MKLPVRLLVLMVWIPASSSQIVLTQSPAIMSASPGEKVTMTCSVSSSVSYMHWYQQKSGTSPKRWIYDTSKL | The complete sequence of the light chain of |
| | ASGVPARFSGSGSGTSYSLTISTMEAEDAATYYCQQWSNNPPITFGAGTKLELKRADAAPTVSIFPPSSEQL | 1C10. The leader sequence is underlined; it is |
| | TSGGASVVCFLNNFYPKDINVKWKIDGSERQNGVLNSWTDQDSKDSTYSMSSTLTLTKDEYERHNSYTCEAT | removed in the mature sequence. The mature V_{L} |
| | HKTSTSPIVKSFNRNEC | sequence is in bold. |
| SEQ ID NO: 159 | ATGAAGTTGTGGCTGAACTGGATTTTCCTTGTAACACTTTTAAATGGTATCCAGTGTGAAGTGAAACTGGTT | Nucleic acid sequence encoding the complete |
| | GAGTCTGGGGGAGTCTTAGTGAAGCCTGGAGGGTCCCTGAAATTTTCCTGTGCAGCCTCTGGATTCACTCTC | sequence of the heavy chain of 1A10, as defined |
| | AGTAGCCATGCCTTGTCTTGGGTTCGCCAGACTCCAGAGAAGAGACTGGAGTGGGTCGCATCCATTAGTAGT | by SEQ ID NO: 161. The nucleic sequence that |
| | CGTGGTCGAACCTACTATCCAGACAGTGTAAAGGGCCGATTCACCGTCTCCAGAGATAATGCCAGGAACATC | is underlined encodes the leader sequence, |
| | CTGTATCTGCAAGTGAGCAGTCTGAGGTCTGAGGACACGGCCATGTATTATTGTACAAGAGGCGGGACTCAT | which is removed in the mature expressed |
| | TATTCCTACGGCAACGGTTTTGACTTCTGGGGCCAAGGCACCACTCTCACAGTCTCCTCACAAAACGACACC | protein. |
| | CCCATCTGTCTATCCACTGGCCCCTGGATCTGCTGCCCAAACTAACTCCATGGTGACCCTGGGATGCCTGGT | |
| | CAAGGGCTATTTCCCTGAGCCAGTGACAGTGACCTGGAACTCTGGATCCCTGTCCAGCGGTGTGCACACCTT | |
| | CCCAGCTGTCCTGCAGTCTGACCTCTACACTCTGAGCAGCTCAGTGACTGTCCCCTCCAGCACCTGGCCCAG | |
| | CGAGACCGTCACCTGCAACGTTGCCCACCCGGCCAGCAGCACCAAGGTGGACAAGAAAATTGTGCCCAGGGA | |
| | TTGTGGTTGTAAGCCTTGCATATGTACAGTCCCAGAAGTATCATCTGTCTTCATCTTCCCCCCAAAGCCCAA | |
| | GGATGTGCTCACCATTACTCTGACTCCTAAGGTCACGTGTGTTGTGGTAGACATCAGCAAGGATGATCCCGA | |
| | GGTCCAGTTCAGCTGGTTTGTAGATGATGTGGAGGTGCACACAGCTCAGACGCAACCCCGGGAGGAGCAGTT | |
| | CAACAGCACTTTCCGCTCAGTCAGTGAACTTCCCATCATGCACCAGGACTGGCTCAATGGCAAGGAGTTCAA | |
| | ATGCAGGGTCAACAGTGCAGCTTTCCCTGCCCCCATCGAGAAAACCATCTCCAAAACCAAAGGCAGACCGAA | |
| | GGCTCCACAGGTGTACACCATTCCACCTCCCAAGGAGCAGATGGCCAAGGATAAAGTCAGTCTGACCTGCAT | |
| | GATAACAGACTTCTTCCCTGAAGACATTACTGTGGAGTGGCAGTGGAATGGGCAGCCAGCGGAGAACTACAA | |
| | GAACACTCAGCCCATCATGGACACAGATGGCTCTTACTTCATCTACAGCAAGCTCAATGTGCAGAAGAGCAA | |
| | CTGGGAGGCAGGAAATACTTTCACCTGCTCTGTGTTACATGAGGGCCTGCACAACCACCATACTGAGAAGAG | |
| | CCTCTCCCACTCTCCTGGTAAATGA | |
| SEQ ID NO: 160 | ATGAAGTTGCCTGTTAGGCTGTTGGTGCTGATGGTCTGGATTCCTGCTTCCAGCAGTCAAATTGTTCTCACC | Nucleic acid sequence encoding the complete |
| | CAGTCTCCAGCAATCATGTCTGCATCTCCAGGGGAGAAGGTCACCATGACCTGCAGTGTCAGCTCAAGTGTT | sequence of the light chain of 1A10 as defined by |
| | AGTTACATGCACTGGTACCAGCAGAAGTCAGGCACCTCCCCCAAAAGATGGATTTATGACACATCCAAACTG | SEQ ID NO: 162. The nucleic sequence that is |
| | GCTTCTGGAGTCCCTGCTCGCTTCAGTGGCAGTGGGTCTGGGACCTCTTACTCTCTCACAATCAGCACCATG | underlined encodes the leader sequence, which |
| | GAGGCTGAAGATGCTGCCACTTATTACTGCCAGCAGTGGAGTAATAACCCACCCATCACGTTCGGTGCTGGG | is removed in the mature expressed protein. |
| | ACCAAGCTGGAACTGAAACGGGCTGATGCTGCACCAACTGTATCCATCTTCCCACCATCCAGTGAGCAGTTA | |
| | ACATCTGGAGGTGCCTCAGTCGTGTGCTTCTTGAACAACTTCTACCCCAAAGACATCAATGTCAAGTGGAAG | |
| | ATTGATGGCAGTGAACGACAAAATGGCGTCCTGAACAGTTGGACTGATCAGGACAGCAAAGACAGCACCTAC | |
| | AGCATGAGCAGCACCCTCACGTTGACCAAGGACGAGTATGAACGACATAACAGCTATACCTGTGAGGCCACT | |
| | CACAAGACATCAACTTCACCCATTGTCAAGAGCTTCAACAGGAATGAGTGTTAG | |
| SEQ ID NO: 161 | MKLWLNWIFLVTLLNGIQCEVKLVESGGVLVKPGGSLKFSCAASGFTLSSHALSWVRQTPEKRLEWVASISS | The complete sequence of the heavy chain of |
| | RGRTYYPDSVKGRFTVSRDNARNILYLQVSSLRSEDTAMYYCTRGGTHYSYGNGFDFWGQGTTLTVSSAKTT | 1A10. The leader sequence is underlined; it is |
| | PPSVYPLAPGSAAQTNSMVTLGCLVKGYFPEPVTVTWNSGSLSSGVHTFPAVLQSDLYTLSSSVTVPSSTWP | removed in the mature sequence. The mature V_{H} |
| | SETVTCNVAHPASSTKVDKKIVPRDCGCKPCICTVPEVSSVFIFPPKPKDVLTITLTPKVTCVVVDISKDDP | sequence is in bold. |
| | EVQESWFVDDVEVHTAQTQPREEQFNSTFRSVSELPIMHQDWLNGKEFKQRVNSAAFPAPIEKTISKTKGRP | |
| | KAPQVYTIPPPKEQMAKDKVSLTCMITDFFPEDITVEWQWNGQPAENYKNTQPIMDTDGSYFIYSKLNVQKS | |
| | NWEAGNTFTCSVLHEGLHNHHTEKSLSHSPGK | |
| SEQ ID NO: 162 | MKLPVRLLVLMVWIPASSSQIVLTQSPAIMSASPGEKVTMTCSVSSSVSYMHWYQQKSGTSPKRWIYDTSKL | The complete sequence of the light chain of |
| | ASGVPARFSGSGSGTSYSLTISTMEAEDAATYYCQQWSNNPPITFGAGTKLELKRADAAPTVSIFPPSSEQL | 1A10. The leader sequence is underlined; it is |
| | TSGGASVVCFLNNFYPKDINVKWKIDGSERQNGVLNSWTDQDSKDSTYSMSSTLTLTKDEYERINSYTCEAT | removed in the mature sequence. The mature V_{L} |
| | HKTSTSPIVKSFNRNEC | sequence is in bold. |
| SEQ ID NO: 167 | S(Y/H)A(M/L)S | ECD3 V_{H}-CDR1 consensus sequence |
| SEQ ID NO: 168 | SISS(G/R)G(S/R)TYYPDSVKG | ECD3 V_{H}-CDR2 consensus sequence, variant 1 |
| SEQ ID NO: 169 | GG(S/T)(T/R/H)(M/H/Y)(I/S)(T/Y)(T/G)(G/N)(L/*)GF(A/D)(Y/F) | ECD3 V_{H}-CDR3 consensus sequence, variant 1 |
| SEQ ID NO: 170 | S(A/V)SSSVSYMH | ECD3 V_{L}-CDR1 consensus sequence |
| SEQ ID NO: 171 | D(T/S)SKLAS | ECD3 V_{L}-CDR2 consensus sequence |
| SEQ ID NO: 172 | QQW(S/T/*)SN(*/N)PP(I/L)T | ECD3 V_{L}-CDR3 consensus sequence, variant 1 |
| SEQ ID NO: 173 | MTQTTT TELTTEFDYDLGAALCTLTDVLNQSKPITLFLYGVVFLFGSIGNFLVIFTITWRRRIQCSGDVYF | Mutated strain of US28 (artificially created). Note |
| | INLAAADLLFVCTLPLWMQYLLDANSLASVPCTLLTACFYVAMFASLCFITEIALDRYYAIVYMRYRPVKQA | that the space indicates a deletion made for the |
| | CLFSIFWWIFAVIIAIPHFMVVTKKDNQCMTDYDYLEVSYPIILNVELMLGAFVIPLSVISYCYYRISRIVA | mutated sequence (which is not present in the |
| | VSQSRHKGRIVRVLIAVVLVFIIFWLPYHLTLFLDTLKLLKWISSSCESEKSLKRALILTESLAFCHCCLNP | version of this sequence in the sequence listing). |
| | LLYVFVGTKFRQELHCLLAEFRQRLFSRDVSWYHSMSFSRRSSPSRRETSSDTLSDEVCRVSQIIP | The underlined sections, in order, correspond to |
| | | the N-terminus (i.e. ECD1), ECD2, ECD3 and |
| | | ECD4, respectively. Amino acids with bold |
| | | underlining and emboldened text indicate |
| | | positions that have been mutated |
| SEQ ID NO: 174 | SISS(G/R)GRTYYPDSVKG | ECD3 V_{H}-CDR2 consensus sequence, variant 2 |
| SEQ ID NO: 175 | GG(S/T)(T/R/H)(M/H/Y)(I/S)(T/Y) (T/G)(G/N)GE(A/D)(Y/F) | ECD3 V_{H}-CDR3 consensus sequence, variant 2 |
| SEQ ID NO: 176 | QQW(T/*)SN(*/N)PPIT | V_{L}-CDR3 consensus sequence, variant 2 |
| SEQ ID NO: 177 | TDVLNQSKPVTL | Conserved epitope of ECD1 of the US28 protein |
| SEQ ID NO: 178 | GCGGCCGCAAACTACAAGACAGACTTGCAAAAGAAGGCATGCACAGCTCAGCACTGCTCTGTTGCCTGGTCC | Nucleic acid sequence encoding complete |
| | TCCTGACTGGGGTGAGGGCCCAGGTGCAGCTGCAGCAGTCTGGAGCTGAACTGGCTAAACCCGGCGCTTCTG | sequence of the heavy chain of 4H3C3 |
| | TGAAAATGTCTTGTAAAGCCTCAGGCTATACCTTCACATCCTACTGGATGCACTGGGTGAAACAGAGACCCG | |
| | GCCAGGGACTGGAATGGATTGGATACATCAACCCCTCAACCGGCTACGCCGAGTATAACCAGAAATTCAAGG | |
| | ACAAGGCTACCQTGACCGCCGACAAAAGCAGCTCAACCGCTTACATGCAGCTGAGCAGCCTGACCAGCGAGG | |
| | ATTCTGCTGTGTACTACTGCGCCAGACTGAGATTCGGCAGCAGCGGATTCGCCTACTGGGGACAGGGAACAC | |
| | TGGTGACAGTGTCTTCCGCTAAAACGACACCCCCATCTGTCTATCCACTGGCCCCTGGATCTGCTGCCCAAA | |
| | CTAACTCCATGGTGACCCTGGGATGCCTGGTCAAGGGCTATTTCCCTGAGCCAGTGACAGTGACCTGGAACT | |
| | CTGGATCCCTGTCCAGCGGTGTGCACACCTTCCCAGCTGTCCTGCAGTCTGACCTCTACACTCTGAGCAGCT | |
| | CAGTGACTGTCCCCTCCAGCACCTGGCCCAGCGAGACCGTCACCTGCAACGTTGCCCACCCGGCCAGCAGCA | |
| | CCAAGGTGGACAAGAAAATTGTGCCCAGGGATTGTGGTTGTAAGCCTTGCATATGTACAGTCCCAGAAGTAT | |
| | CATCTGTCTTCATCTTCCCCCCAAAGCCCAAGGATGTGCTCACCATTACTCTGACTCCTAAGGTCACGTGTG | |
| | TTGTGGTAGACATCAGCAAGGATGATCCCGAGGTCCAGTTCAGCTGGTTTGTAGATGATGTGGAGGTGCACA | |
| | CAGCTCAGACGCAACCCCGGGAGGAGCAGTTCAACAGCACTTTCCGCTCAGTCAGTGAACTTCCCATCATGC | |
| | ACCAGGACTGGQTCAATGGCAAGGAGTTCAAATGCAGGGTCAACAGTGCAGCTTTCCCTGCCCCCATCGAGA | |
| | AAACCATCTCCAAAACCAAAGGCAGACCGAAGGCTCCGCAGGTGTACACCATTCCACCTQCCAAGGAGCAGA | |
| | TGGCCAAGGATAAAGTCAGTCTGACCTGCATGATAACAGACTTCTTCCCTGAAGACATTACTGTGGAGTGGC | |
| | AGTGGAATGGGCAGCCAGCGGAGAACTACAAGAACACTCAGCCCATCATGGACACAGATGGCTCTTACTTCA | |
| | TCTACAGCAAGCTCAATGTGCAGAAGAGCAACTGGGAGGCAGGAAATACTTTCACCTGCTCTGTGTTACATG | |
| | AGGGCCTGCACAACCACCATACTGAGAAGAGCCTCTCCCACTCTCCTGGTAAATGATTCTAGA | |
| SEQ ID NO: 179 | GCGGCCGCAAACTACAAGACAGACTTGCAAAAGAAGGCATGCACAGCTCAGCACTGCTCTGTTGCCTGGTCC | Nucleic acid sequence encoding complete |
| | TCCTGACTGGGGTGAGGGCCCAGATCGTGCTGACCCAGTCACCAAGCAGCCTGGCTGTGTCTGTGGGAGAAA | sequence of the light chain of 4H3C3 |
| | AGGTGACCATGAGCTGCAAAAGCTCACAGAGCCTGCTGTACAGQTCCAACCAGAAGAACTACCTGGCCTGGT | |
| | ATCAGCAGAAACCCGGCCAGTCACCCAAACTGCTGATCTATTGGGCCAGCACCTGGGAGAGCGGAGTGCCTG | |
| | ATAGATTCACCGGGTCCGGAAGCGGAACAGACTTCACACTGACCATTAGCAGCGTGAAAGCTGAGGACCTGG | |
| | CCGTGTACTACTGCCAGCAGTACTACAGCTTTCCCCTGACCTTTGGAGCCGGCACAAAGCTGGAGATCAAGA | |
| | GAGCAGATGCTGCACCAACTGTATCCATCTTCCCACCATCCAGTGAGCAGTTAACATCTGGAGGTGCCTCAG | |
| | TCGTGTGCTTCTTGAACAACTTCTACCCCAAAGACATCAATGTCAAGTGGAAGATTGATGGCAGTGAACGAC | |
| | AAAATGGCGTCCTGAACAGTTGGACTGATCAGGACAGCAAAGACAGCACCTACAGCATGAGCAGCACCCTCA | |
| | CGTTGACCAAGGACGAGTATGAACGACATAACAGCTATACCTGTGAGGCCACTCACAAGACATCAACTTCAC | |
| | CCATTGTCAAGAGCTTCAACAGGAATGAGTGTTGATTCTAGA | |
| SEQ ID NO: 180 | GRKLQDRLAKEGMHSSALLCCLVLLTGVRAQVQLQQSGAELAKPGASVKMSCKASGYTFTSYWMHWVKQRPG | Complete amino acid sequence of the heavy |
| | QGLEWIGYINPSTGYAEYNQKFKDKATLTADKSSSTAYMQLSSLTSEDSAVYYCARLRFGSSGFAYWGQGTL | chain of 4H3C3 |
| | VTVSSAKTTPPSVYPLAPGSAAQTNSMVTLGCLVKGYFPEPVTVTWNSGSLSSGVHTFPAVLQSDLYTLSSS | |
| | VTVPSSTWPSETVTCNVAHPASSTKVDKKIVPRDCGCKPCICTVPEVSSVFIFPPKPKDVLTITLTPKVTCV | |
| | VVDISKDDPEVQFSWFVDDVEVHTAQTQPREEQFNSTFRSVSELPIMHQDWINGKEFKCRVNSAAFPAPIEK | |
| | TISKTKGRPKAPQVYTIPPPKEQMAKDKVSLTCMITDFFPEDITVEWQWNGQPAENYKNTQPIMDTDGSYFI | |
| | YSKINVQKSNWEAGNTFTCSVLHEGLHNHHTEKSLSHSPGK | |
| SEQ ID NO: 181 | GRKLQDRLAKEGMASSALLCCLVLLTGVRAQIVLTQSPSSLAVSVGEKVTMSCKSSQSLLYSSNQKNYLAWY | Complete amino acid sequence of the light chain |
| | QQKPGQSPKLLIYWASTWESGVPDRFTGSGSGTDFTLTISSVKAEDLAVYYCQQYYSFPLTFGAGTKLEIKR | of 4H3C3 |
| | ADAAPTVSIFPPSSEQLTSGGASVVCFLNNFYPKDINVKWKIDGSERQNGVLNSWTDQDSKDSTYSMSSTLT | |
| | LTKDEYERHNSYTCEATHKTSTSPIVKSFNRNEC | |
| SEQ ID NO: 182 | GCGGCCGCAAACTACAAGACAGACTTGCAAAAGAAGGCATGCACAGCTCAGCACTGCTCTGTTGCCTGGTCC | Nucleic acid sequence encoding immature form |
| | TCCTGACTGGGGTGAGGGCCCAGGTGCAGCTGCAGCAGTCTGGAGCTGAACTGGCTAAACCCGGCGQTTCTG | of the recombinantly expressed VH sequence of |
| | TGAAAATGTCTTGTAAAGCCTCAGGCTATACCTTCACATCCTACTGGATGCACTGGGTGAAACAGAGACCCG | 4H3C3 |
| | GCCAGGGACTGGAATGGATTGGATACATCAACCCCTCAACCGGCTACGCCGAGTATAACCAGAAATTCAAGG | |
| | ACAAGGCTACCCTGACCGCCGACAAAAGCAGCTCAACCGCTTACATGCAGCTGAGCAGCCTGACCAGCGAGG | |
| | ATTCTGCTGTGTACTACTGCGCCAGACTGAGATTCGGCAGCAGCGGATTCGCCTACTGGGGACAGGGAACAC | |
| | TGGTGACAGTGTCTTCC | |
| SEQ ID NO: 183 | CAGGTGCAGCTGCAGCAGTCTGGAGCTGAACTGGCTAAACCCGGCGCTTCTGTGAAAATGTCTTGTAAAGCC | Nucleic acid sequence encoding mature form of |
| | TCAGGCTATACCTTCACACCTACTGGATGCACTGGGTGAAACAGAGACCCGGCCAGGGACTGGAATGGATT | VH sequence of 4H3C3 |
| | GGATACATCAACCCCTCAACCGGCTACGCCGAGTATAACCAGAAATTCAAGGACAAGGCTACCCTGACCGCC | |
| | GACAAAAGCAGCTCAACCGCTTACATGCAGCTGAGCAGCCTGACCAGCGAGGATTCTGCTGTGTACTACTGC | |
| | GCCAGACTGAGATTCGGCAGCAGCGGATTCGCCTACTGGGGACAGGGAACACTGGTGACAGTGTCTTCC | |
| SEQ ID NO: 184 | GCGGCCGCAAACTACAAGACAGACTTGCAAAAGAAGGCATGCACAGCTCAGCACTGCTCTGTTGCCTGGTCC | Nucleic acid sequence encoding immature form |
| | TCCTGACTGGGGTGAGGGCCCAGATCGTGCTGACCCAGTCACCAAGCAGCCTGGCTGTGTCTGTGGGAGAAA | of the recombinantly expressed VL sequence of |
| | AGGTGACCATGAGCTGCAAAAGCTCACAGAGCCTGCTGTACAGCTCCAACCAGAAGAACTACCTGGCCTGGT | 4H3C3 |
| | ATCAGCAGAAACCCGGCCAGTCACCCAAACTGCTGATCTATTGGGCCAGCACCTGGGAGAGCGGAGTGCCTG | |
| | ATAGATTCACCGGGTCCGGAAGCGGAACAGACTTCACACTGACCATTAGCAGCGTGAAAGCTGAGGACCTGG | |
| | CCGTGTACTACTGCCAGCAGTACTACAGCTTTCCCCTGACCTTTGGAGCCGGCACAAAGCTGGAGATCAAG | |
| SEQ ID NO: 185 | CAGATCGTGCTGACCCAGTCACCAAGCAGCCTGGCTGTGTCTGTGGGAGAAAAGGTGACCATGAGCTGCAAA | Nucleic acid sequence encoding mature form of |
| | AGCTCACAGAGCCTGCTGTACAGCTCCAACCAGAAGAACTACCTGGCCTGGTATCAGCAGAAACCCGGCCAG | VL sequence of 4H3C3 |
| | TCACCCAAACTGCTGATCTATTGGGCCAGCACCTGGGAGAGCGGAGTGCCTGATAGATTCACCGGGTCCGGA | |
| | AGCGGAACAGACTTCACACTGACCATTAGCAGCGTGAAAGCTGAGGACCTGGCCGTGTACTACTGCCAGCAG | |
| | TACTACAGCTTTCCCCTGACCTTTGGAGCCGGCACAAAGCTGGAGATCAAG | |
| SEQ ID NO: 186 | GRKLQDRLAKEGMHSSALLCCLVLLTGVRAQVQLQQSGAELAKPGASVKMSCKASGYTFTSYWMHWVKQRPG | Immature form of the recombinantly expressed |
| | QGLEWIGYINPSTGYAEYNQKFKDKATLTADKSSSTAYMQLSSLTSEDSAVYYCARLRFGSSGFAYWGQGTL | VH sequence of 4H3C3, wherein positions 1-19 |
| | VTVSS | correspond to a leader sequence |
| SEQ ID NO: 187 | QVQLQQSGAELAKPGASVKMSCKASGYIFTSYWMHWVKQRPGQGLEWIGYINPSTGYAEYNQKKDKATLTA | Mature form of VH sequence of 4H3C3, without |
| | DKSSSTAYMQLSSITSEDSAVYYCARLRFGSSGFAYWGQGTLVTVSS | the leader sequence |
| SEQ ID NO: 188 | GRKLQDRLAKEGMHSSALLCCLVLLTGVRAQIVLTQSPSSLAVSVGEKVTMSCKSSQSLLYSSNQKNYLAWY | Immature form of the recombinantly expressed |
| | QQKPGQSPKLLIYWASTWESGVPDRFTGSGSGTDFTLTISSVKAEDLAVYYCQQYYSFPLTFGAGTKLEIK | VL sequence of 4H3C3, wherein positions 1-19 |
| | | correspond to a leader sequence |
| SEQ ID NO: 189 | QIVLTQSPSSLAVSVGEKVTMSCKSSQSLLYSSNQKNYLAWYQQKPGQSPKLLIYWASTWESGVPDRFTGSG | Mature form of VL sequence of 4H3C3, without |
| | SGTDFTLTISSVKAEDLAVYYCQQYYSFPLTFGAGTKLEIK | the leader sequence |
| SEQ ID NO: 190 | TCCTACTGGATGCAC | Nucleic acid sequence encoding VH-CDR1 |
| | | sequence of 4H3C3 |
| SEQ ID NO: 191 | TACATCAACCCCTCAACCGGCTACGCCGAGTATAACCAGAAATTCAAGGAC | Nucleic acid sequence encoding VH-CDR2 |
| | | sequence of 4H3C3 |
| SEQ ID NO: 192 | CTGAGATTCGGCAGCAGCGGATTCGCCTAC | Nucleic acid sequence encoding VH-CDR3 |
| | | sequence of 4H3C3 |
| SEQ ID NO: 193 | AAAAGCTCACAGAGCCTGCTGTACAGCTCCAACCAGAAGAACTACCTGGCC | Nucleic acid sequence encoding VL-CDR1 |
| | | sequence of 4H3C3 |
| SEQ ID NO: 194 | TGGGCCAGCACCTGGGAGAGC | Nucleic acid sequence encoding VL-CDR2 |
| | | sequence of 4H3C3 |
| SEQ ID NO: 195 | CAGCAGTACTACAGCTTTCCCCTGACC | Nucleic acid sequence encoding VL-CDR3 |
| | | sequence of 4H3C3 |
| SEQ ID NO: 196 | SYWMH | VH-CDR1 amino acid sequence of 4H3C3 |
| SEQ ID NO: 197 | YINPSTGYAEYNQKFKD | VH-CDR2 amino acid sequence of 4H3C3 |
| SEQ ID NO: 198 | LRFGSSGFAY | VH-CDR3 amino acid sequence of 4H3C3 |
| SEQ ID NO: 199 | KSSQSLLYSSNQKNYLA | VL-CDR1 amino acid sequence of 4H3C3, 781F3 |
| | | and 2F5B11 |
| SEQ ID NO: 200 | WASTWES | VL-CDR2 amino acid sequence of 4H3C3 |
| SEQ ID NO: 201 | QQYYSFPLT | VL-CDR3 amino acid sequence of 4H3C3 |
| SEQ ID NO: 202 | GCGGCCGCAAACTACAAGACAGACTTGCAAAAGAAGGCATGCACAGCTCAGCACTGCTCTGTTGCCTGGTCC | Nucleic acid sequence encoding complete |
| | TCCTGACTGGGGTGAGGGCCGAAGTGCAGCTGCAGCAGAGTGGAGCTGAGCTGGCTAAGCCTGGAGCTAGTG | sequence of the heavy chain of 7B1F3 |
| | TGAAGATGAGCTGCAAGGCCAGCGGATACACATTTACCAACTACTGGATGCACTGGGTGAAGCAGAGACCCG | |
| | GACAGGGACTGGAATGGATTGGATACGTGAACCCCAGAACAGGATACACCGAGTACAACCAGAAGTTCAAAG | |
| | ACAAAGCCACCCTGACCGCCGACAAAAGCAGCAGAACCGCTTACATGCAGCTGTCCAGCCTGACCAGCGAGG | |
| | ATTCTGCTGTGTACAGTTGCGCCAGAATCCTGAACGGCACCGGATTTGCCTACTGGGGACAGGGAACCCTGG | |
| | TGACAGTGTCTGCTGCTAAAACGACACCCCCATCTGTCTATCCACTGGCCCCTGGATCTGCTGCCCAAACTA | |
| | ACTCCATGGTGACCCTGGGATGCCTGGTCAAGGGCTATTTCCCTGAGCCAGTGACAGTGACCTGGAACTCTG | |
| | GATCCCTGTCCAGCGGTGTGCACACCTTCCCAGCTGTCCTGCAGTCTGACCTCTACACTCTGAGCAGCTCAG | |
| | TGACTGTCCCCTCCAGCACCTGGCCCAGCGAGACCGTCACCTGCAACGTTGCCCACCCGGCCAGCAGCACCA | |
| | AGGTGGACAAGAAAATTGTGCCCAGGGATTGTGGTTGTAAGCCTTGCATATGTACAGTCCCAGAAGTATCAT | |
| | CTGTCTTCATCTTCCCCCCAAAGCCCAAGGATGTGCTCACCATTACTCTGACTCCTAAGGTCACGTGTGTTG | |
| | TGGTAGACATCAGCAAGGATGATCCCGAGGTCCAGTTCAGCTGGTTTGTAGATGATGTGGAGGTGCACACAG | |
| | CTCAGACGCAACCCCGGGAGGAGCAGTTCAACAGCACTTTCCGCTCAGTCAGTGAACTTCCCATCATGCACC | |
| | AGGACTGGCTCAATGGCAAGGAGTTCAAATGCAGGGTCAACAGTGCAGCTTTCCCTGCCCCCATCGAGAAAA | |
| | CCATCTCCAAAACCAAAGGCAGACCGAAGGCTCCGCAGGTGTACACCATTCCACCTCCCAAGGAGCAGATGG | |
| | CCAAGGATAAAGTCAGTCTGACCTGCATGATAACAGACTTCTTCCCTGAAGACATTACTGTGGAGTGGCAGT | |
| | GGAATGGGCAGCCAGCGGAGAACTACAAGAACACTCAGCCCATCATGGACACAGATGGCTCTTACTTCATCT | |
| | ACAGCAAGCTCAATGTGCAGAAGAGCAACTGGGAGGCAGGAAATACTTTCACCTGCTCTGTGTTACATGAGG | |
| | GCCTGCACAACCACCATACTGAGAAGAGCCTCTCCCACTCTCCTGGTAAATGATTCTAGA | |
| SEQ ID NO: 203 | GCGGCCGCAAACTACAAGACAGACTTGCAAAAGAAGGCATGCACAGCTCAGCACTGCTCTGTTGCCTGGTCC | Nucleic acid sequence encoding complete |
| | TCCTGACTGGGGTGAGGGCCGATATTGTGCTGACCCAGAGCCCCAGCAGCCTGACAGTGTCTGTGGGAGAAA | sequence of the light chain of 7B1F3 |
| | AGTTTACCATGAGCTGTAAGAGCAGCCAGAGCCTGCTGTACAGCAGCAACCAGAAGAACTACCTGGCCTGGT | |
| | ATCAGCAGAAGCCCGGACAGTCACCCAAGCTGCTGATTTACTGGGCCAGCACCCGGGAAAGCGGAGTGCCTG | |
| | ATAGATTCACCGGCAGCGGAAGCGGAACCGATTTCACACTGGCTATCAGCAGCGTGAAGCCCGAGGATCTGG | |
| | CTATCTACTACTGCCAGCAGTACTACAGCTTCCCACTGACCTTCGGCGCCGGAACAAAGCTGGAAATCAAGA | |
| | GAGCAGATGCTGCACCAACTGTATCCATCTTCCCACCATCCAGTGAGCAGTTAACATCTGGAGGTGCCTCAG | |
| | TCGTGTGCTTCTTGAACAACTTCTACCCCAAAGACATCAATGTCAAGTGGAAGATTGATGGCAGTGAACGAC | |
| | AAAATGGCGTCCTGAACAGTTGGACTGATCAGGACAGCAAAGACAGCACCTACAGCATGAGCAGCACCCTCA | |
| | CGTTGACCAAGGACGAGTATGAACGACATAACAGCTATACCTGTGAGGCCACTCACAAGACATCAACTTCAC | |
| | CCATTGTCAAGAGCTTCAACAGGAATGAGTGTTGATTCTAGA | |
| SEQ ID NO: 204 | GRKLQDRLAKEGMESSALLCCLVLLTGVRAEVQLQQSGAELAKPGASVKMSCKASGYTFTNYWMHWVKQRPG | Complete amino acid sequence of the heavy |
| | QGLEWIGYVNPRTGYTEYNQKFKDKATLTADKSSRTAYMQLSSLTSEDSAVYSCARILNGTGFAYWGQGTLV | chain of 781F3 |
| | TVSAAKTTPPSVYPLAPGSAAQTNSMVTLGCLVKGYFPEPVTVTWNSGSLSSGVHTFPAVLQSDLYTLSSSV | |
| | TVPSSTWPSETVTCNVAHPASSTKVDKKIVPRDCGCKPCICTVPEVSSVFIFPPKPKDVLTITLTPKVTCVV | |
| | VDISKDDPEVQFSWFVDDVEVHTAQTQPREEQFNSTFRSVSELPIMHQDWLNGKEFKCRVNSAAFPAPIEKT | |
| | ISKTKGRPKAPQVYTIPPPKEQMAKDKVSLTCMITDFFPEDITVEWQWNGQPAENYKNTQPIMDTDGSYFIY | |
| | SKLNVQKSNWEAGNTFTCSVLHEGLHNHHTEKSLSHSPGK | |
| SEQ ID NO: 205 | GRKLQDRLAKEGMHSSALLCCLVLLTGVRADIVLTQSPSSLTVSVGEKFTMSCKSSQSLLYSSNQKNYLAWY | Complete amino acid sequence of the light chain |
| | QQKPGQSPKLLIYWASTRESGVPDRFTGSGSGTDFTLAISSVKPEDLAIYYCQQYYSFPLTFGAGTKLEIKR | of 7B1F3 |
| | ADAAPTVSIFPPSSEQLTSGGASVVCFLNNFYPKDINVKWKIDGSERQNGVLNSWTDQDSKDSTYSMSSTLT | |
| | LTKDEYERHNSYTCEATHKTSTSPIVKSFNRNEC | |
| SEQ ID NO: 206 | GCGGCCGCAAACTACAAGACAGACTTGCAAAAGAAGGCATGCACAGCTCAGCACTGCTCTGTTGCCTGGTCC | Nucleic acid sequence encoding immature form |
| | TCCTGACTGGGGTGAGGGCCGAAGTGCAGCTGCAGCAGAGTGGAGCTGAGCTGGCTAAGCCTGGAGCTAGTG | of the recombinantly expressed VH sequence of |
| | TGAAGATGAGCTGCAAGGCCAGCGGATACACATTTACCAACTACTGGATGCACTGGGTGAAGCAGAGACCCG | 781F3 |
| | GACAGGGACTGGAATGGATTGGATACGTGAACCCCAGAACAGGATACACCGAGTACAACCAGAAGTTCAAAG | |
| | ACAAAGCCACCCTGACCGCCGACAAAAGCAGCAGAACCGQTTACATGCAGCTGTCCAGCCTGACCAGCGAGG | |
| | ATTCTGCTGTGTACAGTTGCGCCAGAATCCTGAACGGCACCGGATTTGCCTACTGGGGACAGGGAACCCTGG | |
| | TGACAGTGTCTGCT | |
| SEQ ID NO: 207 | GAAGTGCAGCTGCAGCAGAGTGGAGCTGAGCTGGCTAAGCCTGGAGCTAGTGTGAAGATGAGCTGCAAGGCC | Nucleic acid sequence encoding mature form of |
| | AGCGGATACACATTTACCAACTACTGGATGCACTGGGTGAAGCAGAGACCCGGACAGGGACTGGAATGGATT | VH sequence of 7B1F3 |
| | GGATACGTGAACCCCAGAACAGGATACACCGAGTACAACCAGAAGTTCAAAGACAAAGCCACCCTGACCGCC | |
| | GACAAAAGCAGCAGAACCGCTTACATGCAGCTGTCCAGCCTGACCAGCGAGGATTCTGCTGTGTACAGTTGC | |
| | GCCAGAATCCTGAACGGCACCGGATTTGCCTACTGGGGACAGGGAACCCTGGTGACAGTTGTCTGCT | |
| SEQ ID NO: 208 | GCGGCCGCAAACTACAAGACAGACTTGCAAAAGAAGGCATGCACAGCTCAGCACTGCTCTGTTGCCTGGTCC | Nucleic acid sequence encoding immature form |
| | TCCTGACTGGGGTGAGGGCCGATATTGTGCTGACCCAGAGCCCCAGCAGCCTGACAGTGTCTGTGGGAGAAA | of the recombinantly expressed VL sequence of |
| | AGTTTACCATGAGCTGTAAGAGCAGCCAGAGCCTGCTGTACAGCAGCAACCAGAAGAACTACCTGGCCTGGT | 7B1F3 |
| | ATCAGCAGAAGCCCGGACAGTCACCCAAGCTGCTGATTTACTGGGCCAGCACCCGGGAAAGCGGAGTGCCTG | |
| | ATAGATTCACCGGCAGCGGAAGCGGAACCGATTTCACACTGGCTATCAGCAGCGTGAAGCCCGAGGATCTGG | |
| | CTATCTACTACTGCCAGCAGTACTACAGCTTCCCACTGACCTTCGGCGCCGGAACAAAGCTGGAAATCAAG | |
| SEQ ID NO: 209 | GATATTGTGCTGACCCAGAGCCCCAGCAGCCTGACAGTGTCTGTGGGAGAAAAGTTTACCATGAGCTGTAAG | Nucleic acid sequence encoding mature form of |
| | AGCAGCCAGAGCCTGCTGTACAGCAGCAACCAGAAGAACTACCTGGCCTGGTATCAGCAGAAGCCCGGACAG | VL sequence of 7B1F3 |
| | TCACCCAAGCTGCTGATTTACTGGGCCAGCACCCGGGAAAGCGGAGTGCCTGATAGATTCACCGGCAGCGGA | |
| | AGCGGAACCGATTTCACACTGGCTATCAGCAGCGTGAAGCCCGAGGATCTGGCTATCTACTACTGCCAGCAG | |
| | TACTACAGCTTCCCACTGACCTTCGGCGCCGGAACAAAGCTGGAAATCAAG | |
| SEQ ID NO: 210 | GRKLQDRLAKEGMESSALLCCLVLLTGVRAEVQLQQSGAELAKPGASVKMSCKASGYTFTNYWMHWVKQRPG | Immature form of the recombinantly expressed |
| | QGLEWIGYVNPRTGYTEYNQKEKDKATLIADKSSRTAYMQLSSLTSEDSAVYSCARILNGTGFAYWGQGTLV | VH sequence of 781F3, wherein positions 1-19 |
| | TVSA | correspond to a leader sequence |
| SEQ ID NO: 211 | EVQLQQSGAELAKPGASVKMSCKASGYTFTNYWMHWVKQRPGQGLEWIGYVNPRTGYTEYNQKEKDKATLTA | Mature form of VH sequence of 7B1F3, without |
| | DKSSRTAYMQLSSITSEDSAVYSCARILNGTGFAYWGQGTLVTVSA | the leader sequence |
| SEQ ID NO: 212 | GRKLQDRLAKEGMHSSALLCCLVLLTGVRADIVLTQSPSSLTVSVGEKFTMSCKSSQSLLYSSNQKNYLAWY | Immature form of the recombinantly expressed |
| | QQKPGQSPKLLIYWASTRESGVPDRFTGSGSGTDFTLAISSVKPEDLAIYYCQQYYSFPLTFGAGTKLEIK | VL sequence of 7B1F3, wherein positions 1-19 |
| | | correspond to a leader sequence |
| SEQ ID NO: 213 | DIVLTQSPSSLIVSVGEKFTMSCKSSQSLLYSSNQKNYLAWYQQKPGQSPKLLIYWASTRESGVPDRFTGSG | Mature form of VL sequence of 7B1F3, without |
| | SGTDFTLAISSVKPEDLAIYYCQQYYSFPLTFGAGTKLEIK | the leader sequence |
| SEQ ID NO: 214 | AACTACTGGATGCAC | Nucleic acid sequence encoding VH-CDR1 |
| | | sequence of 7B1F3 |
| SEQ ID NO: 215 | TACGTGAACCCCAGAACAGGATACACCGAGTACAACCAGAAGTTCAAAGAC | Nucleic acid sequence encoding VH-CDR2 |
| | | sequence of 7B1F3 |
| SEQ ID NO: 216 | ATCCTGAACGGCACCGGATTTGCCTAC | Nucleic acid sequence encoding VH-CDR3 |
| | | sequence of 7B1F3 |
| SEQ ID NO: 217 | TGGGCCAGCACCCGGGAAAGC | Nucleic acid sequence encoding VL-CDR2 |
| | | sequence of 7B1F3 |
| SEQ ID NO: 218 | CAGCAGTACTACAGCTTCCCACTGACC | Nucleic acid sequence encoding VL-CDR3 |
| | | sequence of 7B1F3 |
| SEQ ID NO: 219 | NYWMH | VH-CDR1 amino acid sequence of 7B1F3 |
| SEQ ID NO: 220 | YVNPRTGYTEYNQKFKD | VH-CDR2 amino acid sequence of 7B1F3 |
| SEQ ID NO: 221 | ILNGTGFAY | VH-CDR3 amino acid sequence of 7B1F3 |
| SEQ ID NO: 222 | WASTRES | VL-CDR2 amino acid sequence of 7B1F3 |
| SEQ ID NO: 223 | QQYYSFPLT | VL-CDR3 amino acid sequence of 7B1F3 |
| SEQ ID NO: 224 | GCGGCCGCAAACTACAAGACAGACTTGCAAAAGAAGGCATGCACAGCTCAGCACTGCTCTGTTGCCTGGTCC | Nucleic acid sequence encoding VL complete |
| | TCCTGACTGGGGTGAGGGCCGAAGTGCAGCTGCAGGAAAGCGGAGQTGAGCTGGCTAAGCCTGGAGCTTCTG | sequence of the heavy chain of 2F5B11 |
| | TGAAGATGAGCTGTAAGGCCAGCGGATACACATTTAGTAGCTATTGGATTCATTGGATCAAGCAGAGACCTG | |
| | GCCAGGGACTGGAATGGATTGGATACATCAACCCCAACACCGCCTATACCGAGTTCAACCAGAAATTCATGG | |
| | ACAAGGCCACCCTGACCGCCGATAAAAGCAGCTCAACCGCCTACATGCAGCTGACCAGCCTGACAAGCGAAG | |
| | ACAGCGCTGTGTACTATTGCGCCAGGCTGAGAAGCGACAGAACCGGATTCGCCTACTGGGGACAGGGAACAC | |
| | TGGTGACAGTGAGTGCCGCTAAAACGACACCCCCATCTGTCTATCCACTGGCCCCTGGATCTGCTGCCCAAA | |
| | CTAACTCCATGGTGACCCTGGGATGCCTGGTCAAGGGCTATTTCCCTGAGCCAGTGACAGTGACCTGGAACT | |
| | CTGGATCCCTGTCCAGCGGTGTGCACACCTTCCCAGCTGTCCTGCAGTCTGACCTCTACACTCTGAGCAGCT | |
| | CAGTGACTGTCCCCTCCAGCACCTGGCCCAGCGAGACCGTCACCTGCAACGTTGCCCACCCGGCCAGCAGCA | |
| | CCAAGGTGGACAAGAAAATTGTGCCCAGGGATTGTGGTTGTAAGCCTTGCATATGTACAGTCCCAGAAGTAT | |
| | CATCTGTCTTCATCTTCCCCCCAAAGCCCAAGGATGTGCTCACCATTACTCTGACTCCTAAGGTCACGTGTG | |
| | TTGTGGTAGACATCAGCAAGGATGATCCCGAGGTCCAGTTCAGCTGGTTTGTAGATGATGTGGAGGTGCACA | |
| | CAGCTCAGACGCAACCCCGGGAGGAGCAGTTCAACAGCACTTTCCGCTCAGTCAGTGAACTTCCCATCATGC | |
| | ACCAGGACTGGCTCAATGGCAAGGAGTTCAAATGCAGGGTCAACAGTGCAGCTTTCCCTGCCCCCATCGAGA | |
| | AAACCATCTCCAAAACCAAAGGCAGACCGAAGGCTCCGCAGGTGTACACCATTCCACCTCCCAAGGAGCAGA | |
| | TGGCCAAGGATAAAGTCAGTCTGACCTGCATGATAACAGACTTCTTCCCTGAAGACATTACTGTGGAGTGGC | |
| | AGTGGAATGGGCAGCCAGCGGAGAACTACAAGAACACTCAGCCCATCATGGACACAGATGGCTCTTACTTCA | |
| | TCTACAGCAAGCTCAATGTGCAGAAGAGCAACTGGGAGGCAGGAAATACTTTCACCTGCTCTGTGTTACATG | |
| | AGGGCCTGCACAACCACCATACTGAGAAGAGCCTCTCCCACTCTCCTGGTAAATGATTCTAGA | |
| SEQ ID NO: 225 | GCGGCCGCAAACTACAAGACAGACTTGCAAAAGAAGGCATGCACAGCTCAGCACTGCTCTGTTGCCTGGTCC | Nucleic acid sequence encoding VL complete |
| | TCCTGACTGGGGTGAGGGCCCAGATCGTGCTGACCCAGTCACCAAGCAGCCTGGCTGTGTCTGTGGGAGAAA | sequence of the light chain of 2F5B11 |
| | AGGTGACCATGAGCTGCAAAAGCTCACAGAGCCTGCTGTACAGCTCCAACCAGAAGAACTACCTGGCCTGGT | |
| | ATCAGCAGAAACCCGGCCAGTCACCCAAAGTGCTGATCTACTGGGCCAGCACCAGAGAATACGGCGTGCCTG | |
| | ATAGATTCACCGGCAGCGGAAGCGGAACCGATTTCACACTGACTATTAGCAGCGTGAAGGCTGAAGACCTGG | |
| | CCGTGTACTACTGCCAGCAGTACTACAGCTTCCCCCTGACCTTTGGAGCCGGAACAAAGCTGGAAATCAAGA | |
| | GAGCAGATGCTGCACCAACTGTATCCATQTTCCCACCATCCAGTGAGCAGTTAACATCTGGAGGTGCCTCAG | |
| | TCGTGTGCTTCTTGAACAACTTCTACCCCAAAGACATCAATGTCAAGTGGAAGATTGATGGCAGTGAACGAC | |
| | AAAATGGCGTCCTGAACAGTTGGACTGATCAGGACAGCAAAGACAGCACCTACAGCATGAGCAGCACCCTCA | |
| | CGTTGACCAAGGACGAGTATGAACGACATAACAGCTATACCTGTGAGGCCACTCACAAGACATCAACTTCAC | |
| | CCATTGTCAAGAGCTTCAACAGGAATGAGTGTTGATTCTAGA | |
| SEQ ID NO: 226 | GRKLQDRLAKEGMHSSALLCCLVLLTGVRAEVQLQESGAELAKPGASVKMSCKASGYTFSSYWIHWIKQRPG | Complete sequence of the heavy chain of |
| | QGLEWIGYINPNTAYTEFNQKFMDKATLIADKSSSTAYMQLTSLTSEDSAVYYCARLRSDRTGFAYWGQGTL | 2F5B11 |
| | VTVSAAKTIPPSVYPLAPGSAAQTNSMVILGCLVKGYFPEPVTVTWNSGSLSSGVHTEPAVLQSDLYTLSSS | |
| | VTVPSSTWPSETVTCNVAHPASSTKVDKKIVPRDCGCKPCICTVPEVSSVFIFPPKPKDVLTITLTPKVTCV | |
| | VVDISKDDPEVQFSWFVDDVEVHTAQTQPREEQFNSTFRSVSELPIMHQDWLNGKEFKCRVNSAAFPAPIEK | |
| | TISKTKGRPKAPQVYTIPPPKEQMAKDKVSLTCMITDFFPEDITVEWQWNGQPAENYKNTQPIMDIDGSYFI | |
| | YSKINVQKSNWEAGNTFTCSVLHEGLANHHTEKSLSHSPGK | |
| SEQ ID NO: 227 | GRKLQDRLAKEGMHSSALLCCLVLLTGVRAQIVLTQSPSSLAVSVGEKVTMSCKSSQSLLYSSNQKNYLAWY | Complete sequence of the light chain of 2F5B11 |
| | QQKPGQSPKVLIYWASTREYGVPDRFTGSGSGTDFTLTISSVKAEDLAVYYCQQYYSFPLTFGAGTKLEIKR | |
| | ADAAPTVSIFPPSSEQLTSGGASVVCFLNNFYPKDINVKWKIDGSERQNGVLNSWTDQDSKDSTYSMSSTLT | |
| | LTKDEYERHNSYTCEATHKTSTSPIVKSFNRNEC | |
| SEQ ID NO: 228 | GCGGCCGCAAACTACAAGACAGACTTGCAAAAGAAGGCATGCACAGCTCAGCACTGCTCTGTTGCCTGGTCC | Nucleic acid sequence encoding VL immature |
| | TCCTGACTGGGGTGAGGGCCGAAGTGCAGCTGCAGGAAAGCGGAGCTGAGCTGGCTAAGCCTGGAGCTTCTG | form of the recombinantly expressed VH |
| | TGAAGATGAGCTGTAAGGCCAGCGGATACACATTTAGTAGCTATTGGATTCATTGGATCAAGCAGAGACCTG | sequence of 2F5B11 |
| | GCCAGGGACTGGAATGGATTGGATACATCAACCCCAACACCGCCTATACCGAGTTCAACCAGAAATTCATGG | |
| | ACAAGGCCACCCTGACCGCCGATAAAAGCAGCTCAACCGCCTACATGCAGCTGACCAGCCTGACAAGCGAAG | |
| | ACAGCGCTGTGTACTATTGCGCCAGGCTGAGAAGCGACAGAACCGGATTCGCCTACTGGGGACAGGGAACAC | |
| | TGGTGACAGTGAGTGCC | |
| SEQ ID NO: 229 | GAAGTGCAGCTGCAGGAAAGCGGAGCTGAGCTGGCTAAGCCTGGAGCTTCTGTGAAGATGAGCTGTAAGGCC | Nucleic acid sequence encoding VL mature form |
| | AGCGGATACACATTTAGTAGCTATTGGATTCATTGGATCAAGCAGAGACCTGGCCAGGGACTGGAATGGATT | of VH sequence of 2F5B11 |
| | GGATACATCAACCCCAACACCGCCTATACCGAGTTCAACCAGAAATTCATGGACAAGGCCACCCTGACCGCC | |
| | GATAAAAGCAGCTCAACCGCCTACATGCAGCTGACCAGCCTGACAAGCGAAGACAGCGCTGTGTACTATTGC | |
| | GCCAGGCTGAGAAGCGACAGAACCGGATTCGCCTACTGGGGACAGGGAACACTGGTGACAGTGAGTGCC | |
| SEQ ID NO: 230 | GCGGCCGCAAACTACAAGACAGACTTGCAAAAGAAGGCATGCACAGCTCAGCACTGCTCTGTTGCCTGGTCC | Nucleic acid sequence encoding VL immature |
| | TCCTGACTGGGGTGAGGGCCCAGATCGTGCTGACCCAGTCACCAAGCAGCCTGGCTGTGTCTGTGGGAGAAA | form of the recombinantly expressed VL |
| | AGGTGACCATGAGCTGCAAAAGCTCACAGAGCCTGCTGTACAGCTCCAACCAGAAGAACTACCTGGCCTGGT | sequence of 2F5B11 |
| | ATCAGCAGAAACCCGGCCAGTCACCCAAAGTGCTGATCTACTGGGCCAGCACCAGAGAATACGGCGTGCCTG | |
| | ATAGATTCACCGGCAGCGGAAGCGGAACCGATTTCACACTGACTATTAGCAGCGTGAAGGCTGAAGACCTGG | |
| | CCGTGTACTACTGCCAGCAGTACTACAGCTTCCCCCTGACCTTTGGAGCCGGAACAAAGCTGGAAATCAAG | |
| SEQ ID NO: 231 | CAGATCGTGCTGACCCAGTCACCAAGCAGCCTGGCTGTGTCTGTGGGAGAAAAGGTGACCATGAGCTGCAAA | Nucleic acid sequence encoding VL mature form |
| | AGCTCACAGAGCCTGCTGTACAGCTCCAACCAGAAGAACTACCTGGCCTGGTATCAGCAGAAACCCGGCCAG | of VL sequence of 2F5B11 |
| | TCACCCAAAGTGCTGATCTACTGGGCCAGCACCAGAGAATACGGCGTGCCTGATAGATTCACCGGCAGCGGA | |
| | AGCGGAACCGATTTCACACTGACTATTAGCAGCGTGAAGGCTGAAGACCTGGCCGTGTACTACTGCCAGCAG | |
| | TACTACAGCTTCCCCCTGACCTTTGGAGCCGGAACAAAGCTGGAAATCAAG | |
| SEQ ID NO: 232 | GRKLQDRLAKEGMHSSALLCCLVLLTGVRAEVQLQESGAELAKPGASVKMSCKASGYTFSSYWIHWIKQRPG | Immature form of the recombinantly expressed |
| | QGLEWIGYINPNTAYTEFNQKEMDKATLTADKSSSTAYMQLTSLISEDSAVYYCARLRSDRTGFAYWGQGTL | VH sequence of 2F5B11, wherein positions 1-19 |
| | VTVSA | correspond to a leader sequence |
| SEQ ID NO: 233 | EVQLQESGAELAKPGASVKMSCKASGYTFSSYWIHWIKQRPGQGLEWIGYINPNTAYTEFNQKEMDKATLTA | Mature form of VH sequence of 2F5B11, without |
| | DKSSSTAYMQLTSLISEDSAVYYCARLRSDRTGFAYWGQGTLVTVSA | the leader sequence |
| SEQ ID NO: 234 | GRKLQDRLAKEGMHSSALLCCLVLLTGVRAQIVLTQSPSSLAVSVGEKVTMSCKSSQSLLYSSNQKNYLAWY | Immature form of the recombinantly expressed |
| | QQKPGQSPKVLIYWASTREYGVPDRFTGSGSGTDFTLTISSVKAEDLAVYYCQQYYSFPLTFGAGTKLEIK | VL sequence of 2F5B11, wherein positions 1-19 |
| | | correspond to a leader sequence |
| SEQ ID NO: 235 | QIVLTQSPSSLAVSVGEKVTMSCKSSQSLLYSSNQKNYLAWYQQKPGQSPKVLIYWASTREYGVPDRFTGSG | Mature form of VL sequence of 2F5B11, without |
| | SGTDETLTISSVKAEDLAVYYCQQYYSFPLTFGAGTKLEIK | the leader sequence |
| SEQ ID NO: 236 | AGCTATTGGATTCAT | Nucleic acid sequence encoding VH-CDR1 |
| | | sequence of 2F5B11 |
| SEQ ID NO: 237 | TACATCAACCCCAACACCGCCTATACCGAGTTCAACCAGAAATTCATGGAC | Nucleic acid sequence encoding VH-CDR2 |
| | | sequence of 2F5B11 |
| SEQ ID NO: 238 | CTGAGAAGCGACAGAACCGGATTCGCCTAC | Nucleic acid sequence encoding VH-CDR3 |
| | | sequence of 2F5B11 |
| SEQ ID NO: 239 | AAAAGCTCACAGAGCCTGCTGTACAGCTCCAACCAGAAGAACTACCTGGCC | Nucleic acid sequence encoding VL-CDR1 |
| | | sequence of 2F5B11 |
| SEQ ID NO: 240 | TGGGCCAGCACCAGAGAATAC | Nucleic acid sequence encoding VL-CDR2 |
| | | sequence of 2F5B11 |
| SEQ ID NO: 241 | CAGCAGTACTACAGCTTCCCCCTGACC | Nucleic acid sequence encoding VL-CDR3 |
| | | sequence of 2F5B11 |
| SEQ ID NO: 242 | SYWIH | VH-CDR1 amino acid sequence of 2F5B11 |
| SEQ ID NO: 243 | YINPNTAYTEFNQKFMD | VH-CDR2 amino acid sequence of 2F5B11 |
| SEQ ID NO: 244 | LRSDRTGFAY | VH-CDR3 amino acid sequence of 2F5B11 |
| SEQ ID NO: 245 | WASTREY | VL-CDR2 amino acid sequence of 2F5B11 |
| SEQ ID NO: 246 | QQYYSFPLT | VL-CDR3 amino acid sequence of 2F5811 |
| SEQ ID NO: 247 | (S/N)YW(M/I)H | ECD1 V_{H}-CDR1 consensus sequence |
| SEQ ID NO: 248 | Y(I/V)NP(S/R/N)T(G/A)Y(A/T)E(Y/F)NQKT(K/M)D | ECD1 V_{H}-CDR2 consensus sequence |
| SEQ ID NO: 249 | (L/I)(R/L)(F/N/S)(G/D)(S/*/R)(S/T)GFAY | ECD1 V_{H}-CDR3 consensus sequence |
| SEQ ID NO: 250 | WAST(W/R)E(S/Y) | ECD1 V_{L}-CDR2 consensus sequence |
| SEQ ID NO: 251 | AAGAGCAGCCAGAGCCTGCTGTACAGCAGCAACCAGAAGAACTACCTGGCC | Nucleic acid sequence encoding VL-CDR1 |
| | | sequence of 7B1F3 |
| SEQ ID NO: 252 | GCGGCCGCAAACTACAAGACAGACTTGCAAAAGAAGGCATGCACAGCTCAGCACTGCTCTGTTGCCTGGTCC | Nucleic acid sequence encoding the leader |
| | TCCTGACTGGGGTGAGGGCC | sequence of recombinantly expressed VH |
| | | sequence of ECD1-binding molecules |
| SEQ ID NO: 253 | GRKLQDRLAKEGMHSSALLCCLVLLTGVRA | The leader sequence (also referred to as the |
| | | signal peptide) of recombinantly expressed VH |
| | | and Vi sequences of ECD1-binding molecules |
| SEQ ID NO: 254 | MTPTTTTTELTTEFEYDLGATPCTFTDVLNQSKPVTLFLYGVVFLFGSVGNFLVIFTITWRRRIQCSGDVYF | US28 fusion protein, comprising US28 sequence |
| | INLAAADLLFVCTLPLWMQYLLDANSLASVPCTLLTACFYVAMFASLCFITEIALDRYYAIVYMRYRPVKQA | from HCMV TB40/Estrain |
| | CLFSIFWWIFAVIIAIPHFMVVTKKDNQCMTDYDYLEVSYPIILNVELMLGAFVIPLSVISYCYYRISRIVA | |
| | VSQSRHKGRIVRVLIAVVLVFIIFWLPYHLTLFVDTLKLLKWISSSCEFERSLKRALILTESLAFCHCCLNP | |
| | LLYVFVGTKFRQELHCLLAEFRQRLFSRDVSWYHSMSFSRRSSPSRRETSSDTLSDEVCRVSQIIPAAALEG | |
| | SHHHHHH | |
| SEQ ID NO: 255 | MTQTTITELTTEFDYDLGAALCILTDVLNQSKPITLFLYGVVFLFGSIGNFLVIFTITWRRRIQCSGDVYFI | US28 fusion protein, comprising US28 sequence |
| | NLAAADLLFVCTLPLWMQYLLDANSLASVPCTLLTACFYVAMFASLCFITEIALDRYYAIVYMRYRPVKQAC | from HCMV mutated strain |
| | LFSIFWWIFAVIIAIPHFMVVTKKDNQCMTDYDYLEVSYPIILNVELMIGAFVIPLSVISYCYYRISRIVAV | |
| | SQSRHKGRIVRVLIAVVLVFIIFWLPYHLTLFLDTLKLLKWISSSCESEKSLKRALILTESLAFCHCCLNPL | |
| | LYVEVGTKFRQELHCLLAEFRQRLFSRDVSWYHSMSFSRRSSPSRRETSSDTLSDEVCRVSQIIPAAALEGS | |
| | HHHHHH | |
| SEQ ID NO: 256 | QVQLVQSGAEVKKPGASVKVSCKASGYTFTSYWMHWVRQAPGQGLEWIGYINPSTGYAEYNQKFQGRATLTA | 4H3C3 VH1 |
| | DKSSSTAYMELSSLRSEDTAVYYCARLRFGSSGFAYWGQGTLVTVSS | |
| SEQ ID NO: 257 | QVQLVQSGAEVKKPGASVKVSCKASGYTFTSYWMHWVRQAPGQGLEWMGYINPSTGYAEYNQKFQGRVTMTA | 4H3C3 VH2 |
| | DKSTSTAYMELSSLRSEDTAVYYCARLRFGSSGFAYWGQGTLVTVSS | |
| SEQ ID NO: 258 | QVQLVQSGAEVKKPGASVKVSCKASGYTFTSYWMHWVRQAPGQGLEWIGYINPSTGYAEYNQKLQGRATLTA | 4H3C3 VH3. |
| | DKSSSTAYMELRSLRSDDTAVYYCARLRFGSSGFAYWGQGTLVTVSS | |
| SEQ ID NO: 259 | QVQLVQSGAEVKKPGASVKVSCKASGYTFTSYWMHWVRQAPGQGLEWMGYINPSTGYAEYNQKLQGRVTMTA | 4H3C3 VH4. |
| | DKSTSTAYMELRSLRSDDTAVYYCARLRFGSSGFAYWGQGTLVTVSS | |
| SEQ ID NO: 260 | QIVLTQSPDSLAVSLGERATINCKSSQSLLYSSNQKNYLAWYQQKPGQPPKLLIYWASTWESGVPDRFSGSG | 4H3C3 VL1 |
| | SGTDFTLTISSLQAEDVAVYYCQQYYSFPLTFGGGTKLEIK | |
| SEQ ID NO: 261 | DIVLTQSPDSLAVSLGERATINCKSSQSLLYSSNQKNYLAWYQQKPGQPPKLLIYWASTRESGVPDRFSGSG | 4H3C3 VL2 and 7B1F3 VL2 |
| | SGIDFILTISSLQAEDVAVYYCQQYYSFPLTEGGGTKLEIK | |
| SEQ ID NO: 262 | QIVLTQSPLSLPVTPGEPASISCRSSQSLLYSSNQKNYLAWYLQKPGQSPQLLIYWASTWESGVPDRFSGSG | 4H3C3 VL3 |
| | SGTDFTLKISRVEAEDVGVYYCQQYYSEPLTFGGGTKLEIK | |
| SEQ ID NO: 263 | DIVLTQSPLSLPVTPGEPASISCRSSQSLLYSSNQKNYLAWYLQKPGQSPQLLIYWASTRESGVPDRFSGSG | 4H3C3 VL4 and 7B1F3 VL4 |
| | SGTDFTLKISRVEAEDVGVYYCQQYYSFPLTFGGGTKLEIK | |
| SEQ ID NO: 264 | QVQLVQSGAEVKKPGASVKVSCKASGYTFTNYWMHWVRQAPGQGLEWIGYVNPRTGYTEYNQKFQGRATLTA | 7B1F3 VH1 |
| | DKSSRTAYMELSSLRSEDTAVYSCARILNGTGFAYWGQGTLVTVSS | |
| SEQ ID NO: 265 | QVQLVQSGAEVKKPGASVKVSCKASGYTFTNYWMHWVRQAPGQGLEWMGYVNPRTGYTEYNQKFQGRVTMTA | 7B1F3 VH2 |
| | DKSTSTAYMELSSLRSEDTAVYYCARILNGTGFAYWGQGTLVTVSS | |
| SEQ ID NO: 266 | QVQLVQSGAEVKKPGASVKVSCKASGYTFTNYWMHWVRQAPGQGLEWIGYVNPRTGYTEYNQQLQGRATLTA | 7B1F3 VH3 |
| | DKSSRTAYMELRSLRSDDTAVYSCARILNGTGFAYWGQGTLVTVSS | |
| SEQ ID NO: 267 | QVQLVQSGAEVKKPGASVKVSCKASGYTFTNYWMHWVRQAPGQGLEWMGYVNPRIGYTEYNQQLQGRVTMTA | 7B1F3 VH4 |
| | DKSTSTAYMELRSLRSDDTAVYYCARILNGTGFAYWGQGTLVTVSS | |
| SEQ ID NO: 268 | DIVLTQSPDSLAVSLGERATINCKSSQSLLYSSNQKNYLAWYQQKPGQPPKLLIYWASTRESGVPDRFSGSG | 7B1F3 VL1 |
| | SGTDFTLTISSLQAEDVAIYYCQQYYSFPLTFGGGTKLEIK | |
| SEQ ID NO: 269 | DIVLTQSPLSLPVIPGEPASISCRSSQSLLYSSNQKNYLAWYLQKPGQSPQLLIYWASTRESGVPDRFSGSG | 7B1F3 VL3 |
| | SGTDEILKISRVEAEDVGIYYCQQYYSFPLTFGGGTKLEIK | |
| SEQ ID NO: 270 | QVQLVQSGAEVKKPGASVKVSCKASGYTFTNYWMHWVRQAPGQGLEWIGYVNPRTGYTEYNQKFQGRATLTA | 7B1F3 VH5 |
| | DKSSRTAYMELSSLRSEDTAVYSCARILQGTGFAYWGQGTLVTVSS | |
| SEQ ID NO: 271 | QVQLVQSGAEVKKPGASVKVSCKASGYTFINYWMHWVRQAPGQGLEWMGYVNPRTGYTEYNQKFQGRVTMTA | 7B1F3 VH6 |
| | DKSTSTAYMELSSLRSEDTAVYYCARILQGTGFAYWGQGTLVTVSS | |
| SEQ ID NO: 272 | MTPTTTTAELTTEFDYDDEATPCVLTDVLNQSKPVTLFLYGVVFLFGSIGNFLVIFTITWRRRIQCSGDVYF | US28 fusion protein, comprising US28 sequence |
| | INLAAADLLFVCTLPLWMQYLLDANSLASVPCTLLTACFYVAMFASLCFITEIALDRYYAIVYMRYRPVKQA | from HCMV VHL/Estrain |
| | CLFSIFWWIFAVIIAIPHFMVVTKKDNQCMTDYDYLEVSYPIILNVELMLGAFVIPLSVISYCYYRISRIVA | |
| | LLYVFVGTKFRQELHCLLAEFRQRLFSRDVSWYHSMSESRRSSPSRRETSSDTLSDEACRVSQIIPAAALEG | |
| | SHHHHHH | |
| SEQ ID NO: 273 | MTPTTITAELTTEFDYDEDATPCVFTDVLNQSKPVTLFLYGVVELEGSIGNFLVIFTITWRRRIQCSGDVYF | US28 fusion protein, comprising US28 sequence |
| | INLAAADLLFVCTLPLWMQYLLDANSLASVPCTLLTACFYVAMFASLCFITEIALDRYYAIVYMRYRPVKQA | from HCMV Merlin strain |
| | CLFSIFWWIFAVIIAIPHFMVVTKKDNQCMTDYDYLEVSYPIILNVELMIGAFVIPLSVISYCYYRISRIVA | |
| | VSQSRHKGRIVRVLIAVVLVEIIFWLPYHLTLFVDTLKLIKWISSSCEFERSLKRALILTESLAFCHCCLNP | |
| | LLYVFVGTKFRQELHCLLAEFRQRLFSRDVSWYHSMSESRRGSPSRRETSSDTLSDEVCRVSQTIPAAALEG | |
| | SHHHHHH | |
| SEQ ID NQs: | (Intentionally Blank) | No sequences corresponding to these Seq ID |
| 274-292 | | numbers |
| SEQ ID NO: 293 | YINPSTGYAEYNQKFQG | VH-CDR2 sequence of 4H3C3, as found in |
| | | 4H3C3 VH1 & 4H3C3 VH2 |
| SEQ ID NO: 294 | YINPSTGYAEYNQKLQG | VH-CDR2 sequence of 4H3C3, as found in |
| | | 4H3C3 VH3 & 4H3C3 VH4 |
| SEQ ID N: 295 | RSSQSLLYSSNQKNYLA | VL-CDR1 sequence of 4H3C3, as found in |
| | | 4H3C3 VL3, 4H3C3 VL4, 7B1F3 VL4 & 7B1F3 |
| | | VL3 |
| SEQ ID NO: 296 | YVNPRTGYTEYNQKFQG | VH-CDR2 sequence of 7B1F3, as found in 7B1F3 |
| | | VH1, 7B1F3 VH2, 781F3 VH5 & 7B1F3 VH6 |
| SEQ ID NO: 297 | YVNPRIGYTEYNQQLQG | VH-CDR2 sequence of 7B1F3, as found in 7B1F3 |
| | | VH3, 7B1F3 VH4 |
| SEQ ID NO: 298 | ILQGTGFAY | VH-CDR3 sequence of 7B1F3, as found in 7B1F3 |
| | | VH5 & 7B1F3 VH6 |
| SEQ ID NO: 299 | Y(I/V)NP(S/R/N)T(G/A)Y(A/T)E(Y/F)NQ(K/Q)(F/L)(K/M/Q)(D/G) | ECD1 V_{H}-CDR2 consensus sequence |
| SEQ ID NO: 300 | (L/I)(R/L)(F/N/S/Q)(G/D)(S/*/R)(S/T)GFAY | ECD1 V_{H}-CDR3 consensus sequence |
| SEQ ID NO: 301 | (K/R)SSQSLLYSSNQKNYLA | ECD1 V_{H}-CDR1 consensus sequence |

TABLE 1
According to the Blast search, 90% of the HCMV strains show ECD
4D variant, The strains showing the 4N variant are listed below:

| | | | Variant US28 | |
|---|---|---|---|---|
| Access Number | Strain Name | Source | ECD3 | Year |
| MT044485.1 | SYD-SCT1 | Plasma | 4N | 2020 |
| KF021605.1 | TR | Eye of HIV-pat | 4N | 2013 |
| AF073835 | N8 | ? | 4N | 2015 |
| KP745645.1 | BE/13/2010 | Urine | 4N | 2015 |
| KP745727.1 | BE/17/2010 | Urine | 4N | 2015 |
| AF073834.1 | L10 | ? | 4N | 2000 |
| KY490076.1 | HANRTR8 | Blood of kidn.trpl.pat. | 4N | 2017 |
| AF073833.1 | FS4 | ? | 4N | 2000 |
| MH836520.1 | P020 | ? | 4N | 2018 |
| MH836506.1 | P003 | ? | 4N | 2018 |
| GU93774 | Toledo | Urine from cong. inf. infant | 4N | 2003 |
| KT959235 | DB | Pregnant woman | 4N | 2009 |

TABLE 2
Comparison of the surface binding specificity
of potential US28 binding antibodies

| | Binding |
|---|---|
| ANTIBODY | Specificity |
| 13-5G6-1D3 clone | 15.2* |
| 13-5G6-1D3 rAb | 21.5* |
| 13-5C6-1B5 clone | 11* |
| 14-1H3-1A6 clone | 26* |
| VUN100 | 4* |
| US28 Commercial Ab | 1.70*** |

The ratio is based on surface binding of the antibody to *CHO-US28-A1 cells compared with the US28 negative control CHO cells; **US28 expressing HEK-293 vs. Mock HEK-293 membranes as reported by De Groof et al. 2019 (supra); ***HCMV Ad169 infected MRC-5 cells vs. Mock MRC-5 cells. Values >1 means higher binding on US28 expressing cells than the negative control cells.

TABLE 3
US28 protein structure encoded by HCMV strain
DB (SEQ ID No: 5/Accession No. KT959235;
mutations are indicated in bold text), and
mutations in the US28 protein encoded by
different HCMV strains:

(SEQ ID No: 5)
MTPTTTTAELTTEFDYDEAATPCVFTDVLNQSKPVTLFLYGVVFL
FGSIGNFLVIFTITWRRRIQCSGDVYFINLAAADLLFVCTLPLWM
QYLLDHNSLASVPCTLLTACFYVAMFASLCFITEIALDRYYAIVY
MRYRPVKQACLFSIFWWIFAVIIAIPHFMVVTKKNNQCMTDYDYL
EVSYPIILNVELMLGAFVIPLSVISYCYYRISRIVAVSQSRHKGR
IVRVLIAVVLVFIIFWLPYHLTLFVDTLKLLKWISSSCEFERSLK
RALILTESLAFCHCCLNPLLYVFVGTKFRQELHCLLAEFRQRLFS
RDVSWYHSMSFSRRSSPSRRETSSDTLSDEVCRVSQIIP

| HCMV DB | |
|---|---|
| (KT959235) vs. | US28 mutations |
| Toledo (GU937742) | Identical (A1 genotype) |
| Towne (FJ616285) | A19D; N170D (A2 genotype) |
| VR1814 (GU179289) | A19D; N170D; S330G |
| | (A2 genotype) |
| TR (KF021605.1) | L45I; R267K (A1 genotype) |
| TB40/E (KF297339) | A8T; D15E; E18L; A19G; |
| | V24T; N170D (B1 genotype) |
| Merlin (AY446894) | A19D; N170D; S330G |
| | (A2 genotype) |
| JP (GQ221975) | N170D (A1 genotype) |
| AD169 (X17403.1) | A19D; N170D (A2 genotype) |
| AF1 (GU179291.1) | N170D (A1 genotype) |
| VHL/E (L20501.1) | E18D; A19E; F25L; N170D; |
| | R267K; V346A (D genotype) |
| Davis (JX512198.1) | A19D, N170D, V190I, R267K, |
| | V346A (A1 genotype) |
| BL (MW980585) | A19D, T21A, F25L, N170D, |
| | V250L, R267K, V346A |
| | (C genotype) |

1-36. (canceled)

37. A binding molecule comprising six complementarity determining region (CDR) sequences corresponding to all six of the CDR sequences of an antibody selected from the group consisting of:
(a) 4H3C3, wherein the CDR1, 2 and 3 sequences of the variable heavy chain (VH) are as defined by SEQ ID NOs: 196, 197 and 198, respectively, and the CDR1, 2 and 3 sequences of the variable light chain (VL) are as defined by SEQ ID NOs: 199, 200 and 201, respectively;
(b) 7B1F3, wherein the CDR1, 2 and 3 sequences of the variable heavy chain (VH) are as defined by SEQ ID NOs: 219, 220 and 221, respectively, and the CDR1, 2 and 3 sequences of the variable light chain (VL) are as defined by SEQ ID NOs: 199, 222 and 223, respectively; and
(c) 2F5B11, wherein the CDR1, 2 and 3 sequences of the variable heavy chain (VH) are as defined by SEQ ID NOs: 242, 243 and 244, respectively, and the CDR1, 2 and 3 sequences of the variable light chain (VL) are as defined by SEQ ID NOs: 199, 245 and 246, respectively.

38. The binding molecule of claim 37, wherein the binding molecule comprises one or more polypeptide chains, and wherein:
(a) said binding molecule has binding specificity to an epitope within a polypeptide consisting of the amino acid sequence of TDVLNQSKPVTL (SEQ ID NO: 177) within the extracellular domain 1 (ECD1) of a US28 protein of human cytomegalovirus (HCMV), wherein ECD1 and SEQ ID NO: 177 of the US28 protein each comprise an amino acid sequence presented in the US28 protein at positions corresponding to positions 1 to 37 and 26 to 37, respectively, of the US28 protein encoded by human cytomegalovirus (HCMV) as set forth in SEQ ID NO: 5;
(b) said binding molecule has binding specificity to an epitope that is present entirely within the amino acid sequence of SEQ ID NO: 177 of the US28 protein of HCMV;
(c) said binding molecule has binding specificity to an epitope that is a linear epitope within the amino acid sequence of SEQ ID NO: 177 of the US28 protein of HCMV; and/or
(d) said binding molecule has a binding specificity to an epitope within the amino acid sequence of SEQ ID NO: 177 of a US28 protein of HCMV that is HCMV strain agnostic,
optionally, wherein the binding molecule:
(i) has a binding specificity to an epitope within a US28 protein of HCMV that is agnostic to two or more (such as all) of HCMV strains, and/or
(ii) has a binding specificity that is agnostic to two or more (such as all) HCMV strains selected from the group consisting of DB, Towne, AF1, VHL/E, AD169, BL, DAVIS, JP, Merlin, PH, TB40/E, Toledo, TR and VR1814 (FIX).

39. The binding molecule of claim 37, wherein:
(a) the binding molecule is selected from the group consisting of: an antibody, a monoclonal antibody, and a chimeric antigen receptor (CAR); and/or
(b) the binding molecule is selected from the group consisting of: a monospecific binding molecule, and a multispecific (for example, bispecific or trispecific) binding molecule, that comprises one or more regions with binding specificity to an epitope within a polypeptide consisting of the amino acid sequence of SEQ ID NO: 177 within extracellular domain 1 (ECD1) of a US28 protein of human cytomegalovirus (HCMV).

40. The binding molecule of claim 37, comprising:
(a) a variable heavy chain (V_{H}) polypeptide that comprises, or consists of, the sequence of SEQ ID NO: 187, and/or a variable light chain (V_{L}) polypeptide that comprises, or consists of, the sequence of SEQ ID NO: 189;
(b) a variable heavy chain (V_{H}) polypeptide that comprises, or consists of, the sequence of SEQ ID NO: 211, and/or a variable light chain (V_{L}) polypeptide that comprises, or consists of, the sequence of SEQ ID NO: 213; or
(c) a variable heavy chain (V_{H}) polypeptide that comprises, or consists of, the sequence of SEQ ID NO: 233, and/or a variable light chain (V_{L}) polypeptide that comprises, or consists of, the sequence of SEQ ID NO: 235.

41. The binding molecule of claim 37, wherein the binding molecule is selected from the group consisting of:
(a) bivalent antibodies, such as IgG-scFv antibodies (for example, wherein a first binding domain is an intact IgG and a second binding domain is an scFv attached to the first binding domain at the N-terminus of a light chain and/or at the C-terminus of a light chain and/or at the N-terminus of a heavy chain and/or at the C-terminus of a heavy chain of the IgG, or vice versa),
(b) monovalent antibodies, such as a DuoBody® or ‘knob-in-hole’ bispecific antibody (for example, an scFv-KIH, scFv-KIHr, a BiTE-KIH or a BiTE-KIHr);
(c) scFv₂-Fc antibodies;
(d) bispecific antibodies, such as bispecific T-cell engager (BiTE) antibodies;
(e) dual variable domain (DVD)-Ig antibodies;
(f) dual-affinity re-targeting (DART)-based antibodies (for example, DART2-Fc or DART);
(g) trispecific antibodies, such as DNL-Fab3 antibodies or trispecific antibodies with binding specificity for an epitope within a polypeptide consisting of the amino acid sequence of TDVLNQSKPVTL (SEQ ID NO: 177) of a US28 protein of HCMV, optionally wherein the trispecific antibody is a trispecific immune cell engager antibody, for example, a trispecific T-cell engager (TiTE), and optionally wherein the TiTE antibody comprises a CD3-binding domain;
(h) scFv-HSA-scFv antibodies;
(i) single domain antibodies;
(j) heavy-chain-only IgGs (hcIgGs), such as camelid IgG (e.g. VHH antibodies) and shark immunoglobulin new antigen receptor (IgNAR), and single chain antibodies thereof;
(k) a chimeric antigen receptor (CAR) comprising an extracellular domain according to claim 37, for example an extracellular domain that comprises any one of options (a) to (h), or combinations thereof;
(l) a bispecific immune cell engager antibody, for example, a bispecific T-cell engager (BiTE),
(n) a BiTE antibody that comprises a CD3-binding domain;
(n) a monoclonal antibody; and
(o) a recombinant monoclonal antibody, for example, a monoclonal antibody produced recombinantly by CHO cells.

42. A functional fragment of the binding molecule of claim 37, wherein the functional fragment comprises or consists of an antigen-binding fragment of a binding molecule as defined by claim 37, or a variant, fusion or derivative thereof selected from the group consisting of: an Fv fragment (such as a single chain Fv fragment (scFv), or a disulphide-bonded Fv fragment), a Fab-like fragment (such as a Fab fragment, a Fab′ fragment or a F(ab)2 fragment), and single domain antibodies (dAbs, including single and dual formats, such as dAb-linker-dAb and nanobodies).

43. The binding molecule of claim 37, wherein the binding molecule comprises a fusion polypeptide sequence, said fusion polypeptide sequence comprising a first amino acid sequence fused to a second amino acid sequence, wherein: the first amino acid sequence comprises or consists of at least one of the polypeptide chains of the binding molecule, and the second amino acid sequence is a fusion partner.

44. A nucleic acid molecule, or combination of multiple distinct nucleic acid molecules, or a vector comprising the nucleic acid molecule, or a vector comprising the combination of multiple distinct nucleic acid molecules, wherein the nucleic acid molecule comprises, or the combination of multiple distinct nucleic acid molecules collectively comprises, one or more nucleic acid sequences that, individually or in combination, encode the binding molecule of claim 37.

45. A cell comprising the nucleic acid molecule, the combination of multiple distinct nucleic acid molecules, or the vector, according to claim 44.

46. A method of producing a cell, the method comprising introducing the nucleic acid molecule, the combination of multiple distinct nucleic acid molecules, and/or the vector, according to claim 44, into a cell.

47. A method of producing a binding molecule, the method comprising: expressing the nucleic acid molecule, the combination of multiple distinct nucleic acid molecules, and/or the vector, according to claim 44, in a cell, and optionally wherein the method further comprises the step of isolating the thus-produced binding molecule from the cell.

48. A conjugate, the conjugate comprising a moiety conjugated to the binding molecule of claim 37, optionally wherein said moiety is a therapeutic, prophylactic, diagnostic, prognostic, or theragnostic moiety, and/or wherein said moiety is a drug (for example, wherein the conjugate is an antibody-drug conjugate (“ADC”)) and/or a radioactive moiety (for example, wherein the conjugate is suitable for use in radioimmunotherapy (“RIT”)).

49. A method of combating HCMV or a disease or condition associated with HCMV, the method comprising administering to a subject, or to ex vivo or in vitro cellular material, the binding molecule of claim 37.

50. A vaccine composition suitable for use in vaccinating against, reducing the risk of, preventing, or combating a disease or condition associated with human cytomegalovirus (HCMV), wherein:
the vaccine is an active or passive vaccine,
the vaccine triggers and/or provides an immune response directed to an epitope present within a polypeptide consisting of the amino acid sequence of TDVLNQSKPVTL (SEQ ID NO: 177) within extracellular domain 1 (ECD1) of a US28 protein of human cytomegalovirus (HCMV), and
said ECD1 and SEQ ID NO: 177 of the US28 protein comprises an amino acid sequence presented in the US28 protein at positions corresponding to positions 1 to 37 and 26 to 37, respectively, of the US28 protein encoded by HCMV as set forth in SEQ ID NO: 5.

51. A method of vaccinating against, reducing the risk of, preventing, and/or combating a disease or condition associated with HCMV, the method comprising administering to a subject the vaccine of claim 50.

52. A method of assessing one or more biological conditions and/or biological characteristics of a subject and/or of ex vivo biological material, wherein the method comprises:
(a) contacting the subject and/or the ex vivo biological material with the binding molecule of claim 37; and
(b) making an assessment of the subject and/or the ex vivo biological material based on a direct and/or indirect measurement of the binding of the binding molecule to the subject and/or the ex vivo biological material.

53. A method of combating a HCMV infection (such as a latent HCMV infection and/or a lytic HCMV infection and/or a multi-strain HCMV infection) in living ex vivo biological material, the method comprising contacting the living ex vivo biological material with the binding molecule of claim 37.

54. Living ex vivo biological material that is obtained, or obtainable, by the method of claim 53.

55. A method of treating a subject in need thereof, comprising administering the ex vivo living biological material of claim 54, to the subject.

56. A method of screening for a binding molecule having binding specificity to a first epitope within a polypeptide consisting of the amino acid sequence of TDVLNQSKPVTL (SEQ ID NO: 177) within extracellular domain 1 (ECD1) of a US28 protein of human cytomegalovirus (HCMV), wherein ECD1 and SEQ ID NO: 177 of the US28 protein comprises an amino acid sequence presented in the US28 protein at positions corresponding to positions 1 to 37 and 26 to 27, respectively, of the US28 protein encoded by HCMV as set forth in SEQ ID NO: 5, the method comprising:
(a) providing one or more peptides or polypeptides comprising, or consisting of, the sequence TDVLNQSKPVTL (SEQ ID NO: 177), or an immunogenic fragment thereof;
(b) providing one or more candidate binding molecules; and
(c) determining the binding specificity and/or binding affinity of one or more candidate binding molecules to the one or more peptides, thereby to select one or more binding molecules having binding specificity to the first epitope within a polypeptide consisting of the amino acid sequence of SEQ ID NO: 177.
